(19) United States
(12) Patent Application Publication
(10) Pub. No.: US 2024/0398990 A1
(43) Pub. Date: Dec. 5, 2024

(54) LIVER-SPECIFIC EXPRESSION CASSETTES, VECTORS AND USES THEREOF FOR EXPRESSING THERAPEUTIC PROTEINS

(71) Applicant: Generation Bio Co., Cambridge, MA (US)

(72) Inventors: Jessica Lynn Keenan, Cambridge, MA (US); Russell Monds, Cambridge, MA (US); Elizabeth Majumdar, Cambridge, MA (US); Jicong Cao, Cambridge, MA (US)

(21) Appl. No.: 18/690,020

(22) PCT Filed: Sep. 16, 2022
(86) PCT No.: PCT/US2022/043884

§ 371 Date: Mar. 7, 2024

Related U.S. Application Data
(60) Provisional application No. 63/245,013, filed on Sep. 16, 2021.

(51) Int. Cl. A61K 48/00; C07K 14/755; C12N 15/85

(52) U.S. Cl. CPC A61K 48/0058; C07K 14/755; C12N 15/85; C12N 2830/008

(57) ABSTRACT

The present disclosure provides liver-specific expression cassettes, vectors comprising the expression cassettes, and uses in gene therapy, particularly liver-directed gene therapy.

### RELATED APPLICATIONS

This application claims priority to U.S. Provisional Application No. 63/245,013, filed on Sep. 16, 2021, the contents of which is hereby incorporated by reference in its entirety.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g., underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome. Such outcomes can be attributed to expression of a therapeutic protein such as an antibody, a functional enzyme, or a fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors.

The liver is directly or indirectly involved in many essential processes and is affected by numerous inherited diseases. Therefore, many inherited diseases could be effectively treated by targeting the liver, using gene transfer approaches. However, there are challenges that remain associated with liver-directed gene therapy, including efficiently targeting hepatocytes, maintaining stability of the vector genome, and achieving persistent high level expression. Among the many virus-derived vectors available (e.g., recombinant retrovirus, recombinant lentivirus, recombinant adenovirus, and the like), recombinant adeno-associated virus (rAAV) has gained popularity as a versatile vector in gene therapy. Liver-directed gene therapy clinical trials with AAV vectors have reported clinical efficacy data (Rodriguez-Marquez et al., Expert Opinion on Biological Therapy Volume 21, 2021—Issue 6). While clinical advances have been made using rAAV vectors for Factor IX (FIX) expression in the liver, the use of rAAV for FVIII expression in hemophilia A patients has been challenging due to ineffective biosynthesis of human FVIII (hFVIII). rAAV vectors produce capsids that have limited space to encapsulate nucleic acids. FVIII is a large glycoprotein, and the rAAV sequences necessary to encode and express FVIII generally exceed the packaging capacity of the capsid.

Recombinant capsid-free AAV vectors can be obtained as an isolated linear nucleic acid molecule comprising an expressible transgene and promoter regions flanked by two wild-type AAV inverted terminal repeat sequences (ITRs) including the Rep binding and terminal resolution sites. These recombinant AAV vectors are devoid of AAV capsid protein encoding sequences, and can be single-stranded, double-stranded or duplex with one or both ends covalently linked through the two wild-type ITR palindrome sequences (e.g., WO2012/123430, U.S. Pat. No. 9,598,703). They avoid many of the problems of AAV-mediated gene therapy in that the transgene capacity is much higher, transgene expression onset is rapid, and the patient immune system recognizes the DNA molecules as a virus to be cleared.

Non-viral gene therapy is assumed to be less toxic for the host and safer in terms of gene delivery compared to a viral vector. One example of a non-viral gene therapy, closed-ended DNA (“ceDNA”) vectors, has many attractive features for gene-based therapy. For example, ceDNA vectors have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., large transgenes, multiple transgenes, regulatory switched, and incorporation of the native genetic regulatory elements of the transgene, if desired.

In most living organisms, and especially in eukaryotes with large genome sizes, however, there does not appear to be a driving force to limit enhancer/promoter size, and therefore most endogenous enhancer/promoters span hundreds, and more often thousands, of base pairs (bp) of DNA. Due to their size, these endogenous natural gene enhancers/promoters are generally not amenable to inclusion in gene therapy products due to size limitations.

Regardless of viral or non-viral delivery, there remains a need for a technology that permits robust expression of a therapeutic protein, such as a liver-specific therapeutic protein, in a cell, tissue or subject, to improve the efficiency and safety of treatment of a genetic disease or disorder.

### SUMMARY

The present disclosure has applied a range of bioinformatic analyses to identify a novel and inventive set of non-natural modifications to a native liver-specific Serpin enhancer region that surprisingly resulted in acute expression level and improved sequence characteristics known to impact expression durability of gene product.

The disclosure also provides an evolutionary conservation analysis to selective removal of CpGs in the enhancer without disrupting function. Enhancers are often combined in series to drive higher levels of transcription initiation. However, the principals underlying optimal number and orientation of enhancer regions remain not well understood. Spacing between transcription factor binding sites is likely a key selection attribute that impacts function, especially considering that DNA is a helix such that number of nucleotides between binding sites also changes their rotational spatial orientation. As described herein, a range of enhancer combinations were tested for improved function, including different numbers of enhancers and nucleotide spacer content. Bioinformatic analysis was used to guide the sequence selection of sequence substitutions tested.

The technology described herein relates to liver-specific nucleic acid expression cassettes comprising specific regulatory elements (enhancer-promoter combination) that have been improved to enhance liver-specific gene expression, such that the native cis-regulatory region has been optimized to minimize CpG content and to enhance spacer optimization, and a vector, either a viral vector (e.g., an AAV-based vector), or a non-viral vector (e.g., a ceDNA vector).

As disclosed herein, the liver-specific expression cassette surprisingly promotes substantially increased protein expression in the liver and in liver cells than in other tissue types, while retaining tissue specificity. In some embodiments, the liver-specific regulatory elements (e.g., enhancer-promoter combination) can be included in a viral vector (such as an adeno-associated virus vector (AAV)) or a non-viral vector a capsid-free (e.g., non-viral) DNA vector with covalently-closed ends (referred to herein as a “closed-ended DNA vector” or a “ceDNA vector”) in operative combination with a heterologous nucleic acid sequence encoding a protein of interest to promote expression of the protein of interest, for example, in liver tissue and/or cells. An advantage of the promoters of the present disclosure is that the enhancer-promoters can be designed and selected for the amount of expression of gene product by the vector, while also ensuring that the amount of promoter is not immunogenic. In some embodiments, the vector (e.g., the AAV vector or ceDNA vector) provides effective expression of the protein of interest at doses that are not predicted to cause immunogenicity in humans. In some embodiments, the vector (e.g., the AAV vector or ceDNA vector) provides effective expression of the protein of interest at doses that are not predicted to cause toxicity in humans. The improvements described herein can be generalized to the improved expression of any transgene (e.g., AAV, ceDNA).

In a first aspect, the disclosure relates to a liver-specific nucleic acid regulatory element comprising a nucleic acid sequence having at least 93% identity to any one of SEQ ID NOs: 1-80, 138 or 139. In one embodiment, the nucleic acid sequence has at least 94% identity to any one of SEQ ID NOs: 1-80, 138 or 139. In one embodiment, the nucleic acid sequence has at least 95% identity to any one of SEQ ID NOs: 1-80, 138 or 139. In one embodiment, the nucleic acid sequence has at least 96% identity to any one of SEQ ID NOs: 1-80, 138 or 139. In one embodiment, the nucleic acid sequence has at least 97% identity to any one of SEQ ID NOs: 1-80, 138 or 139. In one embodiment, the nucleic acid sequence has at least 98% identity to any one of SEQ ID NOs: 1-80, 138 or 139. In one embodiment, the nucleic acid sequence has at least 99% identity to any one of SEQ ID NOs: 1-80, 138 or 139. In one embodiment, the nucleic acid consists of any one of SEQ ID NOs: 1-80, 138 or 139. In one embodiment, the nucleic acid sequence comprises any one of SEQ ID NOs: 1-80, 138 or 139.

In a first aspect, the disclosure relates to a liver-specific nucleic acid regulatory element comprising a nucleic acid sequence having at least 95% identity to SEQ ID NO: 131. In one embodiment, the nucleic acid sequence has at least 96% identity to SEQ ID NO: 131. In one embodiment, the nucleic acid sequence has at least 97% identity to SEQ ID NO: 131. In one embodiment, the nucleic acid sequence has at least 98% identity to SEQ ID NO: 131. In one embodiment, the nucleic acid sequence has at least 99% identity to SEQ ID NO: 131. In one embodiment, the nucleic acid sequence comprises SEQ ID NO: 131. In one embodiment, the nucleic acid sequence consists of SEQ ID NO: 131.

In a first aspect, the disclosure relates to a liver-specific nucleic acid regulatory element comprising a nucleic acid sequence having at least 95% identity to SEQ ID NO: 122. In one embodiment, the nucleic acid sequence has at least 96% identity to SEQ ID NO: 122. In one embodiment, the nucleic acid sequence has at least 97% identity to SEQ ID NO: 122. In one embodiment, the nucleic acid sequence has at least 98% identity to SEQ ID NO: 122. In one embodiment, the nucleic acid sequence has at least 99% identity to SEQ ID NO: 122. In one embodiment, the nucleic acid sequence comprises SEQ ID NO: 122. In one embodiment, the nucleic acid consists of SEQ ID NO: 122.

In a first aspect, the disclosure relates to a liver-specific nucleic acid regulatory element comprising a nucleic acid sequence having at least 95% identity to SEQ ID NO: 81. In one embodiment, the nucleic acid sequence has at least 96% identity to SEQ ID NO: 81. In one embodiment, the nucleic acid sequence has at least 97% identity to SEQ ID NO: 81. In one embodiment, the nucleic acid sequence has at least 98% identity to SEQ ID NO: 81. In one embodiment, the nucleic acid sequence has at least 99% identity to SEQ ID NO: 81. In one embodiment, the nucleic acid sequence comprises SEQ ID NO: 81. In one embodiment, the nucleic acid consists of SEQ ID NO: 81.

In a first aspect, the disclosure relates to a liver-specific nucleic acid regulatory element comprising a nucleic acid sequence having at least 95% identity to SEQ ID NO: 82. In one embodiment, the nucleic acid sequence has at least 96% identity to SEQ ID NO: 82. In one embodiment, the nucleic acid sequence has at least 97% identity to SEQ ID NO: 82. In one embodiment, the nucleic acid sequence has at least 98% identity to SEQ ID NO: 82. In one embodiment, the nucleic acid sequence has at least 99% identity to SEQ ID NO: 82. In one embodiment, the nucleic acid sequence comprises SEQ ID NO: 82. In one embodiment, the nucleic acid consists of SEQ ID NO: 82.

In a first aspect, the disclosure relates to a liver-specific nucleic acid regulatory element comprising a nucleic acid sequence having at least 95% identity to SEQ ID NO: 83. In one embodiment, the nucleic acid sequence has at least 96% identity to SEQ ID NO: 83. In one embodiment, the nucleic acid sequence has at least 97% identity to SEQ ID NO: 83. In one embodiment, the nucleic acid sequence has at least 98% identity to SEQ ID NO: 83. In one embodiment, the nucleic acid sequence has at least 99% identity to SEQ ID NO: 83. In one embodiment, the nucleic acid sequence comprises SEQ ID NO: 83. In one embodiment, the nucleic acid consists of SEQ ID NO: 83.

In another aspect, disclosed herein is a liver-specific nucleic acid regulatory element consisting essentially of a nucleic acid sequence set forth in any one of Table 10, Table 11, Table 12, or Table 13.

In another aspect, disclosed herein is a liver-specific nucleic acid regulatory element comprising a nucleic acid sequence set forth in any one of Table 10, Table 11, Table 12, or Table 13.

In another aspect, disclosed herein is a liver-specific nucleic acid regulatory element comprising a nucleic acid sequence having at least 93%, at least 94%, at least 95%, at least 96%, at least 97%, at least 98%, at least 99% identity to a sequence set forth in any one of Table 10, Table 11, Table 12, or Table 13.

In one embodiment, the element comprises at least two nucleic acid sequences set forth in any one of Table 10, Table 11, Table 12, or Table 13. In one embodiment, the two nucleic acid sequences are identical. In one embodiment, the element comprises three (3) nucleic acid sequences set forth in any one of Table 10, Table 11, Table 12, or Table 13, optionally wherein the three sequences are identical. In one embodiment, the element consists essentially of two (2) to ten (10) nucleic acid sequences set forth in any one of Table 10, Table 11, Table 12, or Table 13.

In one embodiment, the element comprises a spacer placed between the nucleic acid sequences set forth in any one of Table 10, Table 11, Table 12, or Table 13. In one embodiment, the spacer is at least 2, 3, 4, 5, 6, 7, 8, 9, 10, 11, 12, 13, 14, or 15 base pairs long.

In one embodiment, the element comprises a nucleic acid sequence at least 95%, 96%, 97%, 98% or 99% identical to:

(SEQ ID NO: 223)
GGGGGAGGCTGCTGGTGAATATTAACCAAGGTCACCCCAGTTATCGGAGG
AGCAAACAGGGGCAAAGTCCAC,
(SEQ ID NO: 1381)
GGGGGAAGCTACTGGTGAATATTAACCAAGGTCACCCAGTTATCAGGGAG
CAAACAGGAGCAAAGTCCAT,
(SEQ ID NO: 1073)
GGAGGCTGTTGGTGAATATTAACCAAGGTCACCTCCGTTATCGGAGGAGC
AAACAAGGGCTAAGTCCAC,
or
(SEQ ID NO: 1113)
GGAGGCTGTTGGTGAATATTAACCAAGGTCACCTCAGTTATCGGAGGAGC
AAACAAGGGCAAAGTCCAC.

In one embodiment, the element comprises a nucleic acid consisting of:

(SEQ ID NO: 223)
GGGGGAGGCTGCTGGTGAATATTAACCAAGGTCACCCCAGTTATCGGAGG
AGCAAACAGGGGCAAAGTCCAC,
(SEQ ID NO: 1381)
GGGGGAAGCTACTGGTGAATATTAACCAAGGTCACCCAGTTATCAGGGAG
CAAACAGGAGCAAAGTCCAT,
(SEQ ID NO: 1073)
GGAGGCTGTTGGTGAATATTAACCAAGGTCACCTCCGTTATCGGAGGAGC
AAACAAGGGCTAAGTCCAC,
or
(SEQ ID NO: 1113)
GGAGGCTGTTGGTGAATATTAACCAAGGTCACCTCAGTTATCGGAGGAGC
AAACAAGGGCAAAGTCCAC

In another aspect, the disclosure relates to a liver-specific nucleic acid regulatory element comprising a nucleic acid sequence having at least 85% identity to any one of SEQ ID NOs: 81, 82, 122, 83 or 85. In one embodiment, the nucleic acid sequence has at least 90% identity to any one of SEQ ID NOs: 81, 82, 122, 83 or 85. In one embodiment, the nucleic acid sequence has at least 91% identity to any one of SEQ ID NOs: 81, 82, 122, 83 or 85. In one embodiment, the nucleic acid sequence has at least 92% identity to any one of SEQ ID NOs: 81, 82, 122, 83 or 85. In one embodiment, the nucleic acid sequence has at least 93% identity to any one of SEQ ID NOs: 81, 82, 122, 83 or 85. In one embodiment, the nucleic acid sequence has at least 94% identity to any one of SEQ ID NOs: 81, 82, 122, 83 or 85. In one embodiment, the nucleic acid sequence has at least 95% identity to any one of SEQ ID NOs: 81, 82, 122, 83 or 85. In one embodiment, the nucleic acid sequence has at least 96% identity to any one of SEQ ID NOs: 81, 82, 122, 83 or 85. In one embodiment, the nucleic acid sequence has at least 97% identity to any one of SEQ ID NOs: 81, 82, 122, 83 or 85. In one embodiment, the nucleic acid sequence has at least 98% identity to any one of SEQ ID NOs: 81, 82, 122, 83 or 85. In one embodiment, the nucleic acid sequence has at least 99% identity to any one of SEQ ID NOs: 81, 82, 122, 83 or 85. In one embodiment, the nucleic acid sequence comprises any one of SEQ ID NOs: 81, 82, 122, 83 or 85. In one embodiment, the nucleic acid sequence consists of any one of SEQ ID NOs: 81, 82, 122, 83 or 85.

In another aspect, the disclosure provides a liver-specific expression cassette comprising at least one liver-specific regulatory element of any one of the aspects and embodiments herein. In one embodiment, the liver-specific expression cassette further comprises a liver-specific promoter operably linked to a transgene. In one embodiment, two or more nucleotides separate each liver-specific nucleic acid regulatory element. In one embodiment, 5 or more nucleotides separate each liver-specific nucleic acid regulatory element. In one embodiment, 10 or more nucleotides separate each liver-specific nucleic acid regulatory element. In one embodiment, 15 or more nucleotides separate each liver-specific nucleic acid regulatory element. In one embodiment, 20 or more nucleotides separate each liver-specific nucleic acid regulatory element. In one embodiment, 25 or more nucleotides separate each liver-specific nucleic acid regulatory element. In one embodiment, between 2 and 30 nucleotides separate each liver-specific regulatory element, e.g., 2, 3, 4, 5, 6, 7, 8, 9, 10, 11, 12, 13, 14, 15, 15, 17, 18, 19, 20, 21, 22, 23, 24, 25, 26, 27, 28, 29 or 30 nucleotides.

In another aspect, the disclosure provides a liver-specific expression cassette comprising at least three repeats of a liver-specific nucleic acid regulatory element and a liver-specific promoter operably linked to a transgene, wherein the liver-specific nucleic acid regulatory element comprises a nucleic acid sequence having at least 95% identity to any one of SEQ ID NOs: 81-137, and wherein two or more nucleotides separate each liver-specific nucleic acid regulatory element.

In one embodiment, between 2 and 30 nucleotides separate each regulatory element. In one embodiment, between 2 and 10, between 5 and 15, between 10 and 15, between 10 and 20, between 15 and 25, between 20 and 30 or between 25 and 30 nucleotides separate each regulatory element. In one embodiment, 5 nucleotides separate each regulatory element. In one embodiment, 11 nucleotides separate each regulatory element. In one embodiment, 30 nucleotides separate each regulatory element. In one embodiment, the liver-specific expression cassette comprises two, three, four, or five repeats of the liver-specific nucleic acid regulatory element. In one embodiment, the liver-specific expression cassette comprises six, seven, eight, nine or ten repeats of the liver-specific nucleic acid regulatory element. In one embodiment, liver-specific expression cassette comprises one or more FOXA and HNF4 transcription factor consensus sites. In one embodiment, the liver-specific nucleic acid regulatory element comprises one or more sites of CpG minimization. In one embodiment, the liver-specific promoter is selected from the group consisting of: a transthyretin (TTR) promoter, minimal TTR promotor (TTRm), an AAT promoter, an albumin (ALB) promotor or minimal promoter, an apolipoprotein A1 (APOA1) promoter or minimal promoter, a complement factor B (CFB) promoter, a ketohexokinase (KHK) promoter, a hemopexin (HPX) promoter or minimal promoter, a nicotinamide N-methyltransferase (NNMT) promoter or minimal promoter, a carboxylesterase 1 (CES1) promoter or minimal promoter, a protein C (PROC) promoter or minimal promoter, an apolipoprotein C3 (APOC3) promoter or minimal promoter, a mannan-binding lectin serine protease 2 (MASP2) promoter or minimal promoter, a hepcidin antimicrobial peptide (HAMP) promoter or minimal promoter, and a serpin peptidase inhibitor, clade C (antithrombin), member 1 (SERPINC1) promoter or minimal promoter. In one embodiment, the promoter comprises any sequence from Table 1. In one embodiment, the liver-specific promoter is a TTR promoter or a TTRm promoter. In one embodiment, the transgene encodes a liver-specific therapeutic protein. In one embodiment, the liver-specific therapeutic protein is coagulation factor VIII (FVIII). In one embodiment, the coagulation FVIII comprises a codon optimized nucleic acid sequence. In one embodiment, the coagulation FVIII comprises a nucleic acid sequence having at least 90%, 91%, 92%, 93%, 94%, 95%, 96%, 97%, 98%, 99% identity to, comprises, or consists of SEQ ID NO: 143.

In another aspect, the disclosure provides a vector comprising the liver-specific nucleic acid regulatory element of any one of the aspects or embodiments herein or the liver-specific expression cassette according to any one of the aspects or embodiments herein. In one embodiment, the vector is a viral vector or a non-viral vector. In one embodiment, the vector is a plasmid. In one embodiment, the vector is a closed-ended DNA (ceDNA) vector.

In another aspect, the disclosure provides a pharmaceutical composition comprising the liver-specific expression cassette according to any one of the aspects or embodiments herein or the vector according to any one of the aspects or embodiments herein, and a pharmaceutically acceptable excipient.

In another aspect, the disclosure provides a method of treating a liver-specific disease or disorder comprising transduction or transfection of the vector according to any one of the aspects and embodiments herein, or the pharmaceutical composition of the aspects or embodiments herein, into a subject. In one embodiment, the subject is a human subject suffering from a genetic disorder. In one embodiment, the subject has hemophilia A. In one embodiment, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, progressive familial intrahepatic cholestasis (PFIC) type I (ATP8B1 deficiency), type II (ABCB11), type III (ABCB4), or type IV (TJP2) and Cathepsin A deficiency.

In another aspect, disclosed herein is amethod of increasing expression capacity of a liver-specific enhancer element comprising the nucleic acid sequence CTAAG, comprising introducing a single nucleotide substitution (T to A) mutation such that the substitution results in the nucleic acid sequence comprising CAAAG.

In another aspect, disclosed herein is a liver-specific enhancer element comprising a nucleic acid sequence selected from: CAAAG; CAAAGT; CAAAGTC; GCAAAGT; GCAAAG; or GCAAAGTC.

These and other aspects of the disclosure are described in further detail below.

### DESCRIPTION OF DRAWINGS

FIG. 1A and FIG. 1B depict sequences and alignment of conserved enhancer regions of human and 20 other vertebrates. 115 non-human vertebrate genomes were assessed for conserved SERPINA1 enhancer regions using the UCSC multiz100way and multiz30way multiple alignments. Highlighted nucleotides in the aligned sequences represent differences from the human reference sequence.

FIG. 2 depicts identification of near-consensus binding sites for various transcription factors (TF) in human SERPINA1 enhancer (hSerpEnh) region, including HNF4 and FOXA, which are key regulators of hepatic gene expression. The arrows in the bottom three rows represent TF binding motifs described by Chuah et al. (2014). The arrows at the top 15 rows represent transcription factor (TF) binding motifs identified by independent analyses described herein. Positions where the human SERPINA1 sequence differs from the most highly preferred nucleotide in the sequence logos are boxed.

FIG. 3 depicts multiple bioinformatic analyses employed to inform potential removal of CpG (i.e., CpG ablation). The human SERPINA1 enhancer contains one internal CpG and the potential to form CpGs at its 5′ and 3′ ends (highlighted in red and boxed in the “hSerpEnh” track). Low sequence conservation, the presence of human SNPs that are not known to be associated with disease, and the absence of predicted TF binding sites were assessed to inform sequence changes to ablate the central CpG and the remove potential for CpG formation at the ends of the sequence.

FIG. 4A depicts results of the top 11 constructs (plasmid) in a screen of 30 single (1×) variants using luciferase reporter assay (n=3) in vitro. Results are grouped by rationally designed enhancer variants (1× TFBS Consensus Variants) or conserved SERPINA1 enhancer regions identified in other species (1× Conserved Genomic Variants). Error bars represent standard deviation.

FIG. 4B depicts the sequence design of the top variant in this screen, hSerpEnh_FOXA_HNF4_consensus_v1. hSerpEnh_FOXA_HNF4_consensus_v1 was designed by modifying the FOXA and HNF4 motifs identified in the human SERPINA1 enhancer to match their respective consensus sequences (GTGAATA to GTAAACA for FOXA and CTAAGT to CAAACT for HNF4). The internal CpG was ablated by changing the G, which both has lower sequence conservation than the C and is at the position of a human SNP, to an A to match the SNP.

FIG. 5 depicts results of a screen of 10 multimerzied variants in plasmid using an in vitro (HepG2 cells) luciferase reporter assay (n=3). Results are grouped by 3× repeats of rationally designed enhancer variants (3× TFBS Variants), 3× repeats of conserved SERPINA1 enhancer regions identified in other species (3× Conserved Variant), 3× repeats of the human SERPINA1 enhancer separated by spacers of varying lengths and sequences (3× hSerpEnh Spacer Variants), and enhancers with varying numbers of repeats (#Repeat Variants). The wild-type human SERPINA1 enhancer are labelled (wt). The comparison between the 3× human SERPINA1 enhancer variant and the 3× top performing variant is boxed. Two sets of technical triplicates were performed for the 1× and 3× human enhancers and the top performing 3× variant (r1, r2). Error bars represent standard deviation.

FIG. 6A is a schematic for optimization of spacer sequence to improve performance of hSerpEnh variant repeats. The length and sequence of spacers between SERPINA1 enhancer variant repeats were modified to screen for sequences that improved enhancer function. Spacers of length 2, 3, 5, 11, and 30 were designed to prevent introduction of CpGs or ATGs that may create cryptic translation start sites. 11 nt and 30 nt spacers that contain consensus FOXA and HNF4 binding sites were also designed and tested.

FIG. 6B depicts three main configurations of enhancer elements for screening of improved enhancer variants. The enhancer variants were tested in two main configurations: (1) as a single copy of the enhancer variant upstream of the transthyretin (TTR) promoter, TTR 5′ UTR, and the minute virus of mice (MVM) intron or (2) as three copies of the enhancer variant upstream of the TTR enhancer, TTR promoter, TTR 5′ UTR, and the MVM intron.

FIGS. 7A-7D depict expression levels of FVIII constructs having multimeric repeats of Serpin enhancer variants compared to multimeric human Serpin enhancer (3×, 5× and 10×) variants. FIG. 7A depicts expression levels of 3× HNF_FOXA_v1 variants having CpG minimization, GC rich regions (I-motif secondary structures) minimization, or Aspacer (no spacer) performed equivalent to the level seen in 3× hSerpEnh. However, HNF4 FOXA v1 variants repeated 10 times (10×) did not exhibit a meaningful level of FVIII (see, e.g., FIG. 7C), suggesting that the Serpin enhancer exhibits better performance when it is repeated in a certain number, e.g., 3× to 5×, with 3× preference, but not when it is repeated in an excessive number (e.g., 10×). A consistent observation was made with other Serpin Enhancer elements including, for example, that of bushbaby Serpin enhancer, Chinese tree shrew Serpin enhancer, and human Serpin enhancer (hSerpEnh). In particular, 5× and 10× bushbaby Serpin enhancer element did not exhibit detectable expression levels of FVIII when a plasmid containing the element operably linked to FVIII was injected hydrodynamically into a mouse (FIG. 7D).

FIGS. 8A-FIG. 8E depict FVIII expression as measured by FVIII activity obtained from the serum of mice hydrodynamically injected with a plasmid containing various spacer variants (two-nucleotide long spacers (2-mer; FIG. 8A), three-nucleotide long spacers (3-mer; FIG. 8B), five-nucleotide long spacers (5-mer; FIG. 8C), eleven-nucleotide long spacers (11-mer; FIG. 8D), and thirty nucleotide long spacers (30-mer; FIG. 8E).

FIG. 9 depicts a chart showing the result of FVIII expression using various spacer variants of hSerpEnh (2mers and 11 mers as spacers) and other Serpin enhancer variants (3× bushbaby Serpin enahancer to 3× Chinese tree shrew Serpin Enhancer). One dose of 50 ng plasmid was hydrodynamically injected to Rag2 mice on day 0 with a single terminal collection at day 3 (˜72 hr post dose).

FIG. 10 depicts a chart showing the result of FVIII expression using various spacer variants of hSerpEnh (2mers and 11 mers) and other Serpin enhancer variants (3× bushbaby Serpin enahancer to 3× Chinese tree shrew Serpin Enhancer). One dose of ceDNA was hydrodynamically injected to Rag2 mice on day 0 with a single terminal collection at day 3 (˜72 hr post dose).

FIG. 11 depicts an exemplary annotated nucleotide sequence of a plasmid containing a FVIII ceDNA construct comprising 3× Bushbaby_Aspacers Serpin enhancer element linked to TTRe, TTR liver-specific promoter, MVM intron, codon optimized B-domain deleted FVIII (hFVIII-F309S-BD226seq124-BDD-F309), WPRE 3′UTR, and bGH (SEQ ID NO: 146).

FIG. 12 depicts an annotated nucleotide sequence of a plasmid containing a FVIII ceDNA construct comprising 3× human Serpin enhancer element linked to TTRe_PromoterSet, Consensus_Kozak, codon optimized hFVIII (hFVIII-F309S-BD226seq124-BDD-F309), PacI_site, WPRE_3pUTR, and bGH (SEQ ID NO: 147).

FIG. 13 depicts FVIII expression levels in mice dosed hydrodynamically via tail venin injection with ceDNA constructs having various FVIII and Serpin Enhancer combinations, at Day 0 at a low dose of 0.5 mg/kg or a high dose of 2.0 mg/kg (n=5). Factor VIII expression was measured at Days 7, 14, 21, and 28. Expression of FVIII derived from 3× human SerpEnh having 2 bp spacer and 11 bp spacer were compared with 3× human SerpEnh without a spacer

FIG. 14 depicts FVIII expression levels in mice dosed hydrodynamically via tail vein injection with ceDNA constructs having various FVIII and Serpin Enhancer combinations at Day 0 at a dose of 50 ng (n=5). Factor VIII expression was measured at Days 1 and 3.

FIG. 15 depicts FVIII expression levels in mice dosed hydrodynamically via tail vein injection at Day 0 at a dose of 10 ng (n=5). Factor VIII expression was measured at Day 3.

FIG. 16A and FIG. 16B depict FVIII expression levels in mice treated via hydrodynamic tail vein injection with ceDNA constructs having various FVIII and Serpin Enhancer combination (3× Tibetan antelope SERPINA1; 3× Armadillo CpG minimized SERPINA1; 3× Chinese Tree Shrew and 3× Chinese Tree Shrew CpG minimized; and 3× Bushbaby Aspacer) at Day 0 at three different dose levels: 25 ng/an, 50 ng/an, 100 ng/an (n=4). Factor VIII expression was measured at Day 3.

FIG. 17 depicts an annotated map of pHTS002L, a plasmid employed in making a library of enhancer-luciferase constructs.

FIGS. 18A-18D depict comparisons for two biological replicates of barcode counts for each RNA sample normalized to the corresponding barcode counts for an input DNA sample which were mapped back to their associated enhancer sequences (custom MATLAB script).

FIG. 19 depicts alignment of multiple SERPINA1 enhancer sequences.

### DETAILED DESCRIPTION

Provided herein are liver-specific promoters, wherein the native cis-regulatory region has been optimized to minimize CpG content and to enhance spacer optimization. The liver-specific promoters of the present disclosure represent an improvement over those previously known by providing enhanced efficiency and safety for liver-specific gene therapy.

### Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms, “administration,” “administering” and variants thereof refers to introducing a composition or agent (e.g., a therapeutic nucleic acid or an immunosuppressant as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. The introduction of a composition or agent into a subject is by electroporation. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), guide RNA (gRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, an “effective amount” or “therapeutically effective amount” of a therapeutic agent, such as a FVIII therapeutic protein or fragment thereof, is an amount sufficient to produce the desired effect, e.g., treatment or prevention of hemophilia A. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. In one embodiment, the disease, disorder or condition is hemophilia A. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10^{th} Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “heterologous nucleic acid sequence” and “transgene” are used interchangeably and refer to a nucleic acid of interest (other than a nucleic acid encoding a capsid polypeptide) that is incorporated into and may be delivered and expressed by a ceDNA vector as disclosed herein. In one embodiment, a nucleic acid sequence may be a heterologous nucleic acid sequence. In one embodiment, the term “heterologous nucleic acid” is meant to refer to a nucleic acid (or transgene) that is not present in, expressed by, or derived from the cell or subject to which it is contacted.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), guide RNA (gRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to an uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular FVIII protein is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or ncRNA”).

As used herein, the term “fusion protein” as used herein refers to a polypeptide which comprises protein domains from at least two different proteins. For example, a fusion protein may comprise (i) a therapeutic protein, or a fragment thereof (e.g., FVIII or a fragment thereof) and (ii) at least one non-GOI protein. Fusion proteins encompassed herein include, but are not limited to, an antibody, or Fc or antigen-binding fragment of an antibody fused to a therapeutic protein (e.g., a FVIII protein), e.g., an extracellular domain of a receptor, ligand, enzyme or peptide. The protein or fragment thereof that is part of a fusion protein can be a monospecific antibody or a bispecific or multispecific antibody.

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the disclosure herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleic acid sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C‘ and B-B’ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the modified ITR can be considered substantially symmetrical, even if it has some nucleic acid sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleic acid sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleic acid sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. In one embodiment, the condition is hemophilia A. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “minimize”, “reduce”, “decrease,” and/or “inhibit” (and like terms) generally refers to the act of reducing, either directly or indirectly, a concentration, level, function, activity, or behavior relative to the natural, expected, or average, or relative to a control condition.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis—acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 140), an RBS sequence identified in AAV2. Any known RBS sequence may be used in the embodiments of the disclosure, including other known AAV RBS sequences and other naturally known or synthetic RBS sequences. Without being bound by theory it is thought that he nuclease domain of a Rep protein binds to the duplex nucleic acid sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 140). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the disclosure, including other known AAV TRS sequences and other naturally known or synthetic TRS sequences such as AGTT (SEQ ID NO: 1690), GGTTGG, AGTTGG, AGTTGA, and other motifs such as RRTTRR.

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “neDNA” or “nicked ceDNA” refers to a closed-ended DNA having a nick or a gap of 1-100 base pairs in a stem region or spacer region 5′ upstream of an open reading frame (e.g., a promoter and transgene to be expressed).

As used herein, the terms “gap” refers to a discontinued portion of synthetic DNA vector of the present disclosure, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the terms “sense” and “antisense” refer to the orientation of the structural element on the polynucleotide. The sense and antisense versions of an element are the reverse complement of each other.

As used herein, the term “synthetic AAV vector” and “synthetic production of AAV vector” refers to an AAV vector and synthetic production methods thereof in an entirely cell-free environment.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest, such as FVIII. Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a target gene, e.g., heterologous target gene, encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

In one embodiment, the promoter contained in the nucleic acid expression cassettes and vectors disclosed herein is a liver-specific promoter.

The term “liver-specific promoter” encompasses any promoter that confers liver-specific expression to a (trans)gene. Non-limiting examples of liver-specific promoters are provided on the Liver-specific Gene Promoter Database (LSPD, rulai.cshl.edu/LSPD/), and include, for example, the transthyretin (TTR) promoter or TTR-minimal promoter (TTRm), the alpha 1-antitrypsin (AAT) promoter, the albumin (ALB) promotor or minimal promoter, the apolipoprotein A1 (APOA1) promoter or minimal promoter, the complement factor B (CFB) promoter, the ketohexokinase (KHK) promoter, the hemopexin (HPX) promoter or minimal promoter, the nicotinamide N-methyltransferase (NNMT) promoter or minimal promoter, the (liver) carboxylesterase 1 (CES1) promoter or minimal promoter, the protein C (PROC) promoter or minimal promoter, the apolipoprotein C3 (APOC3) promoter or minimal promoter, the mannan-binding lectin serine protease 2 (MASP2) promoter or minimal promoter, the hepcidin antimicrobial peptide (HAMP) promoter or minimal promoter, and the serpin peptidase inhibitor, clade C (antithrombin), member 1 (SERPINC1) promoter or minimal promoter.

In some embodiments, the promoter is a mammalian liver-specific promoter, in particular a murine or human liver-specific promoter.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

Regulatory elements comprise at least one transcription factor binding site (TFBS), more in particular at least one binding site for a tissue-specific transcription factor, most particularly at least one binding site for a liver-specific transcription factor. Typically, regulatory elements as used herein increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g., in the promoter region) or downstream (e.g., in the 3′UTR) of the gene they regulate in vivo, and may be located in the immediate vicinity of the gene or further away. Although regulatory elements as disclosed herein typically are naturally occurring sequences, combinations of (parts of) such regulatory elements or several copies of a regulatory element, i.e., non-naturally occurring sequences, are themselves also envisaged as regulatory element. Regulatory elements as used herein may be part of a larger sequence involved in transcriptional control, e.g., part of a promoter sequence. However, regulatory elements alone are typically not sufficient to initiate transcription, but require a promoter to this end.

In one embodiment, the one or more regulatory elements contained in the nucleic acid expression cassettes and vectors disclosed herein are preferably liver-specific. Non-limiting examples of liver-specific regulatory elements are disclosed in WO 2009/130208, incorporated by reference in its entirety herein. Another example of a liver-specific regulatory element is a regulatory element derived from the transthyretin (TTR) gene, also referred to herein as “TTRe.” “Liver-specific expression”, as used herein, refers to the preferential or predominant expression of a (trans)gene (as RNA and/or polypeptide) in the liver as compared to other tissues. In one embodiment, at least 50% of the (trans)gene expression occurs within the liver. According to some embodiments, at least 60%, at least 70%, at least 75%, at least 80%, at least 85%, at least 90%, at least 95%, at least 97%, at least 99% or 100% of the (trans)gene expression occurs within the liver. In one embodiment, liver-specific expression entails that there is no ‘leakage’ of expressed gene product to other organs, such as spleen, muscle, heart and/or lung. It is to be understood that, where liver-specific is mentioned in the context of expression, hepatocyte-specific expression is also explicitly envisaged. Similarly, where tissue-specific expression is used in the application, cell-type specific expression of the cell type(s) predominantly making up the tissue is also envisaged.

As used herein, the term “liver cells” encompasses the cells predominantly populating the liver and encompasses mainly hepatocytes, oval cells, liver sinusoidal endothelial cells (LSEC) and cholangiocytes (epithelial cells forming the bile ducts).

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a DNA sequence, e.g., heterologous DNA sequence, that is operably linked to a promoter or other regulatory sequence sufficient to direct transcription of the transgene in the ceDNA vector. Suitable promoters include, for example, tissue specific promoters or promoters of AAV origin.

The term “subject” as used herein refers to a human or animal, to whom treatment, including prophylactic treatment, with the ceDNA vector according to the present disclosure, is provided. Usually, the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

The term “control” as used herein is meant to refer to a reference standard. In one embodiment, a control may be a negative control sample obtained from a healthy patient. According to other embodiments, the control is a positive control sample obtained from a patient diagnosed with a genetic disease or disorder (e.g., hemophilia). In one embodiment, the control is a historical control or a standard reference value or a range of values (such as a previously tested control sample, such as a group of hemophilia A patients with a known prognosis or outcome, or a group of samples representing baseline or normal values).

A difference between a test sample and a control can be an increase or, conversely, a decrease. The difference can be a qualitative difference or a quantitative difference, for example, a statistically significant difference. In some examples, a difference is an increase or decrease, relative to a control, of at least about 5%, such as at least about 10%, at least about 20%, at least about 30%, by less than about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500% or more than 500%.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found, and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleic acid sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. A vector can include nucleic acid sequences that allow it to replicate in a host cell, such as an origin of replication. A vector can also include one or more selectable marker genes and other genetic elements. The term “vector” encompasses any genetic element that is capable of replication when associated with the proper control elements and that can transfer gene sequences to cells. In some embodiments, a vector can be an expression vector or recombinant vector. In some embodiments, the vector is an expression vector that contains the regulatory sequences necessary to allow transcription and translation of the inserted gene (s). In some embodiments, the vector is a ceDNA vector. In some embodiments, the vector is an AAV vector. In some embodiments, the vector is a retroviral gamma vector, a lentiviral vector, or an adenoviral vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a nucleic acid sequence, e.g., heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassemia, xeroderma pigmentosum, Fanconi's anemia, retinitis pigmentosa, ataxia telangiectasia, Bloom's syndrome, retinoblastoma, and Tay-Sachs disease.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.”, is derived from the Latin exempli gratia and is used herein to indicate a non-limiting example. Thus, the abbreviation “e.g.” is synonymous with the term “for example.”

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### II. Expression Cassettes Optimized for Liver-Specific Expression

The present disclosure provides liver-specific expression cassettes to enhance transcription in liver tissue and/or cells. As discussed in the Examples, the present disclosure provides a novel set of non-natural modifications to a native liver-specific enhancer region that unexpectedly increase acute protein expression level and improve sequence characteristics known to impact protein expression durability.

In one embodiment, the liver-specific expression cassette provided herein comprises an enhancer nucleic acid sequence. In one embodiment, the liver-specific expression cassette provided herein comprises more than one repeated enhancer nucleic acid sequences. In one embodiment, the liver-specific expression cassette provided herein comprises two repeated enhancer nucleic acid sequences. In one embodiment, the liver-specific expression cassette provided herein comprises three repeated enhancer nucleic acid sequences. In one embodiment, the liver-specific expression cassette provided herein comprises five repeated enhancer nucleic acid sequences. In one embodiment, the liver-specific expression cassette provided herein comprises between two and 10 repeated enhancer nucleic acid sequences. In one embodiment, the liver-specific expression cassette provided herein comprises ten repeated enhancer nucleic acid sequences. In one embodiment, the liver-specific expression cassette provided herein comprises between 3 and 10 repeated enhancer nucleic acid sequences. In one embodiment, the liver-specific expression cassette comprises more than three repeated enhancer nucleic acid sequences.

In one embodiment, the liver-specific expression cassette comprises two or more repeated enhancer nucleic acid sequences, wherein at least two nucleic acids (2 mer) separate each repeated enhancer nucleic acid sequence. In one embodiment, the liver-specific expression cassette comprises three or more repeated enhancer nucleic acid sequences, wherein at least two nucleic acids separate each repeated enhancer nucleic acid sequence. In one embodiment, the liver-specific expression cassette comprises five or more repeated enhancer nucleic acid sequences, wherein at least two nucleic acids separate each repeated enhancer nucleic acid sequence. In one embodiment, the liver-specific expression cassette comprises ten or more repeated enhancer nucleic acid sequences, wherein at least two nucleic acids separate each repeated enhancer nucleic acid sequence. In one embodiment, the liver-specific expression cassette comprises about 3 to 10 repeated enhancer nucleic acid sequences, wherein at least two nucleic acids separate each repeated enhancer nucleic acid sequence.

In one embodiment, the liver-specific expression cassette provided herein comprises two or more repeated enhancer nucleic acid sequences, wherein at least three nucleic acids (3 mer) separate each repeated enhancer nucleic acid sequence. In one embodiment, the liver-specific expression cassette comprises three or more repeated enhancer nucleic acid sequences, wherein at least three nucleic acids separate each repeated enhancer nucleic acid sequence. In one embodiment, the liver-specific expression cassette comprises five or more repeated enhancer nucleic acid sequences, wherein at least three nucleic acids separate each repeated enhancer nucleic acid sequence. In one embodiment, the liver-specific expression cassette comprises ten or more repeated enhancer nucleic acid sequences, wherein at least three nucleic acids separate each repeated enhancer nucleic acid sequence. In one embodiment, the liver-specific expression cassette comprises between 3 and 10 repeated enhancer nucleic acid sequences, wherein at least three nucleic acids separate each repeated enhancer nucleic acid sequence.

In one embodiment, the liver-specific expression cassette provided herein comprises two or more repeated enhancer nucleic acid sequences, wherein at least five nucleic acids (5 mer) separate each repeated enhancer nucleic acid sequence. In one embodiment, the liver-specific expression cassette comprises three or more repeated enhancer nucleic acid sequences, wherein at least five nucleic acids separate each repeated enhancer nucleic acid sequence. In one embodiment, the liver-specific expression cassette comprises five or more repeated enhancer nucleic acid sequences, wherein at least five nucleic acids separate each repeated enhancer nucleic acid sequence. In one embodiment, the liver-specific expression cassette comprises ten or more repeated enhancer nucleic acid sequences, wherein at least five nucleic acids separate each repeated enhancer nucleic acid sequence. In one embodiment, the liver-specific expression cassette comprises about 3 to 10 repeated enhancer nucleic acid sequences, wherein at least five nucleic acids separate each repeated enhancer nucleic acid sequence.

In one embodiment, the liver-specific expression cassette provided herein comprises two or more repeated enhancer nucleic acid sequences, wherein at least 11 nucleic acids (11 mer) separate each repeated enhancer nucleic acid sequence. In one embodiment, the liver-specific expression cassette comprises three or more repeated enhancer nucleic acid sequences, wherein at least 11 nucleic acids separate each repeated enhancer nucleic acid sequence. In one embodiment, the liver-specific expression cassette comprises five or more repeated enhancer nucleic acid sequences, wherein at least 11 nucleic acids separate each repeated enhancer nucleic acid sequence. In one embodiment, the liver-specific expression cassette comprises ten or more repeated enhancer nucleic acid sequences, wherein at least 11 nucleic acids separate each repeated enhancer nucleic acid sequence. In one embodiment, the liver-specific expression cassette comprises about 3 to 10 repeated enhancer nucleic acid sequences, wherein at least 11 nucleic acids separate each repeated enhancer nucleic acid sequence.

In one embodiment, the liver-specific expression cassette provided herein comprises two or more repeated enhancer nucleic acid sequences, wherein at least 30 nucleic acids (30 mer) separate each repeated enhancer nucleic acid sequence. In one embodiment, the liver-specific expression cassette comprises three or more repeated enhancer nucleic acid sequences, wherein at least 30 nucleic acids separate each repeated enhancer nucleic acid sequence. In one embodiment, the liver-specific expression cassette comprises five or more repeated enhancer nucleic acid sequences, wherein at least 30 nucleic acids separate each repeated enhancer nucleic acid sequence. In one embodiment, the liver-specific expression cassette comprises ten or more repeated enhancer nucleic acid sequences, wherein at least 30 nucleic acids separate each repeated enhancer nucleic acid sequence. In one embodiment, the liver-specific expression cassette provided herein comprises about 3 to 10 repeated enhancer nucleic acid sequences, wherein at least 30 nucleic acids separate each repeated enhancer nucleic acid sequence.

In one embodiment, the liver-specific expression cassette provided herein comprises two or more repeated enhancer nucleic acid sequences, wherein between about 2 and 30 nucleic acids separate each repeated enhancer nucleic acid sequence. In one embodiment, the liver-specific expression cassette provided herein comprises three or more repeated enhancer nucleic acid sequences, wherein between about 2 and 30 nucleic acids separate each repeated enhancer nucleic acid sequence. In one embodiment, the liver-specific expression cassette provided herein comprises five or more repeated enhancer nucleic acid sequences, wherein between about 2 and 30 nucleic acids separate each repeated enhancer nucleic acid sequence. In one embodiment, the liver-specific expression cassette provided herein comprises ten or more repeated enhancer nucleic acid sequences, wherein between about 2 and 30 nucleic acids separate each repeated enhancer nucleic acid sequence. In one embodiment, the liver-specific expression cassette comprises about 3 to 10 repeated enhancer nucleic acid sequences, wherein between about 2 and 30 nucleic acids separate each repeated enhancer nucleic acid sequence.

In one embodiment, the enhancer nucleic acid sequences are further are operably linked to a liver-specific promoter and a transgene. In one embodiment, the liver-specific promoter is a human liver-specific promoter.

In one embodiment, the liver-specific promoter is selected from the group consisting of a minimal TTR promotor (TTRm), an AAT promoter, an albumin (ALB) promotor or minimal promoter, an apolipoprotein A1 (APOA1) promoter or minimal promoter, a complement factor B (CFB) promoter, a ketohexokinase (KHK) promoter, a hemopexin (HPX) promoter or minimal promoter, a nicotinamide N-methyltransferase (NNMT) promoter or minimal promoter, a carboxylesterase 1 (CES1) promoter or minimal promoter, a protein C (PROC) promoter or minimal promoter, an apolipoprotein C3 (APOC3) promoter or minimal promoter, a mannan-binding lectin serine protease 2 (MASP2) promoter or minimal promoter, a hepcidin antimicrobial peptide (HAMP) promoter or minimal promoter, or a serpin peptidase inhibitor, clade C (antithrombin), member 1 (SERPINC1) promoter or minimal promoter.

In some embodiments, a promoter may also be a promoter from a human gene. The promoter may also be a tissue specific promoter, such as a liver-specific promoter, such as human alpha 1-antitypsin (HAAT). In one embodiment, the promoter may be synthetic.

Non-limiting examples of suitable promoters for use in accordance with the present disclosure include any of the promoters described herein, or any of the following:

In one embodiment, the promoter is hAAT core, the human a1 antitrypsin (hAAT) promoter (Core promoter sequence from human A1AT gene). In one embodiment, the hAAT promoter comprises the sequence set forth as SEQ ID NO: 210 below:

(SEQ ID NO: 210)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCCGATAACTGGG
GTGACCTTGGTTAATATTCACCAGCAGCCTCCCCCGTTGCCCCTC
TGGATCCACTGCTTAAATACGGACGAGGACAGG

In one embodiment, the promoter comprises a nucleic acid sequence at least about 85% identical to SEQ ID NO: 210. In one embodiment, the promoter comprises a nucleic acid sequence at least about 90% identical to SEQ ID NO: 210. In one embodiment, the promoter comprises a nucleic acid sequence at least about 95% identical to SEQ ID NO: 210. In one embodiment, the promoter comprises a nucleic acid sequence at least about 96% identical to SEQ ID NO: 210. In one embodiment, the promoter comprises a nucleic acid sequence at least about 97% identical to SEQ ID NO: 210. In one embodiment, the promoter comprises a nucleic acid sequence at least about 98% identical to SEQ ID NO: 210. In one embodiment, the promoter comprises a nucleic acid sequence at least about 99% identical to SEQ ID NO: 210. In one embodiment, the promoter consists of the nucleic acid sequence of SEQ ID NO: 210.

In one embodiment, the promoter is the minimal transthyretin promoter (TTRm). In one embodiment, the TTRm promoter comprises the sequence set forth as SEQ ID NO: 211 below:

(SEQ ID NO: 211)
GTCTGTCTGCACATTTCGTAGAGCGAGTGTTCCGATACTCTAATC
TCCCTAGGCAAGGTTCATATTTGTGTAGGTTACTTATTCTCCTTT
TGTTGACTAAGTCAATAATCAGAATCAGCAGGTTTGGAGTCAGCT
TGGCAGGGATCAGCAGCCTGGGTTGGAAGGAGGGGGTATAAAAGC
CCCTTCACCAGGAGAAGCCGTC

In one embodiment, the promoter comprises a nucleic acid sequence at least about 85% identical to SEQ ID NO: 211. In one embodiment, the promoter comprises a nucleic acid sequence at least about 90% identical to SEQ ID NO: 211. In one embodiment, the promoter comprises a nucleic acid sequence at least about 95% identical to SEQ ID NO: 211. In one embodiment, the promoter comprises a nucleic acid sequence at least about 96% identical to SEQ ID NO: 211. In one embodiment, the promoter comprises a nucleic acid sequence at least about 97% identical to SEQ ID NO: 211. In one embodiment, the promoter comprises a nucleic acid sequence at least about 98% identical to SEQ ID NO: 211. In one embodiment, the promoter comprises a nucleic acid sequence at least about 99% identical to SEQ ID NO: 211. In one embodiment, the promoter consists of the nucleic acid sequence of SEQ ID NO: 211.

In one embodiment, the promoter is hAAT_core_C06, a CpG minimized version of the hAAT core promoter (A1AT gene promoter). In one embodiment, the hAAT promoter comprises the sequence set forth as SEQ ID NO: 212 below:

(SEQ ID NO: 212)
GATCTTGCTACCAGTGGAACAGCCACTAAGGATTCTGCAGTGAGA
GCAGAGGGCCAGCTAAGTGGTACTCTCCCAGAGACTGTCTGACTC
ATGCCACCCCCTCCACCTTGGACACAGGACACTGTGGTTTCTGAG
CCAGGTACAATGACTCCTTTTGGTAAGTGCAGTGGAAGCTGTACA
CTGCCCAGGCAAAGTGTCCGGGCAGCGTAGGCGGGCGACTCAGAT
CCCAGCCAGTGGACTTAGCCCCTGTTTGCTCCTCCGATAACTGGG
GTGACCTTGGTTAATATTCACCAGCAGCCTCCCCCGTTGCCCCTC
TGGATCCACTGCTTAAATACGGACGAGGACAGG.

In one embodiment, the promoter comprises a nucleic acid sequence at least about 85% identical to SEQ ID NO: 212. In one embodiment, the promoter comprises a nucleic acid sequence at least about 90% identical to SEQ ID NO: 212. In one embodiment, the promoter comprises a nucleic acid sequence at least about 95% identical to SEQ ID NO: 212. In one embodiment, the promoter comprises a nucleic acid sequence at least about 96% identical to SEQ ID NO: 212. In one embodiment, the promoter comprises a nucleic acid sequence at least about 97% identical to SEQ ID NO: 212. In one embodiment, the promoter comprises a nucleic acid sequence at least about 98% identical to SEQ ID NO: 212. In one embodiment, the promoter comprises a nucleic acid sequence at least about 99% identical to SEQ ID NO: 212. In one embodiment, the promoter consists of the nucleic acid sequence of SEQ ID NO: 212.

In one embodiment, the promoter is hAAT_core_C07, a CpG minimized version of the hAAT core promoter (A1AT gene promoter). In one embodiment, the hAAT promoter comprises the sequence set forth as SEQ ID NO: 213 below:

(SEQ ID NO: 213)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCTGATAACTGGG
GTGACCTTGGTTAATATTCACCAGCAGCCTCCCCTGTTGCCCCTC
TGGATCCACTGCTTAAATACGGACAAGGACAGG

In one embodiment, the promoter comprises a nucleic acid sequence at least about 85% identical to SEQ ID NO: 213. In one embodiment, the promoter comprises a nucleic acid sequence at least about 90% identical to SEQ ID NO: 213. In one embodiment, the promoter comprises a nucleic acid sequence at least about 95% identical to SEQ ID NO: 213. In one embodiment, the promoter comprises a nucleic acid sequence at least about 96% identical to SEQ ID NO: 213. In one embodiment, the promoter comprises a nucleic acid sequence at least about 97% identical to SEQ ID NO: 213. In one embodiment, the promoter comprises a nucleic acid sequence at least about 98% identical to SEQ ID NO: 213. In one embodiment, the promoter comprises a nucleic acid sequence at least about 99% identical to SEQ ID NO: 213. In one embodiment, the promoter consists of the nucleic acid sequence of SEQ ID NO: 213.

In one embodiment, the promoter is hAAT_core_C08, a CpG minimized version of the hAAT core promoter (A1AT gene promoter). In one embodiment, the hAAT promoter comprises the sequence set forth as SEQ ID NO: 214 below:

(SEQ ID NO: 214)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCTGGGCAGCATAGGCAGGCGACTCAGAT
CCCAGCCAGTGGACTTAGCCCCTGTTTGCTCCTCCGATAACTGGG
GTGACCTTGGTTAATATTCACCAGCAGCCTCCCCCGTTGCCCCTC
TGGATCCACTGCTTAAATACGGACGAGGACAGG

In one embodiment, the promoter comprises a nucleic acid sequence at least about 85% identical to SEQ ID NO: 214. In one embodiment, the promoter comprises a nucleic acid sequence at least about 90% identical to SEQ ID NO: 214. In one embodiment, the promoter comprises a nucleic acid sequence at least about 95% identical to SEQ ID NO: 214. In one embodiment, the promoter comprises a nucleic acid sequence at least about 96% identical to SEQ ID NO: 214. In one embodiment, the promoter comprises a nucleic acid sequence at least about 97% identical to SEQ ID NO: 214. In one embodiment, the promoter comprises a nucleic acid sequence at least about 98% identical to SEQ ID NO: 214. In one embodiment, the promoter comprises a nucleic acid sequence at least about 99% identical to SEQ ID NO: 214. In one embodiment, the promoter consists of the nucleic acid sequence of SEQ ID NO: 214.

In one embodiment, the promoter is hAAT_core_C09, a CpG minimized version of the hAAT core promoter (A1AT gene promoter). In one embodiment, the hAAT promoter comprises the sequence set forth as SEQ ID NO: 215 below:

(SEQ ID NO: 215)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCTGATAACTGGG
GTGACCTTGGTTAATATTCACCAGCAGCCTCCCCCGTTGCCCCTC
TGGATCCACTGCTTAAATACAGACGAGGACAGG

In one embodiment, the promoter comprises a nucleic acid sequence at least about 85% identical to SEQ ID NO: 215. In one embodiment, the promoter comprises a nucleic acid sequence at least about 90% identical to SEQ ID NO: 215. In one embodiment, the promoter comprises a nucleic acid sequence at least about 95% identical to SEQ ID NO: 215. In one embodiment, the promoter comprises a nucleic acid sequence at least about 96% identical to SEQ ID NO: 215. In one embodiment, the promoter comprises a nucleic acid sequence at least about 97% identical to SEQ ID NO: 215. In one embodiment, the promoter comprises a nucleic acid sequence at least about 98% identical to SEQ ID NO: 215. In one embodiment, the promoter comprises a nucleic acid sequence at least about 99% identical to SEQ ID NO: 215. In one embodiment, the promoter consists of the nucleic acid sequence of SEQ ID NO: 215.

In one embodiment, the promoter is hAAT_core_C10, a CpG minimized version of the hAAT core promoter (A1AT gene promoter). In one embodiment, the hAAT promoter comprises the sequence set forth as SEQ ID NO: 216 below:

(SEQ ID NO: 216)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCTGATAACTGGG
GTGACCTTGGTTAATATTCACCAGCAGCCTCCCCCGTTGCCCCTC
TGGATCCACTGCTTAAATACAGACGAGGACAGG

In one embodiment, the promoter comprises a nucleic acid sequence at least about 85% identical to SEQ ID NO: 216. In one embodiment, the promoter comprises a nucleic acid sequence at least about 90% identical to SEQ ID NO: 216. In one embodiment, the promoter comprises a nucleic acid sequence at least about 95% identical to SEQ ID NO: 216. In one embodiment, the promoter comprises a nucleic acid sequence at least about 96% identical to SEQ ID NO: 216. In one embodiment, the promoter comprises a nucleic acid sequence at least about 97% identical to SEQ ID NO: 216. In one embodiment, the promoter comprises a nucleic acid sequence at least about 98% identical to SEQ ID NO: 216. In one embodiment, the promoter comprises a nucleic acid sequence at least about 99% identical to SEQ ID NO: 216. In one embodiment, the promoter consists of the nucleic acid sequence of SEQ ID NO: 216.

In one embodiment, the promoter is hAAT_core_truncated, 5p truncated hAAT core promoter derived from hAAT_core (SEQ ID NO: 210). In one embodiment, the hAAT promoter comprises the sequence set forth as SEQ ID NO: 217 below:

(SEQ ID NO: 217)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCTGATAACTGGG
GTGACCTTGGTTAATATTCACCAGCAGCCTCCCCCGTTGCCCCTC
TGGATCCACTGCTTAAATACAGACGAGGACAGG

In one embodiment, the promoter comprises a nucleic acid sequence at least about 85% identical to SEQ ID NO: 217. In one embodiment, the promoter comprises a nucleic acid sequence at least about 90% identical to SEQ ID NO: 217. In one embodiment, the promoter comprises a nucleic acid sequence at least about 95% identical to SEQ ID NO: 217. In one embodiment, the promoter comprises a nucleic acid sequence at least about 96% identical to SEQ ID NO: 217. In one embodiment, the promoter comprises a nucleic acid sequence at least about 97% identical to SEQ ID NO: 217. In one embodiment, the promoter comprises a nucleic acid sequence at least about 98% identical to SEQ ID NO: 217. In one embodiment, the promoter comprises a nucleic acid sequence at least about 99% identical to SEQ ID NO: 217. In one embodiment, the promoter consists of the nucleic acid sequence of SEQ ID NO: 217.

Table 1 below lists core promoter sequences, and their corresponding SEQ ID NOs, that can be implemented in ceDNA FVIII therapeutics described herein.

TABLE 1
Core Promoters

| | | | SEQ ID |
|---|---|---|---|
| | Name | Description | NO. |
| GE-015 | hAAT_core | Core promoter sequence from human A1AT | 210 |
| | | gene | |
| GE-1121 | TTRm | Core promoter sequence from mouse | 211 |
| | | Transthyretin gene | |
| GE-1133 | hAAT_core_C06 | CpG minimized version of the hAAT core | 212 |
| | | promoter (A1AT gene promoter) | |
| GE-1134 | hAAT_core_C07 | CpG minimized version of the hAAT core | 213 |
| | | promoter (A1AT gene promoter) | |
| GE-1135 | hAAT_core_C08 | CpG minimized version of the hAAT core | 214 |
| | | promoter (A1AT gene promoter) | |
| GE-1136 | hAAT_core_C09 | CpG minimized version of the hAAT core | 215 |
| | | promoter (A1AT gene promoter) | |
| GE-1137 | hAAT_core_C10 | CpG minimized version of the hAAT core | 216 |
| | | promoter (A1AT gene promoter) (also | |
| | | referred to as hAAT(979)) | |
| GE-1170 | hAAT_core_truncated | 5p truncated hAAT core promoter derived | 217 |
| | | from GE-015 | |

According to particular embodiments, the promoter is selected from the group consisting of: human alpha 1-antitrypsin (hAAT) promoter (including the CpG minimized hAAT(979) promoter (CpGmin hAAT_core_C10) and other CpGmin_hAAT promoters like hAAT_core_C06; hAAT_core_C07; hAAT_core_C08; and hAAT_core_C09) and the transthyretin (TTR) liver-specific promoter.

In one embodiment, the TTRm comprises SEQ ID NO: 211. In one embodiment, the serpin enhancer comprises SEQ ID NO: 19. In one embodiment, the TTRm 5′UTR comprises SEQ ID NO: 141 (ACACAGATCCACAAGCTCCTG).

In one embodiment, the CpGmin_hAAT promoter comprises a sequence selected from any one of SEQ ID NOs 212, 213, 214, 215 or 216.

In one embodiment, the enhancer is selected from the group consisting of: a SERPIN enhancer (SerpEnh), human SERPINA1 enhancer, Hepatic Nuclear Factor 4 binding site (HNF4), the transthyretin (TTRe) gene enhancer (TTRe), the Hepatic Nuclear Factor 1 binding site (HNF1), Human apolipoprotein E/C-I liver-specific enhancer (ApoE_Enh), the enhancer region from Pro-albumin gene (ProEnh).

In one embodiment, the enhancer is a SERPINA1 enhancer. In one embodiment, the enhancer is a SERPINA1 enhancer variant, selected from a nucleic acid sequence as set forth in Table 4, herein. In one embodiment, the SERPINA1 enhancer comprises a sequence having at lest 90%, 91%, 92%, 93%, 94%, 95%, 96%, 97%, 98%, 99% identity to, comprises or consists of any one of the nucleic acid sequences set forth in Table 2, herein.

According to further embodiments, the enhancer is a human SERPIN1A enhancer. According to still further embodiments, the human SERPIN1A enhancer comprises SEQ ID NO: 81 shown below.

SEQ ID NO: 81
(SEQ ID NO: 81)
GGGGGAGGCTGCTGGTGAATATTAACCAAGGTCACCCCAGTTATC
GGAGGAGCAAACAGGGGCTAAGTCCAC

In one embodiment, the enhancer is a Chinese Tree Shrew SERPINA1 enhancer. According to further embodiments, the Chinese Tree Shrew SERPINA1 enhancer comprises SEQ ID NO: 82 shown below.

(SEQ ID NO: 82)
GGAGGCTGTTGGTGAATATTAACCAAGGTCACCCCAGTTATCGGA
GGAGCAAACAAGGGCTAAGTCCAC

In one embodiment, the enhancer is a Chinese Tree Shrew SERPINA1 enhancer. According to further embodiments, the Chinese Tree Shrew SERPINA1 enhancer comprises SEQ ID NO: 122 shown below.

(SEQ ID NO: 122)
GGAGGCTGTTGGTGAATATTAACCAAGGTCACCTCAGTTATCGGA
GGAGCAAACAAGGGCTAAGTCCAC

In one embodiment, the enhancer is a Bushbaby SERPINA1 enhancer. According to further embodiments, the Bushbaby SERPINA1 enhancer comprises SEQ ID NO: 83 shown below.

(SEQ ID NO: 83)
GGGGGAAGCTACTGGTGAATATTAACCAAGGTCACCCAGTTATCA
GGGAGCAAACAGGAGCTAAGTCCAT

In one embodiment, the enhancer is a HNF4 enhancer. In one embodiment, the enhancer is HNF4. According to further embodiments, the HNF4 enhancer comprises SEQ ID NO: 84 shown below.

(SEQ ID NO: 84)
GAGGGAGGCTGCTGGTGAATATTAACCAAGGTCACCTCAGTTATC
AGAGGAGCAAACAGGGGCAAAGTCCAT

In one embodiment, the enhancer is HNF4_FOXA. According to further embodiments, the HNF4_FOXA enhancer comprises SEQ ID NO: 85 shown below.

(SEQ ID NO: 85)
GGGGGAGGCTGCTGGTAAACATTAACCAAGGTCACCCCAGTTATC
AGAGGAGCAAACAGGGGCAAAGTCCAC

CpG dinucleotides are undesirable for gene therapy applications. CpGs can impact expression durability through stimulation of the innate immune system and through methylation-based silencing. Accordingly, in some embodiments, CpGs are removed from the enhancer nucleic acid sequences. In one embodiment, internal CpGs are removed.

In one embodiment, the enhancer comprises human SERPINA1 enhancer, wherein CpG dinucleotides have been minimized.

In one embodiment, the enhancer comprises Chinese Tree Shrew SERPINA1 enhancer, wherein CpG dinucleotides have been minimized.

In one embodiment, the enhancer comprises Bushbaby SERPINA1 enhancer, wherein CpG dinucleotides have been minimized.

In one embodiment, the enhancer comprises HNF4, wherein CpG dinucleotides have been minimized.

In one embodiment, the enhancer comprises HNF4_FOXA, wherein CpG dinucleotides have been minimized.

In one embodiment, the enhancer comprises human SERPINA1 enhancer, wherein poly-C/poly-G have been minimized.

In one embodiment, the enhancer comprises Chinese Tree Shrew SERPINA1 enhancer, wherein poly-C/poly-G have been minimized.

In one embodiment, the enhancer comprises Bushbaby SERPINA1 enhancer, wherein poly-C/poly-G have been minimized.

In one embodiment, the enhancer comprises HNF4, wherein poly-C/poly-G have been minimized.

In one embodiment, the enhancer comprises HNF4_FOXA, wherein poly-C/poly-G have been minimized.

In one embodiment, the enhancer comprises human SERPINA enhancer, wherein CpG dinucleotides and poly-C/poly-G have been minimized.

In one embodiment, the enhancer comprises Chinese Tree Shrew SERPINA1 enhancer, wherein CpG dinucleotides and poly-C/poly-G have been minimized.

In one embodiment, the enhancer comprises Bushbaby SERPINA1 enhancer, wherein CpG dinucleotides and poly-C/poly-G have been minimized.

In one embodiment, the enhancer comprises HNF4, wherein CpG dinucleotides and poly-C/poly-G have been minimized.

In one embodiment, the enhancer comprises HNF4_FOXA, wherein CpG dinucleotides and poly-C/poly-G have been minimized.

In some embodiments, the enhancer is selected from a sequence shown in Table 2, below.

TABLE 2
Enhancers

| | | | SEQ |
|---|---|---|---|
| | | | ID |
| Name | Description | Sequence | NO: |
| 3x_HNF4 | 3× repeat of the | GGGGGAGGCTGCTGGTAAACATTAACCAAGGT | 1 |
| FOXA_v1 | Human SERPINA1 | CACCCCAGTTATCAGAGGAGCAAACAGGGGCA | |
| | enhancer with FOXA | AAGTCCACCGGGGGAGGCTGCTGGTAAACATT | |
| | & HNF4 consensus | AACCAAGGTCACCCCAGTTATCAGAGGAGCAA | |
| | sites (“C” spacer in | ACAGGGGCAAAGTCCACCGGGGGAGGCTGCTG | |
| | bold) | GTAAACATTAACCAAGGTCACCCCAGTTATCA | |
| | | GAGGAGCAAACAGGGGCAAAGTCCAC | |
| 3x_HNF4 | 3× repeat of | AGGGGAGGCTGCTGGTAAACATTAACCAAGGT | 2 |
| FOXA_v1 | HNF4_FOXA_v1 | CACCCCAGTTATCAGAGGAGCAAACAGGGGCA | |
| CpGmin | with CpG | AAGTCCACAGGGGGAGGCTGCTGGTAAACATT | |
| | minimization (“A” | AACCAAGGTCACCCCAGTTATCAGAGGAGCAA | |
| | spacer in bold) | ACAGGGGCAAAGTCCACAGGGGGAGGCTGCTG | |
| | | GTAAACATTAACCAAGGTCACCCCAGTTATCA | |
| | | GAGGAGCAAACAGGGGCAAAGTCCAT | |
| 3x_HNF4 | 3× repeat of | GAGGGAGGCTGCTGGTAAACATTAACCAAGGT | 3 |
| FOXA_v1 | HNF4_FOXA_v1 | CACCCAGTTATCAGAGGAGCAAACAGGGGCAA | |
| Secondary | with poly-C/poly-G | AGTCCACCGAGGGAGGCTGCTGGTAAACATTA | |
| Struct_min_ | minimization v1 (“C” | ACCAAGGTCACCCAGTTATCAGAGGAGCAAAC | |
| v1 | spacer in bold) | AGGGGCAAAGTCCACCGAGGGAGGCTGCTGGT | |
| | | AAACATTAACCAAGGTCACCCAGTTATCAGAG | |
| | | GAGCAAACAGGGGCAAAGTCCAC | |
| 3x_HNF4 | 3× repeat of | AGGGAGGCTGCTGGTAAACATTAACCAAGGTC | 4 |
| FOXA_v1 | HNF4_FOXA_v1 | ACCCAGTTATCAGAGGAGCAAACAGGGGCAAA | |
| Secondary | with poly-C/poly-G | GTCCACAGAGGGAGGCTGCTGGTAAACATTAA | |
| Struct_min_ | minimization and | CCAAGGTCACCCAGTTATCAGAGGAGCAAACA | |
| v1_CpG_min | CpG minimization v1 | GGGGCAAAGTCCACAGAGGGAGGCTGCTGGTA | |
| | (“A” spacer in bold) | AACATTAACCAAGGTCACCCAGTTATCAGAGG | |
| | | AGCAAACAGGGGCAAAGTCCAT | |
| 3x_HNF4 | 3× repeat of | GGGGGAGGCTGCTGGTAAACATTAACCAAGGT | 5 |
| FOXA_v1 | HNF4_FOXA_v1 | CACCTCAGTTATCAGAGGAGCAAACAGGGACA | |
| Secondary | with poly-C/poly-G | AAGTCCACCGGGGGAGGCTGCTGGTAAACATT | |
| Struct_min_ | minimization v2 (“C” | AACCAAGGTCACCTCAGTTATCAGAGGAGCAA | |
| v2 | spacer) | ACAGGGACAAAGTCCACCGGGGGAGGCTGCTG | |
| | | GTAAACATTAACCAAGGTCACCTCAGTTATCAG | |
| | | AGGAGCAAACAGGGACAAAGTCCAC | |
| 3x_HNF4 | 3× repeat of | AGGGGAGGCTGCTGGTAAACATTAACCAAGGT | 6 |
| FOXA_v1 | HNF4_FOXA_v1 | CACCTCAGTTATCAGAGGAGCAAACAGGGACA | |
| Secondary | with poly-C/poly-G | AAGTCCACAGGGGGAGGCTGCTGGTAAACATT | |
| Struct_min_ | minimization and | AACCAAGGTCACCTCAGTTATCAGAGGAGCAA | |
| v2_CpG_min | CpG minimization v2 | ACAGGGACAAAGTCCACAGGGGGAGGCTGCTG | |
| | (“A” spacer) | GTAAACATTAACCAAGGTCACCTCAGTTATCAG | |
| | | AGGAGCAAACAGGGACAAAGTCCACA | |
| 3x_HNF4 | 3× repeat of | GGGAGGCTGCTGGTAAACATTAACCAAGGTCA | 7 |
| FOXA_v1 | HNF4_FOXA_v1 | CCCCAGTTATCAGAGGAGCAAACAAGGGCAAA | |
| Secondary | with poly-C/poly-G | GTCCACCGGGAGGCTGCTGGTAAACATTAACC | |
| Struct_min_ | minimization v3 (“C” | AAGGTCACCCCAGTTATCAGAGGAGCAAACAA | |
| v3 | spacer) | GGGCAAAGTCCACCGGGAGGCTGCTGGTAAAC | |
| | | ATTAACCAAGGTCACCCCAGTTATCAGAGGAG | |
| | | CAAACAAGGGCAAAGTCCAC | |
| 3x_HNF4 | 3× repeat of | AGGGAGGCTGCTGGTAAACATTAACCAAGGTC | 8 |
| FOXA_v1 | HNF4_FOXA_v1 | ACCCCAGTTATCAGAGGAGCAAACAAGGGCAA | |
| Secondary | with poly-C/poly-G | AGTCCACAGGGAGGCTGCTGGTAAACATTAAC | |
| Struct_min_ | minimization and | CAAGGTCACCCCAGTTATCAGAGGAGCAAACA | |
| v3_CpG_min | CpG minimization v3 | AGGGCAAAGTCCACAGGGAGGCTGCTGGTAAA | |
| | (“A” spacer) | CATTAACCAAGGTCACCCCAGTTATCAGAGGA | |
| | | GCAAACAAGGGCAAAGTCCACA | |
| 3x_HNF4 | 3× repeat of | AGGAGGAGGCTGCTGGTAAACATTAACCAAGG | 9 |
| FOXA_v1 | HNF4_FOXA_v1 | TCACCTCAGTTATCAGAGGAGCAAACAGGGGC | |
| Secondary | with poly-C/poly-G | AAAGTCCACAGGAGGAGGCTGCTGGTAAACAT | |
| Struct_min_ | minimization v4 | TAACCAAGGTCACCTCAGTTATCAGAGGAGCA | |
| v4_Aspacers | (2585) | AACAGGGGCAAAGTCCACAGGAGGAGGCTGCT | |
| (no spacer | | GGTAAACATTAACCAAGGTCACCTCAGTTATCA | |
| inbetween | | GAGGAGCAAACAGGGGCAAAGTCCACA | |
| the repeats) | | | |
| 3x_HNF4 | 3× repeat of | AGGGGGAGGCTGCTGGTAAACATTAACCAAGG | 10 |
| FOXA_v1 | HNF4_FOXA_v1 | TCACCTCAGTTATCAGAGGAGCAAACAGGTGC | |
| Secondary | with poly-C/poly-G | AAAGTCCACAGGGGGAGGCTGCTGGTAAACAT | |
| Struct_min_ | minimization v5 | TAACCAAGGTCACCTCAGTTATCAGAGGAGCA | |
| v5_Aspacers | | AACAGGTGCAAAGTCCACAGGGGGAGGCTGCT | |
| (“A” spacer | | GGTAAACATTAACCAAGGTCACCTCAGTTATCA | |
| inbetween | | GAGGAGCAAACAGGTGCAAAGTCCACA | |
| the repeats) | | | |
| 3x_HNF4 | 3× repeat of | AGGAGGAGGCTGCTGGTAAACATTAACCAAGG | 11 |
| FOXA_v1 | HNF4_FOXA_v1 | TCACCCCAGTTATCAGAGGAGCAAACAGGTGC | |
| Secondary | with poly-C/poly-G | AAAGTCCACAGGAGGAGGCTGCTGGTAAACAT | |
| Struct_min_ | minimization v6 | TAACCAAGGTCACCCCAGTTATCAGAGGAGCA | |
| v6_Aspacers | | AACAGGTGCAAAGTCCACAGGAGGAGGCTGCT | |
| (“A” spacer | | GGTAAACATTAACCAAGGTCACCCCAGTTATC | |
| inbetween | | AGAGGAGCAAACAGGTGCAAAGTCCACA | |
| the repeats) | | | |
| 3x_Chinese | 3× repeat of the | GGAGGCTGTTGGTGAATATTAACCAAGGTCAC | 12 |
| TreeShrew | Chinese Tree Shrew | CTCAGTTATCGGAGGAGCAAACAAGGGCTAAG | |
| | SERPINA1 enhancer | TCCACCGGAGGCTGTTGGTGAATATTAACCAA | |
| | (“C” spancer | GGTCACCTCAGTTATCGGAGGAGCAAACAAGG | |
| | inbetween the | GCTAAGTCCACCGGAGGCTGTTGGTGAATATT | |
| | repeats) | AACCAAGGTCACCTCAGTTATCGGAGGAGCAA | |
| | | ACAAGGGCTAAGTCCAC | |
| 3x_Chinese | 3× repeat of the | AGGAGGCTGTTGGTGAATATTAACCAAGGTCA | 13 |
| TreeShrew | Chinese Tree Shrew | CCTCAGTTATCAGAGGAGCAAACAAGGGCTAA | |
| CpGmin | SERPINA1 enhancer | GTCCACAGGAGGCTGTTGGTGAATATTAACCA | |
| | with CpG | AGGTCACCTCAGTTATCAGAGGAGCAAACAAG | |
| | minimization (no | GGCTAAGTCCACAGGAGGCTGTTGGTGAATAT | |
| | spacer) | TAACCAAGGTCACCTCAGTTATCAGAGGAGCA | |
| | | AACAAGGGCTAAGTCCACA | |
| 3x_hSerpEn | 3× repeat of the | GGGGGAGGCTGCTGGTGAATATTAACCAAGGT | 14 |
| h_Aspacers | human SERPINA1 | CACCCCAGTTATCGGAGGAGCAAACAGGGGCT | |
| | enhancer with 1 | AAGTCCACAGGGGGAGGCTGCTGGTGAATATT | |
| | adenine between | AACCAAGGTCACCCCAGTTATCGGAGGAGCAA | |
| | repeats (“A” spacer) | ACAGGGGCTAAGTCCACAGGGGGAGGCTGCTG | |
| | | GTGAATATTAACCAAGGTCACCCCAGTTATCGG | |
| | | AGGAGCAAACAGGGGCTAAGTCCAC | |
| 3x_Bushba | 3× repeat of the | AGGGGAAGCTACTGGTGAATATTAACCAAGGT | 15 |
| by_Aspacers | Bushbaby | CACCCAGTTATCAGGGAGCAAACAGGAGCTAA | |
| | SERPINA1 enhancer | GTCCATAGGGGGAAGCTACTGGTGAATATTAA | |
| | with adenine | CCAAGGTCACCCAGTTATCAGGGAGCAAACAG | |
| | nucleotide spacer (no | GAGCTAAGTCCATAGGGGGAAGCTACTGGTGA | |
| | spacer) | ATATTAACCAAGGTCACCCAGTTATCAGGGAG | |
| | | CAAACAGGAGCTAAGTCCAT | |
| 5x_HNF4 | 5× repeat of | GGGGGAGGCTGCTGGTAAACATTAACCAAGGT | 16 |
| FOXA_v1 | HNF4_FOXA_v1 | CACCCCAGTTATCAGAGGAGCAAACAGGGGCA | |
| | (“C” spacer) | AAGTCCACCGGGGGAGGCTGCTGGTAAACATT | |
| | | AACCAAGGTCACCCCAGTTATCAGAGGAGCAA | |
| | | ACAGGGGCAAAGTCCACCGGGGGAGGCTGCTG | |
| | | GTAAACATTAACCAAGGTCACCCCAGTTATCA | |
| | | GAGGAGCAAACAGGGGCAAAGTCCACCGGGG | |
| | | GAGGCTGCTGGTAAACATTAACCAAGGTCACC | |
| | | CCAGTTATCAGAGGAGCAAACAGGGGCAAAGT | |
| | | CCACCGGGGGAGGCTGCTGGTAAACATTAACC | |
| | | AAGGTCACCCCAGTTATCAGAGGAGCAAACAG | |
| | | GGGCAAAGTCCAC | |
| 5x_HNF4 | 5× repeat of | GAGGGAGGCTGCTGGTAAACATTAACCAAGGT | 17 |
| FOXA_v1 | HNF4_FOXA_v1 | CACCCAGTTATCAGAGGAGCAAACAGGGGCAA | |
| Secondary | with poly-C/poly-G | AGTCCACCGAGGGAGGCTGCTGGTAAACATTA | |
| Struct_min_ | minimization v1 (“C” | ACCAAGGTCACCCAGTTATCAGAGGAGCAAAC | |
| v1 | spacer) | AGGGGCAAAGTCCACCGAGGGAGGCTGCTGGT | |
| | | AAACATTAACCAAGGTCACCCAGTTATCAGAG | |
| | | GAGCAAACAGGGGCAAAGTCCACCGAGGGAG | |
| | | GCTGCTGGTAAACATTAACCAAGGTCACCCAG | |
| | | TTATCAGAGGAGCAAACAGGGGCAAAGTCCAC | |
| | | CGAGGGAGGCTGCTGGTAAACATTAACCAAGG | |
| | | TCACCCAGTTATCAGAGGAGCAAACAGGGGCA | |
| | | AAGTCCAC | |
| 5x_HNF4 | 5× repeat of | AGGGAGGCTGCTGGTAAACATTAACCAAGGTC | 18 |
| FOXA_v1 | HNF4_FOXA_v1 | ACCCAGTTATCAGAGGAGCAAACAGGGGCAAA | |
| Secondary | with poly-C/poly-G | GTCCACAGAGGGAGGCTGCTGGTAAACATTAA | |
| Struct_min_ | minimization and | CCAAGGTCACCCAGTTATCAGAGGAGCAAACA | |
| v1_CpG_min | CpG minimization v1 | GGGGCAAAGTCCACAGAGGGAGGCTGCTGGTA | |
| | (″AG″ spacer) | AACATTAACCAAGGTCACCCAGTTATCAGAGG | |
| | | AGCAAACAGGGGCAAAGTCCACAGAGGGAGG | |
| | | CTGCTGGTAAACATTAACCAAGGTCACCCAGTT | |
| | | ATCAGAGGAGCAAACAGGGGCAAAGTCCACA | |
| | | GAGGGAGGCTGCTGGTAAACATTAACCAAGGT | |
| | | CACCCAGTTATCAGAGGAGCAAACAGGGGCAA | |
| | | AGTCCAT | |
| 5x_HNF4 | 5× repeat of | GGGGGAGGCTGCTGGTAAACATTAACCAAGGT | 19 |
| FOXA_v1 | HNF4_FOXA_v1 | CACCTCAGTTATCAGAGGAGCAAACAGGGACA | |
| Secondary | with poly-C/poly-G | AAGTCCACCGGGGGAGGCTGCTGGTAAACATT | |
| Struct_min_ | minimization v2 (“C” | AACCAAGGTCACCTCAGTTATCAGAGGAGCAA | |
| v2 | spacer) | ACAGGGACAAAGTCCACCGGGGGAGGCTGCTG | |
| | | GTAAACATTAACCAAGGTCACCTCAGTTATCAG | |
| | | AGGAGCAAACAGGGACAAAGTCCACCGGGGG | |
| | | AGGCTGCTGGTAAACATTAACCAAGGTCACCT | |
| | | CAGTTATCAGAGGAGCAAACAGGGACAAAGTC | |
| | | CACCGGGGGAGGCTGCTGGTAAACATTAACCA | |
| | | AGGTCACCTCAGTTATCAGAGGAGCAAACAGG | |
| | | GACAAAGTCCAC | |
| 5x_HNF4 | 5× repeat of | AGGGGAGGCTGCTGGTAAACATTAACCAAGGT | 20 |
| FOXA_v1 | HNF4_FOXA_v1 | CACCTCAGTTATCAGAGGAGCAAACAGGGACA | |
| Secondary | with poly-C/poly-G | AAGTCCACAGGGGGAGGCTGCTGGTAAACATT | |
| Struct_min_ | minimization and | AACCAAGGTCACCTCAGTTATCAGAGGAGCAA | |
| v2_CpG_min | CpG minimization v2 | ACAGGGACAAAGTCCACAGGGGGAGGCTGCTG | |
| | (“A” spacer) | GTAAACATTAACCAAGGTCACCTCAGTTATCAG | |
| | | AGGAGCAAACAGGGACAAAGTCCACAGGGGG | |
| | | AGGCTGCTGGTAAACATTAACCAAGGTCACCT | |
| | | CAGTTATCAGAGGAGCAAACAGGGACAAAGTC | |
| | | CACAGGGGGAGGCTGCTGGTAAACATTAACCA | |
| | | AGGTCACCTCAGTTATCAGAGGAGCAAACAGG | |
| | | GACAAAGTCCACA | |
| 5x_HNF4 | 5× repeat of | GGGAGGCTGCTGGTAAACATTAACCAAGGTCA | 21 |
| FOXA_v1 | HNF4_FOXA_v1 | CCCCAGTTATCAGAGGAGCAAACAAGGGCAAA | |
| Secondary | with poly-C/poly-G | GTCCACCGGGAGGCTGCTGGTAAACATTAACC | |
| Struct_min_ | minimization v3 (“C” | AAGGTCACCCCAGTTATCAGAGGAGCAAACAA | |
| v3 | spacer) | GGGCAAAGTCCACCGGGAGGCTGCTGGTAAAC | |
| | | ATTAACCAAGGTCACCCCAGTTATCAGAGGAG | |
| | | CAAACAAGGGCAAAGTCCACCGGGAGGCTGCT | |
| | | GGTAAACATTAACCAAGGTCACCCCAGTTATC | |
| | | AGAGGAGCAAACAAGGGCAAAGTCCACCGGG | |
| | | AGGCTGCTGGTAAACATTAACCAAGGTCACCC | |
| | | CAGTTATCAGAGGAGCAAACAAGGGCAAAGTC | |
| | | CAC | |
| 5x_HNF4 | 5× repeat of | AGGGAGGCTGCTGGTAAACATTAACCAAGGTC | 22 |
| FOXA_v1 | HNF4_FOXA_v1 | ACCCCAGTTATCAGAGGAGCAAACAAGGGCAA | |
| Secondary | with poly-C/poly-G | AGTCCACAGGGAGGCTGCTGGTAAACATTAAC | |
| Struct_min_ | minimization and | CAAGGTCACCCCAGTTATCAGAGGAGCAAACA | |
| v3_CpG_min | CpG minimization v3 | AGGGCAAAGTCCACAGGGAGGCTGCTGGTAAA | |
| | | CATTAACCAAGGTCACCCCAGTTATCAGAGGA | |
| | | GCAAACAAGGGCAAAGTCCACAGGGAGGCTGC | |
| | | TGGTAAACATTAACCAAGGTCACCCCAGTTATC | |
| | | AGAGGAGCAAACAAGGGCAAAGTCCACAGGG | |
| | | AGGCTGCTGGTAAACATTAACCAAGGTCACCC | |
| | | CAGTTATCAGAGGAGCAAACAAGGGCAAAGTC | |
| | | CACA | |
| 5x_HNF4 | 5× repeat of | AGGAGGAGGCTGCTGGTAAACATTAACCAAGG | 23 |
| FOXA_v1 | HNF4_FOXA_v1 | TCACCTCAGTTATCAGAGGAGCAAACAGGGGC | |
| Secondary | with poly-C/poly-G | AAAGTCCACAGGAGGAGGCTGCTGGTAAACAT | |
| Struct_min_ | minimization v4 | TAACCAAGGTCACCTCAGTTATCAGAGGAGCA | |
| v4_Aspacers | | AACAGGGGCAAAGTCCACAGGAGGAGGCTGCT | |
| | | GGTAAACATTAACCAAGGTCACCTCAGTTATCA | |
| | | GAGGAGCAAACAGGGGCAAAGTCCACAGGAG | |
| | | GAGGCTGCTGGTAAACATTAACCAAGGTCACC | |
| | | TCAGTTATCAGAGGAGCAAACAGGGGCAAAGT | |
| | | CCACAGGAGGAGGCTGCTGGTAAACATTAACC | |
| | | AAGGTCACCTCAGTTATCAGAGGAGCAAACAG | |
| | | GGGCAAAGTCCACA | |
| 5x_HNF4 | 5× repeat of | AGGGGGAGGCTGCTGGTAAACATTAACCAAGG | 24 |
| FOXA_v1 | HNF4_FOXA_v1 | TCACCTCAGTTATCAGAGGAGCAAACAGGTGC | |
| Secondary | with poly-C/poly-G | AAAGTCCACAGGGGGAGGCTGCTGGTAAACAT | |
| Struct_min_ | minimization v5 | TAACCAAGGTCACCTCAGTTATCAGAGGAGCA | |
| v5_Aspacers | | AACAGGTGCAAAGTCCACAGGGGGAGGCTGCT | |
| | | GGTAAACATTAACCAAGGTCACCTCAGTTATCA | |
| | | GAGGAGCAAACAGGTGCAAAGTCCACAGGGG | |
| | | GAGGCTGCTGGTAAACATTAACCAAGGTCACC | |
| | | TCAGTTATCAGAGGAGCAAACAGGTGCAAAGT | |
| | | CCACAGGGGGAGGCTGCTGGTAAACATTAACC | |
| | | AAGGTCACCTCAGTTATCAGAGGAGCAAACAG | |
| | | GTGCAAAGTCCACA | |
| 5x_HNF4 | 5× repeat of | AGGAGGAGGCTGCTGGTAAACATTAACCAAGG | 25 |
| FOXA_v1 | HNF4_FOXA_v1 | TCACCCCAGTTATCAGAGGAGCAAACAGGTGC | |
| Secondary | with poly-C/poly-G | AAAGTCCACAGGAGGAGGCTGCTGGTAAACAT | |
| Struct_min_ | minimization v6 | TAACCAAGGTCACCCCAGTTATCAGAGGAGCA | |
| v6_Aspacers | | AACAGGTGCAAAGTCCACAGGAGGAGGCTGCT | |
| | | GGTAAACATTAACCAAGGTCACCCCAGTTATC | |
| | | AGAGGAGCAAACAGGTGCAAAGTCCACAGGA | |
| | | GGAGGCTGCTGGTAAACATTAACCAAGGTCAC | |
| | | CCCAGTTATCAGAGGAGCAAACAGGTGCAAAG | |
| | | TCCACAGGAGGAGGCTGCTGGTAAACATTAAC | |
| | | CAAGGTCACCCCAGTTATCAGAGGAGCAAACA | |
| | | GGTGCAAAGTCCACA | |
| 5x_Chinese | 5× repeat of the | GGAGGCTGTTGGTGAATATTAACCAAGGTCAC | 26 |
| TreeShrew | Chinese Tree Shrew | CTCAGTTATCGGAGGAGCAAACAAGGGCTAAG | |
| | SERPINA1 enhancer | TCCACCGGAGGCTGTTGGTGAATATTAACCAA | |
| | | GGTCACCTCAGTTATCGGAGGAGCAAACAAGG | |
| | | GCTAAGTCCACCGGAGGCTGTTGGTGAATATTA | |
| | | ACCAAGGTCACCTCAGTTATCGGAGGAGCAAA | |
| | | CAAGGGCTAAGTCCACCGGAGGCTGTTGGTGA | |
| | | ATATTAACCAAGGTCACCTCAGTTATCGGAGG | |
| | | AGCAAACAAGGGCTAAGTCCACCGGAGGCTGT | |
| | | TGGTGAATATTAACCAAGGTCACCTCAGTTATC | |
| | | GGAGGAGCAAACAAGGGCTAAGTCCAC | |
| 5x_Chinese | 5× repeat of the | AGGAGGCTGTTGGTGAATATTAACCAAGGTCA | 27 |
| TreeShrew | Chinese Tree Shrew | CCTCAGTTATCAGAGGAGCAAACAAGGGCTAA | |
| CpGmin | SERPINA1 enhancer | GTCCACAGGAGGCTGTTGGTGAATATTAACCA | |
| | with CpG | AGGTCACCTCAGTTATCAGAGGAGCAAACAAG | |
| | minimization | GGCTAAGTCCACAGGAGGCTGTTGGTGAATAT | |
| | | TAACCAAGGTCACCTCAGTTATCAGAGGAGCA | |
| | | AACAAGGGCTAAGTCCACAGGAGGCTGTTGGT | |
| | | GAATATTAACCAAGGTCACCTCAGTTATCAGA | |
| | | GGAGCAAACAAGGGCTAAGTCCACAGGAGGCT | |
| | | GTTGGTGAATATTAACCAAGGTCACCTCAGTTA | |
| | | TCAGAGGAGCAAACAAGGGCTAAGTCCACA | |
| 5x_Bushba | 5× repeat of the | AGGGGAAGCTACTGGTGAATATTAACCAAGGT | 28 |
| by_Aspacers | Bushbaby | CACCCAGTTATCAGGGAGCAAACAGGAGCTAA | |
| | SERPINA1 enhancer | GTCCATAGGGGGAAGCTACTGGTGAATATTAA | |
| | with adenenine | CCAAGGTCACCCAGTTATCAGGGAGCAAACAG | |
| | nucleotide spacer | GAGCTAAGTCCATAGGGGGAAGCTACTGGTGA | |
| | | ATATTAACCAAGGTCACCCAGTTATCAGGGAG | |
| | | CAAACAGGAGCTAAGTCCATAGGGGGAAGCTA | |
| | | CTGGTGAATATTAACCAAGGTCACCCAGTTATC | |
| | | AGGGAGCAAACAGGAGCTAAGTCCATAGGGGG | |
| | | AAGCTACTGGTGAATATTAACCAAGGTCACCC | |
| | | AGTTATCAGGGAGCAAACAGGAGCTAAGTCCA | |
| | | T | |
| 5x_hSerpEnh | 5× repeat of the | GGGGGAGGCTGCTGGTGAATATTAACCAAGGT | 29 |
| | human SERPINA1 | CACCCCAGTTATCGGAGGAGCAAACAGGGGCT | |
| | enhancer | AAGTCCACCGGGGGAGGCTGCTGGTGAATATT | |
| | | AACCAAGGTCACCCCAGTTATCGGAGGAGCAA | |
| | | ACAGGGGCTAAGTCCACCGGGGGAGGCTGCTG | |
| | | GTGAATATTAACCAAGGTCACCCCAGTTATCGG | |
| | | AGGAGCAAACAGGGGCTAAGTCCACCGGGGGA | |
| | | GGCTGCTGGTGAATATTAACCAAGGTCACCCC | |
| | | AGTTATCGGAGGAGCAAACAGGGGCTAAGTCC | |
| | | ACCGGGGGAGGCTGCTGGTGAATATTAACCAA | |
| | | GGTCACCCCAGTTATCGGAGGAGCAAACAGGG | |
| | | GCTAAGTCCAC | |
| 10x_HNF4_ | 10× repeat of | GGGGGAGGCTGCTGGTAAACATTAACCAAGGT | 30 |
| FOXA_v1 | HNF4_FOXA_v1 | CACCCCAGTTATCAGAGGAGCAAACAGGGGCA | |
| | | AAGTCCACCGGGGGAGGCTGCTGGTAAACATT | |
| | | AACCAAGGTCACCCCAGTTATCAGAGGAGCAA | |
| | | ACAGGGGCAAAGTCCACCGGGGGAGGCTGCTG | |
| | | GTAAACATTAACCAAGGTCACCCCAGTTATCA | |
| | | GAGGAGCAAACAGGGGCAAAGTCCACCGGGG | |
| | | GAGGCTGCTGGTAAACATTAACCAAGGTCACC | |
| | | CCAGTTATCAGAGGAGCAAACAGGGGCAAAGT | |
| | | CCACCGGGGGAGGCTGCTGGTAAACATTAACC | |
| | | AAGGTCACCCCAGTTATCAGAGGAGCAAACAG | |
| | | GGGCAAAGTCCACCGGGGGAGGCTGCTGGTAA | |
| | | ACATTAACCAAGGTCACCCCAGTTATCAGAGG | |
| | | AGCAAACAGGGGCAAAGTCCACCGGGGGAGG | |
| | | CTGCTGGTAAACATTAACCAAGGTCACCCCAGT | |
| | | TATCAGAGGAGCAAACAGGGGCAAAGTCCACC | |
| | | GGGGGAGGCTGCTGGTAAACATTAACCAAGGT | |
| | | CACCCCAGTTATCAGAGGAGCAAACAGGGGCA | |
| | | AAGTCCACCGGGGGAGGCTGCTGGTAAACATT | |
| | | AACCAAGGTCACCCCAGTTATCAGAGGAGCAA | |
| | | ACAGGGGCAAAGTCCACCGGGGGAGGCTGCTG | |
| | | GTAAACATTAACCAAGGTCACCCCAGTTATCA | |
| | | GAGGAGCAAACAGGGGCAAAGTCCAC | |
| 10x_HNF4_ | 10× repeat of | GAGGGAGGCTGCTGGTAAACATTAACCAAGGT | 31 |
| _FOXA_v1_ | HNF4_FOXA_v1 | CACCCAGTTATCAGAGGAGCAAACAGGGGCAA | |
| Secondary | with poly-C/poly-G | AGTCCACCGAGGGAGGCTGCTGGTAAACATTA | |
| Struct_ | minimization v1 | ACCAAGGTCACCCAGTTATCAGAGGAGCAAAC | |
| min_v1 | | AGGGGCAAAGTCCACCGAGGGAGGCTGCTGGT | |
| | | AAACATTAACCAAGGTCACCCAGTTATCAGAG | |
| | | GAGCAAACAGGGGCAAAGTCCACCGAGGGAG | |
| | | GCTGCTGGTAAACATTAACCAAGGTCACCCAG | |
| | | TTATCAGAGGAGCAAACAGGGGCAAAGTCCAC | |
| | | CGAGGGAGGCTGCTGGTAAACATTAACCAAGG | |
| | | TCACCCAGTTATCAGAGGAGCAAACAGGGGCA | |
| | | AAGTCCACCGAGGGAGGCTGCTGGTAAACATT | |
| | | AACCAAGGTCACCCAGTTATCAGAGGAGCAAA | |
| | | CAGGGGCAAAGTCCACCGAGGGAGGCTGCTGG | |
| | | TAAACATTAACCAAGGTCACCCAGTTATCAGA | |
| | | GGAGCAAACAGGGGCAAAGTCCACCGAGGGA | |
| | | GGCTGCTGGTAAACATTAACCAAGGTCACCCA | |
| | | GTTATCAGAGGAGCAAACAGGGGCAAAGTCCA | |
| | | CCGAGGGAGGCTGCTGGTAAACATTAACCAAG | |
| | | GTCACCCAGTTATCAGAGGAGCAAACAGGGGC | |
| | | AAAGTCCACCGAGGGAGGCTGCTGGTAAACAT | |
| | | TAACCAAGGTCACCCAGTTATCAGAGGAGCAA | |
| | | ACAGGGGCAAAGTCCAC | |
| 10x_HNF4_ | 10× repeat of | AGGGAGGCTGCTGGTAAACATTAACCAAGGTC | |
| FOXA_v1_ | HNF4_FOXA_v1 | ACCCAGTTATCAGAGGAGCAAACAGGGGCAAA | |
| Secondary | with poly-C/poly-G | GTCCACAGAGGGAGGCTGCTGGTAAACATTAA | |
| Struct_min_ | minimization and | CCAAGGTCACCCAGTTATCAGAGGAGCAAACA | 32 |
| v1_CpG_min | CpG minimization v1 | GGGGCAAAGTCCACAGAGGGAGGCTGCTGGTA | |
| | | AACATTAACCAAGGTCACCCAGTTATCAGAGG | |
| | | AGCAAACAGGGGCAAAGTCCACAGAGGGAGG | |
| | | CTGCTGGTAAACATTAACCAAGGTCACCCAGTT | |
| | | ATCAGAGGAGCAAACAGGGGCAAAGTCCACAG | |
| | | AGGGAGGCTGCTGGTAAACATTAACCAAGGTC | |
| | | ACCCAGTTATCAGAGGAGCAAACAGGGGCAAA | |
| | | GTCCACAGAGGGAGGCTGCTGGTAAACATTAA | |
| | | CCAAGGTCACCCAGTTATCAGAGGAGCAAACA | |
| | | GGGGCAAAGTCCACAGAGGGAGGCTGCTGGTA | |
| | | AACATTAACCAAGGTCACCCAGTTATCAGAGG | |
| | | AGCAAACAGGGGCAAAGTCCACAGAGGGAGG | |
| | | CTGCTGGTAAACATTAACCAAGGTCACCCAGTT | |
| | | ATCAGAGGAGCAAACAGGGGCAAAGTCCACAG | |
| | | AGGGAGGCTGCTGGTAAACATTAACCAAGGTC | |
| | | ACCCAGTTATCAGAGGAGCAAACAGGGGCAAA | |
| | | GTCCACAGAGGGAGGCTGCTGGTAAACATTAA | |
| | | CCAAGGTCACCCAGTTATCAGAGGAGCAAACA | |
| | | GGGGCAAAGTCCAT | |
| 10x_HNF4_ | 10× repeat of | GGGGGAGGCTGCTGGTAAACATTAACCAAGGT | 33 |
| FOXA_v1_ | HNF4_FOXA_v1 | CACCTCAGTTATCAGAGGAGCAAACAGGGACA | |
| Secondary_ | with poly-C/poly-G | AAGTCCACCGGGGGAGGCTGCTGGTAAACATT | |
| Struct_min_ | minimization v2 | AACCAAGGTCACCTCAGTTATCAGAGGAGCAA | |
| v2 | | ACAGGGACAAAGTCCACCGGGGGAGGCTGCTG | |
| | | GTAAACATTAACCAAGGTCACCTCAGTTATCAG | |
| | | AGGAGCAAACAGGGACAAAGTCCACCGGGGG | |
| | | AGGCTGCTGGTAAACATTAACCAAGGTCACCT | |
| | | CAGTTATCAGAGGAGCAAACAGGGACAAAGTC | |
| | | CACCGGGGGAGGCTGCTGGTAAACATTAACCA | |
| | | AGGTCACCTCAGTTATCAGAGGAGCAAACAGG | |
| | | GACAAAGTCCACCGGGGGAGGCTGCTGGTAAA | |
| | | CATTAACCAAGGTCACCTCAGTTATCAGAGGA | |
| | | GCAAACAGGGACAAAGTCCACCGGGGGAGGCT | |
| | | GCTGGTAAACATTAACCAAGGTCACCTCAGTTA | |
| | | TCAGAGGAGCAAACAGGGACAAAGTCCACCGG | |
| | | GGGAGGCTGCTGGTAAACATTAACCAAGGTCA | |
| | | CCTCAGTTATCAGAGGAGCAAACAGGGACAAA | |
| | | GTCCACCGGGGGAGGCTGCTGGTAAACATTAA | |
| | | CCAAGGTCACCTCAGTTATCAGAGGAGCAAAC | |
| | | AGGGACAAAGTCCACCGGGGGAGGCTGCTGGT | |
| | | AAACATTAACCAAGGTCACCTCAGTTATCAGA | |
| | | GGAGCAAACAGGGACAAAGTCCAC | |
| 10x_HNF4_ | 10× repeat of | AGGGGAGGCTGCTGGTAAACATTAACCAAGGT | 34 |
| FOXA_v1_ | HNF4_FOXA_v1 | CACCTCAGTTATCAGAGGAGCAAACAGGGACA | |
| Secondary_ | with poly-C/poly-G | AAGTCCACAGGGGGAGGCTGCTGGTAAACATT | |
| Struct_min_ | minimization and | AACCAAGGTCACCTCAGTTATCAGAGGAGCAA | |
| v2_CpG_min | CpG minimization v2 | ACAGGGACAAAGTCCACAGGGGGAGGCTGCTG | |
| | | GTAAACATTAACCAAGGTCACCTCAGTTATCAG | |
| | | AGGAGCAAACAGGGACAAAGTCCACAGGGGG | |
| | | AGGCTGCTGGTAAACATTAACCAAGGTCACCT | |
| | | CAGTTATCAGAGGAGCAAACAGGGACAAAGTC | |
| | | CACAGGGGGAGGCTGCTGGTAAACATTAACCA | |
| | | AGGTCACCTCAGTTATCAGAGGAGCAAACAGG | |
| | | GACAAAGTCCACAGGGGGAGGCTGCTGGTAAA | |
| | | CATTAACCAAGGTCACCTCAGTTATCAGAGGA | |
| | | GCAAACAGGGACAAAGTCCACAGGGGGAGGCT | |
| | | GCTGGTAAACATTAACCAAGGTCACCTCAGTTA | |
| | | TCAGAGGAGCAAACAGGGACAAAGTCCACAGG | |
| | | GGGAGGCTGCTGGTAAACATTAACCAAGGTCA | |
| | | CCTCAGTTATCAGAGGAGCAAACAGGGACAAA | |
| | | GTCCACAGGGGGAGGCTGCTGGTAAACATTAA | |
| | | CCAAGGTCACCTCAGTTATCAGAGGAGCAAAC | |
| | | AGGGACAAAGTCCACAGGGGGAGGCTGCTGGT | |
| | | AAACATTAACCAAGGTCACCTCAGTTATCAGA | |
| | | GGAGCAAACAGGGACAAAGTCCACA | |
| 10x_HNF4_ | 10× repeat of | GGGAGGCTGCTGGTAAACATTAACCAAGGTCA | 35 |
| FOXA_v1_ | HNF4_FOXA_v1 | CCCCAGTTATCAGAGGAGCAAACAAGGGCAAA | |
| Secondary_ | with poly-C/poly-G | GTCCACCGGGAGGCTGCTGGTAAACATTAACC | |
| Struct_min | minimization v3 | AAGGTCACCCCAGTTATCAGAGGAGCAAACAA | |
| _v3 | | GGGCAAAGTCCACCGGGAGGCTGCTGGTAAAC | |
| | | ATTAACCAAGGTCACCCCAGTTATCAGAGGAG | |
| | | CAAACAAGGGCAAAGTCCACCGGGAGGCTGCT | |
| | | GGTAAACATTAACCAAGGTCACCCCAGTTATC | |
| | | AGAGGAGCAAACAAGGGCAAAGTCCACCGGG | |
| | | AGGCTGCTGGTAAACATTAACCAAGGTCACCC | |
| | | CAGTTATCAGAGGAGCAAACAAGGGCAAAGTC | |
| | | CACCGGGAGGCTGCTGGTAAACATTAACCAAG | |
| | | GTCACCCCAGTTATCAGAGGAGCAAACAAGGG | |
| | | CAAAGTCCACCGGGAGGCTGCTGGTAAACATT | |
| | | AACCAAGGTCACCCCAGTTATCAGAGGAGCAA | |
| | | ACAAGGGCAAAGTCCACCGGGAGGCTGCTGGT | |
| | | AAACATTAACCAAGGTCACCCCAGTTATCAGA | |
| | | GGAGCAAACAAGGGCAAAGTCCACCGGGAGG | |
| | | CTGCTGGTAAACATTAACCAAGGTCACCCCAGT | |
| | | TATCAGAGGAGCAAACAAGGGCAAAGTCCACC | |
| | | GGGAGGCTGCTGGTAAACATTAACCAAGGTCA | |
| | | CCCCAGTTATCAGAGGAGCAAACAAGGGCAAA | |
| | | GTCCAC | |
| 10x_HNF4 | 10× repeat of | AGGGAGGCTGCTGGTAAACATTAACCAAGGTC | 36 |
| _FOXA_v1_ | HNF4_FOXA_v1 | ACCCCAGTTATCAGAGGAGCAAACAAGGGCAA | |
| Secondary_ | with poly-C/poly-G | AGTCCACAGGGAGGCTGCTGGTAAACATTAAC | |
| Struct_min_ | minimization and | CAAGGTCACCCCAGTTATCAGAGGAGCAAACA | |
| v3_CpG_min | CpG minimization v3 | AGGGCAAAGTCCACAGGGAGGCTGCTGGTAAA | |
| | | CATTAACCAAGGTCACCCCAGTTATCAGAGGA | |
| | | GCAAACAAGGGCAAAGTCCACAGGGAGGCTGC | |
| | | TGGTAAACATTAACCAAGGTCACCCCAGTTATC | |
| | | AGAGGAGCAAACAAGGGCAAAGTCCACAGGG | |
| | | AGGCTGCTGGTAAACATTAACCAAGGTCACCC | |
| | | CAGTTATCAGAGGAGCAAACAAGGGCAAAGTC | |
| | | CACAGGGAGGCTGCTGGTAAACATTAACCAAG | |
| | | GTCACCCCAGTTATCAGAGGAGCAAACAAGGG | |
| | | CAAAGTCCACAGGGAGGCTGCTGGTAAACATT | |
| | | AACCAAGGTCACCCCAGTTATCAGAGGAGCAA | |
| | | ACAAGGGCAAAGTCCACAGGGAGGCTGCTGGT | |
| | | AAACATTAACCAAGGTCACCCCAGTTATCAGA | |
| | | GGAGCAAACAAGGGCAAAGTCCACAGGGAGG | |
| | | CTGCTGGTAAACATTAACCAAGGTCACCCCAGT | |
| | | TATCAGAGGAGCAAACAAGGGCAAAGTCCACA | |
| | | GGGAGGCTGCTGGTAAACATTAACCAAGGTCA | |
| | | CCCCAGTTATCAGAGGAGCAAACAAGGGCAAA | |
| | | GTCCACA | |
| 10x_ | 10× repeat of the | GGGGGAGGCTGCTGGTGAATATTAACCAAGGT | 37 |
| hSerpEnh | human SERPINA1 | CACCCCAGTTATCGGAGGAGCAAACAGGGGCT | |
| | enhancer (“C” | AAGTCCACCGGGGGAGGCTGCTGGTGAATATT | |
| | spacer) | AACCAAGGTCACCCCAGTTATCGGAGGAGCAA | |
| | | ACAGGGGCTAAGTCCACCGGGGGAGGCTGCTG | |
| | | GTGAATATTAACCAAGGTCACCCCAGTTATCGG | |
| | | AGGAGCAAACAGGGGCTAAGTCCACCGGGGG | |
| | | AGGCTGCTGGTGAATATTAACCAAGGTCACCC | |
| | | CAGTTATCGGAGGAGCAAACAGGGGCTAAGTC | |
| | | CACCGGGGGAGGCTGCTGGTGAATATTAACCA | |
| | | AGGTCACCCCAGTTATCGGAGGAGCAAACAGG | |
| | | GGCTAAGTCCACCGGGGGAGGCTGCTGGTGAA | |
| | | TATTAACCAAGGTCACCCCAGTTATCGGAGGA | |
| | | GCAAACAGGGGCTAAGTCCACCGGGGGAGGCT | |
| | | GCTGGTGAATATTAACCAAGGTCACCCCAGTTA | |
| | | TCGGAGGAGCAAACAGGGGCTAAGTCCACCGG | |
| | | GGGAGGCTGCTGGTGAATATTAACCAAGGTCA | |
| | | CCCCAGTTATCGGAGGAGCAAACAGGGGCTAA | |
| | | GTCCACCGGGGGAGGCTGCTGGTGAATATTAA | |
| | | CCAAGGTCACCCCAGTTATCGGAGGAGCAAAC | |
| | | AGGGGCTAAGTCCACCGGGGGAGGCTGCTGGT | |
| | | GAATATTAACCAAGGTCACCCCAGTTATCGGA | |
| | | GGAGCAAACAGGGGCTAAGTCCAC | |
| 10x_ | 10× repeat of the | AGGGGAAGCTACTGGTGAATATTAACCAAGGT | 38 |
| Bushbaby_ | Bushbaby | CACCCAGTTATCAGGGAGCAAACAGGAGCTAA | |
| Aspacers | SERPINA1 enhancer | GTCCATAGGGGGAAGCTACTGGTGAATATTAA | |
| | with adenenine | CCAAGGTCACCCAGTTATCAGGGAGCAAACAG | |
| | nucleotide spacer | GAGCTAAGTCCATAGGGGGAAGCTACTGGTGA | |
| | | ATATTAACCAAGGTCACCCAGTTATCAGGGAG | |
| | | CAAACAGGAGCTAAGTCCATAGGGGGAAGCTA | |
| | | CTGGTGAATATTAACCAAGGTCACCCAGTTATC | |
| | | AGGGAGCAAACAGGAGCTAAGTCCATAGGGGG | |
| | | AAGCTACTGGTGAATATTAACCAAGGTCACCC | |
| | | AGTTATCAGGGAGCAAACAGGAGCTAAGTCCA | |
| | | TAGGGGGAAGCTACTGGTGAATATTAACCAAG | |
| | | GTCACCCAGTTATCAGGGAGCAAACAGGAGCT | |
| | | AAGTCCATAGGGGGAAGCTACTGGTGAATATT | |
| | | AACCAAGGTCACCCAGTTATCAGGGAGCAAAC | |
| | | AGGAGCTAAGTCCATAGGGGGAAGCTACTGGT | |
| | | GAATATTAACCAAGGTCACCCAGTTATCAGGG | |
| | | AGCAAACAGGAGCTAAGTCCATAGGGGGAAGC | |
| | | TACTGGTGAATATTAACCAAGGTCACCCAGTTA | |
| | | TCAGGGAGCAAACAGGAGCTAAGTCCATAGGG | |
| | | GGAAGCTACTGGTGAATATTAACCAAGGTCAC | |
| | | CCAGTTATCAGGGAGCAAACAGGAGCTAAGTC | |
| | | CAT | |
| Bushbaby_ | Bushbaby | GGGGGAAGCTACTGGTGAATATTAACCAAGGT | 39 |
| HN4F/FOX | SERPINA1 enhancer, | CACCCAGTTATCAGGGAGCAAACAGGAGCTAA | |
| v1_ | FOXA_HNF4_v1 | GTCCATAGGGGGAGGCTGCTGGTAAACATTAA | |
| HNF4mod | enhancer, HNF4 | CCAAGGTCACCCCAGTTATCAGAGGAGCAAAC | |
| | consensus binding | AGGGGCAAAGTCCACAGAGGGAGGCTGCTGGT | |
| | site enhancer | GAATATTAACCAAGGTCACCTCAGTTATCAGA | |
| | | GGAGCAAACAGGGGCAAAGTCCAT | |
| HNF4mod_ | HNF4 consensus | AGAGGGAGGCTGCTGGTGAATATTAACCAAGG | 40 |
| Bushbaby | binding site enhancer, | TCACCTCAGTTATCAGAGGAGCAAACAGGGGC | |
| Mod_HN4F/ | Bushbaby | AAAGTCCATAGAGGGAAGCTACTGGTGAATAT | |
| FOXv1 | SERPINA1 enhancer, | TAACCAAGGTCACCCAGTTATCAGGGAGCAAA | |
| | FOXA_HNF4_v1 | CAGGAGCTAAGTCCATAGGGGGAGGCTGCTGG | |
| | enhancer | TAAACATTAACCAAGGTCACCCCAGTTATCAG | |
| | | AGGAGCAAACAGGGGCAAAGTCCAC | |
| 3x_ | 3× repeat of | GGGGGAGGCTGCTGGTGAATATTAACCAAGGT | 41 |
| hSerpEnh_ | hSerpEnh with 2mer | CACCCCAGTTATCGGAGGAGCAAACAGGGGCT | |
| 2mer_ | spacers v1 (bold | AAGTCCACAAGGGGGAGGCTGCTGGTGAATAT | |
| spacers_v1 | underlined) | TAACCAAGGTCACCCCAGTTATCGGAGGAGCA | |
| | | AACAGGGGCTAAGTCCACCAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEn | 3× repeat of | GGGGGAGGCTGCTGGTGAATATTAACCAAGGT | 42 |
| h_2mer_ | hSerpEnh with 2mer | CACCCCAGTTATCGGAGGAGCAAACAGGGGCT | |
| spacers_ | spacers v2 (bold | AAGTCCACAAGGGGGAGGCTGCTGGTGAATAT | |
| v2 | underlined) | TAACCAAGGTCACCCCAGTTATCGGAGGAGCA | |
| | | AACAGGGGCTAAGTCCACCTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 43 |
| _2mer_ | hSerpEnh with 2mer | CACCCCAGTTATCGGAGGAGCAAACAGGGGCT | |
| spacers_ | spacers v3 (bold | AAGTCCACAAGGGGGAGGCTGCTGGTGAATAT | |
| v3 | underlined) | TAACCAAGGTCACCCCAGTTATCGGAGGAGCA | |
| | | AACAGGGGCTAAGTCCACTAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 44 |
| _2mer_ | hSerpEnh with 2mer | CACCCCAGTTATCGGAGGAGCAAACAGGGGCT | |
| spacers | spacers v4 (bold | AAGTCCACAAGGGGGAGGCTGCTGGTGAATAT | |
| _v4 | underlined) | TAACCAAGGTCACCCCAGTTATCGGAGGAGCA | |
| | | AACAGGGGCTAAGTCCACTTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 45 |
| _2mer_spa | hSerpEnh with 2mer | CACCCCAGTTATCGGAGGAGCAAACAGGGGCT | |
| cers_v5 | spacers v5 (bold | AAGTCCACCAGGGGGAGGCTGCTGGTGAATAT | |
| | underlined) | TAACCAAGGTCACCCCAGTTATCGGAGGAGCA | |
| | | AACAGGGGCTAAGTCCACAAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 46 |
| _2mer_ | hSerpEnh with 2mer | CACCCCAGTTATCGGAGGAGCAAACAGGGGCT | |
| spacers_ | spacers v6 (bold | AAGTCCACCAGGGGGAGGCTGCTGGTGAATAT | |
| v6 | underlined) | TAACCAAGGTCACCCCAGTTATCGGAGGAGCA | |
| | | AACAGGGGCTAAGTCCACCTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 47 |
| _2mer_ | hSerpEnh with 2mer | CACCCCAGTTATCGGAGGAGCAAACAGGGGCT | |
| spacers_v7 | spacers v7 (bold | AAGTCCACCAGGGGGAGGCTGCTGGTGAATAT | |
| | underlined) | TAACCAAGGTCACCCCAGTTATCGGAGGAGCA | |
| | | AACAGGGGCTAAGTCCACTAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 48 |
| _2mer_ | hSerpEnh with 2mer | CACCCCAGTTATCGGAGGAGCAAACAGGGGCT | |
| spacers_v8 | spacers v8 (bold | AAGTCCACCAGGGGGAGGCTGCTGGTGAATAT | |
| | underlined) | TAACCAAGGTCACCCCAGTTATCGGAGGAGCA | |
| | | AACAGGGGCTAAGTCCACTTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 49 |
| _2mer_ | hSerpEnh with 2mer | CACCCCAGTTATCGGAGGAGCAAACAGGGGCT | |
| spacers_v9 | spacers v9 (bold | AAGTCCACCTGGGGGAGGCTGCTGGTGAATAT | |
| | underlined) | TAACCAAGGTCACCCCAGTTATCGGAGGAGCA | |
| | | AACAGGGGCTAAGTCCACAAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 50 |
| _2mer_ | hSerpEnh with 2mer | CACCCCAGTTATCGGAGGAGCAAACAGGGGCT | |
| spacers_v10 | spacers v10 (bold | AAGTCCACCTGGGGGAGGCTGCTGGTGAATAT | |
| | underlined) | TAACCAAGGTCACCCCAGTTATCGGAGGAGCA | |
| | | AACAGGGGCTAAGTCCACCAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 51 |
| _2mer_ | hSerpEnh with 2mer | CACCCCAGTTATCGGAGGAGCAAACAGGGGCT | |
| spacers_v11 | spacers v11 (bold | AAGTCCACCTGGGGGAGGCTGCTGGTGAATAT | |
| | underlined) | TAACCAAGGTCACCCCAGTTATCGGAGGAGCA | |
| | | AACAGGGGCTAAGTCCACTAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 52 |
| _2mer_ | hSerpEnh with 2mer | CACCCCAGTTATCGGAGGAGCAAACAGGGGCT | |
| spacers_v12 | spacers v12 (bold | AAGTCCACCTGGGGGAGGCTGCTGGTGAATAT | |
| | underlined) | TAACCAAGGTCACCCCAGTTATCGGAGGAGCA | |
| | | AACAGGGGCTAAGTCCACTTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 53 |
| _2mer_ | hSerpEnh with 2mer | CACCCCAGTTATCGGAGGAGCAAACAGGGGCT | |
| spacers_v13 | spacers v13 (bold | AAGTCCACTAGGGGGAGGCTGCTGGTGAATAT | |
| | underlined) | TAACCAAGGTCACCCCAGTTATCGGAGGAGCA | |
| | | AACAGGGGCTAAGTCCACAAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 54 |
| _2mer_ | hSerpEnh with 2mer | CACCCCAGTTATCGGAGGAGCAAACAGGGGCT | |
| spacers_v14 | spacers v14 (bold | AAGTCCACTAGGGGGAGGCTGCTGGTGAATAT | |
| | underlined) | TAACCAAGGTCACCCCAGTTATCGGAGGAGCA | |
| | | AACAGGGGCTAAGTCCACCAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 55 |
| _2mer_ | hSerpEnh with 2mer | CACCCCAGTTATCGGAGGAGCAAACAGGGGCT | |
| spacers_v15 | spacers v15 (bold | AAGTCCACTAGGGGGAGGCTGCTGGTGAATAT | |
| | underlined) | TAACCAAGGTCACCCCAGTTATCGGAGGAGCA | |
| | | AACAGGGGCTAAGTCCACCTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 56 |
| _2mer_ | hSerpEnh with 2mer | CACCCCAGTTATCGGAGGAGCAAACAGGGGCT | |
| spacers_v16 | spacers v16 (bold | AAGTCCACTAGGGGGAGGCTGCTGGTGAATAT | |
| | underlined) | TAACCAAGGTCACCCCAGTTATCGGAGGAGCA | |
| | | AACAGGGGCTAAGTCCACTTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 57 |
| _2mer_ | hSerpEnh with 2mer | CACCCCAGTTATCGGAGGAGCAAACAGGGGCT | |
| spacers_v17 | spacers v17 (bold | AAGTCCACTTGGGGGAGGCTGCTGGTGAATAT | |
| | underlined) | TAACCAAGGTCACCCCAGTTATCGGAGGAGCA | |
| | | AACAGGGGCTAAGTCCACAAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 58 |
| _2mer_spacers | hSerpEnh with 2mer | CACCCCAGTTATCGGAGGAGCAAACAGGGGCT | |
| _v18 | spacers v18 (bold | AAGTCCACTTGGGGGAGGCTGCTGGTGAATAT | |
| | underlined) | TAACCAAGGTCACCCCAGTTATCGGAGGAGCA | |
| | | AACAGGGGCTAAGTCCACCAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 59 |
| _2mer_ | hSerpEnh with 2mer | CACCCCAGTTATCGGAGGAGCAAACAGGGGCT | |
| spacers_v19 | spacers v19 (bold | AAGTCCACTTGGGGGAGGCTGCTGGTGAATAT | |
| | underlined) | TAACCAAGGTCACCCCAGTTATCGGAGGAGCA | |
| | | AACAGGGGCTAAGTCCACCTGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 60 |
| _2mer_ | hSerpEnh with 2mer | CACCCCAGTTATCGGAGGAGCAAACAGGGGCT | |
| spacers_v20 | spacers v20 (bold | AAGTCCACTTGGGGGAGGCTGCTGGTGAATAT | |
| | underlined) | TAACCAAGGTCACCCCAGTTATCGGAGGAGCA | |
| | | AACAGGGGCTAAGTCCACTAGGGGGAGGCTGC | |
| | | TGGTGAATATTAACCAAGGTCACCCCAGTTATC | |
| | | GGAGGAGCAAACAGGGGCTAAGTCCAC | |
| 3x_hSerpEnh | 3× repeat of | GGGGGAGGCTGCTGGTGAATATTAACCAAGGT | 61 |
| _3mer_ | hSerpEnh with 3mer | CACCCCAGTTATCGGAGGAGCAAACAGGGGCT | |
| spacers_v1 | spacers v1 (bold | AAGTCCACTTAGGGGGAGGCTGCTGGTGAATA | |
| | underlined) | TTAACCAAGGTCACCCCAGTTATCGGAGGAGC | |
| | | AAACAGGGGCTAAGTCCACTGTGGGGGAGGCT | |
| | | GCTGGTGAATATTAACCAAGGTCACCCCAGTTA | |
| | | TCGGAGGAGCAAACAGGGGCTAAGTCCAC | |
| 3x_hSerpEnh | 3× repeat of | GGGGGAGGCTGCTGGTGAATATTAACCAAGGT | 62 |
| _3mer_ | hSerpEnh with 3mer | CACCCCAGTTATCGGAGGAGCAAACAGGGGCT | |
| spacers_v2 | spacers v2 (bold | AAGTCCACAGAGGGGGAGGCTGCTGGTGAATA | |
| | underlined) | TTAACCAAGGTCACCCCAGTTATCGGAGGAGC | |
| | | AAACAGGGGCTAAGTCCACTGAGGGGGAGGCT | |
| | | GCTGGTGAATATTAACCAAGGTCACCCCAGTTA | |
| | | TCGGAGGAGCAAACAGGGGCTAAGTCCAC | |
| 3x_hSerpEnh | 3× repeat of | GGGGGAGGCTGCTGGTGAATATTAACCAAGGT | 63 |
| _3mer_ | hSerpEnh with 3mer | CACCCCAGTTATCGGAGGAGCAAACAGGGGCT | |
| spacers_v3 | spacers v3 (bold | AAGTCCACACTGGGGGAGGCTGCTGGTGAATA | |
| | underlined) | TTAACCAAGGTCACCCCAGTTATCGGAGGAGC | |
| | | AAACAGGGGCTAAGTCCACCAAGGGGGAGGCT | |
| | | GCTGGTGAATATTAACCAAGGTCACCCCAGTTA | |
| | | TCGGAGGAGCAAACAGGGGCTAAGTCCAC | |
| 3x_hSerpEnh | 3× repeat of | GGGGGAGGCTGCTGGTGAATATTAACCAAGGT | 64 |
| _5mer_ | hSerpEnh with 5mer | CACCCCAGTTATCGGAGGAGCAAACAGGGGCT | |
| spacers_v1 | spacers v1 (bold | AAGTCCACACATAGGGGGAGGCTGCTGGTGAA | |
| | underlined) | TATTAACCAAGGTCACCCCAGTTATCGGAGGA | |
| | | GCAAACAGGGGCTAAGTCCACCTGTAGGGGGA | |
| | | GGCTGCTGGTGAATATTAACCAAGGTCACCCC | |
| | | AGTTATCGGAGGAGCAAACAGGGGCTAAGTCC | |
| | | AC | |
| 3x_hSerpEnh | 3× repeat of | GGGGGAGGCTGCTGGTGAATATTAACCAAGGT | |
| _5mer_ | hSerpEnh with 5mer | CACCCCAGTTATCGGAGGAGCAAACAGGGGCT | |
| spacers_v2 | spacers v2 (bold | AAGTCCACAACAAGGGGGAGGCTGCTGGTGAA | |
| | underlined) | TATTAACCAAGGTCACCCCAGTTATCGGAGGA | |
| | | GCAAACAGGGGCTAAGTCCACCATCAGGGGGA | |
| | | GGCTGCTGGTGAATATTAACCAAGGTCACCCC | 65 |
| | | AGTTATCGGAGGAGCAAACAGGGGCTAAGTCC | |
| | | AC | |
| 3x_hSerpEnh | 3× repeat of | GGGGGAGGCTGCTGGTGAATATTAACCAAGGT | 66 |
| _5mer_ | hSerpEnh with 5mer | CACCCCAGTTATCGGAGGAGCAAACAGGGGCT | |
| spacers_v3 | spacers v3 (bold | AAGTCCACCAATTGGGGGAGGCTGCTGGTGAA | |
| | underlined) | TATTAACCAAGGTCACCCCAGTTATCGGAGGA | |
| | | GCAAACAGGGGCTAAGTCCACTTGCTGGGGGA | |
| | | GGCTGCTGGTGAATATTAACCAAGGTCACCCC | |
| | | AGTTATCGGAGGAGCAAACAGGGGCTAAGTCC | |
| | | AC | |
| 3x_hSerpEnh | 3× repeat of | GGGGGAGGCTGCTGGTGAATATTAACCAAGGT | 67 |
| _11mer_ | hSerpEnh with 11mer | CACCCCAGTTATCGGAGGAGCAAACAGGGGCT | |
| spacers_v1 | spacers v1 (bold | AAGTCCACCCTTGGGACCAGGGGGAGGCTGC | |
| | underlined) | TGGTGAATATTAACCAAGGTCACCCCAGTTATC | |
| | | GGAGGAGCAAACAGGGGCTAAGTCCACAAGC | |
| | | TGTTCCAGGGGGAGGCTGCTGGTGAATATTAA | |
| | | CCAAGGTCACCCCAGTTATCGGAGGAGCAAAC | |
| | | AGGGGCTAAGTCCAC | |
| 3x_hSerpEnh | 3× repeat of | GGGGGAGGCTGCTGGTGAATATTAACCAAGGT | 68 |
| _11mer_ | hSerpEnh with 11mer | CACCCCAGTTATCGGAGGAGCAAACAGGGGCT | |
| spacers_v2 | spacers v2 (bold | AAGTCCACAGGCTGGTTGAGGGGGAGGCTGC | |
| | underlined) | TGGTGAATATTAACCAAGGTCACCCCAGTTATC | |
| | | GGAGGAGCAAACAGGGGCTAAGTCCACTGATA | |
| | | ATAGCTGGGGGAGGCTGCTGGTGAATATTAAC | |
| | | CAAGGTCACCCCAGTTATCGGAGGAGCAAACA | |
| | | GGGGCTAAGTCCAC | |
| 3x_hSerpEnh | 3× repeat of | GGGGGAGGCTGCTGGTGAATATTAACCAAGGT | 69 |
| _11mer_ | hSerpEnh with 11mer | CACCCCAGTTATCGGAGGAGCAAACAGGGGCT | |
| spacers_v3 | spacers v3 (bold | AAGTCCACCATTCTGCTTTGGGGGAGGCTGCT | |
| | underlined) | GGTGAATATTAACCAAGGTCACCCCAGTTATCG | |
| | | GAGGAGCAAACAGGGGCTAAGTCCACTTGATT | |
| | | AAGAAGGGGGAGGCTGCTGGTGAATATTAACC | |
| | | AAGGTCACCCCAGTTATCGGAGGAGCAAACAG | |
| | | GGGCTAAGTCCAC | |
| 3x_hSerpEnh | 3× repeat of | GGGGGAGGCTGCTGGTGAATATTAACCAAGGT | 70 |
| _11mer_ | hSerpEnh with 11mer | CACCCCAGTTATCGGAGGAGCAAACAGGGGCT | |
| spacers_HNF | spacers (bold | AAGTCCACAACAAAGTCCAGGGGGAGGCTGCT | |
| 4former_ | underlined) with | GGTGAATATTAACCAAGGTCACCCCAGTTATCG | |
| spacers_FOX | HNF4 binding site in | GAGGAGCAAACAGGGGCTAAGTCCACCTTGTA | |
| Afor | orientation 1 & | AACAAGGGGGAGGCTGCTGGTGAATATTAACC | |
| | FOXA binding site in | AAGGTCACCCCAGTTATCGGAGGAGCAAACAG | |
| | orientation 1 | GGGCTAAGTCCAC | |
| 3x_hSerpEnh | 3× repeat of | GGGGGAGGCTGCTGGTGAATATTAACCAAGGT | 71 |
| _11mer_ | hSerpEnh with 11mer | CACCCCAGTTATCGGAGGAGCAAACAGGGGCT | |
| spacers_HNF | spacers (bold | AAGTCCACTGCAAAGTCCTGGGGGAGGCTGCT | |
| 4former_ | underlined) with | GGTGAATATTAACCAAGGTCACCCCAGTTATCG | |
| spacers_FOX | HNF4 binding site in | GAGGAGCAAACAGGGGCTAAGTCCACAGTGTT | |
| Arev | orientation 1 & | TACAAGGGGGAGGCTGCTGGTGAATATTAACC | |
| | FOXA binding site in | AAGGTCACCCCAGTTATCGGAGGAGCAAACAG | |
| | orientation 2 | GGGCTAAGTCCAC | |
| 3x_hSerpEnh | 3× repeat of | GGGGGAGGCTGCTGGTGAATATTAACCAAGGT | 72 |
| _11mer_ | hSerpEnh with 11mer | CACCCCAGTTATCGGAGGAGCAAACAGGGGCT | |
| spacers_HNF | spacers (bold | AAGTCCACAGGACTTTGAAGGGGGAGGCTGCT | |
| 4revmer_ | underlined) with | GGTGAATATTAACCAAGGTCACCCCAGTTATCG | |
| spacers_FOX | HNF4 binding site in | GAGGAGCAAACAGGGGCTAAGTCCACAGTGT | |
| Afor | orientation 2 & | AAACAAGGGGGAGGCTGCTGGTGAATATTAAC | |
| | FOXA binding site in | CAAGGTCACCCCAGTTATCGGAGGAGCAAACA | |
| | orientation 1 | GGGGCTAAGTCCAC | |
| 3x_hSerpEnh | 3× repeat of | GGGGGAGGCTGCTGGTGAATATTAACCAAGGT | 73 |
| _11mer_ | hSerpEnh with 11mer | CACCCCAGTTATCGGAGGAGCAAACAGGGGCT | |
| spacers_HNF | spacers (bold | AAGTCCACTGGACTTTGGTGGGGGAGGCTGCT | |
| 4revmer_ | underlined) with | GGTGAATATTAACCAAGGTCACCCCAGTTATCG | |
| spacers_FOX | HNF4 binding site in | GAGGAGCAAACAGGGGCTAAGTCCACTCTGTT | |
| Arev | orientation 2 & | TACAAGGGGGAGGCTGCTGGTGAATATTAACC | |
| | FOXA binding site in | AAGGTCACCCCAGTTATCGGAGGAGCAAACAG | |
| | orientation 2 | GGGCTAAGTCCAC | |
| 3x_hSerpEnh | 3× repeat of | GGGGGAGGCTGCTGGTGAATATTAACCAAGGT | 74 |
| _30mer_ | hSerpEnh with 30mer | CACCCCAGTTATCGGAGGAGCAAACAGGGGCT | |
| spacers_v1 | spacers v1 (bold | AAGTCCACCTGCTTGACATCTGCAGTAATCT | |
| | underlined) | TTGATTAGGGGGAGGCTGCTGGTGAATATTAA | |
| | | CCAAGGTCACCCCAGTTATCGGAGGAGCAAAC | |
| | | AGGGGCTAAGTCCACCTCTGATACTTTGATAT | |
| | | CTAGTCTACTGCTGGGGGAGGCTGCTGGTGAA | |
| | | TATTAACCAAGGTCACCCCAGTTATCGGAGGA | |
| | | GCAAACAGGGGCTAAGTCCAC | |
| 3x_hSerpEnh | 3× repeat of | GGGGGAGGCTGCTGGTGAATATTAACCAAGGT | 75 |
| _30mer_ | hSerpEnh with 30mer | CACCCCAGTTATCGGAGGAGCAAACAGGGGCT | |
| spacers_v2 | spacers v2 (bold | AAGTCCACCACTTGTATTTAATCATAACTACT | |
| | underlined) | TAGCAAGGGGGAGGCTGCTGGTGAATATTAAC | |
| | | CAAGGTCACCCCAGTTATCGGAGGAGCAAACA | |
| | | GGGGCTAAGTCCACTAACATCTTACAAACTAA | |
| | | AGTTAGATAGTAGGGGGAGGCTGCTGGTGAAT | |
| | | ATTAACCAAGGTCACCCCAGTTATCGGAGGAG | |
| | | CAAACAGGGGCTAAGTCCAC | |
| 3x_hSerpEnh | 3× repeat of | GGGGGAGGCTGCTGGTGAATATTAACCAAGGT | 76 |
| _30mer_ | hSerpEnh with 30mer | CACCCCAGTTATCGGAGGAGCAAACAGGGGCT | |
| spacers_v3 | spacers v3 (bold | AAGTCCACATAGAAGAATTTCTTACATTGTGT | |
| | underlined) | GAACCTGGGGGAGGCTGCTGGTGAATATTAAC | |
| | | CAAGGTCACCCCAGTTATCGGAGGAGCAAACA | |
| | | GGGGCTAAGTCCACATTGAAGTGCAAAGTCA | |
| | | CTAATATTAAGCAGGGGGAGGCTGCTGGTGAA | |
| | | TATTAACCAAGGTCACCCCAGTTATCGGAGGA | |
| | | GCAAACAGGGGCTAAGTCCAC | |
| 3x_hSerpEnh | 3× repeat of | GGGGGAGGCTGCTGGTGAATATTAACCAAGGT | 77 |
| _30mer_ | hSerpEnh with 30mer | CACCCCAGTTATCGGAGGAGCAAACAGGGGCT | |
| spacers_HNF | spacers (bold | AAGTCCACATAATTAAAGTCAAAGTCCTCAC | |
| 4former_ | underlined) with | TGCTAGTGGGGGAGGCTGCTGGTGAATATTAA | |
| spacers_FOX | HNF4 binding site in | CCAAGGTCACCCCAGTTATCGGAGGAGCAAAC | |
| Afor | orientation 1 & | AGGGGCTAAGTCCACACAATTAGAGCTGTAA | |
| | FOXA binding site in | ACATAATTTGTGTAGGGGGAGGCTGCTGGTGA | |
| | orientation 1 | ATATTAACCAAGGTCACCCCAGTTATCGGAGG | |
| | | AGCAAACAGGGGCTAAGTCCAC | |
| 3x_hSerpEnh | 3× repeat of | GGGGGAGGCTGCTGGTGAATATTAACCAAGGT | 78 |
| _30mer_ | hSerpEnh with 30mer | CACCCCAGTTATCGGAGGAGCAAACAGGGGCT | |
| spacers_HNF | spacers (bold | AAGTCCACTTATTTGCACTCAAAGTCCACTTT | |
| 4former_ | underlined) with | ATTACAGGGGGAGGCTGCTGGTGAATATTAAC | |
| spacers_FOX | HNF4 binding site in | CAAGGTCACCCCAGTTATCGGAGGAGCAAACA | |
| Arev | orientation 1 & | GGGGCTAAGTCCACTCAATCATAAGTGTTTAC | |
| | FOXA binding site in | AAGTTTAAGATTGGGGGAGGCTGCTGGTGAAT | |
| | orientation 2 | ATTAACCAAGGTCACCCCAGTTATCGGAGGAG | |
| | | CAAACAGGGGCTAAGTCCAC | |
| 3x_hSerpEnh | 3× repeat of | GGGGGAGGCTGCTGGTGAATATTAACCAAGGT | 79 |
| _30mer_ | hSerpEnh with 30mer | CACCCCAGTTATCGGAGGAGCAAACAGGGGCT | |
| spacers_HNF | spacers (bold | AAGTCCACAGTTGCTGTGTGGACTTTGTCAC | |
| 4revmer_ | underlined) with | TGCAAGAGGGGGAGGCTGCTGGTGAATATTAA | |
| spacers_FOX | HNF4 binding site in | CCAAGGTCACCCCAGTTATCGGAGGAGCAAAC | |
| Afor | orientation 2 & | AGGGGCTAAGTCCACAACAGCATATTTGTAAA | |
| | FOXA binding site in | CAGTTCTATTAGTGGGGGAGGCTGCTGGTGAA | |
| | orientation 1 | TATTAACCAAGGTCACCCCAGTTATCGGAGGA | |
| | | GCAAACAGGGGCTAAGTCCAC | |
| 3x_hSerpEnh | 3× repeat of | GGGGGAGGCTGCTGGTGAATATTAACCAAGGT | 80 |
| _30mer_ | hSerpEnh with 30mer | CACCCCAGTTATCGGAGGAGCAAACAGGGGCT | |
| spacers_HNF | spacers (bold | AAGTCCACATTAACTATTGGGACTTTGGTTA | |
| 4revmer_ | underlined) with | ACAACAAGGGGGAGGCTGCTGGTGAATATTAA | |
| spacers_FOX | HNF4 binding site in | CCAAGGTCACCCCAGTTATCGGAGGAGCAAAC | |
| Arev | orientation 2 & | AGGGGCTAAGTCCACCAGAGACTTATTGTTTA | |
| | FOXA binding site in | CAGCTAACTATCTGGGGGAGGCTGCTGGTGAA | |
| | orientation 2 | TATTAACCAAGGTCACCCCAGTTATCGGAGGA | |
| | | GCAAACAGGGGCTAAGTCCAC | |
| 3x_Tibetan | 3 repeats of | GGGGGAGGCTGCTGGTAAACATTAACCAAGGT | 138 |
| antelope_ | SERPINA1 enhancer | CACCCCAGTTATCAGAGGAACAAACAAGGACT | |
| SERPINA1 | from tibetan | AAGTCCATTGGGGGAGGCTGCTGGTAAACATT | |
| enhancer | antelope, | AACCAAGGTCACCCCAGTTATCAGAGGAACAA | |
| | separated by T. | ACAAGGACTAAGTCCATTGGGGGAGGCTGCTG | |
| | | GTAAACATTAACCAAGGTCACCCCAGTTATCA | |
| | | GAGGAACAAACAAGGACTAAGTCCAT | |
| 3x_Armadil | 3 repeats of | GGGGGAGGCTGCTAGTGAACATTAACCAAGGT | 139 |
| 1o_CpGmin_ | SERPINA1 enhancer | CACCCAGTTATCAGAGGAGCAAACAGGGACTA | |
| SERPINA1_ | from armadillo with | AGTCCACTGGGGGAGGCTGCTAGTGAACATTA | |
| enhancer | CpG removed, | ACCAAGGTCACCCAGTTATCAGAGGAGCAAAC | |
| | separated by T. | AGGGACTAAGTCCACTGGGGGAGGCTGCTAGT | |
| | | GAACATTAACCAAGGTCACCCAGTTATCAGAG | |
| | | GAGCAAACAGGGACTAAGTCCAC | |

In some embodiments, the expression cassette comprises a regulatory element (e.g., an enhancer) comprising 3× repeat of the human SERPINA1 enhancer with FOXA & HNF4 consensus sites. In certain embodiment, the regulatory element comprising the 3× repeat of the human SERPINA1 enhancer with FOXA & HNF4 consensus sites comprises SEQ ID NO: 1.

In some embodiments, the expression cassette comprises a regulatory element (e.g., an enhancer) comprising 3× repeat of HNF4_FOXA_v1 with CpG minimization. In certain embodiment, the regulatory element comprising the 3× repeat of HNF4_FOXA_v1 with CpG minimization comprises SEQ ID NO: 2.

In some embodiments, the expression cassette comprises a regulatory element (e.g., an enhancer) comprising 3× repeat of HNF4_FOXA_v1 with poly-C/poly-G minimization v1. In certain embodiment, the regulatory element comprising the 3× repeat of HNF4_FOXA_v1 with poly-C/poly-G minimization v1 comprises SEQ ID NO: 3.

In some embodiments, the expression cassette comprises a regulatory element (e.g., an enhancer) comprising 3× repeat of HNF4_FOXA_v1 with poly-C/poly-G minimization and CpG minimization v1. In certain embodiment, the regulatory element comprising the 3× repeat of HNF4_FOXA_v1 with poly-C/poly-G minimization and CpG minimization v1 comprises SEQ ID NO: 4.

In some embodiments, the expression cassette comprises a regulatory element (e.g., an enhancer) comprising 3× repeat of HNF4_FOXA_v1 with poly-C/poly-G minimization v2. In certain embodiment, the regulatory element comprising the 3× repeat of HNF4_FOXA_v1 with poly-C/poly-G minimization v2 comprises SEQ ID NO: 5.

In some embodiments, the expression cassette comprises a regulatory element (e.g., an enhancer) comprising 3× repeat of HNF4_FOXA_v1 with poly-C/poly-G minimization and CpG minimization v2. In certain embodiment, the regulatory element comprising the 3× repeat of HNF4_FOXA_v1 with poly-C/poly-G minimization and CpG minimization v2 comprises SEQ ID NO: 6.

In some embodiments, the expression cassette comprises a regulatory element (e.g., an enhancer) comprising 3× repeat of HNF4_FOXA_v1 with poly-C/poly-G minimization v3. In certain embodiment, the regulatory element comprising the 3× repeat of HNF4_FOXA_v1 with poly-C/poly-G minimization v3 comprises SEQ ID NO: 7.

In some embodiments, the expression cassette comprises a regulatory element (e.g., an enhancer) comprising 3× repeat of HNF4_FOXA_v1 with poly-C/poly-G minimization and CpG minimization v3. In certain embodiment, the regulatory element comprising the 3× repeat of HNF4_FOXA_v1 with poly-C/poly-G minimization and CpG minimization v3 comprises SEQ ID NO: 8.

In some embodiments, the expression cassette comprises a regulatory element (e.g., an enhancer) comprising 3× repeat of HNF4_FOXA_v1 with poly-C/poly-G minimization v4. In certain embodiment, the regulatory element comprising the 3× repeat of HNF4_FOXA_v1 with poly-C/poly-G minimization v4 comprises SEQ ID NO: 9.

In some embodiments, the expression cassette comprises a regulatory element (e.g., an enhancer) comprising 3× repeat of HNF4_FOXA_v1 with poly-C/poly-G minimization v5. In certain embodiment, the regulatory element comprising the 3× repeat of HNF4_FOXA_v1 with poly-C/poly-G minimization v5 comprises SEQ ID NO: 10.

In some embodiments, the expression cassette comprises a regulatory element (e.g., an enhancer) comprising 3× repeat of HNF4_FOXA_v1 with poly-C/poly-G minimization v6. In certain embodiment, the regulatory element comprising the 3× repeat of HNF4_FOXA_v1 with poly-C/poly-G minimization v6 comprises SEQ ID NO: 11.

In some embodiments, the expression cassette comprises a regulatory element (e.g., an enhancer) comprising 3× repeat of the Chinese Tree Shrew SERPINA1 enhancer. In certain embodiment, the regulatory element comprising the 3× repeat of the Chinese Tree Shrew SERPINA1 enhancer comprises SEQ ID NO: 12.

In some embodiments, the expression cassette comprises a regulatory element (e.g., an enhancer) comprising 3× repeat of the Chinese Tree Shrew SERPINA1 enhancer with CpG minimization. In certain embodiment, the regulatory element comprising the 3× repeat of the Chinese Tree Shrew SERPINA1 enhancer with CpG minimization comprises SEQ ID NO: 13.

In some embodiments, the expression cassette comprises a regulatory element (e.g., an enhancer) comprising 3× repeat of the human SERPINA1 enhancer with 1 adenine between repeats. In certain embodiment, the regulatory element comprising the 3× repeat of the human SERPINA1 enhancer with 1 adenine between repeats comprises SEQ ID NO: 14.

In some embodiments, the expression cassette comprises a regulatory element (e.g., an enhancer) comprising 3× repeat of the Bushbaby SERPINA1 enhancer with adenine nucleotide spacer. In certain embodiment, the regulatory element comprising the 3× repeat of the Bushbaby SERPINA1 enhancer with adenine nucleotide spacer comprises SEQ ID NO: 15.

In some embodiments, the expression cassette comprises a regulatory element (e.g., an enhancer) comprising 5× repeat of HNF4_FOXA_v1. In certain embodiment, the regulatory element comprising the 5× repeat of HNF4_FOXA_v1 comprises SEQ ID NO: 16.

In some embodiments, the expression cassette comprises a regulatory element (e.g., an enhancer) comprising 5× repeat of HNF4_FOXA_v1 with poly-C/poly-G minimization v1. In certain embodiment, the regulatory element comprising the 5× repeat of HNF4_FOXA_v1 with poly-C/poly-G minimization v1 comprises SEQ ID NO: 17.

In some embodiments, the expression cassette comprises a regulatory element (e.g., an enhancer) comprising 5× repeat of HNF4_FOXA_v1 with poly-C/poly-G minimization and CpG minimization v1. In certain embodiment, the regulatory element comprising the 5× repeat of HNF4_FOXA_v1 with poly-C/poly-G minimization and CpG minimization v1 comprises SEQ ID NO: 18.

In some embodiments, the expression cassette comprises a regulatory element (e.g., an enhancer) comprising 5× repeat of HNF4_FOXA_v1 with poly-C/poly-G minimization v2. In certain embodiment, the regulatory element comprising the 5× repeat of HNF4_FOXA_v1 with poly-C/poly-G minimization v2 comprises SEQ ID NO: 19.

In some embodiments, the expression cassette comprises a regulatory element (e.g., an enhancer) comprising 5× repeat of HNF4_FOXA_v1 with poly-C/poly-G minimization and CpG minimization v2. In certain embodiment, the regulatory element comprising the 5× repeat of HNF4_FOXA_v1 with poly-C/poly-G minimization and CpG minimization v2 comprises SEQ ID NO: 20.

In some embodiments, the expression cassette comprises a regulatory element (e.g., an enhancer) comprising 5× repeat of HNF4_FOXA_v1 with poly-C/poly-G minimization v3. In certain embodiment, the regulatory element comprising the 5× repeat of HNF4_FOXA_v1 with poly-C/poly-G minimization v3 comprises SEQ ID NO: 21.

In some embodiments, the expression cassette comprises a regulatory element (e.g., an enhancer) comprising 5× repeat of HNF4_FOXA_v1 with poly-C/poly-G minimization and CpG minimization v3. In certain embodiment, the regulatory element comprising the 5× repeat of HNF4_FOXA_v1 with poly-C/poly-G minimization and CpG minimization v3 comprises SEQ ID NO: 22.

In some embodiments, the expression cassette comprises a regulatory element (e.g., an enhancer) comprising 5× repeat of HNF4_FOXA_v1 with poly-C/poly-G minimization v4. In certain embodiment, the regulatory element comprising the 5× repeat of HNF4_FOXA_v1 with poly-C/poly-G minimization v4 comprises SEQ ID NO: 23.

In some embodiments, the expression cassette comprises a regulatory element (e.g., an enhancer) comprising 5× repeat of HNF4_FOXA_v1 with poly-C/poly-G minimization v5. In certain embodiment, the regulatory element comprising the 5× repeat of HNF4_FOXA_v1 with poly-C/poly-G minimization v5 comprises SEQ ID NO: 24.

In some embodiments, the expression cassette comprises a regulatory element (e.g., an enhancer) comprising 5× repeat of HNF4_FOXA_v1 with poly-C/poly-G minimization v6. In certain embodiment, the regulatory element comprising the 5× repeat of HNF4_FOXA_v1 with poly-C/poly-G minimization v6 comprises SEQ ID NO: 25.

In some embodiments, the expression cassette comprises a regulatory element (e.g., an enhancer) comprising 5× repeat of the Chinese Tree Shrew SERPINA1 enhancer. In certain embodiment, the regulatory element comprising the 5× repeat of the Chinese Tree Shrew SERPINA1 enhancer comprises SEQ ID NO: 26.

In some embodiments, the expression cassette comprises a regulatory element (e.g., an enhancer) comprising 5× repeat of the Chinese Tree Shrew SERPINA1 enhancer with CpG minimization. In certain embodiment, the regulatory element comprising the 5× repeat of the Chinese Tree Shrew SERPINA1 enhancer with CpG minimization comprises SEQ ID NO: 27.

In some embodiments, the expression cassette comprises a regulatory element (e.g., an enhancer) comprising 5× repeat of the Bushbaby SERPINA1 enhancer with adenenine nucleotide spacer. In certain embodiment, the regulatory element comprising the 5× repeat of the Bushbaby SERPINA1 enhancer with adenenine nucleotide spacer comprises SEQ ID NO: 28.

In some embodiments, the expression cassette comprises a regulatory element (e.g., an enhancer) comprising 5× repeat of the human SERPINA1 enhancer. In certain embodiment, the regulatory element comprising the 5× repeat of the human SERPINA1 enhancer comprises SEQ ID NO: 29.

In some embodiments, the expression cassette comprises a regulatory element (e.g., an enhancer) comprising 10× repeat of HNF4_FOXA_v1. In certain embodiment, the regulatory element comprising the 10× repeat of HNF4_FOXA_v1 comprises SEQ ID NO: 30.

In some embodiments, the expression cassette comprises a regulatory element (e.g., an enhancer) comprising 10× repeat of HNF4_FOXA_v1 with poly-C/poly-G minimization v1. In certain embodiment, the regulatory element comprising the 10× repeat of HNF4_FOXA_v1 with poly-C/poly-G minimization v1 comprises SEQ ID NO: 31.

In some embodiments, the expression cassette comprises a regulatory element (e.g., an enhancer) comprising 10× repeat of HNF4_FOXA_v1 with poly-C/poly-G minimization and CpG minimization v1. In certain embodiment, the regulatory element comprising the 10× repeat of HNF4_FOXA_v1 with poly-C/poly-G minimization and CpG minimization v1 comprises SEQ ID NO: 32.

In some embodiments, the expression cassette comprises a regulatory element (e.g., an enhancer) comprising 10×repeat of HNF4_FOXA_v1 with poly-C/poly-G minimization v2. In certain embodiment, the regulatory element comprising the 10× repeat of HNF4_FOXA_v1 with poly-C/poly-G minimization v2 comprises SEQ ID NO: 33.

In some embodiments, the expression cassette comprises a regulatory element (e.g., an enhancer) comprising 10× repeat of HNF4_FOXA_v1 with poly-C/poly-G minimization and CpG minimization v2. In certain embodiment, the regulatory element comprising the 10× repeat of HNF4_FOXA_v1 with poly-C/poly-G minimization and CpG minimization v2 comprises SEQ ID NO: 34.

In some embodiments, the expression cassette comprises a regulatory element (e.g., an enhancer) comprising 10× repeat of HNF4_FOXA_v1 with poly-C/poly-G minimization v3. In certain embodiment, the regulatory element comprising the 10× repeat of HNF4_FOXA_v1 with poly-C/poly-G minimization v3 comprises SEQ ID NO: 35.

In some embodiments, the expression cassette comprises a regulatory element (e.g., an enhancer) comprising 10× repeat of HNF4_FOXA_v1 with poly-C/poly-G minimization and CpG minimization v3. In certain embodiment, the regulatory element comprising the 10× repeat of HNF4_FOXA_v1 with poly-C/poly-G minimization and CpG minimization v3 comprises SEQ ID NO: 36.

In some embodiments, the expression cassette comprises a regulatory element (e.g., an enhancer) comprising 10× repeat of the human SERPINA1 enhancer. In certain embodiment, the regulatory element comprising the 10× repeat of the human SERPINA1 enhancer comprises SEQ ID NO: 37.

In some embodiments, the expression cassette comprises a regulatory element (e.g., an enhancer) comprising 10× repeat of the Bushbaby SERPINA1 enhancer with adenenine nucleotide spacer. In certain embodiment, the regulatory element comprising the 10× repeat of the Bushbaby SERPINA1 enhancer with adenenine nucleotide spacer comprises SEQ ID NO: 38.

In some embodiments, the expression cassette comprises a regulatory element (e.g., an enhancer) comprising Bushbaby SERPINA1 enhancer, FOXA_HNF4_v1 enhancer, HNF4 consensus binding site enhancer. In certain embodiment, the regulatory element comprising the Bushbaby SERPINA1 enhancer, FOXA_HNF4_v1 enhancer, HNF4 consensus binding site enhancer comprises SEQ ID NO: 39.

In some embodiments, the expression cassette comprises a regulatory element (e.g., an enhancer) comprising HNF4 consensus binding site enhancer, Bushbaby SERPINA1 enhancer, FOXA_HNF4_v1 enhancer. In certain embodiment, the regulatory element comprising the HNF4 consensus binding site enhancer, Bushbaby SERPINA1 enhancer, FOXA_HNF4_v1 enhancer comprises SEQ ID NO: 40.

In some embodiments, the expression cassette comprises a regulatory element (e.g., an enhancer) comprising 3× repeat of hSerpEnh with 2mer spacers v1. In certain embodiment, the regulatory element comprising the 3× repeat of hSerpEnh with 2mer spacers v1 comprises SEQ ID NO: 41.

In some embodiments, the expression cassette comprises a regulatory element (e.g., an enhancer) comprising 3× repeat of hSerpEnh with 2mer spacers v2. In certain embodiment, the regulatory element comprising the 3× repeat of hSerpEnh with 2mer spacers v2 comprises SEQ ID NO: 42.

In some embodiments, the expression cassette comprises a regulatory element (e.g., an enhancer) comprising 3× repeat of hSerpEnh with 2mer spacers v3. In certain embodiment, the regulatory element comprising the 3× repeat of hSerpEnh with 2mer spacers v3 comprises SEQ ID NO: 43.

In some embodiments, the expression cassette comprises a regulatory element (e.g., an enhancer) comprising 3× repeat of hSerpEnh with 2mer spacers v4. In certain embodiment, the regulatory element comprising the 3× repeat of hSerpEnh with 2mer spacers v4 comprises SEQ ID NO: 44.

In some embodiments, the expression cassette comprises a regulatory element (e.g., an enhancer) comprising 3× repeat of hSerpEnh with 2mer spacers v5. In certain embodiment, the regulatory element comprising the 3× repeat of hSerpEnh with 2mer spacers v5 comprises SEQ ID NO: 45.

In some embodiments, the expression cassette comprises a regulatory element (e.g., an enhancer) comprising 3× repeat of hSerpEnh with 2mer spacers v6. In certain embodiment, the regulatory element comprising the 3× repeat of hSerpEnh with 2mer spacers v6 comprises SEQ ID NO: 46.

In some embodiments, the expression cassette comprises a regulatory element (e.g., an enhancer) comprising 3× repeat of hSerpEnh with 2mer spacers v7. In certain embodiment, the regulatory element comprising the 3× repeat of hSerpEnh with 2mer spacers v7 comprises SEQ ID NO: 47.

In some embodiments, the expression cassette comprises a regulatory element (e.g., an enhancer) comprising 3× repeat of hSerpEnh with 2mer spacers v8. In certain embodiment, the regulatory element comprising the 3× repeat of hSerpEnh with 2mer spacers v8 comprises SEQ ID NO: 48.

In some embodiments, the expression cassette comprises a regulatory element (e.g., an enhancer) comprising 3× repeat of hSerpEnh with 2mer spacers v9. In certain embodiment, the regulatory element comprising the 3× repeat of hSerpEnh with 2mer spacers v9 comprises SEQ ID NO: 49.

In some embodiments, the expression cassette comprises a regulatory element (e.g., an enhancer) comprising 3× repeat of hSerpEnh with 2mer spacers v10. In certain embodiment, the regulatory element comprising the 3× repeat of hSerpEnh with 2mer spacers v10 comprises SEQ ID NO: 50.

In some embodiments, the expression cassette comprises a regulatory element (e.g., an enhancer) comprising 3× repeat of hSerpEnh with 2mer spacers v11. In certain embodiment, the regulatory element comprising the 3× repeat of hSerpEnh with 2mer spacers v11comprises SEQ ID NO: 51.

In some embodiments, the expression cassette comprises a regulatory element (e.g., an enhancer) comprising 3× repeat of hSerpEnh with 2mer spacers v12. In certain embodiment, the regulatory element comprising the 3× repeat of hSerpEnh with 2mer spacers v12 comprises SEQ ID NO: 52.

In some embodiments, the expression cassette comprises a regulatory element (e.g., an enhancer) comprising 3× repeat of hSerpEnh with 2mer spacers v13. In certain embodiment, the regulatory element comprising the 3× repeat of hSerpEnh with 2mer spacers v13 comprises SEQ ID NO: 53.

In some embodiments, the expression cassette comprises a regulatory element (e.g., an enhancer) comprising 3× repeat of hSerpEnh with 2mer spacers v14. In certain embodiment, the regulatory element comprising the 3× repeat of hSerpEnh with 2mer spacers v14 comprises SEQ ID NO: 54.

In some embodiments, the expression cassette comprises a regulatory element (e.g., an enhancer) comprising 3× repeat of hSerpEnh with 2mer spacers v15. In certain embodiment, the regulatory element comprising the 3× repeat of hSerpEnh with 2mer spacers v15 comprises SEQ ID NO: 55.

In some embodiments, the expression cassette comprises a regulatory element (e.g., an enhancer) comprising 3× repeat of hSerpEnh with 2mer spacers v16. In certain embodiment, the regulatory element comprising the 3× repeat of hSerpEnh with 2mer spacers v16 comprises SEQ ID NO: 56.

In some embodiments, the expression cassette comprises a regulatory element (e.g., an enhancer) comprising 3× repeat of hSerpEnh with 2mer spacers v17. In certain embodiment, the regulatory element comprising the 3× repeat of hSerpEnh with 2mer spacers v17 comprises SEQ ID NO: 57.

In some embodiments, the expression cassette comprises a regulatory element (e.g., an enhancer) comprising 3× repeat of hSerpEnh with 2mer spacers v18. In certain embodiment, the regulatory element comprising the 3× repeat of hSerpEnh with 2mer spacers v18 comprises SEQ ID NO: 58.

In some embodiments, the expression cassette comprises a regulatory element (e.g., an enhancer) comprising 3× repeat of hSerpEnh with 2mer spacers v19. In certain embodiment, the regulatory element comprising the 3× repeat of hSerpEnh with 2mer spacers v19 comprises SEQ ID NO: 59.

In some embodiments, the expression cassette comprises a regulatory element (e.g., an enhancer) comprising 3× repeat of hSerpEnh with 2mer spacers v20. In certain embodiment, the regulatory element comprising the 3× repeat of hSerpEnh with 2mer spacers v20 comprises SEQ ID NO: 60.

In some embodiments, the expression cassette comprises a regulatory element (e.g., an enhancer) comprising 3× repeat of hSerpEnh with 3mer spacers v1. In certain embodiment, the regulatory element comprising the 3× repeat of hSerpEnh with 3mer spacers v1 comprises SEQ ID NO: 61.

In some embodiments, the expression cassette comprises a regulatory element (e.g., an enhancer) comprising 3× repeat of hSerpEnh with 3mer spacers v2. In certain embodiment, the regulatory element comprising the 3× repeat of hSerpEnh with 3mer spacers v2 comprises SEQ ID NO: 62.

In some embodiments, the expression cassette comprises a regulatory element (e.g., an enhancer) comprising 3× repeat of hSerpEnh with 3mer spacers v3. In certain embodiment, the regulatory element comprising the 3× repeat of hSerpEnh with 3mer spacers v3 comprises SEQ ID NO: 63.

In some embodiments, the expression cassette comprises a regulatory element (e.g., an enhancer) comprising 3× repeat of hSerpEnh with 5mer spacers v1. In certain embodiment, the regulatory element comprising the 3× repeat of hSerpEnh with 5mer spacers v1 comprises SEQ ID NO: 64.

In some embodiments, the expression cassette comprises a regulatory element (e.g., an enhancer) comprising 3× repeat of hSerpEnh with 5mer spacers v2. In certain embodiment, the regulatory element comprising the 3× repeat of hSerpEnh with 5mer spacers v2 comprises SEQ ID NO: 65.

In some embodiments, the expression cassette comprises a regulatory element (e.g., an enhancer) comprising 3× repeat of hSerpEnh with 5mer spacers v3. In certain embodiment, the regulatory element comprising the 3× repeat of hSerpEnh with 5mer spacers v3 comprises SEQ ID NO: 66.

In some embodiments, the expression cassette comprises a regulatory element (e.g., an enhancer) comprising 3× repeat of hSerpEnh with 11 mer spacers v1. In certain embodiment, the regulatory element comprising the 3× repeat of hSerpEnh with 11mer spacers v1 comprises SEQ ID NO: 67.

In some embodiments, the expression cassette comprises a regulatory element (e.g., an enhancer) comprising 3× repeat of hSerpEnh with 11 mer spacers v2. In certain embodiment, the regulatory element comprising the 3× repeat of hSerpEnh with 11mer spacers v2 comprises SEQ ID NO: 68.

In some embodiments, the expression cassette comprises a regulatory element (e.g., an enhancer) comprising 3× repeat of hSerpEnh with 11 mer spacers v3. In certain embodiment, the regulatory element comprising the 3× repeat of hSerpEnh with 11 mer spacers v3 comprises SEQ ID NO: 69.

In some embodiments, the expression cassette comprises a regulatory element (e.g., an enhancer) comprising 3× repeat of hSerpEnh with 11 mer spacers with HNF4 binding site in orientation 1 & FOXA binding site in orientation 1. In certain embodiment, the regulatory element comprising the 3× repeat of hSerpEnh with 11 mer spacers with HNF4 binding site in orientation 1 & FOXA binding site in orientation 1 comprises SEQ ID NO: 70.

In some embodiments, the expression cassette comprises a regulatory element (e.g., an enhancer) comprising 3× repeat of hSerpEnh with 11 mer spacers with HNF4 binding site in orientation 1 & FOXA binding site in orientation 2. In certain embodiment, the regulatory element comprising the 3× repeat of hSerpEnh with 11 mer spacers with HNF4 binding site in orientation 1 & FOXA binding site in orientation 2 comprises SEQ ID NO: 71.

In some embodiments, the expression cassette comprises a regulatory element (e.g., an enhancer) comprising 3× repeat of hSerpEnh with 11 mer spacers with HNF4 binding site in orientation 2 & FOXA binding site in orientation 1. In certain embodiment, the regulatory element comprising the 3× repeat of hSerpEnh with 11 mer spacers with HNF4 binding site in orientation 2 & FOXA binding site in orientation 1 comprises SEQ ID NO: 72.

In some embodiments, the expression cassette comprises a regulatory element (e.g., an enhancer) comprising 3× repeat of hSerpEnh with 11 mer spacers with HNF4 binding site in orientation 2 & FOXA binding site in orientation 2. In certain embodiment, the regulatory element comprising the 3× repeat of hSerpEnh with 11 mer spacers with HNF4 binding site in orientation 2 & FOXA binding site in orientation 2 comprises SEQ ID NO: 73.

In some embodiments, the expression cassette comprises a regulatory element (e.g., an enhancer) comprising 3× repeat of hSerpEnh with 30mer spacers v1. In certain embodiment, the regulatory element comprising the 3× repeat of hSerpEnh with 30mer spacers v1 comprises SEQ ID NO: 74.

In some embodiments, the expression cassette comprises a regulatory element (e.g., an enhancer) comprising 3× repeat of hSerpEnh with 30mer spacers v2. In certain embodiment, the regulatory element comprising the 3× repeat of hSerpEnh with 30mer spacers v2 comprises SEQ ID NO: 75.

In some embodiments, the expression cassette comprises a regulatory element (e.g., an enhancer) comprising 3× repeat of hSerpEnh with 30mer spacers v3. In certain embodiment, the regulatory element comprising the 3× repeat of hSerpEnh with 30mer spacers v3 comprises SEQ ID NO: 76.

In some embodiments, the expression cassette comprises a regulatory element (e.g., an enhancer) comprising 3× repeat of hSerpEnh with 30mer spacers with HNF4 binding site in orientation 1 & FOXA binding site in orientation 1. In certain embodiment, the regulatory element comprising the 3× repeat of hSerpEnh with 30mer spacers with HNF4 binding site in orientation 1 & FOXA binding site in orientation 1 comprises SEQ ID NO: 77.

In some embodiments, the expression cassette comprises a regulatory element (e.g., an enhancer) comprising 3× repeat of hSerpEnh with 30mer spacers with HNF4 binding site in orientation 1 & FOXA binding site in orientation 2. In certain embodiment, the regulatory element comprising the 3× repeat of hSerpEnh with 30mer spacers with HNF4 binding site in orientation 1 & FOXA binding site in orientation 2 comprises SEQ ID NO: 78.

In some embodiments, the expression cassette comprises a regulatory element (e.g., an enhancer) comprising 3× repeat of hSerpEnh with 30mer spacers with HNF4 binding site in orientation 2 & FOXA binding site in orientation 1. In certain embodiment, the regulatory element comprising the 3× repeat of hSerpEnh with 30mer spacers with HNF4 binding site in orientation 2 & FOXA binding site in orientation 1 comprises SEQ ID NO: 79.

In some embodiments, the expression cassette comprises a regulatory element (e.g., an enhancer) comprising 3× repeat of hSerpEnh with 30mer spacers with HNF4 binding site in orientation 2 & FOXA binding site in orientation 2. In certain embodiment, the regulatory element comprising the 3× repeat of hSerpEnh with 30mer spacers with HNF4 binding site in orientation 2 & FOXA binding site in orientation 2 comprises SEQ ID NO: 80.

In some embodiments, the expression cassette comprises a regulatory element (e.g., an enhancer) comprising 3 repeats of SERPINA1 enhancer derived from tibetan antelope, separated by T. In certain embodiment, the regulatory element comprising the 3× repeat of Tibetan antelope SERPINA1 comprises SEQ ID NO:138.

In some embodiments, the expression cassette comprises a regulatory element (e.g., an enhancer) comprising 3 repeats of SERPINA1 enhancer derived from armadillo with minimum CpG and separated by T. In certain embodiment, the regulatory element comprising the 3× repeat of Tibetan antelope SERPINA1 comprises SEQ ID NO:139.

In one embodiment, the disclosure provides an expression cassette comprising any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139.

In one embodiment, the expression cassette comprises a nucleic acid sequence that is at least 85%, 90%, 95%, 96%, 97%, 98%, or 99% identical to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139.

In one embodiment, the disclosure provides an expression cassette consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139.

The disclosed expression cassettes can be used in any situation where liver-specific transcription is desired. In various embodiments, any of the expression cassettes, including one or more of the enhancers, the spacers, the promoters, of the disclosure can be included in a viral vector (e.g., an AAV vector) or a non-viral vector (e.g., a ceDNA vector) for gene therapy methods in which liver-specific expression of a transgene is desired, such as liver-specific expression of a clotting factor (e.g., as described herein).

III. Viral vectors

In one embodiment, the disclosure relates to recombinant viral vectors comprising a nucleic acid sequence of a liver-specific promoter as described herein, in operative combination with a heterologous nucleic acid sequence encoding a therapeutic protein.

In one embodiment, the vector comprises a viral nucleic acid sequence of greater than 10, 20, 30, 40, 50, 100, or 200 nucleotides. In certain embodiments, the sequence of a viral nucleic acid comprises a human adeno-associated virus (hAAV) of serotypes 1, 2, 3B, 4, 5, 6, 7, 8, 9, or combinations or variants thereof, which it generally comprises an inverted terminal repeat of AAV.

In one embodiment, the disclosure provides a viral particle, e.g., a viral capsid comprising a vector as disclosed herein, e.g., wherein the vector is packaged in a capsid. The capsid can be a recombinant or chimeric capsid or particle, for example a capsid that has amino acid sequences that are a combination of AAV pseudotypes for VP 1, VP2 or VP3. An AAV capsid VP can be derived from a human AAVgene or animal AAV gene, or combinations with genetically modified alterations, i.e., AAV isolated from infected human cells or a non-human primate. Animal AAVs include those derived from birds, cattle, pigs, mice, etc. In one embodiment, the capsid may have amino acid sequences that are genetically modified or synthetic capsids identified by methods such as directed evolution or rational design.

In one embodiment, the vector is incompetent for replication within a human host, for example, the vector does not encode a viral polymerase.

In one embodiment, the liver-specific expression cassette comprises a sequence having at least 50, 60, 70, 75, 80, 85, 90, 91, 92, 93, 94, 95, 96, 97, 98, 99 or 100% sequence identity with any one of SEQ ID NOs: 1-80, 138 or 139 as set forth above.

Expression of a Protein from an AAV Vector

In one embodiment, the nucleic acid sequences and promoters of the disclosure are useful in the production of AAV vectors. AAV belongs to the Parvoviridae family and the Dependovirus genus. AAV is a small, enveloped virus that packages a single-stranded, linear DNA genome. Both the sense and antisense AAV DNA strands are packaged in AAV capsids with the same frequency.

The AAV genome is characterized by two inverted terminal repeats (ITRs) flanking two open reading frames (ORFs). In the AAV2 genome, for example, the first 125 nucleotides of the ITR are a palindrome, which folds back on itself to maximize base pairing and forms a T-shaped hairpin structure. The other 20 bases of the ITR, called sequence D, they remain unpaired. ITRs are cis-acting sequences important for AAV DNA replication; ITR is the origin of replication and serves as a primer for the synthesis of the second chain by DNA polymerase. The double-stranded DNA formed during this synthesis, which is called the replicating monomer, is used for a second round of self-priming replication and forms a replicating dimer. These double-stranded intermediates are processed using a chain-shifting mechanism, resulting in single-stranded DNA that is used for packaging and double-stranded DNA that is used for transcription. Located within the ITR are the Rep binding elements and a terminal resolution site (TRS). These characteristics are used by the viral regulatory protein Rep during AAV replication to process double-stranded intermediates. In addition to its role in AAV replication, ITR is also essential for AAV genome packaging, transcription, down-regulation under non-permissive conditions, and site-specific integration (Daya and Berns, Clin Microbiol Rev 21 (4): 583-593, 2008).

The AAV's left ORF contains the Rep gene, which encodes four proteins: Rep78, Rep 68, Rep52, and Rep40. The right ORF contains the Cap gene, which produces three viral capsid proteins (VP1, VP2, and VP3). The AAV capsid contains 60 viral capsid proteins arranged in icosahedral symmetry. VP1, VP2 and VP3 are present in a 1: 1: 10 molar ratio (Daya and Berns, Clin Microbiol Rev 21 (4): 583-593, 2008).

AAV vectors generally contain a transgene expression cassette between ITRs that replaces the rep and cap genes. Vector particles are produced by cotransfecting cells with a plasmid containing the vector genome and a packaging/helper construct that expresses the rep and cap proteins in trans. During infection, the genomes of AAV vectors enter the cell nucleus and can persist in multiple molecular states. A common result is the conversion of the AAV genome to a double-stranded circular episome by synthesis of the second strand or pairing with the complementary strand.

In the context of AAV vectors, the disclosed vectors generally have a recombinant genome that It comprises the following structure:

### (5′ITR of AAV)-(promoter)-(transgene)-(3′ITR of AAV)

As discussed above, these recombinant AAV vectors contain a transgene expression cassette between the ITRs that replaces the rep and cap genes. Vector particles are produced, for example, by cotransfecting cells with a plasmid containing the recombinant vector genome and a packaging/helper construct that expresses the rep and cap proteins in trans.

The AAV ITRs, and other selected AAV components described herein, can be readily selected from any AAV serotype, including, without limitation, AAV1, AAV2, AAV3, AAV4, AAV5, AAV6, AAV7, AAV8, AAV9 and function variants thereof. These ITRs or other AAV components can be easily isolated using techniques available to those skilled in the art from an AAV serotype. Said AAV can be isolated or obtained from academic, commercial or public sources (for example, the American Type Culture Collection, Manassas, Va.). Alternatively, AAV sequences can be obtained through synthetic means or other suitable means by reference to published sequences such as those available in the literature or in databases such as, for example, GenBank, PubMed or the like.

In one embodiment, the nucleic acids of the disclosure are part of an expression cassette or transgene. See for example, US Patent Application Publication 20150139953. The expression cassette is comprised of a transgene and regulatory sequences, eg, for example a promoter and 5′ and 3′ AVV inverted terminal repeats (ITRs). In a desirable embodiment, ITRs of AAV serotype 2 or 8 are used. However, ITRs can be selected from other suitable serotypes. An expression cassette is generally packaged in a capsid protein and delivered to a selected host cell.

In one embodiment, the disclosure provides a method of generating a recombinant adeno-associated virus (AAV) having an AAV serotype capsid, or a portion thereof. Such a method involves culturing a host cell that contains a nucleic acid sequence encoding an adeno-associated virus (AAV) serotype capsid protein; a functional rep gene; an expression cassette consisting of AAV inverted terminal repeats (ITRs) and a transgene; and enough ancillary functions to allow for packaging of the expression cassette into the AAV capsid protein. See for example, U.S. Patent Application Publication 20150139953.

Components for culture in the host cell to package an AAV expression cassette into an AAV capsid can be provided to the host cell in trans. Alternatively, one or more of the components (e.g., expression cassette, rep sequences, cap sequences, and/or helper functions) can be provided by a stable host cell that has been engineered to contain one or more of the components.

In one embodiment, the disclosure relates to recombinant vectors comprising a liver-specific promoter nucleic acid sequence of the disclosure in operative combination with the transgene. The transgene is a nucleic acid sequence, heterologous to the vector sequences that flank the transgene, that encodes a protein, e.g., a therapeutic protein, or other product of interest. The nucleic acid coding sequence is operably linked to regulatory components in a manner that allows transcription, translation and/or transgene expression in a host cell.

A typical transgene is a sequence that encodes a product that is useful in biology and medicine, such as proteins, peptides, RNA, enzymes, dominant negative mutants, or catalytic RNA. Desirable RNA molecules include mRNA, tRNA, dsRNA, ribosomal RNA, catalytic RNA, siRNA, guide RNA (gRNA), microRNA, small hairpin RNA, trans-splice RNA, and antisense RNA. An example of a useful RNA sequence is a sequence that inhibits or extinguishes the expression of a targeted nucleic acid sequence in the treated animal.

The transgene can be used to correct or improve genetic deficiencies, which may include deficiencies in which normal genes are expressed at lower than normal levels or deficiencies in which the functional gene product is not expressed. A preferred type of transgenic sequence encodes a therapeutic protein or polypeptide that is expressed in a host cell. The disclosure further contemplates the use of multiple transgenes, for example, to correct or improve a genetic defect caused by a multi-subunit protein. In certain situations, a different transgene can be used to encode each subunit of a protein, or to encode different peptides or proteins. This is desirable when the size of the DNA encoding the protein subunit is large, for example, for an immunoglobulin, platelet-derived growth factor, or a dystrophin protein. In order for the cell to produce the multi-subunit protein, a cell is infected with the recombinant virus that contains each of the different subunits. Alternatively, different subunits of a protein may be encoded by the same transgene.

The expression cassette can be carried in any suitable viral vector which is supplied to a host cell. The plasmids useful in the present disclosure can be engineered to be suitable for replication and, optionally, integration in prokaryotic cells, mammalian cells, or both. These plasmids (or other vectors carrying the AAV 5′ ITR-heterologous molecule-3′ ITR) contain sequences that allow replication of the expression cassette in eukaryotes and/or prokaryotes and selection markers for these systems. Preferably, the molecule that carries the expression cassette is transfected into the cell, where it may exist transiently. Alternatively, the expression cassette (carrying the 5′ITR of AAV-heterologous molecule-3′ ITR) can be stably integrated into the genome of the host cell, either chromosomally or as an episome. In certain embodiments, the expression cassette may be present in multiple copies, optionally in head-to-head, head-to-tail, or tail-to-tail concatamers. Suitable transfection techniques are known and can be easily used to deliver the expression cassette to the host cell.

In general, when the vector comprising the expression cassette is delivered by transfection, the vector and the relative amounts of vector DNA can be adjusted to the host cells, taking into account factors such as the selected vector, the delivery method and selected host cells. In addition to the expression cassette, the host cell contains the sequences that drive the expression of the AAV capsid protein in the host cell and the rep sequences of the same serotype as the AAV ITR serotype found in the expression cassette, or a cross-complement serotype. Although the molecules that provide rep and cap may exist in the host cell transiently (i.e., through transfection), it is preferred that one or both of the rep and cap proteins and the promoter (s) that control their expression they are stably expressed in the host cell, for example, as an episome or by integration into the chromosome of the host cell.

The packaging host cell generally also contains helper functions for packaging the rAAV of the disclosure. Optionally, these functions can be supplied by a herpesvirus. More desirably, the necessary auxiliary functions are each provided from a source of human or non-human primate adenoviruses, such as those described above and/or available from a variety of sources, including the American Type Culture Collection (ATCC), Manassas, Va. (USA). The desired auxiliary functions can be provided using any means that allows their expression in a cell.

Introduction into the vector host cell can be accomplished by any means known in the art or as disclosed above, including transfection, infection, electroporation, liposome delivery, membrane fusion techniques, high-speed DNA coated microgranules, infection viral or protoplast fusion, among others. One or more of the adenoviral genes can be stably integrated into the genome of the host cell, stably expressed as episomes, or transiently expressed. All gene products can be expressed transiently, at an episome, or stably integrated, or some of the gene products can be stably expressed while others are transiently expressed. Furthermore, promoters for each of the adenoviral genes can be independently selected from a constitutive promoter, an inducible promoter, or a natural adenoviral promoter. Promoters may be regulated by a specific physiological state of the organism or the cell (i.e., by the state of differentiation or in replicating or quiescent cells) or by exogenously added factors, for example.

The introduction of the molecules (such as plasmids or viruses) into the host cell can be accomplished using techniques known to the person skilled in the art. In a preferred embodiment, conventional transfection techniques, eg, transfection or electroporation with CaPO4, and/or infection by adenovirus/AAV hybrid vectors are used in cell lines such as the HEK 293 human embryonic kidney cell line (a cell line human kidney containing functional adenovirus E1 genes that provide trans-acting E1 proteins).

One of skill in the art will readily understand that AAV techniques can be adapted for use in these and other viral vector systems for gene delivery in vitro, ex vivo, or in vivo. In certain embodiments, the disclosure contemplates the use of nucleic acids and vectors disclosed herein in a variety of rAAV and non-rAAV vector systems. Such vector systems can include, for example, lentiviruses, retroviruses, poxviruses, vaccinia viruses, and adenoviral systems, among others.

In certain embodiments, the protein is a fVIII or fIX or a variant thereof as described herein. In certain embodiments, the codon and promoter optimization schemes disclosed herein could be used for any gene therapy with AAV targeting the liver. Other metabolic diseases caused by liver enzyme deficiencies and the expression of these functional proteins are contemplated.

In certain embodiments the nucleic acid sequence encoding a therapeutic protein comprises codons that are used or differentially represented in highly expressed genes within the liver or other specific tissue compared to the use of codons from the entire coding region of the human genome and avoid codons that are underrepresented in the liver or other specific tissue.

### IV. Non-Viral Vectors

In one embodiment, the expression cassettes described herein are useful in the production of non-vectors.

In one embodiment, the expression cassettes described herein are useful in the production of ceDNA vectors. In one embodiment, the disclosure provides the expression and/or production of a therapeutic protein (e.g., a liver-specific protein, e.g., a FVIII protein) in a cell, e.g., a liver cell, from a non-viral DNA vector, e.g., a ceDNA vector as described herein. In particular, ceDNA vectors for expression of a therapeutic protein (e.g., a FVIII protein) comprise a pair of ITRs (e.g., symmetric or asymmetric as described herein) and between the ITR pair, a nucleic acid encoding a therapeutic protein (e.g., a FVIII protein) operatively linked to a promoter or regulatory sequence. A distinct advantage of ceDNA vectors for expression of a therapeutic protein (e.g., a FVIII protein) over traditional AAV vectors, and even lentiviral vectors, is that there is no size constraint for the nucleic acid sequences, e.g., heterologous nucleic acid sequences, encoding a desired protein. Even a full length 6.8 kb FVIII protein can be expressed from a single ceDNA vector. Thus, ceDNA vectors described herein can be used to express a therapeutic FVIII protein in a subject in need thereof, e.g., a subject with hemophilia A.

In general, a ceDNA vector for expression of a therapeutic protein as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

As one will appreciate, the ceDNA vector technologies can be adapted to any level of complexity or can be used in a modular fashion, where expression of different components of a therapeutic protein (e.g., a FVIII protein) can be controlled in an independent manner. For example, it is specifically contemplated that the ceDNA vector technologies described here can be as simple as using a single ceDNA vector to express a single gene sequence a therapeutic protein (e.g., a FVIII protein) or can be as complex as using multiple ceDNA vectors, where each vector expresses multiple FVIII therapeutic proteins or associated co-factors or accessory proteins that are each independently controlled by different promoters. The following embodiments are specifically contemplated and can adapted by one of skill in the art as desired.

In one embodiment, a single ceDNA vector can be used to express a single component of a ntherapeutic protein (e.g., a FVIII protein). Alternatively, a single ceDNA vector can be used to express multiple components (e.g., at least 2) of a therapeutic protein (e.g., a FVIII protein) under the control of a single promoter (e.g., a strong promoter), optionally using an IRES sequence(s) to ensure appropriate expression of each of the components, e.g., co-factors or accessory proteins.

As one of skill in the art will appreciate, it is often desirable to express components of a therapeutic protein (e.g., a FVIII protein) at different expression levels, thus controlling the stoichiometry of the individual components expressed to ensure efficient protein folding and combination in the cell. Additional variations of ceDNA vector technologies can be envisioned by one of skill in the art or can be adapted from protein production methods using conventional vectors.

Certain methods for the production of a ceDNA vector for expression of a therapeutic protein (e.g., a FVIII protein) comprising an asymmetrical ITR pair or symmetrical ITR pair as defined herein is described in section IV of International application PCT/US18/49996 filed Sep. 7, 2018, which is incorporated herein in its entirety by reference. In some embodiments, a ceDNA vector for expression of a therapeutic protein (e.g., a FVIII protein) as disclosed herein can be produced using insect cells, as described herein. In alternative embodiments, a ceDNA vector for expression of a therapeutic protein (e.g., a FVIII protein) as disclosed herein can be produced synthetically and in some embodiments, in a cell-free method, as disclosed on International Application PCT/US19/14122, filed Jan. 18, 2019, which is incorporated herein in its entirety by reference.

As described herein, in one embodiment, a ceDNA vector for expression of a therapeutic protein (e.g., a FVIII protein) can be obtained, for example, by the process comprising the steps of: a) incubating a population of host cells (e.g., insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g., AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the disclosure provides for use of host cell lines that have stably integrated the DNA vector polynucleotide expression template (ceDNA template) into their own genome in production of the non-viral DNA vector, e.g., as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors for expression of a therapeutic protein (e.g., a FVIII protein) as described herein are insect cells, and baculovirus is used to deliver both the polynucleotide that encodes Rep protein and the non-viral DNA vector polynucleotide expression construct template for ceDNA. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector for expression of a therapeutic protein (e.g., a FVIII protein) can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA.

ceDNA Plasmid

A ceDNA-plasmid is a plasmid used for later production of a ceDNA vector for expression of a therapeutic protein (e.g., a FVIII protein). In some embodiments, a ceDNA-plasmid can be constructed using known techniques to provide at least the following as operatively linked components in the direction of transcription: (1) a modified 5′ ITR sequence; (2) an expression cassette as described herein comprising any one of SEQ ID NOs: 1-80, 138 and 139 and comprising a therapeutic transgene; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector for expression of therapeutic protein (e.g., a FVIII protein) is obtained from a plasmid, referred to herein as a “ceDNA-plasmid” encoding in this order: a first adeno-associated virus (AAV) inverted terminal repeat (ITR), an expression cassette as described herein comprising any one of SEQ ID NsS: 1-80, 138 and 139 and comprising a therapeutic transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette as described herein comprising any one of SEQ ID NOs: 1-80, 138 and 139 and comprising a therapeutic transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the ceDNA-plasmid system is devoid of viral capsid protein coding sequences (i.e., it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present disclosure can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. e.g., NCBI: NC 002077; NC 001401; NC001729; NC001829; NC006152; NC 006260; NC 006261; Kotin and Smith, The Springer Index of Viruses, available at the URL maintained by Springer. In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAVO) vector for expression of a therapeutic protein (e.g., a FVIII protein) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

Exemplary Method of Making the ceDNA Vectors from ceDNA Plasmids

Methods for making capsid-less ceDNA vectors for expression of a therapeutic protein (e.g., a FVIII protein) are also provided herein, notably a method with a sufficiently high yield to provide sufficient vector for in vivo experiments.

In some embodiments, a method for the production of a ceDNA vector for expression of a therapeutic protein (e.g., a FVIII protein) comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### Cell Lines

Host cell lines used in the production of a ceDNA vector for expression of a therapeutic protein (e.g., a FVIII protein) can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

ceDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

Isolating and Purifying ceDNA vectors

ceDNA-vectors for expression of a therapeutic protein (e.g., a FVIII protein) disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode a therapeutic protein (e.g., a FVIII protein), or plasmids encoding one or more REP proteins.

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for expression of a therapeutic protein (e.g., a FVIII protein) are described herein. Expression constructs used for generating a ceDNA vector for expression of a therapeutic protein (e.g., a FVIII protein) as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for expression of a therapeutic protein (e.g., a FVIII protein) can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for expression of a therapeutic protein (e.g., a FVIII protein) as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The capsid-free AAV vector is then recovered by, e.g., precipitation.

In some embodiments, ceDNA vectors for expression of a therapeutic protein (e.g., a FVIII protein) can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membrane proteins recognized by specific receptors on the respective cell types. (See also EP 10306226)

Another aspect of the disclosure herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

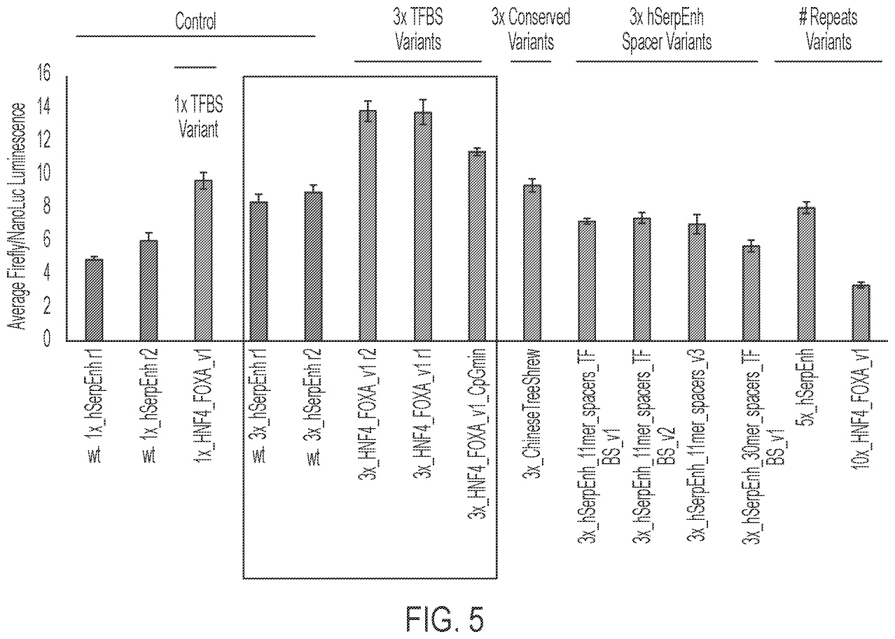

FIG. 5 of International application PCT/US18/49996 shows a gel confirming the production of ceDNA from multiple ceDNA-plasmid constructs using the method described in the Examples.

### V. Exemplary Recombinant Vectors

The nucleic acid sequences disclosed herein are useful in the production of expression plasmid, viral (AAV and rAAV) and non-viral vectors (ceDNA), and are also useful as antisense delivery vectors, gene therapy vectors, gene editing vectors (gRNA), or vaccine vectors.

In one embodiment, the disclosure provides a viral gene delivery vector comprising any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138 or SEQ ID NO: 139 operably linked to a liver-specific promoter and a therapeutic transgene. In one embodiment, the disclosure provides a viral gene delivery vector comprising a nucleic acid sequence that is at least 85%, 90%, 95%, 96%, 97%, 98%, or 99% identical to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139 operably linked to a liver-specific promoter and a therapeutic transgene. In one embodiment, the disclosure provides a viral gene delivery vector consisting of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139 operably linked to a liver-specific promoter and a therapeutic transgene. In one embodiment, the disclosure provides a non-viral gene delivery vector comprising any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139 operably linked to a liver-specific promoter and a therapeutic transgene. In one embodiment, the disclosure provides a non-viral gene delivery vector comprising a nucleic acid sequence that is at least 85%, 90%, 95%, 96%, 97%, 98%, or 99% identical to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139 operably linked to a liver-specific promoter and a therapeutic transgene. In one embodiment, the disclosure provides a non-viral gene delivery vector consisting of comprising any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 138, or SEQ ID NO: 139 operably linked to a liver-specific promoter and a therapeutic transgene.

In one embodiment, the nucleic acids of the disclosure can be part of any genetic element (vector) that can be supplied to a host cell, for example, naked DNA, a plasmid, phage, transposon, cosmid, episome, a protein in a non-viral delivery vehicle (e.g., a lipid-based transporter), viruses, etc. that transfer the sequences carried on them.

In one embodiment, a vector can be a lentivirus-based vector (containing genes or lentiviral sequences), for example, having nucleic acid sequences derived from VSVG or GP64 pseudotypes or both.

According to some aspects, the disclosure refers to virus particles, e.g., capsids, that contain the nucleic acid sequences encoding the expression cassettes and proteins disclosed herein. Viral particles, capsids, and recombinant vectors are useful in delivering a heterologous gene or other nucleic acid sequences to a target cell. Nucleic acids can be easily used in a variety of vector systems, capsids, and host cells. In one embodiment, the nucleic acids are in vectors contained within a capsid comprising terminal protection proteins, including AAV capsid proteins vp1, vp2, vp3 and hypervariable regions.

### Exemplary Therapeutic Protein (e.g., a FVIII Protein)

In particular, a ceDNA vector for expression of a therapeutic protein (e.g., a FVIII protein) as disclosed herein can encode, for example, but is not limited to a FVIII protein, as well as variants, and/or active fragments thereof, for use in the treatment, prophylaxis, and/or amelioration of one or more symptoms of hemophilia A. In one aspect, the hemophilia A is a human hemophilia A.

### FVIII Therapeutic Proteins and Fragments Thereof

Essentially any version of the FVIII therapeutic protein or fragment thereof (e.g., functional fragment) can be encoded by and expressed in and from a viral or non-viral vector as described herein. One of skill in the art will understand that FVIII therapeutic protein includes all splice variants and orthologs of the Therapeutic protein (e.g., a FVIII protein). FVIII therapeutic protein includes intact molecules as well as fragments (e.g., functional) thereof.

In one embodiment, the nucleic acid sequence encoding the protein comprises a higher percentage of liver cell specific amino acid codons compared to the general use of human codons. According to some aspects, the disclosure provides methods of treating a subject diagnosed with a genetic disease or disorder that results in the expression of a mutated or truncated non-functional protein by administering an effective amount of a vector disclosed herein (e.g., an AAV vector or a ceDNA vector) to express a functional liver protein.

### Factor VIII

Factor VIII is the nonenzymatic cofactor to the activated clotting factor IX (FIXa), which, when proteolytically activated, interacts with FIXa to form a tight noncovalent complex that binds to and activates factor X (FX).

The Factor VIII gene or protein can also be referred to as F8, Coagulation Factor VIII, Procoagulant Component, Antihemophilic Factor, F8C, AHF, DXS1253E, FVIII, HEMA, or F8B. Expression of the Factor VIII gene is tissue-specific and is mostly observed in liver cells. The highest level of the mRNA and Factor VIII proteins has been detected in liver sinusoidal cells; significant amounts of Factor VIII are also present in hepatocytes and in Kupffer cells (resident macrophages of liver sinusoids). Moderate levels of Factor VIII protein are detectable in the serum and plasma. Low to moderate levels of Factor VIII protein are expressed in fetal brain, retina, kidney and testis.

Factor VIII mRNA is expressed throughout many tissues of the body, including bone marrow, whole blood, white blood cells, lymph nodes, thymus, brain, cerebral cortex, cerebellum, retina, spinal cord, tibial nerve, heart, artery, smooth muscle, skeletal muscle, small intestine, colon, adipocytes, kidney, liver, lung, spleen, stomach, esophagus, bladder, pancreas, thyroid, salivary gland, adrenal gland, pituitary gland, breast, skin, ovary, uterus, placenta, prostate, and testis. The FVIII gene localized on the long arm of the X chromosome occupies a region approximately 186 kbp long and consists of 26 exons (69-3,106 bp) and introns (from 207 bp to 32.4 kbp). The total length of the coding sequence of this gene is 9 kbp.

The mature factor VIII polypeptide comprises the A1-A2-B-A3-C1-C2 structural domains. Three acidic subdomains, which are denoted as a1-a3-A1(a1)-A2(a2)-B-(a3)A3-C1-C2, localize at the boundaries of A domains and play a significant role in the interaction between FVIII and other proteins (in particular, with thrombin). Mutations in these subdomains reduce the level of factor VIII activation by thrombin.

The factor VIII protein (Coagulation factor VIII isoform) is a preproprotein [Homo sapiens]; Accession number: NP_000123.1 (2351 aa) and has the following sequence:

(SEQ ID NO: 142)
MQIELSTCFFLCLLRFCFSATRRYYLGAVELSWDYMQSDLGELPVDARFPPRVPKSFPFNTSVVYKKTL
FVEFTDHLENIAKPRPPWMGLLGPTIQAEVYDTVVITLKNMASHPVSLHAVGVSYWKASEGAEYDDQTS
QREKEDDKVFPGGSHTYVWQVLKENGPMASDPLCLTYSYLSHVDLVKDLNSGLIGALLVCREGSLAKEK
TQTLHKFILLFAVFDEGKSWHSETKNSLMQDRDAASARAWPKMHTVNGYVNRSLPGLIGCHRKSVYWHV
IGMGTTPEVHSIFLEGHTFLVRNHRQASLEISPITFLTAQTLLMDLGQFLLFCHISSHQHDGMEAYVKV
DSCPEEPQLRMKNNEEAEDYDDDLTDSEMDVVRFDDDNSPSFIQIRSVAKKHPKTWVHYIAAEEEDWDY
APLVLAPDDRSYKSQYLNNGPQRIGRKYKKVRFMAYTDETFKTREAIQHESGILGPLLYGEVGDTLLII
FKNQASRPYNIYPHGITDVRPLYSRRLPKGVKHLKDFPILPGEIFKYKWTVTVEDGPTKSDPRCLTRYY
SSFVNMERDLASGLIGPLLICYKESVDQRGNQIMSDKRNVILFSVFDENRSWYLTENIQRFLPNPAGVQ
LEDPEFQASNIMHSINGYVEDSLQLSVCLHEVAYWYILSIGAQTDFLSVFFSGYTFKHKMVYEDILTLF
PFSGETVFMSMENPGLWILGCHNSDERNRGMTALLKVSSCDKNIGDYYEDSYEDISAYLLSKNNAIEPR
SFSQNSRHPSTRQKQFNATTIPENDIEKTDPWFAHRTPMPKIQNVSSSDLLMLLRQSPTPHGLSLSDLQ
EAKYETFSDDPSPGAIDSNNSLSEMTHFRPQLHHSGDMVFTPESGLQLRLNEKLGTTAATELKKLDFKV
SSTSNNLISTIPSDNLAAGTDNTSSLGPPSMPVHYDSQLDTTLFGKKSSPLTESGGPLSLSEENNDSKL
LESGLMNSQESSWGKNVSSTESGRLFKGKRAHGPALLTKDNALFKVSISLLKINKTSNNSATNRKTHID
GPSLLIENSPSVWQNILESDTEFKKVTPLIHDRMLMDKNATALRLNHMSNKTTSSKNMEMVQQKKEGPI
PPDAQNPDMSFFKMLFLPESARWIQRTHGKNSLNSGQGPSPKQLVSLGPEKSVEGQNFLSEKNKVVVGK
GEFTKDVGLKEMVFPSSRNLFLINLDNLHENNTHNQEKKIQEEIEKKETLIQENVVLPQIHTVTGTKNF
MKNLFLLSTRQNVEGSYDGAYAPVLQDFRSLNDSTNRTKKHTAHFSKKGEEENLEGLGNQTKQIVEKYA
CTTRISPNTSQQNFVTQRSKRALKQFRLPLEETELEKRIIVDDTSTQWSKNMKHLTPSTLTQIDYNEKE
KGAITQSPLSDCLTRSHSIPQANRSPLPIAKVSSFPSIRPIYLTRVLFQDNSSHLPAASYRKKDSGVQE
SSHFLQGAKKNNLSLAILTLEMTGDQREVGSLGTSATNSVTYKKVENTVLPKPDLPKTSGKVELLPKVH
IYQKDLFPTETSNGSPGHLDLVEGSLLQGTEGAIKWNEANRPGKVPFLRVATESSAKTPSKLLDPLAWD
NHYGTQIPKEEWKSQEKSPEKTAFKKKDTILSLNACESNHAIAAINEGQNKPEIEVTWAKQGRTERLCS
QNPPVLKRHQREITRTTLQSDQEEIDYDDTISVEMKKEDEDIYDEDENQSPRSFQKKTRHYFIAAVERL
WDYGMSSSPHVLRNRAQSGSVPQFKKVVFQEFTDGSFTQPLYRGELNEHLGLLGPYIRAEVEDNIMVTF
RNQASRPYSFYSSLISYEEDQRQGAEPRKNFVKPNETKTYFWKVQHHMAPTKDEFDCKAWAYFSDVDLE
KDVHSGLIGPLLVCHTNTLNPAHGRQVTVQEFALFFTIFDETKSWYFTENMERNCRAPCNIQMEDPTFK
ENYRFHAINGYIMDTLPGLVMAQDQRIRWYLLSMGSNENIHSIHFSGHVFTVRKKEEYKMALYNLYPGV
FETVEMLPSKAGIWRVECLIGEHLHAGMSTLFLVYSNKCQTPLGMASGHIRDFQITASGQYGQWAPKLA
RLHYSGSINAWSTKEPFSWIKVDLLAPMIIHGIKTQGARQKFSSLYISQFIIMYSLDGKKWQTYRGNST
GTLMVFFGNVDSSGIKHNIFNPPIIARYIRLHPTHYSIRSTLRMELMGCDLNSCSMPLGMESKAISDAQ
ITASSYFTNMFATWSPSKARLHLQGRSNAWRPQVNNPKEWLQVDFQKTMKVTGVTTQGVKSLLTSMYVK
EFLISSSQDGHQWTLFFQNGKVKVFQGNQDSFTPVVNSLDPPLLTRYLRIHPQSWVHQIALRMEVLGCE
AQDLY

In one embodiment, FVIII therapeutic protein can be an “therapeutic protein variant,” which refers to the FVIII therapeutic protein having an altered amino acid sequence, composition or structure as compared to its corresponding native FVIII therapeutic protein. In one embodiment, FVIII is a functional version (e.g., wild type Therapeutic protein (e.g., a FVIII protein)). It may also be useful to express a mutant version of Therapeutic protein (e.g., a FVIII protein) such as a point mutation (F309 mutation) or deletion mutation (e.g., B domain deleted and/or single chain recombinant FVIII) as described in many examples herein. FVIII therapeutic protein expressed from the ceDNA vectors may further comprise a sequence/moiety that confers an additional functionality, such as fluorescence, enzyme activity, or secretion signal. In one embodiment, an FVIII therapeutic protein variant comprises a non-native tag sequence for identification (e.g., an immunotag) to allow it to be distinguished from endogenous FVIII therapeutic protein in a recipient host cell.

It is well within the abilities of one of skill in the art to take a known and/or publicly available protein sequence of e.g., FVIII therapeutic protein and reverse engineer a cDNA sequence to encode such a protein. The cDNA can then be codon optimized to match the intended host cell and inserted into a vector as described herein.

In one embodiment, the FVIII therapeutic protein encoding sequence can be derived from an existing host cell or cell line, for example, by reverse transcribing mRNA obtained from the host and amplifying the sequence using PCR.

### Vectors Expressing FVIII Proteins

A ceDNA vector having one or more sequences encoding a desired FVIII therapeutic protein can comprise regulatory sequences such as promoters, secretion signals, introns, polyA regions, and enhancers to maximize expression of the FVIII therapeutic protein when delivered to a desired cell or tissue. At a minimum, a ceDNA vector comprises one or more nucleic acid sequences encoding the FVIII therapeutic protein or functional fragment thereof.

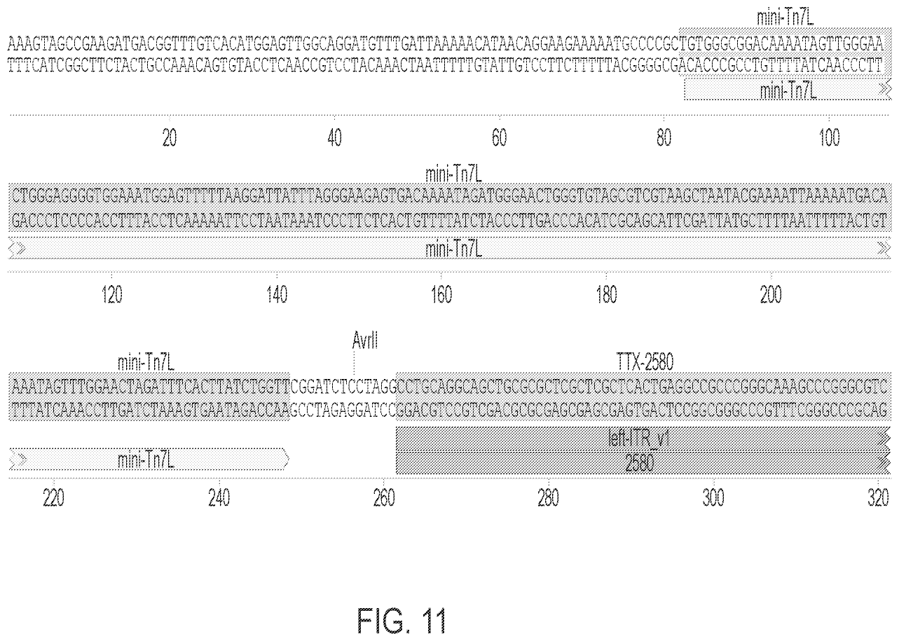

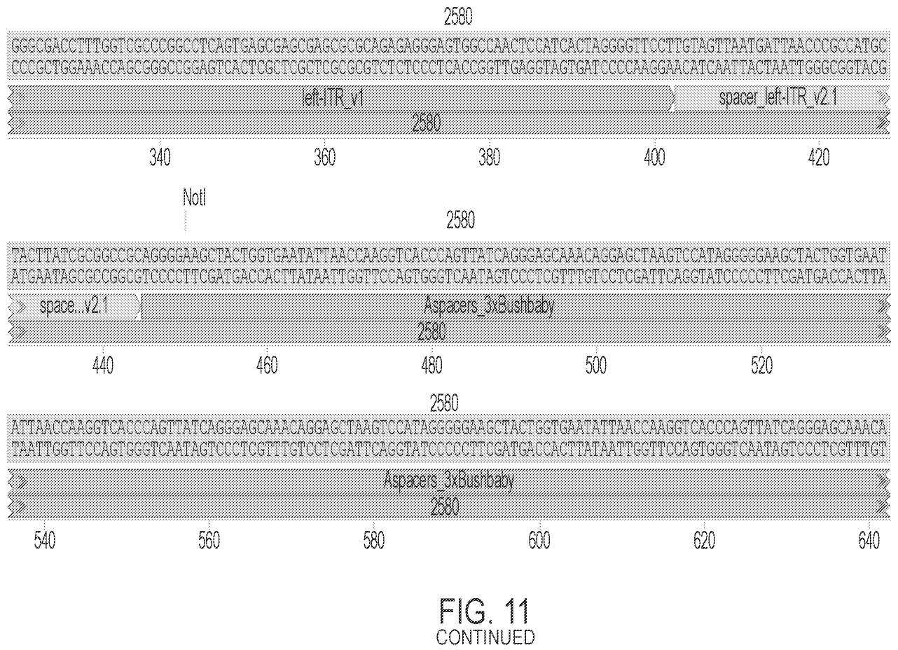

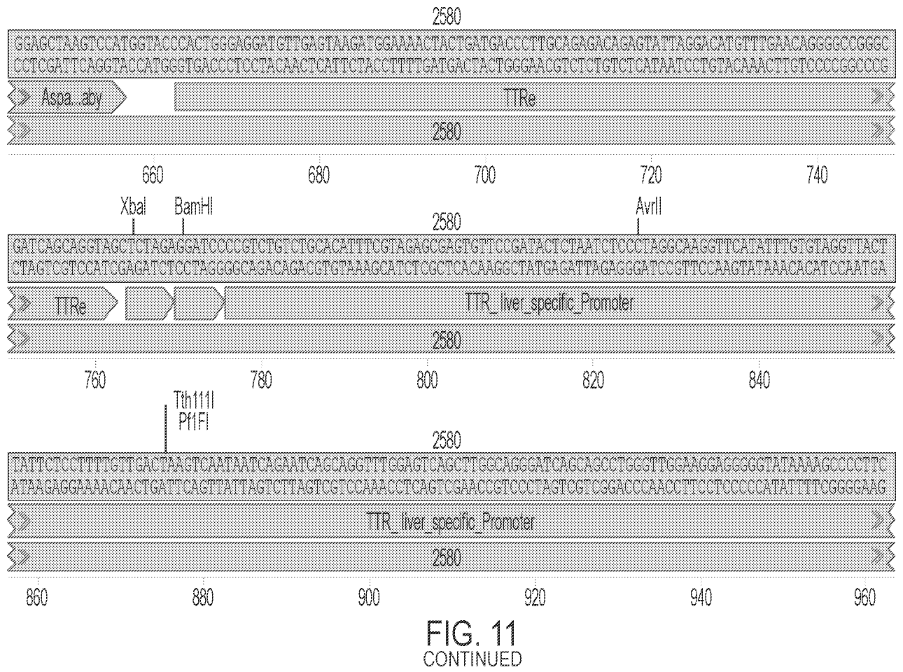

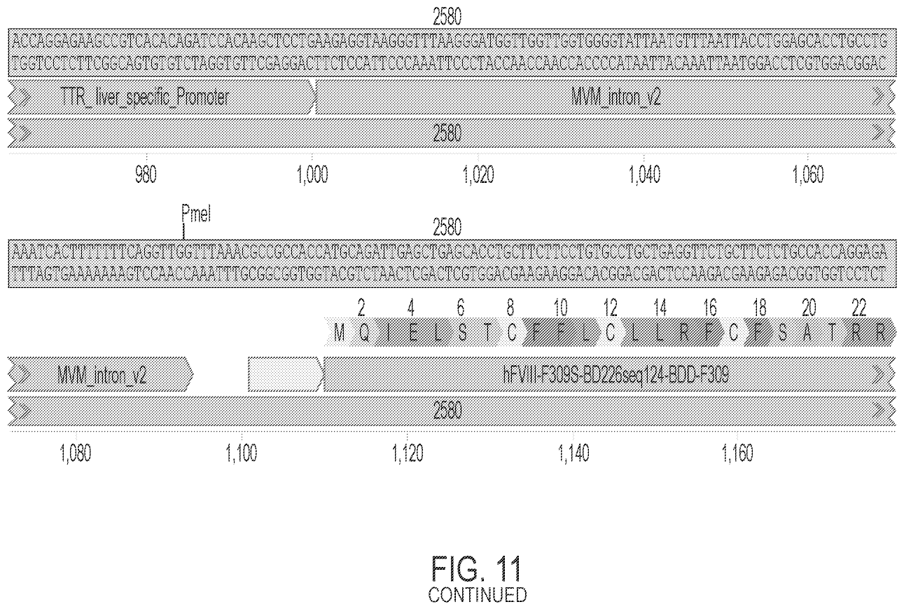

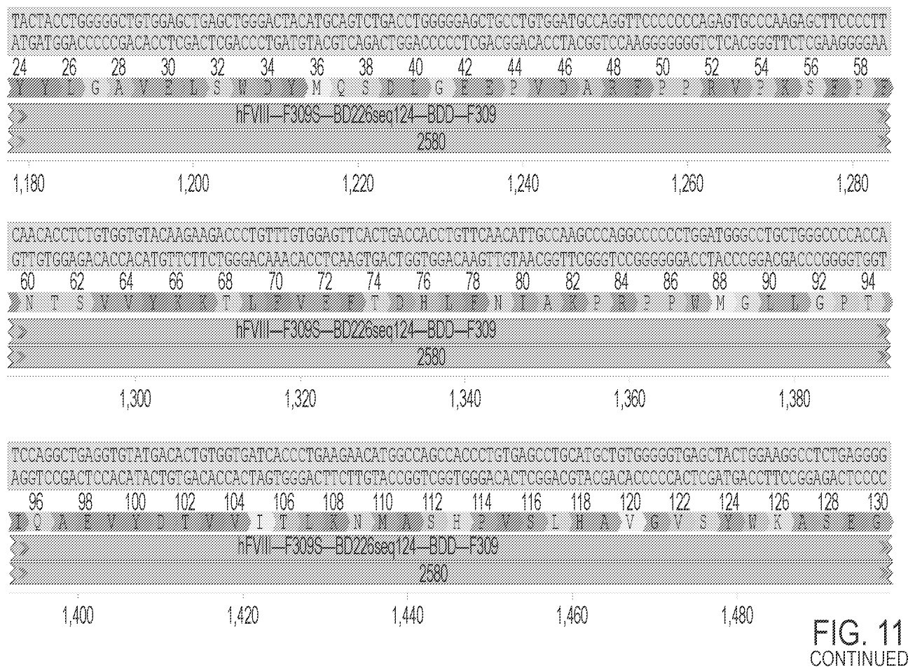

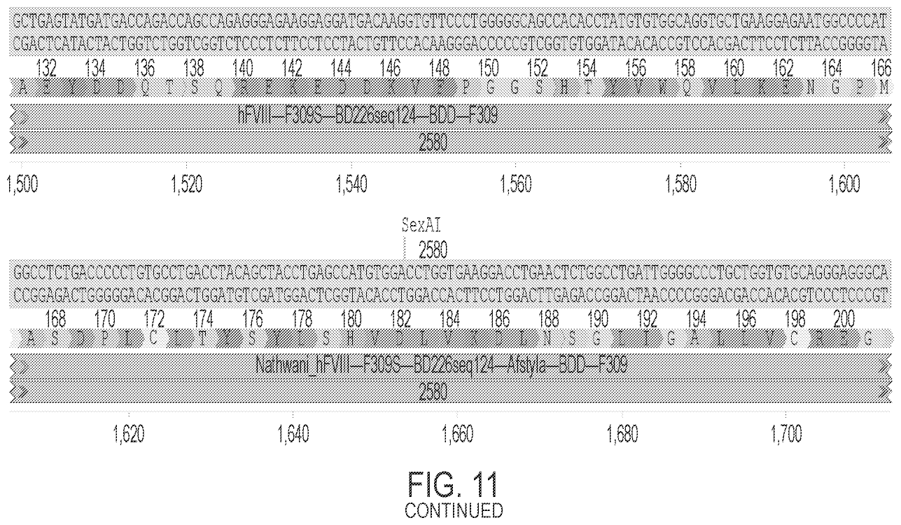

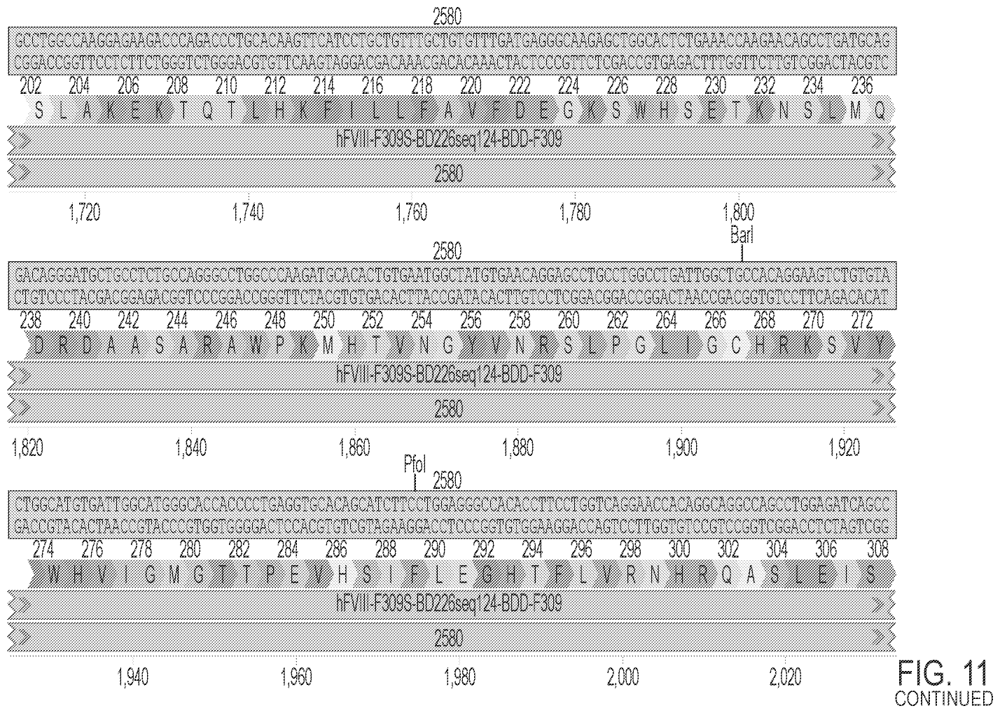

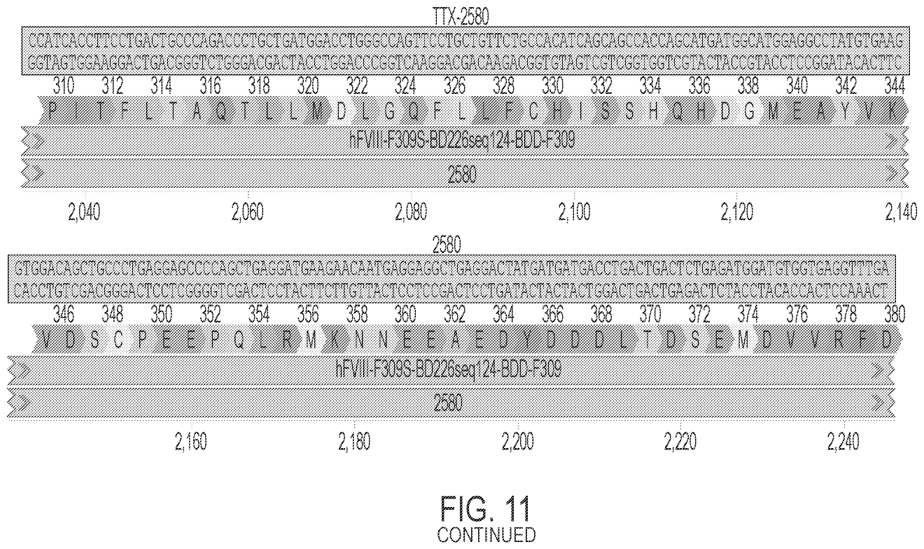

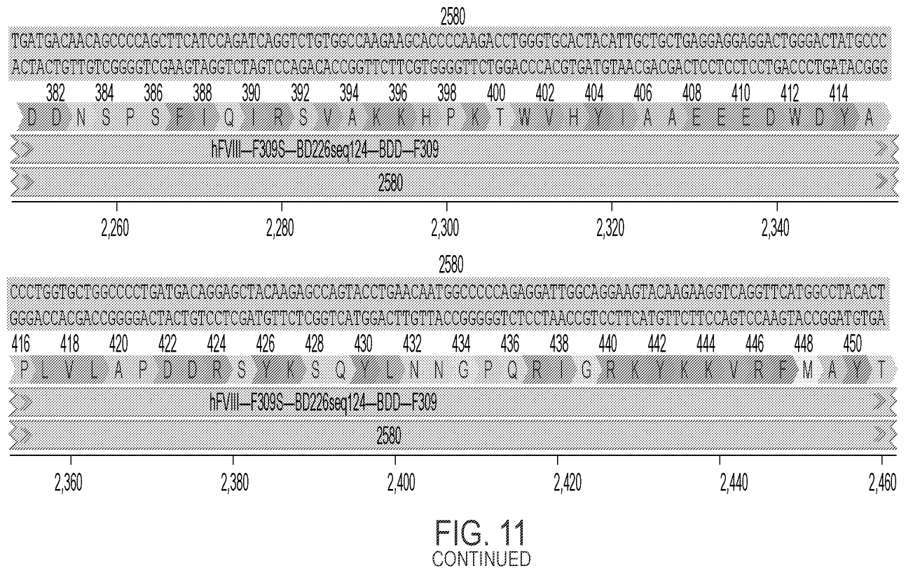

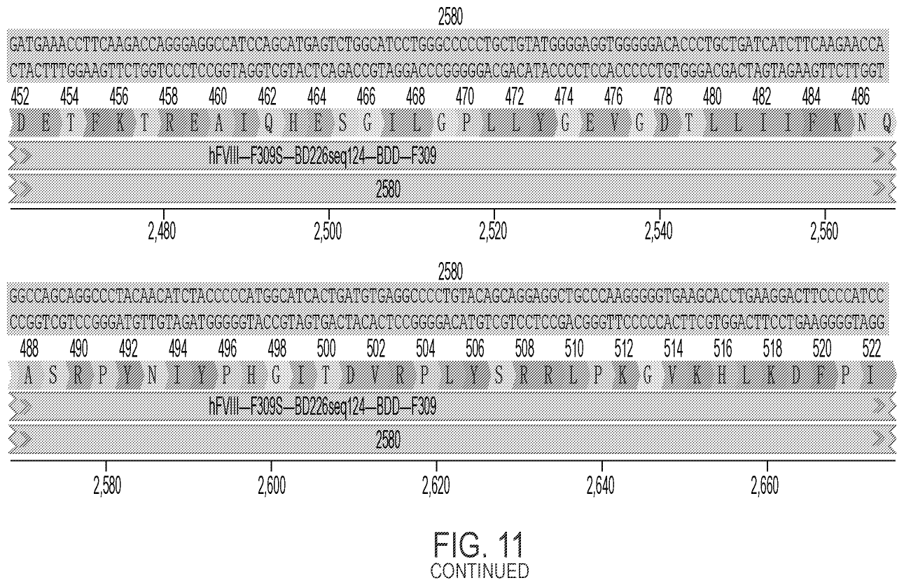

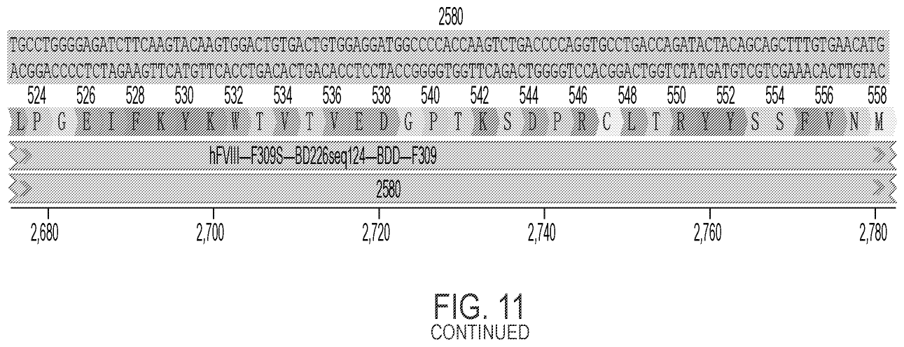

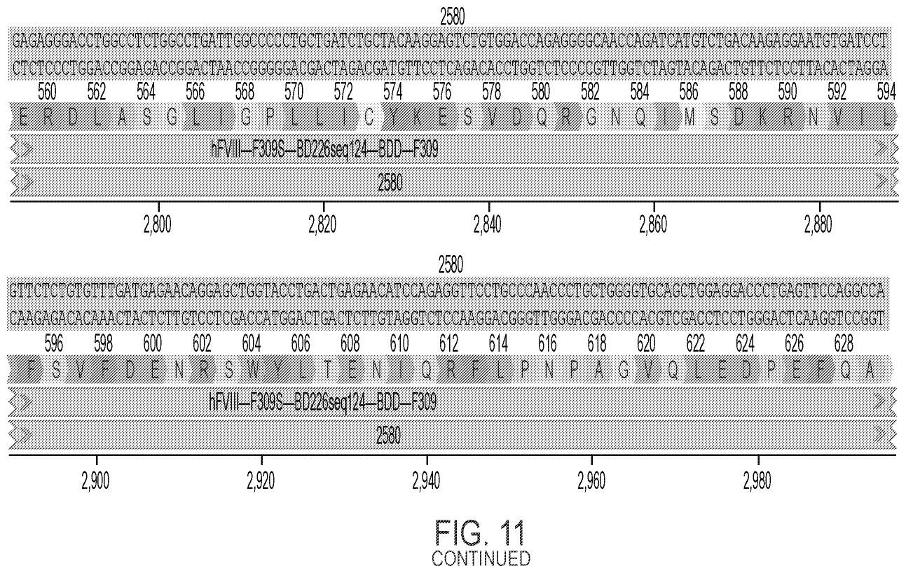

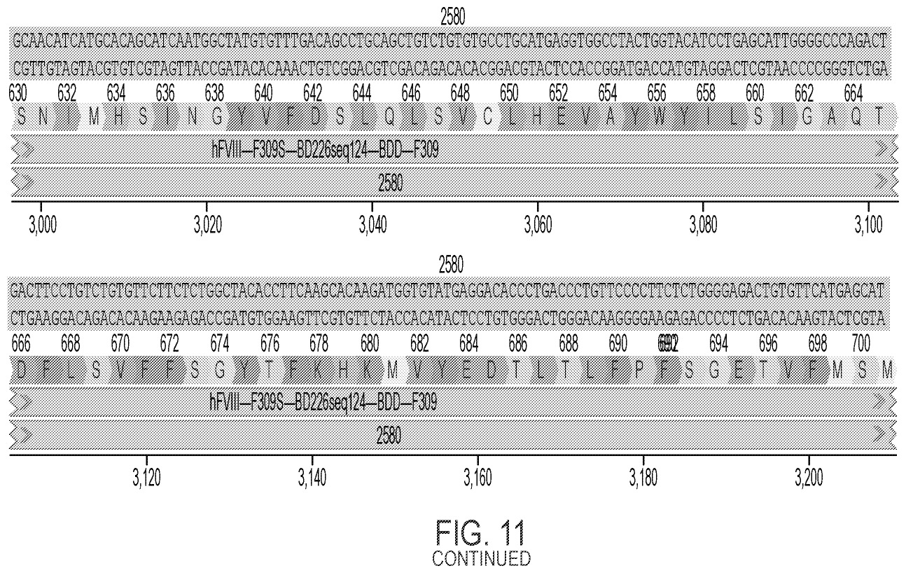

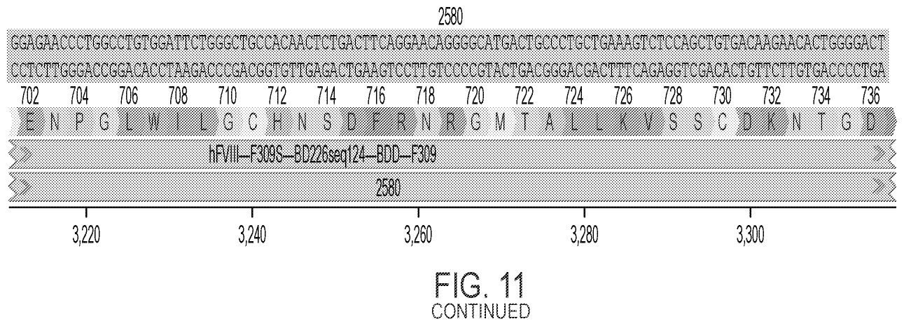

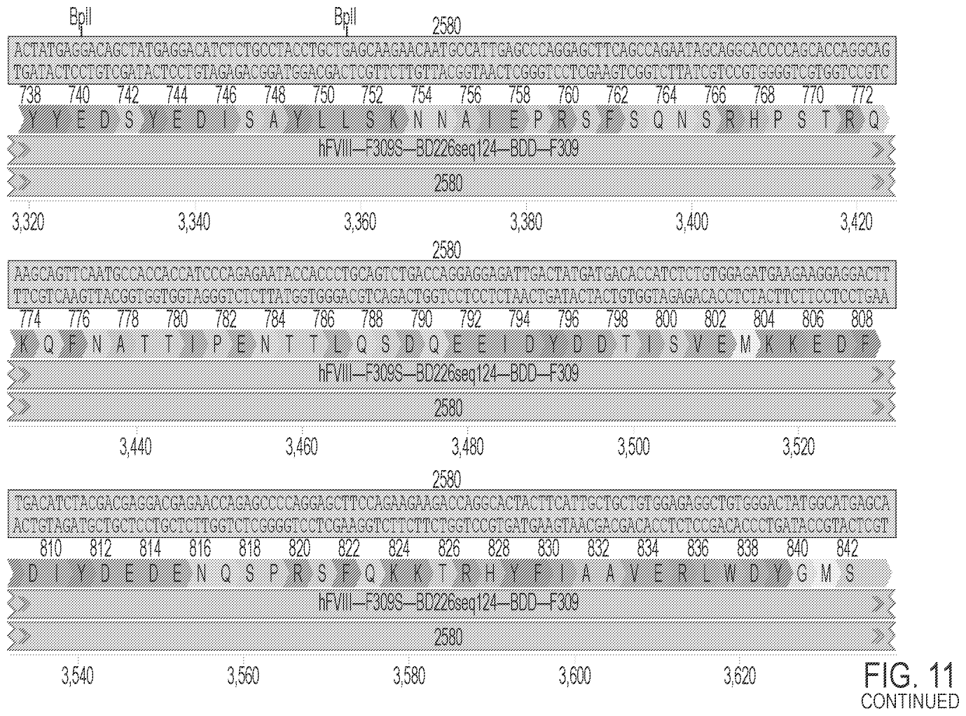

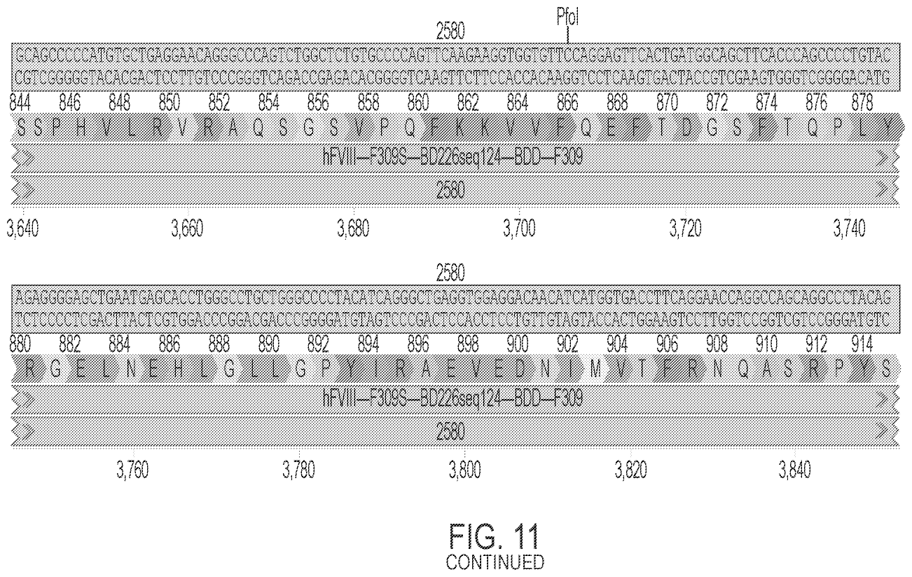

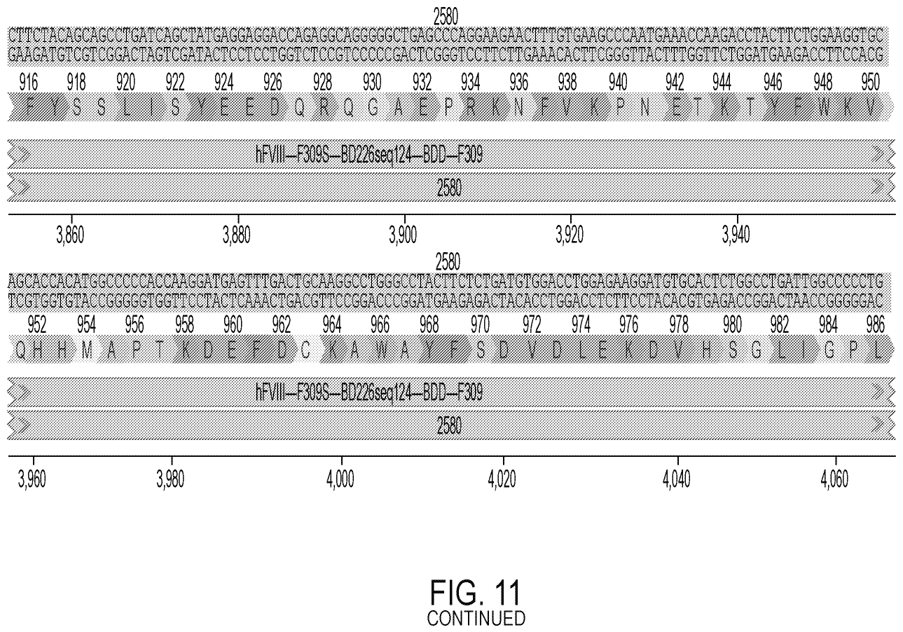

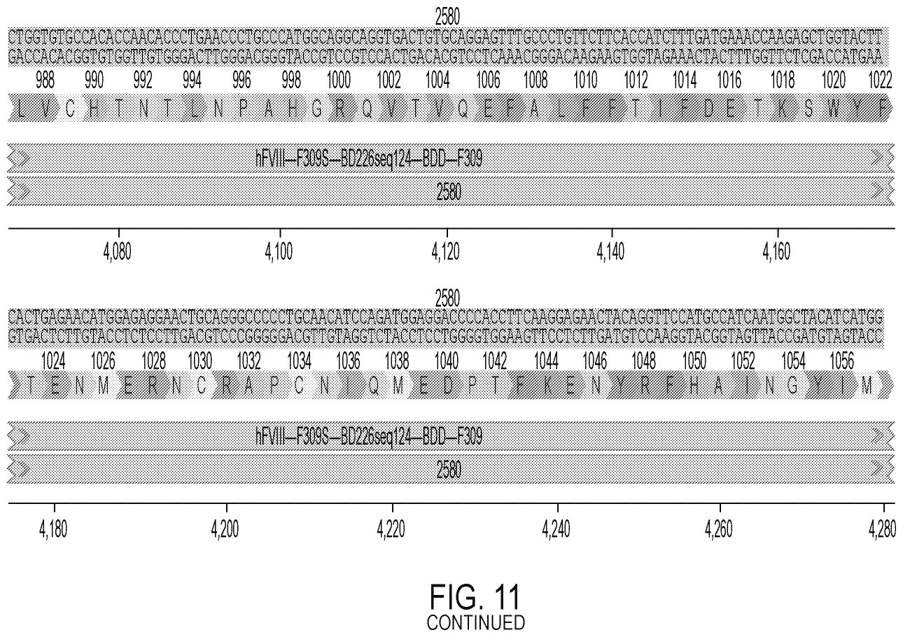

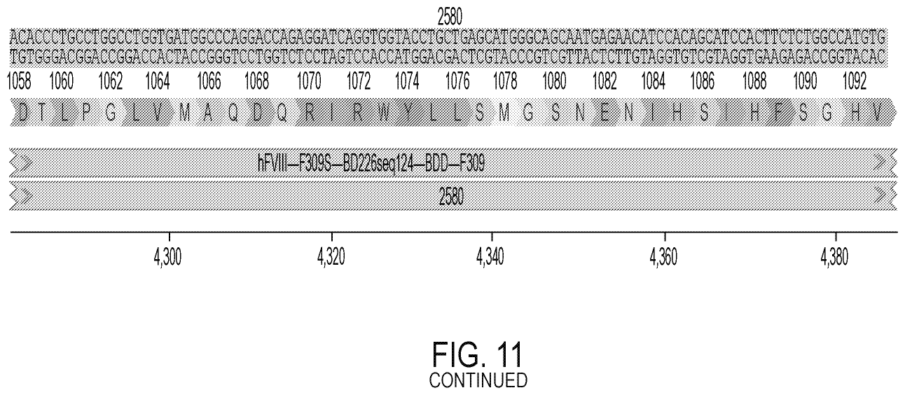

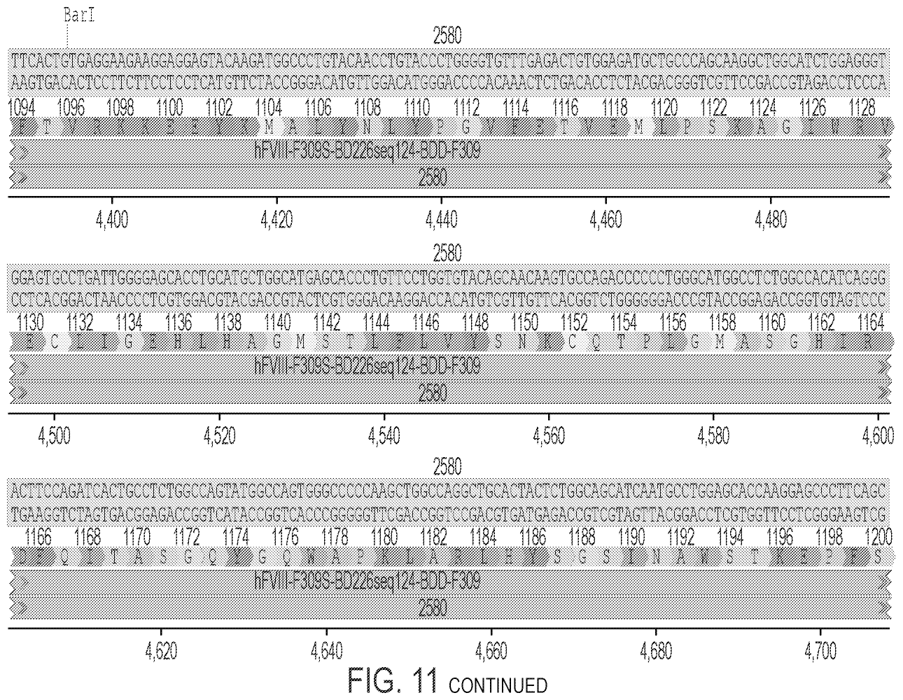

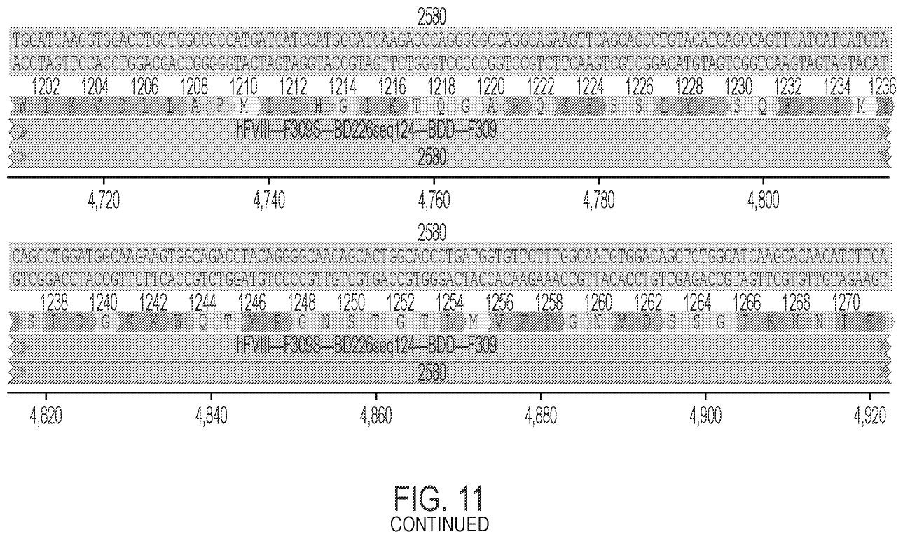

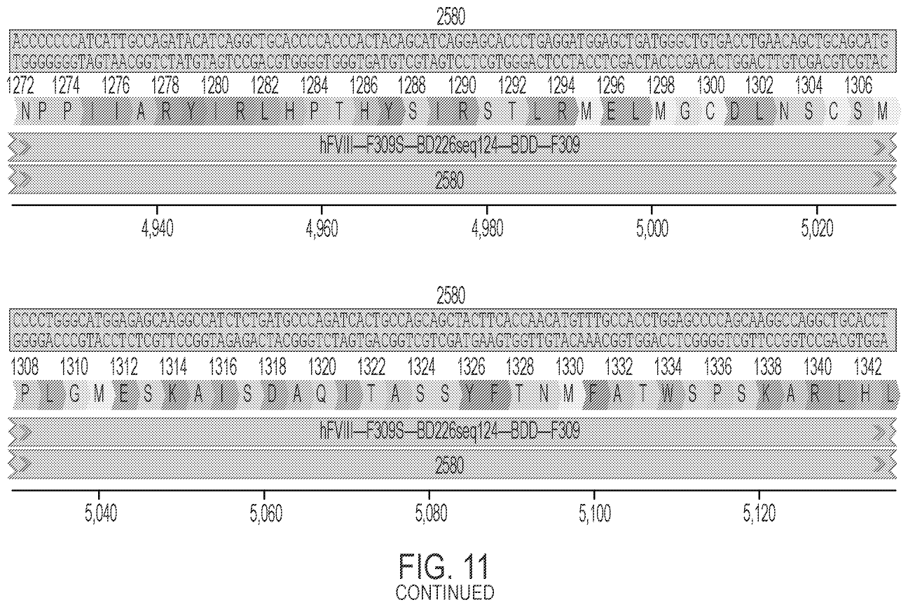

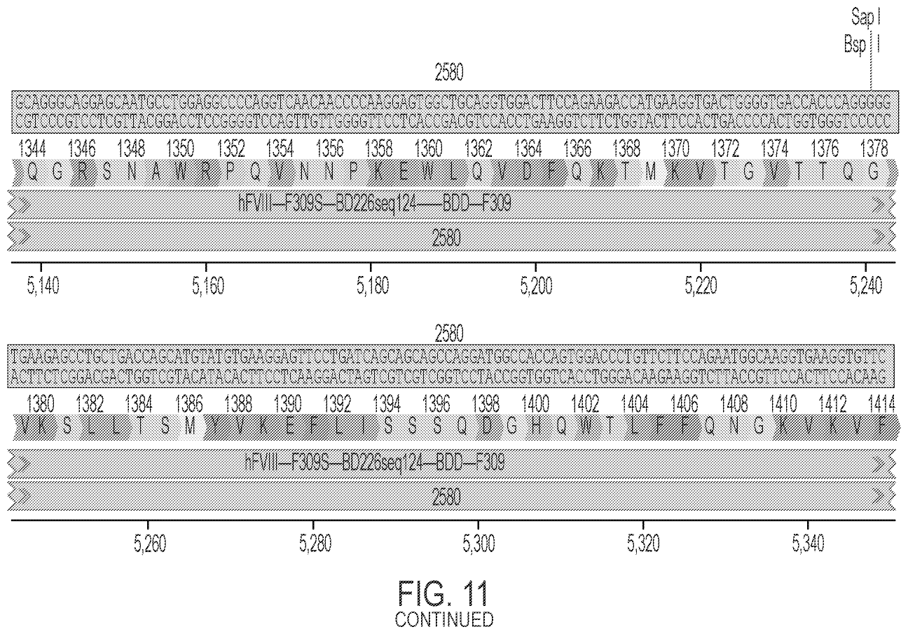

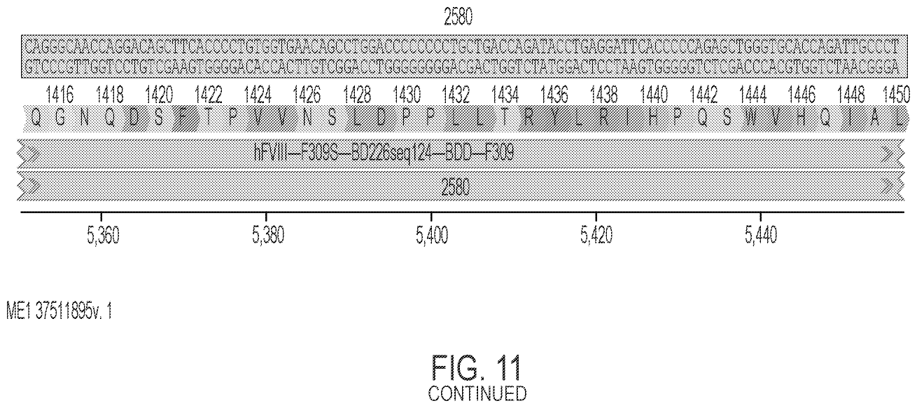

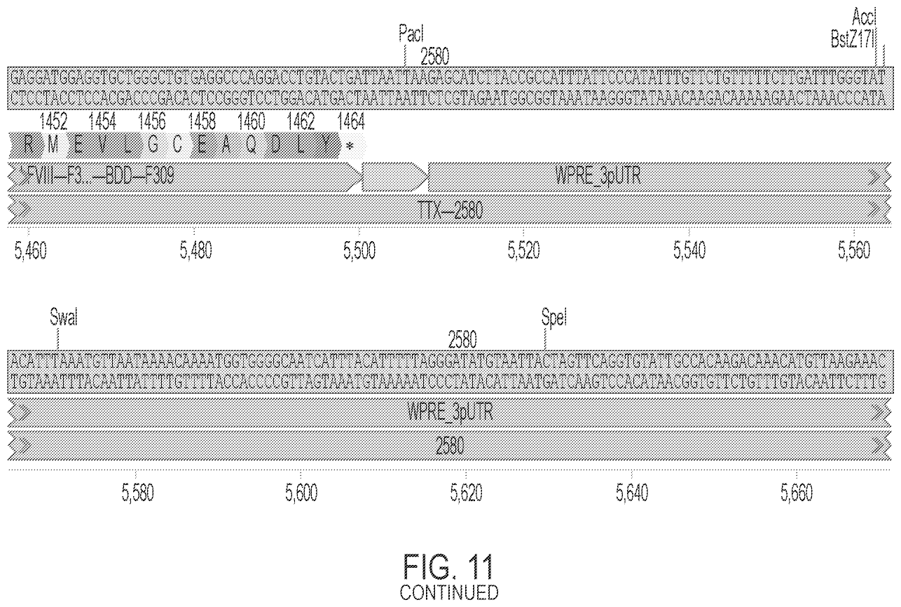

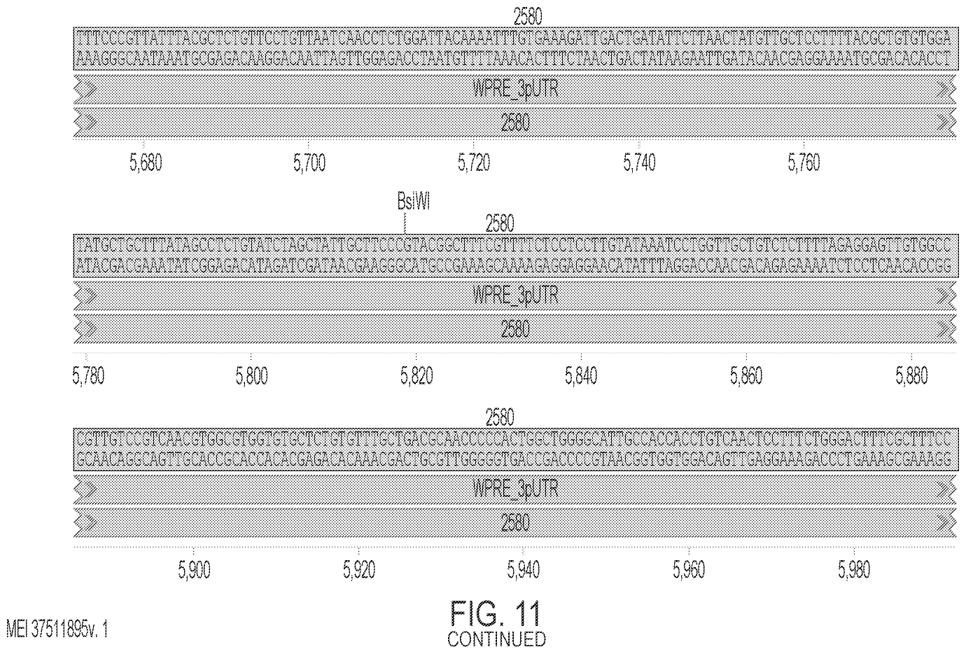

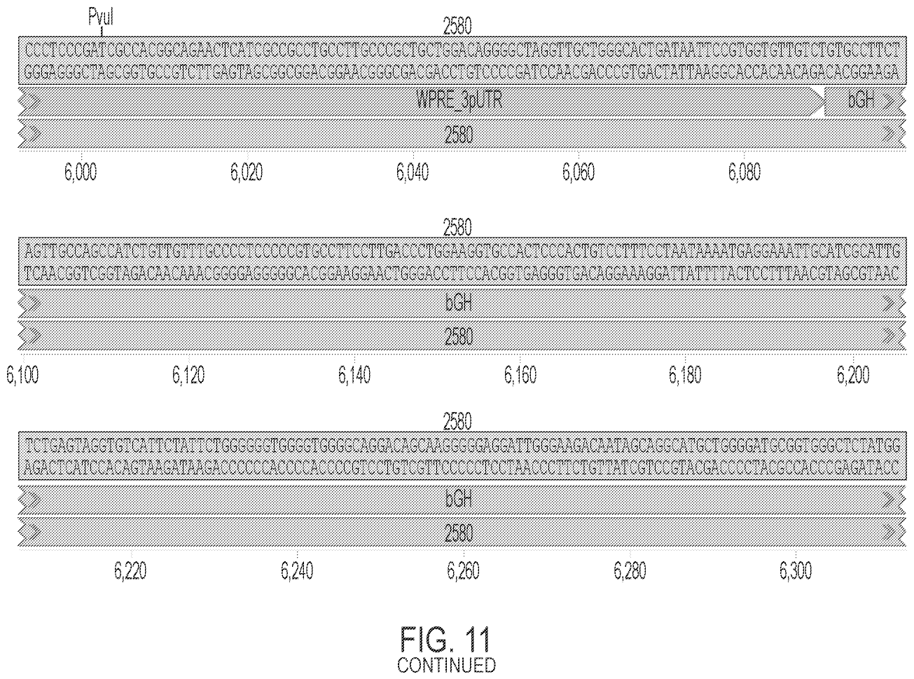

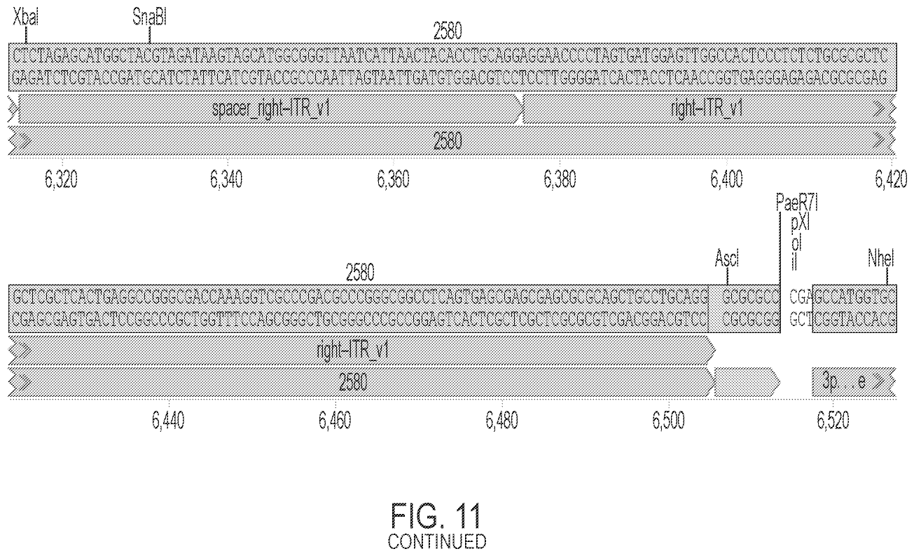

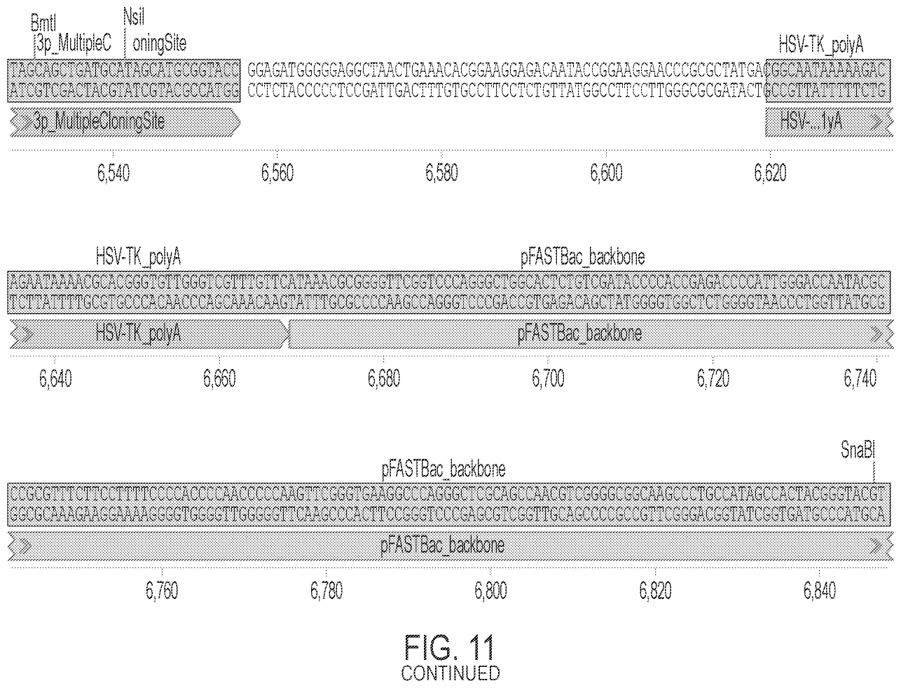

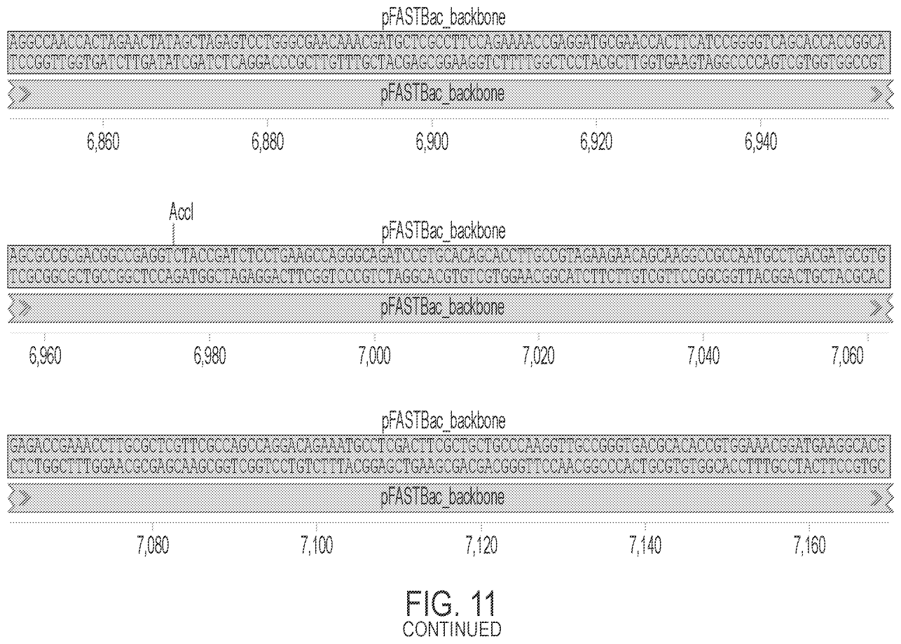

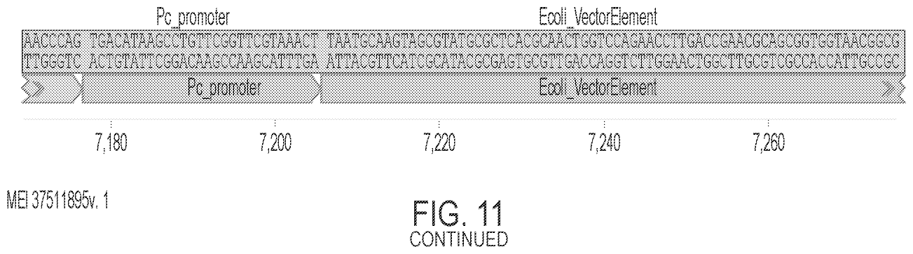

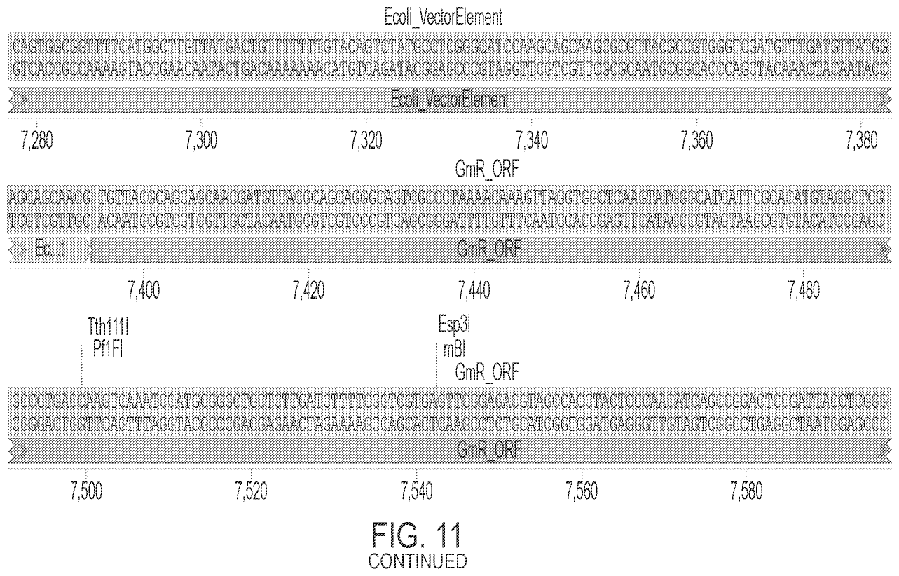

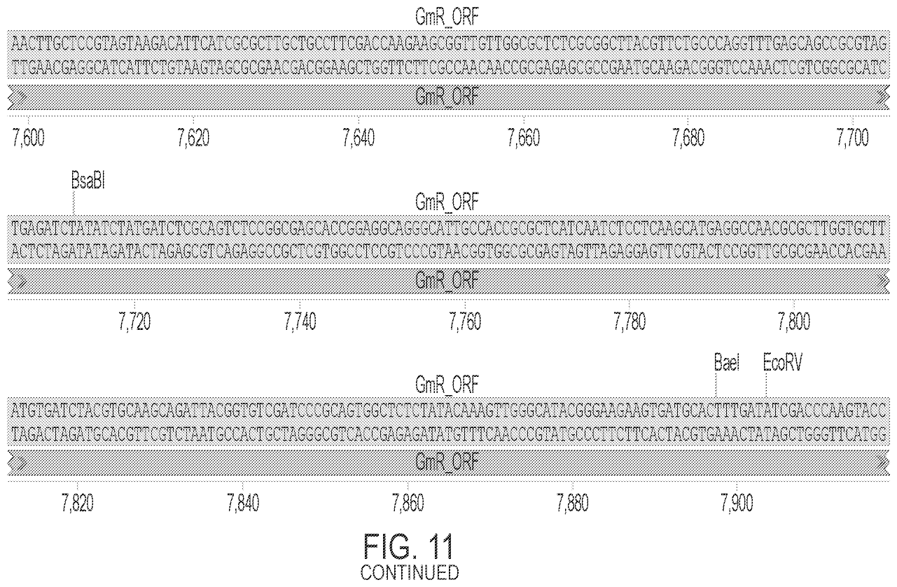

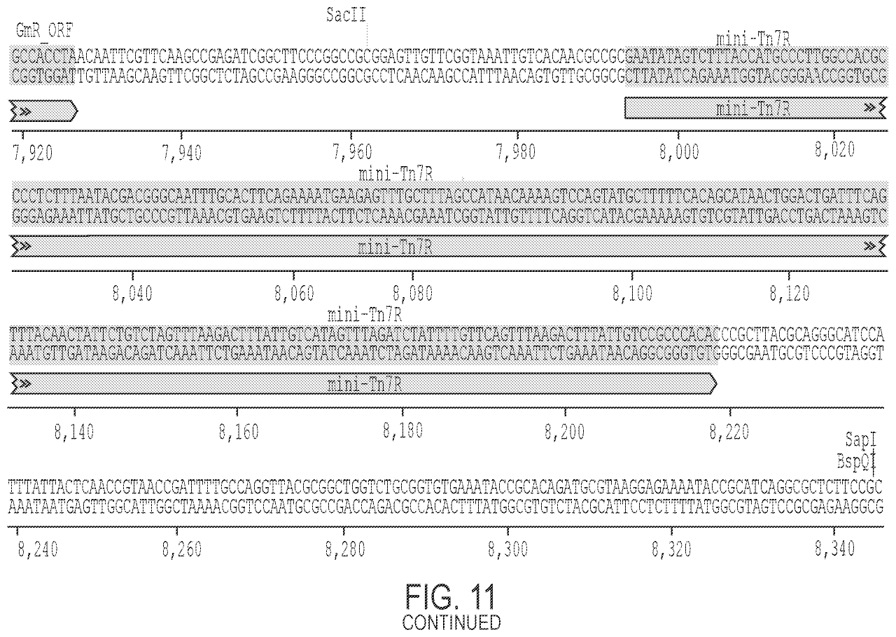

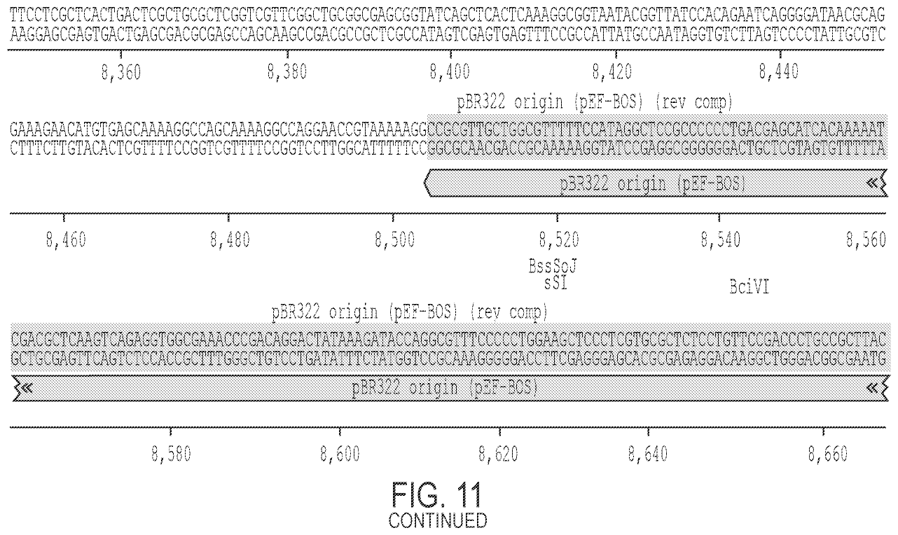

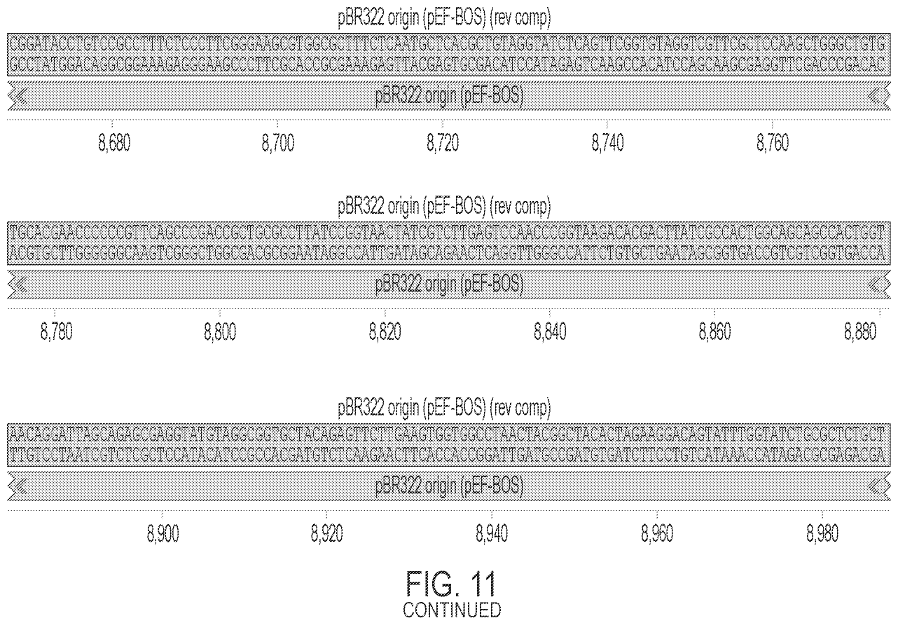

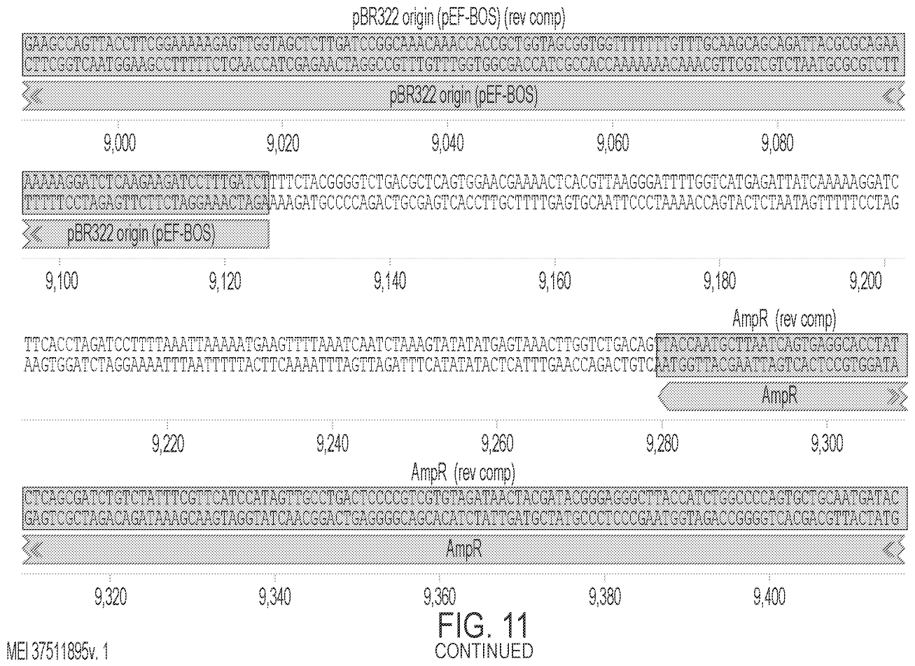

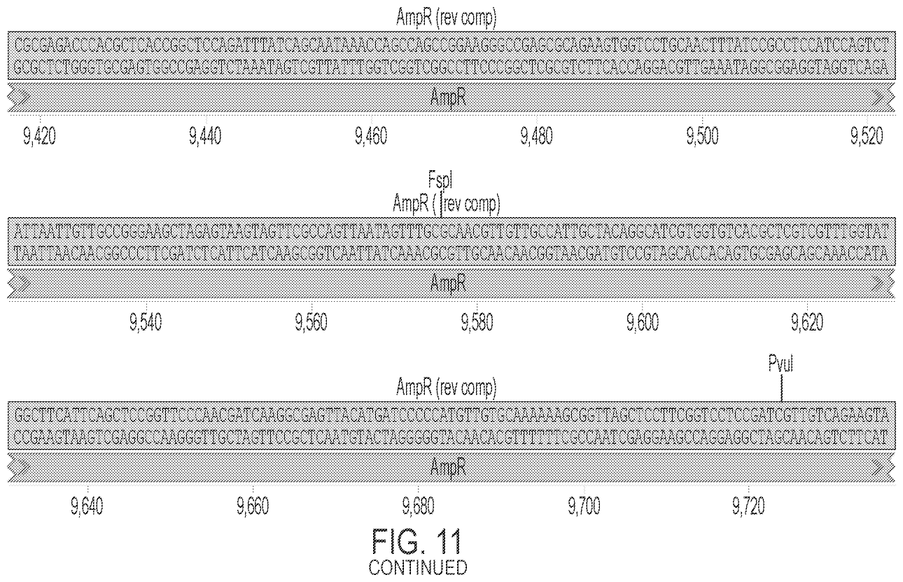

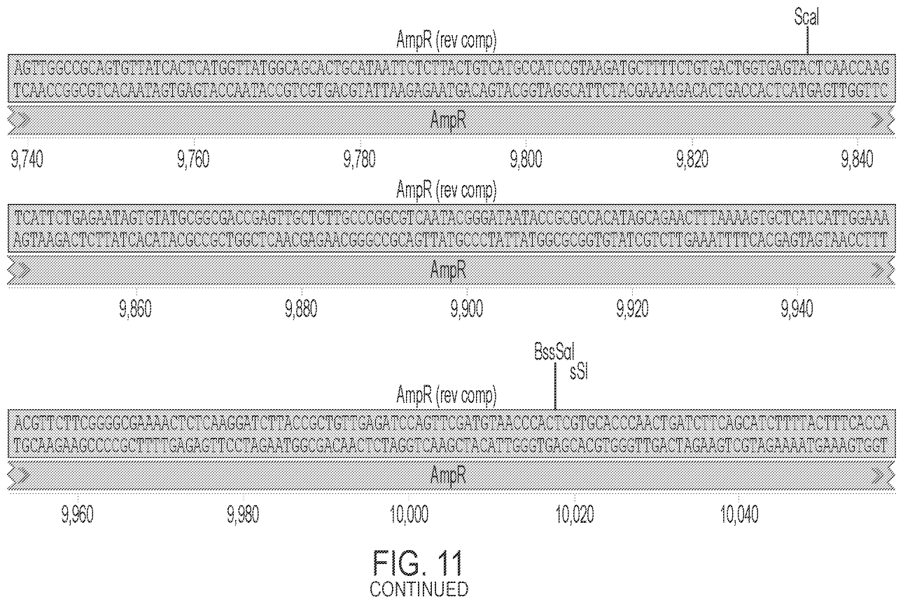

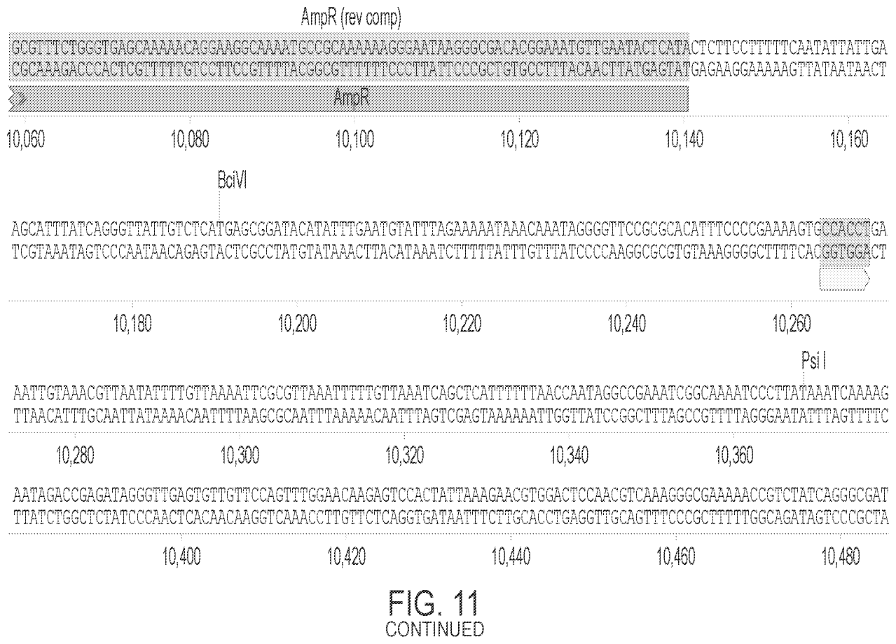

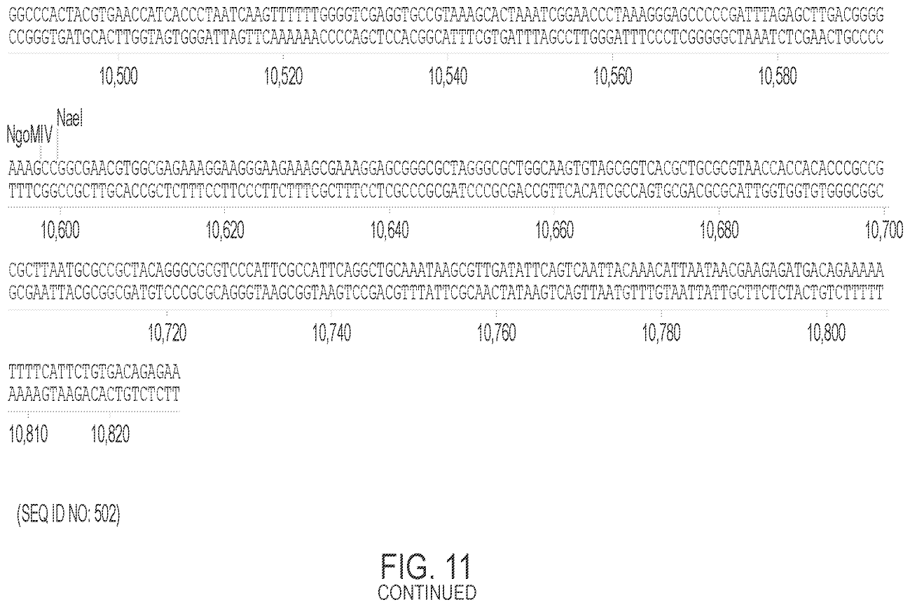

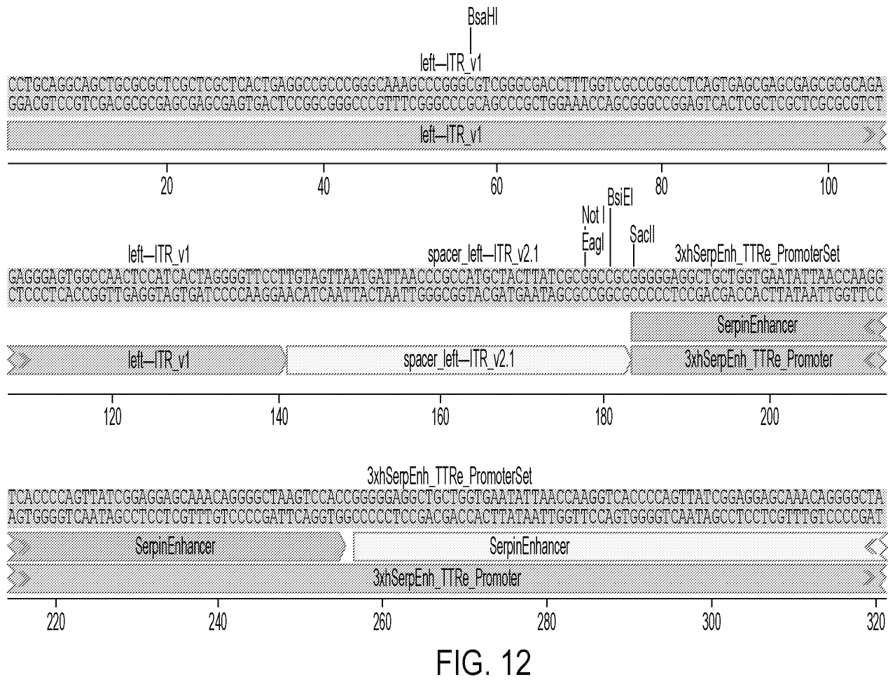

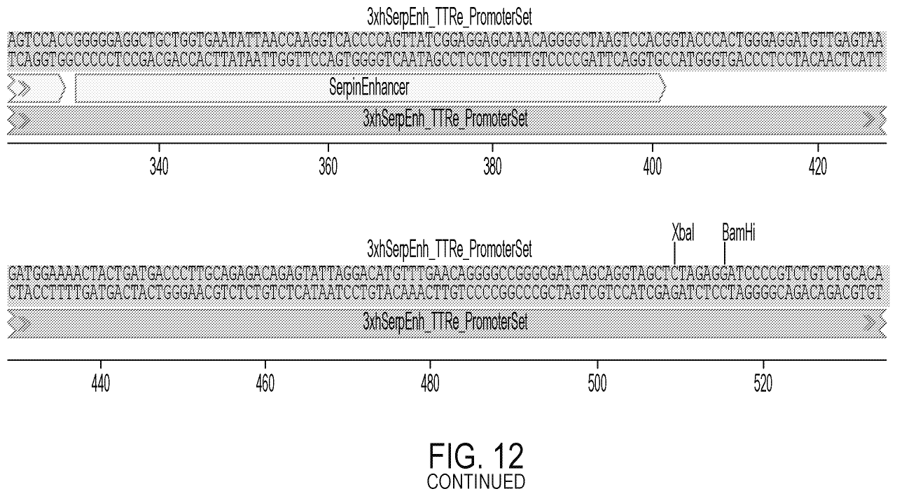

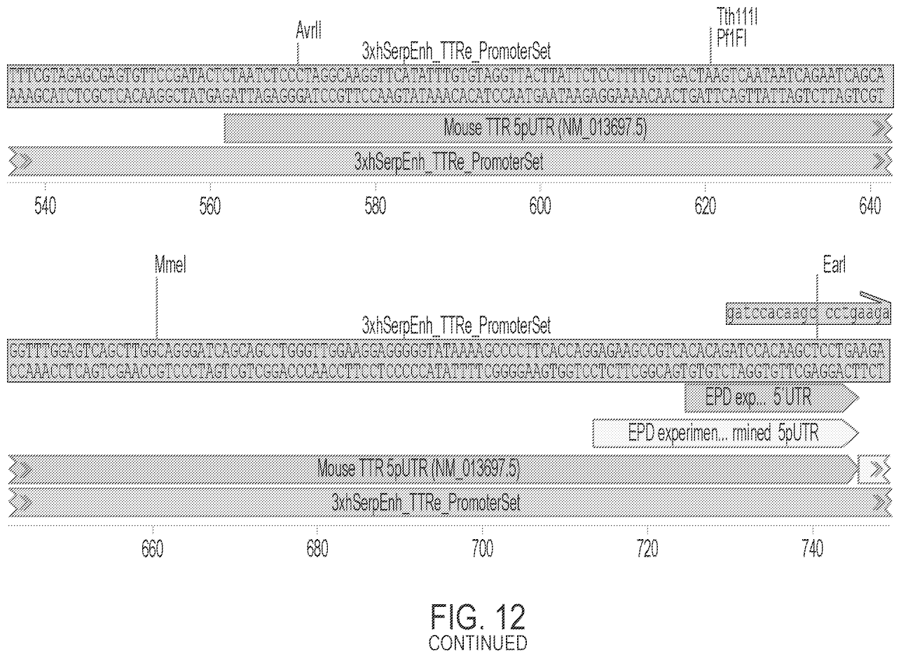

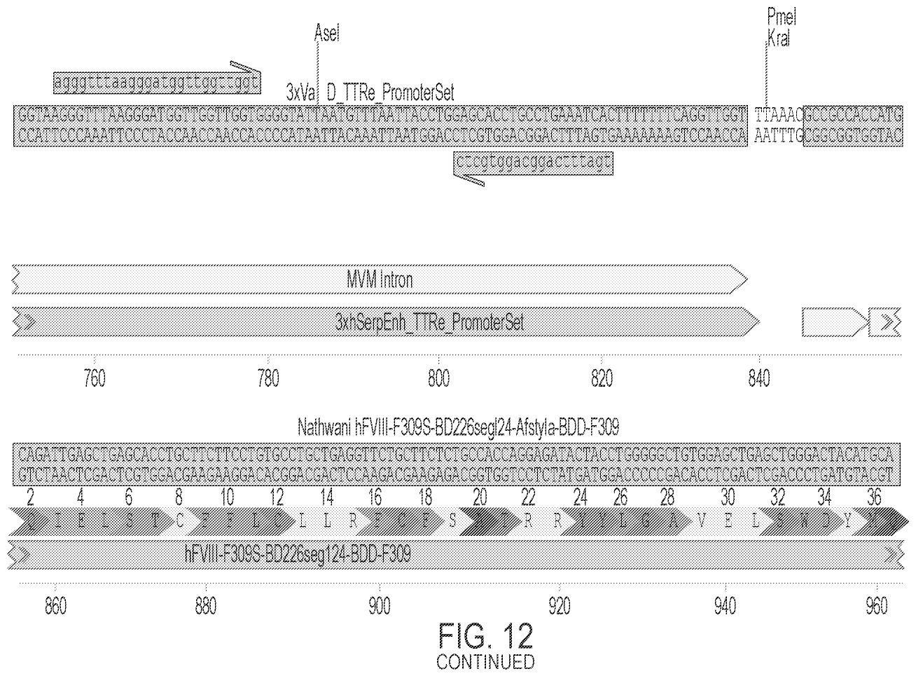

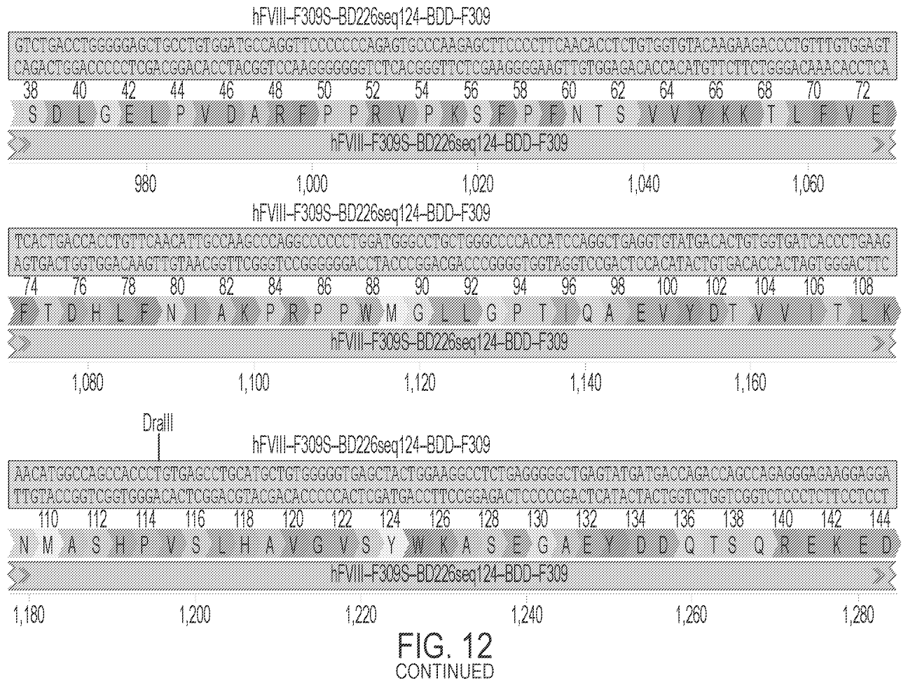

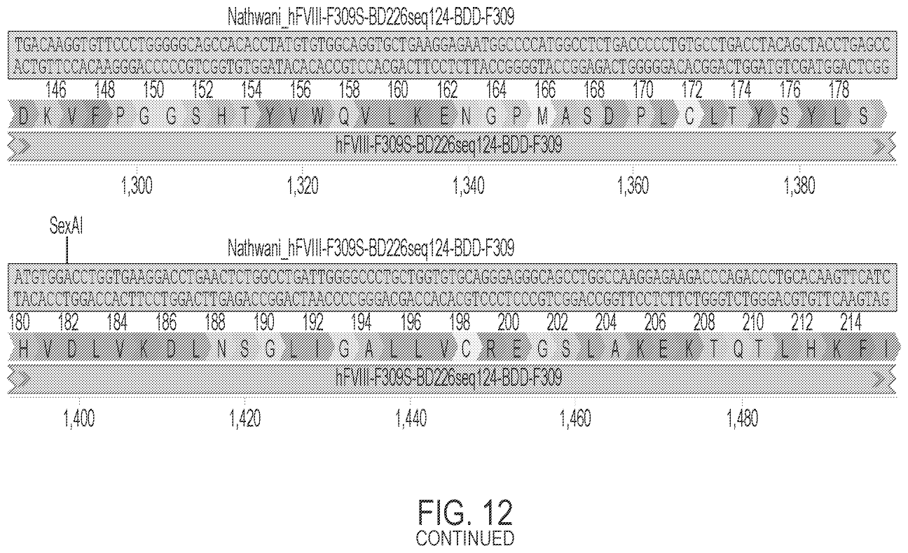

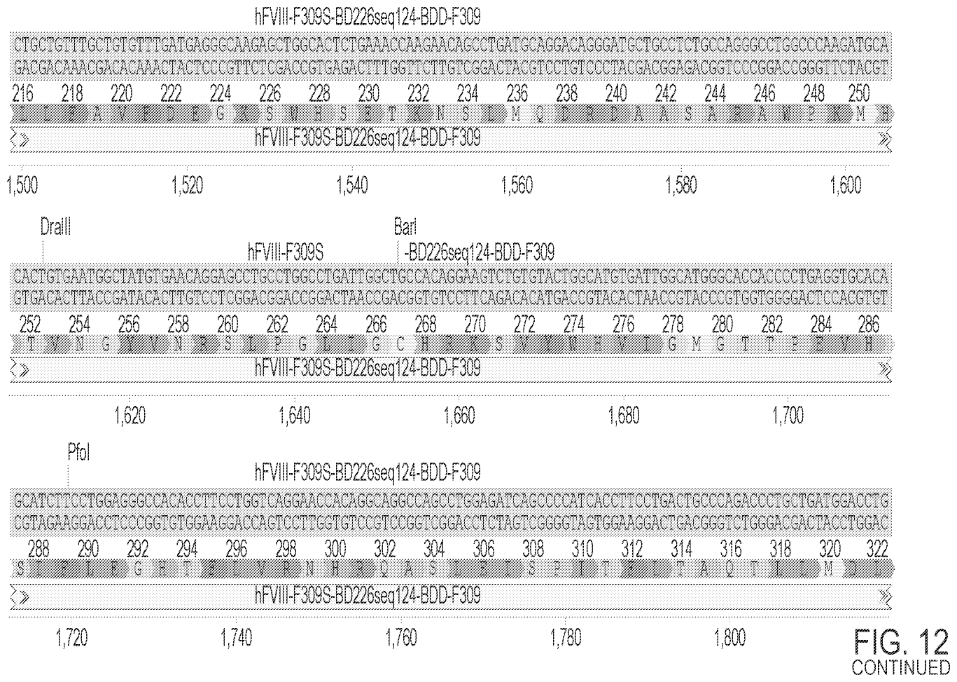

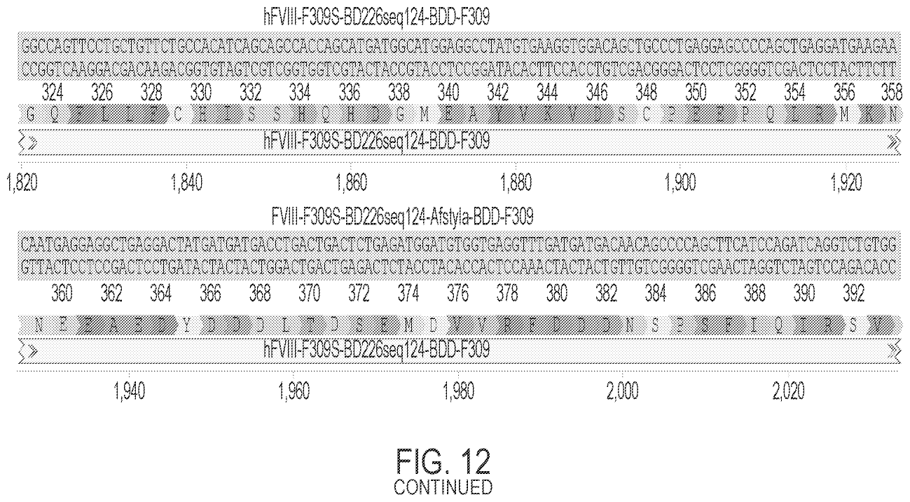

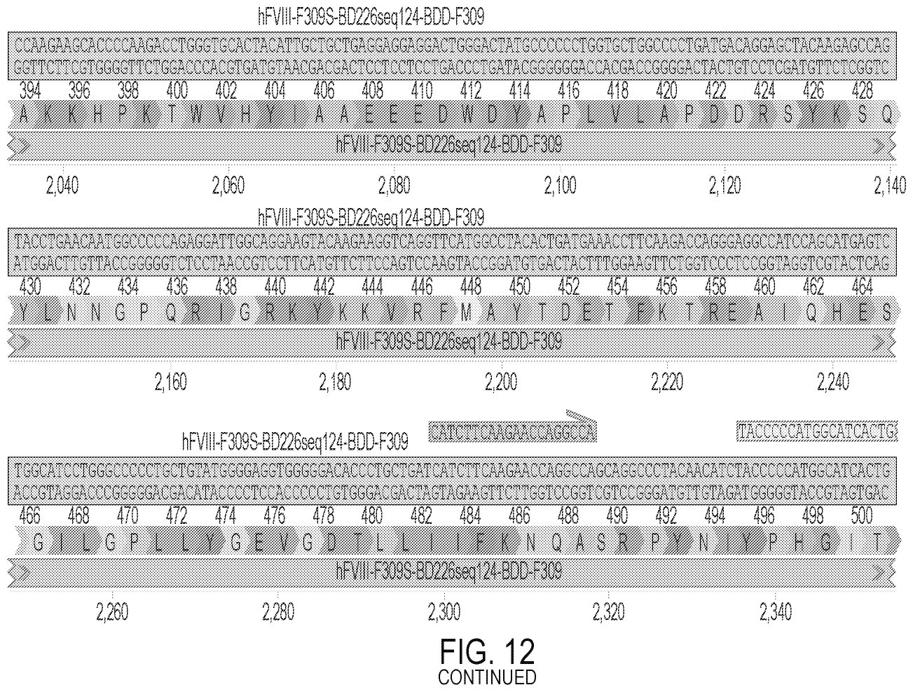

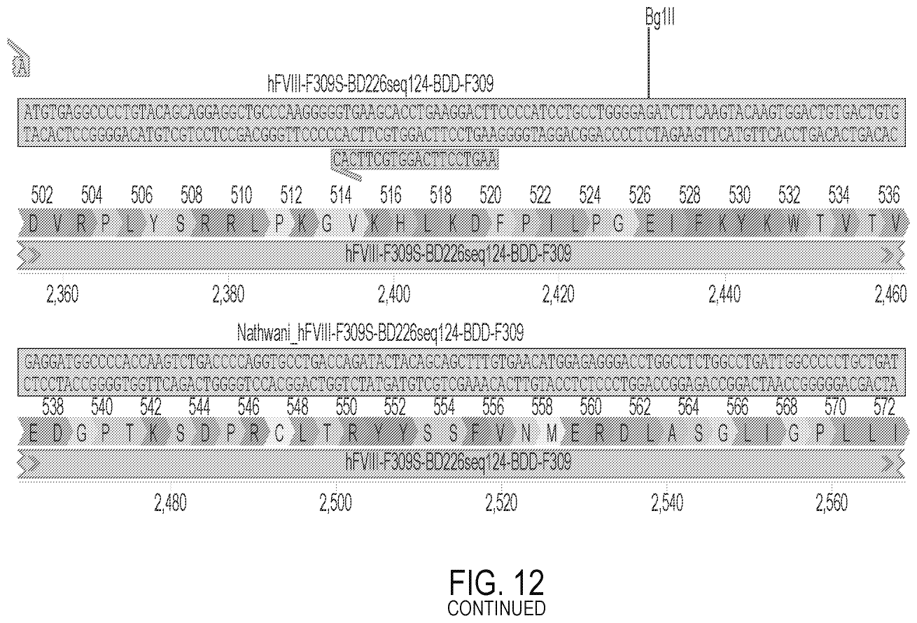

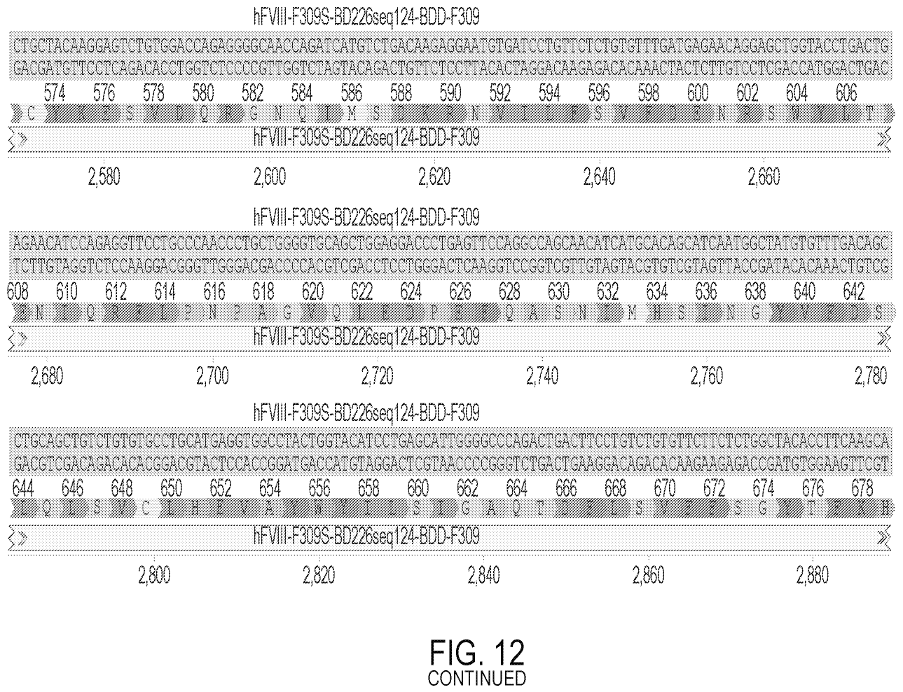

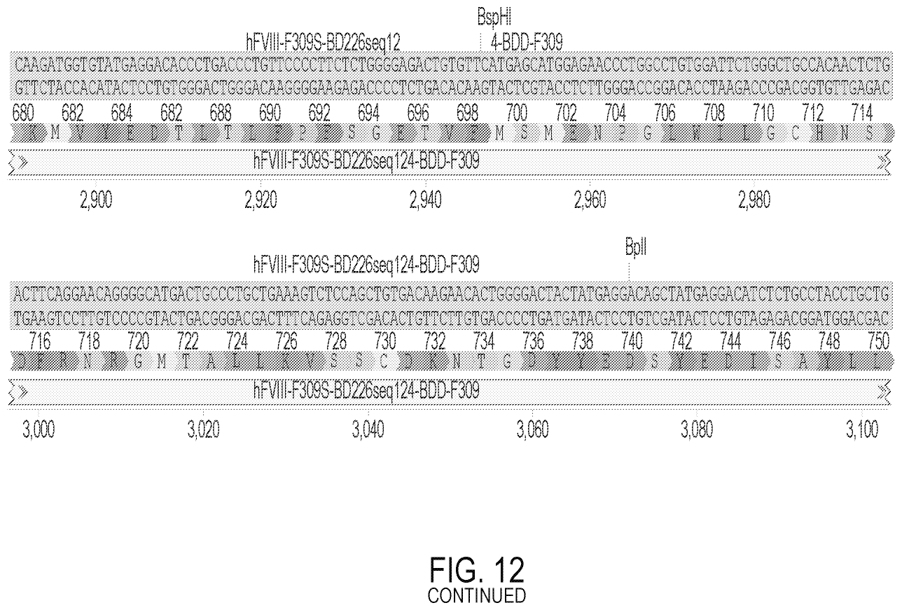

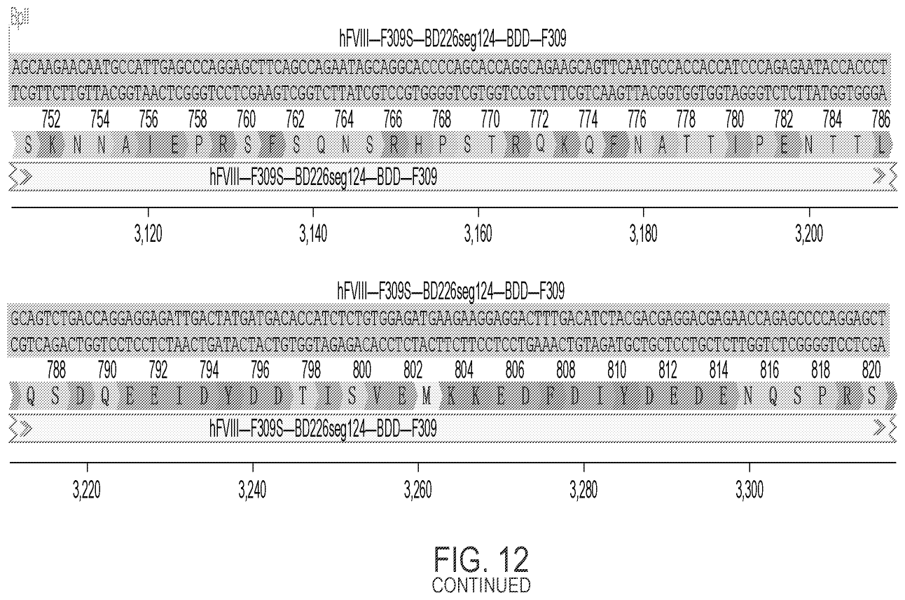

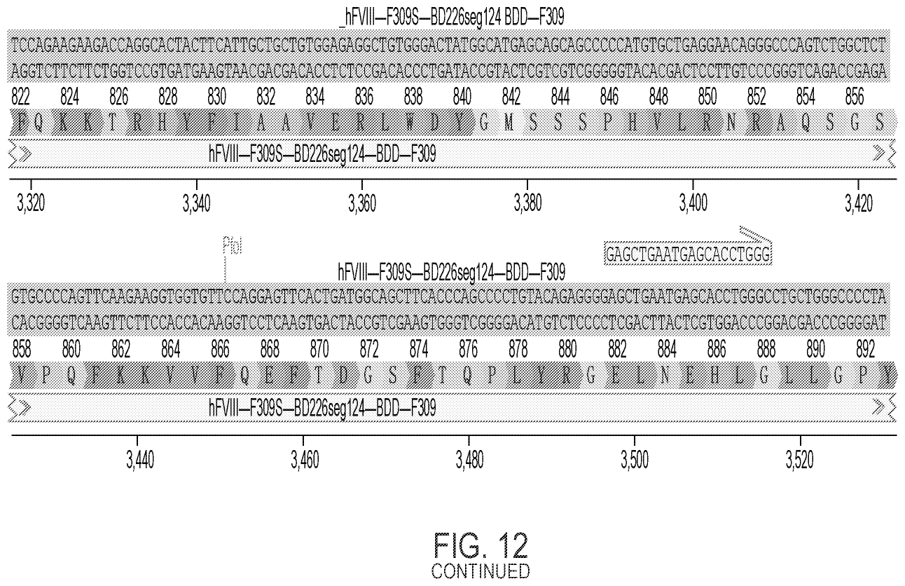

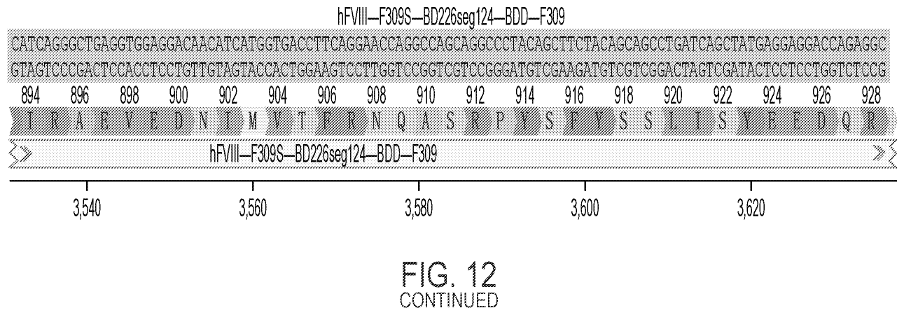

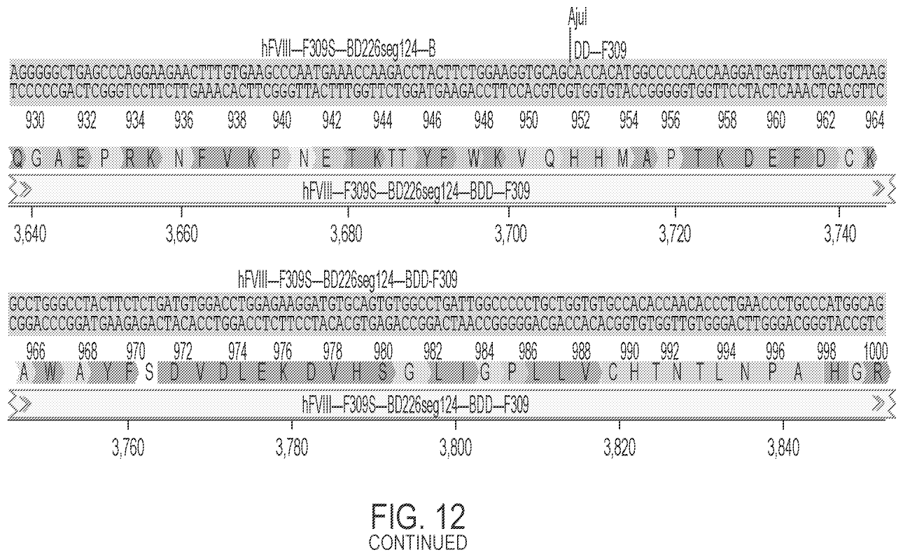

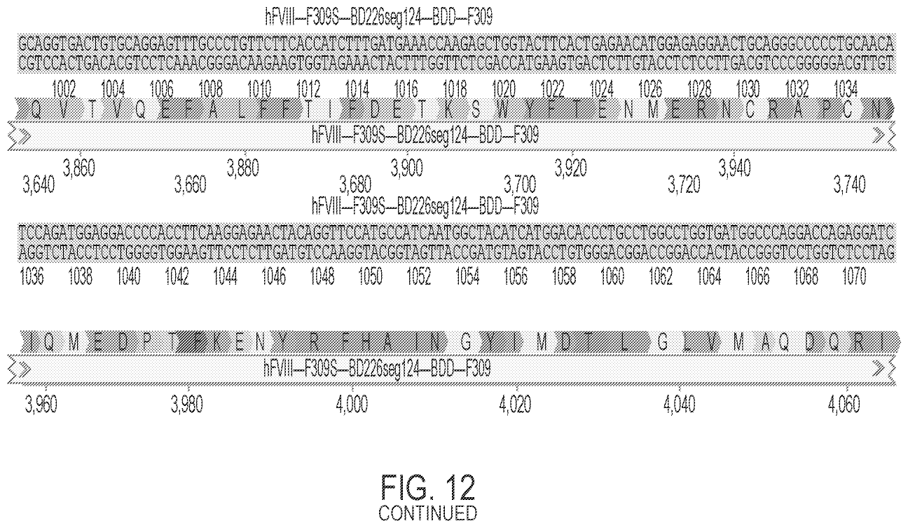

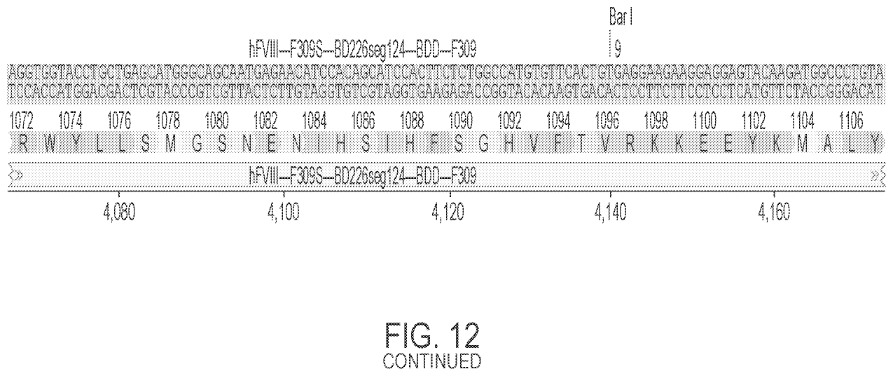

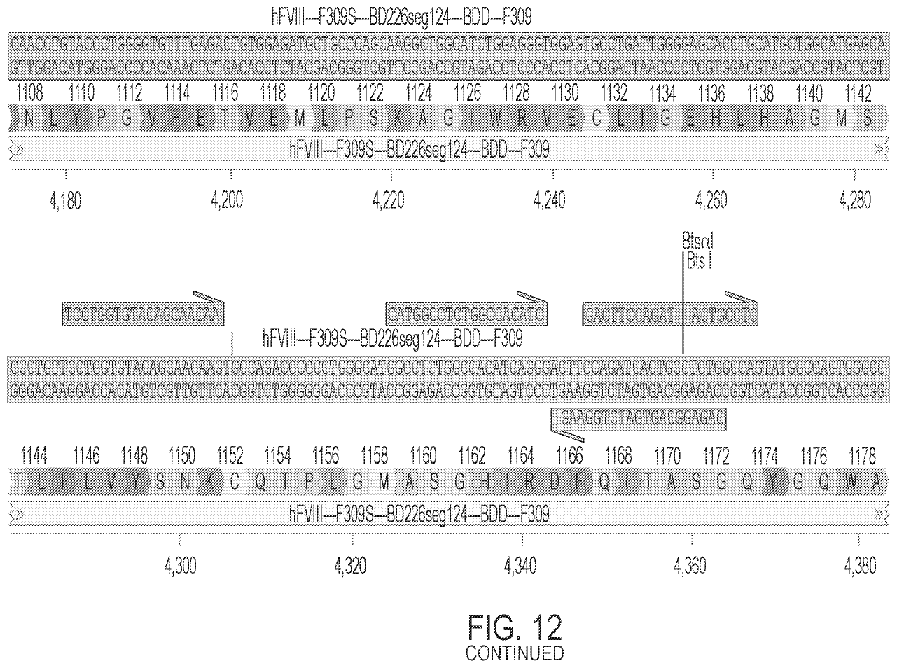

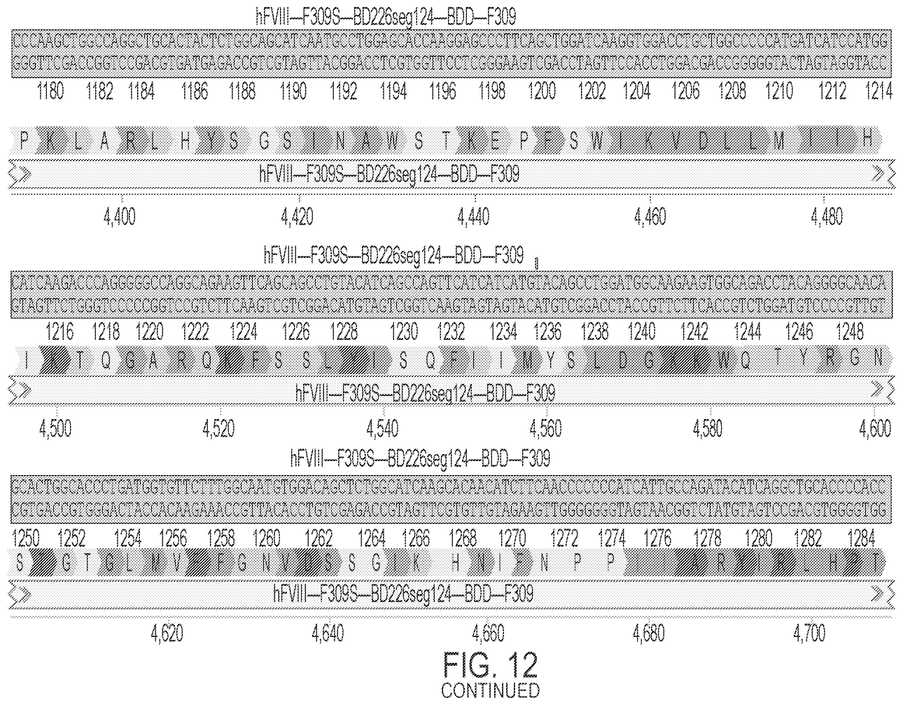

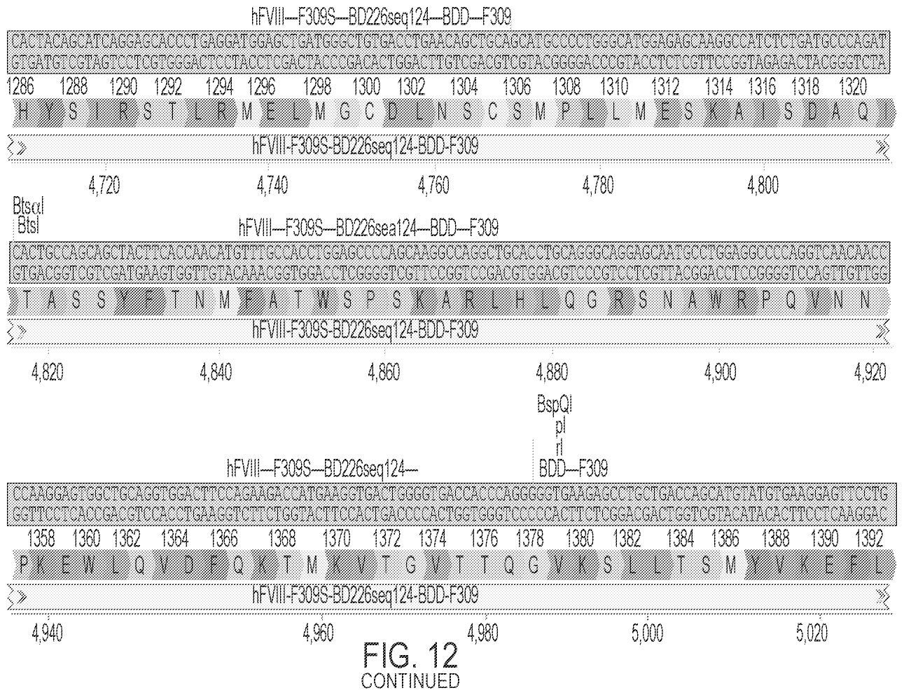

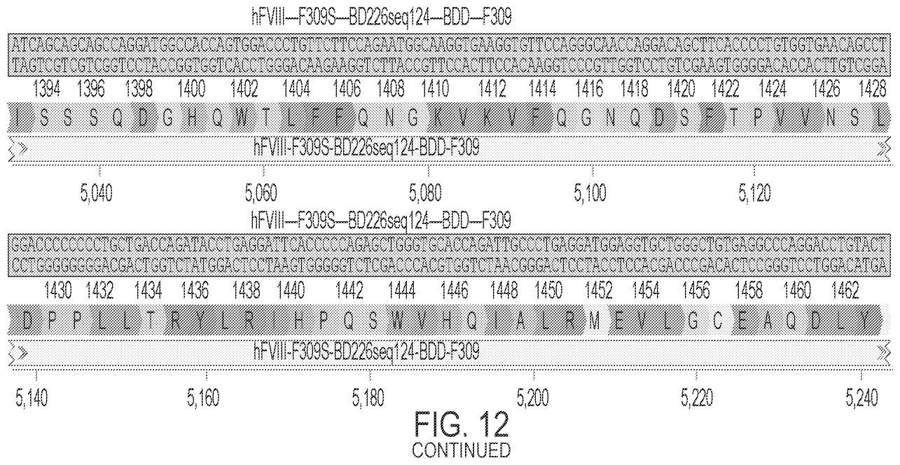

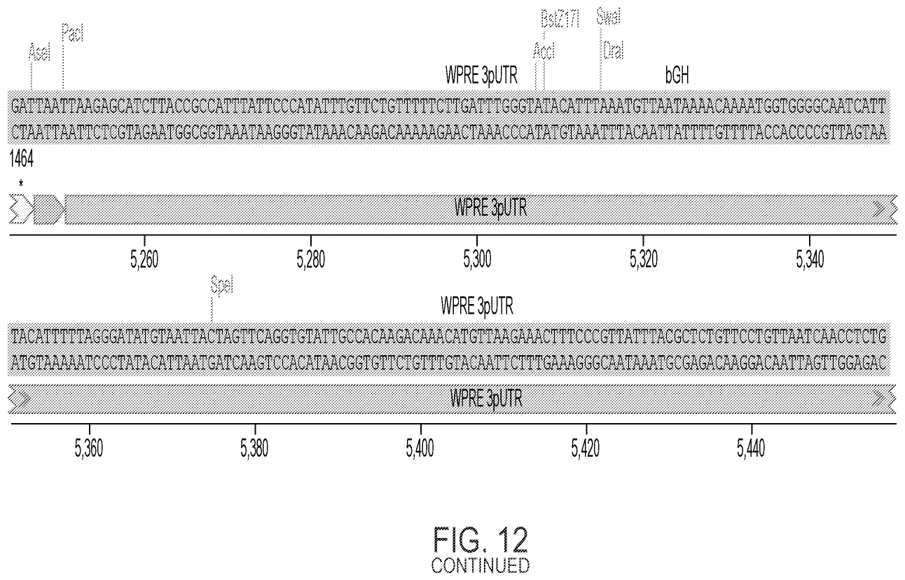

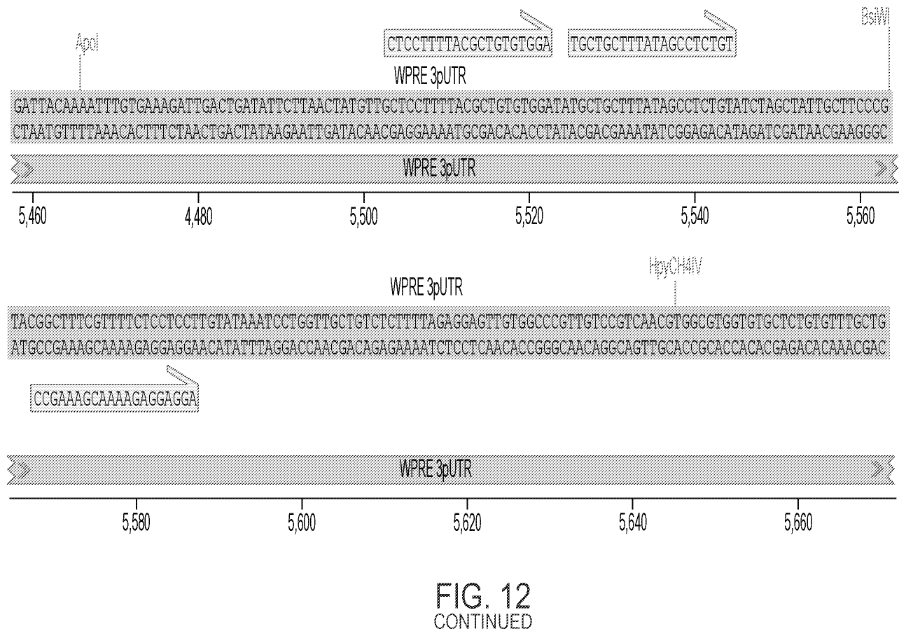

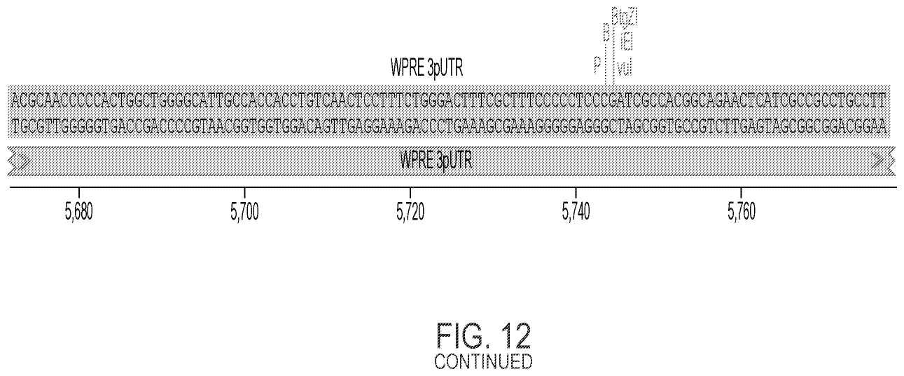

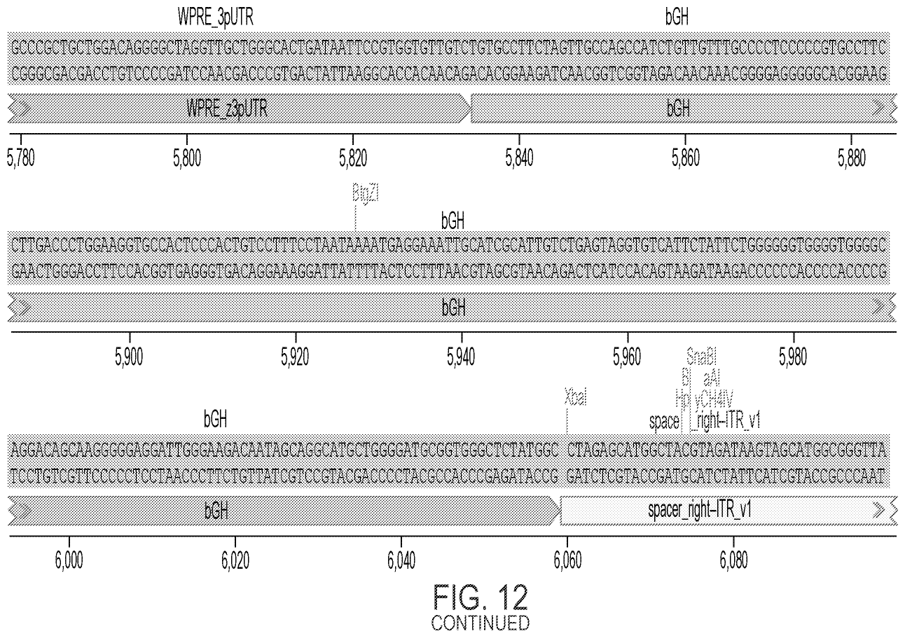

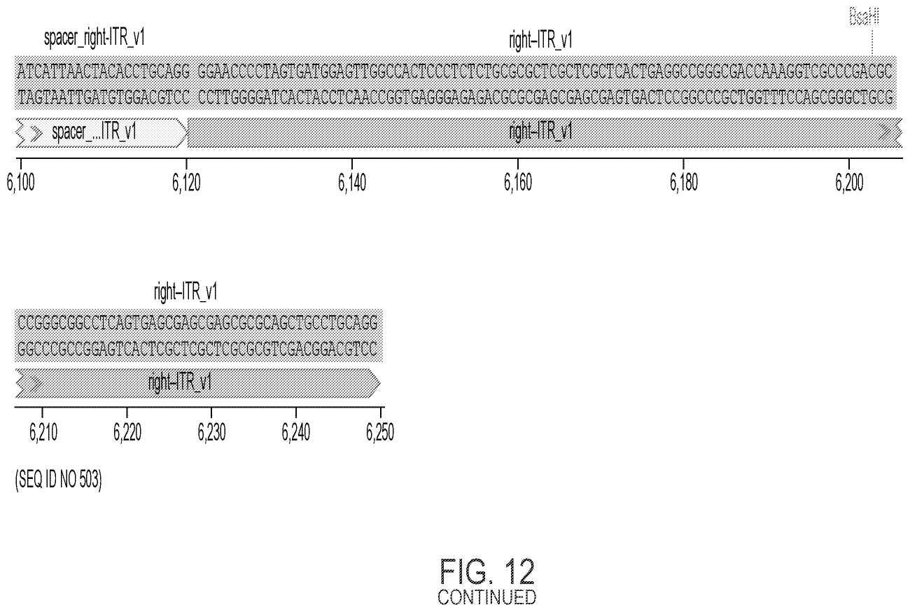

In some embodiments, the ceDNA vector comprises a codon optimized FVIII sequence. In some embodiments, the ceDNA vector comprises a codon optimized FVIII sequence as shown in FIGS. 11 and 12 (hFVIII-F309S-BD226seq124-BDD-F309)In some embodiments, the ceDNA vector comprises an FVIII sequence comprising the nucleic acid sequence as set forth in SEQ ID NO: 143 as shown below: ceDNA 1651 ORF sequence (GE-715; hFVIII-F309S-BD226seq124-BDD-F309)

(SEQ ID NO: 143)
ATGCAGATTGAGCTGAGCACCTGCTTCTTCCTGTGCCTGCTGAGGTTCTGCTTCTCTGCCACCAGGAGA
TACTACCTGGGGGCTGTGGAGCTGAGCTGGGACTACATGCAGTCTGACCTGGGGGAGCTGCCTGTGGAT
GCCAGGTTCCCCCCCAGAGTGCCCAAGAGCTTCCCCTTCAACACCTCTGTGGTGTACAAGAAGACCCTG
TTTGTGGAGTTCACTGACCACCTGTTCAACATTGCCAAGCCCAGGCCCCCCTGGATGGGCCTGCTGGGC
CCCACCATCCAGGCTGAGGTGTATGACACTGTGGTGATCACCCTGAAGAACATGGCCAGCCACCCTGTG
AGCCTGCATGCTGTGGGGGTGAGCTACTGGAAGGCCTCTGAGGGGGCTGAGTATGATGACCAGACCAGC
CAGAGGGAGAAGGAGGATGACAAGGTGTTCCCTGGGGGCAGCCACACCTATGTGTGGCAGGTGCTGAAG
GAGAATGGCCCCATGGCCTCTGACCCCCTGTGCCTGACCTACAGCTACCTGAGCCATGTGGACCTGGTG
AAGGACCTGAACTCTGGCCTGATTGGGGCCCTGCTGGTGTGCAGGGAGGGCAGCCTGGCCAAGGAGAAG
ACCCAGACCCTGCACAAGTTCATCCTGCTGTTTGCTGTGTTTGATGAGGGCAAGAGCTGGCACTCTGAA
ACCAAGAACAGCCTGATGCAGGACAGGGATGCTGCCTCTGCCAGGGCCTGGCCCAAGATGCACACTGTG
AATGGCTATGTGAACAGGAGCCTGCCTGGCCTGATTGGCTGCCACAGGAAGTCTGTGTACTGGCATGTG
ATTGGCATGGGCACCACCCCTGAGGTGCACAGCATCTTCCTGGAGGGCCACACCTTCCTGGTCAGGAAC
CACAGGCAGGCCAGCCTGGAGATCAGCCCCATCACCTTCCTGACTGCCCAGACCCTGCTGATGGACCTG
GGCCAGTTCCTGCTGTTCTGCCACATCAGCAGCCACCAGCATGATGGCATGGAGGCCTATGTGAAGGTG
GACAGCTGCCCTGAGGAGCCCCAGCTGAGGATGAAGAACAATGAGGAGGCTGAGGACTATGATGATGAC
CTGACTGACTCTGAGATGGATGTGGTGAGGTTTGATGATGACAACAGCCCCAGCTTCATCCAGATCAGG
TCTGTGGCCAAGAAGCACCCCAAGACCTGGGTGCACTACATTGCTGCTGAGGAGGAGGACTGGGACTAT
GCCCCCCTGGTGCTGGCCCCTGATGACAGGAGCTACAAGAGCCAGTACCTGAACAATGGCCCCCAGAGG
ATTGGCAGGAAGTACAAGAAGGTCAGGTTCATGGCCTACACTGATGAAACCTTCAAGACCAGGGAGGCC
ATCCAGCATGAGTCTGGCATCCTGGGCCCCCTGCTGTATGGGGAGGTGGGGGACACCCTGCTGATCATC
TTCAAGAACCAGGCCAGCAGGCCCTACAACATCTACCCCCATGGCATCACTGATGTGAGGCCCCTGTAC
AGCAGGAGGCTGCCCAAGGGGGTGAAGCACCTGAAGGACTTCCCCATCCTGCCTGGGGAGATCTTCAAG
TACAAGTGGACTGTGACTGTGGAGGATGGCCCCACCAAGTCTGACCCCAGGTGCCTGACCAGATACTAC
AGCAGCTTTGTGAACATGGAGAGGGACCTGGCCTCTGGCCTGATTGGCCCCCTGCTGATCTGCTACAAG
GAGTCTGTGGACCAGAGGGGCAACCAGATCATGTCTGACAAGAGGAATGTGATCCTGTTCTCTGTGTTT
GATGAGAACAGGAGCTGGTACCTGACTGAGAACATCCAGAGGTTCCTGCCCAACCCTGCTGGGGTGCAG
CTGGAGGACCCTGAGTTCCAGGCCAGCAACATCATGCACAGCATCAATGGCTATGTGTTTGACAGCCTG
CAGCTGTCTGTGTGCCTGCATGAGGTGGCCTACTGGTACATCCTGAGCATTGGGGCCCAGACTGACTTC
CTGTCTGTGTTCTTCTCTGGCTACACCTTCAAGCACAAGATGGTGTATGAGGACACCCTGACCCTGTTC
CCCTTCTCTGGGGAGACTGTGTTCATGAGCATGGAGAACCCTGGCCTGTGGATTCTGGGCTGCCACAAC
TCTGACTTCAGGAACAGGGGCATGACTGCCCTGCTGAAAGTCTCCAGCTGTGACAAGAACACTGGGGAC
TACTATGAGGACAGCTATGAGGACATCTCTGCCTACCTGCTGAGCAAGAACAATGCCATTGAGCCCAGG
AGCTTCAGCCAGAATAGCAGGCACCCCAGCACCAGGCAGAAGCAGTTCAATGCCACCACCATCCCAGAG
AATACCACCCTGCAGTCTGACCAGGAGGAGATTGACTATGATGACACCATCTCTGTGGAGATGAAGAAG
GAGGACTTTGACATCTACGACGAGGACGAGAACCAGAGCCCCAGGAGCTTCCAGAAGAAGACCAGGCAC
TACTTCATTGCTGCTGTGGAGAGGCTGTGGGACTATGGCATGAGCAGCAGCCCCCATGTGCTGAGGAAC
AGGGCCCAGTCTGGCTCTGTGCCCCAGTTCAAGAAGGTGGTGTTCCAGGAGTTCACTGATGGCAGCTTC
ACCCAGCCCCTGTACAGAGGGGAGCTGAATGAGCACCTGGGCCTGCTGGGCCCCTACATCAGGGCTGAG
GTGGAGGACAACATCATGGTGACCTTCAGGAACCAGGCCAGCAGGCCCTACAGCTTCTACAGCAGCCTG
ATCAGCTATGAGGAGGACCAGAGGCAGGGGGCTGAGCCCAGGAAGAACTTTGTGAAGCCCAATGAAACC
AAGACCTACTTCTGGAAGGTGCAGCACCACATGGCCCCCACCAAGGATGAGTTTGACTGCAAGGCCTGG
GCCTACTTCTCTGATGTGGACCTGGAGAAGGATGTGCACTCTGGCCTGATTGGCCCCCTGCTGGTGTGC
CACACCAACACCCTGAACCCTGCCCATGGCAGGCAGGTGACTGTGCAGGAGTTTGCCCTGTTCTTCACC
ATCTTTGATGAAACCAAGAGCTGGTACTTCACTGAGAACATGGAGAGGAACTGCAGGGCCCCCTGCAAC
ATCCAGATGGAGGACCCCACCTTCAAGGAGAACTACAGGTTCCATGCCATCAATGGCTACATCATGGAC
ACCCTGCCTGGCCTGGTGATGGCCCAGGACCAGAGGATCAGGTGGTACCTGCTGAGCATGGGCAGCAAT
GAGAACATCCACAGCATCCACTTCTCTGGCCATGTGTTCACTGTGAGGAAGAAGGAGGAGTACAAGATG
GCCCTGTACAACCTGTACCCTGGGGTGTTTGAGACTGTGGAGATGCTGCCCAGCAAGGCTGGCATCTGG
AGGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCAG
TATGGCCAGTGGGCCCCCAAGCTGGCCAGGCTGCACTACTCTGGCAGCATCAATGCCTGGAGCACCAAG
GAGCCCTTCAGCTGGATCAAGGTGGACCTGCTGGCCCCCATGATCATCCATGGCATCAAGACCCAGGGG
GCCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGGC
ATCAAGCACAACATCTTCAACCCCCCCATCATTGCCAGATACATCAGGCTGCACCCCACCCACTACAGC
ATCAGGAGCACCCTGAGGATGGAGCTGATGGGCTGTGACCTGAACAGCTGCAGCATGCCCCTGGGCATG
GAGAGCAAGGCCATCTCTGATGCCCAGATCACTGCCAGCAGCTACTTCACCAACATGTTTGCCACCTGG
AGCCCCAGCAAGGCCAGGCTGCACCTGCAGGGCAGGAGCAATGCCTGGAGGCCCCAGGTCAACAACCCC
AAGGAGTGGCTGCAGGTGGACTTCCAGAAGACCATGAAGGTGACTGGGGTGACCACCCAGGGGGTGAAG
AGCCTGCTGACCAGCATGTATGTGAAGGAGTTCCTGATCAGCAGCAGCCAGGATGGCCACCAGTGGACC
CTGTTCTTCCAGAATGGCAAGGTGAAGGTGTTCCAGGGCAACCAGGACAGCTTCACCCCTGTGGTGAAC
AGCCTGGACCCCCCCCTGCTGACCAGATACCTGAGGATTCACCCCCAGAGCTGGGTGCACCAGATTGCC
CTGAGGATGGAGGTGCTGGGCTGTGAGGCCCAGGACCTGTACTGA

In some embodiments, the ceDNA vector comprises an FVIII sequence that is at least 85% identical to the nucleic acid sequence as set forth in SEQ ID NO: 143. In some embodiments, the ceDNA vector comprises an FVIII sequence that is at least 90% identical to the nucleic acid sequence as set forth in SEQ ID NO: 143. In some embodiments, the ceDNA vector comprises an FVIII sequence that is at least 95% identical to the nucleic acid sequence as set forth in SEQ ID NO: 143. In some embodiments, the ceDNA vector comprises an FVIII sequence that is at least 96% identical to the nucleic acid sequence as set forth in SEQ ID NO: 143. In some embodiments, the ceDNA vector comprises an FVIII sequence that is at least 97% identical to the nucleic acid sequence as set forth in SEQ ID NO: 143. In some embodiments, the ceDNA vector comprises an FVIII sequence that is at least 98% identical to the nucleic acid sequence as set forth in SEQ ID NO: 143. In some embodiments, the ceDNA vector comprises an FVIII sequence that is at least 99% identical to the nucleic acid sequence as set forth in SEQ ID NO: 143. In some embodiments, the ceDNA vector comprises an FVIII sequence that consists of SEQ ID NO: 143.

### FVIII Therapeutic Proteins and Uses Thereof for the Treatment of Hemophilia A

The viral and non-viral vectors comprising the expression cassettes described herein can be used to deliver a liver-specific therapeutic protein (e.g., a FVIII protein) for treatment of hemophilia A associated with inappropriate expression of the liver-specific therapeutic protein (e.g., a FVIII protein) and/or mutations within the liver-specific therapeutic protein (e.g., a FVIII protein).

The vectors as described herein can be used to express any desired FVIII therapeutic protein. Exemplary therapeutic FVIII therapeutic proteins include but are not limited to any therapeutic protein (e.g., a FVIII protein), or portion thereof, expressed by, e.g., a nucleic acid at least 85%, at least 90%, at least 95%, at least 96%, at least 97%, at least 98% or at least 99% identical to SEQ ID NO: 143.

In one embodiment, the expressed FVIII therapeutic protein is functional for the treatment of a hemophilia A. In some embodiments, FVIII therapeutic protein does not cause an immune system reaction.

In another embodiment, the vectors encoding FVIII therapeutic protein or fragment thereof (e.g., functional fragment) can be used to generate a chimeric protein. Thus, it is specifically contemplated herein that a vector expressing a chimeric protein can be administered to e.g., to any one or more tissues selected from: liver, kidneys, gallbladder, prostate, adrenal gland. In some embodiments, when a vector that has been engineered to express FVIII is administered to an infant, or administered to a subject in utero, one can administer the vector to any one or more tissues selected from: liver, adrenal gland, heart, intestine, lung, and stomach, or to a liver stem cell precursor thereof for the in vivo or ex vivo treatment of hemophilia A.

### Hemophilia

Hemophilia A is a genetic deficiency in clotting factor VIII, which causes increased bleeding and usually affects males. In the majority of cases, it is inherited as an X-linked recessive trait, though there are cases which arise from spontaneous mutations. In terms of the symptoms of hemophilia A, there are internal or external bleeding episodes. Individuals with more severe hemophilia suffer more severe and more frequent bleeding, while others with mild hemophilia typically suffer more minor symptoms except after surgery or serious trauma. Moderate hemophiliacs have variable symptoms which manifest along a spectrum between severe and mild forms.

Current treatments to prevent bleeding in people with hemophilia A involve Factor VIII medication. Most individuals with severe hemophilia require regular supplementation with intravenous recombinant or plasma concentrate Factor VIII. Recombinant blood clotting factor VIII is one of the most complex proteins for industrial manufacturing due to the low efficiency of its gene transcription, massive intracellular loss of its proprotein during post-translational processing, and the instability of the secreted protein. Mild hemophiliacs can manage their condition with desmopressin, a drug which releases stored factor VIII from blood vessel walls.

There are many complications related to treatment of hemophilia A. In children, an easily accessible intravenous port can be inserted to minimize frequent traumatic intravenous cannulation. However, these ports are associated with high infection rate and a risk of clots forming at the tip of the catheter, rendering it useless. Viral infections can be common in hemophiliacs due to frequent blood transfusions which put patients at risk of acquiring blood borne infections, such as HIV, hepatitis B and hepatitis C. Prion infections can also be transmitted by blood transfusions. Another therapeutic complication of hemophilia A is the development of inhibitor antibodies against factor VIII due to frequent infusions. These develop as the body recognizes the infused factor VIII as foreign, as the body does not produce its own copy. In these individuals, activated factor VII, a precursor to factor VIII in the coagulation cascade, can be infused as a treatment for hemorrhage in individuals with hemophilia and antibodies against replacement factor VIII.

### Coagulation Cascade

Coagulation, also known as clotting, is the process by which blood changes from a liquid to a gel, forming a blood clot. It potentially results in hemostasis, the cessation of blood loss from a damaged vessel, followed by repair. The mechanism of coagulation involves activation, adhesion and aggregation of platelets along with deposition and maturation of fibrin. Disorders of coagulation are disease states which can result in bleeding (hemorrhage or bruising) or obstructive clotting (thrombosis).

Coagulation begins almost instantly after an injury to the blood vessel has damaged the endothelium lining the blood vessel. Exposure of blood to the subendothelial space initiates two processes: changes in platelets, and the exposure of subendothelial tissue factor to plasma Factor VII, which ultimately leads to fibrin formation. Platelets immediately form a plug at the site of injury; this is called primary hemostasis. Secondary hemostasis occurs simultaneously: additional coagulation factors or clotting factors beyond Factor VII (including Factor VIII) respond in a complex cascade to form fibrin strands, which strengthen the platelet plug.

The coagulation cascade of secondary hemostasis has two initial pathways which lead to fibrin formation. These are the contact activation pathway (also known as the intrinsic pathway), and the tissue factor pathway (also known as the extrinsic pathway), which both lead to the same fundamental reactions that produce fibrin. The primary pathway for the initiation of blood coagulation is the tissue factor (extrinsic) pathway. The pathways are a series of reactions, in which a zymogen (inactive enzyme precursor) of a serine protease and its glycoprotein co-factor are activated to become active components that then catalyze the next reaction in the cascade, ultimately resulting in cross-linked fibrin. Coagulation factors are generally indicated by Roman numerals, with a lowercase a appended to indicate an active form.

The coagulation factors are generally serine proteases (enzymes), which act by cleaving downstream proteins. The exceptions are tissue factor, FV, FVIII, FXIII. Tissue factor, FV and FVIII are glycoproteins, and Factor XIII is a transglutaminase. The coagulation factors circulate as inactive zymogens. The coagulation cascade is therefore classically divided into three pathways. The tissue factor and contact activation pathways both activate the “final common pathway” of factor X, thrombin and fibrin.

The main role of the tissue factor (extrinsic) pathway is to generate a “thrombin burst”, a process by which thrombin, the most important constituent of the coagulation cascade in terms of its feedback activation roles, is released very rapidly. FVIIa circulates in a higher amount than any other activated coagulation factor. The process includes the following steps:

Step 1: Following damage to the blood vessel, FVII leaves the circulation and comes into contact with tissue factor (TF) expressed on tissue-factor-bearing cells (stromal fibroblasts and leukocytes), forming an activated complex (TF-FVIIa).

Step 2: TF-FVIIa activates FIX and FX.

Step 3: FVII is itself activated by thrombin, FXIa, FXII and FXa.

Step 4: The activation of FX (to form FXa) by TF-FVIIa is almost immediately inhibited by tissue factor pathway inhibitor (TFPI).

Step 5: FXa and its co-factor FVa form the prothrombinase complex, which activates prothrombin to thrombin.

Step 6: Thrombin then activates other components of the coagulation cascade, including FV and FVIII (which forms a complex with FIX), and activates and releases FVIII from being bound to von Willebrand factor (vWF).

Step 7: FVIIIa is the co-factor of FIXa, and together they form the “tenase” complex, which activates FX; and so the cycle continues.

The contact activation (intrinsic) pathway begins with formation of the primary complex on collagen by high-molecular-weight kininogen (HMWK), prekallikrein, and FXII (Hageman factor). Prekallikrein is converted to kallikrein and FXII becomes FXIIa. FXIIa converts FXI into FXIa. Factor XIa activates FIX, which with its co-factor FVIIIa form the tenase complex, which activates FX to FXa. The minor role that the contact activation pathway has in initiating clot formation can be illustrated by the fact that patients with severe deficiencies of FXII, HMWK, and prekallikrein do not have a bleeding disorder. Instead, contact activation system is more involved in inflammation, and innate immunity.

The final common pathway shared by the intrinsic and extrinsic coagulation pathways involves the conversion of prothrombin into thrombin and fibrinogen into fibrin. Thrombin has a large array of functions, not only the conversion of fibrinogen to fibrin, the building block of a hemostatic plug. In addition, it is the most important platelet activator and on top of that it activates Factors VIII and V and their inhibitor protein C (in the presence of thrombomodulin), and it activates Factor XIII, which forms covalent bonds that crosslink the fibrin polymers that form from activated monomers.

Following activation by the contact factor or tissue factor pathways, the coagulation cascade is maintained in a prothrombotic state by the continued activation of FVIII and FIX to form the tenase complex, until it is down-regulated by the anticoagulant pathways.

In some embodiments, a vector for expression of a therapeutic protein (e.g., a FVIII protein) comprising an expression cassette as disclosed herein can also encode co-factors or other polypeptides, sense or antisense oligonucleotides, or RNAs (coding or non-coding; e.g., siRNAs, shRNAs, micro-RNAs, and their antisense counterparts (e.g., antagoMiR)) that can be used in conjunction with the Therapeutic protein (e.g., a FVIII protein) expressed from the ceDNA. Additionally, expression cassettes comprising sequence encoding an Therapeutic protein (e.g., a FVIII protein) can also include an exogenous sequence that encodes a reporter protein to be used for experimental or diagnostic purposes, such as β-lactamase, 3-galactosidase (LacZ), alkaline phosphatase, thymidine kinase, green fluorescent protein (GFP), chloramphenicol acetyltransferase (CAT), luciferase, and others well known in the art.

In one embodiment, the ceDNA vector comprises a nucleic acid sequence to express the therapeutic protein (e.g., a FVIII protein) that is functional for the treatment of hemophilia A. In a preferred embodiment, the therapeutic protein (e.g., a FVIII protein) does not cause an immune system reaction, unless so desired.

### VI. Pharmaceutical Compositions

In another aspect, pharmaceutical compositions are provided. The pharmaceutical composition comprises a ceDNA vector for expression of a therapeutic protein (e.g., a FVIII protein) as described herein and a pharmaceutically acceptable carrier or diluent.

The viral and nob-viral vectors for expression of a therapeutic protein (e.g., a FVIII protein) as disclosed herein can be incorporated into pharmaceutical compositions suitable for administration to a subject for in vivo delivery to cells, tissues, or organs of the subject. Typically, the pharmaceutical composition comprises a viral or non-viral vector (e.g., an AAV vector, a ceDNA vector) as disclosed herein and a pharmaceutically acceptable carrier. For example, the vectors for expression of a therapeutic protein (e.g., a FVIII protein) as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated.

In one embodiment, pharmaceutical compositions for therapeutic purposes can be formulated as a solution, microemulsion, dispersion, liposomes, or other ordered structure suitable to high vector concentration, in particular, high ceDNA vector concentration. Sterile injectable solutions can be prepared by incorporating the vector compound in the required amount in an appropriate buffer with one or a combination of ingredients enumerated above, as required, followed by filtered sterilization including a vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a vector (e.g., an AAV vector, a ceDNA vector) for expression of a therapeutic protein (e.g., a FVIII protein) can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high vector, in particular, high ceDNA vectorconcentration. Sterile injectable solutions can be prepared by incorporating the ceDNA vector compound in the required amount in an appropriate buffer with one or a combination of ingredients enumerated above, as required, followed by filtered sterilization.

Ator for expression of therapeutic protein (e.g., a FVIII protein) as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more vectors for expression of therapeutic protein (e.g., a FVIII protein) as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355, the contents of each of which are incorporated by reference in their entireties herein) and lipofection reagents are sold commercially (e.g., TRANSFECTAM™ and LIPFECTIN™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for expression of therapeutic protein (e.g., a FVIII protein) can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for expression of therapeutic protein (e.g., a FVIII protein) to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326, the contents of each of which are incorporated by reference in their entireties herein.

Nucleic acids, such as ceDNA vectors for expression of therapeutic protein (e.g., a FVIII protein) can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

Vectors (e.g., AAV vectors or ceDNA vectors) for expression of therapeutic protein (e.g., a FVIII protein) as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector for expression of therapeutic protein (e.g., a FVIII protein) as disclosed herein can be delivered into hematopoietic stem cells, for example, by the methods as described, for example, in U.S. Pat. No. 5,928,638, the contents of which is incorporated by reference in its entirety herein.

### VII. Methods of Use

A non-viral or viral vector for expression of a therapeutic protein (e.g., a FVIII protein) as disclosed herein can also be used in a method for the delivery of a nucleic acid sequence of interest (e.g., encoding a therapeutic protein (e.g., a FVIII protein)) to a target cell (e.g., a host cell). In some embodiments, the method comprises a method for delivering a therapeutic protein (e.g., a FVIII protein) to a cell of a subject in need thereof and treating hemophilia A. The disclosure allows for the in vivo expression of the therapeutic protein (e.g., a FVIII protein) encoded in the ceDNA vector in a cell in a subject such that therapeutic effect of the expression of the therapeutic protein (e.g., a FVIII protein) occurs. These results are seen with both in vivo and in vitro modes of vector delivery.

In some embodiments, the disclosure provides a method for the delivery of a therapeutic protein (e.g., a FVIII protein) in a cell of a subject in need thereof, comprising multiple administrations of the vector of the disclosure encoding said therapeutic protein (e.g., a FVIII protein). In some embodiments, the ceDNA vectors of the disclosure do not induce an immune response like that typically observed against encapsidated viral vectors, such that a multiple administration strategy will likely have greater success in a ceDNA-based system. The ceDNA vector are administered in sufficient amounts to transfect the cells of a desired tissue and to provide sufficient levels of gene transfer and expression of the therapeutic protein (e.g., a FVIII protein) without undue adverse effects.

The disclosure also provides for a method of treating hemophilia A in a subject comprising introducing into a target cell in need thereof (in particular a muscle cell or tissue) of the subject a therapeutically effective amount of a vector as described herein, optionally with a pharmaceutically acceptable carrier. While the vector can be introduced in the presence of a carrier, such a carrier is not required. The ceDNA vector selected comprises a nucleic acid sequence encoding an therapeutic protein (e.g., a FVIII protein) useful for treating hemophilia A.

The compositions and vectors provided herein can be used to deliver a therapeutic protein (e.g., a FVIII protein) for various purposes. In some embodiments, the transgene encodes an Therapeutic protein (e.g., a FVIII protein) that is intended to be used for research purposes, e.g., to create a somatic transgenic animal model harboring the transgene, e.g., to study the function of the therapeutic protein (e.g., a FVIII protein) product. In another example, the transgene encodes a therapeutic protein (e.g., a FVIII protein) that is intended to be used to create an animal model of hemophilia A. In some embodiments, the encoded therapeutic protein (e.g., a FVIII protein) is useful for the treatment or prevention of hemophilia A states in a mammalian subject. The therapeutic protein (e.g., a FVIII protein) can be transferred (e.g., expressed in) to a patient in a sufficient amount to treat hemophilia A associated with reduced expression, lack of expression or dysfunction of the gene.

In principle, the expression cassette can include a nucleic acid or any transgene that encodes a therapeutic protein (e.g., a FVIII protein) that is either reduced or absent due to a mutation or which conveys a therapeutic benefit when overexpressed is considered to be within the scope of the disclosure. Preferably, noninserted bacterial DNA is not present and preferably no bacterial DNA is present in the ceDNA compositions provided herein.

In another aspect, multiple vectors expressing different proteins or the same therapeutic protein (e.g., a FVIII protein) but operatively linked to different promoters or cis-regulatory elements can be delivered simultaneously or sequentially to the target cell, tissue, organ, or subject. Therefore, this strategy can allow for the gene therapy or gene delivery of multiple proteins simultaneously. It is also possible to separate different portions of a therapeutic protein (e.g., a FVIII protein) into separate vectors (e.g., different domains and/or co-factors required for functionality of a therapeutic protein (e.g., a FVIII protein)) which can be administered simultaneously or at different times, and can be separately regulatable, thereby adding an additional level of control of expression of a therapeutic protein (e.g., a FVIII protein).

The disclosure also provides for a method of treating hemophilia A in a subject comprising introducing into a target cell in need thereof (in particular a muscle cell or tissue) of the subject a therapeutically effective amount of a ceDNA vector as disclosed herein, optionally with a pharmaceutically acceptable carrier. While the ceDNA vector can be introduced in the presence of a carrier, such a carrier is not required. The ceDNA vector implemented comprises a nucleic acid sequence of interest useful for treating the hemophilia A. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### VIII. Methods of Delivery

In some embodiments, non-viral and viral vector for expression of a therapeutic protein as described herein can be delivered to a target cell in vitro or in vivo by various suitable methods. Vectors alone can be applied or injected. According to embodiments, the vectors can be delivered to a cell without the help of a transfection reagent or other physical means. Alternatively, according to other embodiments, the vectors for expression of a therapeutic protein (e.g., a FVIII protein) can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

One aspect of the technology described herein relates to a method of delivering a therapeutic protein (e.g., a FVIII protein) to a cell. Typically, for in vivo and in vitro methods, a non-viral or viral vector for expression of a therapeutic protein (e.g., a FVIII protein) as disclosed herein may be introduced into the cell using the methods as disclosed herein, as well as other methods known in the art. A vector for expression of a therapeutic protein (e.g., a FVIII protein) as disclosed herein are preferably administered to the cell in a biologically-effective amount. If the vector is administered to a cell in vivo (e.g., to a subject), a biologically-effective amount of the vector is an amount that is sufficient to result in transduction and expression of the therapeutic protein (e.g., a FVIII protein) in a target cell.

Exemplary modes of administration of a vector composition for expression of therapeutic protein (e.g., a FVIII protein) as disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular). Administration can be systemically or direct delivery to the liver or elsewhere (e.g., any kidneys, gallbladder, prostate, adrenal gland, heart, intestine, lung, and stomach).

Administration can be topical (e.g., to both skin and mucosal surfaces, including airway surfaces, and transdermal administration), intralymphatic, and the like, as well as direct tissue or organ injection (e.g., but not limited to, liver, but also to eye, muscles, including skeletal muscle, cardiac muscle, diaphragm muscle, or brain).

Methods for introduction of a nucleic acid vector for expression of therapeutic protein (e.g., a FVIII protein) as disclosed herein can be delivered into hematopoietic stem cells, for example, by the methods as described, for example, in U.S. Pat. No. 5,928,638, the contents of which is incorporated by reference in its entirety herein.

Administration of the vectors described herein (e.g., AAV, ceDNA) can be to any site in a subject, including, without limitation, a site selected from the group consisting of the liver and/or also eyes, brain, a skeletal muscle, a smooth muscle, the heart, the diaphragm, the airway epithelium, the kidney, the spleen, the pancreas, the skin.

The most suitable route in any given case will depend on the nature and severity of the condition being treated, ameliorated, and/or prevented and on the nature of the particular vector that is being used.

In one embodiment, delivery is to the liver. The vectors comprising the nucleic acids disclosed herein can be delivered to the liver through the hepatic artery, portal vein, or intravenously to produce therapeutic levels of therapeutic proteins or clotting factors in the blood. The capsid or vector is preferably suspended in a physiologically compatible transporter, and can be administered to a human or non-human mammalian patient. A person skilled in the art can easily select suitable transporters in view of the indication for which the transfer virus is directed. For example, a suitable carrier includes saline, which can be formulated with a variety of buffer solutions (eg, phosphate buffered saline). Other illustrative carriers include sterile saline, lactose, sucrose, calcium phosphate, gelatin, dextran, agar, pectin, sesame oil, and water.

In some embodiments, cells are removed from a subject, a ceDNA vector for expression of a therapeutic protein (e.g., a FVIII protein) as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of a therapeutic protein (e.g., a FVIII protein) as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, a ceDNA vector for expression of therapeutic protein (e.g., a FVIII protein) as disclosed herein can encode a therapeutic protein (e.g., a FVIII protein) as described herein (sometimes called a transgene or heterologous nucleic acid sequence) that is to be produced in a cell in vitro, ex vivo, or in vivo. For example, in contrast to the use of the ceDNA vectors described herein in a method of treatment as discussed herein, in some embodiments a ceDNA vector for expression of Therapeutic protein (e.g., a FVIII protein) may be introduced into cultured cells and the expressed Therapeutic protein (e.g., a FVIII protein) isolated from the cells, e.g., for the production of antibodies and fusion proteins. In some embodiments, the cultured cells comprising a ceDNA vector for expression of Therapeutic protein (e.g., a FVIII protein) as disclosed herein can be used for commercial production of antibodies or fusion proteins, e.g., serving as a cell source for small or large scale biomanufacturing of antibodies or fusion proteins. In alternative embodiments, a ceDNA vector for expression of Therapeutic protein (e.g., a FVIII protein) as disclosed herein is introduced into cells in a host non-human subject, for in vivo production of antibodies or fusion proteins, including small scale production as well as for commercial large scale Therapeutic protein (e.g., a FVIII protein) production.

The ceDNA vectors for expression of Therapeutic protein (e.g., a FVIII protein) as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### Dose Ranges

Provided herein are methods of treatment comprising administering to the subject an effective amount of a composition comprising a vector encoding a therapeutic protein (e.g., a FVIII protein) as described herein. As will be appreciated by a skilled practitioner, the term “effective amount” refers to the amount of the composition administered that results in expression of the therapeutic protein (e.g., a FVIII protein) in a “therapeutically effective amount” for the treatment of hemophilia A.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

for expression of therapeutic protein (e.g., a FVIII protein) as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a vector for expression of a therapeutic protein (e.g., a FVIII protein) as disclosed herein required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily, or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

An FVIII therapeutic protein can be expressed in a subject for at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. Long-term expression can be achieved by repeated administration of the ceDNA vectors described herein at predetermined or desired intervals.

The duration of treatment depends upon the subject's clinical progress and responsiveness to therapy. Continuous, relatively low maintenance doses are contemplated after an initial higher therapeutic dose.

### Unit Dosage Forms

In some embodiments, the pharmaceutical compositions comprising a viral or non-viral vector comprising an expression cassette as described herein, for expression of a therapeutic protein (e.g., a FVIII protein) as disclosed herein can conveniently be presented in unit dosage form. A unit dosage form will typically be adapted to one or more specific routes of administration of the pharmaceutical composition. In some embodiments, the unit dosage form is adapted for droplets to be administered directly to the eye. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for subretinal injection, suprachoroidal injection or intravitreal injection.

In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### IX. Methods of Treatment

The technology described herein also demonstrates methods for making, as well as methods of using the disclosed viral and non-viral vectors for expression of a therapeutic protein in a variety of ways, including, for example, ex vivo, ex situ, in vitro and in vivo applications, methodologies, diagnostic procedures, gene editing and/or gene therapy regimens to treat a subject suffering from a genetic disorder.

According to some embodiments, the subject is a human. According to some embodiments, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, progressive familial intrahepatic cholestasis (PFIC) type I (ATP8B1 deficiency), type II (ABCB11), type III (ABCB4), or type IV (TJP2) and Cathepsin A deficiency. According to some embodiments, the genetic disorder is Leber congenital amaurosis (LCA). According to some embodiments, the LCA is LCA10. According to some embodiments, the genetic disorder is Niemann-Pick disease. According to some embodiments, the genetic disorder is Stargardt macular dystrophy. According to some embodiments, the genetic disorder is glucose-6-phosphatase (G6Pase) deficiency (glycogen storage disease type I) or Pompe disease (glycogen storage disease type II). According to some embodiments, the genetic disorder is hemophilia A (Factor VIII deficiency). According to some embodiments, the genetic disorder is hemophilia B (Factor IX deficiency). According to some embodiments, the genetic disorder is hunter syndrome (Mucopolysaccharidosis II). According to some embodiments, the genetic disorder is cystic fibrosis. According to some embodiments, the genetic disorder is dystrophic epidermolysis bullosa (DEB). According to some embodiments, the genetic disorder is phenylketonuria (PKU). According to some embodiments, the genetic disorder is progressive familial intrahepatic cholestasis (PFIC). According to some embodiments, the genetic disorder is Wilson disease. According to some embodiments, the genetic disorder is Gaucher disease Type I, II or III.

In one embodiment, the expressed therapeutic protein (e.g., a FVIII protein) expressed from a vector as disclosed herein is functional for the treatment of disease. In a preferred embodiment, the therapeutic protein (e.g., a FVIII protein) does not cause an immune system reaction, unless so desired.

Provided herein is a method of treating hemophilia A in a subject comprising introducing into a target cell in need thereof (for example, a muscle cell or tissue, or other affected cell type) of the subject a therapeutically effective amount of a ceDNA vector for expression of therapeutic protein (e.g., a FVIII protein) as disclosed herein, optionally with a pharmaceutically acceptable carrier. While the vector can be introduced in the presence of a carrier, such a carrier is not required. The vector implemented comprises a nucleic acid sequence encoding a therapeutic protein (e.g., a FVIII protein) as described herein useful for treating the disease. In particular, a ceDNA vector for expression of therapeutic protein (e.g., a FVIII protein) as disclosed herein may comprise a desired therapeutic protein (e.g., a FVIII protein) DNA sequence operably linked to control elements capable of directing transcription of the desired therapeutic protein (e.g., a FVIII protein) encoded by the exogenous DNA sequence when introduced into the subject. The ceDNA vector for expression of therapeutic protein (e.g., a FVIII protein) as disclosed herein can be administered via any suitable route as provided above, and elsewhere herein.

Disclosed herein are ceDNA vector compositions and formulations for expression of Therapeutic protein (e.g., a FVIII protein) as disclosed herein that include one or more of the ceDNA vectors of the present disclosure together with one or more pharmaceutically-acceptable buffers, diluents, or excipients. Such compositions may be included in one or more diagnostic or therapeutic kits, for diagnosing, preventing, treating or ameliorating one or more symptoms of hemophilia A. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a viral or non-viral vector for expression of therapeutic protein (e.g., a FVIII protein) as disclosed herein, the method comprising providing to a cell, tissue or organ of a subject in need thereof, an amount of the vector as disclosed herein; and for a time effective to enable expression of the therapeutic protein (e.g., a FVIII protein) from the vector thereby providing the subject with a diagnostically- or a therapeutically-effective amount of the therapeutic protein (e.g., a FVIII protein) expressed by the vector. In a further aspect, the subject is human.

Another aspect of the technology described herein provides a method for diagnosing, preventing, treating, or ameliorating at least one or more symptoms of hemophilia A, a disorder, a dysfunction, an injury, an abnormal condition, or trauma in a subject. In an overall and general sense, the method includes at least the step of administering to a subject in need thereof one or more of the disclosed ceDNA vector for a therapeutic protein (e.g., a FVIII protein) production, in an amount and for a time sufficient to diagnose, prevent, treat or ameliorate the one or more symptoms of the disease, disorder, dysfunction, injury, abnormal condition, or trauma in the subject. In such an embodiment, the subject can be evaluated for efficacy of the therapeutic protein (e.g., a FVIII protein), or alternatively, detection of the therapeutic protein (e.g., a FVIII protein) or tissue location (including cellular and subcellular location) of the therapeutic protein (e.g., a FVIII protein) in the subject. As such, the ceDNA vector for expression of therapeutic protein (e.g., a FVIII protein) as disclosed herein can be used as an in vivo diagnostic tool, e.g., for the detection of cancer or other indications. In a further aspect, the subject is human.

Another aspect is use of a viral or non-viral vector for expression of a therapeutic protein (e.g., a FVIII protein) as disclosed herein as a tool for treating or reducing one or more symptoms of hemophilia A or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For unbalanced disease states, a vector for expression of a therapeutic protein (e.g., a FVIII protein) as disclosed herein can be used to create hemophilia A state in a model system, which could then be used in efforts to counteract the disease state. Thus, the vector for expression of a therapeutic protein (e.g., a FVIII protein) as disclosed herein permit the treatment of genetic diseases. As used herein, hemophilia A state is treated by partially or wholly remedying the deficiency or imbalance that causes the disease or makes it more severe.

As used herein, the term “therapeutically effective amount” is an amount of an expressed FVIII therapeutic protein, or functional fragment thereof that is sufficient to produce a statistically significant, measurable change in expression of a disease biomarker or reduction in a given disease symptom (see “Efficacy Measurement” below). Such effective amounts can be gauged in clinical trials as well as animal studies for a given ceDNA composition.

The efficacy of a given treatment for hemophilia A, can be determined by the skilled clinician. However, a treatment is considered “effective treatment,” as the term is used herein, if any one or all of the signs or symptoms of the disease or disorder is/are altered in a beneficial manner, or other clinically accepted symptoms or markers of disease are improved, or ameliorated, e.g., by at least 10% following treatment with a viral or non-viral vector encoding FVIII, or a functional fragment thereof. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or disorder; or (2) relieving the disease, e.g., causing regression of symptoms; and (3) preventing or reducing the likelihood of the development of the disease, or preventing secondary diseases/disorders associated with the disease, such as liver or kidney failure. An effective amount for the treatment of a disease means that amount which, when administered to a mammal in need thereof, is sufficient to result in effective treatment as that term is defined herein, for that disease.

Efficacy of an agent can be determined by assessing physical indicators that are particular to hemophilia A. Standard methods of analysis of hemophilia A indicators are known in the art.

### Host Cells

In some embodiments, a non-viral or viral vector for expression of a therapeutic protein (e.g., a FVIII protein) as disclosed herein delivers the therapeutic protein (e.g., a FVIII protein) transgene into a subject host cell.

In some embodiments, the cells are hepatic (i.e., liver) cells.

In some embodiments, the cells are photoreceptor cells. In some embodiments, the cells are RPE cells. In some embodiments, the subject host cell is a human host cell, including, for example blood cells, stem cells, hematopoietic cells, CD34⁺ cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including a non-viral or viral vector as disclosed herein, for expression of a therapeutic protein (e.g., a FVIII protein) as disclosed herein. Thus, one can use multiple host cells depending on the purpose as is obvious to the skilled artisan. A construct or a vector for expression of a therapeutic protein (e.g., a FVIII protein) as disclosed herein including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source.

The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered a vector for expression of a therapeutic protein (e.g., a FVIII protein) as disclosed herein ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

### Additional Diseases for Gene Therapy

In general, a viral or non-viral vector as described herein for expression of a therapeutic protein (e.g., a FVIII protein) as disclosed herein can be used to deliver any therapeutic protein in accordance with the description above to treat, prevent, or ameliorate the symptoms associated with aberrant protein expression or gene expression in a subject.

In some embodiments, a viral or non-viral vector for expression of a therapeutic protein as disclosed herein can be used to deliver a therapeutic protein to skeletal, cardiac or diaphragm muscle, for production of a therapeutic protein for secretion and circulation in the blood or for systemic delivery to other tissues to treat, ameliorate, and/or prevent a disease or disorder characterized by abberant gene expression.

Testing for Successful Gene Expression Using a ceDNA Vector

Assays well known in the art can be used to test the efficiency of gene delivery of a therapeutic protein (e.g., a FVIII protein) by a vector can be performed in both in vitro and in vivo models. Levels of the expression of the therapeutic protein (e.g., a FVIII protein) can be assessed by one skilled in the art by measuring mRNA and protein levels of the therapeutic protein (e.g., a FVIII protein) (e.g., reverse transcription PCR, western blot analysis, and enzyme-linked immunosorbent assay (ELISA)). In one embodiment, expression cassette comprises a reporter protein that can be used to assess the expression of the therapeutic protein (e.g., a FVIII protein), for example by examining the expression of the reporter protein by fluorescence microscopy or a luminescence plate reader. For in vivo applications, protein function assays can be used to test the functionality of a given therapeutic protein (e.g., a FVIII protein) to determine if gene expression has successfully occurred. One skilled will be able to determine the best test for measuring functionality of a therapeutic protein (e.g., a FVIII protein) expressed by the ceDNA vector in vitro or in vivo.

It is contemplated herein that the effects of gene expression of a therapeutic protein (e.g., a FVIII protein) from the vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, a therapeutic protein (e.g., a FVIII protein) in the expression cassette, expression construct, or non-viral or viral vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc.) or another publicly available database.

Determining Efficacy by Assessing Therapeutic Protein Expression from the Vector

Essentially any method known in the art for determining protein expression can be used to analyze expression of a therapeutic protein (e.g., a FVIII protein) from a viral or non-viral vector. Non-limiting examples of such methods/assays include enzyme-linked immunoassay (ELISA), affinity ELISA, ELISPOT, serial dilution, flow cytometry, surface plasmon resonance analysis, kinetic exclusion assay, mass spectrometry, Western blot, immunoprecipitation, and PCR.

For assessing a therapeutic protein (e.g., a FVIII protein) expression in vivo, a biological sample can be obtained from a subject for analysis. Exemplary biological samples include a biofluid sample, a body fluid sample, blood (including whole blood), serum, plasma, urine, saliva, a biopsy and/or tissue sample etc. A biological sample or tissue sample can also refer to a sample of tissue or fluid isolated from an individual including, but not limited to, tumor biopsy, stool, spinal fluid, pleural fluid, nipple aspirates, lymph fluid, the external sections of the skin, respiratory, intestinal, and genitourinary tracts, tears, saliva, breast milk, cells (including, but not limited to, blood cells), tumors, organs, and also samples of in vitro cell culture constituent. The term also includes a mixture of the above-mentioned samples. The term “sample” also includes untreated or pretreated (or pre-processed) biological samples. In some embodiments, the sample used for the assays and methods described herein comprises a serum sample collected from a subject to be tested.

### X. Various Applications of Viral and Non-Viral Vectors

As disclosed herein, the viral and non-viral vectors for expression of a therapeutic protein as described herein can be used to express a therapeutic protein for a range of purposes. In one embodiment, the vector expressing a therapeutic protein (e.g., a FVIII protein) can be used to create a somatic transgenic animal model harboring the transgene, e.g., to study the function or disease progression of hemophilia A. In some embodiments, a ceDNA vector expressing a therapeutic protein (e.g., a FVIII protein) is useful for the treatment, prevention, or amelioration of hemophilia A states or disorders in a mammalian subject.

In some embodiments the therapeutic protein (e.g., a FVIII protein) can be expressed from the vector in a subject in a sufficient amount to treat a disease associated with increased expression, increased activity of the gene product, or inappropriate upregulation of a gene.

In some embodiments the therapeutic protein (e.g., a FVIII protein) can be expressed from the vector in a subject in a sufficient amount to treat hemophilia A with a reduced expression, lack of expression or dysfunction of a protein.

It will be appreciated by one of ordinary skill in the art that the transgene may not be an open reading frame of a gene to be transcribed itself; instead it may be a promoter region or repressor region of a target gene, and the ceDNA vector may modify such region with the outcome of so modulating the expression of the FVIII gene.

The compositions and viral and non-viral vectors for expression of a therapeutic protein (e.g., a FVIII protein) as disclosed herein can be used to deliver a therapeutic protein (e.g., a FVIII protein) for various purposes as described above.

In some embodiments, the transgene encodes one or more therapeutic proteins which are useful for the treatment, amelioration, or prevention of hemophilia A states in a mammalian subject. The therapeutic protein (e.g., a FVIII protein) expressed by the vector is administered to a patient in a sufficient amount to treat hemophilia A associated with an abnormal gene sequence, which can result in any one or more of the following: increased protein expression, over activity of the protein, reduced expression, lack of expression or dysfunction of the target gene or protein.

In some embodiments, the vectors for expression of a therapeutic protein (e.g., a FVIII protein) as disclosed herein are envisioned for use in diagnostic and screening methods, whereby a therapeutic protein (e.g., a FVIII protein) is transiently or stably expressed in a cell culture system, or alternatively, a transgenic animal model.

Another aspect of the technology described herein provides a method of transducing a population of mammalian cells with a ceDNA vector for expression of a therapeutic protein (e.g., a FVIII protein) as disclosed herein. In an overall and general sense, the method includes at least the step of introducing into one or more cells of the population, a composition that comprises an effective amount of one or more of the ceDNA vectors for expression of a therapeutic protein (e.g., a FVIII protein) as disclosed herein.

Additionally, the present disclosure provides compositions, as well as therapeutic and/or diagnostic kits that include one or more of the disclosed ceDNA vectors for expression of Therapeutic protein (e.g., a FVIII protein) as disclosed herein or ceDNA compositions, formulated with one or more additional ingredients, or prepared with one or more instructions for their use.

A cell to be administered a ceDNA vector for expression of a therapeutic protein as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

Production and Purification of ceDNA Vectors Expressing a Therapeutic Protein

The viral and non-viral vectors disclosed herein are to be used to produce a therapeutic protein (e.g., a FVIII protein) either in vitro or in vivo. The therapeutic protein (e.g., a FVIII protein) that is produced in this manner can be isolated, tested for a desired function, and purified for further use in research or as a therapeutic treatment.

Each system of protein production has its own advantages/disadvantages. While proteins produced in vitro can be easily purified and can proteins in a short time, proteins produced in vivo can have post-translational modifications, such as glycosylation.

A therapeutic protein produced using viral and non-viral vectors described herein can be purified using any method known to those of skill in the art, for example, ion exchange chromatography, affinity chromatography, precipitation, or electrophoresis.

A therapeutic protein produced by the methods and compositions described herein can be tested for binding to the desired target protein.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims.

### Examples

The following examples are provided by way of illustration not limitation.

### Example 1: In Silico Analyses for Identification of Potentially Improved Human Liver-Specific Promoter

The human SERPINA1 enhancer (hSerpEnh) is often used to drive liver-specific gene expression (Chuah et al. (2014) Mol Ther 22(9): 1605-1613). Multiple bioinformatic analyses were used to inform modification of the hSerpEnh for improved function and are described below.

### Analysis of Evolutionary Conservation

Cis-regulatory regions with similar sequence and similar sequence contexts often have conserved function but distinct performance attributes. A curated collection of more than 100 vertebrate genomes were analyzed to identify a set of predicted functionally conserved enhancers with divergent sequence. The function of a range of these enhancer elements were assessed to identify higher-expressing modules. Selections were also prioritized based on amount of CpG content and poly C and poly G sequence motifs.

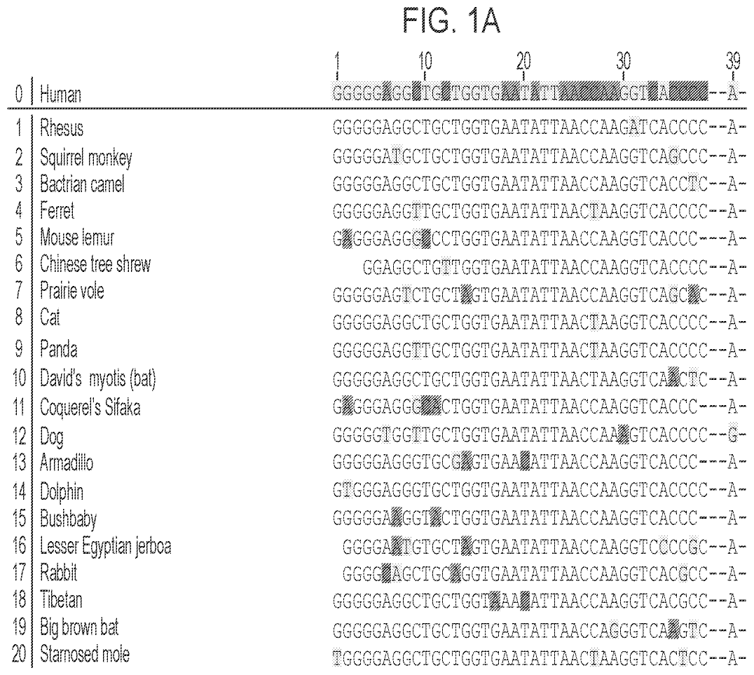

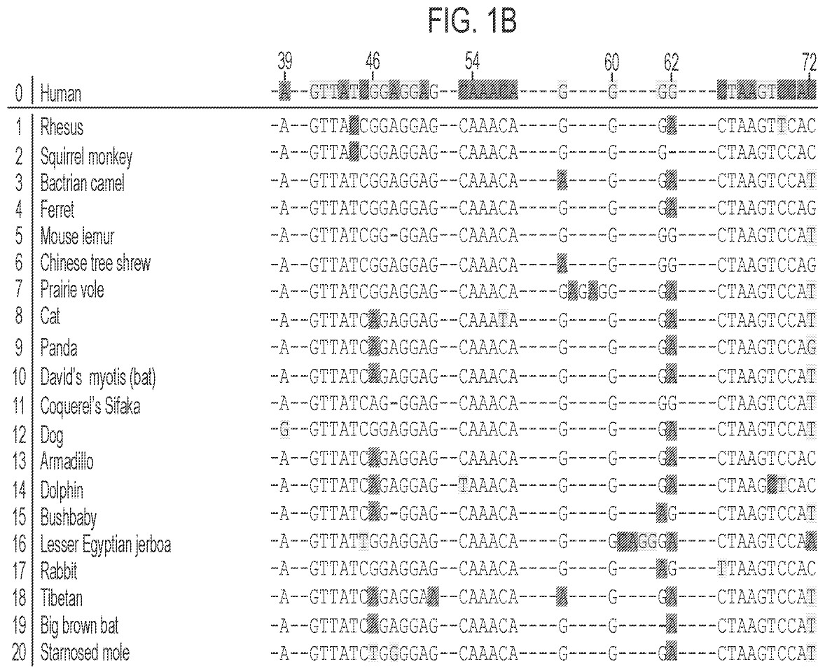

20 homologous sequences of human SERPINA1 enhancer region were identified and selected (see FIGS. 1A-1B) to screen for SerpEnh variants with improved expression characteristics. These sequences are listed in Table 4. FIG. 1A and FIG. 1B depict SERPINA1 sequences and alignment of conserved enhancer regions of human and 20 other vertebrates. 115 non-human vertebrate genomes were initially assessed for conserved SERPINA1 enhancer regions using the UCSC multiz100way and multiz30way multiple alignments. Depicted in FIGS. 1A-1B are the conserved SERPINA1 enhancer regions from 20 vertebrates with >90% identity to the human SERPINA1 enhancer, which are mapped to the human SERPINA1 enhancer sequence with Geneious. Highlighted nucleotides in the aligned sequences represent differences from the human reference sequence. Identification and modification of non-Consensus Transcription Factor Binding Sites (TFBS)

Transcription factor binding sites can be identified by in silico analysis and represent one sequence across a family of possible functional sites that is often divergent from the known consensus sequence. Several important liver-specific transcription factor binding sites were identified that diverged from consensus.

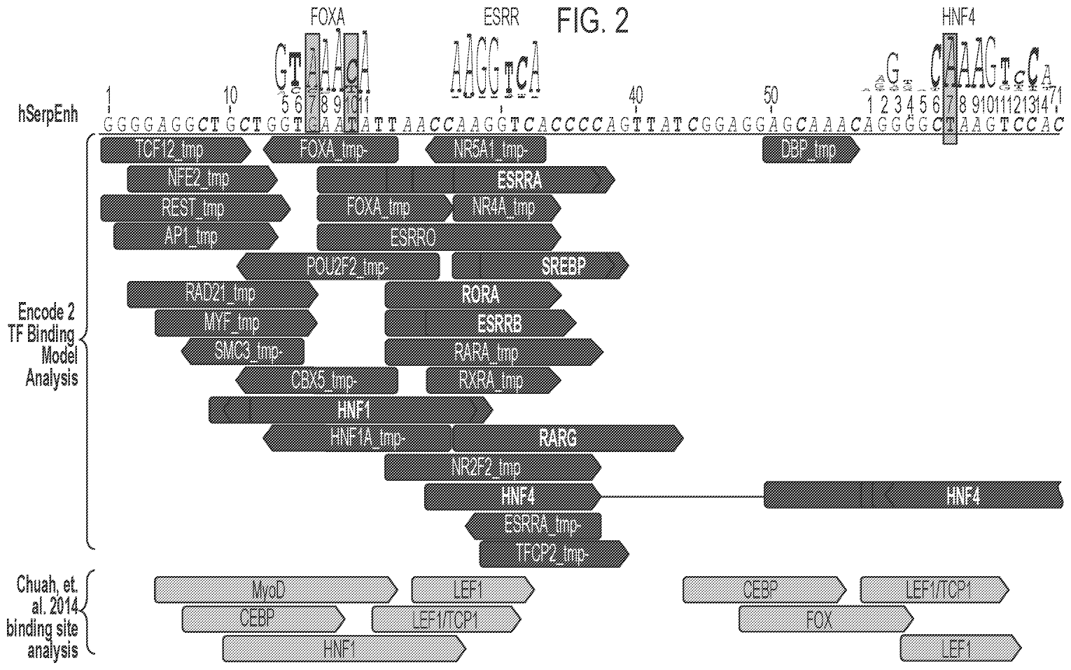

The hSerpEnh contains near-consensus binding sites for many transcription factors, including HNF4 and FOXA, which are key regulators of hepatic gene expression (see FIG. 2). Orange arrows represent TF binding motifs described by Chuah et al. (2014). Red arrows represent TF binding motifs identified by our independent analysis (FIG. 2). FIMO (Bailey et al. (2009) Nucleic Acids Res 37: W202-W208) was used to scan the human SERPINA1 enhancer sequence with position weight matrices for TFs generated by the ENCODE Project. Representative motif matches with p<1e-4 are displayed. Sequence logos representing position weight matrices for FOXA (JASPAR MA0148.3), ERR2 (HOCOMOCO ERR2_HUMAN.H11MO.0.A), and HNFA (HNF4A_HUMAN.H11MO.0.A) are shown above the corresponding motifs identified in our analysis (FIG. 2). Positions where the human SERPINA1 sequence differs from the most highly preferred nucleotide in the sequence logos are boxed and highlighted in red (FIG. 2).

Off-consensus nucleotides were modified to reinstate the consensus sequences based on the hypothesis that they would result in higher affinity for the transcription factor and drive higher levels of transcription initiation.

### CpG Content Minimization

Promoters often contain CpG dinucleotides that are undesirable for gene therapy applications. CpGs can impact expression durability through stimulation of the innate immune system and through methylation-based silencing. Nevertheless, removal of CpGs from cis-regulatory regions is non-trivial as they often play important functional roles in driving expression.

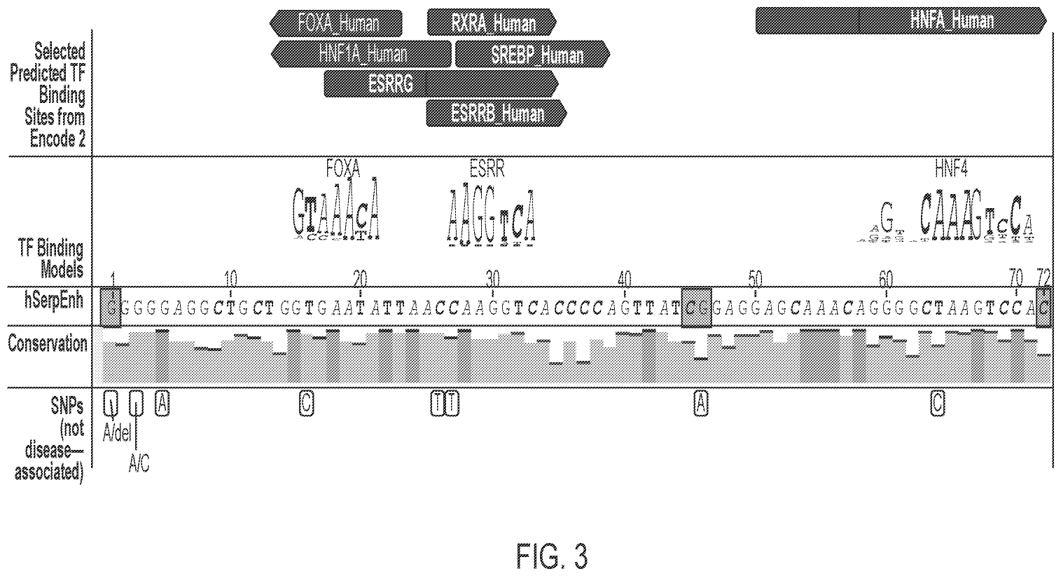

Multiple bioinformatic analyses were employed to inform removal of CpG from hSerpEnh (i.e., CpG ablation) (see FIG. 3). The evolutionary conservation analysis provided a rational path for selective removal of CpGs in the enhancer without disrupting function. Enhancer regions from diverse species that did not contain some or all CpGs but were likely to maintain function were identified. The human SERPINA1 enhancer contains one internal CpG and the potential to form CpGs at its 5′ and 3′ ends (highlighted in red and boxed in the “hSerpEnh” track). Low sequence conservation, the presence of human SNPs that are not known to be associated with disease, and the absence of predicted transcription factor (TF) binding sites were assessed to inform sequence changes to ablate the central CpG and the remove potential for CpG formation at the ends of the sequence.

Further, it was hypothesized that positions within the human SERPINA1 enhancer that are poorly conserved between species are less consequential to the enhancer's function, and thus better targets for sequence modifications that lead to CpG ablations. To assess sequence conservation at each position in the human SERPINA1 enhancer, 115 non-human vertebrate genomes were evaluated for conserved SERPINA1 enhancer elements in the UCSC multiz100way and multiz30way multiple alignments. Of these 115 genomes, 43 contained conserved SERPINA1 enhancer regions, which were aligned using the MUSCLE alignment algorithm. The “Conservation” track displays the mean pairwise identity between all pairs of nucleotides in the aligned sequences at each position in the enhancer. Green bars represent 100% identity and dark yellow bars represent 30 to <100% identity. Nucleotides that differed from the human sequence, but were utilized in the aligned position in other genomes were preferentially used for CpG ablations. It was further hypothesized that positions in CpGs that contain non-disease associated SNPs in the human population would be preferable targets for CpG ablation, and that changing the sequence to match non-disease associated SNPs would minimize changes to enhancer function. The “SNPs” track depicts SNPs within the human SERPINA1 enhancer that are cataloged in the 1000 Genomes Project and dbSNP that are not known to be associated with disease. Changes were made to ensure that the SERPINA1 sequence to ablate CpGs did not interfere with predicted transcription binding (TF) sites. The top track depicts selected TF motifs from our motif analysis (described in FIG. 2), in which it was found that TF motif clusters and the motifs for TFs that play key roles in hepatic expression had minimal overlap with the internal CpG.

CpG-free elements were either tested directly for function or used as a reference for making functionally permissive substitutions in the native human enhancer region.

The variants of SerpEnh generated from the bioinformatic analyses above are listed in the tables below, e.g., Table 4.

The results are described in the following Examples.

TABLE 3
Selected conserved SERPINA1 enhancer variants from human and 20 other vertebrates

| | | | SEQ ID |
|---|---|---|---|
| Name | Species | SERPINA1 enhancer region sequence | NO: |
| hSerpEnh | Human | GGGGGAGGCTGCTGGTGAATATTAACCAAGGTCACCCCAGTTATC | 137 |
| | | GGAGGAGCAAACAGGGGCTAAGTCCAC | |
| SerpEnh_Rhesus | Rhesus | GGGGGAGGCTGCTGGTGAATATTAACCAAGATCACCCCAGTTACC | 117 |
| | | GGAGGAGCAAACAGGGACTAAGTTCAC | |
| SerpEnh_Squirrel_ | Squirrel monkey | GGGGGATGCTGCTGGTGAATATTAACCAAGGTCAGCCCAGTTACC | 118 |
| monkey | | GGAGGAGCAAACAGGGCTAAGTCCAC | |
| SerpEnh_Bactrian_ | Bactrian camel | GGGGGAGGCTGCTGGTGAATATTAACCAAGGTCACCTCAGTTATC | 119 |
| camel | | GGAGGAGCAAACAAGGACTAAGTCCAT | |
| SerpEnh_Ferret | Ferret | GGGGGAGGTTGCTGGTGAATATTAACTAAGGTCACCCCAGTTATC | 120 |
| | | GGAGGAGCAAACAGGGACTAAGTCCAG | |
| SerpEnh_Mouse_ | Mouse lemur | GAGGGAGGGCGCTGGTGAATATTAACCAAGGTCACCCAGTTATCG | 121 |
| lemur | | GGGAGCAAACAGGGGCTAAGTCCAT | |
| SerpEnh_Chinese_ | Chinese tree shrew | GGAGGCTGTTGGTGAATATTAACCAAGGTCACCCCAGTTATCGGA | 82 |
| tree_shrew | | GGAGCAAACAAGGGCTAAGTCCAC | |
| SerpEnh_Prairie_ | Prairie vole | GGGGGAGTCTGCTAGTGAATATTAACCAAGGTCAGCACAGTTATC | 123 |
| vole | | GGAGGAGCAAACAGAGAGGGACTAAGTCCAT | |
| SerpEnh_Cat | Cat | GGGGGAGGCTGCTGGTGAATATTAACTAAGGTCACCCCAGTTATC | 124 |
| | | AGAGGAGCAAATAGGGACTAAGTCCAT | |
| SerpEnh_Panda | Panda | GGGGGAGGTTGCTGGTGAATATTAACTAAGGTCACCCCAGTTATC | 125 |
| | | AGAGGAGCAAACAGGGACTAAGTCCAG | |
| SerpEnh_David's_ | David's myotis | GGGGGAGGCTGCTGGTGAATATTAACCAAGGTCAACTCAGTTATC | 126 |
| myotis | | AGAGGAGCAAACAGGGACTAAGTCCAT | |
| SerpEnh_Coquerel's_ | Coquerel's sifaka | GAGGGAGGGCACTGGTGAATATTAACCAAGGTCACCCAGTTATCG | 127 |
| sifaka | | GGGAGCAAACAGGGGCTAAGTCCAT | |
| SerpEnh_Dog | Dog | GGGGGTGGTTGCTGGTGAATATTAACCAAAGTCACCCCGGTTATC | 128 |
| | | GGAGGAGCAAACAGGGACTAAGTCCAT | |
| SerpEnh_Armadillo | Armadillo | GGGGGAGGCTGCGAGTGAACATTAACCAAGGTCACCCAGTTATCA | 129 |
| | | GAGGAGCAAACAGGGACTAAGTCCAC | |
| SerpEnh_Dolphin | Dolphin | GTGGGAGGCTGCTGGTGAATATTAACCAAGGTCACCCCAGTTATC | 130 |
| | | AGAGGAGTAAACAGGGACTAAGCTCAC | |
| SerpEnh_Bushbaby | Bushbaby | GGGGGAAGCTACTGGTGAATATTAACCAAGGTCACCCAGTTATCA | 131 |
| | | GGGAGCAAACAGGAGCTAAGTCCAT | |
| SerpEnh_Lesser_ | Lesser Egyptian | GGGGAATCTGCTAGTGAATATTAACCAAGGTCCCCGCAGTTATTG | 132 |
| Egyptian_jerboa | jerboa | GAGGAGCAAACAGGCAGGGACTAAGTCCAA | |
| SerpEnh_Rabbit | Rabbit | GGGGCAGCTGCAGGTGAATATTAACCAAGGTCACGCCAGTTATCG | 133 |
| | | GAGGAGCAAACAGGAGTTAAGTCCAC | |
| SerpEnh_Tibetan_ | Tibetan antelope | GGGGGAGGCTGCTGGTAAACATTAACCAAGGTCACCCCAGTTATC | 134 |
| antelope | | AGAGGAACAAACAAGGACTAAGTCCAT | |
| SerpEnh_Big_ | Big brown bat | GGGGGAGGCTGCTGGTGAATATTAACCAGGGTCAACTCAGTTATC | 135 |
| brown_bat | | AGAGGAGCAAACAGGACTAAGTCCAT | |
| SerpEnh_ | Starnosed mole | TGGGGAGGCTGCTGGTGAATATTAACTAAGGTCACTCCAGITATC | 136 |
| Starnosed_mole | | TGGGGAGCAAACAGGGACTAAGTCCAT | |

TABLE 4
Variants of human SERPINA1 enhancer (hSerpEnh) based on bioinformatic analyses

| | | | SEQ ID |
|---|---|---|---|
| Name | Description | SERPINA1 enhancer region sequence | NO: |
| hSerpEnh_100_ | hSerpEnh with modifications based on the | GGGGGAGGCTGCTGGTGAATATTAACCAAGATCACCCCA | 111 |
| vertebrate_consensus_ | consensus sequence from the UCSC 100- | GTTATCAGAGGAGCAAACAGGGACTAAGTCCAT | |
| v1 | vertebrate and 27-primate multiple alignments | | |
| | version 1 | | |
| hSerpEnh_100_ | HSerpEnh with modifications based on the | GGGGGAGGCTGCTGGTGAATATTAACCAAGGTCACCCCA | 112 |
| vertebrate_consensus_ | consensus sequence from the UCSC 100- | GTTACCAGAGGAGCAAACAGGGACTAAGTCCAT | |
| v2 | vertebrate and 27-primate multiple alignments | | |
| | version 2 | | |
| hSerpEnh_100_ | HSerpEnh with modifications based on the | GGGGGAGGCTGCTGGTGAATATTAACCAAGGTCACCCCA | 113 |
| vertebrate_consensus_ | consensus sequence from the UCSC 100- | GTTATCAGAGGAGCAAACAGGGACTAAGTTCAT | |
| v3 | vertebrate and 27-primate multiple alignments | | |
| | version 3 | | |
| hSerpEnh_100_ | HSerpEnh with modifications based on the | AGGGGAGGCTGCTGGTGAATATTAACCAAGGTCACCCCA | 114 |
| vertebrate_consensus_ | consensus sequence from the UCSC 100- | GTTATCAGAGGAGCAAACAGGGACTAAGTCCAT | |
| v4 | vertebrate and 27-primate multiple alignments | | |
| | version 4 | | |
| hSerpEnh_FOXA_ | HSerpEnh with FOXA consensus site version 1 | AGGGGAGGCTGCTGGTAAACATTAACCAAGGTCACCCCA | 86 |
| consensus_v1 | | GTTATCAGAGGAGCAAACAGGGGCTAAGTCCAC | |
| hSerpEnh_FOXA_ | HSerpEnh with FOXA consensus site version 2 | AGGGGAGGCTGCTGGTAAATATTAACCAAGGTCACCCCA | 87 |
| consensus_v2 | | GTTATCAGAGGAGCAAACAGGGGCTAAGTCCAT | |
| hSerpEnh_FOXA_ | HSerpEnh with FOXA & HNF4 consensus sites | GGGGGAGGCTGCTGGTAAACATTAACCAAGGTCACCCCA | 88 |
| HNF4_consensus_v1 | version 1 | GTTATCAGAGGAGCAAACAGGGGCAAAGTCCAC | |
| hSerpEnh_FOXA_ | HSerpEnh with FOXA & HNF4 consensus sites | AGGGGAGGCTGCTGGTAAATATTAACCAAGGTCACCCCA | 89 |
| HNF4_consensus_v2 | version 2 | GTTATCAGAGGAGCAAACAGGGGCAAAGTCCAC | |
| hSerpEnh_HNF1_ | HSerpEnh with HNF1 consensus site version 1 | AGGGGAGGCTGCTGGTTAATGATTAACTAAGGTCACCCC | 90 |
| consensus_v1 | | AGTTATCAGAGGAGCAAACAGGGGCTAAGTCCAC | |
| hSerpEnh_HNF1_ | HSerpEnh with HNF1 consensus site version 2 | AGGGGAGGCTGCTGGTTAATCATTAACTAAGGTCACCCC | 91 |
| consensus_v2 | | AGTTATCAGAGGAGCAAACAGGGGCTAAGTCCAC | |
| hSerpEnh_HNF1_ | HSerpEnh with HNF1 & HNF4 consensus sites | GGGGGAGGCTGCTGGTTAATGATTAACTAAGGTCACCCC | 92 |
| HNF4_consensus_v1 | version 1 | AGTTATCAGAGGAGCAAACAGGGGCAAAGTCCAC | |
| hSerpEnh_HNF1_ | HSerpEnh with HNF1 & HNF4 consensus sites | GGGGGAGGCTGCTGGTTAATCATTAACTAAGGTCACCCC | 93 |
| HNF4_consensus_v2 | version 2 | AGTTATCAGAGGAGCAAACAGGGGCAAAGTCCAC | |
| hSerpEnh_HNF4_ | HSerpEnh with HNF4 consensus site version 1 | AGGGGAGGCTGCTGGTGAATATTAACCAAGGTCACCCCA | 94 |
| consensus_v1 | | GTTATCAGAGGAGCAAACAGGGGCAAAGTCCAT | |
| hSerpEnh_HNF4_ | HSerpEnh with HNF4 consensus site version 2 | AGGGGAGGCTGCTGGTGAATATTAACCAAGGTCACCTCA | 95 |
| consensus_v2 | | GTTATCAGAGGAGCAAACAGGGGCAAAGTCCAC | |
| hSerpEnh_human_ | HSerpEnh with modifications based on non- | AGAGGAGGCTGCTGGTGAATATTAACTAAGGTCACCCCA | 96 |
| SNPs_v1 | disease associated human SNPs version 1 | GTTATCAGAGGAGCAAACAGGGGCTAAGTCCAC | |
| hSerpEnh_human_ | HSerpEnh with modifications based on non- | AGAGAAGGCTGCTGGTGAATATTAACTAAGGTCACCCCA | 97 |
| SNPS_v2 | disease associated human SNPs version 2 | GTTATCGGAGGAGCAAACAGGGGCTAAGTCCAC | |
| hSerpEnh_low_TFBS_ | HSerpEnh with modifications to end regions | GGGGGAGGCTGCTGGTGAATATTAACCAAGGTCAGCACA | 98 |
| and_end_regions_v1 | and regions with fewer predicted transcription | GTTATCAGAGGAGCAAACAGGGGCTAAGTCCAT | |
| | factor binding sites version 1 | | |
| hSerpEnh_low_TFBS_ | HSerpEnh with modifications to end regions | GGGGGAGGCTGCTGGTGAATATTAACCAAGGTCAACTCA | 99 |
| and_end_regions_v2 | and regions with fewer predicted transcription | GTTATCAGAGGAGCAAACAGGGGCTAAGTCCAT | |
| | factor binding sites version 2 | | |
| hSerpEnh_low_TFBS_ | HSerpEnh with modifications to end regions | AGGGGAGGCTGCTGGTGAATATTAACCAAGGTCACCTCA | 100 |
| and_end_regions_v3 | and regions with fewer predicted transcription | GTTATCAGAGGAGCAAACAGGGGCTAAGTCCAT | |
| | factor binding sites version 3 | | |
| hSerpEnh_low_TFBS_ | HSerpEnh with modifications to end regions | AGGGGAGGCTGCTGGTGAATATTAACCAAGGTCAGCCCA | 101 |
| and_end_regions_v4 | and regions with fewer predicted transcription | GTTATTGGAGGAGCAAACAGGGGCTAAGTCCAT | |
| | factor binding sites version 4 | | |
| hSerpEnh_low_TFBS_ | HSerpEnh with modifications to end regions | AGGGGAGGCTGCTGGTGAATATTAACCAAGGTCACCCCA | 102 |
| and_end_regions_v5 | and regions with fewer predicted transcription | GTTATTAGAGGAGCAAACAGGGGCTAAGTCCAT | |
| | factor binding sites v5 | | |
| hSerpEnh_low_TFBS_ | HSerpEnh with modifications to end regions | AGGGGAGGCTGCTGGTGAATATTAACCAAGGTCACCCCA | 103 |
| and_end_regions_v6 | and regions with fewer predicted transcription | GTTACTGGAGGAGCAAACAGGGGCTAAGTCCAT | |
| | factor binding sites v6 | | |
| hSerpEnh_low_TFBS_ | HSerpEnh with modifications to regions with | GGGGGAGGCTGCTGGTGAATATTAACCAAGGTCAGCACA | 104 |
| region_v1 | fewer predicted transcription factor binding | GTTACCAGAGGAGCAAACAGGGGCTAAGTCCAC | |
| | sites version 1 | | |
| hSerpEnh_low_TFBS_ | HSerpEnh with modifications to regions with | GGGGGAGGCTGCTGGTGAATATTAACCAAGGTCAACTCA | 105 |
| region_v2 | fewer predicted transcription factor binding | GTTACCAGAGGAGCAAACAGGGGCTAAGTCCAC | |
| | sites version 2 | | |
| hSerpEnh_low_TFBS_ | HSerpEnh with modifications to regions with | GGGGGAGGCTGCTGGTGAATATTAACCAAGGTCACCCCA | 106 |
| region_v3 | fewer predicted transcription factor binding | GTTACTAGGGGAGCAAACAGGGGCTAAGTCCAC | |
| | sites version 3 | | |
| hSerpEnh_low_TFBS_ | HSerpEnh with modifications to regions with | GGGGGAGGCTGCTGGTGAATATTAACCAAGGTCACCCCA | 107 |
| region_v4 | fewer predicted transcription factor binding | GTTACTAGAGGAACAAACAGGGGCTAAGTCCAC | |
| | sites version 4 | | |
| hSerpEnh_low_TFBS_ | HSerpEnh with modifications to regions with | GGGGGAGGCTGCTGGTGAATATTAACCAAGGTCACCCCA | 108 |
| region_v5 | fewer predicted transcription factor binding | GTTATTAGGGGAACAAACAGGGGCTAAGTCCAC | |
| | sites v5 | | |
| hSerpEnh_11_NHP_ | HSerpEnh with modifications based on the | GGGGGAGGCTGCTGGTGAATATTAACCAAGGTCACCCCA | 109 |
| consensus_v1 | Rhesus SERPINA1 enhancer sequence, which | GTTACCAGAGGAGCAAACAGGGACTAAGTTCAC | |
| | is shared with at least 10 other non-human | | |
| | primate version 1 | | |
| hSerpEnh_11_NHP_ | HSerpEnh with modifications based on the | GGGGGAGGCTGCTGGTGAATATTAACCAAGGTCACCCCA | 110 |
| consensus_v2 | Rhesus SERPINA1 enhancer sequence, which | GTTACCGGAGGAGCAAACAGGGACTAAGTTCAT | |
| | is shared with at least 10 other non-human | | |
| | primate version 2 | | |
| hSerpEnh_end_regions_ | HSerpEnh with modifications to end regions | AAGGGAGGCTGCTGGTGAATATTAACCAAGGTCACCCCA | 115 |
| v1 | version 1 | GTTATCGGAGGAGCAAACAGGGGCTAAGTTCAT | |
| hSerpEnh_end_regions_ | HSerpEnh with modifications to end regions | AAGGGAGGCTGCTGGTGAATATTAACCAAGGTCACCCCA | 116 |
| v2 | version 2 | GTTATCGGAGGAGCAAACAGGGACTAAGTCCAT | |

### Example 2: In Vitro Activity of Single Variants of Human SERPINA1 Enhancer

### Dual Luciferase Transient Transfection Assay

Promega ViaFect Transfection: Efficacy of enhancer variants were evaluated in vitro using luciferase reporter assays. Expression plasmids containing enhancer variants were transfected into HepG2 cells using Promega ViaFect Transfection. Briefly, 24 hr before beginning transfections, 25,000-30,000 HepG2 cells/well were seeded in 96-well collagen-I coated plates in 100 μL DMEM+10% FBS and incubated at 37C with 5% humidity. DNA master mixes for each experimental plasmid to be transfected were prepared. Transfections were performed in triplicate (3 wells/plasmid), unless otherwise noted. For each well to be transfected, 1 ng NanoLuc pNL1.1.TK[Nluc/TK] plasmid, 67 ng experimental firefly luciferase plasmid and 133 ng pGEM®-3Zf(−) carrier plasmid were mixed and brought to a volume of 9.2 μL with Opti-MEM. Next, 0.8 μL room temperature ViaFect per well was added to each DNA master mix and incubated 5-20 min at room temperature. Each well was transfected with 10 μL of ViaFect/Opti-MEM/NanoLuc mastermix and incubated at 37° C. with 5% humidity for 24 hr prior to performing luminescence assay. Benchmarking plasmids, 1× hSerpEnh-Firefly luciferase or 3× hSerpEnh-Firefly luciferase, were included on every plate.

Promega NanoGlo Dual Luciferase Assay: 24 hr post-transfection, media was replaced with 80 μL room temperature PBS to prevent phenol red from interfering with the assay. Plates were allowed to equilibrate to room temperature. ONE-Glo™ EX Reagent was prepared as follows: the contents of one bottle of ONE-Glo™ EX Luciferase Assay Buffer was transferred to one bottle of ONE-Glo™ EX Luciferase Assay Substrate and mixed by inversion until the substrate was thoroughly dissolved. 80 μL of ONE-Glo™ EX Reagent was added to each well. Samples were mixed by shaking on an orbital shaker for 3 min at 500 rpm. 140 μL of lysed cells was transferred to a white 96-well plate to minimize cross-talk between wells and absorption of the emitted light. Firefly luciferase luminescence was measured on a SpectraMax M5 plate reader. The NanoDLR™ Stop & GloR Substrate was diluted 1:100 into an appropriate volume of room-temperature NanoDLR™ Stop & GloR Buffer and mixed by inversion. 70 μL of NanoDLR™ Stop & GloR Reagent was added to each well, shaken for 3 min at 700 rpms and incubated for an additional 7 min. NanoLuc luminescence was measured on SpectraMax M5 plate reader.

Screening for Single Enhancer Variants that Outperforms the Human SERPINA1 Enhancer

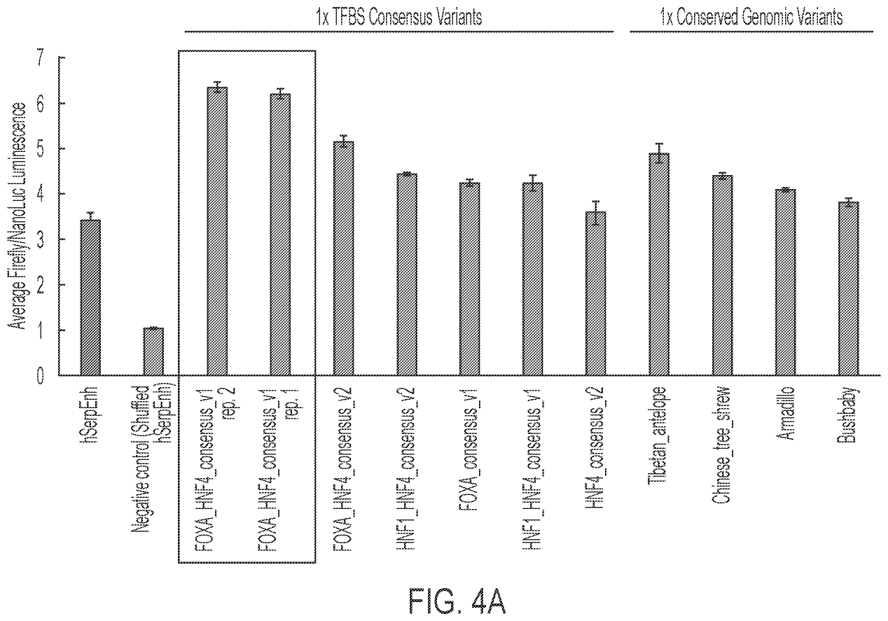

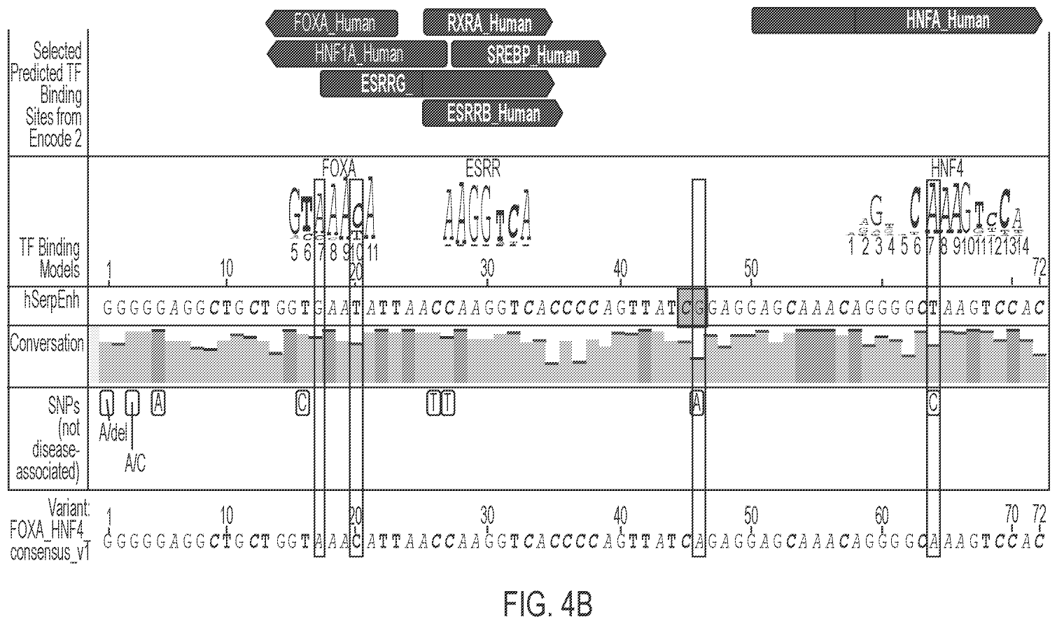

In a first round of screening, 30 variants including 20 conserved sequences from other organisms and 10 TFBS consensus variants which were placed in a plasmid were tested in vitro using the luciferase reporter assay as described herein. Data from the top 11 constructs are shown in FIG. 4A. FIG. 4A depicts results of the top 11 constructs in a screen of 30 single (1×) variants using luciferase reporter assay (n=3). Results are grouped by rationally designed enhancer variants (1×TFBS Consensus Variants) or conserved SERPINA1 enhancer regions identified in other species (1× Conserved Genomic Variants). The human SERPINA1 enhancer is shown far left. Error bars represent standard deviation. As shown in FIG. 4A, in a plasmid, hSerpEnh_FOXA_HNF4_consensus_v1, performed almost 2 times better than the benchmark hSerpEnh.

FIG. 4B depicts the sequence design of the top variant in this screen, hSerpEnh_FOXA_HNF4_consensus_v1. The top variant was designed by modifying the FOXA and HNF4 motifs identified in the human SERPINA1 enhancer to match their respective consensus sequences (GTGAATA to GTAAACA for FOXA and CTAAGT to CAAACT for HNF4). The internal CpG was ablated by changing the G, which both has lower sequence conservation than the C and is at the position of a human SNP, to an A to match the SNP. The FOXA and HNF4 binding sites were modified to match the consensus.

### Example 3: In Vitro Activity of Multimerized Human SERPINA1 Enhancer Variants

Selected multimerized enhancer variants from Table 4 were screened using the luciferase reporter assay (described in Example 2) to identify variants with enhanced performance compared to the human SERPINA1 enhancer.

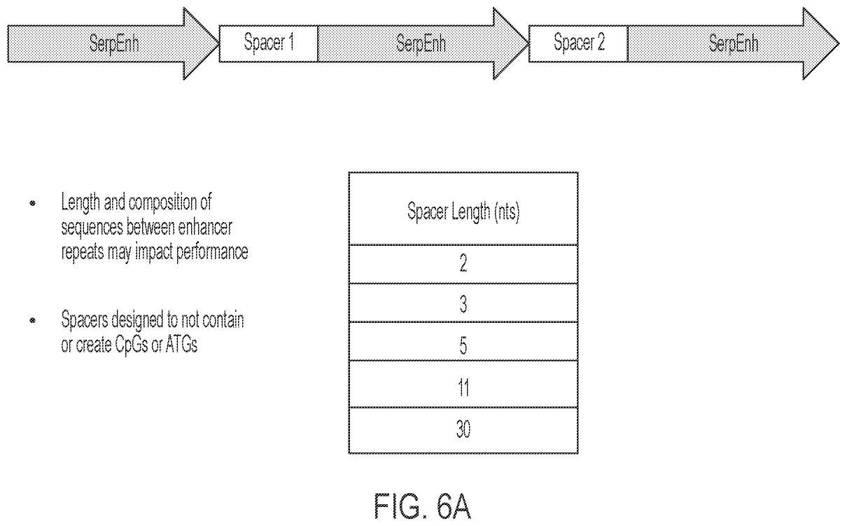

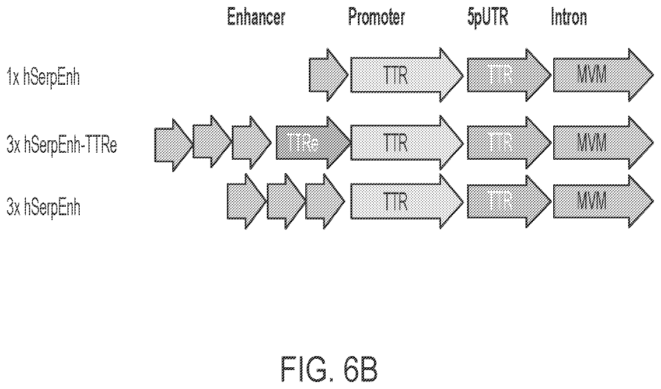

In a screen of 10 variants, the 1× version of hSerpEnh_FOXA_HNF4_consensus_v1 performed similarly to 3× hSerpEnh. The 3× version of hSerpEnh_FOXA_HNF4_consensus_v1 performed 1.6 times better than the 3× version of hSerpEnh and 3 times better than a single hSerpEnh (see FIG. 6). FIG. 5 depicts results of the screen of 10 multimerzied variants using a luciferase reporter assay (n=3). Results are grouped by 3× repeats of rationally designed enhancer variants (3× TFBS Variants), 3× repeats of conserved SERPINA1 enhancer regions identified in other species (3× Conserved Variant), 3× repeats of the human SERPINA1 enhancer separated by spacers of varying lengths and sequences (3× hSerpEnh Spacer Variants), and enhancers with varying numbers of repeats (#Repeat Variants). Results for the wild-type human SERPINA1 enhancer are shown in red. The comparison between the 3× human SERPINA1 enhancer variant and the 3× top performing variant is boxed. Two sets of technical triplicates were performed for the 1× and 3× human enhancers and the top performing 3× variant (r1, r2). Error bars represent standard deviation.

### Number of Enhancer Repeats and Length of Spacers

Like above, enhancers are often combined in series (multimerized/repeated enhancer sequences) to drive higher levels of transcription initiation. However, the principals underlying optimal number of repeats and orientation of enhancer regions are not well understood. Spacing between each iteration of repeated enhancers was hypothesized to be an attribute that impacts function, especially considering that DNA is a helix such that number of nucleotides between binding sites also changes their rotational spatial orientation. Spacers of different length between enhancers were tested. The length and sequence of spacers between SERPINA1 enhancer variant repeats were modified to screen for sequences that improved enhancer function. Spacers of length 2, 3, 5, 11, and 30 were designed to prevent introduction of CpGs or ATGs that may create cryptic translation start sites. 11 nt and 30 nt spacers that contain consensus FOXA and HNF4 binding sites were also designed and tested. (see, e.g., FIG. 6A).

A range of enhancer combinations for improved function, including various multimer enhancers and nucleotide spacer content, were tested in a dual luciferase transient transfection assay. Three main configurations as shown in FIG. 6B were tested: a single human Serpin enhancer (1× hSerpEnh), a 3× human Serpin enhancer (3× hSerpEnh) with spacers between the enhancer repeats and the transthyretin gene enhancer (TTRe) (3× hSerpEnh-TTRe), and multiple enhancers with spacers between enhancer repeats (e.g., 3×, 5× or 10× hSerpEnh). Variants with multiple enhancers for screening are shown in Table 4, above.

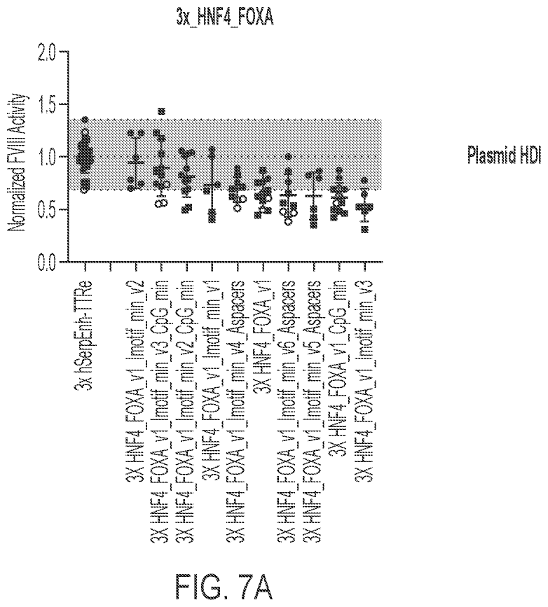

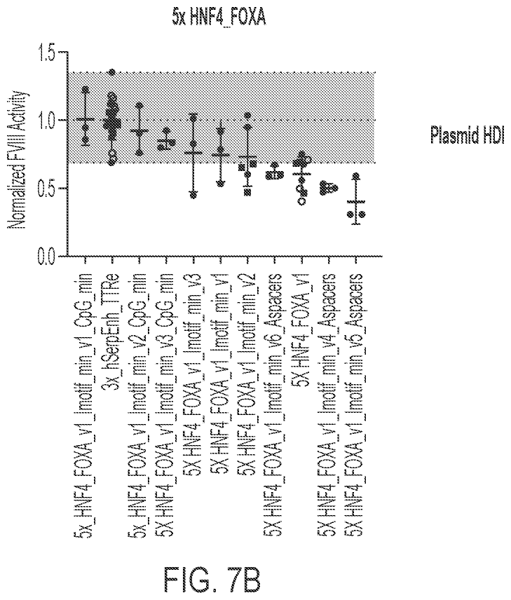

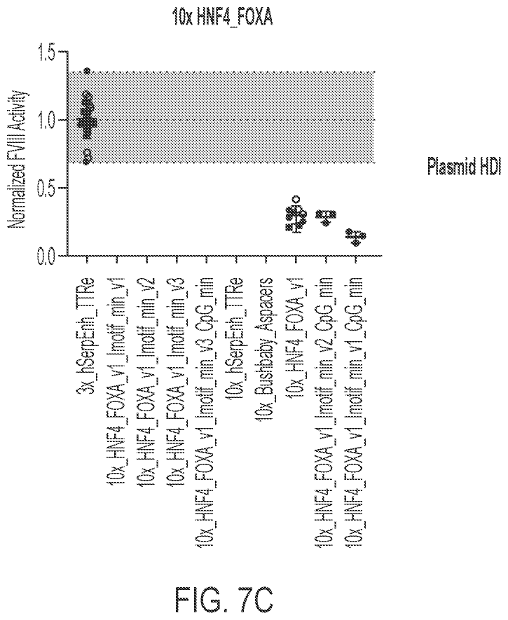

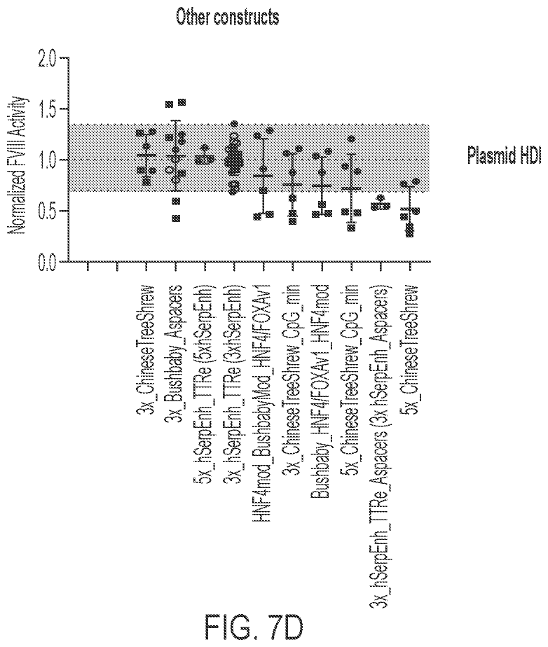

To determine the optimal number of repeats, HNF4_FOXA_v1, the top variant from the enhancer screen (FOXA_HNF4_consensus_v1), was placed in an array of 3, 5 or 10 repeats (e.g., 3× HNF4 FOXA v1; 5×HNF4 FOXA v1; and 10× HNF4 FOXA v1) to drive expression of FVIII from a plasmid. One dose of 50 ng plasmid containing FVIII ceDNA sequence was transfected into HepG2 cells. As shown in FIG. 7A, 7B, 3× and 5× variants performed better than HNF4 FOXA v1 variants repeated 10 times (10×) which did not exhibit a meaningful level of FVIII (see, e.g., FIG. 7C), suggesting that the Serpin enhancer exhibits superior performances when it is repeated in certain number, e.g., 3× to 5×, but not when it is repeated in an excessive number (e.g., 10×). A consistent observation was made with other Serpin Enhancer elements including, for example, that of bushbaby Serpin enhancer, Chinese tree shrew Serpin enhancer, and human Serpin enhancer (hSerpEnh)(FIG. 7D). The FVIII open reading frame (ORF) sequence used here was b-domain deleted codon optimized sequence as set forth in SEQ ID NO: 143 (hFVIII-F309S-BD226seq124-BDD-F309). Exemplary DNA constructs containing SEQ ID NO: 143 and enhancers/promoters of the present disclosure are shown in FIGS. 11 and 12.

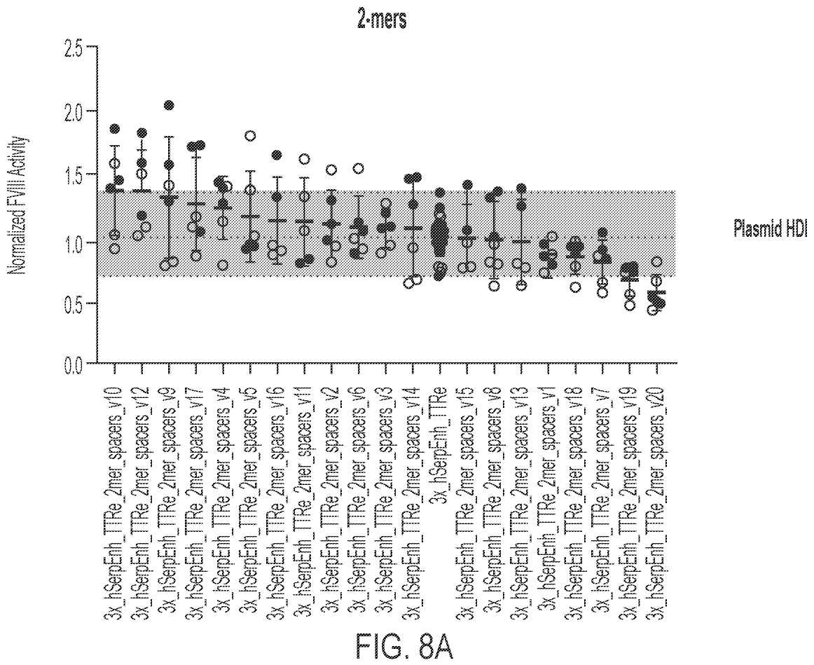

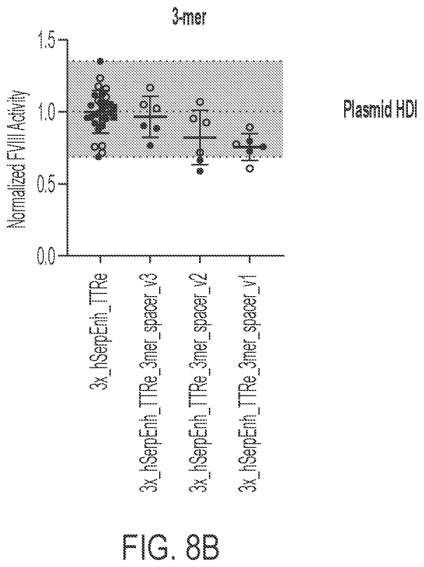

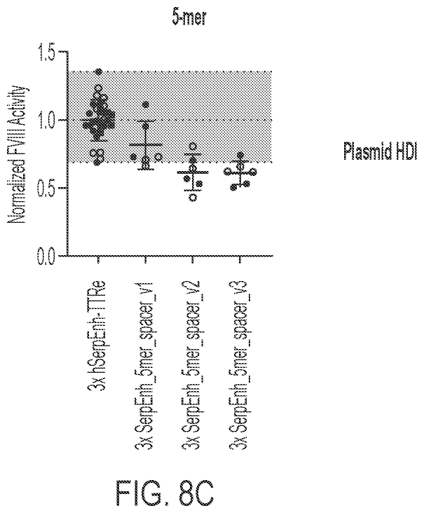

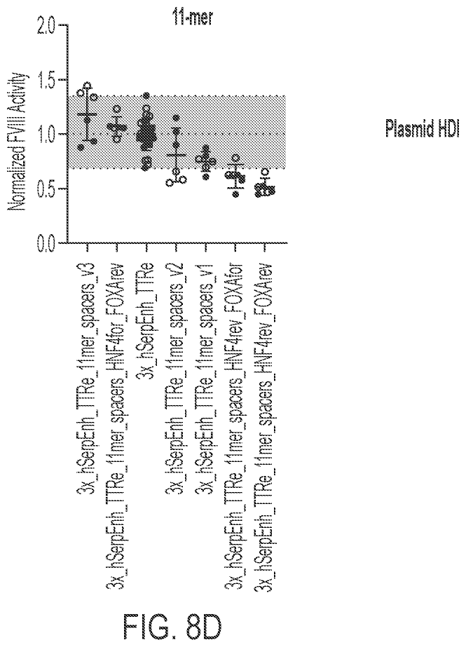

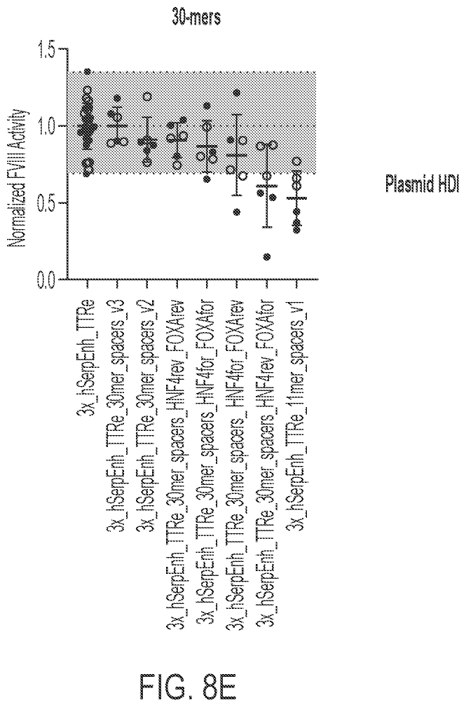

Further, spacers having difference number of nucleotides and sequence were tested to determine whether these repeated enhancer elements are sensitive to their spatial orientation created by a spacer as well as characteristics of the spacer created by spacer sequences. The length and sequence of spacers between hSERPINA1 enhancer repeats were modified to screen for sequences that improved enhancer function. As shown in FIGS. 8A-E, the hSerpEnh elements were sensitive to length as well as sequences of the spancers placed. In plasmid-mediated FVIII expression in HepG2 cells,) two nucleotide spacers generally exhibited improved activity as compared to 3× human Serpin enhancer with a single nucleotide spacer (3× hSerpinEnh-TTRe with “C” spacer). A certain level of spacer-sequence driven dependency was also observed as the activity seen with constructs having the similar length of spacer exhibited widely expression different profile depending on the DNA sequence (see 11-mer in FIG. 8D). Surprisingly, one spacer having 11 nucleotides performed exceedingly better than other 11-mers or other length spacers like three nucleotide spacers (see FIG. 8B) or 5 nucleotide spacers (see FIG. 8C). This spacer (version 3) was one of variants of 11-mer spacers having the sequence of “TGCAAAGTCCT” (SEQ ID NO: 144) and/or “AGTGTTTACAA” (SEQ ID NO: 145) as shown in SEQ ID NO:71.

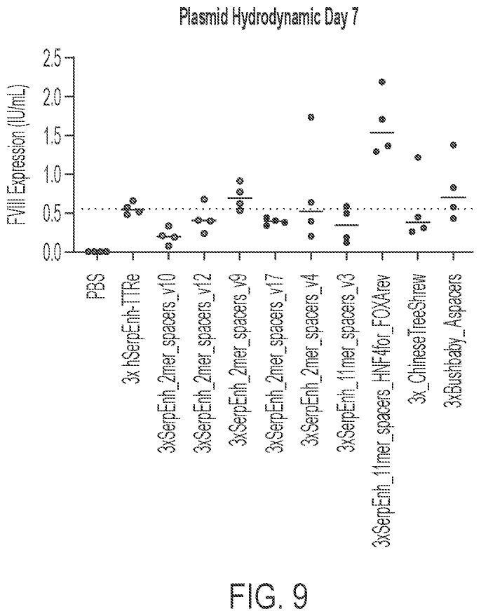

To determine whether Serpin enhancer variants (SerpEnhs derived from Bushbaby or Chinese Tree Shrew), plasmid FVIII constructs containing 3× Bushbaby SerpEnh having adenine (A) spacers or 3× Chinese Tree Shrew were injucted hydrodynamically into Rag 2 mice to drive expression of FVIII (HDI tail vein injection of 50 ng plasmid containing FVIII ceDNA sequence on day 0 with a single blood collection at day 7 for the measurement of FVIII activity). Surprisingly, the 3× Serpin A enhancer sequence derived from Bushbaby having a single nucleotide (adenine) as spacers exhibited increased FVIII expression (FIG. 9). Consistent with the observations above, 3× human Serpin enhancer sequence with 11 mer spacers also exhibited increased expression of FVIII as compared to 3× hSerpEhn (FIG. 9). It was also noted that 3× Chinese Tree Shrew enhancers that has 3 missing nucleotides in its 5′ end as compared to the human SerpEnh sequence (see FIG. 1) also exhibited an equivalent level of expression as compared to those of various 3× human serpin enhancer constructs (FIG. 9).

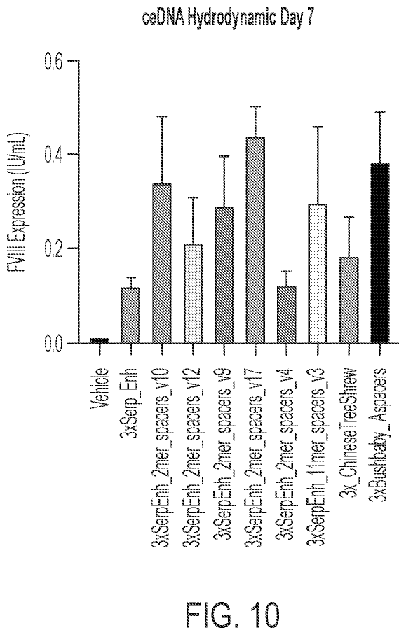

To determine whether the capacity of these enhancers could vary in a platform-dependent manner (e.g., plasmid v. closed-ended DNA), corresponding ceDNA vectors representative of the experimental results shown in FIGS. 7-9 were prepared and hydrodynamically injected into Rag2 mice as described above. FIG. 10 shows the result of FVIII expression from spacer variants of hSerpEnh (2mers and 11 mers) and Serpin enhancer variants (3× bushbaby Serpin enahancer and 3× Chinese tree shrew Serpin enhancer). Surprisingly, in the ceDNA platform, all of the SerpEnh variants tested (i.e., 3× Bushbaby SerpEnh variant, 3× Chinese Tree shrew SerpEnh variant, 11 mer spacer variants and 2mer spacer variants) exhibited equivalent or superior FVIII expression profiles as compared to that of 3× hSerpEnh, suggesting that these enhancers can be successfully implemented to drive expression of a therapeutic protein like FVIII in vivo.

The following nucleotide sequence is a ceDNA-plasmid sequence comprising a left ITR: spacer: bushbaby serpin enhancer (3× Bushbaby_Aspacers): TTRe (TTR enhancer): TTR liver-specific promoter: MVM intron: B-domain deleted FVIII: WPRE 3′UTR: bGH: spacer right ITR: right ITR. Detailed annotations for this construct are shown in FIG. 11.

(SEQ ID NO: 146)
AAAGTAGCCGAAGATGACGGTTTGTCACATGGAGTTGGCAGGATGTTTGATTAAAAACATAACAGGAA
GAAAAATGCCCCGCTGTGGGCGGACAAAATAGTTGGGAACTGGGAGGGGTGGAAATGGAGTTTTTAAG
GATTATTTAGGGAAGAGTGACAAAATAGATGGGAACTGGGTGTAGCGTCGTAAGCTAATACGAAAATT
AAAAATGACAAAATAGTTTGGAACTAGATTTCACTTATCTGGTTCGGATCTCCTAGGCCTGCAGGCAG
CTGCGCGCTCGCTCGCTCACTGAGGCCGCCCGGGCAAAGCCCGGGCGTCGGGCGACCTTTGGTCGCCC
GGCCTCAGTGAGCGAGCGAGCGCGCAGAGAGGGAGTGGCCAACTCCATCACTAGGGGTTCCTTGTAGT
TAATGATTAACCCGCCATGCTACTTATCGCGGCCGCAGGGGAAGCTACTGGTGAATATTAACCAAGGT
CACCCAGTTATCAGGGAGCAAACAGGAGCTAAGTCCATAGGGGGAAGCTACTGGTGAATATTAACCAA
GGTCACCCAGTTATCAGGGAGCAAACAGGAGCTAAGTCCATAGGGGGAAGCTACTGGTGAATATTAAC
CAAGGTCACCCAGTTATCAGGGAGCAAACAGGAGCTAAGTCCATGGTACCCACTGGGAGGATGTTGAG
TAAGATGGAAAACTACTGATGACCCTTGCAGAGACAGAGTATTAGGACATGTTTGAACAGGGGCCGGG
CGATCAGCAGGTAGCTCTAGAGGATCCCCGTCTGTCTGCACATTTCGTAGAGCGAGTGTTCCGATACT
CTAATCTCCCTAGGCAAGGTTCATATTTGTGTAGGTTACTTATTCTCCTTTTGTTGACTAAGTCAATA
ATCAGAATCAGCAGGTTTGGAGTCAGCTTGGCAGGGATCAGCAGCCTGGGTTGGAAGGAGGGGGTATA
AAAGCCCCTTCACCAGGAGAAGCCGTCACACAGATCCACAAGCTCCTGAAGAGGTAAGGGTTTAAGGG
ATGGTTGGTTGGTGGGGTATTAATGTTTAATTACCTGGAGCACCTGCCTGAAATCACTTTTTTTCAGG
TTGGTTTAAACGCCGCCACCATGCAGATTGAGCTGAGCACCTGCTTCTTCCTGTGCCTGCTGAGGTTC
TGCTTCTCTGCCACCAGGAGATACTACCTGGGGGCTGTGGAGCTGAGCTGGGACTACATGCAGTCTGA
CCTGGGGGAGCTGCCTGTGGATGCCAGGTTCCCCCCCAGAGTGCCCAAGAGCTTCCCCTTCAACACCT
CTGTGGTGTACAAGAAGACCCTGTTTGTGGAGTTCACTGACCACCTGTTCAACATTGCCAAGCCCAGG
CCCCCCTGGATGGGCCTGCTGGGCCCCACCATCCAGGCTGAGGTGTATGACACTGTGGTGATCACCCT
GAAGAACATGGCCAGCCACCCTGTGAGCCTGCATGCTGTGGGGGTGAGCTACTGGAAGGCCTCTGAGG
GGGCTGAGTATGATGACCAGACCAGCCAGAGGGAGAAGGAGGATGACAAGGTGTTCCCTGGGGGCAGC
CACACCTATGTGTGGCAGGTGCTGAAGGAGAATGGCCCCATGGCCTCTGACCCCCTGTGCCTGACCTA
CAGCTACCTGAGCCATGTGGACCTGGTGAAGGACCTGAACTCTGGCCTGATTGGGGCCCTGCTGGTGT
GCAGGGAGGGCAGCCTGGCCAAGGAGAAGACCCAGACCCTGCACAAGTTCATCCTGCTGTTTGCTGTG
TTTGATGAGGGCAAGAGCTGGCACTCTGAAACCAAGAACAGCCTGATGCAGGACAGGGATGCTGCCTC
TGCCAGGGCCTGGCCCAAGATGCACACTGTGAATGGCTATGTGAACAGGAGCCTGCCTGGCCTGATTG
GCTGCCACAGGAAGTCTGTGTACTGGCATGTGATTGGCATGGGCACCACCCCTGAGGTGCACAGCATC
TTCCTGGAGGGCCACACCTTCCTGGTCAGGAACCACAGGCAGGCCAGCCTGGAGATCAGCCCCATCAC
CTTCCTGACTGCCCAGACCCTGCTGATGGACCTGGGCCAGTTCCTGCTGTTCTGCCACATCAGCAGCC
ACCAGCATGATGGCATGGAGGCCTATGTGAAGGTGGACAGCTGCCCTGAGGAGCCCCAGCTGAGGATG
AAGAACAATGAGGAGGCTGAGGACTATGATGATGACCTGACTGACTCTGAGATGGATGTGGTGAGGTT
TGATGATGACAACAGCCCCAGCTTCATCCAGATCAGGTCTGTGGCCAAGAAGCACCCCAAGACCTGGG
TGCACTACATTGCTGCTGAGGAGGAGGACTGGGACTATGCCCCCCTGGTGCTGGCCCCTGATGACAGG
AGCTACAAGAGCCAGTACCTGAACAATGGCCCCCAGAGGATTGGCAGGAAGTACAAGAAGGTCAGGTT
CATGGCCTACACTGATGAAACCTTCAAGACCAGGGAGGCCATCCAGCATGAGTCTGGCATCCTGGGCC
CCCTGCTGTATGGGGAGGTGGGGGACACCCTGCTGATCATCTTCAAGAACCAGGCCAGCAGGCCCTAC
AACATCTACCCCCATGGCATCACTGATGTGAGGCCCCTGTACAGCAGGAGGCTGCCCAAGGGGGTGAA
GCACCTGAAGGACTTCCCCATCCTGCCTGGGGAGATCTTCAAGTACAAGTGGACTGTGACTGTGGAGG
ATGGCCCCACCAAGTCTGACCCCAGGTGCCTGACCAGATACTACAGCAGCTTTGTGAACATGGAGAGG
GACCTGGCCTCTGGCCTGATTGGCCCCCTGCTGATCTGCTACAAGGAGTCTGTGGACCAGAGGGGCAA
CCAGATCATGTCTGACAAGAGGAATGTGATCCTGTTCTCTGTGTTTGATGAGAACAGGAGCTGGTACC
TGACTGAGAACATCCAGAGGTTCCTGCCCAACCCTGCTGGGGTGCAGCTGGAGGACCCTGAGTTCCAG
GCCAGCAACATCATGCACAGCATCAATGGCTATGTGTTTGACAGCCTGCAGCTGTCTGTGTGCCTGCA
TGAGGTGGCCTACTGGTACATCCTGAGCATTGGGGCCCAGACTGACTTCCTGTCTGTGTTCTTCTCTG
GCTACACCTTCAAGCACAAGATGGTGTATGAGGACACCCTGACCCTGTTCCCCTTCTCTGGGGAGACT
GTGTTCATGAGCATGGAGAACCCTGGCCTGTGGATTCTGGGCTGCCACAACTCTGACTTCAGGAACAG
GGGCATGACTGCCCTGCTGAAAGTCTCCAGCTGTGACAAGAACACTGGGGACTACTATGAGGACAGCT
ATGAGGACATCTCTGCCTACCTGCTGAGCAAGAACAATGCCATTGAGCCCAGGAGCTTCAGCCAGAAT
AGCAGGCACCCCAGCACCAGGCAGAAGCAGTTCAATGCCACCACCATCCCAGAGAATACCACCCTGCA
GTCTGACCAGGAGGAGATTGACTATGATGACACCATCTCTGTGGAGATGAAGAAGGAGGACTTTGACA
TCTACGACGAGGACGAGAACCAGAGCCCCAGGAGCTTCCAGAAGAAGACCAGGCACTACTTCATTGCT
GCTGTGGAGAGGCTGTGGGACTATGGCATGAGCAGCAGCCCCCATGTGCTGAGGAACAGGGCCCAGTC
TGGCTCTGTGCCCCAGTTCAAGAAGGTGGTGTTCCAGGAGTTCACTGATGGCAGCTTCACCCAGCCCC
TGTACAGAGGGGAGCTGAATGAGCACCTGGGCCTGCTGGGCCCCTACATCAGGGCTGAGGTGGAGGAC
AACATCATGGTGACCTTCAGGAACCAGGCCAGCAGGCCCTACAGCTTCTACAGCAGCCTGATCAGCTA
TGAGGAGGACCAGAGGCAGGGGGCTGAGCCCAGGAAGAACTTTGTGAAGCCCAATGAAACCAAGACCT
ACTTCTGGAAGGTGCAGCACCACATGGCCCCCACCAAGGATGAGTTTGACTGCAAGGCCTGGGCCTAC
TTCTCTGATGTGGACCTGGAGAAGGATGTGCACTCTGGCCTGATTGGCCCCCTGCTGGTGTGCCACAC
CAACACCCTGAACCCTGCCCATGGCAGGCAGGTGACTGTGCAGGAGTTTGCCCTGTTCTTCACCATCT
TTGATGAAACCAAGAGCTGGTACTTCACTGAGAACATGGAGAGGAACTGCAGGGCCCCCTGCAACATC
CAGATGGAGGACCCCACCTTCAAGGAGAACTACAGGTTCCATGCCATCAATGGCTACATCATGGACAC
CCTGCCTGGCCTGGTGATGGCCCAGGACCAGAGGATCAGGTGGTACCTGCTGAGCATGGGCAGCAATG
AGAACATCCACAGCATCCACTTCTCTGGCCATGTGTTCACTGTGAGGAAGAAGGAGGAGTACAAGATG
GCCCTGTACAACCTGTACCCTGGGGTGTTTGAGACTGTGGAGATGCTGCCCAGCAAGGCTGGCATCTG
GAGGGTGGAGTGCCTGATTGGGGAGCACCTGCATGCTGGCATGAGCACCCTGTTCCTGGTGTACAGCA
ACAAGTGCCAGACCCCCCTGGGCATGGCCTCTGGCCACATCAGGGACTTCCAGATCACTGCCTCTGGC
CAGTATGGCCAGTGGGCCCCCAAGCTGGCCAGGCTGCACTACTCTGGCAGCATCAATGCCTGGAGCAC
CAAGGAGCCCTTCAGCTGGATCAAGGTGGACCTGCTGGCCCCCATGATCATCCATGGCATCAAGACCC
AGGGGGCCAGGCAGAAGTTCAGCAGCCTGTACATCAGCCAGTTCATCATCATGTACAGCCTGGATGGC
AAGAAGTGGCAGACCTACAGGGGCAACAGCACTGGCACCCTGATGGTGTTCTTTGGCAATGTGGACAG
CTCTGGCATCAAGCACAACATCTTCAACCCCCCCATCATTGCCAGATACATCAGGCTGCACCCCACCC
ACTACAGCATCAGGAGCACCCTGAGGATGGAGCTGATGGGCTGTGACCTGAACAGCTGCAGCATGCCC
CTGGGCATGGAGAGCAAGGCCATCTCTGATGCCCAGATCACTGCCAGCAGCTACTTCACCAACATGTT
TGCCACCTGGAGCCCCAGCAAGGCCAGGCTGCACCTGCAGGGCAGGAGCAATGCCTGGAGGCCCCAGG
TCAACAACCCCAAGGAGTGGCTGCAGGTGGACTTCCAGAAGACCATGAAGGTGACTGGGGTGACCACC
CAGGGGGTGAAGAGCCTGCTGACCAGCATGTATGTGAAGGAGTTCCTGATCAGCAGCAGCCAGGATGG
CCACCAGTGGACCCTGTTCTTCCAGAATGGCAAGGTGAAGGTGTTCCAGGGCAACCAGGACAGCTTCA
CCCCTGTGGTGAACAGCCTGGACCCCCCCCTGCTGACCAGATACCTGAGGATTCACCCCCAGAGCTGG
GTGCACCAGATTGCCCTGAGGATGGAGGTGCTGGGCTGTGAGGCCCAGGACCTGTACTGATTAATTAA
GAGCATCTTACCGCCATTTATTCCCATATTTGTTCTGTTTTTCTTGATTTGGGTATACATTTAAATGT
TAATAAAACAAAATGGTGGGGCAATCATTTACATTTTTAGGGATATGTAATTACTAGTTCAGGTGTAT
TGCCACAAGACAAACATGTTAAGAAACTTTCCCGTTATTTACGCTCTGTTCCTGTTAATCAACCTCTG
GATTACAAAATTTGTGAAAGATTGACTGATATTCTTAACTATGTTGCTCCTTTTACGCTGTGTGGATA
TGCTGCTTTATAGCCTCTGTATCTAGCTATTGCTTCCCGTACGGCTTTCGTTTTCTCCTCCTTGTATA
AATCCTGGTTGCTGTCTCTTTTAGAGGAGTTGTGGCCCGTTGTCCGTCAACGTGGCGTGGTGTGCTCT
GTGTTTGCTGACGCAACCCCCACTGGCTGGGGCATTGCCACCACCTGTCAACTCCTTTCTGGGACTTT
CGCTTTCCCCCTCCCGATCGCCACGGCAGAACTCATCGCCGCCTGCCTTGCCCGCTGCTGGACAGGGG
CTAGGTTGCTGGGCACTGATAATTCCGTGGTGTTGTCTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATGGCTCTAGAGCAT
GGCTACGTAGATAAGTAGCATGGCGGGTTAATCATTAACTACACCTGCAGGAGGAACCCCTAGTGATG
GAGTTGGCCACTCCCTCTCTGCGCGCTCGCTCGCTCACTGAGGCCGGGCGACCAAAGGTCGCCCGACG
CCCGGGCGGCCTCAGTGAGCGAGCGAGCGCGCAGCTGCCTGCAGGGGCGCGCCTCGAGCCATGGTGCT
AGCAGCTGATGCATAGCATGCGGTACCGGGAGATGGGGGAGGCTAACTGAAACACGGAAGGAGACAAT
ACCGGAAGGAACCCGCGCTATGACGGCAATAAAAAGACAGAATAAAACGCACGGGTGTTGGGTCGTTT
GTTCATAAACGCGGGGTTCGGTCCCAGGGCTGGCACTCTGTCGATACCCCACCGAGACCCCATTGGGA
CCAATACGCCCGCGTTTCTTCCTTTTCCCCACCCCAACCCCCAAGTTCGGGTGAAGGCCCAGGGCTCG
CAGCCAACGTCGGGGCGGCAAGCCCTGCCATAGCCACTACGGGTACGTAGGCCAACCACTAGAACTAT
AGCTAGAGTCCTGGGCGAACAAACGATGCTCGCCTTCCAGAAAACCGAGGATGCGAACCACTTCATCC
GGGGTCAGCACCACCGGCAAGCGCCGCGACGGCCGAGGTCTACCGATCTCCTGAAGCCAGGGCAGATC
CGTGCACAGCACCTTGCCGTAGAAGAACAGCAAGGCCGCCAATGCCTGACGATGCGTGGAGACCGAAA
CCTTGCGCTCGTTCGCCAGCCAGGACAGAAATGCCTCGACTTCGCTGCTGCCCAAGGTTGCCGGGTGA
CGCACACCGTGGAAACGGATGAAGGCACGAACCCAGTTGACATAAGCCTGTTCGGTTCGTAAACTGTA
ATGCAAGTAGCGTATGCGCTCACGCAACTGGTCCAGAACCTTGACCGAACGCAGCGGTGGTAACGGCG
CAGTGGCGGTTTTCATGGCTTGTTATGACTGTTTTTTTGTACAGTCTATGCCTCGGGCATCCAAGCAG
CAAGCGCGTTACGCCGTGGGTCGATGTTTGATGTTATGGAGCAGCAACGATGTTACGCAGCAGCAACG
ATGTTACGCAGCAGGGCAGTCGCCCTAAAACAAAGTTAGGTGGCTCAAGTATGGGCATCATTCGCACA
TGTAGGCTCGGCCCTGACCAAGTCAAATCCATGCGGGCTGCTCTTGATCTTTTCGGTCGTGAGTTCGG
AGACGTAGCCACCTACTCCCAACATCAGCCGGACTCCGATTACCTCGGGAACTTGCTCCGTAGTAAGA
CATTCATCGCGCTTGCTGCCTTCGACCAAGAAGCGGTTGTTGGCGCTCTCGCGGCTTACGTTCTGCCC
AGGTTTGAGCAGCCGCGTAGTGAGATCTATATCTATGATCTCGCAGTCTCCGGCGAGCACCGGAGGCA
GGGCATTGCCACCGCGCTCATCAATCTCCTCAAGCATGAGGCCAACGCGCTTGGTGCTTATGTGATCT
ACGTGCAAGCAGATTACGGTGACGATCCCGCAGTGGCTCTCTATACAAAGTTGGGCATACGGGAAGAA
GTGATGCACTTTGATATCGACCCAAGTACCGCCACCTAACAATTCGTTCAAGCCGAGATCGGCTTCCC
GGCCGCGGAGTTGTTCGGTAAATTGTCACAACGCCGCGAATATAGTCTTTACCATGCCCTTGGCCACG
CCCCTCTTTAATACGACGGGCAATTTGCACTTCAGAAAATGAAGAGTTTGCTTTAGCCATAACAAAAG
TCCAGTATGCTTTTTCACAGCATAACTGGACTGATTTCAGTTTACAACTATTCTGTCTAGTTTAAGAC
TTTATTGTCATAGTTTAGATCTATTTTGTTCAGTTTAAGACTTTATTGTCCGCCCACACCCGCTTACG
CAGGGCATCCATTTATTACTCAACCGTAACCGATTTTGCCAGGTTACGCGGCTGGTCTGCGGTGTGAA
ATACCGCACAGATGCGTAAGGAGAAAATACCGCATCAGGCGCTCTTCCGCTTCCTCGCTCACTGACTC
GCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAAATCGACGCTC
AAGTCAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAGCGTG
GCGCTTTCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGGACAGTATTTGGTAT
CTGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGAAATTGTAAACGTTAATATTTTGTTAAAATTCGCGTTAAATTTTTGTTAAATCAGCTCATTTTTT
AACCAATAGGCCGAAATCGGCAAAATCCCTTATAAATCAAAAGAATAGACCGAGATAGGGTTGAGTGT
TGTTCCAGTTTGGAACAAGAGTCCACTATTAAAGAACGTGGACTCCAACGTCAAAGGGCGAAAAACCG
TCTATCAGGGCGATGGCCCACTACGTGAACCATCACCCTAATCAAGTTTTTTGGGGTCGAGGTGCCGT
AAAGCACTAAATCGGAACCCTAAAGGGAGCCCCCGATTTAGAGCTTGACGGGGAAAGCCGGCGAACGT
GGCGAGAAAGGAAGGGAAGAAAGCGAAAGGAGCGGGCGCTAGGGCGCTGGCAAGTGTAGCGGTCACGC
TGCGCGTAACCACCACACCCGCCGCGCTTAATGCGCCGCTACAGGGCGCGTCCCATTCGCCATTCAGG
CTGCAAATAAGCGTTGATATTCAGTCAATTACAAACATTAATAACGAAGAGATGACAGAAAAATTTTC
ATTCTGTGACAGAGAA

The following nucleotide sequence is a ceDNA-plasmid sequence comprising a left ITR: spacer: 3× hSerpEnh: TTRe (TTR enhancer): TTR liver-specific promoter: MVM intron: B-domain deleted FVIII: WPRE 3′UTR: bGH: spacer right ITR: right ITR. Detailed annotations for this ceDNA vector are shown in FIG. 12.

(SEQ ID NO: 147)
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTTGTAGTTAATGATTAACCCGCCATGCTACTTATCGCGGCCGCGGGGGAGGCTGCTGGTGAATA
TTAACCAAGGTCACCCCAGTTATCGGAGGAGCAAACAGGGGCTAAGTCCACCGGGGGAGGCTGCTGGT
GAATATTAACCAAGGTCACCCCAGTTATCGGAGGAGCAAACAGGGGCTAAGTCCACCGGGGGAGGCTG
CTGGTGAATATTAACCAAGGTCACCCCAGTTATCGGAGGAGCAAACAGGGGCTAAGTCCACGGTACCC
ACTGGGAGGATGTTGAGTAAGATGGAAAACTACTGATGACCCTTGCAGAGACAGAGTATTAGGACATG
TTTGAACAGGGGCCGGGCGATCAGCAGGTAGCTCTAGAGGATCCCCGTCTGTCTGCACATTTCGTAGA
GCGAGTGTTCCGATACTCTAATCTCCCTAGGCAAGGTTCATATTTGTGTAGGTTACTTATTCTCCTTT
TGTTGACTAAGTCAATAATCAGAATCAGCAGGTTTGGAGTCAGCTTGGCAGGGATCAGCAGCCTGGGT
TGGAAGGAGGGGGTATAAAAGCCCCTTCACCAGGAGAAGCCGTCACACAGATCCACAAGCTCCTGAAG
AGGTAAGGGTTTAAGGGATGGTTGGTTGGTGGGGTATTAATGTTTAATTACCTGGAGCACCTGCCTGA
AATCACTTTTTTTCAGGTTGGTTTAAACGCCGCCACCATGCAGATTGAGCTGAGCACCTGCTTCTTCC
TGTGCCTGCTGAGGTTCTGCTTCTCTGCCACCAGGAGATACTACCTGGGGGCTGTGGAGCTGAGCTGG
GACTACATGCAGTCTGACCTGGGGGAGCTGCCTGTGGATGCCAGGTTCCCCCCCAGAGTGCCCAAGAG
CTTCCCCTTCAACACCTCTGTGGTGTACAAGAAGACCCTGTTTGTGGAGTTCACTGACCACCTGTTCA
ACATTGCCAAGCCCAGGCCCCCCTGGATGGGCCTGCTGGGCCCCACCATCCAGGCTGAGGTGTATGAC
ACTGTGGTGATCACCCTGAAGAACATGGCCAGCCACCCTGTGAGCCTGCATGCTGTGGGGGTGAGCTA
CTGGAAGGCCTCTGAGGGGGCTGAGTATGATGACCAGACCAGCCAGAGGGAGAAGGAGGATGACAAGG
TGTTCCCTGGGGGCAGCCACACCTATGTGTGGCAGGTGCTGAAGGAGAATGGCCCCATGGCCTCTGAC
CCCCTGTGCCTGACCTACAGCTACCTGAGCCATGTGGACCTGGTGAAGGACCTGAACTCTGGCCTGAT
TGGGGCCCTGCTGGTGTGCAGGGAGGGCAGCCTGGCCAAGGAGAAGACCCAGACCCTGCACAAGTTCA
TCCTGCTGTTTGCTGTGTTTGATGAGGGCAAGAGCTGGCACTCTGAAACCAAGAACAGCCTGATGCAG
GACAGGGATGCTGCCTCTGCCAGGGCCTGGCCCAAGATGCACACTGTGAATGGCTATGTGAACAGGAG
CCTGCCTGGCCTGATTGGCTGCCACAGGAAGTCTGTGTACTGGCATGTGATTGGCATGGGCACCACCC
CTGAGGTGCACAGCATCTTCCTGGAGGGCCACACCTTCCTGGTCAGGAACCACAGGCAGGCCAGCCTG
GAGATCAGCCCCATCACCTTCCTGACTGCCCAGACCCTGCTGATGGACCTGGGCCAGTTCCTGCTGTT
CTGCCACATCAGCAGCCACCAGCATGATGGCATGGAGGCCTATGTGAAGGTGGACAGCTGCCCTGAGG
AGCCCCAGCTGAGGATGAAGAACAATGAGGAGGCTGAGGACTATGATGATGACCTGACTGACTCTGAG
ATGGATGTGGTGAGGTTTGATGATGACAACAGCCCCAGCTTCATCCAGATCAGGTCTGTGGCCAAGAA
GCACCCCAAGACCTGGGTGCACTACATTGCTGCTGAGGAGGAGGACTGGGACTATGCCCCCCTGGTGC
TGGCCCCTGATGACAGGAGCTACAAGAGCCAGTACCTGAACAATGGCCCCCAGAGGATTGGCAGGAAG
TACAAGAAGGTCAGGTTCATGGCCTACACTGATGAAACCTTCAAGACCAGGGAGGCCATCCAGCATGA
GTCTGGCATCCTGGGCCCCCTGCTGTATGGGGAGGTGGGGGACACCCTGCTGATCATCTTCAAGAACC
AGGCCAGCAGGCCCTACAACATCTACCCCCATGGCATCACTGATGTGAGGCCCCTGTACAGCAGGAGG
CTGCCCAAGGGGGTGAAGCACCTGAAGGACTTCCCCATCCTGCCTGGGGAGATCTTCAAGTACAAGTG
GACTGTGACTGTGGAGGATGGCCCCACCAAGTCTGACCCCAGGTGCCTGACCAGATACTACAGCAGCT
TTGTGAACATGGAGAGGGACCTGGCCTCTGGCCTGATTGGCCCCCTGCTGATCTGCTACAAGGAGTCT
GTGGACCAGAGGGGCAACCAGATCATGTCTGACAAGAGGAATGTGATCCTGTTCTCTGTGTTTGATGA
GAACAGGAGCTGGTACCTGACTGAGAACATCCAGAGGTTCCTGCCCAACCCTGCTGGGGTGCAGCTGG
AGGACCCTGAGTTCCAGGCCAGCAACATCATGCACAGCATCAATGGCTATGTGTTTGACAGCCTGCAG
CTGTCTGTGTGCCTGCATGAGGTGGCCTACTGGTACATCCTGAGCATTGGGGCCCAGACTGACTTCCT
GTCTGTGTTCTTCTCTGGCTACACCTTCAAGCACAAGATGGTGTATGAGGACACCCTGACCCTGTTCC
CCTTCTCTGGGGAGACTGTGTTCATGAGCATGGAGAACCCTGGCCTGTGGATTCTGGGCTGCCACAAC
TCTGACTTCAGGAACAGGGGCATGACTGCCCTGCTGAAAGTCTCCAGCTGTGACAAGAACACTGGGGA
CTACTATGAGGACAGCTATGAGGACATCTCTGCCTACCTGCTGAGCAAGAACAATGCCATTGAGCCCA
GGAGCTTCAGCCAGAATAGCAGGCACCCCAGCACCAGGCAGAAGCAGTTCAATGCCACCACCATCCCA
GAGAATACCACCCTGCAGTCTGACCAGGAGGAGATTGACTATGATGACACCATCTCTGTGGAGATGAA
GAAGGAGGACTTTGACATCTACGACGAGGACGAGAACCAGAGCCCCAGGAGCTTCCAGAAGAAGACCA
GGCACTACTTCATTGCTGCTGTGGAGAGGCTGTGGGACTATGGCATGAGCAGCAGCCCCCATGTGCTG
AGGAACAGGGCCCAGTCTGGCTCTGTGCCCCAGTTCAAGAAGGTGGTGTTCCAGGAGTTCACTGATGG
CAGCTTCACCCAGCCCCTGTACAGAGGGGAGCTGAATGAGCACCTGGGCCTGCTGGGCCCCTACATCA
GGGCTGAGGTGGAGGACAACATCATGGTGACCTTCAGGAACCAGGCCAGCAGGCCCTACAGCTTCTAC
AGCAGCCTGATCAGCTATGAGGAGGACCAGAGGCAGGGGGCTGAGCCCAGGAAGAACTTTGTGAAGCC
CAATGAAACCAAGACCTACTTCTGGAAGGTGCAGCACCACATGGCCCCCACCAAGGATGAGTTTGACT
GCAAGGCCTGGGCCTACTTCTCTGATGTGGACCTGGAGAAGGATGTGCACTCTGGCCTGATTGGCCCC
CTGCTGGTGTGCCACACCAACACCCTGAACCCTGCCCATGGCAGGCAGGTGACTGTGCAGGAGTTTGC
CCTGTTCTTCACCATCTTTGATGAAACCAAGAGCTGGTACTTCACTGAGAACATGGAGAGGAACTGCA
GGGCCCCCTGCAACATCCAGATGGAGGACCCCACCTTCAAGGAGAACTACAGGTTCCATGCCATCAAT
GGCTACATCATGGACACCCTGCCTGGCCTGGTGATGGCCCAGGACCAGAGGATCAGGTGGTACCTGCT
GAGCATGGGCAGCAATGAGAACATCCACAGCATCCACTTCTCTGGCCATGTGTTCACTGTGAGGAAGA
AGGAGGAGTACAAGATGGCCCTGTACAACCTGTACCCTGGGGTGTTTGAGACTGTGGAGATGCTGCCC
AGCAAGGCTGGCATCTGGAGGGTGGAGTGCCTGATTGGGGAGCACCTGCATGCTGGCATGAGCACCCT
GTTCCTGGTGTACAGCAACAAGTGCCAGACCCCCCTGGGCATGGCCTCTGGCCACATCAGGGACTTCC
AGATCACTGCCTCTGGCCAGTATGGCCAGTGGGCCCCCAAGCTGGCCAGGCTGCACTACTCTGGCAGC
ATCAATGCCTGGAGCACCAAGGAGCCCTTCAGCTGGATCAAGGTGGACCTGCTGGCCCCCATGATCAT
CCATGGCATCAAGACCCAGGGGGCCAGGCAGAAGTTCAGCAGCCTGTACATCAGCCAGTTCATCATCA
TGTACAGCCTGGATGGCAAGAAGTGGCAGACCTACAGGGGCAACAGCACTGGCACCCTGATGGTGTTC
TTTGGCAATGTGGACAGCTCTGGCATCAAGCACAACATCTTCAACCCCCCCATCATTGCCAGATACAT
CAGGCTGCACCCCACCCACTACAGCATCAGGAGCACCCTGAGGATGGAGCTGATGGGCTGTGACCTGA
ACAGCTGCAGCATGCCCCTGGGCATGGAGAGCAAGGCCATCTCTGATGCCCAGATCACTGCCAGCAGC
TACTTCACCAACATGTTTGCCACCTGGAGCCCCAGCAAGGCCAGGCTGCACCTGCAGGGCAGGAGCAA
TGCCTGGAGGCCCCAGGTCAACAACCCCAAGGAGTGGCTGCAGGTGGACTTCCAGAAGACCATGAAGG
TGACTGGGGTGACCACCCAGGGGGTGAAGAGCCTGCTGACCAGCATGTATGTGAAGGAGTTCCTGATC
AGCAGCAGCCAGGATGGCCACCAGTGGACCCTGTTCTTCCAGAATGGCAAGGTGAAGGTGTTCCAGGG
CAACCAGGACAGCTTCACCCCTGTGGTGAACAGCCTGGACCCCCCCCTGCTGACCAGATACCTGAGGA
TTCACCCCCAGAGCTGGGTGCACCAGATTGCCCTGAGGATGGAGGTGCTGGGCTGTGAGGCCCAGGAC
CTGTACTGATTAATTAAGAGCATCTTACCGCCATTTATTCCCATATTTGTTCTGTTTTTCTTGATTTG
GGTATACATTTAAATGTTAATAAAACAAAATGGTGGGGCAATCATTTACATTTTTAGGGATATGTAAT
TACTAGTTCAGGTGTATTGCCACAAGACAAACATGTTAAGAAACTTTCCCGTTATTTACGCTCTGTTC
CTGTTAATCAACCTCTGGATTACAAAATTTGTGAAAGATTGACTGATATTCTTAACTATGTTGCTCCT
TTTACGCTGTGTGGATATGCTGCTTTATAGCCTCTGTATCTAGCTATTGCTTCCCGTACGGCTTTCGT
TTTCTCCTCCTTGTATAAATCCTGGTTGCTGTCTCTTTTAGAGGAGTTGTGGCCCGTTGTCCGTCAAC
GTGGCGTGGTGTGCTCTGTGTTTGCTGACGCAACCCCCACTGGCTGGGGCATTGCCACCACCTGTCAA
CTCCTTTCTGGGACTTTCGCTTTCCCCCTCCCGATCGCCACGGCAGAACTCATCGCCGCCTGCCTTGC
CCGCTGCTGGACAGGGGCTAGGTTGCTGGGCACTGATAATTCCGTGGTGTTGTCTGTGCCTTCTAGTT
GCCAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGGGATGCGGTGGGCT
CTATGGCTCTAGAGCATGGCTACGTAGATAAGTAGCATGGCGGGTTAATCATTAACTACACCTGCAGG
AGGAACCCCTAGTGATGGAGTTGGCCACTCCCTCTCTGCGCGCTCGCTCGCTCACTGAGGCCGGGCGA
CCAAAGGTCGCCCGACGCCCGGGCGGCCTCAGTGAGCGAGCGAGCGCGCAGCTGCCTGCAGG

### Example 4: ceDNA with Human SERPINA1 Enhancer Spacer Variants Exhibited at Least Equivalent or Superior Factor VIII In Vivo Expression in Rag2 Mice when Formulated as LNP Compositions

The objective of this study was to determine and compare the effect of LNP-formulated ceDNA on in vivo expression in male Rag2 mice. where The ceDNA comprising a Factor VIII transgene, as regulated by: (i) 3× version of hSerpEnh enhancer (3×_SerpEnh-control; 1 basepair spapcer “1 mer”); (ii) 3× version of hSerpEnh enhancer with 2-mer spacers (3×_hSerpEnh_“2mer” 2 bp spacers_v9) placed between hSerpEnh enhancer element repeats; or (iii) 3× version of hSerpEnh enhancer with 11-mer spacers (3×_hSerpEnh_11mer spacers_FOXA) placed between hSerpEnh element repeats (see Table 5).

TABLE 5
Test material administration

| | | | Dose | Dose | Dosing | Terminal |
|---|---|---|---|---|---|---|
| Group | No. of | | Levels | Volume | Regimen | Time |
| No. | Animals | Test Material | (mg/kg) | (mL/kg) | ROA | Point |
| 1 | 5 | PBS | 0 | 5 | Once on | Day 42 |
| 2 | 5 | 3x_hSerpEnh | 0.5 | | Day 0 by IV | |
| | | (Control) | | | | |
| 3 | 5 | 3x_hSerpEnh | 2.0 | | | |
| | | (Control) | | | | |
| 4 | 5 | 3x_hSerpEnh_2mer_{—} | 0.5 | | | |
| | | spacers_v9 | | | | |
| 5 | 5 | 3x_hSerpEnh_2mer_{—} | 2.0 | | | |
| | | spacers_v9 | | | | |
| 6 | 5 | 3x_hSerpEnh_11mer_{—} | 0.5 | | | |
| | | spacers_FOXA | | | | |
| 7 | 5 | 3x_hSerpEnh_11mer_{—} | 2.0 | | | |
| | | spacers_FOXA | | | | |

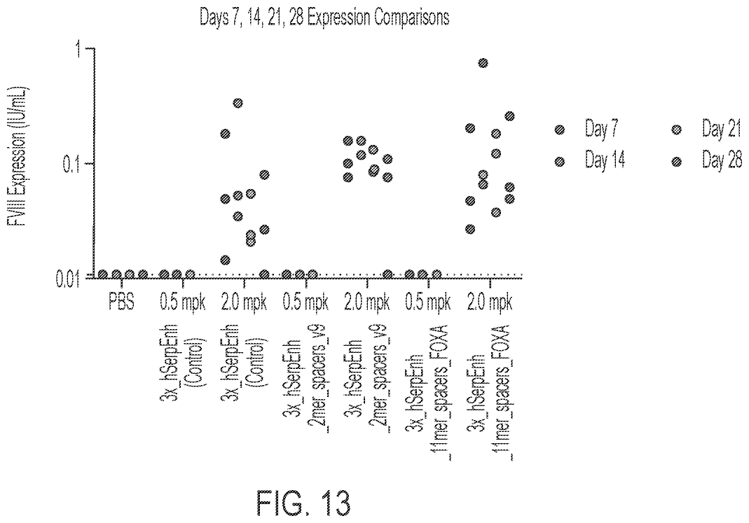

The mice were dosed intravenously once at Day 0 at a low dose of 0.5 mg/kg or a high dose of 2.0 mg/kg (n=5) and the Factor VIII expression was measured at Days 7, 14, 21, and 28. As shown in FIG. 13, the treated mice exhibited dose-dependent response to the administered LNP formulations e comprising various ceDNA 3× hSerpEnh spacer variants listed above in that a high dose of 2.0 mg/kg consistently resulted in higher Factor VIII expression in the mice, as compared to a low dose of 0.5 mg/kg. Additionally, it was observed that the 3× version of hSerpEnh enhancers that have either 2 bp or 11 bp spacer exhibited at least equivalent or higher Factor VIII expression in the mice, as compared to the control 3×_hSerpEnh enhancer having a 1 bp spacer.

### Example 5: ceDNA with Chinese Tree Shrew SERPINA1 Enhancer Variants Exhibited at Least Equivalent or Superior Factor VIII In Vivo Expression in C57BL/6J Mice when Administered Via Hydrodynamic Tail Vein Injection

The objective of this study was to determine and compare the effect of ceDNA on in vivo expression in male C57BL/6J mice. The ceDNA comprised a Factor VIII transgene, as regulated by: (i) 3× version of hSerpEnh enhancer (3×_SerpEnh with 1 bp spacer “1 mer”)—control); (ii) 3× version of Chinese Tree Shrew SerpEnh enhancer (3×_ChineseTreeShrew with 1 bp spacer); (iii) 3× version of hSerpEnh enhancer with HNF4 and FOXA transcription factor consensus sites and secondary structure formation minimization (3×_HNF4_FOXA_v1_SecondaryStruct_min_v2 with 1 bp spacer); or (iv) 3× version of Chinese Tree Shrew SerpEnh enhancer with CpG minimization (3×_ChineseTreeShrew_CpG_min with 1 bp spacer) (see Table 6).

TABLE 6
Test material administration

| | | | Dose | Dose | Administration | Dosage |
|---|---|---|---|---|---|---|
| | No. | | Levels | Volume | Route of | Regimen/ |
| Group | Animals | Test Material | (μg/an) | (ml/kg) | (ROA) | Frequency |
| A | 5 | PBS | N/A | 90-100 | HDIV | Once on |
| B | 5 | 3x_hSerpEnh | 50 ng | ml/kg | | Day 0 |
| | | (Control) | | | | |
| C | 5 | 3x_ChineseTreeShrew | | | | |
| D | 5 | 3x_HNF4_FOXA_v1_{—} | | | | |
| | | SecondaryStruct_min_v2 | | | | |
| E | 5 | 3x_ChineseTreeShrew_{—} | | | | |
| | | CpG_min | | | | |

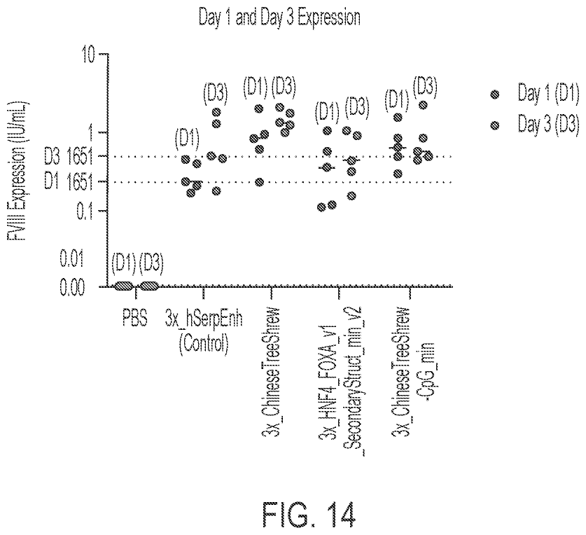

The mice were dosed intravenously via hydrodynamic tail vein injection once at Day 0 at a dose of 50 ng (n=5) and the Factor VIII expression was measured at Days 1 and 3. As shown in FIG. 14, the 3× version of Chinese Tree Shrew SerpEnh enhancers and 3× version of hSerpEnh enhancer with HNF4 and FOXA transcription factor consensus sites and secondary structure formation minimization exhibited at least equivalent or higher Factor VIII expression in the mice, as compared to the control 3×_hSerpEnh enhancer.

### Example 6: ceDNA with Bushbaby SERPINA1 Enhancer Variants Exhibited Superior Factor VIII In Vivo Expression in C57BL/6J Mice when Administered Via Hydrodynamic Tail Vein Injection

The objective of this study was to determine and compare the effect of ceDNA on in vivo expression in male C57BL/6J mice, whereby the ceDNA comprised a Factor VIII transgene, as regulated by: (i) 3× version of hSerpEnh enhancer (3×_SerpEnh—positive control); (ii) 3× version of Bushbaby SerpEnh enhancer with adenosine spacer between every two copies of the enhancer (3×_Bushbaby_Aspacers—Sample 1); or (iii) 3× version of Bushbaby SerpEnh enhancer with adenosine spacer between every two copies of the enhancer (3×_Bushbaby_Aspacers—Sample 2); (see Table 7).

TABLE 7
Test material administration

| | No. | | Dose Levels | |
|---|---|---|---|---|
| Group | Animals | Test Material | (μg/an) | Endpoint |
| A | 5 | PBS | 10 ng on Day 0 | Citrate |
| B | 5 | 3x_hSerpEnh | | plasma on |
| | | (Control) | | Day 3 |
| C | 5 | 3x_Bushbaby_Aspacers | | |
| | | (Sample 1) | | |
| D | 5 | 3x_Bushbaby_Aspacers | | |
| | | (Sample 2) | | |

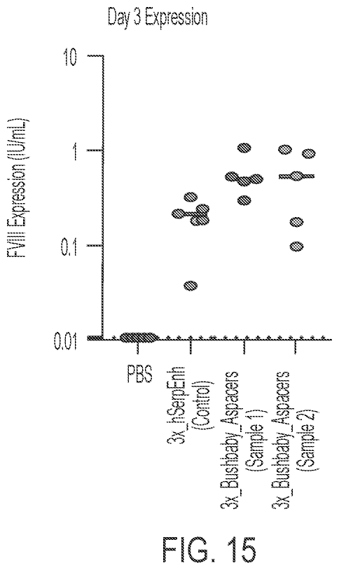

The mice were dosed hydrodynamically via tail vein injection once at Day 0 at a dose of 10 ng (n=5) and the Factor VIII expression was measured at Day 3. As shown in FIG. 15, the 3× version of Bushbaby SerpEnh enhancer with adenosine nucleotide spacers exhibited higher Factor VIII expression in the mice, as compared to the control 3×_hSerpEnh enhancer.

### Example 7: ceDNA with Armadillo or Tibetan Antelope SERPINA1 Enhancer Variants Exhibited at Least Equivalent Factor VIII In Vivo Expression in C57BL/6J Mice when Administered Via Hydrodynamic Tail Vein Injection

The objective of this study was to determine and compare the effect of ceDNA on in vivo expression in male C57BL/6J mice, whereby the ceDNA comprised a Factor VIII transgene, as regulated by: (i) 3× version of hSerpEnh enhancer (3× SerpEnh-control); (ii) 3× version of Tibetan Antelope SerpEnh enhancer (3×50ibetan_antelopeSERPINA1_enhancer); or (iii) 3× version of Armadillo SerpEnh enhancer with CpG minimization (3× Armadillo_CpGminSERPINA1 enhancer) (see Table 8).

TABLE 8
Test material administration

| | | | Dose | Dose | Dosing | Terminal |
|---|---|---|---|---|---|---|
| Group | No. of | | Levels | Volume | Regimen | Time |
| No. | Animals | Test Material | (ng/an) | (mL/kg) | ROA | Point |
| 1 | 4 | PBS | 0 | 90-100 | Once on | Day 3 |
| 2 | 4 | 3x_hSerpEnh | 25 | (set volume) | Day 0 by | |
| 3 | 4 | (Control) | 50 | | HDIV | |
| 4 | 4 | | 100 | | | |
| 5 | 4 | 3x_Tibetan_antelope_{—} | 25 | | | |
| 6 | 4 | SERPINA1_enhancer | 50 | | | |
| 7 | 4 | | 100 | | | |
| 8 | 4 | 3x_Armadillo_CpGmin_{—} | 25 | | | |
| 9 | 4 | SERPINA1_enhancer | 50 | | | |
| 10 | 4 | | 100 | | | |
| 11 | 4 | 3x_ChineseTreeShrew | 25 | | | |
| 12 | 4 | | 50 | | | |
| 13 | 4 | | 100 | | | |
| 14 | 4 | 3x_ChineseTreeShrew_{—} | 25 | | | |
| 15 | 4 | CpGmin | 50 | | | |
| 16 | 4 | | 100 | | | |
| 17 | 4 | 3x_Bushbaby_Aspacers | 25 | | | |
| 18 | 4 | | 50 | | | |
| 19 | 4 | | 100 | | | |

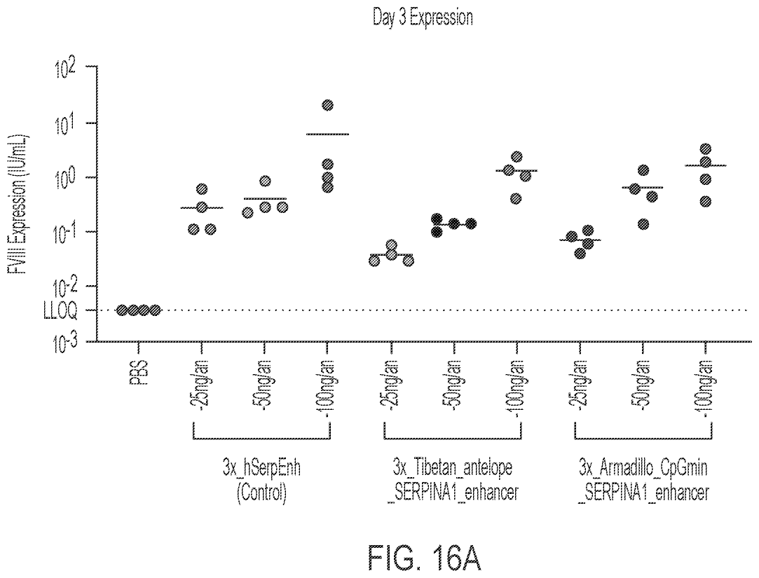

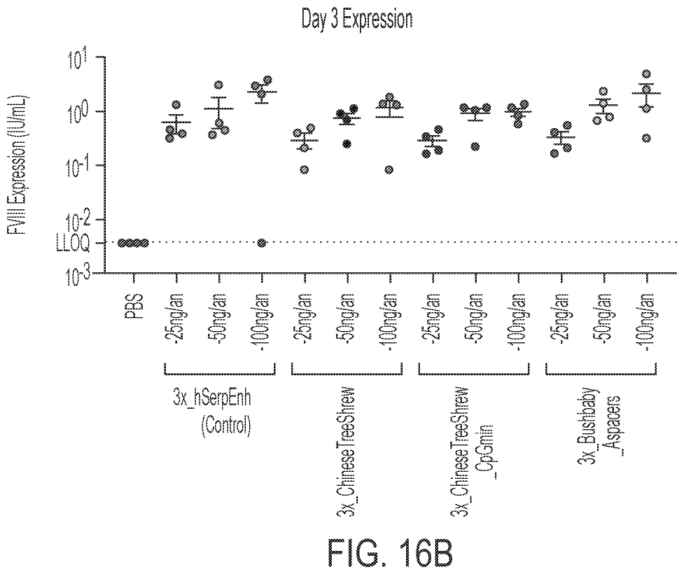

The mice were dosed intravenously via hydrodynamic tail vein injection once at Day 0 at three different dose levels: 25 ng/an, 50 ng/an, 100 ng/an (n=4) and the Factor VIII expression was measured at Day 3. As shown in FIG. 16A and FIG. 16B, the administered ceDNA constructs were dose-responsive in that higher doses of 50 ng/an and 100 ng/an resulted in higher Factor VIII expression in the mice, as compared to a low dose of 25 ng/an. Additionally, it was observed that all tested enhancers exhibited at least equivalent Factor VIII expression in the mice, as compared to the control 3×_hSerpEnh enhancer.

### Example 8: High-Throughput Screening of Human SERPINA1 Enhancer Variants, BushBaby SERPINA1 Enhancer Variants, Chinese Tree Shrew SERPINA1 Enhancer Variants, and Human SERPINA1 Enhancer Variants with HNF4 and FOXA Transcription Factor Consensus Sites

The following enhancer sequence variants were generated to evaluate their ability to drive expression by high-throughput expression screening:
(1) all single nucleotide substitution and adjacent di-nucleotide substitution variants for human SERPINA1 enhancer (e.g., single nucleotide variants and adjacent di-nucleotide variants of SEQ ID NO: 81), Chinese Tree Shrew modified SERPINA1 enhancer (e.g.,. single nucleotide substitution and adjacent di-nucleotide substitution variants for SEQ ID NO: 122), BushBaby SERPINA1 enhancer (e.g., single nucleotide variants and adjacent di-nucleotide variants of SEQ ID NO: 83), and human SERPINA1 enhancer variants with HNF4 and FOXA transcription factor consensus sites HNF4_FOXA enhancer i.e., single nucleotide variants and adjacent di-nucleotide variants of SEQ ID NO: 85);
(2) selected variants of the human SERPINA1 enhancer with four modified nucleotides (i.e., four nucleotide substitution variants of SEQ ID NO: 81); and
(3) 50 random permutations of the human SERPINA1 enhancer used as a negative control (i.e., random permutations of SEQ ID NO: 81). In total, 5,151 unique sequences were screened. To map sequencing reads back to enhancer sequences, all screened sequenced were associated with one or more 10 nucleotide barcodes with a minimum Levenshtein distance of two nucleotides between all barcodes. Original enhancer sequences were each associated with 200 barcodes and all variants were associated with one barcode. Enhancer sequences and barcodes were generated with custom MATLAB scripts.

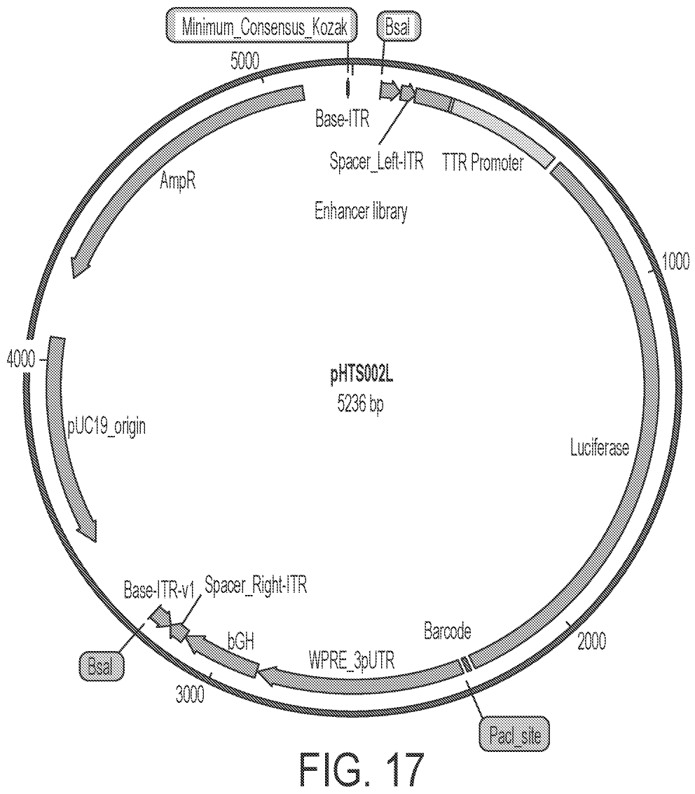

An oligo pool of the enhancers was ordered from Twist Biosciences and the plasmid library, pHTS002L (FIG. 17), with luciferase as the reporter gene, was constructed. HepG2 cells (ATCC, VA) were cultured in DMEM (Dulbecco's Modified Eagle Medium) medium (ThermoFisher, MA) with 10% fetal bovine serum (ThermoFisher, MA) at 37° C. The day before transfection, cells were harvested with 0.25% trypsin (ThermoFisher, MA) and seeded at the density of 600,000 cells per well on 6-well collagen-coated plates (VWR, PA). 2 ug plasmid were transfected per well with TransfeX (ATCC, VA) according to the manufacture's manual. The cells were harvested 24 hours after transfection and total RNA were extracted with the RNAeasy plus kit (Qiagen, Germany). 1 ug RNA or 10 ng plasmid DNA were used for amplicon production using the primers in Table 9 with SuperScript™ IV One-Step RT-PCR System (ThermoFisher, MA) according to the manufacture's manual. Amplicons contained the barcode associated with each enhancer. The concentrations of the 6 indexed amplicons were measured with Qubit (ThermoFisher, MA). The amplicons were sequenced with Illumina Miseq (75 bp×2) at MIT BioMicro Center.

TABLE 9
Primers used for amplicon production

| | | |
|---|---|---|
| Forward | SEQ ID | AATGATACGGCGACCACCGAGATCTACACTCTTTCCCTACACGACGCTCT |
| | NO: 148 | TCCGATCTGGAGGGAAGATTGCTGTGTGATAG |
| Reverse 1 | SEQ ID | CAAGCAGAAGACGGCATACGAGATGTGACTGTGACTGGAGTTCAGACGTG |
| | NO: 149 | TGCTCTTCCGATCT CAG GAA CAG AGC GTA AAT AAC GGG |
| Reverse 2 | SEQ ID | CAAGCAGAAGACGGCATACGAGATCTGCAAGTGACTGGAGTTCAGACGTG |
| | NO: 150 | TGCTCTTCCGATCT CAG GAA CAG AGC GTA AAT AAC GGG |
| Reverse 3 | SEQ ID | CAAGCAGAAGACGGCATACGAGATACCATGGTGACTGGAGTTCAGACGTG |
| | NO: 151 | TGCTCTTCCGATCT CAG GAA CAG AGC GTA AAT AAC GGG |
| Reverse 4 | SEQ ID | CAAGCAGAAGACGGCATACGAGATGAACGTGTGACTGGAGTTCAGACGTG |
| | NO: 152 | TGCTCTTCCGATCT CAG GAA CAG AGC GTA AAT AAC GGG |
| Reverse 5 | SEQ ID | CAAGCAGAAGACGGCATACGAGATACTAGTGTGACTGGAGTTCAGACGTG |
| | NO: 153 | TGCTCTTCCGATCT CAG GAA CAG AGC GTA AAT AAC GGG |
| Reverse 6 | SEQ ID | CAAGCAGAAGACGGCATACGAGATCGTTACGTGACTGGAGTTCAGACGTG |
| | NO: 154 | TGCTCTTCCGATCT CAG GAA CAG AGC GTA AAT AAC GGG |

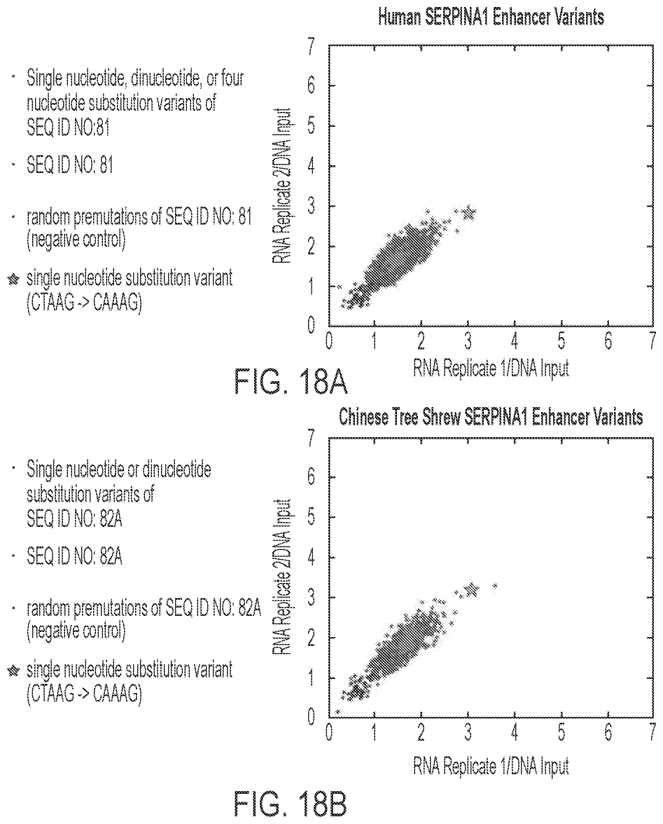

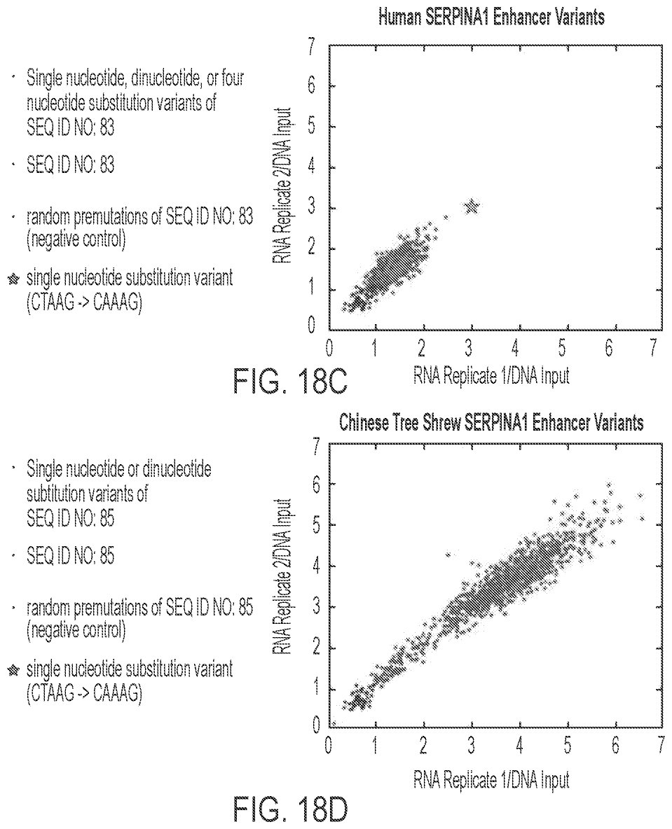

Reads were filtered out that (1) did not contain the expected sequence for the 10 nucleotides up- and downstream of the barcode and (2) contained quality scores less than 20 in the barcode (FASTX-Toolkit). Barcode counts for each RNA sample were normalized to the corresponding barcode counts for an input DNA sample and mapped back to their associated enhancer sequences (custom MATLAB script). Comparisons for two biological replicates are shown in FIGS. 18A-18D.

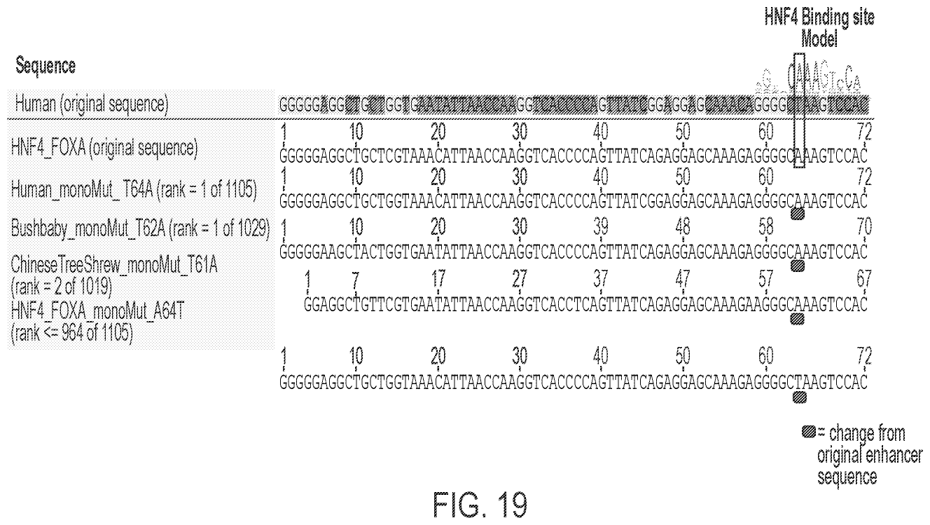

The enhancer variants that exhibited higher expression levels than the expression levels of their respective original sequences (i.e., SEQ ID NOs: 81, 122, 83, or 85) are listed in Table 10 (human SERPINA1 enhancer variants); Table 11 (Chinese Tree Shrew SERPINA1 enhancer variants), Table 12 (BushBaby SERPINA 1 enhancer variants, and Table 13 (human SERPINA1 enhancer variants with HNF4 and FOXA transcription factor consensus sites). Of note, single nucleotide substitution variants of the human SERPINA1 enhancer, single nucleotide substitution variants of the Chinese Tree Shrew SERPINA1 enhancer, and single nucleotide substitution variants of the Bushbaby SERPINA1 enhancer that each carry a CTAAG -> CAAAG mutation were consistently among the highest expression variants among their respective variant populations (see FIGS. 18A-18C). As indicated in FIG. 19 that shows the alignment of multiple SERPINA1 enhancer sequences, the CAAAG sequence is located in the HNF4 transcription factor consensus site in SEQ ID NO: 85, the sequence of the human SERPINA1 enhancer variants with HNF4 and FOXA transcription factor consensus sites HNF4_FOXA enhancer. When the CAAAG sequence is modified to CTAAG, the expression levels of the reciprocal variant were compromised (see FIG. 18D).

TABLE 10
Single, adjacent di-, or four nucleotide substitution variants of human SERPINA1 enhancer with higher luciferase
expression than original sequence SEQ ID NO: 81

| SEQ ID | | |
|---|---|---|
| NO: | Human SERPINA1 enhancer variant | Sequence |
| 155 | 202_Human_monoMut_G1C_n1 | CGGGGAGGCTGCTGGTGAATATTAACCAAGGTCACCCCAGITATCGGAGGAGCAAACAGGGGCTAAGTCCA |
| | | C |
| 156 | 204_Human_monoMut_G2A_n1 | GAGGGAGGCTGCTGGTGAATATTAACCAAGGTCACCCCAGTTATCGGAGGAGCAAACAGGGGCTAAGTCCA |
| | | C |
| 157 | 206_Human_monoMut_G2T_n1 | GTGGGAGGCTGCTGGTGAATATTAACCAAGGTCACCCCAGTTATCGGAGGAGCAAACAGGGGCTAAGTCCA |
| | | C |
| 158 | 210_Human_monoMut_G4A_n1 | GGGAGAGGCTGCTGGTGAATATTAACCAAGGTCACCCCAGTTATCGGAGGAGCAAACAGGGGCTAAGTCCA |
| | | C |
| 159 | 214_Human_monoMut_G5C_n1 | GGGGCAGGCTGCTGGTGAATATTAACCAAGGTCACCCCAGTTATCGGAGGAGCAAACAGGGGCTAAGTCCA |
| | | C |
| 160 | 216_Human_monoMut_A6C_n1 | GGGGGCGGCTGCTGGTGAATATTAACCAAGGTCACCCCAGTTATCGGAGGAGCAAACAGGGGCTAAGTCCA |
| | | C |
| 161 | 220_Human_monoMut_G7C_n1 | GGGGGACGCTGCTGGTGAATATTAACCAAGGTCACCCCAGTTATCGGAGGAGCAAACAGGGGCTAAGTCCA |
| | | C |
| 162 | 225_Human_monoMut_C9A_n1 | GGGGGAGGATGCTGGTGAATATTAACCAAGGTCACCCCAGTTATCGGAGGAGCAAACAGGGGCTAAGTCCA |
| | | C |
| 163 | 226_Human_monoMut_C9G_n1 | GGGGGAGGGTGCTGGTGAATATTAACCAAGGTCACCCCAGTTATCGGAGGAGCAAACAGGGGCTAAGTCCA |
| | | C |
| 164 | 229_Human_monoMut_T10C_n1 | GGGGGAGGCCGCTGGTGAATATTAACCAAGGTCACCCCAGTTATCGGAGGAGCAAACAGGGGCTAAGTCCA |
| | | C |
| 165 | 230_Human_monoMut_T10G_n1 | GGGGGAGGCGGCTGGTGAATATTAACCAAGGTCACCCCAGTTATCGGAGGAGCAAACAGGGGCTAAGTCCA |
| | | C |
| 166 | 235_Human_monoMut_C12G_n1 | GGGGGAGGCTGGTGGTGAATATTAACCAAGGTCACCCCAGTTATCGGAGGAGCAAACAGGGGCTAAGTCCA |
| | | C |
| 167 | 236_Human_monoMut_C12T_n1 | GGGGGAGGCTGTTGGTGAATATTAACCAAGGTCACCCCAGTTATCGGAGGAGCAAACAGGGGCTAAGTCCA |
| | | C |
| 168 | 245_Human_monoMut_G15T_n1 | GGGGGAGGCTGCTGTTGAATATTAACCAAGGTCACCCCAGTTATCGGAGGAGCAAACAGGGGCTAAGTCCA |
| | | C |
| 169 | 247_Human_monoMut_T16C_n1 | GGGGGAGGCTGCTGGCGAATATTAACCAAGGTCACCCCAGTTATCGGAGGAGCAAACAGGGGCTAAGTCCA |
| | | C |
| 170 | 250_Human_monoMut_G17C_n1 | GGGGGAGGCTGCTGGTCAATATTAACCAAGGTCACCCCAGITATCGGAGGAGCAAACAGGGGCTAAGTCCA |
| | | C |
| 171 | 251_Human_monoMut_G17T_n1 | GGGGGAGGCTGCTGGTTAATATTAACCAAGGTCACCCCAGTTATCGGAGGAGCAAACAGGGGCTAAGTCCA |
| | | C |
| 172 | 260_Human_monoMut_T20G_n1 | GGGGGAGGCTGCTGGTGAAGATTAACCAAGGTCACCCCAGTTATCGGAGGAGCAAACAGGGGCTAAGTCCA |
| | | C |
| 173 | 261_Human_monoMut_A21C_n1 | GGGGGAGGCTGCTGGTGAATCTTAACCAAGGTCACCCCAGTTATCGGAGGAGCAAACAGGGGCTAAGTCCA |
| | | C |
| 174 | 263_Human_monoMut_A21T_n1 | GGGGGAGGCTGCTGGTGAATTTTAACCAAGGTCACCCCAGTTATCGGAGGAGCAAACAGGGGCTAAGTCCA |
| | | C |
| 175 | 264_Human_monoMut_T22A_n1 | GGGGGAGGCTGCTGGTGAATAATAACCAAGGTCACCCCAGTTATCGGAGGAGCAAACAGGGGCTAAGTCCA |
| | | C |
| 176 | 269_Human_monoMut_T23G_n1 | GGGGGAGGCTGCTGGTGAATATGAACCAAGGTCACCCCAGTTATCGGAGGAGCAAACAGGGGCTAAGTCCA |
| | | C |
| 177 | 270_Human_monoMut_A24C_n1 | GGGGGAGGCTGCTGGTGAATATTCACCAAGGTCACCCCAGTTATCGGAGGAGCAAACAGGGGCTAAGTCCA |
| | | C |
| 178 | 274_Human_monoMut_A25G_n1 | GGGGGAGGCTGCTGGTGAATATTAGCCAAGGTCACCCCAGTTATCGGAGGAGCAAACAGGGGCTAAGTCCA |
| | | C |
| 179 | 275_Human_monoMut_A25T_n1 | GGGGGAGGCTGCTGGTGAATATTATCCAAGGTCACCCCAGTTATCGGAGGAGCAAACAGGGGCTAAGTCCA |
| | | C |
| 180 | 278_Human_monoMut_C26T_n1 | GGGGGAGGCTGCTGGTGAATATTAATCAAGGTCACCCCAGTTATCGGAGGAGCAAACAGGGGCTAAGTCCA |
| | | C |
| 181 | 282_Human_monoMut_A28C_n1 | GGGGGAGGCTGCTGGTGAATATTAACCCAGGTCACCCCAGTTATCGGAGGAGCAAACAGGGGCTAAGTCCA |
| | | C |
| 182 | 283_Human_monoMut_A28G_n1 | GGGGGAGGCTGCTGGTGAATATTAACCGAGGTCACCCCAGTTATCGGAGGAGCAAACAGGGGCTAAGTCCA |
| | | C |
| 183 | 284_Human_monoMut_A28T_n1 | GGGGGAGGCTGCTGGTGAATATTAACCTAGGTCACCCCAGTTATCGGAGGAGCAAACAGGGGCTAAGTCCA |
| | | C |
| 184 | 295_Human_monoMut_T32C_n1 | GGGGGAGGCTGCTGGTGAATATTAACCAAGGCCACCCCAGTTATCGGAGGAGCAAACAGGGGCTAAGTCCA |
| | | C |
| 185 | 301_Human_monoMut_A34G_n1 | GGGGGAGGCTGCTGGTGAATATTAACCAAGGTCGCCCCAGTTATCGGAGGAGCAAACAGGGGCTAAGTCCA |
| | | C |
| 186 | 303_Human_monoMut_C35A_n1 | GGGGGAGGCTGCTGGTGAATATTAACCAAGGTCAACCCAGTTATCGGAGGAGCAAACAGGGGCTAAGTCCA |
| | | C |
| 187 | 304_Human_monoMut_C35G_n1 | GGGGGAGGCTGCTGGTGAATATTAACCAAGGTCAGCCCAGTTATCGGAGGAGCAAACAGGGGCTAAGTCCA |
| | | C |
| 188 | 305_Human_monoMut_C35T_n1 | GGGGGAGGCTGCTGGTGAATATTAACCAAGGTCATCCCAGTTATCGGAGGAGCAAACAGGGGCTAAGTCCA |
| | | C |
| 189 | 308_Human_monoMut_C36T_n1 | GGGGGAGGCTGCTGGTGAATATTAACCAAGGTCACTCCAGTTATCGGAGGAGCAAACAGGGGCTAAGTCCA |
| | | C |
| 190 | 314_Human_monoMut_C38T_n1 | GGGGGAGGCTGCTGGTGAATATTAACCAAGGTCACCCTAGTTATCGGAGGAGCAAACAGGGGCTAAGTCCA |
| | | C |
| 191 | 315_Human_monoMut_A39C_n1 | GGGGGAGGCTGCTGGTGAATATTAACCAAGGTCACCCCCGTTATCGGAGGAGCAAACAGGGGCTAAGTCCA |
| | | C |
| 192 | 316_Human_monoMut_A39G_n1 | GGGGGAGGCTGCTGGTGAATATTAACCAAGGTCACCCCGGTTATCGGAGGAGCAAACAGGGGCTAAGTCCA |
| | | C |
| 193 | 317_Human_monoMut_A39T_n1 | GGGGGAGGCTGCTGGTGAATATTAACCAAGGTCACCCCTGTTATCGGAGGAGCAAACAGGGGCTAAGTCCA |
| | | C |
| 194 | 325_Human_monoMut_T42C_n1 | GGGGGAGGCTGCTGGTGAATATTAACCAAGGTCACCCCAGTCATCGGAGGAGCAAACAGGGGCTAAGTCCA |
| | | C |
| 195 | 328_Human_monoMut_A43G_n1 | GGGGGAGGCTGCTGGTGAATATTAACCAAGGTCACCCCAGTIGTCGGAGGAGCAAACAGGGGCTAAGTCCA |
| | | C |
| 196 | 331_Human_monoMut_T44C_n1 | GGGGGAGGCTGCTGGTGAATATTAACCAAGGTCACCCCAGTTACCGGAGGAGCAAACAGGGGCTAAGTCCA |
| | | C |
| 197 | 332_Human_monoMut_T44G_n1 | GGGGGAGGCTGCTGGTGAATATTAACCAAGGTCACCCCAGTTAGCGGAGGAGCAAACAGGGGCTAAGTCCA |
| | | C |
| 198 | 333_Human_monoMut_C45A_n1 | GGGGGAGGCTGCTGGTGAATATTAACCAAGGTCACCCCAGTTATAGGAGGAGCAAACAGGGGCTAAGTCCA |
| | | C |
| 199 | 334_Human_monoMut_C45G_n1 | GGGGGAGGCTGCTGGTGAATATTAACCAAGGTCACCCCAGTTATGGGAGGAGCAAACAGGGGCTAAGTCCA |
| | | C |
| 200 | 337_Human_monoMut_G46C_n1 | GGGGGAGGCTGCTGGTGAATATTAACCAAGGTCACCCCAGTTATCCGAGGAGCAAACAGGGGCTAAGTCCA |
| | | C |
| 201 | 344_Human_monoMut_A48T_n1 | GGGGGAGGCTGCTGGTGAATATTAACCAAGGTCACCCCAGTTATCGGTGGAGCAAACAGGGGCTAAGTCCA |
| | | C |
| 202 | 345_Human_monoMut_G49A_n1 | GGGGGAGGCTGCTGGTGAATATTAACCAAGGTCACCCCAGTTATCGGAAGAGCAAACAGGGGCTAAGTCCA |
| | | C |
| 203 | 346_Human_monoMut_G49C_n1 | GGGGGAGGCTGCTGGTGAATATTAACCAAGGTCACCCCAGTTATCGGACGAGCAAACAGGGGCTAAGTCCA |
| | | C |
| 204 | 347_Human_monoMut_G49T_n1 | GGGGGAGGCTGCTGGTGAATATTAACCAAGGTCACCCCAGTTATCGGATGAGCAAACAGGGGCTAAGTCCA |
| | | C |
| 205 | 351_Human_monoMut_A51C_n1 | GGGGGAGGCTGCTGGTGAATATTAACCAAGGTCACCCCAGTTATCGGAGGCGCAAACAGGGGCTAAGTCCA |
| | | C |
| 206 | 353_Human_monoMut_A51T_n1 | GGGGGAGGCTGCTGGTGAATATTAACCAAGGTCACCCCAGTTATCGGAGGTGCAAACAGGGGCTAAGTCCA |
| | | C |
| 207 | 354_Human_monoMut_G52A_n1 | GGGGGAGGCTGCTGGTGAATATTAACCAAGGTCACCCCAGTTATCGGAGGAACAAACAGGGGCTAAGTCCA |
| | | C |
| 208 | 356_Human_monoMut_G52T_n1 | GGGGGAGGCTGCTGGTGAATATTAACCAAGGTCACCCCAGTTATCGGAGGATCAAACAGGGGCTAAGTCCA |
| | | C |
| 209 | 371_Human_monoMut_C57T_n1 | GGGGGAGGCTGCTGGTGAATATTAACCAAGGTCACCCCAGTTATCGGAGGAGCAAATAGGGGCTAAGTCCA |
| | | C |
| 218 | 375_Human_monoMut_G59A_n1 | GGGGGAGGCTGCTGGTGAATATTAACCAAGGTCACCCCAGTTATCGGAGGAGCAAACAAGGGCTAAGTCCA |
| | | C |
| 219 | 380_Human_monoMut_G60T_n1 | GGGGGAGGCTGCTGGTGAATATTAACCAAGGTCACCCCAGTTATCGGAGGAGCAAACAGTGGCTAAGTCCA |
| | | C |
| 220 | 381_Human_monoMut_G61A_n1 | GGGGGAGGCTGCTGGTGAATATTAACCAAGGTCACCCCAGTTATCGGAGGAGCAAACAGGAGCTAAGTCCA |
| | | C |
| 221 | 383_Human_monoMut_G61T_n1 | GGGGGAGGCTGCTGGTGAATATTAACCAAGGTCACCCCAGTTATCGGAGGAGCAAACAGGTGCTAAGTCCA |
| | | C |
| 222 | 386_Human_monoMut_G62T_n1 | GGGGGAGGCTGCTGGTGAATATTAACCAAGGTCACCCCAGTTATCGGAGGAGCAAACAGGGTCTAAGTCCA |
| | | C |
| 223 | 390_Human_monoMut_T64A_n1 | GGGGGAGGCTGCTGGTGAATATTAACCAAGGTCACCCCAGTTATCGGAGGAGCAAACAGGGGCAAAGTCCA |
| | | C |
| 224 | 392_Human_monoMut_T64G_n1 | GGGGGAGGCTGCTGGTGAATATTAACCAAGGTCACCCCAGTTATCGGAGGAGCAAACAGGGGCGAAGTCCA |
| | | C |
| 225 | 394_Human_monoMut_A65G_n1 | GGGGGAGGCTGCTGGTGAATATTAACCAAGGTCACCCCAGTTATCGGAGGAGCAAACAGGGGCTGAGTCCA |
| | | C |
| 226 | 405_Human_monoMut_C69A_n1 | GGGGGAGGCTGCTGGTGAATATTAACCAAGGTCACCCCAGTTATCGGAGGAGCAAACAGGGGCTAAGTACA |
| | | C |
| 227 | 414_Human_monoMut_C72A_n1 | GGGGGAGGCTGCTGGTGAATATTAACCAAGGTCACCCCAGTTATCGGAGGAGCAAACAGGGGCTAAGTCCA |
| | | A |
| 228 | 415_Human_monoMut_C72G_n1 | GGGGGAGGCTGCTGGTGAATATTAACCAAGGTCACCCCAGTTATCGGAGGAGCAAACAGGGGCTAAGTCCA |
| | | G |
| 229 | 421_Human_diMut_GG1CC_n1 | CCGGGAGGCTGCTGGTGAATATTAACCAAGGTCACCCCAGTTATCGGAGGAGCAAACAGGGGCTAAGTCCA |
| | | C |
| 230 | 427_Human_diMut_GG2AC_n1 | GACGGAGGCTGCTGGTGAATATTAACCAAGGTCACCCCAGTTATCGGAGGAGCAAACAGGGGCTAAGTCCA |
| | | C |
| 231 | 429_Human_diMut_GG2CA_n1 | GCAGGAGGCTGCTGGTGAATATTAACCAAGGTCACCCCAGTTATCGGAGGAGCAAACAGGGGCTAAGTCCA |
| | | C |
| 232 | 430_Human_diMut_GG2CC_n1 | GCCGGAGGCTGCTGGTGAATATTAACCAAGGTCACCCCAGTTATCGGAGGAGCAAACAGGGGCTAAGTCCA |
| | | C |
| 233 | 439_Human_diMut_GG3CC_n1 | GGCCGAGGCTGCTGGTGAATATTAACCAAGGTCACCCCAGTTATCGGAGGAGCAAACAGGGGCTAAGTCCA |
| | | C |
| 234 | 440_Human_diMut_GG3CT_n1 | GGCTGAGGCTGCTGGTGAATATTAACCAAGGTCACCCCAGTTATCGGAGGAGCAAACAGGGGCTAAGTCCA |
| | | C |
| 235 | 443_Human_diMut_GG3TT_n1 | GGTTGAGGCTGCTGGTGAATATTAACCAAGGTCACCCCAGTTATCGGAGGAGCAAACAGGGGCTAAGTCCA |
| | | C |
| 236 | 444_Human_diMut_GG4AA_n1 | GGGAAAGGCTGCTGGTGAATATTAACCAAGGTCACCCCAGTTATCGGAGGAGCAAACAGGGGCTAAGTCCA |
| | | C |
| 237 | 447_Human_diMut_GG4CA_n1 | GGGCAAGGCTGCTGGTGAATATTAACCAAGGTCACCCCAGTTATCGGAGGAGCAAACAGGGGCTAAGTCCA |
| | | C |
| 238 | 448_Human_diMut_GG4CC_n1 | GGGCCAGGCTGCTGGTGAATATTAACCAAGGTCACCCCAGTTATCGGAGGAGCAAACAGGGGCTAAGTCCA |
| | | C |
| 239 | 452_Human_diMut_GG4TT_n1 | GGGTTAGGCTGCTGGTGAATATTAACCAAGGTCACCCCAGTTATCGGAGGAGCAAACAGGGGCTAAGTCCA |
| | | C |
| 240 | 453_Human_diMut_GA5AC_n1 | GGGGACGGCTGCTGGTGAATATTAACCAAGGTCACCCCAGTTATCGGAGGAGCAAACAGGGGCTAAGTCCA |
| | | C |
| 241 | 456_Human_diMut_GA5CC_n1 | GGGGCCGGCTGCTGGTGAATATTAACCAAGGTCACCCCAGTTATCGGAGGAGCAAACAGGGGCTAAGTCCA |
| | | C |
| 242 | 461_Human_diMut_GA5TT_n1 | GGGGTTGGCTGCTGGTGAATATTAACCAAGGTCACCCCAGTTATCGGAGGAGCAAACAGGGGCTAAGTCCA |
| | | C |
| 243 | 462_Human_diMut_AG6CA_n1 | GGGGGCAGCTGCTGGTGAATATTAACCAAGGTCACCCCAGTTATCGGAGGAGCAAACAGGGGCTAAGTCCA |
| | | C |
| 244 | 464_Human_diMut_AG6CT_n1 | GGGGGCTGCTGCTGGTGAATATTAACCAAGGTCACCCCAGTTATCGGAGGAGCAAACAGGGGCTAAGTCCA |
| | | C |
| 245 | 465_Human_diMut_AG6GA_n1 | GGGGGGAGCTGCTGGTGAATATTAACCAAGGTCACCCCAGTTATCGGAGGAGCAAACAGGGGCTAAGTCCA |
| | | C |
| 246 | 468_Human_diMut_AG6TA_n1 | GGGGGTAGCTGCTGGTGAATATTAACCAAGGTCACCCCAGTTATCGGAGGAGCAAACAGGGGCTAAGTCCA |
| | | C |
| 247 | 469_Human_diMut_AG6TC_n1 | GGGGGTCGCTGCTGGTGAATATTAACCAAGGTCACCCCAGTTATCGGAGGAGCAAACAGGGGCTAAGTCCA |
| | | C |
| 248 | 470_Human_diMut_AG6TT_n1 | GGGGGTTGCTGCTGGTGAATATTAACCAAGGTCACCCCAGTTATCGGAGGAGCAAACAGGGGCTAAGTCCA |
| | | C |
| 249 | 471_Human_diMut_GG7AA_n1 | GGGGGAAACTGCTGGTGAATATTAACCAAGGTCACCCCAGTTATCGGAGGAGCAAACAGGGGCTAAGTCCA |
| | | C |
| 250 | 472_Human_diMut_GG7AC_n1 | GGGGGAACCTGCTGGTGAATATTAACCAAGGTCACCCCAGTTATCGGAGGAGCAAACAGGGGCTAAGTCCA |
| | | C |
| 251 | 473_Human_diMut_GG7AT_n1 | GGGGGAATCTGCTGGTGAATATTAACCAAGGTCACCCCAGTTATCGGAGGAGCAAACAGGGGCTAAGTCCA |
| | | C |
| 252 | 475_Human_diMut_GG7CC_n1 | GGGGGACCCTGCTGGTGAATATTAACCAAGGTCACCCCAGTTATCGGAGGAGCAAACAGGGGCTAAGTCCA |
| | | C |
| 253 | 478_Human_diMut_GG7TC_n1 | GGGGGATCCTGCTGGTGAATATTAACCAAGGTCACCCCAGTTATCGGAGGAGCAAACAGGGGCTAAGTCCA |
| | | C |
| 254 | 482_Human_diMut_GC8AT_n1 | GGGGGAGATTGCTGGTGAATATTAACCAAGGTCACCCCAGTTATCGGAGGAGCAAACAGGGGCTAAGTCCA |
| | | C |
| 255 | 483_Human_diMut_GC8CA_n1 | GGGGGAGCATGCTGGTGAATATTAACCAAGGTCACCCCAGTTATCGGAGGAGCAAACAGGGGCTAAGTCCA |
| | | C |
| 256 | 484_Human_diMut_GC8CG_n1 | GGGGGAGCGTGCTGGTGAATATTAACCAAGGTCACCCCAGTTATCGGAGGAGCAAACAGGGGCTAAGTCCA |
| | | C |
| 257 | 488_Human_diMut_GC8TT_n1 | GGGGGAGTTTGCTGGTGAATATTAACCAAGGTCACCCCAGTTATCGGAGGAGCAAACAGGGGCTAAGTCCA |
| | | C |
| 258 | 490_Human_diMut_CT9AC_n1 | GGGGGAGGACGCTGGTGAATATTAACCAAGGTCACCCCAGTTATCGGAGGAGCAAACAGGGGCTAAGTCCA |
| | | C |
| 259 | 491_Human_diMut_CT9AG_n1 | GGGGGAGGAGGCTGGTGAATATTAACCAAGGTCACCCCAGTTATCGGAGGAGCAAACAGGGGCTAAGTCCA |
| | | C |
| 260 | 492_Human_diMut_CT9GA_n1 | GGGGGAGGGAGCTGGTGAATATTAACCAAGGTCACCCCAGTTATCGGAGGAGCAAACAGGGGCTAAGTCCA |
| | | C |
| 261 | 493_Human_diMut_CT9GC_n1 | GGGGGAGGGCGCTGGTGAATATTAACCAAGGTCACCCCAGTTATCGGAGGAGCAAACAGGGGCTAAGTCCA |
| | | C |
| 262 | 494_Human_diMut_CT9GG_n1 | GGGGGAGGGGGCTGGTGAATATTAACCAAGGTCACCCCAGTTATCGGAGGAGCAAACAGGGGCTAAGTCCA |
| | | C |
| 263 | 495_Human_diMut_CT9TA_n1 | GGGGGAGGTAGCTGGTGAATATTAACCAAGGTCACCCCAGTTATCGGAGGAGCAAACAGGGGCTAAGTCCA |
| | | C |
| 264 | 496_Human_diMut_CT9TC_n1 | GGGGGAGGTCGCTGGTGAATATTAACCAAGGTCACCCCAGTTATCGGAGGAGCAAACAGGGGCTAAGTCCA |
| | | C |
| 265 | 502_Human_diMut_TG10CC_n1 | GGGGGAGGCCCCTGGTGAATATTAACCAAGGTCACCCCAGTTATCGGAGGAGCAAACAGGGGCTAAGTCCA |
| | | C |
| 266 | 503_Human_diMut_TG10CT_n1 | GGGGGAGGCCTCTGGTGAATATTAACCAAGGTCACCCCAGTTATCGGAGGAGCAAACAGGGGCTAAGTCCA |
| | | C |
| 267 | 504_Human_diMut_TG10GA_n1 | GGGGGAGGCGACTGGTGAATATTAACCAAGGTCACCCCAGTTATCGGAGGAGCAAACAGGGGCTAAGTCCA |
| | | C |
| 268 | 506_Human_diMut_TG10GT_n1 | GGGGGAGGCGTCTGGTGAATATTAACCAAGGTCACCCCAGTTATCGGAGGAGCAAACAGGGGCTAAGTCCA |
| | | C |
| 269 | 507_Human_diMut_GC11AA_n1 | GGGGGAGGCTAATGGTGAATATTAACCAAGGTCACCCCAGTTATCGGAGGAGCAAACAGGGGCTAAGTCCA |
| | | C |
| 270 | 510_Human_diMut_GC11CA_n1 | GGGGGAGGCTCATGGTGAATATTAACCAAGGTCACCCCAGTTATCGGAGGAGCAAACAGGGGCTAAGTCCA |
| | | C |
| 271 | 511_Human_diMut_GC11CG_n1 | GGGGGAGGCTCGTGGTGAATATTAACCAAGGTCACCCCAGTTATCGGAGGAGCAAACAGGGGCTAAGTCCA |
| | | C |
| 272 | 512_Human_diMut_GC11CT_n1 | GGGGGAGGCTCTTGGTGAATATTAACCAAGGTCACCCCAGTTATCGGAGGAGCAAACAGGGGCTAAGTCCA |
| | | C |
| 273 | 516_Human_diMut_CT12AA_n1 | GGGGGAGGCTGAAGGTGAATATTAACCAAGGTCACCCCAGTTATCGGAGGAGCAAACAGGGGCTAAGTCCA |
| | | C |
| 274 | 517_Human_diMut_CT12AC_n1 | GGGGGAGGCTGACGGTGAATATTAACCAAGGTCACCCCAGTTATCGGAGGAGCAAACAGGGGCTAAGTCCA |
| | | C |
| 275 | 521_Human_diMut_CT12GG_n1 | GGGGGAGGCTGGGGGTGAATATTAACCAAGGTCACCCCAGTTATCGGAGGAGCAAACAGGGGCTAAGTCCA |
| | | C |
| 276 | 522_Human_diMut_CT12TA_n1 | GGGGGAGGCTGTAGGTGAATATTAACCAAGGTCACCCCAGTTATCGGAGGAGCAAACAGGGGCTAAGTCCA |
| | | C |
| 277 | 523_Human_diMut_CT12TC_n1 | GGGGGAGGCTGTCGGTGAATATTAACCAAGGTCACCCCAGTTATCGGAGGAGCAAACAGGGGCTAAGTCCA |
| | | C |
| 278 | 524_Human_diMut_CT12TG_n1 | GGGGGAGGCTGTGGGTGAATATTAACCAAGGTCACCCCAGTTATCGGAGGAGCAAACAGGGGCTAAGTCCA |
| | | C |
| 279 | 528_Human_diMut_TG13CA_n1 | GGGGGAGGCTGCCAGTGAATATTAACCAAGGTCACCCCAGTTATCGGAGGAGCAAACAGGGGCTAAGTCCA |
| | | C |
| 280 | 544_Human_diMut_GT15AC_n1 | GGGGGAGGCTGCTGACGAATATTAACCAAGGTCACCCCAGTTATCGGAGGAGCAAACAGGGGCTAAGTCCA |
| | | C |
| 281 | 549_Human_diMut_GT15TA_n1 | GGGGGAGGCTGCTGTAGAATATTAACCAAGGTCACCCCAGTTATCGGAGGAGCAAACAGGGGCTAAGTCCA |
| | | C |
| 282 | 551_Human_diMut_GT15TG_n1 | GGGGGAGGCTGCTGTGGAATATTAACCAAGGTCACCCCAGTTATCGGAGGAGCAAACAGGGGCTAAGTCCA |
| | | C |
| 283 | 552_Human_diMut_TG16AA_n1 | GGGGGAGGCTGCTGGAAAATATTAACCAAGGTCACCCCAGTTATCGGAGGAGCAAACAGGGGCTAAGTCCA |
| | | C |
| 284 | 553_Human_diMut_TG16AC_n1 | GGGGGAGGCTGCTGGACAATATTAACCAAGGTCACCCCAGTTATCGGAGGAGCAAACAGGGGCTAAGTCCA |
| | | C |
| 285 | 554_Human_diMut_TG16AT_n1 | GGGGGAGGCTGCTGGATAATATTAACCAAGGTCACCCCAGTTATCGGAGGAGCAAACAGGGGCTAAGTCCA |
| | | C |
| 286 | 555_Human_diMut_TG16CA_n1 | GGGGGAGGCTGCTGGCAAATATTAACCAAGGTCACCCCAGTTATCGGAGGAGCAAACAGGGGCTAAGTCCA |
| | | C |
| 287 | 557_Human_diMut_TG16CT_n1 | GGGGGAGGCTGCTGGCTAATATTAACCAAGGTCACCCCAGTTATCGGAGGAGCAAACAGGGGCTAAGTCCA |
| | | C |
| 288 | 558_Human_diMut_TG16GA_n1 | GGGGGAGGCTGCTGGGAAATATTAACCAAGGTCACCCCAGTTATCGGAGGAGCAAACAGGGGCTAAGTCCA |
| | | C |
| 289 | 561_Human_diMut_GA17AC_n1 | GGGGGAGGCTGCTGGTACATATTAACCAAGGTCACCCCAGTTATCGGAGGAGCAAACAGGGGCTAAGTCCA |
| | | C |
| 290 | 563_Human_diMut_GA17AT_n1 | GGGGGAGGCTGCTGGTATATATTAACCAAGGTCACCCCAGTTATCGGAGGAGCAAACAGGGGCTAAGTCCA |
| | | C |
| 291 | 565_Human_diMut_GA17CG_n1 | GGGGGAGGCTGCTGGTCGATATTAACCAAGGTCACCCCAGTTATCGGAGGAGCAAACAGGGGCTAAGTCCA |
| | | C |
| 292 | 568_Human_diMut_GA17TG_n1 | GGGGGAGGCTGCTGGTTGATATTAACCAAGGTCACCCCAGTTATCGGAGGAGCAAACAGGGGCTAAGTCCA |
| | | C |
| 293 | 572_Human_diMut_AA18CT_n1 | GGGGGAGGCTGCTGGTGCTTATTAACCAAGGTCACCCCAGTTATCGGAGGAGCAAACAGGGGCTAAGTCCA |
| | | C |
| 294 | 578_Human_diMut_AA18TT_n1 | GGGGGAGGCTGCTGGTGTTTATTAACCAAGGTCACCCCAGTTATCGGAGGAGCAAACAGGGGCTAAGTCCA |
| | | C |
| 295 | 583_Human_diMut_AT19GC_n1 | GGGGGAGGCTGCTGGTGAGCATTAACCAAGGTCACCCCAGTTATCGGAGGAGCAAACAGGGGCTAAGTCCA |
| | | C |
| 296 | 584_Human_diMut_AT19GG_n1 | GGGGGAGGCTGCTGGTGAGGATTAACCAAGGTCACCCCAGTTATCGGAGGAGCAAACAGGGGCTAAGTCCA |
| | | C |
| 297 | 585_Human_diMut_AT19TA_n1 | GGGGGAGGCTGCTGGTGATAATTAACCAAGGTCACCCCAGTTATCGGAGGAGCAAACAGGGGCTAAGTCCA |
| | | C |
| 298 | 586_Human_diMut_AT19TC_n1 | GGGGGAGGCTGCTGGTGATCATTAACCAAGGTCACCCCAGTTATCGGAGGAGCAAACAGGGGCTAAGTCCA |
| | | C |
| 299 | 587_Human_diMut_AT19TG_n1 | GGGGGAGGCTGCTGGTGATGATTAACCAAGGTCACCCCAGTTATCGGAGGAGCAAACAGGGGCTAAGTCCA |
| | | C |
| 300 | 589_Human_diMut_TA20AG_n1 | GGGGGAGGCTGCTGGTGAAAGTTAACCAAGGTCACCCCAGTTATCGGAGGAGCAAACAGGGGCTAAGTCCA |
| | | C |
| 301 | 591_Human_diMut_TA20CC_n1 | GGGGGAGGCTGCTGGTGAACCTTAACCAAGGTCACCCCAGTTATCGGAGGAGCAAACAGGGGCTAAGTCCA |
| | | C |
| 302 | 592_Human_diMut_TA20CG_n1 | GGGGGAGGCTGCTGGTGAACGTTAACCAAGGTCACCCCAGTTATCGGAGGAGCAAACAGGGGCTAAGTCCA |
| | | C |
| 303 | 593_Human_diMut_TA20CT_n1 | GGGGGAGGCTGCTGGTGAACTTTAACCAAGGTCACCCCAGTTATCGGAGGAGCAAACAGGGGCTAAGTCCA |
| | | C |
| 304 | 596_Human_diMut_TA20GT_n1 | GGGGGAGGCTGCTGGTGAAGTTTAACCAAGGTCACCCCAGTTATCGGAGGAGCAAACAGGGGCTAAGTCCA |
| | | C |
| 305 | 597_Human_diMut_AT21CA_n1 | GGGGGAGGCTGCTGGTGAATCATAACCAAGGTCACCCCAGTTATCGGAGGAGCAAACAGGGGCTAAGTCCA |
| | | C |
| 306 | 601_Human_diMut_AT21GC_n1 | GGGGGAGGCTGCTGGTGAATGCTAACCAAGGTCACCCCAGTTATCGGAGGAGCAAACAGGGGCTAAGTCCA |
| | | C |
| 308 | 602_Human_diMut_AT21GG_n1 | GGGGGAGGCTGCTGGTGAATGGTAACCAAGGTCACCCCAGTTATCGGAGGAGCAAACAGGGGCTAAGTCCA |
| | | C |
| 309 | 604_Human_diMut_AT21TC_n1 | GGGGGAGGCTGCTGGTGAATTCTAACCAAGGTCACCCCAGTTATCGGAGGAGCAAACAGGGGCTAAGTCCA |
| | | C |
| 310 | 608_Human_diMut_TT22AG_n1 | GGGGGAGGCTGCTGGTGAATAAGAACCAAGGTCACCCCAGTTATCGGAGGAGCAAACAGGGGCTAAGTCCA |
| | | C |
| 311 | 614_Human_diMut_TT22GG_n1 | GGGGGAGGCTGCTGGTGAATAGGAACCAAGGTCACCCCAGTTATCGGAGGAGCAAACAGGGGCTAAGTCCA |
| | | C |
| 312 | 616_Human_diMut_TA23AG_n1 | GGGGGAGGCTGCTGGTGAATATAGACCAAGGTCACCCCAGTTATCGGAGGAGCAAACAGGGGCTAAGTCCA |
| | | C |
| 313 | 619_Human_diMut_TA23CG_n1 | GGGGGAGGCTGCTGGTGAATATCGACCAAGGTCACCCCAGTTATCGGAGGAGCAAACAGGGGCTAAGTCCA |
| | | C |
| 314 | 621_Human_diMut_TA23GC_n1 | GGGGGAGGCTGCTGGTGAATATGCACCAAGGTCACCCCAGTTATCGGAGGAGCAAACAGGGGCTAAGTCCA |
| | | C |
| 315 | 623_Human_diMut_TA23GT_n1 | GGGGGAGGCTGCTGGTGAATATGTACCAAGGTCACCCCAGTTATCGGAGGAGCAAACAGGGGCTAAGTCCA |
| | | C |
| 316 | 624_Human_diMut_AA24CC_n1 | GGGGGAGGCTGCTGGTGAATATTCCCCAAGGTCACCCCAGTTATCGGAGGAGCAAACAGGGGCTAAGTCCA |
| | | C |
| 317 | 629_Human_diMut_AA24GT_n1 | GGGGGAGGCTGCTGGTGAATATTGTCCAAGGTCACCCCAGTTATCGGAGGAGCAAACAGGGGCTAAGTCCA |
| | | C |
| 318 | 650_Human_diMut_CC26TT_n1 | GGGGGAGGCTGCTGGTGAATATTAATTAAGGTCACCCCAGTTATCGGAGGAGCAAACAGGGGCTAAGTCCA |
| | | C |
| 319 | 667_Human_diMut_AA28TG_n1 | GGGGGAGGCTGCTGGTGAATATTAACCTGGGTCACCCCAGTTATCGGAGGAGCAAACAGGGGCTAAGTCCA |
| | | C |
| 320 | 678_Human_diMut_GG30AA_n1 | GGGGGAGGCTGCTGGTGAATATTAACCAAAATCACCCCAGTTATCGGAGGAGCAAACAGGGGCTAAGTCCA |
| | | C |
| 321 | 679_Human_diMut_GG30AC_n1 | GGGGGAGGCTGCTGGTGAATATTAACCAAACTCACCCCAGTTATCGGAGGAGCAAACAGGGGCTAAGTCCA |
| | | C |
| 322 | 682_Human_diMut_GG30CC_n1 | GGGGGAGGCTGCTGGTGAATATTAACCAACCTCACCCCAGTTATCGGAGGAGCAAACAGGGGCTAAGTCCA |
| | | C |
| 323 | 688_Human_diMut_GT31AC_n1 | GGGGGAGGCTGCTGGTGAATATTAACCAAGACCACCCCAGTTATCGGAGGAGCAAACAGGGGCTAAGTCCA |
| | | C |
| 324 | 689_Human_diMut_GT31AG_n1 | GGGGGAGGCTGCTGGTGAATATTAACCAAGAGCACCCCAGTTATCGGAGGAGCAAACAGGGGCTAAGTCCA |
| | | C |
| 325 | 694_Human_diMut_GT31TC_n1 | GGGGGAGGCTGCTGGTGAATATTAACCAAGTCCACCCCAGTTATCGGAGGAGCAAACAGGGGCTAAGTCCA |
| | | C |
| 326 | 698_Human_diMut_TC32AT_n1 | GGGGGAGGCTGCTGGTGAATATTAACCAAGGATACCCCAGTTATCGGAGGAGCAAACAGGGGCTAAGTCCA |
| | | C |
| 327 | 704_Human_diMut_TC32GT_n1 | GGGGGAGGCTGCTGGTGAATATTAACCAAGGGTACCCCAGTTATCGGAGGAGCAAACAGGGGCTAAGTCCA |
| | | C |
| 328 | 705_Human_diMut_CA33AC_n1 | GGGGGAGGCTGCTGGTGAATATTAACCAAGGTACCCCCAGTTATCGGAGGAGCAAACAGGGGCTAAGTCCA |
| | | C |
| 329 | 706_Human_diMut_CA33AG_n1 | GGGGGAGGCTGCTGGTGAATATTAACCAAGGTAGCCCCAGTTATCGGAGGAGCAAACAGGGGCTAAGTCCA |
| | | C |
| 330 | 709_Human_diMut_CA33GG_n1 | GGGGGAGGCTGCTGGTGAATATTAACCAAGGTGGCCCCAGTTATCGGAGGAGCAAACAGGGGCTAAGTCCA |
| | | C |
| 331 | 720_Human_diMut_AC34TA_n1 | GGGGGAGGCTGCTGGTGAATATTAACCAAGGTCTACCCAGTTATCGGAGGAGCAAACAGGGGCTAAGTCCA |
| | | C |
| 332 | 723_Human_diMut_CC35AA_n1 | GGGGGAGGCTGCTGGTGAATATTAACCAAGGTCAAACCAGTTATCGGAGGAGCAAACAGGGGCTAAGTCCA |
| | | C |
| 323 | 728_Human_diMut_CC35GT_n1 | GGGGGAGGCTGCTGGTGAATATTAACCAAGGTCAGTCCAGTTATCGGAGGAGCAAACAGGGGCTAAGTCCA |
| | | C |
| 324 | 733_Human_diMut_CC36AG_n1 | GGGGGAGGCTGCTGGTGAATATTAACCAAGGTCACAGCAGTTATCGGAGGAGCAAACAGGGGCTAAGTCCA |
| | | C |
| 325 | 734_Human_diMut_CC36AT_n1 | GGGGGAGGCTGCTGGTGAATATTAACCAAGGTCACATCAGTTATCGGAGGAGCAAACAGGGGCTAAGTCCA |
| | | C |
| 326 | 736_Human_diMut_CC36GG_n1 | GGGGGAGGCTGCTGGTGAATATTAACCAAGGTCACGGCAGTTATCGGAGGAGCAAACAGGGGCTAAGTCCA |
| | | C |
| 327 | 737_Human_diMut_CC36GT_n1 | GGGGGAGGCTGCTGGTGAATATTAACCAAGGTCACGTCAGTTATCGGAGGAGCAAACAGGGGCTAAGTCCA |
| | | C |
| 328 | 738_Human_diMut_CC36TA_n1 | GGGGGAGGCTGCTGGTGAATATTAACCAAGGTCACTACAGTTATCGGAGGAGCAAACAGGGGCTAAGTCCA |
| | | C |
| 329 | 739_Human_diMut_CC36TG_n1 | GGGGGAGGCTGCTGGTGAATATTAACCAAGGTCACTGCAGTTATCGGAGGAGCAAACAGGGGCTAAGTCCA |
| | | C |
| 340 | 740_Human_diMut_CC36TT_n1 | GGGGGAGGCTGCTGGTGAATATTAACCAAGGTCACTTCAGITATCGGAGGAGCAAACAGGGGCTAAGTCCA |
| | | C |
| 341 | 741_Human_diMut_CC37AA_n1 | GGGGGAGGCTGCTGGTGAATATTAACCAAGGTCACCAAAGTTATCGGAGGAGCAAACAGGGGCTAAGTCCA |
| | | C |
| 342 | 743_Human_diMut_CC37AT_n1 | GGGGGAGGCTGCTGGTGAATATTAACCAAGGTCACCATAGTTATCGGAGGAGCAAACAGGGGCTAAGTCCA |
| | | C |
| 343 | 746_Human_diMut_CC37GT_n1 | GGGGGAGGCTGCTGGTGAATATTAACCAAGGTCACCGTAGTTATCGGAGGAGCAAACAGGGGCTAAGTCCA |
| | | C |
| 344 | 747_Human_diMut_CC37TA_n1 | GGGGGAGGCTGCTGGTGAATATTAACCAAGGTCACCTAAGTTATCGGAGGAGCAAACAGGGGCTAAGTCCA |
| | | C |
| 345 | 748_Human_diMut_CC37TG_n1 | GGGGGAGGCTGCTGGTGAATATTAACCAAGGTCACCTGAGTTATCGGAGGAGCAAACAGGGGCTAAGTCCA |
| | | C |
| 346 | 750_Human_diMut_CA38AC_n1 | GGGGGAGGCTGCTGGTGAATATTAACCAAGGTCACCCACGTTATCGGAGGAGCAAACAGGGGCTAAGTCCA |
| | | C |
| 347 | 752_Human_diMut_CA38AT_n1 | GGGGGAGGCTGCTGGTGAATATTAACCAAGGTCACCCATGTTATCGGAGGAGCAAACAGGGGCTAAGTCCA |
| | | C |
| 348 | 753_Human_diMut_CA38GC_n1 | GGGGGAGGCTGCTGGTGAATATTAACCAAGGTCACCCGCGTTATCGGAGGAGCAAACAGGGGCTAAGTCCA |
| | | C |
| 349 | 754_Human_diMut_CA38GG_n1 | GGGGGAGGCTGCTGGTGAATATTAACCAAGGTCACCCGGGTTATCGGAGGAGCAAACAGGGGCTAAGTCCA |
| | | C |
| 350 | 755_Human_diMut_CA38GT_n1 | GGGGGAGGCTGCTGGTGAATATTAACCAAGGTCACCCGTGTTATCGGAGGAGCAAACAGGGGCTAAGTCCA |
| | | C |
| 351 | 756_Human_diMut_CA38TC_n1 | GGGGGAGGCTGCTGGTGAATATTAACCAAGGTCACCCTCGTTATCGGAGGAGCAAACAGGGGCTAAGTCCA |
| | | C |
| 352 | 757_Human_diMut_CA38TG_n1 | GGGGGAGGCTGCTGGTGAATATTAACCAAGGTCACCCTGGTTATCGGAGGAGCAAACAGGGGCTAAGTCCA |
| | | C |
| 353 | 758_Human_diMut_CA38TT_n1 | GGGGGAGGCTGCTGGTGAATATTAACCAAGGTCACCCTTGTTATCGGAGGAGCAAACAGGGGCTAAGTCCA |
| | | C |
| 354 | 759_Human_diMut_AG39CA_n1 | GGGGGAGGCTGCTGGTGAATATTAACCAAGGTCACCCCCATTATCGGAGGAGCAAACAGGGGCTAAGTCCA |
| | | C |
| 355 | 765_Human_diMut_AG39TA_n1 | GGGGGAGGCTGCTGGTGAATATTAACCAAGGTCACCCCTATTATCGGAGGAGCAAACAGGGGCTAAGTCCA |
| | | C |
| 356 | 766_Human_diMut_AG39TC_n1 | GGGGGAGGCTGCTGGTGAATATTAACCAAGGTCACCCCTCTTATCGGAGGAGCAAACAGGGGCTAAGTCCA |
| | | C |
| 357 | 767_Human_diMut_AG39TT_n1 | GGGGGAGGCTGCTGGTGAATATTAACCAAGGTCACCCCTTTTATCGGAGGAGCAAACAGGGGCTAAGTCCA |
| | | C |
| 358 | 772_Human_diMut_GT40CC_n1 | GGGGGAGGCTGCTGGTGAATATTAACCAAGGTCACCCCACCTATCGGAGGAGCAAACAGGGGCTAAGTCCA |
| | | C |
| 359 | 776_Human_diMut_GT40TG_n1 | GGGGGAGGCTGCTGGTGAATATTAACCAAGGTCACCCCATGTATCGGAGGAGCAAACAGGGGCTAAGTCCA |
| | | C |
| 360 | 777_Human_diMut_TT41AA_n1 | GGGGGAGGCTGCTGGTGAATATTAACCAAGGTCACCCCAGAAATCGGAGGAGCAAACAGGGGCTAAGTCCA |
| | | C |
| 361 | 781_Human_diMut_TT41CC_n1 | GGGGGAGGCTGCTGGTGAATATTAACCAAGGTCACCCCAGCCATCGGAGGAGCAAACAGGGGCTAAGTCCA |
| | | C |
| 362 | 787_Human_diMut_TA42AG_n1 | GGGGGAGGCTGCTGGTGAATATTAACCAAGGTCACCCCAGTAGTCGGAGGAGCAAACAGGGGCTAAGTCCA |
| | | C |
| 363 | 790_Human_diMut_TA42CG_n1 | GGGGGAGGCTGCTGGTGAATATTAACCAAGGTCACCCCAGTCGTCGGAGGAGCAAACAGGGGCTAAGTCCA |
| | | C |
| 364 | 794_Human_diMut_TA42GT_n1 | GGGGGAGGCTGCTGGTGAATATTAACCAAGGTCACCCCAGTGTTCGGAGGAGCAAACAGGGGCTAAGTCCA |
| | | C |
| 365 | 795_Human_diMut_AT43CA_n1 | GGGGGAGGCTGCTGGTGAATATTAACCAAGGTCACCCCAGTTCACGGAGGAGCAAACAGGGGCTAAGTCCA |
| | | C |
| 366 | 799_Human_diMut_AT43GC_n1 | GGGGGAGGCTGCTGGTGAATATTAACCAAGGTCACCCCAGTTGCCGGAGGAGCAAACAGGGGCTAAGTCCA |
| | | C |
| 367 | 802_Human_diMut_AT43TC_n1 | GGGGGAGGCTGCTGGTGAATATTAACCAAGGTCACCCCAGTTTCCGGAGGAGCAAACAGGGGCTAAGTCCA |
| | | C |
| 368 | 806_Human_diMut_TC44AT_n1 | GGGGGAGGCTGCTGGTGAATATTAACCAAGGTCACCCCAGTTAATGGAGGAGCAAACAGGGGCTAAGTCCA |
| | | C |
| 369 | 807_Human_diMut_TC44CA_n1 | GGGGGAGGCTGCTGGTGAATATTAACCAAGGTCACCCCAGTTACAGGAGGAGCAAACAGGGGCTAAGTCCA |
| | | C |
| 370 | 811_Human_diMut_TC44GG_n1 | GGGGGAGGCTGCTGGTGAATATTAACCAAGGTCACCCCAGTTAGGGGAGGAGCAAACAGGGGCTAAGTCCA |
| | | C |
| 371 | 815_Human_diMut_CG45AT_n1 | GGGGGAGGCTGCTGGTGAATATTAACCAAGGTCACCCCAGTTATATGAGGAGCAAACAGGGGCTAAGTCCA |
| | | C |
| 372 | 818_Human_diMut_CG45GT_n1 | GGGGGAGGCTGCTGGTGAATATTAACCAAGGTCACCCCAGTTATGTGAGGAGCAAACAGGGGCTAAGTCCA |
| | | C |
| 373 | 821_Human_diMut_CG45TT_n1 | GGGGGAGGCTGCTGGTGAATATTAACCAAGGTCACCCCAGTTATTTGAGGAGCAAACAGGGGCTAAGTCCA |
| | | C |
| 374 | 822_Human_diMut_GG46AA_n1 | GGGGGAGGCTGCTGGTGAATATTAACCAAGGTCACCCCAGTTATCAAAGGAGCAAACAGGGGCTAAGTCCA |
| | | C |
| 375 | 824_Human_diMut_GG46AT_n1 | GGGGGAGGCTGCTGGTGAATATTAACCAAGGTCACCCCAGTTATCATAGGAGCAAACAGGGGCTAAGTCCA |
| | | C |
| 376 | 826_Human_diMut_GG46CC_n1 | GGGGGAGGCTGCTGGTGAATATTAACCAAGGTCACCCCAGTTATCCCAGGAGCAAACAGGGGCTAAGTCCA |
| | | C |
| 377 | 827_Human_diMut_GG46CT_n1 | GGGGGAGGCTGCTGGTGAATATTAACCAAGGTCACCCCAGTTATCCTAGGAGCAAACAGGGGCTAAGTCCA |
| | | C |
| 378 | 828_Human_diMut_GG46TA_n1 | GGGGGAGGCTGCTGGTGAATATTAACCAAGGTCACCCCAGTTATCTAAGGAGCAAACAGGGGCTAAGTCCA |
| | | C |
| 379 | 830_Human_diMut_GG46TT_n1 | GGGGGAGGCTGCTGGTGAATATTAACCAAGGTCACCCCAGTTATCTTAGGAGCAAACAGGGGCTAAGTCCA |
| | | C |
| 380 | 832_Human_diMut_GA47AG_n1 | GGGGGAGGCTGCTGGTGAATATTAACCAAGGTCACCCCAGTTATCGAGGGAGCAAACAGGGGCTAAGTCCA |
| | | C |
| 381 | 833_Human_diMut_GA47AT_n1 | GGGGGAGGCTGCTGGTGAATATTAACCAAGGTCACCCCAGTTATCGATGGAGCAAACAGGGGCTAAGTCCA |
| | | C |
| 382 | 834_Human_diMut_GA47CC_n1 | GGGGGAGGCTGCTGGTGAATATTAACCAAGGTCACCCCAGTTATCGCCGGAGCAAACAGGGGCTAAGTCCA |
| | | C |
| 383 | 836_Human_diMut_GA47CT_n1 | GGGGGAGGCTGCTGGTGAATATTAACCAAGGTCACCCCAGTTATCGCTGGAGCAAACAGGGGCTAAGTCCA |
| | | C |
| 384 | 839_Human_diMut_GA47TT_n1 | GGGGGAGGCTGCTGGTGAATATTAACCAAGGTCACCCCAGTTATCGTTGGAGCAAACAGGGGCTAAGTCCA |
| | | C |
| 385 | 840_Human_diMut_AG48CA_n1 | GGGGGAGGCTGCTGGTGAATATTAACCAAGGTCACCCCAGTTATCGGCAGAGCAAACAGGGGCTAAGTCCA |
| | | C |
| 386 | 842_Human_diMut_AG48CT_n1 | GGGGGAGGCTGCTGGTGAATATTAACCAAGGTCACCCCAGTTATCGGCTGAGCAAACAGGGGCTAAGTCCA |
| | | C |
| 387 | 843_Human_diMut_AG48GA_n1 | GGGGGAGGCTGCTGGTGAATATTAACCAAGGTCACCCCAGTTATCGGGAGAGCAAACAGGGGCTAAGTCCA |
| | | C |
| 388 | 845_Human_diMut_AG48GT_n1 | GGGGGAGGCTGCTGGTGAATATTAACCAAGGTCACCCCAGTTATCGGGTGAGCAAACAGGGGCTAAGTCCA |
| | | C |
| 389 | 846_Human_diMut_AG48TA_n1 | GGGGGAGGCTGCTGGTGAATATTAACCAAGGTCACCCCAGTTATCGGTAGAGCAAACAGGGGCTAAGTCCA |
| | | C |
| 390 | 848_Human_diMut_AG48TT_n1 | GGGGGAGGCTGCTGGTGAATATTAACCAAGGTCACCCCAGTTATCGGTTGAGCAAACAGGGGCTAAGTCCA |
| | | C |
| 391 | 849_Human_diMut_GG49AA_n1 | GGGGGAGGCTGCTGGTGAATATTAACCAAGGTCACCCCAGTTATCGGAAAAGCAAACAGGGGCTAAGTCCA |
| | | C |
| 392 | 850_Human_diMut_GG49AC_n1 | GGGGGAGGCTGCTGGTGAATATTAACCAAGGTCACCCCAGTTATCGGAACAGCAAACAGGGGCTAAGTCCA |
| | | C |
| 393 | 851_Human_diMut_GG49AT_n1 | GGGGGAGGCTGCTGGTGAATATTAACCAAGGTCACCCCAGTTATCGGAATAGCAAACAGGGGCTAAGTCCA |
| | | C |
| 394 | 852_Human_diMut_GG49CA_n1 | GGGGGAGGCTGCTGGTGAATATTAACCAAGGTCACCCCAGTTATCGGACAAGCAAACAGGGGCTAAGTCCA |
| | | C |
| 395 | 853_Human_diMut_GG49CC_n1 | GGGGGAGGCTGCTGGTGAATATTAACCAAGGTCACCCCAGTTATCGGACCAGCAAACAGGGGCTAAGTCCA |
| | | C |
| 396 | 854_Human_diMut_GG49CT_n1 | GGGGGAGGCTGCTGGTGAATATTAACCAAGGTCACCCCAGTTATCGGACTAGCAAACAGGGGCTAAGTCCA |
| | | C |
| 397 | 855_Human_diMut_GG49TA_n1 | GGGGGAGGCTGCTGGTGAATATTAACCAAGGTCACCCCAGTTATCGGATAAGCAAACAGGGGCTAAGTCCA |
| | | C |
| 398 | 856_Human_diMut_GG49TC_n1 | GGGGGAGGCTGCTGGTGAATATTAACCAAGGTCACCCCAGTTATCGGATCAGCAAACAGGGGCTAAGTCCA |
| | | C |
| 399 | 857_Human_diMut_GG49TT_n1 | GGGGGAGGCTGCTGGTGAATATTAACCAAGGTCACCCCAGTTATCGGATTAGCAAACAGGGGCTAAGTCCA |
| | | C |
| 400 | 859_Human_diMut_GA50AG_n1 | GGGGGAGGCTGCTGGTGAATATTAACCAAGGTCACCCCAGTTATCGGAGAGGCAAACAGGGGCTAAGTCCA |
| | | C |
| 401 | 866_Human_diMut_GA50TT_n1 | GGGGGAGGCTGCTGGTGAATATTAACCAAGGTCACCCCAGTTATCGGAGTTGCAAACAGGGGCTAAGTCCA |
| | | C |
| 402 | 867_Human_diMut_AG51CA_n1 | GGGGGAGGCTGCTGGTGAATATTAACCAAGGTCACCCCAGTTATCGGAGGCACAAACAGGGGCTAAGTCCA |
| | | C |
| 403 | 868_Human_diMut_AG51CC_n1 | GGGGGAGGCTGCTGGTGAATATTAACCAAGGTCACCCCAGTTATCGGAGGCCCAAACAGGGGCTAAGTCCA |
| | | C |
| 404 | 876_Human_diMut_GC52AA_n1 | GGGGGAGGCTGCTGGTGAATATTAACCAAGGTCACCCCAGTTATCGGAGGAAAAAACAGGGGCTAAGTCCA |
| | | C |
| 405 | 881_Human_diMut_GC52CT_n1 | GGGGGAGGCTGCTGGTGAATATTAACCAAGGTCACCCCAGTTATCGGAGGACTAAACAGGGGCTAAGTCCA |
| | | C |
| 406 | 885_Human_diMut_CA53AC_n1 | GGGGGAGGCTGCTGGTGAATATTAACCAAGGTCACCCCAGTTATCGGAGGAGACAACAGGGGCTAAGTCCA |
| | | C |
| 407 | 886_Human_diMut_CA53AG_n1 | GGGGGAGGCTGCTGGTGAATATTAACCAAGGTCACCCCAGTTATCGGAGGAGAGAACAGGGGCTAAGTCCA |
| | | C |
| 408 | 887_Human_diMut_CA53AT_n1 | GGGGGAGGCTGCTGGTGAATATTAACCAAGGTCACCCCAGTTATCGGAGGAGATAACAGGGGCTAAGTCCA |
| | | C |
| 409 | 891_Human_diMut_CA53TC_n1 | GGGGGAGGCTGCTGGTGAATATTAACCAAGGTCACCCCAGTTATCGGAGGAGTCAACAGGGGCTAAGTCCA |
| | | C |
| 410 | 892_Human_diMut_CA53TG_n1 | GGGGGAGGCTGCTGGTGAATATTAACCAAGGTCACCCCAGTTATCGGAGGAGTGAACAGGGGCTAAGTCCA |
| | | C |
| 411 | 898_Human_diMut_AA54GG_n1 | GGGGGAGGCTGCTGGTGAATATTAACCAAGGTCACCCCAGTTATCGGAGGAGCGGACAGGGGCTAAGTCCA |
| | | C |
| 412 | 900_Human_diMut_AA54TC_n1 | GGGGGAGGCTGCTGGTGAATATTAACCAAGGTCACCCCAGTTATCGGAGGAGCTCACAGGGGCTAAGTCCA |
| | | C |
| 413 | 906_Human_diMut_AA55GC_n1 | GGGGGAGGCTGCTGGTGAATATTAACCAAGGTCACCCCAGTTATCGGAGGAGCAGCCAGGGGCTAAGTCCA |
| | | C |
| 414 | 933_Human_diMut_AG58GA_n1 | GGGGGAGGCTGCTGGTGAATATTAACCAAGGTCACCCCAGTTATCGGAGGAGCAAACGAGGGCTAAGTCCA |
| | | C |
| 415 | 941_Human_diMut_GG59AT_n1 | GGGGGAGGCTGCTGGTGAATATTAACCAAGGTCACCCCAGTTATCGGAGGAGCAAACAATGGCTAAGTCCA |
| | | C |
| 416 | 943_Human_diMut_GG59CC_n1 | GGGGGAGGCTGCTGGTGAATATTAACCAAGGTCACCCCAGTTATCGGAGGAGCAAACACCGGCTAAGTCCA |
| | | C |
| 417 | 947_Human_diMut_GG59TT_n1 | GGGGGAGGCTGCTGGTGAATATTAACCAAGGTCACCCCAGTTATCGGAGGAGCAAACATTGGCTAAGTCCA |
| | | C |
| 418 | 950_Human_diMut_GG60AT_n1 | GGGGGAGGCTGCTGGTGAATATTAACCAAGGTCACCCCAGTTATCGGAGGAGCAAACAGATGCTAAGTCCA |
| | | C |
| 419 | 955_Human_diMut_GG60TC_n1 | GGGGGAGGCTGCTGGTGAATATTAACCAAGGTCACCCCAGTTATCGGAGGAGCAAACAGTCGCTAAGTCCA |
| | | C |
| 420 | 957_Human_diMut_GG61AA_n1 | GGGGGAGGCTGCTGGTGAATATTAACCAAGGTCACCCCAGTTATCGGAGGAGCAAACAGGAACTAAGTCCA |
| | | C |
| 421 | 958_Human_diMut_GG61AC_n1 | GGGGGAGGCTGCTGGTGAATATTAACCAAGGTCACCCCAGTTATCGGAGGAGCAAACAGGACCTAAGTCCA |
| | | C |
| 422 | 960_Human_diMut_GG61CA_n1 | GGGGGAGGCTGCTGGTGAATATTAACCAAGGTCACCCCAGTTATCGGAGGAGCAAACAGGCACTAAGTCCA |
| | | C |
| 423 | 964_Human_diMut_GG61TC_n1 | GGGGGAGGCTGCTGGTGAATATTAACCAAGGTCACCCCAGTTATCGGAGGAGCAAACAGGTCCTAAGTCCA |
| | | C |
| 424 | 965_Human_diMut_GG61TT_n1 | GGGGGAGGCTGCTGGTGAATATTAACCAAGGTCACCCCAGTTATCGGAGGAGCAAACAGGTTCTAAGTCCA |
| | | C |
| 425 | 968_Human_diMut_GC62AT_n1 | GGGGGAGGCTGCTGGTGAATATTAACCAAGGTCACCCCAGTTATCGGAGGAGCAAACAGGGATTAAGTCCA |
| | | C |
| 426 | 969_Human_diMut_GC62CA_n1 | GGGGGAGGCTGCTGGTGAATATTAACCAAGGTCACCCCAGTTATCGGAGGAGCAAACAGGGCATAAGTCCA |
| | | C |
| 427 | 980_Human_diMut_CT63GG_n1 | GGGGGAGGCTGCTGGTGAATATTAACCAAGGTCACCCCAGTTATCGGAGGAGCAAACAGGGGGGAAGTCCA |
| | | C |
| 428 | 981_Human_diMut_CT63TA_n1 | GGGGGAGGCTGCTGGTGAATATTAACCAAGGTCACCCCAGTTATCGGAGGAGCAAACAGGGGTAAAGTCCA |
| | | C |
| 429 | 983_Human_diMut_CT63TG_n1 | GGGGGAGGCTGCTGGTGAATATTAACCAAGGTCACCCCAGTTATCGGAGGAGCAAACAGGGGTGAAGTCCA |
| | | C |
| 430 | 985_Human_diMut_TA64AG_n1 | GGGGGAGGCTGCTGGTGAATATTAACCAAGGTCACCCCAGTTATCGGAGGAGCAAACAGGGGCAGAGTCCA |
| | | C |
| 431 | 986_Human_diMut_TA64AT_n1 | GGGGGAGGCTGCTGGTGAATATTAACCAAGGTCACCCCAGTTATCGGAGGAGCAAACAGGGGCATAGTCCA |
| | | C |
| 432 | 994_Human_diMut_AA65CG_n1 | GGGGGAGGCTGCTGGTGAATATTAACCAAGGTCACCCCAGTTATCGGAGGAGCAAACAGGGGCTCGGTCCA |
| | | C |
| 433 | 1022_Human_diMut_TC68AT_n1 | GGGGGAGGCTGCTGGTGAATATTAACCAAGGTCACCCCAGTTATCGGAGGAGCAAACAGGGGCTAAGATCA |
| | | C |
| 434 | 1023_Human_diMut_TC68CA_n1 | GGGGGAGGCTGCTGGTGAATATTAACCAAGGTCACCCCAGTTATCGGAGGAGCAAACAGGGGCTAAGCACA |
| | | C |
| 435 | 1028_Human_diMut_TC68GT_n1 | GGGGGAGGCTGCTGGTGAATATTAACCAAGGTCACCCCAGTTATCGGAGGAGCAAACAGGGGCTAAGGTCA |
| | | C |
| 436 | 1029_Human_diMut_CC69AA_n1 | GGGGGAGGCTGCTGGTGAATATTAACCAAGGTCACCCCAGTTATCGGAGGAGCAAACAGGGGCTAAGTAAA |
| | | C |
| 437 | 4267_Human_quadMut_1T_3A_8T_48T_n1 | TGAGGAGTCTGCTGGTGAATATTAACCAAGGTCACCCCAGTTATCGGTGGAGCAAACAGGGGCTAAGTCCA |
| | | C |
| 438 | 4268_Human_quadMut_1T_3A_8C_52T_n1 | TGAGGAGCCTGCTGGTGAATATTAACCAAGGTCACCCCAGTTATCGGAGGATCAAACAGGGGCTAAGTCCA |
| | | C |
| 439 | 4269_Human_quadMut_1A_3A_8A_62C_n1 | AGAGGAGACTGCTGGTGAATATTAACCAAGGTCACCCCAGTTATCGGAGGAGCAAACAGGGCCTAAGTCCA |
| | | C |
| 440 | 4272_Human_quadMut_1A_3T_14A_37T_n1 | AGTGGAGGCTGCTAGTGAATATTAACCAAGGTCACCTCAGTTATCGGAGGAGCAAACAGGGGCTAAGTCCA |
| | | C |
| 441 | 4282_Human_quadMut_1T_3A_35A_48G_n1 | TGAGGAGGCTGCTGGTGAATATTAACCAAGGTCAACCCAGTTATCGGGGGAGCAAACAGGGGCTAAGTCCA |
| | | C |
| 442 | 4284_Human_quadMut_1A_3T_35T_62A_n1 | AGTGGAGGCTGCTGGTGAATATTAACCAAGGTCATCCCAGTTATCGGAGGAGCAAACAGGGACTAAGTCCA |
| | | C |
| 443 | 4287_Human_quadMut_1A_3T_37A_46T_n1 | AGTGGAGGCTGCTGGTGAATATTAACCAAGGTCACCACAGTTATCTGAGGAGCAAACAGGGGCTAAGTCCA |
| | | C |
| 444 | 4288_Human_quadMut_1A_3T_37T_48T_n1 | AGTGGAGGCTGCTGGTGAATATTAACCAAGGTCACCTCAGTTATCGGTGGAGCAAACAGGGGCTAAGTCCA |
| | | C |
| 445 | 4289_Human_quadMut_1A_3A_37T_52T_n1 | AGAGGAGGCTGCTGGTGAATATTAACCAAGGTCACCTCAGTTATCGGAGGATCAAACAGGGGCTAAGTCCA |
| | | C |
| 446 | 4293_Human_quadMut_1A_3A_45T_48T_n1 | AGAGGAGGCTGCTGGTGAATATTAACCAAGGTCACCCCAGTTATTGGTGGAGCAAACAGGGGCTAAGTCCA |
| | | C |
| 447 | 4298_Human_quadMut_1T_3T_46T_52T_n1 | TGTGGAGGCTGCTGGTGAATATTAACCAAGGTCACCCCAGTTATCTGAGGATCAAACAGGGGCTAAGTCCA |
| | | C |
| 448 | 4299_Human_quadMut_1T_3T_46T_62A_n1 | TGTGGAGGCTGCTGGTGAATATTAACCAAGGTCACCCCAGTTATCTGAGGAGCAAACAGGGACTAAGTCCA |
| | | C |
| 449 | 4310_Human_quadMut_1T_8T_14T_46T_n1 | TGGGGAGTCTGCTTGTGAATATTAACCAAGGTCACCCCAGTTATCTGAGGAGCAAACAGGGGCTAAGTCCA |
| | | C |
| 450 | 4312_Human_quadMut_1T_8C_14T_52T_n1 | TGGGGAGCCTGCTTGTGAATATTAACCAAGGTCACCCCAGTTATCGGAGGATCAAACAGGGGCTAAGTCCA |
| | | C |
| 451 | 4313_Human_quadMut_1A_8T_14T_62T_n1 | AGGGGAGTCTGCTTGTGAATATTAACCAAGGTCACCCCAGTTATCGGAGGAGCAAACAGGGTCTAAGTCCA |
| | | C |
| 452 | 4314_Human_quadMut_1A_8C_14A_72A_n1 | AGGGGAGCCTGCTAGTGAATATTAACCAAGGTCACCCCAGTTATCGGAGGAGCAAACAGGGGCTAAGTCCA |
| | | A |
| 453 | 4315_Human_quadMut_1A_8C_35T_37A_n1 | AGGGGAGCCTGCTGGTGAATATTAACCAAGGTCATCACAGTTATCGGAGGAGCAAACAGGGGCTAAGTCCA |
| | | C |
| 454 | 4317_Human_quadMut_1A_8A_35T_46A_n1 | AGGGGAGACTGCTGGTGAATATTAACCAAGGTCATCCCAGTTATCAGAGGAGCAAACAGGGGCTAAGTCCA |
| | | C |
| 455 | 4318_Human_quadMut_1T_8C_35G_48G_n1 | TGGGGAGCCTGCTGGTGAATATTAACCAAGGTCAGCCCAGTTATCGGGGGAGCAAACAGGGGCTAAGTCCA |
| | | C |
| 456 | 4320_Human_quadMut_1T_8A_35A_62C_n1 | TGGGGAGACTGCTGGTGAATATTAACCAAGGTCAACCCAGTTATCGGAGGAGCAAACAGGGCCTAAGTCCA |
| | | C |
| 457 | 4323_Human_quadMut_1A_8A_37A_46A_n1 | AGGGGAGACTGCTGGTGAATATTAACCAAGGTCACCACAGTTATCAGAGGAGCAAACAGGGGCTAAGTCCA |
| | | C |
| 458 | 4325_Human_quadMut_1T_8C_37T_52A_n1 | TGGGGAGCCTGCTGGTGAATATTAACCAAGGTCACCTCAGTTATCGGAGGAACAAACAGGGGCTAAGTCCA |
| | | C |
| 459 | 4340_Human_quadMut_1A_8T_52T_62T_n1 | AGGGGAGTCTGCTGGTGAATATTAACCAAGGTCACCCCAGTTATCGGAGGATCAAACAGGGTCTAAGTCCA |
| | | C |
| 460 | 4345_Human_quadMut_1T_14A_35G_46T_n1 | TGGGGAGGCTGCTAGTGAATATTAACCAAGGTCAGCCCAGTTATCTGAGGAGCAAACAGGGGCTAAGTCCA |
| | | C |
| 461 | 4353_Human_quadMut_1T_14T_37A_52T_n1 | TGGGGAGGCTGCTTGTGAATATTAACCAAGGTCACCACAGTTATCGGAGGATCAAACAGGGGCTAAGTCCA |
| | | C |
| 462 | 4371_Human_quadMut_1A_35T_37A_45T_n1 | AGGGGAGGCTGCTGGTGAATATTAACCAAGGTCATCACAGTTATTGGAGGAGCAAACAGGGGCTAAGTCCA |
| | | C |
| 463 | 4374_Human_quadMut_1A_35A_37A_52T_n1 | AGGGGAGGCTGCTGGTGAATATTAACCAAGGTCAACACAGTTATCGGAGGATCAAACAGGGGCTAAGTCCA |
| | | C |
| 464 | 4376_Human_quadMut_1A_35A_37A_72T_n1 | AGGGGAGGCTGCTGGTGAATATTAACCAAGGTCAACACAGTTATCGGAGGAGCAAACAGGGGCTAAGTCCA |
| | | T |
| 465 | 4377_Human_quadMut_1T_35G_45T_46T_n1 | TGGGGAGGCTGCTGGTGAATATTAACCAAGGTCAGCCCAGTTATTTGAGGAGCAAACAGGGGCTAAGTCCA |
| | | C |
| 466 | 4378_Human_quadMut_1T_35G_45T_48T_n1 | TGGGGAGGCTGCTGGTGAATATTAACCAAGGTCAGCCCAGTTATTGGTGGAGCAAACAGGGGCTAAGTCCA |
| | | C |
| 467 | 4379_Human_quadMut_1A_35G_45T_52C_n1 | AGGGGAGGCTGCTGGTGAATATTAACCAAGGTCAGCCCAGTTATTGGAGGACCAAACAGGGGCTAAGTCCA |
| | | C |
| 468 | 4381_Human_quadMut_1A_35T_45T_72A_n1 | AGGGGAGGCTGCTGGTGAATATTAACCAAGGTCATCCCAGTTATTGGAGGAGCAAACAGGGGCTAAGTCCA |
| | | A |
| 469 | 4382_Human_quadMut_1A_35G_46A_48G_n1 | AGGGGAGGCTGCTGGTGAATATTAACCAAGGTCAGCCCAGTTATCAGGGGAGCAAACAGGGGCTAAGTCCA |
| | | C |
| 470 | 4383_Human_quadMut_1A_35A_46T_52A_n1 | AGGGGAGGCTGCTGGTGAATATTAACCAAGGTCAACCCAGTTATCTGAGGAACAAACAGGGGCTAAGTCCA |
| | | C |
| 471 | 4384_Human_quadMut_1A_35T_46T_62C_n1 | AGGGGAGGCTGCTGGTGAATATTAACCAAGGTCATCCCAGTTATCTGAGGAGCAAACAGGGCCTAAGTCCA |
| | | C |
| 472 | 4387_Human_quadMut_1T_35T_48G_62T_n1 | TGGGGAGGCTGCTGGTGAATATTAACCAAGGTCATCCCAGTTATCGGGGGAGCAAACAGGGTCTAAGTCCA |
| | | C |
| 473 | 4388_Human_quadMut_1A_35T_48G_72A_n1 | AGGGGAGGCTGCTGGTGAATATTAACCAAGGTCATCCCAGTTATCGGGGGAGCAAACAGGGGCTAAGTCCA |
| | | A |
| 474 | 4394_Human_quadMut_1A_37A_45G_52T_n1 | AGGGGAGGCTGCTGGTGAATATTAACCAAGGTCACCACAGTTATGGGAGGATCAAACAGGGGCTAAGTCCA |
| | | C |
| 475 | 4395_Human_quadMut_1T_37T_45G_62A_n1 | TGGGGAGGCTGCTGGTGAATATTAACCAAGGTCACCTCAGTTATGGGAGGAGCAAACAGGGACTAAGTCCA |
| | | C |
| 476 | 4396_Human_quadMut_1A_37T_45G_72A_n1 | AGGGGAGGCTGCTGGTGAATATTAACCAAGGTCACCTCAGTTATGGGAGGAGCAAACAGGGGCTAAGTCCA |
| | | A |
| 477 | 4398_Human_quadMut_1T_37A_46A_52C_n1 | TGGGGAGGCTGCTGGTGAATATTAACCAAGGTCACCACAGTTATCAGAGGACCAAACAGGGGCTAAGTCCA |
| | | C |
| 478 | 4401_Human_quadMut_1A_37A_48G_52C_n1 | AGGGGAGGCTGCTGGTGAATATTAACCAAGGTCACCACAGTTATCGGGGGACCAAACAGGGGCTAAGTCCA |
| | | C |
| 479 | 4402_Human_quadMut_1T_37T_48G_62T_n1 | TGGGGAGGCTGCTGGTGAATATTAACCAAGGTCACCTCAGTTATCGGGGGAGCAAACAGGGTCTAAGTCCA |
| | | C |
| 480 | 4405_Human_quadMut_1A_37A_52A_72T_n1 | AGGGGAGGCTGCTGGTGAATATTAACCAAGGTCACCACAGTTATCGGAGGAACAAACAGGGGCTAAGTCCA |
| | | T |
| 481 | 4407_Human_quadMut_1T_45G_46T_48G_n1 | TGGGGAGGCTGCTGGTGAATATTAACCAAGGTCACCCCAGTTATGTGGGGAGCAAACAGGGGCTAAGTCCA |
| | | C |
| 482 | 4410_Human_quadMut_1T_45G_46A_72A_n1 | TGGGGAGGCTGCTGGTGAATATTAACCAAGGTCACCCCAGTTATGAGAGGAGCAAACAGGGGCTAAGTCCA |
| | | A |
| 483 | 4416_Human_quadMut_1T_45T_62C_72A_n1 | TGGGGAGGCTGCTGGTGAATATTAACCAAGGTCACCCCAGTTATTGGAGGAGCAAACAGGGCCTAAGTCCA |
| | | A |
| 484 | 4417_Human_quadMut_1T_46A_48T_52C_n1 | TGGGGAGGCTGCTGGTGAATATTAACCAAGGTCACCCCAGTTATCAGTGGACCAAACAGGGGCTAAGTCCA |
| | | C |
| 485 | 4418_Human_quadMut_1T_46A_48T_62A_n1 | TGGGGAGGCTGCTGGTGAATATTAACCAAGGTCACCCCAGTTATCAGTGGAGCAAACAGGGACTAAGTCCA |
| | | C |
| 486 | 4419_Human_quadMut_1T_46A_48T_72A_n1 | TGGGGAGGCTGCTGGTGAATATTAACCAAGGTCACCCCAGTTATCAGTGGAGCAAACAGGGGCTAAGTCCA |
| | | A |
| 487 | 4423_Human_quadMut_1T_48G_52A_62T_n1 | TGGGGAGGCTGCTGGTGAATATTAACCAAGGTCACCCCAGTTATCGGGGGAACAAACAGGGTCTAAGTCCA |
| | | C |
| 488 | 4426_Human_quadMut_1T_52T_62C_72A_n1 | TGGGGAGGCTGCTGGTGAATATTAACCAAGGTCACCCCAGTTATCGGAGGATCAAACAGGGCCTAAGTCCA |
| | | A |
| 489 | 4435_Human_quadMut_3A_8A_35G_37T_n1 | GGAGGAGACTGCTGGTGAATATTAACCAAGGTCAGCTCAGTTATCGGAGGAGCAAACAGGGGCTAAGTCCA |
| | | C |
| 490 | 4439_Human_quadMut_3A_8T_35T_52T_n1 | GGAGGAGTCTGCTGGTGAATATTAACCAAGGTCATCCCAGTTATCGGAGGATCAAACAGGGGCTAAGTCCA |
| | | C |
| 491 | 4445_Human_quadMut_3A_8A_37A_52A_n1 | GGAGGAGACTGCTGGTGAATATTAACCAAGGTCACCACAGTTATCGGAGGAACAAACAGGGGCTAAGTCCA |
| | | C |
| 492 | 4448_Human_quadMut_3A_8A_45T_46A_n1 | GGAGGAGACTGCTGGTGAATATTAACCAAGGTCACCCCAGTTATTAGAGGAGCAAACAGGGGCTAAGTCCA |
| | | C |
| 493 | 4453_Human_quadMut_3T_8C_46T_48G_n1 | GGTGGAGCCTGCTGGTGAATATTAACCAAGGTCACCCCAGTTATCTGGGGAGCAAACAGGGGCTAAGTCCA |
| | | C |
| 494 | 4455_Human_quadMut_3A_8C_46A_62T_n1 | GGAGGAGCCTGCTGGTGAATATTAACCAAGGTCACCCCAGTTATCAGAGGAGCAAACAGGGTCTAAGTCCA |
| | | C |
| 495 | 4456_Human_quadMut_3T_8T_46A_72A_n1 | GGTGGAGTCTGCTGGTGAATATTAACCAAGGTCACCCCAGTTATCAGAGGAGCAAACAGGGGCTAAGTCCA |
| | | A |
| 496 | 4457_Human_quadMut_3T_8A_48G_52A_n1 | GGTGGAGACTGCTGGTGAATATTAACCAAGGTCACCCCAGTTATCGGGGGAACAAACAGGGGCTAAGTCCA |
| | | C |
| 497 | 4460_Human_quadMut_3T_8A_52C_62T_n1 | GGTGGAGACTGCTGGTGAATATTAACCAAGGTCACCCCAGTTATCGGAGGACCAAACAGGGTCTAAGTCCA |
| | | C |
| 498 | 4462_Human_quadMut_3T_8T_62T_72T_n1 | GGTGGAGTCTGCTGGTGAATATTAACCAAGGTCACCCCAGTTATCGGAGGAGCAAACAGGGTCTAAGTCCA |
| | | T |
| 499 | 4467_Human_quadMut_3T_14T_35G_52T_n1 | GGTGGAGGCTGCTTGTGAATATTAACCAAGGTCAGCCCAGTTATCGGAGGATCAAACAGGGGCTAAGTCCA |
| | | C |
| 500 | 4471_Human_quadMut_3T_14A_37A_46A_n1 | GGTGGAGGCTGCTAGTGAATATTAACCAAGGTCACCACAGTTATCAGAGGAGCAAACAGGGGCTAAGTCCA |
| | | C |
| 501 | 4481_Human_quadMut_3A_14T_46T_48T_n1 | GGAGGAGGCTGCTTGTGAATATTAACCAAGGTCACCCCAGTTATCTGTGGAGCAAACAGGGGCTAAGTCCA |
| | | C |
| 502 | 4482_Human_quadMut_3A_14T_46T_52C_n1 | GGAGGAGGCTGCTTGTGAATATTAACCAAGGTCACCCCAGTTATCTGAGGACCAAACAGGGGCTAAGTCCA |
| | | C |
| 503 | 4487_Human_quadMut_3T_14T_48G_72A_n1 | GGTGGAGGCTGCTTGTGAATATTAACCAAGGTCACCCCAGTTATCGGGGGAGCAAACAGGGGCTAAGTCCA |
| | | A |
| 504 | 4488_Human_quadMut_3A_14A_52C_62T_n1 | GGAGGAGGCTGCTAGTGAATATTAACCAAGGTCACCCCAGTTATCGGAGGACCAAACAGGGTCTAAGTCCA |
| | | C |
| 505 | 4492_Human_quadMut_3A_35T_37T_46T_n1 | GGAGGAGGCTGCTGGTGAATATTAACCAAGGTCATCTCAGTTATCTGAGGAGCAAACAGGGGCTAAGTCCA |
| | | C |
| 506 | 4493_Human_quadMut_3T_35T_37T_48G_n1 | GGTGGAGGCTGCTGGTGAATATTAACCAAGGTCATCTCAGTTATCGGGGGAGCAAACAGGGGCTAAGTCCA |
| | | C |
| 507 | 4494_Human_quadMut_3T_35A_37T_52C_n1 | GGTGGAGGCTGCTGGTGAATATTAACCAAGGTCAACTCAGTTATCGGAGGACCAAACAGGGGCTAAGTCCA |
| | | C |
| 508 | 4499_Human_quadMut_3T_35T_45T_52A_n1 | GGTGGAGGCTGCTGGTGAATATTAACCAAGGTCATCCCAGTTATTGGAGGAACAAACAGGGGCTAAGTCCA |
| | | C |
| 509 | 4502_Human_quadMut_3A_35G_46A_48G_n1 | GGAGGAGGCTGCTGGTGAATATTAACCAAGGTCAGCCCAGTTATCAGGGGAGCAAACAGGGGCTAAGTCCA |
| | | C |
| 510 | 4503_Human_quadMut_3T_35T_46T_52A_n1 | GGTGGAGGCTGCTGGTGAATATTAACCAAGGTCATCCCAGTTATCTGAGGAACAAACAGGGGCTAAGTCCA |
| | | C |
| 511 | 4506_Human_quadMut_3T_35G_48T_52T_n1 | GGTGGAGGCTGCTGGTGAATATTAACCAAGGTCAGCCCAGTTATCGGTGGATCAAACAGGGGCTAAGTCCA |
| | | C |
| 512 | 4507_Human_quadMut_3A_35A_48T_62T_n1 | GGAGGAGGCTGCTGGTGAATATTAACCAAGGTCAACCCAGTTATCGGTGGAGCAAACAGGGTCTAAGTCCA |
| | | C |
| 513 | 4509_Human_quadMut_3T_35T_52A_62T_n1 | GGTGGAGGCTGCTGGTGAATATTAACCAAGGTCATCCCAGTTATCGGAGGAACAAACAGGGTCTAAGTCCA |
| | | C |
| 514 | 4513_Human_quadMut_3A_37A_45T_48T_n1 | GGAGGAGGCTGCTGGTGAATATTAACCAAGGTCACCACAGTTATTGGTGGAGCAAACAGGGGCTAAGTCCA |
| | | C |
| 515 | 4519_Human_quadMut_3T_37T_46A_62A_n1 | GGTGGAGGCTGCTGGTGAATATTAACCAAGGTCACCTCAGTTATCAGAGGAGCAAACAGGGACTAAGTCCA |
| | | C |
| 516 | 4522_Human_quadMut_3A_37T_48T_62T_n1 | GGAGGAGGCTGCTGGTGAATATTAACCAAGGTCACCTCAGTTATCGGTGGAGCAAACAGGGTCTAAGTCCA |
| | | C |
| 517 | 4529_Human_quadMut_3T_45T_46T_62C_n1 | GGTGGAGGCTGCTGGTGAATATTAACCAAGGTCACCCCAGTTATTTGAGGAGCAAACAGGGCCTAAGTCCA |
| | | C |
| 518 | 4533_Human_quadMut_3A_45G_48G_72A_n1 | GGAGGAGGCTGCTGGTGAATATTAACCAAGGTCACCCCAGTTATGGGGGGAGCAAACAGGGGCTAAGTCCA |
| | | A |
| 519 | 4537_Human_quadMut_3A_46T_48T_52T_n1 | GGAGGAGGCTGCTGGTGAATATTAACCAAGGTCACCCCAGTTATCTGTGGATCAAACAGGGGCTAAGTCCA |
| | | C |
| 520 | 4539_Human_quadMut_3A_46T_48G_72G_n1 | GGAGGAGGCTGCTGGTGAATATTAACCAAGGTCACCCCAGTTATCTGGGGAGCAAACAGGGGCTAAGTCCA |
| | | G |
| 521 | 4541_Human_quadMut_3A_46T_52T_72A_n1 | GGAGGAGGCTGCTGGTGAATATTAACCAAGGTCACCCCAGTTATCTGAGGATCAAACAGGGGCTAAGTCCA |
| | | A |
| 522 | 4543_Human_quadMut_3T_48T_52A_62T_n1 | GGTGGAGGCTGCTGGTGAATATTAACCAAGGTCACCCCAGTTATCGGTGGAACAAACAGGGTCTAAGTCCA |
| | | C |
| 523 | 4545_Human_quadMut_3A_48G_62T_72A_n1 | GGAGGAGGCTGCTGGTGAATATTAACCAAGGTCACCCCAGTTATCGGGGGAGCAAACAGGGTCTAAGTCCA |
| | | A |
| 524 | 4546_Human_quadMut_3T_52T_62T_72G_n1 | GGTGGAGGCTGCTGGTGAATATTAACCAAGGTCACCCCAGTTATCGGAGGATCAAACAGGGTCTAAGTCCA |
| | | G |
| 525 | 4549_Human_quadMut_8C_14T_35T_46T_n1 | GGGGGAGCCTGCTTGTGAATATTAACCAAGGTCATCCCAGTTATCTGAGGAGCAAACAGGGGCTAAGTCCA |
| | | C |
| 526 | 4551_Human_quadMut_8T_14A_35T_52T_n1 | GGGGGAGTCTGCTAGTGAATATTAACCAAGGTCATCCCAGTTATCGGAGGATCAAACAGGGGCTAAGTCCA |
| | | C |
| 527 | 4557_Human_quadMut_8A_14A_37T_52C_n1 | GGGGGAGACTGCTAGTGAATATTAACCAAGGTCACCTCAGTTATCGGAGGACCAAACAGGGGCTAAGTCCA |
| | | C |
| 528 | 4562_Human_quadMut_8A_14T_45G_52T_n1 | GGGGGAGACTGCTTGTGAATATTAACCAAGGTCACCCCAGTTATGGGAGGATCAAACAGGGGCTAAGTCCA |
| | | C |
| 529 | 4563_Human_quadMut_8C_14A_45T_62T_n1 | GGGGGAGCCTGCTAGTGAATATTAACCAAGGTCACCCCAGTTATTGGAGGAGCAAACAGGGTCTAAGTCCA |
| | | C |
| 530 | 4566_Human_quadMut_8T_14A_46A_52A_n1 | GGGGGAGTCTGCTAGTGAATATTAACCAAGGTCACCCCAGTTATCAGAGGAACAAACAGGGGCTAAGTCCA |
| | | C |
| 531 | 4569_Human_quadMut_8A_14A_48T_52A_n1 | GGGGGAGACTGCTAGTGAATATTAACCAAGGTCACCCCAGTTATCGGTGGAACAAACAGGGGCTAAGTCCA |
| | | C |
| 532 | 4576_Human_quadMut_8C_35A_37T_46T_n1 | GGGGGAGCCTGCTGGTGAATATTAACCAAGGTCAACTCAGTTATCTGAGGAGCAAACAGGGGCTAAGTCCA |
| | | C |
| 533 | 4579_Human_quadMut_8C_35G_37A_62A_n1 | GGGGGAGCCTGCTGGTGAATATTAACCAAGGTCAGCACAGTTATCGGAGGAGCAAACAGGGACTAAGTCCA |
| | | C |
| 534 | 4580_Human_quadMut_8C_35A_37A_72A_n1 | GGGGGAGCCTGCTGGTGAATATTAACCAAGGTCAACACAGTTATCGGAGGAGCAAACAGGGGCTAAGTCCA |
| | | A |
| 535 | 4587_Human_quadMut_8A_35G_46A_52T_n1 | GGGGGAGACTGCTGGTGAATATTAACCAAGGTCAGCCCAGTTATCAGAGGATCAAACAGGGGCTAAGTCCA |
| | | C |
| 536 | 4588_Human_quadMut_8A_35T_46A_62C_n1 | GGGGGAGACTGCTGGTGAATATTAACCAAGGTCATCCCAGTTATCAGAGGAGCAAACAGGGCCTAAGTCCA |
| | | C |
| 537 | 4590_Human_quadMut_8C_35G_48T_52A_n1 | GGGGGAGCCTGCTGGTGAATATTAACCAAGGTCAGCCCAGTTATCGGTGGAACAAACAGGGGCTAAGTCCA |
| | | C |
| 538 | 4592_Human_quadMut_8C_35G_48T_72A_n1 | GGGGGAGCCTGCTGGTGAATATTAACCAAGGTCAGCCCAGTTATCGGTGGAGCAAACAGGGGCTAAGTCCA |
| | | A |
| 539 | 4599_Human_quadMut_8A_37T_45G_62T_n1 | GGGGGAGACTGCTGGTGAATATTAACCAAGGTCACCTCAGTTATGGGAGGAGCAAACAGGGTCTAAGTCCA |
| | | C |
| 540 | 4600_Human_quadMut_8T_37T_45G_72T_n1 | GGGGGAGTCTGCTGGTGAATATTAACCAAGGTCACCTCAGTTATGGGAGGAGCAAACAGGGGCTAAGTCCA |
| | | T |
| 541 | 4601_Human_quadMut_8T_37T_46T_48T_n1 | GGGGGAGTCTGCTGGTGAATATTAACCAAGGTCACCTCAGTTATCTGTGGAGCAAACAGGGGCTAAGTCCA |
| | | C |
| 542 | 4607_Human_quadMut_8T_37A_48G_72T_n1 | GGGGGAGTCTGCTGGTGAATATTAACCAAGGTCACCACAGTTATCGGGGGAGCAAACAGGGGCTAAGTCCA |
| | | T |
| 543 | 4608_Human_quadMut_8C_37T_52C_62T_n1 | GGGGGAGCCTGCTGGTGAATATTAACCAAGGTCACCTCAGTTATCGGAGGACCAAACAGGGTCTAAGTCCA |
| | | C |
| 544 | 4609_Human_quadMut_8T_37T_52T_72G_n1 | GGGGGAGTCTGCTGGTGAATATTAACCAAGGTCACCTCAGTTATCGGAGGATCAAACAGGGGCTAAGTCCA |
| | | G |
| 545 | 4616_Human_quadMut_8A_45G_48T_62A_n1 | GGGGGAGACTGCTGGTGAATATTAACCAAGGTCACCCCAGTTATGGGTGGAGCAAACAGGGACTAAGTCCA |
| | | C |
| 546 | 4619_Human_quadMut_8T_45A_52T_72G_n1 | GGGGGAGTCTGCTGGTGAATATTAACCAAGGTCACCCCAGTTATAGGAGGATCAAACAGGGGCTAAGTCCA |
| | | G |
| 547 | 4620_Human_quadMut_8A_45A_62T_72A_n1 | GGGGGAGACTGCTGGTGAATATTAACCAAGGTCACCCCAGTTATAGGAGGAGCAAACAGGGTCTAAGTCCA |
| | | A |
| 548 | 4622_Human_quadMut_8C_46A_48T_62A_n1 | GGGGGAGCCTGCTGGTGAATATTAACCAAGGTCACCCCAGTTATCAGTGGAGCAAACAGGGACTAAGTCCA |
| | | C |
| 549 | 4623_Human_quadMut_8A_46T_48T_72A_n1 | GGGGGAGACTGCTGGTGAATATTAACCAAGGTCACCCCAGTTATCTGTGGAGCAAACAGGGGCTAAGTCCA |
| | | A |
| 550 | 4624_Human_quadMut_8A_46A_52T_62C_n1 | GGGGGAGACTGCTGGTGAATATTAACCAAGGTCACCCCAGTTATCAGAGGATCAAACAGGGCCTAAGTCCA |
| | | C |
| 551 | 4626_Human_quadMut_8A_46A_62C_72T_n1 | GGGGGAGACTGCTGGTGAATATTAACCAAGGTCACCCCAGTTATCAGAGGAGCAAACAGGGCCTAAGTCCA |
| | | T |
| 552 | 4628_Human_quadMut_8C_48G_52A_72T_n1 | GGGGGAGCCTGCTGGTGAATATTAACCAAGGTCACCCCAGTTATCGGGGGAACAAACAGGGGCTAAGTCCA |
| | | T |
| 553 | 4630_Human_quadMut_8C_52T_62T_72A_n1 | GGGGGAGCCTGCTGGTGAATATTAACCAAGGTCACCCCAGTTATCGGAGGATCAAACAGGGTCTAAGTCCA |
| | | A |
| 554 | 4632_Human_quadMut_14A_35G_37A_46A_n | GGGGGAGGCTGCTAGTGAATATTAACCAAGGTCAGCACAGTTATCAGAGGAGCAAACAGGGGCTAAGTCCA |
| | | C |
| 555 | 4633_Human_quadMut_14T_35A_37T_48G_n1 | GGGGGAGGCTGCTTGTGAATATTAACCAAGGTCAACTCAGTTATCGGGGGAGCAAACAGGGGCTAAGTCCA |
| | | C |
| 556 | 4634_Human_quadMut_14A_35A_37A_52T_n1 | GGGGGAGGCTGCTAGTGAATATTAACCAAGGTCAACACAGTTATCGGAGGATCAAACAGGGGCTAAGTCCA |
| | | C |
| 557 | 4635_Human_quadMut_14A_35G_37A_62A_n1 | GGGGGAGGCTGCTAGTGAATATTAACCAAGGTCAGCACAGTTATCGGAGGAGCAAACAGGGACTAAGTCCA |
| | | C |
| 558 | 4638_Human_quadMut_14A_35G_45T_48T_n1 | GGGGGAGGCTGCTAGTGAATATTAACCAAGGTCAGCCCAGTTATTGGTGGAGCAAACAGGGGCTAAGTCCA |
| | | C |
| 559 | 4642_Human_quadMut_14T_35A_46A_48G_n1 | GGGGGAGGCTGCTTGTGAATATTAACCAAGGTCAACCCAGTTATCAGGGGAGCAAACAGGGGCTAAGTCCA |
| | | C |
| 560 | 4643_Human_quadMut_14T_35T_46T_52A_n1 | GGGGGAGGCTGCTTGTGAATATTAACCAAGGTCATCCCAGTTATCTGAGGAACAAACAGGGGCTAAGTCCA |
| | | C |
| 561 | 4644_Human_quadMut_14A_35T_46A_62A_n1 | GGGGGAGGCTGCTAGTGAATATTAACCAAGGTCATCCCAGTTATCAGAGGAGCAAACAGGGACTAAGTCCA |
| | | C |
| 562 | 4647_Human_quadMut_14T_35T_48G_62C_n1 | GGGGGAGGCTGCTTGTGAATATTAACCAAGGTCATCCCAGTTATCGGGGGAGCAAACAGGGCCTAAGTCCA |
| | | C |
| 563 | 4649_Human_quadMut_14A_35A_52T_62T_n1 | GGGGGAGGCTGCTAGTGAATATTAACCAAGGTCAACCCAGTTATCGGAGGATCAAACAGGGTCTAAGTCCA |
| | | C |
| 564 | 4654_Human_quadMut_14T_37A_45T_52A_n1 | GGGGGAGGCTGCTTGTGAATATTAACCAAGGTCACCACAGTTATTGGAGGAACAAACAGGGGCTAAGTCCA |
| | | C |
| 565 | 4659_Human_quadMut_14A_37A_46A_62T_n1 | GGGGGAGGCTGCTAGTGAATATTAACCAAGGTCACCACAGTTATCAGAGGAGCAAACAGGGTCTAAGTCCA |
| | | C |
| 566 | 4660_Human_quadMut_14A_37A_46T_72A_n1 | GGGGGAGGCTGCTAGTGAATATTAACCAAGGTCACCACAGTTATCTGAGGAGCAAACAGGGGCTAAGTCCA |
| | | A |
| 567 | 4661_Human_quadMut_14T_37T_48G_52T_n1 | GGGGGAGGCTGCTTGTGAATATTAACCAAGGTCACCTCAGTTATCGGGGGATCAAACAGGGGCTAAGTCCA |
| | | C |
| 568 | 4663_Human_quadMut_14T_37A_48G_72G_n1 | GGGGGAGGCTGCTTGTGAATATTAACCAAGGTCACCACAGTTATCGGGGGAGCAAACAGGGGCTAAGTCCA |
| | | G |
| 569 | 4668_Human_quadMut_14T_45T_46T_52T_n1 | GGGGGAGGCTGCTTGTGAATATTAACCAAGGTCACCCCAGTTATTTGAGGATCAAACAGGGGCTAAGTCCA |
| | | C |
| 570 | 4669_Human_quadMut_14A_45G_46T_62A_n1 | GGGGGAGGCTGCTAGTGAATATTAACCAAGGTCACCCCAGTTATGTGAGGAGCAAACAGGGACTAAGTCCA |
| | | C |
| 571 | 4676_Human_quadMut_14A_45T_62A_72A_n1 | GGGGGAGGCTGCTAGTGAATATTAACCAAGGTCACCCCAGTTATTGGAGGAGCAAACAGGGACTAAGTCCA |
| | | A |
| 572 | 4678_Human_quadMut_14A_46T_48G_62C_n1 | GGGGGAGGCTGCTAGTGAATATTAACCAAGGTCACCCCAGTTATCTGGGGAGCAAACAGGGCCTAAGTCCA |
| | | C |
| 573 | 4682_Human_quadMut_14A_46A_62C_72A_n1 | GGGGGAGGCTGCTAGTGAATATTAACCAAGGTCACCCCAGTTATCAGAGGAGCAAACAGGGCCTAAGTCCA |
| | | A |
| 574 | 4683_Human_quadMut_14A_48G_52T_62A_n1 | GGGGGAGGCTGCTAGTGAATATTAACCAAGGTCACCCCAGTTATCGGGGGATCAAACAGGGACTAAGTCCA |
| | | C |
| 575 | 4692_Human_quadMut_35G_37T_46A_48G_n1 | GGGGGAGGCTGCTGGTGAATATTAACCAAGGTCAGCTCAGTTATCAGGGGAGCAAACAGGGGCTAAGTCCA |
| | | C |
| 576 | 4693_Human_quadMut_35A_37A_46T_52C_n1 | GGGGGAGGCTGCTGGTGAATATTAACCAAGGTCAACACAGTTATCTGAGGACCAAACAGGGGCTAAGTCCA |
| | | C |
| 577 | 4694_Human_quadMut_35T_37A_46T_62A_n1 | GGGGGAGGCTGCTGGTGAATATTAACCAAGGTCATCACAGTTATCTGAGGAGCAAACAGGGACTAAGTCCA |
| | | C |
| 578 | 4695_Human_quadMut_35A_37A_46A_72A_n1 | GGGGGAGGCTGCTGGTGAATATTAACCAAGGTCAACACAGTTATCAGAGGAGCAAACAGGGGCTAAGTCCA |
| | | A |
| 579 | 4696_Human_quadMut_35G_37T_48G_52T_n1 | GGGGGAGGCTGCTGGTGAATATTAACCAAGGTCAGCTCAGTTATCGGGGGATCAAACAGGGGCTAAGTCCA |
| | | C |
| 580 | 4697_Human_quadMut_35T_37A_48T_62T_n1 | GGGGGAGGCTGCTGGTGAATATTAACCAAGGTCATCACAGTTATCGGTGGAGCAAACAGGGTCTAAGTCCA |
| | | C |
| 581 | 4698_Human_quadMut_35A_37A_48G_72G_n1 | GGGGGAGGCTGCTGGTGAATATTAACCAAGGTCAACACAGTTATCGGGGGAGCAAACAGGGGCTAAGTCCA |
| | | G |
| 582 | 4699_Human_quadMut_35A_37A_52T_62C_n1 | GGGGGAGGCTGCTGGTGAATATTAACCAAGGTCAACACAGTTATCGGAGGATCAAACAGGGCCTAAGTCCA |
| | | C |
| 583 | 4700_Human_quadMut_35G_37T_52T_72A_n1 | GGGGGAGGCTGCTGGTGAATATTAACCAAGGTCAGCTCAGTTATCGGAGGATCAAACAGGGGCTAAGTCCA |
| | | A |
| 584 | 4702_Human_quadMut_35T_45T_46A_48G_n1 | GGGGGAGGCTGCTGGTGAATATTAACCAAGGTCATCCCAGTTATTAGGGGAGCAAACAGGGGCTAAGTCCA |
| | | C |
| 585 | 4704_Human_quadMut_35G_45T_46T_62C_n1 | GGGGGAGGCTGCTGGTGAATATTAACCAAGGTCAGCCCAGTTATTTGAGGAGCAAACAGGGCCTAAGTCCA |
| | | C |
| 586 | 4705_Human_quadMut_35T_45T_46T_72G_n1 | GGGGGAGGCTGCTGGTGAATATTAACCAAGGTCATCCCAGTTATTTGAGGAGCAAACAGGGGCTAAGTCCA |
| | | G |
| 587 | 4706_Human_quadMut_35A_45G_48T_52T_n1 | GGGGGAGGCTGCTGGTGAATATTAACCAAGGTCAACCCAGTTATGGGTGGATCAAACAGGGGCTAAGTCCA |
| | | C |
| 588 | 4709_Human_quadMut_35A_45A_52T_62T_n1 | GGGGGAGGCTGCTGGTGAATATTAACCAAGGTCAACCCAGTTATAGGAGGATCAAACAGGGTCTAAGTCCA |
| | | C |
| 589 | 4712_Human_quadMut_35T_46A_48G_52A_n1 | GGGGGAGGCTGCTGGTGAATATTAACCAAGGTCATCCCAGTTATCAGGGGAACAAACAGGGGCTAAGTCCA |
| | | C |
| 590 | 4714_Human_quadMut_35A_46T_48T_72T_n1 | GGGGGAGGCTGCTGGTGAATATTAACCAAGGTCAACCCAGTTATCTGTGGAGCAAACAGGGGCTAAGTCCA |
| | | T |
| 591 | 4717_Human_quadMut_35T_46A_62C_72A_n1 | GGGGGAGGCTGCTGGTGAATATTAACCAAGGTCATCCCAGTTATCAGAGGAGCAAACAGGGCCTAAGTCCA |
| | | A |
| 592 | 4718_Human_quadMut_35T_48G_52C_62C_n1 | GGGGGAGGCTGCTGGTGAATATTAACCAAGGTCATCCCAGTTATCGGGGGACCAAACAGGGCCTAAGTCCA |
| | | C |
| 593 | 4720_Human_quadMut_35G_48G_62C_72T_n1 | GGGGGAGGCTGCTGGTGAATATTAACCAAGGTCAGCCCAGTTATCGGGGGAGCAAACAGGGCCTAAGTCCA |
| | | T |
| 594 | 4723_Human_quadMut_37T_45G_46A_52A_n1 | GGGGGAGGCTGCTGGTGAATATTAACCAAGGTCACCTCAGTTATGAGAGGAACAAACAGGGGCTAAGTCCA |
| | | C |
| 595 | 4728_Human_quadMut_37T_45G_48T_72G_n1 | GGGGGAGGCTGCTGGTGAATATTAACCAAGGTCACCTCAGTTATGGGTGGAGCAAACAGGGGCTAAGTCCA |
| | | G |
| 596 | 4730_Human_quadMut_37T_45T_52A_72T_n1 | GGGGGAGGCTGCTGGTGAATATTAACCAAGGTCACCTCAGTTATTGGAGGAACAAACAGGGGCTAAGTCCA |
| | | T |
| 597 | 4733_Human_quadMut_37T_46T_48T_62T_n1 | GGGGGAGGCTGCTGGTGAATATTAACCAAGGTCACCTCAGTTATCTGTGGAGCAAACAGGGTCTAAGTCCA |
| | | C |
| 598 | 4734_Human_quadMut_37A_46T_48G_72G_n1 | GGGGGAGGCTGCTGGTGAATATTAACCAAGGTCACCACAGTTATCTGGGGAGCAAACAGGGGCTAAGTCCA |
| | | G |
| 599 | 4735_Human_quadMut_37A_46A_52T_62A_n1 | GGGGGAGGCTGCTGGTGAATATTAACCAAGGTCACCACAGTTATCAGAGGATCAAACAGGGACTAAGTCCA |
| | | C |
| 600 | 4736_Human_quadMut_37T_46T_52A_72A_n1 | GGGGGAGGCTGCTGGTGAATATTAACCAAGGTCACCTCAGTTATCTGAGGAACAAACAGGGGCTAAGTCCA |
| | | A |
| 601 | 4738_Human_quadMut_37T_48G_52A_62A_n1 | GGGGGAGGCTGCTGGTGAATATTAACCAAGGTCACCTCAGTTATCGGGGGAACAAACAGGGACTAAGTCCA |
| | | C |
| 602 | 4739_Human_quadMut_37A_48G_52A_72A_n1 | GGGGGAGGCTGCTGGTGAATATTAACCAAGGTCACCACAGTTATCGGGGGAACAAACAGGGGCTAAGTCCA |
| | | A |
| 603 | 4740_Human_quadMut_37T_48G_62C_72A_n1 | GGGGGAGGCTGCTGGTGAATATTAACCAAGGTCACCTCAGTTATCGGGGGAGCAAACAGGGCCTAAGTCCA |
| | | A |
| 604 | 4742_Human_quadMut_45T_46T_48G_52C_n1 | GGGGGAGGCTGCTGGTGAATATTAACCAAGGTCACCCCAGTTATTTGGGGACCAAACAGGGGCTAAGTCCA |
| | | C |
| 605 | 4751_Human_quadMut_45T_52T_62T_72G_n1 | GGGGGAGGCTGCTGGTGAATATTAACCAAGGTCACCCCAGTTATTGGAGGATCAAACAGGGTCTAAGTCCA |
| | | G |
| 606 | 4755_Human_quadMut_46T_52T_62T_72G_n1 | GGGGGAGGCTGCTGGTGAATATTAACCAAGGTCACCCCAGTTATCTGAGGATCAAACAGGGTCTAAGTCCA |
| | | G |
| 607 | 4761_Human_quadMut_1T_3T_8T_46A_n1 | TGTGGAGTCTGCTGGTGAATATTAACCAAGGTCACCCCAGTTATCAGAGGAGCAAACAGGGGCTAAGTCCA |
| | | C |
| 608 | 4763_Human_quadMut_1A_3T_8T_52T_n1 | AGTGGAGTCTGCTGGTGAATATTAACCAAGGTCACCCCAGTTATCGGAGGATCAAACAGGGGCTAAGTCCA |
| | | C |
| 609 | 4766_Human_quadMut_1A_3A_14A_35G_n1 | AGAGGAGGCTGCTAGTGAATATTAACCAAGGTCAGCCCAGTTATCGGAGGAGCAAACAGGGGCTAAGTCCA |
| | | C |
| 610 | 4770_Human_quadMut_1A_3A_14A_48T_n1 | AGAGGAGGCTGCTAGTGAATATTAACCAAGGTCACCCCAGTTATCGGTGGAGCAAACAGGGGCTAAGTCCA |
| | | C |
| 611 | 4771_Human_quadMut_1A_3T_14A_52T_n1 | AGTGGAGGCTGCTAGTGAATATTAACCAAGGTCACCCCAGTTATCGGAGGATCAAACAGGGGCTAAGTCCA |
| | | C |
| 612 | 4772_Human_quadMut_1T_3T_14A_62T_n1 | TGTGGAGGCTGCTAGTGAATATTAACCAAGGTCACCCCAGTTATCGGAGGAGCAAACAGGGTCTAAGTCCA |
| | | C |
| 613 | 4776_Human_quadMut_1A_3A_35A_48G_n1 | AGAGGAGGCTGCTGGTGAATATTAACCAAGGTCAACCCAGTTATCGGGGGAGCAAACAGGGGCTAAGTCCA |
| | | C |
| 614 | 4777_Human_quadMut_1A_3A_35T_52C_n1 | AGAGGAGGCTGCTGGTGAATATTAACCAAGGTCATCCCAGTTATCGGAGGACCAAACAGGGGCTAAGTCCA |
| | | C |
| 615 | 4778_Human_quadMut_1A_3T_35A_62T_n1 | AGTGGAGGCTGCTGGTGAATATTAACCAAGGTCAACCCAGTTATCGGAGGAGCAAACAGGGTCTAAGTCCA |
| | | C |
| 616 | 4779_Human_quadMut_1A_3A_35G_72A_n1 | AGAGGAGGCTGCTGGTGAATATTAACCAAGGTCAGCCCAGTTATCGGAGGAGCAAACAGGGGCTAAGTCCA |
| | | A |
| 617 | 4780_Human_quadMut_1T_3A_37T_45T_n1 | TGAGGAGGCTGCTGGTGAATATTAACCAAGGTCACCTCAGTTATTGGAGGAGCAAACAGGGGCTAAGTCCA |
| | | C |
| 618 | 4783_Human_quadMut_1T_3A_37T_52A_n1 | TGAGGAGGCTGCTGGTGAATATTAACCAAGGTCACCTCAGTTATCGGAGGAACAAACAGGGGCTAAGTCCA |
| | | C |
| 619 | 4784_Human_quadMut_1T_3A_37A_62T_n1 | TGAGGAGGCTGCTGGTGAATATTAACCAAGGTCACCACAGTTATCGGAGGAGCAAACAGGGTCTAAGTCCA |
| | | C |
| 620 | 4788_Human_quadMut_1T_3A_45A_52C_n1 | TGAGGAGGCTGCTGGTGAATATTAACCAAGGTCACCCCAGTTATAGGAGGACCAAACAGGGGCTAAGTCCA |
| | | C |
| 621 | 4790_Human_quadMut_1A_3T_45G_72A_n1 | AGTGGAGGCTGCTGGTGAATATTAACCAAGGTCACCCCAGTTATGGGAGGAGCAAACAGGGGCTAAGTCCA |
| | | A |
| 622 | 4791_Human_quadMut_1T_3T_46A_48T_n1 | TGTGGAGGCTGCTGGTGAATATTAACCAAGGTCACCCCAGTTATCAGTGGAGCAAACAGGGGCTAAGTCCA |
| | | C |
| 623 | 4792_Human_quadMut_1A_3A_46T_52A_n1 | AGAGGAGGCTGCTGGTGAATATTAACCAAGGTCACCCCAGTTATCTGAGGAACAAACAGGGGCTAAGTCCA |
| | | C |
| 624 | 4796_Human_quadMut_1T_3A_48T_62T_n1 | TGAGGAGGCTGCTGGTGAATATTAACCAAGGTCACCCCAGTTATCGGTGGAGCAAACAGGGTCTAAGTCCA |
| | | C |
| 625 | 4798_Human_quadMut_1T_3A_52A_62C_n1 | TGAGGAGGCTGCTGGTGAATATTAACCAAGGTCACCCCAGTTATCGGAGGAACAAACAGGGCCTAAGTCCA |
| | | C |
| 626 | 4805_Human_quadMut_1T_8A_14T_48T_n1 | TGGGGAGACTGCTTGTGAATATTAACCAAGGTCACCCCAGTTATCGGTGGAGCAAACAGGGGCTAAGTCCA |
| | | C |
| 627 | 4806_Human_quadMut_1A_8A_14A_52T_n1 | AGGGGAGACTGCTAGTGAATATTAACCAAGGTCACCCCAGTTATCGGAGGATCAAACAGGGGCTAAGTCCA |
| | | C |
| 728 | 4809_Human_quadMut_1A_8C_35A_37A_n1 | AGGGGAGCCTGCTGGTGAATATTAACCAAGGTCAACACAGTTATCGGAGGAGCAAACAGGGGCTAAGTCCA |
| | | C |
| 629 | 4810_Human_quadMut_1A_8C_35A_45A_n1 | AGGGGAGCCTGCTGGTGAATATTAACCAAGGTCAACCCAGTTATAGGAGGAGCAAACAGGGGCTAAGTCCA |
| | | C |
| 630 | 4811_Human_quadMut_1A_8T_35G_46A_n1 | AGGGGAGTCTGCTGGTGAATATTAACCAAGGTCAGCCCAGTTATCAGAGGAGCAAACAGGGGCTAAGTCCA |
| | | C |
| 631 | 4812_Human_quadMut_1T_8A_35T_48T_n1 | TGGGGAGACTGCTGGTGAATATTAACCAAGGTCATCCCAGTTATCGGTGGAGCAAACAGGGGCTAAGTCCA |
| | | C |
| 632 | 4819_Human_quadMut_1A_8T_37T_52T_n1 | AGGGGAGTCTGCTGGTGAATATTAACCAAGGTCACCTCAGTTATCGGAGGATCAAACAGGGGCTAAGTCCA |
| | | C |
| 633 | 4820_Human_quadMut_1A_8A_37T_72G_n1 | AGGGGAGACTGCTGGTGAATATTAACCAAGGTCACCTCAGTTATCGGAGGAGCAAACAGGGGCTAAGTCCA |
| | | G |
| 634 | 4822_Human_quadMut_1T_8A_45A_48G_n1 | TGGGGAGACTGCTGGTGAATATTAACCAAGGTCACCCCAGTTATAGGGGGAGCAAACAGGGGCTAAGTCCA |
| | | C |
| 635 | 4826_Human_quadMut_1T_8T_46T_48T_n1 | TGGGGAGTCTGCTGGTGAATATTAACCAAGGTCACCCCAGTTATCTGTGGAGCAAACAGGGGCTAAGTCCA |
| | | C |
| 636 | 4833_Human_quadMut_1T_8C_52T_72G_n1 | TGGGGAGCCTGCTGGTGAATATTAACCAAGGTCACCCCAGTTATCGGAGGATCAAACAGGGGCTAAGTCCA |
| | | G |
| 637 | 4839_Human_quadMut_1A_14T_35T_52T_n1 | AGGGGAGGCTGCTTGTGAATATTAACCAAGGTCATCCCAGTTATCGGAGGATCAAACAGGGGCTAAGTCCA |
| | | C |
| 638 | 4853_Human_quadMut_1A_14A_46A_48G_n1 | AGGGGAGGCTGCTAGTGAATATTAACCAAGGTCACCCCAGTTATCAGGGGAGCAAACAGGGGCTAAGTCCA |
| | | C |
| 639 | 4864_Human_quadMut_1A_35A_37A_46T_n1 | AGGGGAGGCTGCTGGTGAATATTAACCAAGGTCAACACAGTTATCTGAGGAGCAAACAGGGGCTAAGTCCA |
| | | C |
| 640 | 4867_Human_quadMut_1A_35A_37T_62C_n1 | AGGGGAGGCTGCTGGTGAATATTAACCAAGGTCAACTCAGTTATCGGAGGAGCAAACAGGGCCTAAGTCCA |
| | | C |
| 641 | 4872_Human_quadMut_1T_35A_45T_62T_n1 | TGGGGAGGCTGCTGGTGAATATTAACCAAGGTCAACCCAGTTATTGGAGGAGCAAACAGGGTCTAAGTCCA |
| | | C |
| 642 | 4876_Human_quadMut_1A_35A_46A_62C_n1 | AGGGGAGGCTGCTGGTGAATATTAACCAAGGTCAACCCAGTTATCAGAGGAGCAAACAGGGCCTAAGTCCA |
| | | C |
| 643 | 4879_Human_quadMut_1A_35A_48T_62T_n1 | AGGGGAGGCTGCTGGTGAATATTAACCAAGGTCAACCCAGTTATCGGTGGAGCAAACAGGGTCTAAGTCCA |
| | | C |
| 644 | 4880_Human_quadMut_1A_35G_48G_72T_n1 | AGGGGAGGCTGCTGGTGAATATTAACCAAGGTCAGCCCAGTTATCGGGGGAGCAAACAGGGGCTAAGTCCA |
| | | T |
| 645 | 4884_Human_quadMut_1A_37T_45T_46T_n1 | AGGGGAGGCTGCTGGTGAATATTAACCAAGGTCACCTCAGTTATTTGAGGAGCAAACAGGGGCTAAGTCCA |
| | | C |
| 646 | 4887_Human_quadMut_1A_37A_45T_62C_n1 | AGGGGAGGCTGCTGGTGAATATTAACCAAGGTCACCACAGTTATTGGAGGAGCAAACAGGGCCTAAGTCCA |
| | | C |
| 647 | 4890_Human_quadMut_1T_37A_46T_52T_n1 | TGGGGAGGCTGCTGGTGAATATTAACCAAGGTCACCACAGTTATCTGAGGATCAAACAGGGGCTAAGTCCA |
| | | C |
| 648 | 4891_Human_quadMut_1A_37A_46T_62A_n1 | AGGGGAGGCTGCTGGTGAATATTAACCAAGGTCACCACAGTTATCTGAGGAGCAAACAGGGACTAAGTCCA |
| | | C |
| 649 | 4892_Human_quadMut_1T_37T_46A_72A_n1 | TGGGGAGGCTGCTGGTGAATATTAACCAAGGTCACCTCAGTTATCAGAGGAGCAAACAGGGGCTAAGTCCA |
| | | A |
| 650 | 4894_Human_quadMut_1A_37A_48G_62A_n1 | AGGGGAGGCTGCTGGTGAATATTAACCAAGGTCACCACAGTTATCGGGGGAGCAAACAGGGACTAAGTCCA |
| | | C |
| 651 | 4896_Human_quadMut_1T_37A_52T_62A_n1 | TGGGGAGGCTGCTGGTGAATATTAACCAAGGTCACCACAGTTATCGGAGGATCAAACAGGGACTAAGTCCA |
| | | C |
| 652 | 4898_Human_quadMut_1A_37T_62T_72T_n1 | AGGGGAGGCTGCTGGTGAATATTAACCAAGGTCACCTCAGTTATCGGAGGAGCAAACAGGGTCTAAGTCCA |
| | | T |
| 653 | 4907_Human_quadMut_1A_45G_52A_72A_n1 | AGGGGAGGCTGCTGGTGAATATTAACCAAGGTCACCCCAGTTATGGGAGGAACAAACAGGGGCTAAGTCCA |
| | | A |
| 654 | 4911_Human_quadMut_1A_46A_52A_62T_n1 | AGGGGAGGCTGCTGGTGAATATTAACCAAGGTCACCCCAGTTATCAGAGGAACAAACAGGGTCTAAGTCCA |
| | | C |
| 655 | 4914_Human_quadMut_1A_48T_52T_62C_n1 | AGGGGAGGCTGCTGGTGAATATTAACCAAGGTCACCCCAGTTATCGGTGGATCAAACAGGGCCTAAGTCCA |
| | | C |
| 656 | 4923_Human_quadMut_3A_8A_14A_52A_n1 | GGAGGAGACTGCTAGTGAATATTAACCAAGGTCACCCCAGTTATCGGAGGAACAAACAGGGGCTAAGTCCA |
| | | C |
| 657 | 4926_Human_quadMut_3A_8C_35A_37A_n1 | GGAGGAGCCTGCTGGTGAATATTAACCAAGGTCAACACAGTTATCGGAGGAGCAAACAGGGGCTAAGTCCA |
| | | C |
| 658 | 4932_Human_quadMut_3A_8C_35A_72A_n1 | GGAGGAGCCTGCTGGTGAATATTAACCAAGGTCAACCCAGTTATCGGAGGAGCAAACAGGGGCTAAGTCCA |
| | | A |
| 659 | 4934_Human_quadMut_3A_8C_37T_46T_n1 | GGAGGAGCCTGCTGGTGAATATTAACCAAGGTCACCTCAGTTATCTGAGGAGCAAACAGGGGCTAAGTCCA |
| | | C |
| 660 | 4935_Human_quadMut_3T_8C_37A_48T_n1 | GGTGGAGCCTGCTGGTGAATATTAACCAAGGTCACCACAGTTATCGGTGGAGCAAACAGGGGCTAAGTCCA |
| | | C |
| 661 | 4943_Human_quadMut_3A_8A_46T_48T_n1 | GGAGGAGACTGCTGGTGAATATTAACCAAGGTCACCCCAGTTATCTGTGGAGCAAACAGGGGCTAAGTCCA |
| | | C |
| 662 | 4944_Human_quadMut_3A_8A_46A_52T_n1 | GGAGGAGACTGCTGGTGAATATTAACCAAGGTCACCCCAGTTATCAGAGGATCAAACAGGGGCTAAGTCCA |
| | | C |
| 663 | 4947_Human_quadMut_3T_8T_48T_52T_n1 | GGTGGAGTCTGCTGGTGAATATTAACCAAGGTCACCCCAGTTATCGGTGGATCAAACAGGGGCTAAGTCCA |
| | | C |
| 664 | 4951_Human_quadMut_3T_8A_52A_72G_n1 | GGTGGAGACTGCTGGTGAATATTAACCAAGGTCACCCCAGTTATCGGAGGAACAAACAGGGGCTAAGTCCA |
| | | G |
| 665 | 4952_Human_quadMut_3T_8C_62T_72A_n1 | GGTGGAGCCTGCTGGTGAATATTAACCAAGGTCACCCCAGTTATCGGAGGAGCAAACAGGGTCTAAGTCCA |
| | | A |
| 666 | 4955_Human_quadMut_3T_14T_35T_46A_n1 | GGTGGAGGCTGCTTGTGAATATTAACCAAGGTCATCCCAGTTATCAGAGGAGCAAACAGGGGCTAAGTCCA |
| | | C |
| 667 | 4956_Human_quadMut_3A_14A_35G_48G_n1 | GGAGGAGGCTGCTAGTGAATATTAACCAAGGTCAGCCCAGTTATCGGGGGAGCAAACAGGGGCTAAGTCCA |
| | | C |
| 668 | 4957_Human_quadMut_3T_14T_35T_52T_n1 | GGTGGAGGCTGCTTGTGAATATTAACCAAGGTCATCCCAGTTATCGGAGGATCAAACAGGGGCTAAGTCCA |
| | | C |
| 669 | 4975_Human_quadMut_3A_14A_48G_62T_n1 | GGAGGAGGCTGCTAGTGAATATTAACCAAGGTCACCCCAGTTATCGGGGGAGCAAACAGGGTCTAAGTCCA |
| | | C |
| 670 | 4976_Human_quadMut_3A_14A_48T_72A_n1 | GGAGGAGGCTGCTAGTGAATATTAACCAAGGTCACCCCAGTTATCGGTGGAGCAAACAGGGGCTAAGTCCA |
| | | A |
| 671 | 4977_Human_quadMut_3T_14A_52A_62T_n1 | GGTGGAGGCTGCTAGTGAATATTAACCAAGGTCACCCCAGTTATCGGAGGAACAAACAGGGTCTAAGTCCA |
| | | C |
| 672 | 4980_Human_quadMut_3T_35A_37T_46T_n1 | GGTGGAGGCTGCTGGTGAATATTAACCAAGGTCAACTCAGTTATCTGAGGAGCAAACAGGGGCTAAGTCCA |
| | | C |
| 673 | 4986_Human_quadMut_3T_35A_45A_48T_n1 | GGTGGAGGCTGCTGGTGAATATTAACCAAGGTCAACCCAGTTATAGGTGGAGCAAACAGGGGCTAAGTCCA |
| | | C |
| 674 | 4988_Human_quadMut_3T_35G_45T_62T_n1 | GGTGGAGGCTGCTGGTGAATATTAACCAAGGTCAGCCCAGTTATTGGAGGAGCAAACAGGGTCTAAGTCCA |
| | | C |
| 675 | 4991_Human_quadMut_3A_35A_46A_52C_n1 | GGAGGAGGCTGCTGGTGAATATTAACCAAGGTCAACCCAGTTATCAGAGGACCAAACAGGGGCTAAGTCCA |
| | | C |
| 676 | 4992_Human_quadMut_3A_35T_46A_62A_n1 | GGAGGAGGCTGCTGGTGAATATTAACCAAGGTCATCCCAGTTATCAGAGGAGCAAACAGGGACTAAGTCCA |
| | | C |
| 677 | 4993_Human_quadMut_3T_35T_46T_72T_n1 | GGTGGAGGCTGCTGGTGAATATTAACCAAGGTCATCCCAGTTATCTGAGGAGCAAACAGGGGCTAAGTCCA |
| | | T |
| 678 | 4995_Human_quadMut_3T_35A_48T_62A_n1 | GGTGGAGGCTGCTGGTGAATATTAACCAAGGTCAACCCAGTTATCGGTGGAGCAAACAGGGACTAAGTCCA |
| | | C |
| 679 | 4997_Human_quadMut_3A_35A_52T_62T_n1 | GGAGGAGGCTGCTGGTGAATATTAACCAAGGTCAACCCAGTTATCGGAGGATCAAACAGGGTCTAAGTCCA |
| | | C |
| 680 | 5000_Human_quadMut_3T_37T_45A_46A_n1 | GGTGGAGGCTGCTGGTGAATATTAACCAAGGTCACCTCAGTTATAAGAGGAGCAAACAGGGGCTAAGTCCA |
| | | C |
| 681 | 5003_Human_quadMut_3T_37A_45T_62T_n1 | GGTGGAGGCTGCTGGTGAATATTAACCAAGGTCACCACAGTTATTGGAGGAGCAAACAGGGTCTAAGTCCA |
| | | C |
| 682 | 5004_Human_quadMut_3A_37T_46A_48G_n1 | GGAGGAGGCTGCTGGTGAATATTAACCAAGGTCACCTCAGTTATCAGGGGAGCAAACAGGGGCTAAGTCCA |
| | | C |
| 683 | 5005_Human_quadMut_3A_37A_46T_52T_n1 | GGAGGAGGCTGCTGGTGAATATTAACCAAGGTCACCACAGTTATCTGAGGATCAAACAGGGGCTAAGTCCA |
| | | C |
| 684 | 5006_Human_quadMut_3A_37A_46A_62T_n1 | GGAGGAGGCTGCTGGTGAATATTAACCAAGGTCACCACAGTTATCAGAGGAGCAAACAGGGTCTAAGTCCA |
| | | C |
| 685 | 5008_Human_quadMut_3A_37A_48G_52T_n1 | GGAGGAGGCTGCTGGTGAATATTAACCAAGGTCACCACAGTTATCGGGGGATCAAACAGGGGCTAAGTCCA |
| | | C |
| 686 | 5010_Human_quadMut_3T_37A_48G_72A_n1 | GGTGGAGGCTGCTGGTGAATATTAACCAAGGTCACCACAGTTATCGGGGGAGCAAACAGGGGCTAAGTCCA |
| | | A |
| 687 | 5019_Human_quadMut_3T_45G_48G_62T_n1 | GGTGGAGGCTGCTGGTGAATATTAACCAAGGTCACCCCAGTTATGGGGGGAGCAAACAGGGTCTAAGTCCA |
| | | C |
| 688 | 5025_Human_quadMut_3A_46A_48T_62C_n1 | GGAGGAGGCTGCTGGTGAATATTAACCAAGGTCACCCCAGTTATCAGTGGAGCAAACAGGGCCTAAGTCCA |
| | | C |
| 689 | 5027_Human_quadMut_3T_46A_52A_62T_n1 | GGTGGAGGCTGCTGGTGAATATTAACCAAGGTCACCCCAGTTATCAGAGGAACAAACAGGGTCTAAGTCCA |
| | | C |
| 690 | 5030_Human_quadMut_3T_48G_52T_62T_n1 | GGTGGAGGCTGCTGGTGAATATTAACCAAGGTCACCCCAGTTATCGGGGGATCAAACAGGGTCTAAGTCCA |
| | | C |
| 691 | 5032_Human_quadMut_3A_48T_62C_72A_n1 | GGAGGAGGCTGCTGGTGAATATTAACCAAGGTCACCCCAGTTATCGGTGGAGCAAACAGGGCCTAAGTCCA |
| | | A |
| 692 | 5033_Human_quadMut_3T_52T_62C_72G_n1 | GGTGGAGGCTGCTGGTGAATATTAACCAAGGTCACCCCAGTTATCGGAGGATCAAACAGGGCCTAAGTCCA |
| | | G |
| 693 | 5036_Human_quadMut_8T_14A_35T_46T_n1 | GGGGGAGTCTGCTAGTGAATATTAACCAAGGTCATCCCAGTTATCTGAGGAGCAAACAGGGGCTAAGTCCA |
| | | C |
| 694 | 5037_Human_quadMut_8C_14T_35T_48G_n1 | GGGGGAGCCTGCTTGTGAATATTAACCAAGGTCATCCCAGTTATCGGGGGAGCAAACAGGGGCTAAGTCCA |
| | | C |
| 695 | 5040_Human_quadMut_8T_14A_35G_72G_n1 | GGGGGAGTCTGCTAGTGAATATTAACCAAGGTCAGCCCAGTTATCGGAGGAGCAAACAGGGGCTAAGTCCA |
| | | G |
| 696 | 5041_Human_quadMut_8T_14A_37T_45T_n1 | GGGGGAGTCTGCTAGTGAATATTAACCAAGGTCACCTCAGTTATTGGAGGAGCAAACAGGGGCTAAGTCCA |
| | | C |
| 697 | 5042_Human_quadMut_8C_14A_37T_46A_n1 | GGGGGAGCCTGCTAGTGAATATTAACCAAGGTCACCTCAGTTATCAGAGGAGCAAACAGGGGCTAAGTCCA |
| | | C |
| 698 | 5043_Human_quadMut_8A_14A_37A_48G_n1 | GGGGGAGACTGCTAGTGAATATTAACCAAGGTCACCACAGTTATCGGGGGAGCAAACAGGGGCTAAGTCCA |
| | | C |
| 699 | 5051_Human_quadMut_8C_14T_46A_52T_n1 | GGGGGAGCCTGCTTGTGAATATTAACCAAGGTCACCCCAGTTATCAGAGGATCAAACAGGGGCTAAGTCCA |
| | | C |
| 700 | 5052_Human_quadMut_8A_14T_46A_62C_n1 | GGGGGAGACTGCTTGTGAATATTAACCAAGGTCACCCCAGTTATCAGAGGAGCAAACAGGGCCTAAGTCCA |
| | | C |
| 701 | 5056_Human_quadMut_8C_14A_48G_72A_n1 | GGGGGAGCCTGCTAGTGAATATTAACCAAGGTCACCCCAGTTATCGGGGGAGCAAACAGGGGCTAAGTCCA |
| | | A |
| 702 | 5057_Human_quadMut_8T_14A_52A_62C_n1 | GGGGGAGTCTGCTAGTGAATATTAACCAAGGTCACCCCAGTTATCGGAGGAACAAACAGGGCCTAAGTCCA |
| | | C |
| 703 | 5063_Human_quadMut_8A_35A_37A_52T_n1 | GGGGGAGACTGCTGGTGAATATTAACCAAGGTCAACACAGTTATCGGAGGATCAAACAGGGGCTAAGTCCA |
| | | C |
| 704 | 5071_Human_quadMut_8T_35A_46T_48T_n1 | GGGGGAGTCTGCTGGTGAATATTAACCAAGGTCAACCCAGTTATCTGTGGAGCAAACAGGGGCTAAGTCCA |
| | | C |
| 705 | 5072_Human_quadMut_8A_35G_46T_52T_n1 | GGGGGAGACTGCTGGTGAATATTAACCAAGGTCAGCCCAGTTATCTGAGGATCAAACAGGGGCTAAGTCCA |
| | | C |
| 706 | 5073_Human_quadMut_8T_35G_46T_62T_n1 | GGGGGAGTCTGCTGGTGAATATTAACCAAGGTCAGCCCAGTTATCTGAGGAGCAAACAGGGTCTAAGTCCA |
| | | C |
| 707 | 5074_Human_quadMut_8T_35G_46T_72A_n1 | GGGGGAGTCTGCTGGTGAATATTAACCAAGGTCAGCCCAGTTATCTGAGGAGCAAACAGGGGCTAAGTCCA |
| | | A |
| 708 | 5075_Human_quadMut_8T_35T_48G_52T_n1 | GGGGGAGTCTGCTGGTGAATATTAACCAAGGTCATCCCAGTTATCGGGGGATCAAACAGGGGCTAAGTCCA |
| | | C |
| 709 | 5080_Human_quadMut_8A_35T_62T_72G_n1 | GGGGGAGACTGCTGGTGAATATTAACCAAGGTCATCCCAGTTATCGGAGGAGCAAACAGGGTCTAAGTCCA |
| | | G |
| 710 | 5081_Human_quadMut_8A_37A_45A_46T_n1 | GGGGGAGACTGCTGGTGAATATTAACCAAGGTCACCACAGTTATATGAGGAGCAAACAGGGGCTAAGTCCA |
| | | C |
| 711 | 5083_Human_quadMut_8T_37T_45T_52T_n1 | GGGGGAGTCTGCTGGTGAATATTAACCAAGGTCACCTCAGTTATTGGAGGATCAAACAGGGGCTAAGTCCA |
| | | C |
| 712 | 5085_Human_quadMut_8T_37T_45T_72T_n1 | GGGGGAGTCTGCTGGTGAATATTAACCAAGGTCACCTCAGTTATTGGAGGAGCAAACAGGGGCTAAGTCCA |
| | | T |
| 713 | 5086_Human_quadMut_8A_37T_46T_48G_n1 | GGGGGAGACTGCTGGTGAATATTAACCAAGGTCACCTCAGTTATCTGGGGAGCAAACAGGGGCTAAGTCCA |
| | | C |
| 714 | 5087_Human_quadMut_8A_37A_46A_52A_n1 | GGGGGAGACTGCTGGTGAATATTAACCAAGGTCACCACAGTTATCAGAGGAACAAACAGGGGCTAAGTCCA |
| | | C |
| 715 | 5088_Human_quadMut_8A_37A_46A_62C_n1 | GGGGGAGACTGCTGGTGAATATTAACCAAGGTCACCACAGTTATCAGAGGAGCAAACAGGGCCTAAGTCCA |
| | | C |
| 716 | 5089_Human_quadMut_8T_37A_46T_72A_n1 | GGGGGAGTCTGCTGGTGAATATTAACCAAGGTCACCACAGTTATCTGAGGAGCAAACAGGGGCTAAGTCCA |
| | | A |
| 717 | 5090_Human_quadMut_8A_37A_48G_52T_n1 | GGGGGAGACTGCTGGTGAATATTAACCAAGGTCACCACAGTTATCGGGGGATCAAACAGGGGCTAAGTCCA |
| | | C |
| 718 | 5092_Human_quadMut_8T_37T_48T_72G_n1 | GGGGGAGTCTGCTGGTGAATATTAACCAAGGTCACCTCAGTTATCGGTGGAGCAAACAGGGGCTAAGTCCA |
| | | G |
| 719 | 5093_Human_quadMut_8C_37A_52T_62T_n1 | GGGGGAGCCTGCTGGTGAATATTAACCAAGGTCACCACAGTTATCGGAGGATCAAACAGGGTCTAAGTCCA |
| | | C |
| 720 | 5100_Human_quadMut_8T_45G_48G_52A_n1 | GGGGGAGTCTGCTGGTGAATATTAACCAAGGTCACCCCAGTTATGGGGGGAACAAACAGGGGCTAAGTCCA |
| | | C |
| 721 | 5104_Human_quadMut_8T_45G_52T_72G_n1 | GGGGGAGTCTGCTGGTGAATATTAACCAAGGICACCCCAGTTATGGGAGGATCAAACAGGGGCTAAGTCCA |
| | | G |
| 722 | 5107_Human_quadMut_8A_46A_48T_62T_n1 | GGGGGAGACTGCTGGTGAATATTAACCAAGGTCACCCCAGTTATCAGTGGAGCAAACAGGGTCTAAGTCCA |
| | | C |
| 723 | 5109_Human_quadMut_8A_46T_52T_62T_n1 | GGGGGAGACTGCTGGTGAATATTAACCAAGGTCACCCCAGTTATCTGAGGATCAAACAGGGTCTAAGTCCA |
| | | C |
| 724 | 5110_Human_quadMut_8C_46T_52C_72A_n1 | GGGGGAGCCTGCTGGTGAATATTAACCAAGGTCACCCCAGTTATCTGAGGACCAAACAGGGGCTAAGTCCA |
| | | A |
| 725 | 5115_Human_quadMut_8T_52A_62A_72T_n1 | GGGGGAGTCTGCTGGTGAATATTAACCAAGGTCACCCCAGTTATCGGAGGAACAAACAGGGACTAAGTCCA |
| | | T |
| 726 | 5119_Human_quadMut_14A_35G_37A_52A_n1 | GGGGGAGGCTGCTAGTGAATATTAACCAAGGTCAGCACAGTTATCGGAGGAACAAACAGGGGCTAAGTCCA |
| | | C |
| 727 | 5128_Human_quadMut_14T_35T_46A_52A_n1 | GGGGGAGGCTGCTTGTGAATATTAACCAAGGTCATCCCAGTTATCAGAGGAACAAACAGGGGCTAAGTCCA |
| | | C |
| 728 | 5132_Human_quadMut_14A_35G_48T_62T_n1 | GGGGGAGGCTGCTAGTGAATATTAACCAAGGTCAGCCCAGTTATCGGTGGAGCAAACAGGGTCTAAGTCCA |
| | | C |
| 729 | 5133_Human_quadMut_14A_35T_48T_72G_n1 | GGGGGAGGCTGCTAGTGAATATTAACCAAGGTCATCCCAGTTATCGGTGGAGCAAACAGGGGCTAAGTCCA |
| | | G |
| 730 | 5134_Human_quadMut_14T_35G_52T_62C_n1 | GGGGGAGGCTGCTTGTGAATATTAACCAAGGTCAGCCCAGTTATCGGAGGATCAAACAGGGCCTAAGTCCA |
| | | C |
| 731 | 5135_Human_quadMut_14T_35A_52T_72G_n1 | GGGGGAGGCTGCTTGTGAATATTAACCAAGGTCAACCCAGTTATCGGAGGATCAAACAGGGGCTAAGTCCA |
| | | G |
| 732 | 5140_Human_quadMut_14A_37T_45G_62T_n1 | GGGGGAGGCTGCTAGTGAATATTAACCAAGGTCACCTCAGTTATGGGAGGAGCAAACAGGGTCTAAGTCCA |
| | | C |
| 733 | 5142_Human_quadMut_14A_37A_46A_48T_n1 | GGGGGAGGCTGCTAGTGAATATTAACCAAGGTCACCACAGTTATCAGTGGAGCAAACAGGGGCTAAGTCCA |
| | | C |
| 734 | 5145_Human_quadMut_14A_37T_46T_72A_n1 | GGGGGAGGCTGCTAGTGAATATTAACCAAGGTCACCTCAGTTATCTGAGGAGCAAACAGGGGCTAAGTCCA |
| | | A |
| 735 | 5146_Human_quadMut_14A_37T_48G_52A_n | GGGGGAGGCTGCTAGTGAATATTAACCAAGGTCACCTCAGTTATCGGGGGAACAAACAGGGGCTAAGTCCA |
| | | C |
| 736 | 5147_Human_quadMut_14A_37T_48T_62C_n1 | GGGGGAGGCTGCTAGTGAATATTAACCAAGGTCACCTCAGTTATCGGTGGAGCAAACAGGGCCTAAGTCCA |
| | | C |
| 737 | 5148_Human_quadMut_14A_37T_48G_72A_n1 | GGGGGAGGCTGCTAGTGAATATTAACCAAGGTCACCTCAGTTATCGGGGGAGCAAACAGGGGCTAAGTCCA |
| | | A |
| 738 | 5155_Human_quadMut_14T_45T_46T_72G_n1 | GGGGGAGGCTGCTTGTGAATATTAACCAAGGTCACCCCAGTTATTTGAGGAGCAAACAGGGGCTAAGTCCA |
| | | G |
| 739 | 5157_Human_quadMut_14T_45G_48G_62T_n1 | GGGGGAGGCTGCTTGTGAATATTAACCAAGGTCACCCCAGTTATGGGGGGAGCAAACAGGGTCTAAGTCCA |
| | | C |
| 740 | 5164_Human_quadMut_14T_46A_52T_62T_n1 | GGGGGAGGCTGCTTGTGAATATTAACCAAGGTCACCCCAGTTATCAGAGGATCAAACAGGGTCTAAGTCCA |
| | | C |
| 741 | 5165_Human_quadMut_14T_46A_52T_72G_n1 | GGGGGAGGCTGCTTGTGAATATTAACCAAGGTCACCCCAGTTATCAGAGGATCAAACAGGGGCTAAGTCCA |
| | | G |
| 742 | 5167_Human_quadMut_14A_48G_52A_62C_n1 | GGGGGAGGCTGCTAGTGAATATTAACCAAGGTCACCCCAGTTATCGGGGGAACAAACAGGGCCTAAGTCCA |
| | | C |
| 743 | 5171_Human_quadMut_35G_37A_45T_46T_n1 | GGGGGAGGCTGCTGGTGAATATTAACCAAGGTCAGCACAGTTATTTGAGGAGCAAACAGGGGCTAAGTCCA |
| | | C |
| 744 | 5175_Human_quadMut_35T_37A_46A_48G_n1 | GGGGGAGGCTGCTGGTGAATATTAACCAAGGTCATCACAGTTATCAGGGGAGCAAACAGGGGCTAAGTCCA |
| | | C |
| 745 | 5177_Human_quadMut_35G_37A_46A_72A_n1 | GGGGGAGGCTGCTGGTGAATATTAACCAAGGTCAGCACAGTTATCAGAGGAGCAAACAGGGGCTAAGTCCA |
| | | A |
| 746 | 5178_Human_quadMut_35G_37A_48G_52A_n1 | GGGGGAGGCTGCTGGTGAATATTAACCAAGGTCAGCACAGTTATCGGGGGAACAAACAGGGGCTAAGTCCA |
| | | C |
| 747 | 5179_Human_quadMut_35T_37T_48T_62A_n1 | GGGGGAGGCTGCTGGTGAATATTAACCAAGGTCATCTCAGTTATCGGTGGAGCAAACAGGGACTAAGTCCA |
| | | C |
| 748 | 5181_Human_quadMut_35T_37A_52A_62A_n1 | GGGGGAGGCTGCTGGTGAATATTAACCAAGGTCATCACAGTTATCGGAGGAACAAACAGGGACTAAGTCCA |
| | | C |
| 749 | 5185_Human_quadMut_35T_45A_46T_52A_n1 | GGGGGAGGCTGCTGGTGAATATTAACCAAGGTCATCCCAGTTATATGAGGAACAAACAGGGGCTAAGTCCA |
| | | C |
| 750 | 5186_Human_quadMut_35T_45T_46T_62T_n1 | GGGGGAGGCTGCTGGTGAATATTAACCAAGGTCATCCCAGTTATTTGAGGAGCAAACAGGGTCTAAGTCCA |
| | | C |
| 751 | 5188_Human_quadMut_35A_45A_48T_52A_n1 | GGGGGAGGCTGCTGGTGAATATTAACCAAGGTCAACCCAGTTATAGGTGGAACAAACAGGGGCTAAGTCCA |
| | | C |
| 752 | 5194_Human_quadMut_35G_46T_48G_52C_n1 | GGGGGAGGCTGCTGGTGAATATTAACCAAGGTCAGCCCAGTTATCTGGGGACCAAACAGGGGCTAAGTCCA |
| | | C |
| 753 | 5195_Human_quadMut_35T_46A_48G_62A_n1 | GGGGGAGGCTGCTGGTGAATATTAACCAAGGTCATCCCAGTTATCAGGGGAGCAAACAGGGACTAAGTCCA |
| | | C |
| 754 | 5196_Human_quadMut_35A_46T_48G_72A_n1 | GGGGGAGGCTGCTGGTGAATATTAACCAAGGTCAACCCAGTTATCTGGGGAGCAAACAGGGGCTAAGTCCA |
| | | A |
| 755 | 5197_Human_quadMut_35A_46T_52C_62T_n1 | GGGGGAGGCTGCTGGTGAATATTAACCAAGGTCAACCCAGTTATCTGAGGACCAAACAGGGTCTAAGTCCA |
| | | C |
| 756 | 5200_Human_quadMut_35A_48G_52A_62A_n1 | GGGGGAGGCTGCTGGTGAATATTAACCAAGGTCAACCCAGTTATCGGGGGAACAAACAGGGACTAAGTCCA |
| | | C |
| 757 | 5201_Human_quadMut_35A_48T_52T_72A_n1 | GGGGGAGGCTGCTGGTGAATATTAACCAAGGTCAACCCAGTTATCGGTGGATCAAACAGGGGCTAAGTCCA |
| | | A |
| 758 | 5202_Human_quadMut_35T_48G_62C_72T_n1 | GGGGGAGGCTGCTGGTGAATATTAACCAAGGTCATCCCAGTTATCGGGGGAGCAAACAGGGCCTAAGTCCA |
| | | T |
| 759 | 5203_Human_quadMut_35T_52T_62T_72T_n1 | GGGGGAGGCTGCTGGTGAATATTAACCAAGGTCATCCCAGTTATCGGAGGATCAAACAGGGTCTAAGTCCA |
| | | T |
| 760 | 5204_Human_quadMut_37A_45G_46A_48G_n1 | GGGGGAGGCTGCTGGTGAATATTAACCAAGGTCACCACAGTTATGAGGGGAGCAAACAGGGGCTAAGTCCA |
| | | C |
| 761 | 5205_Human_quadMut_37A_45A_46A_62T_n1 | GGGGGAGGCTGCTGGTGAATATTAACCAAGGTCACCACAGTTATAAGAGGAGCAAACAGGGTCTAAGTCCA |
| | | C |
| 762 | 5208_Human_quadMut_37T_45T_48G_62T_n1 | GGGGGAGGCTGCTGGTGAATATTAACCAAGGTCACCTCAGTTATTGGGGGAGCAAACAGGGTCTAAGTCCA |
| | | C |
| 763 | 5210_Human_quadMut_37A_45G_52A_62C_n1 | GGGGGAGGCTGCTGGTGAATATTAACCAAGGTCACCACAGTTATGGGAGGAACAAACAGGGCCTAAGTCCA |
| | | C |
| 764 | 5213_Human_quadMut_37A_46A_48G_52T_n1 | GGGGGAGGCTGCTGGTGAATATTAACCAAGGTCACCACAGTTATCAGGGGATCAAACAGGGGCTAAGTCCA |
| | | C |
| 765 | 5214_Human_quadMut_37A_46A_48G_62A_n1 | GGGGGAGGCTGCTGGTGAATATTAACCAAGGTCACCACAGTTATCAGGGGAGCAAACAGGGACTAAGTCCA |
| | | C |
| 766 | 5216_Human_quadMut_37T_46A_52A_62T_n1 | GGGGGAGGCTGCTGGTGAATATTAACCAAGGTCACCTCAGTTATCAGAGGAACAAACAGGGTCTAAGTCCA |
| | | C |
| 767 | 5217_Human_quadMut_37T_46A_52T_72A_n1 | GGGGGAGGCTGCTGGTGAATATTAACCAAGGTCACCTCAGTTATCAGAGGATCAAACAGGGGCTAAGTCCA |
| | | A |
| 768 | 5219_Human_quadMut_37A_48G_52T_62C_n1 | GGGGGAGGCTGCTGGTGAATATTAACCAAGGTCACCACAGTTATCGGGGGATCAAACAGGGCCTAAGTCCA |
| | | C |
| 769 | 5222_Human_quadMut_37T_52A_62T_72A_n1 | GGGGGAGGCTGCTGGTGAATATTAACCAAGGTCACCTCAGTTATCGGAGGAACAAACAGGGTCTAAGTCCA |
| | | A |
| 770 | 5223_Human_quadMut_45G_46A_48G_52A_n1 | GGGGGAGGCTGCTGGTGAATATTAACCAAGGTCACCCCAGTTATGAGGGGAACAAACAGGGGCTAAGTCCA |
| | | C |
| 771 | 5224_Human_quadMut_45T_46T_48T_62C_n1 | GGGGGAGGCTGCTGGTGAATATTAACCAAGGTCACCCCAGTTATTTGTGGAGCAAACAGGGCCTAAGTCCA |
| | | C |
| 772 | 5228_Human_quadMut_45G_46A_62T_72A_n1 | GGGGGAGGCTGCTGGTGAATATTAACCAAGGTCACCCCAGTTATGAGAGGAGCAAACAGGGTCTAAGTCCA |
| | | A |
| 773 | 5230_Human_quadMut_45A_48G_52T_72A_n1 | GGGGGAGGCTGCTGGTGAATATTAACCAAGGTCACCCCAGTTATAGGGGGATCAAACAGGGGCTAAGTCCA |
| | | A |
| 774 | 5232_Human_quadMut_45G_52T_62C_72T_n1 | GGGGGAGGCTGCTGGTGAATATTAACCAAGGTCACCCCAGTTATGGGAGGATCAAACAGGGCCTAAGTCCA |
| | | T |
| 775 | 5233_Human_quadMut_46A_48G_52C_62T_n1 | GGGGGAGGCTGCTGGTGAATATTAACCAAGGTCACCCCAGTTATCAGGGGACCAAACAGGGTCTAAGTCCA |
| | | C |
| 776 | 5235_Human_quadMut_46T_48G_62A_72T_n1 | GGGGGAGGCTGCTGGTGAATATTAACCAAGGTCACCCCAGTTATCTGGGGAGCAAACAGGGACTAAGTCCA |
| | | T |
| 777 | 5240_Human_quadMut_1T_3A_8A_46T_n1 | TGAGGAGACTGCTGGTGAATATTAACCAAGGTCACCCCAGTTATCTGAGGAGCAAACAGGGGCTAAGTCCA |
| | | C |
| 778 | 5241_Human_quadMut_1A_3A_8T_48T_n1 | AGAGGAGTCTGCTGGTGAATATTAACCAAGGTCACCCCAGTTATCGGTGGAGCAAACAGGGGCTAAGTCCA |
| | | C |
| 779 | 5242_Human_quadMut_1A_3A_8A_52T_n1 | AGAGGAGACTGCTGGTGAATATTAACCAAGGTCACCCCAGTTATCGGAGGATCAAACAGGGGCTAAGTCCA |
| | | C |
| 780 | 5243_Human_quadMut_1A_3A_8C_62C_n1 | AGAGGAGCCTGCTGGTGAATATTAACCAAGGTCACCCCAGTTATCGGAGGAGCAAACAGGGCCTAAGTCCA |
| | | C |
| 781 | 5245_Human_quadMut_1A_3T_14A_35G_n1 | AGTGGAGGCTGCTAGTGAATATTAACCAAGGTCAGCCCAGTTATCGGAGGAGCAAACAGGGGCTAAGTCCA |
| | | C |
| 782 | 5248_Human_quadMut_1T_3A_14A_46T_n1 | TGAGGAGGCTGCTAGTGAATATTAACCAAGGTCACCCCAGTTATCTGAGGAGCAAACAGGGGCTAAGTCCA |
| | | C |
| 783 | 5252_Human_quadMut_1A_3T_14A_72A_n1 | AGTGGAGGCTGCTAGTGAATATTAACCAAGGTCACCCCAGTTATCGGAGGAGCAAACAGGGGCTAAGTCCA |
| | | A |
| 784 | 5260_Human_quadMut_1A_3A_37A_46A_n1 | AGAGGAGGCTGCTGGTGAATATTAACCAAGGTCACCACAGTTATCAGAGGAGCAAACAGGGGCTAAGTCCA |
| | | C |
| 785 | 5262_Human_quadMut_1A_3T_37A_62C_n1 | AGTGGAGGCTGCTGGTGAATATTAACCAAGGTCACCACAGTTATCGGAGGAGCAAACAGGGCCTAAGTCCA |
| | | C |
| 786 | 5263_Human_quadMut_1A_3T_37T_72A_n1 | AGTGGAGGCTGCTGGTGAATATTAACCAAGGTCACCTCAGTTATCGGAGGAGCAAACAGGGGCTAAGTCCA |
| | | A |
| 787 | 5269_Human_quadMut_1T_3A_46A_52A_n1 | TGAGGAGGCTGCTGGTGAATATTAACCAAGGTCACCCCAGTTATCAGAGGAACAAACAGGGGCTAAGTCCA |
| | | C |
| 788 | 5271_Human_quadMut_1A_3T_46T_72A_n1 | AGTGGAGGCTGCTGGTGAATATTAACCAAGGTCACCCCAGTTATCTGAGGAGCAAACAGGGGCTAAGTCCA |
| | | A |
| 789 | 5274_Human_quadMut_1A_3A_48G_72A_n1 | AGAGGAGGCTGCTGGTGAATATTAACCAAGGTCACCCCAGTTATCGGGGGAGCAAACAGGGGCTAAGTCCA |
| | | A |
| 790 | 5275_Human_quadMut_1T_3A_52T_62C_n1 | TGAGGAGGCTGCTGGTGAATATTAACCAAGGTCACCCCAGTTATCGGAGGATCAAACAGGGCCTAAGTCCA |
| | | C |
| 791 | 5283_Human_quadMut_1A_8C_14A_52A_n1 | AGGGGAGCCTGCTAGTGAATATTAACCAAGGTCACCCCAGTTATCGGAGGAACAAACAGGGGCTAAGTCCA |
| | | C |
| 792 | 5286_Human_quadMut_1A_8A_35G_37A_n1 | AGGGGAGACTGCTGGTGAATATTAACCAAGGTCAGCACAGTTATCGGAGGAGCAAACAGGGGCTAAGTCCA |
| | | C |
| 793 | 5287_Human_quadMut_1T_8T_35A_45G_n1 | TGGGGAGTCTGCTGGTGAATATTAACCAAGGTCAACCCAGTTATGGGAGGAGCAAACAGGGGCTAAGTCCA |
| | | C |
| 794 | 5289_Human_quadMut_1A_8C_35A_48T_n1 | AGGGGAGCCTGCTGGTGAATATTAACCAAGGTCAACCCAGTTATCGGTGGAGCAAACAGGGGCTAAGTCCA |
| | | C |
| 795 | 5296_Human_quadMut_1A_8C_37T_62C_n1 | AGGGGAGCCTGCTGGTGAATATTAACCAAGGTCACCTCAGTTATCGGAGGAGCAAACAGGGCCTAAGTCCA |
| | | C |
| 796 | 5304_Human_quadMut_1A_8A_46A_52T_n1 | AGGGGAGACTGCTGGTGAATATTAACCAAGGTCACCCCAGTTATCAGAGGATCAAACAGGGGCTAAGTCCA |
| | | C |
| 797 | 5313_Human_quadMut_1T_14T_35T_37T_n1 | TGGGGAGGCTGCTTGTGAATATTAACCAAGGTCATCTCAGTTATCGGAGGAGCAAACAGGGGCTAAGTCCA |
| | | C |
| 798 | 5322_Human_quadMut_1A_14A_37A_52T_n1 | AGGGGAGGCTGCTAGTGAATATTAACCAAGGTCACCACAGTTATCGGAGGATCAAACAGGGGCTAAGTCCA |
| | | C |
| 799 | 5324_Human_quadMut_1A_14A_37T_72T_n1 | AGGGGAGGCTGCTAGTGAATATTAACCAAGGTCACCTCAGTTATCGGAGGAGCAAACAGGGGCTAAGTCCA |
| | | T |
| 800 | 5331_Human_quadMut_1T_14T_46T_52T_n1 | TGGGGAGGCTGCTTGTGAATATTAACCAAGGTCACCCCAGTTATCTGAGGATCAAACAGGGGCTAAGTCCA |
| | | C |
| 801 | 5332_Human_quadMut_1A_14A_46A_62C_n1 | AGGGGAGGCTGCTAGTGAATATTAACCAAGGTCACCCCAGTTATCAGAGGAGCAAACAGGGCCTAAGTCCA |
| | | C |
| 802 | 5335_Human_quadMut_1T_14T_52A_62A_n1 | TGGGGAGGCTGCTTGTGAATATTAACCAAGGTCACCCCAGTTATCGGAGGAACAAACAGGGACTAAGTCCA |
| | | C |
| 803 | 5338_Human_quadMut_1T_35G_37A_45T_n1 | TGGGGAGGCTGCTGGTGAATATTAACCAAGGTCAGCACAGTTATTGGAGGAGCAAACAGGGGCTAAGTCCA |
| | | C |
| 804 | 5339_Human_quadMut_1A_35T_37T_46T_n1 | AGGGGAGGCTGCTGGTGAATATTAACCAAGGTCATCTCAGTTATCTGAGGAGCAAACAGGGGCTAAGTCCA |
| | | C |
| 805 | 5342_Human_quadMut_1A_35G_37T_62C_n1 | AGGGGAGGCTGCTGGTGAATATTAACCAAGGTCAGCTCAGTTATCGGAGGAGCAAACAGGGCCTAAGTCCA |
| | | C |
| 806 | 5348_Human_quadMut_1T_35G_46T_48T_n1 | TGGGGAGGCTGCTGGTGAATATTAACCAAGGTCAGCCCAGTTATCTGTGGAGCAAACAGGGGCTAAGTCCA |
| | | C |
| 807 | 5349_Human_quadMut_1A_35G_46A_52T_n1 | AGGGGAGGCTGCTGGTGAATATTAACCAAGGTCAGCCCAGTTATCAGAGGATCAAACAGGGGCTAAGTCCA |
| | | C |
| 808 | 5350_Human_quadMut_1T_35G_46A_72T_n1 | TGGGGAGGCTGCTGGTGAATATTAACCAAGGTCAGCCCAGTTATCAGAGGAGCAAACAGGGGCTAAGTCCA |
| | | T |
| 809 | 5353_Human_quadMut_1T_35G_48G_72T_n1 | TGGGGAGGCTGCTGGTGAATATTAACCAAGGTCAGCCCAGITATCGGGGGAGCAAACAGGGGCTAAGTCCA |
| | | T |
| 810 | 5354_Human_quadMut_1A_35A_52C_62C_n1 | AGGGGAGGCTGCTGGTGAATATTAACCAAGGTCAACCCAGTTATCGGAGGACCAAACAGGGCCTAAGTCCA |
| | | C |
| 811 | 5356_Human_quadMut_1A_35T_62C_72A_n1 | AGGGGAGGCTGCTGGTGAATATTAACCAAGGTCATCCCAGTTATCGGAGGAGCAAACAGGGCCTAAGTCCA |
| | | A |
| 812 | 5358_Human_quadMut_1A_37A_45A_52T_n1 | AGGGGAGGCTGCTGGTGAATATTAACCAAGGTCACCACAGTTATAGGAGGATCAAACAGGGGCTAAGTCCA |
| | | C |
| 813 | 5361_Human_quadMut_1A_37A_46T_48T_n1 | AGGGGAGGCTGCTGGTGAATATTAACCAAGGTCACCACAGTTATCTGTGGAGCAAACAGGGGCTAAGTCCA |
| | | C |
| 814 | 5362_Human_quadMut_1T_37T_46T_52A_n1 | TGGGGAGGCTGCTGGTGAATATTAACCAAGGTCACCTCAGTTATCTGAGGAACAAACAGGGGCTAAGTCCA |
| | | C |
| 815 | 5372_Human_quadMut_1A_45G_46A_52A_n1 | AGGGGAGGCTGCTGGTGAATATTAACCAAGGTCACCCCAGTTATGAGAGGAACAAACAGGGGCTAAGTCCA |
| | | C |
| 816 | 5377_Human_quadMut_1A_45G_48G_72T_n1 | AGGGGAGGCTGCTGGTGAATATTAACCAAGGTCACCCCAGTTATGGGGGGAGCAAACAGGGGCTAAGTCCA |
| | | T |
| 817 | 5378_Human_quadMut_1T_45A_52A_62T_n1 | TGGGGAGGCTGCTGGTGAATATTAACCAAGGTCACCCCAGTTATAGGAGGAACAAACAGGGTCTAAGTCCA |
| | | C |
| 818 | 5381_Human_quadMut_1A_46A_48G_62T_n1 | AGGGGAGGCTGCTGGTGAATATTAACCAAGGTCACCCCAGTTATCAGGGGAGCAAACAGGGTCTAAGTCCA |
| | | C |
| 819 | 5382_Human_quadMut_1A_46A_48G_72A_n1 | AGGGGAGGCTGCTGGTGAATATTAACCAAGGTCACCCCAGTTATCAGGGGAGCAAACAGGGGCTAAGTCCA |
| | | A |
| 820 | 5384_Human_quadMut_1T_46T_52T_72A_n1 | TGGGGAGGCTGCTGGTGAATATTAACCAAGGTCACCCCAGTTATCTGAGGATCAAACAGGGGCTAAGTCCA |
| | | A |
| 821 | 5386_Human_quadMut_1A_48G_52T_62C_n1 | AGGGGAGGCTGCTGGTGAATATTAACCAAGGTCACCCCAGTTATCGGGGGATCAAACAGGGCCTAAGTCCA |
| | | C |
| 822 | 5388_Human_quadMut_1A_48G_62C_72T_n1 | AGGGGAGGCTGCTGGTGAATATTAACCAAGGTCACCCCAGTTATCGGGGGAGCAAACAGGGCCTAAGTCCA |
| | | T |
| 823 | 5392_Human_quadMut_3A_8A_14T_45G_n1 | GGAGGAGACTGCTTGTGAATATTAACCAAGGTCACCCCAGTTATGGGAGGAGCAAACAGGGGCTAAGTCCA |
| | | C |
| 824 | 5398_Human_quadMut_3A_8A_35T_37T_n1 | GGAGGAGACTGCTGGTGAATATTAACCAAGGTCATCTCAGTTATCGGAGGAGCAAACAGGGGCTAAGTCCA |
| | | C |
| 825 | 5406_Human_quadMut_3A_8C_37T_46A_n1 | GGAGGAGCCTGCTGGTGAATATTAACCAAGGTCACCTCAGTTATCAGAGGAGCAAACAGGGGCTAAGTCCA |
| | | C |
| 826 | 5407_Human_quadMut_3T_8C_37T_48G_n1 | GGTGGAGCCTGCTGGTGAATATTAACCAAGGTCACCTCAGTTATCGGGGGAGCAAACAGGGGCTAAGTCCA |
| | | C |
| 827 | 5408_Human_quadMut_3T_8A_37T_52T_n1 | GGTGGAGACTGCTGGTGAATATTAACCAAGGTCACCTCAGTTATCGGAGGATCAAACAGGGGCTAAGTCCA |
| | | C |
| 828 | 5409_Human_quadMut_3T_8T_37A_62A_n1 | GGTGGAGTCTGCTGGTGAATATTAACCAAGGTCACCACAGTTATCGGAGGAGCAAACAGGGACTAAGTCCA |
| | | C |
| 829 | 5423_Human_quadMut_3A_8A_52T_62A_n1 | GGAGGAGACTGCTGGTGAATATTAACCAAGGTCACCCCAGTTATCGGAGGATCAAACAGGGACTAAGTCCA |
| | | C |
| 830 | 5424_Human_quadMut_3A_8A_52T_72G_n1 | GGAGGAGACTGCTGGTGAATATTAACCAAGGTCACCCCAGTTATCGGAGGATCAAACAGGGGCTAAGTCCA |
| | | G |
| 831 | 5425_Human_quadMut_3A_8T_62T_72A_n1 | GGAGGAGTCTGCTGGTGAATATTAACCAAGGTCACCCCAGTTATCGGAGGAGCAAACAGGGTCTAAGTCCA |
| | | A |
| 832 | 5430_Human_quadMut_3A_14T_35G_52T_n1 | GGAGGAGGCTGCTTGTGAATATTAACCAAGGTCAGCCCAGTTATCGGAGGATCAAACAGGGGCTAAGTCCA |
| | | C |
| 833 | 5435_Human_quadMut_3T_14T_37A_48G_n1 | GGTGGAGGCTGCTTGTGAATATTAACCAAGGTCACCACAGTTATCGGGGGAGCAAACAGGGGCTAAGTCCA |
| | | C |
| 834 | 5436_Human_quadMut_3A_14A_37T_52T_n1 | GGAGGAGGCTGCTAGTGAATATTAACCAAGGTCACCTCAGTTATCGGAGGATCAAACAGGGGCTAAGTCCA |
| | | C |
| 835 | 5443_Human_quadMut_3T_14A_45T_72A_n1 | GGTGGAGGCTGCTAGTGAATATTAACCAAGGTCACCCCAGTTATTGGAGGAGCAAACAGGGGCTAAGTCCA |
| | | A |
| 836 | 5444_Human_quadMut_3A_14A_46A_48T_n1 | GGAGGAGGCTGCTAGTGAATATTAACCAAGGTCACCCCAGTTATCAGTGGAGCAAACAGGGGCTAAGTCCA |
| | | C |
| 837 | 5447_Human_quadMut_3A_14A_46A_72A_n1 | GGAGGAGGCTGCTAGTGAATATTAACCAAGGTCACCCCAGTTATCAGAGGAGCAAACAGGGGCTAAGTCCA |
| | | A |
| 838 | 5450_Human_quadMut_3A_14T_48T_72G_n1 | GGAGGAGGCTGCTTGTGAATATTAACCAAGGTCACCCCAGTTATCGGTGGAGCAAACAGGGGCTAAGTCCA |
| | | G |
| 839 | 5451_Human_quadMut_3A_14A_52A_62C_n1 | GGAGGAGGCTGCTAGTGAATATTAACCAAGGTCACCCCAGTTATCGGAGGAACAAACAGGGCCTAAGTCCA |
| | | C |
| 840 | 5455_Human_quadMut_3A_35A_37A_46A_n1 | GGAGGAGGCTGCTGGTGAATATTAACCAAGGTCAACACAGTTATCAGAGGAGCAAACAGGGGCTAAGTCCA |
| | | C |
| 841 | 5456_Human_quadMut_3A_35A_37A_48G_n1 | GGAGGAGGCTGCTGGTGAATATTAACCAAGGTCAACACAGTTATCGGGGGAGCAAACAGGGGCTAAGTCCA |
| | | C |
| 842 | 5457_Human_quadMut_3T_35T_37T_52C_n1 | GGTGGAGGCTGCTGGTGAATATTAACCAAGGTCATCTCAGTTATCGGAGGACCAAACAGGGGCTAAGTCCA |
| | | C |
| 843 | 5465_Human_quadMut_3T_35T_46A_48G_n1 | GGTGGAGGCTGCTGGTGAATATTAACCAAGGTCATCCCAGTTATCAGGGGAGCAAACAGGGGCTAAGTCCA |
| | | C |
| 844 | 5467_Human_quadMut_3T_35A_46A_62C_n1 | GGTGGAGGCTGCTGGTGAATATTAACCAAGGTCAACCCAGTTATCAGAGGAGCAAACAGGGCCTAAGTCCA |
| | | C |
| 845 | 5468_Human_quadMut_3A_35A_46T_72T_n1 | GGAGGAGGCTGCTGGTGAATATTAACCAAGGTCAACCCAGTTATCTGAGGAGCAAACAGGGGCTAAGTCCA |
| | | T |
| 846 | 5481_Human_quadMut_3T_37T_46T_52T_n1 | GGTGGAGGCTGCTGGTGAATATTAACCAAGGTCACCTCAGTTATCTGAGGATCAAACAGGGGCTAAGTCCA |
| | | C |
| 847 | 5482_Human_quadMut_3T_37T_46A_62T_n1 | GGTGGAGGCTGCTGGTGAATATTAACCAAGGTCACCTCAGTTATCAGAGGAGCAAACAGGGTCTAAGTCCA |
| | | C |
| 848 | 5485_Human_quadMut_3A_37A_48G_62C_n1 | GGAGGAGGCTGCTGGTGAATATTAACCAAGGTCACCACAGTTATCGGGGGAGCAAACAGGGCCTAAGTCCA |
| | | C |
| 849 | 5487_Human_quadMut_3T_37A_52T_62C_n1 | GGTGGAGGCTGCTGGTGAATATTAACCAAGGTCACCACAGTTATCGGAGGATCAAACAGGGCCTAAGTCCA |
| | | C |
| 850 | 5488_Human_quadMut_3A_37A_52C_72A_n1 | GGAGGAGGCTGCTGGTGAATATTAACCAAGGTCACCACAGTTATCGGAGGACCAAACAGGGGCTAAGTCCA |
| | | A |
| 851 | 5491_Human_quadMut_3A_45G_46T_52C_n1 | GGAGGAGGCTGCTGGTGAATATTAACCAAGGTCACCCCAGTTATGTGAGGACCAAACAGGGGCTAAGTCCA |
| | | C |
| 852 | 5492_Human_quadMut_3A_45A_46A_62T_n1 | GGAGGAGGCTGCTGGTGAATATTAACCAAGGTCACCCCAGTTATAAGAGGAGCAAACAGGGTCTAAGTCCA |
| | | C |
| 853 | 5500_Human_quadMut_3A_46T_48G_52T_n1 | GGAGGAGGCTGCTGGTGAATATTAACCAAGGTCACCCCAGTTATCTGGGGATCAAACAGGGGCTAAGTCCA |
| | | C |
| 854 | 5503_Human_quadMut_3A_46A_52T_62C_n1 | GGAGGAGGCTGCTGGTGAATATTAACCAAGGTCACCCCAGTTATCAGAGGATCAAACAGGGCCTAAGTCCA |
| | | C |
| 855 | 5505_Human_quadMut_3A_46A_62C_72A_n1 | GGAGGAGGCTGCTGGTGAATATTAACCAAGGTCACCCCAGTTATCAGAGGAGCAAACAGGGCCTAAGTCCA |
| | | A |
| 856 | 5509_Human_quadMut_8A_14A_35A_37T_n1 | GGGGGAGACTGCTAGTGAATATTAACCAAGGTCAACTCAGTTATCGGAGGAGCAAACAGGGGCTAAGTCCA |
| | | C |
| 857 | 5513_Human_quadMut_8C_14T_35A_62T_n1 | GGGGGAGCCTGCTTGTGAATATTAACCAAGGTCAACCCAGTTATCGGAGGAGCAAACAGGGTCTAAGTCCA |
| | | C |
| 858 | 5517_Human_quadMut_8T_14T_37T_48G_n1 | GGGGGAGTCTGCTTGTGAATATTAACCAAGGTCACCTCAGTTATCGGGGGAGCAAACAGGGGCTAAGTCCA |
| | | C |
| 859 | 5519_Human_quadMut_8A_14A_37A_62A_n1 | GGGGGAGACTGCTAGTGAATATTAACCAAGGTCACCACAGTTATCGGAGGAGCAAACAGGGACTAAGTCCA |
| | | C |
| 860 | 5526_Human_quadMut_8C_14T_46A_48T_n1 | GGGGGAGCCTGCTTGTGAATATTAACCAAGGTCACCCCAGTTATCAGTGGAGCAAACAGGGGCTAAGTCCA |
| | | C |
| 861 | 5527_Human_quadMut_8T_14T_46A_52T_n1 | GGGGGAGTCTGCTTGTGAATATTAACCAAGGTCACCCCAGTTATCAGAGGATCAAACAGGGGCTAAGTCCA |
| | | C |
| 862 | 5531_Human_quadMut_8C_14T_48T_62T_n1 | GGGGGAGCCTGCTTGTGAATATTAACCAAGGTCACCCCAGTTATCGGTGGAGCAAACAGGGTCTAAGTCCA |
| | | C |
| 863 | 5532_Human_quadMut_8C_14T_48G_72A_n1 | GGGGGAGCCTGCTTGTGAATATTAACCAAGGTCACCCCAGTTATCGGGGGAGCAAACAGGGGCTAAGTCCA |
| | | A |
| 864 | 5533_Human_quadMut_8T_14T_52A_62C_n1 | GGGGGAGTCTGCTTGTGAATATTAACCAAGGTCACCCCAGTTATCGGAGGAACAAACAGGGCCTAAGTCCA |
| | | C |
| 865 | 5537_Human_quadMut_8A_35A_37A_46A_n1 | GGGGGAGACTGCTGGTGAATATTAACCAAGGTCAACACAGTTATCAGAGGAGCAAACAGGGGCTAAGTCCA |
| | | C |
| 866 | 5540_Human_quadMut_8C_35T_37T_62A_n1 | GGGGGAGCCTGCTGGTGAATATTAACCAAGGTCATCTCAGTTATCGGAGGAGCAAACAGGGACTAAGTCCA |
| | | C |
| 867 | 5546_Human_quadMut_8C_35T_46A_48T_n1 | GGGGGAGCCTGCTGGTGAATATTAACCAAGGTCATCCCAGTTATCAGTGGAGCAAACAGGGGCTAAGTCCA |
| | | C |
| 868 | 5547_Human_quadMut_8C_35G_46A_52A_n1 | GGGGGAGCCTGCTGGTGAATATTAACCAAGGTCAGCCCAGTTATCAGAGGAACAAACAGGGGCTAAGTCCA |
| | | C |
| 869 | 5548_Human_quadMut_8T_35A_46A_62T_n1 | GGGGGAGTCTGCTGGTGAATATTAACCAAGGTCAACCCAGTTATCAGAGGAGCAAACAGGGTCTAAGTCCA |
| | | C |
| 870 | 5551_Human_quadMut_8C_35T_48G_62T_n1 | GGGGGAGCCTGCTGGTGAATATTAACCAAGGTCATCCCAGTTATCGGGGGAGCAAACAGGGTCTAAGTCCA |
| | | C |
| 872 | 5553_Human_quadMut_8C_35A_52T_62A_n1 | GGGGGAGCCTGCTGGTGAATATTAACCAAGGTCAACCCAGTTATCGGAGGATCAAACAGGGACTAAGTCCA |
| | | C |
| 873 | 5554_Human_quadMut_8A_35T_52A_72A_n1 | GGGGGAGACTGCTGGTGAATATTAACCAAGGTCATCCCAGTTATCGGAGGAACAAACAGGGGCTAAGTCCA |
| | | A |
| 874 | 5556_Human_quadMut_8A_37T_45T_46A_n1 | GGGGGAGACTGCTGGTGAATATTAACCAAGGTCACCTCAGTTATTAGAGGAGCAAACAGGGGCTAAGTCCA |
| | | C |
| 875 | 5557_Human_quadMut_8C_37T_45G_48T_n1 | GGGGGAGCCTGCTGGTGAATATTAACCAAGGTCACCTCAGTTATGGGTGGAGCAAACAGGGGCTAAGTCCA |
| | | C |
| 876 | 5558_Human_quadMut_8A_37A_45G_52A_n1 | GGGGGAGACTGCTGGTGAATATTAACCAAGGTCACCACAGTTATGGGAGGAACAAACAGGGGCTAAGTCCA |
| | | C |
| 877 | 5561_Human_quadMut_8C_37A_46T_48G_n1 | GGGGGAGCCTGCTGGTGAATATTAACCAAGGTCACCACAGTTATCTGGGGAGCAAACAGGGGCTAAGTCCA |
| | | C |
| 878 | 5562_Human_quadMut_8C_37A_46T_52T_n1 | GGGGGAGCCTGCTGGTGAATATTAACCAAGGTCACCACAGTTATCTGAGGATCAAACAGGGGCTAAGTCCA |
| | | C |
| 879 | 5563_Human_quadMut_8C_37T_46A_62T_n1 | GGGGGAGCCTGCTGGTGAATATTAACCAAGGTCACCTCAGTTATCAGAGGAGCAAACAGGGTCTAAGTCCA |
| | | C |
| 880 | 5566_Human_quadMut_8T_37T_48G_62C_n1 | GGGGGAGTCTGCTGGTGAATATTAACCAAGGTCACCTCAGTTATCGGGGGAGCAAACAGGGCCTAAGTCCA |
| | | C |
| 881 | 5567_Human_quadMut_8A_37A_52A_62T_n1 | GGGGGAGACTGCTGGTGAATATTAACCAAGGTCACCACAGTTATCGGAGGAACAAACAGGGTCTAAGTCCA |
| | | C |
| 882 | 5568_Human_quadMut_8C_37T_52A_72A_n1 | GGGGGAGCCTGCTGGTGAATATTAACCAAGGTCACCTCAGTTATCGGAGGAACAAACAGGGGCTAAGTCCA |
| | | A |
| 883 | 5569_Human_quadMut_8A_37T_62T_72A_n1 | GGGGGAGACTGCTGGTGAATATTAACCAAGGTCACCTCAGTTATCGGAGGAGCAAACAGGGTCTAAGTCCA |
| | | A |
| 884 | 5572_Human_quadMut_8T_45T_46T_72A_n1 | GGGGGAGTCTGCTGGTGAATATTAACCAAGGTCACCCCAGTTATTTGAGGAGCAAACAGGGGCTAAGTCCA |
| | | A |
| 885 | 5573_Human_quadMut_8T_45G_48T_52A_n1 | GGGGGAGTCTGCTGGTGAATATTAACCAAGGTCACCCCAGTTATGGGTGGAACAAACAGGGGCTAAGTCCA |
| | | C |
| 886 | 5576_Human_quadMut_8C_45T_52T_62A_n1 | GGGGGAGCCTGCTGGTGAATATTAACCAAGGTCACCCCAGTTATTGGAGGATCAAACAGGGACTAAGTCCA |
| | | C |
| 887 | 5580_Human_quadMut_8C_46T_48G_62T_n1 | GGGGGAGCCTGCTGGTGAATATTAACCAAGGTCACCCCAGTTATCTGGGGAGCAAACAGGGTCTAAGTCCA |
| | | C |
| 888 | 5581_Human_quadMut_8A_46T_52C_62C_n1 | GGGGGAGACTGCTGGTGAATATTAACCAAGGTCACCCCAGTTATCTGAGGACCAAACAGGGCCTAAGTCCA |
| | | C |
| 889 | 5584_Human_quadMut_8C_48G_52A_62T_n1 | GGGGGAGCCTGCTGGTGAATATTAACCAAGGTCACCCCAGTTATCGGGGGAACAAACAGGGTCTAAGTCCA |
| | | C |
| 890 | 5590_Human_quadMut_14A_35T_37T_48G_n1 | GGGGGAGGCTGCTAGTGAATATTAACCAAGGTCATCTCAGTTATCGGGGGAGCAAACAGGGGCTAAGTCCA |
| | | C |
| 891 | 5592_Human_quadMut_14A_35G_37T_62T_n1 | GGGGGAGGCTGCTAGTGAATATTAACCAAGGTCAGCTCAGTTATCGGAGGAGCAAACAGGGTCTAAGTCCA |
| | | C |
| 892 | 5594_Human_quadMut_14A_35G_45G_46T_n1 | GGGGGAGGCTGCTAGTGAATATTAACCAAGGTCAGCCCAGTTATGTGAGGAGCAAACAGGGGCTAAGTCCA |
| | | C |
| 893 | 5599_Human_quadMut_14A_35A_46A_48G_n1 | GGGGGAGGCTGCTAGTGAATATTAACCAAGGTCAACCCAGTTATCAGGGGAGCAAACAGGGGCTAAGTCCA |
| | | C |
| 894 | 5601_Human_quadMut_14A_35T_46T_62C_n1 | GGGGGAGGCTGCTAGTGAATATTAACCAAGGTCATCCCAGTTATCTGAGGAGCAAACAGGGCCTAAGTCCA |
| | | C |
| 895 | 5604_Human_quadMut_14A_35A_48G_62T_n1 | GGGGGAGGCTGCTAGTGAATATTAACCAAGGTCAACCCAGTTATCGGGGGAGCAAACAGGGTCTAAGTCCA |
| | | C |
| 896 | 5606_Human_quadMut_14T_35A_52A_62C_n1 | GGGGGAGGCTGCTTGTGAATATTAACCAAGGTCAACCCAGTTATCGGAGGAACAAACAGGGCCTAAGTCCA |
| | | C |
| 897 | 5608_Human_quadMut_14A_35A_62T_72T_n1 | GGGGGAGGCTGCTAGTGAATATTAACCAAGGTCAACCCAGTTATCGGAGGAGCAAACAGGGTCTAAGTCCA |
| | | T |
| 898 | 5618_Human_quadMut_14A_37T_52A_62T_n1 | GGGGGAGGCTGCTAGTGAATATTAACCAAGGTCACCTCAGTTATCGGAGGAACAAACAGGGTCTAAGTCCA |
| | | C |
| 899 | 5633_Human_quadMut_14A_46T_48G_72T_n1 | GGGGGAGGCTGCTAGTGAATATTAACCAAGGTCACCCCAGTTATCTGGGGAGCAAACAGGGGCTAAGTCCA |
| | | T |
| 900 | 5634_Human_quadMut_14A_46A_52C_62C_n1 | GGGGGAGGCTGCTAGTGAATATTAACCAAGGTCACCCCAGTTATCAGAGGACCAAACAGGGCCTAAGTCCA |
| | | C |
| 901 | 5635_Human_quadMut_14A_46T_52T_72A_n1 | GGGGGAGGCTGCTAGTGAATATTAACCAAGGTCACCCCAGTTATCTGAGGATCAAACAGGGGCTAAGTCCA |
| | | A |
| 902 | 5640_Human_quadMut_35T_37T_45T_46A_n1 | GGGGGAGGCTGCTGGTGAATATTAACCAAGGTCATCTCAGTTATTAGAGGAGCAAACAGGGGCTAAGTCCA |
| | | C |
| 903 | 5641_Human_quadMut_35T_37A_45A_48T_n1 | GGGGGAGGCTGCTGGTGAATATTAACCAAGGTCATCACAGTTATAGGTGGAGCAAACAGGGGCTAAGTCCA |
| | | C |
| 904 | 5642_Human_quadMut_35T_37T_45T_52T_n1 | GGGGGAGGCTGCTGGTGAATATTAACCAAGGTCATCTCAGTTATTGGAGGATCAAACAGGGGCTAAGTCCA |
| | | C |
| 905 | 5645_Human_quadMut_35A_37A_46T_48G_n1 | GGGGGAGGCTGCTGGTGAATATTAACCAAGGTCAACACAGTTATCTGGGGAGCAAACAGGGGCTAAGTCCA |
| | | C |
| 906 | 5646_Human_quadMut_35G_37A_46A_52C_n1 | GGGGGAGGCTGCTGGTGAATATTAACCAAGGTCAGCACAGTTATCAGAGGACCAAACAGGGGCTAAGTCCA |
| | | C |
| 907 | 5647_Human_quadMut_35G_37T_46A_62T_n1 | GGGGGAGGCTGCTGGTGAATATTAACCAAGGTCAGCTCAGTTATCAGAGGAGCAAACAGGGTCTAAGTCCA |
| | | C |
| 908 | 5648_Human_quadMut_35A_37A_46T_72G_n1 | GGGGGAGGCTGCTGGTGAATATTAACCAAGGTCAACACAGTTATCTGAGGAGCAAACAGGGGCTAAGTCCA |
| | | G |
| 909 | 5649_Human_quadMut_35A_37A_48T_52T_n1 | GGGGGAGGCTGCTGGTGAATATTAACCAAGGTCAACACAGTTATCGGTGGATCAAACAGGGGCTAAGTCCA |
| | | C |
| 910 | 5652_Human_quadMut_35A_37A_52T_62A_n1 | GGGGGAGGCTGCTGGTGAATATTAACCAAGGTCAACACAGTTATCGGAGGATCAAACAGGGACTAAGTCCA |
| | | C |
| 911 | 5653_Human_quadMut_35A_37T_52T_72G_n1 | GGGGGAGGCTGCTGGTGAATATTAACCAAGGTCAACTCAGTTATCGGAGGATCAAACAGGGGCTAAGTCCA |
| | | G |
| 912 | 5654_Human_quadMut_35T_37T_62T_72T_n1 | GGGGGAGGCTGCTGGTGAATATTAACCAAGGTCATCTCAGTTATCGGAGGAGCAAACAGGGTCTAAGTCCA |
| | | T |
| 913 | 5655_Human_quadMut_35A_45T_46T_48T_n1 | GGGGGAGGCTGCTGGTGAATATTAACCAAGGTCAACCCAGTTATTTGTGGAGCAAACAGGGGCTAAGTCCA |
| | | C |
| 914 | 5656_Human_quadMut_35A_45T_46T_52T_n1 | GGGGGAGGCTGCTGGTGAATATTAACCAAGGTCAACCCAGTTATTTGAGGATCAAACAGGGGCTAAGTCCA |
| | | C |
| 915 | 5658_Human_quadMut_35G_45G_46A_72A_n1 | GGGGGAGGCTGCTGGTGAATATTAACCAAGGTCAGCCCAGTTATGAGAGGAGCAAACAGGGGCTAAGTCCA |
| | | A |
| 916 | 5660_Human_quadMut_35A_45A_48G_62T_n1 | GGGGGAGGCTGCTGGTGAATATTAACCAAGGTCAACCCAGTTATAGGGGGAGCAAACAGGGTCTAAGTCCA |
| | | C |
| 917 | 5661_Human_quadMut_35T_45T_48T_72A_n1 | GGGGGAGGCTGCTGGTGAATATTAACCAAGGTCATCCCAGTTATTGGTGGAGCAAACAGGGGCTAAGTCCA |
| | | A |
| 918 | 5662_Human_quadMut_35G_45T_52A_62T_n1 | GGGGGAGGCTGCTGGTGAATATTAACCAAGGTCAGCCCAGTTATTGGAGGAACAAACAGGGTCTAAGTCCA |
| | | C |
| 919 | 5665_Human_quadMut_35A_46A_48T_52A_n1 | GGGGGAGGCTGCTGGTGAATATTAACCAAGGTCAACCCAGTTATCAGTGGAACAAACAGGGGCTAAGTCCA |
| | | C |
| 920 | 5666_Human_quadMut_35A_46A_48G_62C_n1 | GGGGGAGGCTGCTGGTGAATATTAACCAAGGTCAACCCAGTTATCAGGGGAGCAAACAGGGCCTAAGTCCA |
| | | C |
| 921 | 5668_Human_quadMut_35A_46A_52T_62T_n1 | GGGGGAGGCTGCTGGTGAATATTAACCAAGGTCAACCCAGTTATCAGAGGATCAAACAGGGTCTAAGTCCA |
| | | C |
| 922 | 5669_Human_quadMut_35T_48G_52A_62T_n1 | GGGGGAGGCTGCTGGTGAATATTAACCAAGGTCATCCCAGTTATCGGGGGAACAAACAGGGTCTAAGTCCA |
| | | C |
| 923 | 5670_Human_quadMut_35G_48G_52C_72G_n1 | GGGGGAGGCTGCTGGTGAATATTAACCAAGGTCAGCCCAGTTATCGGGGGACCAAACAGGGGCTAAGTCCA |
| | | G |
| 924 | 5672_Human_quadMut_35A_52C_62T_72G_n1 | GGGGGAGGCTGCTGGTGAATATTAACCAAGGTCAACCCAGTTATCGGAGGACCAAACAGGGTCTAAGTCCA |
| | | G |
| 925 | 5674_Human_quadMut_37T_45G_46T_52A_n1 | GGGGGAGGCTGCTGGTGAATATTAACCAAGGTCACCTCAGTTATGTGAGGAACAAACAGGGGCTAAGTCCA |
| | | C |
| 926 | 5675_Human_quadMut_37A_45G_46A_62T_n1 | GGGGGAGGCTGCTGGTGAATATTAACCAAGGTCACCACAGTTATGAGAGGAGCAAACAGGGTCTAAGTCCA |
| | | C |
| 927 | 5683_Human_quadMut_37T_46A_48G_52A_n1 | GGGGGAGGCTGCTGGTGAATATTAACCAAGGTCACCTCAGTTATCAGGGGAACAAACAGGGGCTAAGTCCA |
| | | C |
| 928 | 5685_Human_quadMut_37A_46A_52C_62T_n1 | GGGGGAGGCTGCTGGTGAATATTAACCAAGGTCACCACAGTTATCAGAGGACCAAACAGGGTCTAAGTCCA |
| | | C |
| 929 | 5687_Human_quadMut_37T_46T_62T_72T_n1 | GGGGGAGGCTGCTGGTGAATATTAACCAAGGTCACCTCAGTTATCTGAGGAGCAAACAGGGTCTAAGTCCA |
| | | T |
| 930 | 5689_Human_quadMut_37T_48G_52T_72A_n1 | GGGGGAGGCTGCTGGTGAATATTAACCAAGGTCACCTCAGTTATCGGGGGATCAAACAGGGGCTAAGTCCA |
| | | A |
| 931 | 5690_Human_quadMut_37A_48G_62T_72A_n1 | GGGGGAGGCTGCTGGTGAATATTAACCAAGGTCACCACAGTTATCGGGGGAGCAAACAGGGTCTAAGTCCA |
| | | A |
| 932 | 5691_Human_quadMut_37T_52T_62A_72T_n1 | GGGGGAGGCTGCTGGTGAATATTAACCAAGGTCACCTCAGTTATCGGAGGATCAAACAGGGACTAAGTCCA |
| | | T |
| 933 | 5695_Human_quadMut_45G_46A_52A_62C_n1 | GGGGGAGGCTGCTGGTGAATATTAACCAAGGTCACCCCAGTTATGAGAGGAACAAACAGGGCCTAAGTCCA |
| | | C |
| 934 | 5696_Human_quadMut_45G_46A_52A_72G_n1 | GGGGGAGGCTGCTGGTGAATATTAACCAAGGTCACCCCAGTTATGAGAGGAACAAACAGGGGCTAAGTCCA |
| | | G |
| 935 | 5702_Human_quadMut_46A_48G_52A_62A_n1 | GGGGGAGGCTGCTGGTGAATATTAACCAAGGTCACCCCAGTTATCAGGGGAACAAACAGGGACTAAGTCCA |
| | | C |
| 936 | 5704_Human_quadMut_46T_48G_62T_72T_n1 | GGGGGAGGCTGCTGGTGAATATTAACCAAGGTCACCCCAGTTATCTGGGGAGCAAACAGGGTCTAAGTCCA |
| | | T |
| 937 | 5705_Human_quadMut_46T_52C_62T_72A_n1 | GGGGGAGGCTGCTGGTGAATATTAACCAAGGTCACCCCAGTTATCTGAGGACCAAACAGGGTCTAAGTCCA |
| | | A |
| 938 | 5708_Human_quadMut_1T_3T_8T_37T_n1 | TGTGGAGTCTGCTGGTGAATATTAACCAAGGTCACCTCAGTTATCGGAGGAGCAAACAGGGGCTAAGTCCA |
| | | C |
| 939 | 5712_Human_quadMut_1A_3T_8C_52A_n1 | AGTGGAGCCTGCTGGTGAATATTAACCAAGGTCACCCCAGTTATCGGAGGAACAAACAGGGGCTAAGTCCA |
| | | C |
| 940 | 5724_Human_quadMut_1A_3A_35T_72A_n1 | AGAGGAGGCTGCTGGTGAATATTAACCAAGGTCATCCCAGTTATCGGAGGAGCAAACAGGGGCTAAGTCCA |
| | | A |
| 941 | 5726_Human_quadMut_1T_3A_37A_48T_n1 | TGAGGAGGCTGCTGGTGAATATTAACCAAGGTCACCACAGTTATCGGTGGAGCAAACAGGGGCTAAGTCCA |
| | | C |
| 942 | 5728_Human_quadMut_1T_3A_37T_62C_n1 | TGAGGAGGCTGCTGGTGAATATTAACCAAGGTCACCTCAGTTATCGGAGGAGCAAACAGGGCCTAAGTCCA |
| | | C |
| 943 | 5730_Human_quadMut_1A_3A_45T_46T_n1 | AGAGGAGGCTGCTGGTGAATATTAACCAAGGTCACCCCAGTTATTTGAGGAGCAAACAGGGGCTAAGTCCA |
| | | C |
| 944 | 5733_Human_quadMut_1T_3A_45A_62T_n1 | TGAGGAGGCTGCTGGTGAATATTAACCAAGGTCACCCCAGTTATAGGAGGAGCAAACAGGGTCTAAGTCCA |
| | | C |
| 945 | 5736_Human_quadMut_1T_3T_46A_52A_n1 | TGTGGAGGCTGCTGGTGAATATTAACCAAGGTCACCCCAGTTATCAGAGGAACAAACAGGGGCTAAGTCCA |
| | | C |
| 946 | 5739_Human_quadMut_1T_3A_48T_52A_n1 | TGAGGAGGCTGCTGGTGAATATTAACCAAGGTCACCCCAGTTATCGGTGGAACAAACAGGGGCTAAGTCCA |
| | | C |
| 947 | 5740_Human_quadMut_1A_3T_48T_62T_n1 | AGTGGAGGCTGCTGGTGAATATTAACCAAGGTCACCCCAGTTATCGGTGGAGCAAACAGGGTCTAAGTCCA |
| | | C |
| 948 | 5743_Human_quadMut_1T_3T_52T_72G_n1 | TGTGGAGGCTGCTGGTGAATATTAACCAAGGTCACCCCAGTTATCGGAGGATCAAACAGGGGCTAAGTCCA |
| | | G |
| 949 | 5745_Human_quadMut_1A_8A_14T_37T_n1 | AGGGGAGACTGCTTGTGAATATTAACCAAGGTCACCTCAGTTATCGGAGGAGCAAACAGGGGCTAAGTCCA |
| | | C |
| 950 | 5754_Human_quadMut_1T_8A_35T_52T_n1 | TGGGGAGACTGCTGGTGAATATTAACCAAGGTCATCCCAGTTATCGGAGGATCAAACAGGGGCTAAGTCCA |
| | | C |
| 951 | 5759_Human_quadMut_1A_8A_37A_48T_n1 | AGGGGAGACTGCTGGTGAATATTAACCAAGGTCACCACAGTTATCGGTGGAGCAAACAGGGGCTAAGTCCA |
| | | C |
| 952 | 5760_Human_quadMut_1A_8C_37T_52A_n1 | AGGGGAGCCTGCTGGTGAATATTAACCAAGGTCACCTCAGTTATCGGAGGAACAAACAGGGGCTAAGTCCA |
| | | C |
| 953 | 5767_Human_quadMut_1A_8C_46T_48G_n1 | AGGGGAGCCTGCTGGTGAATATTAACCAAGGTCACCCCAGTTATCTGGGGAGCAAACAGGGGCTAAGTCCA |
| | | C |
| 954 | 5772_Human_quadMut_1T_8T_48G_62A_n1 | TGGGGAGTCTGCTGGTGAATATTAACCAAGGTCACCCCAGITATCGGGGGAGCAAACAGGGACTAAGTCCA |
| | | C |
| 955 | 5776_Human_quadMut_1A_8A_62T_72A_n1 | AGGGGAGACTGCTGGTGAATATTAACCAAGGTCACCCCAGTTATCGGAGGAGCAAACAGGGTCTAAGTCCA |
| | | A |
| 956 | 5779_Human_quadMut_1A_14T_35T_46A_n1 | AGGGGAGGCTGCTTGTGAATATTAACCAAGGTCATCCCAGTTATCAGAGGAGCAAACAGGGGCTAAGTCCA |
| | | C |
| 957 | 5781_Human_quadMut_1T_14A_35A_52A_n1 | TGGGGAGGCTGCTAGTGAATATTAACCAAGGTCAACCCAGTTATCGGAGGAACAAACAGGGGCTAAGTCCA |
| | | C |
| 958 | 5787_Human_quadMut_1T_14A_37T_52T_n1 | TGGGGAGGCTGCTAGTGAATATTAACCAAGGTCACCTCAGTTATCGGAGGATCAAACAGGGGCTAAGTCCA |
| | | C |
| 959 | 5791_Human_quadMut_1T_14A_45A_62T_n1 | TGGGGAGGCTGCTAGTGAATATTAACCAAGGTCACCCCAGTTATAGGAGGAGCAAACAGGGTCTAAGTCCA |
| | | C |
| 960 | 5793_Human_quadMut_1T_14A_46T_48T_n1 | TGGGGAGGCTGCTAGTGAATATTAACCAAGGTCACCCCAGTTATCTGTGGAGCAAACAGGGGCTAAGTCCA |
| | | C |
| 961 | 5794_Human_quadMut_1T_14A_46A_52T_n1 | TGGGGAGGCTGCTAGTGAATATTAACCAAGGTCACCCCAGTTATCAGAGGATCAAACAGGGGCTAAGTCCA |
| | | C |
| 962 | 5800_Human_quadMut_1T_14A_52T_62T_n1 | TGGGGAGGCTGCTAGTGAATATTAACCAAGGTCACCCCAGTTATCGGAGGATCAAACAGGGTCTAAGTCCA |
| | | C |
| 963 | 5805_Human_quadMut_1T_35G_37T_48G_n1 | TGGGGAGGCTGCTGGTGAATATTAACCAAGGTCAGCTCAGTTATCGGGGGAGCAAACAGGGGCTAAGTCCA |
| | | C |
| 964 | 5809_Human_quadMut_1A_35T_45T_46T_n1 | AGGGGAGGCTGCTGGTGAATATTAACCAAGGTCATCCCAGTTATTTGAGGAGCAAACAGGGGCTAAGTCCA |
| | | C |
| 965 | 5810_Human_quadMut_1A_35T_45G_48G_n1 | AGGGGAGGCTGCTGGTGAATATTAACCAAGGTCATCCCAGTTATGGGGGGAGCAAACAGGGGCTAAGTCCA |
| | | C |
| 966 | 5814_Human_quadMut_1T_35A_46T_48T_n1 | TGGGGAGGCTGCTGGTGAATATTAACCAAGGTCAACCCAGTTATCTGTGGAGCAAACAGGGGCTAAGTCCA |
| | | C |
| 967 | 5815_Human_quadMut_1A_35T_46A_52C_n1 | AGGGGAGGCTGCTGGTGAATATTAACCAAGGTCATCCCAGTTATCAGAGGACCAAACAGGGGCTAAGTCCA |
| | | C |
| 968 | 5816_Human_quadMut_1T_35A_46T_62T_n1 | TGGGGAGGCTGCTGGTGAATATTAACCAAGGTCAACCCAGTTATCTGAGGAGCAAACAGGGTCTAAGTCCA |
| | | C |
| 969 | 5817_Human_quadMut_1A_35A_46A_72T_n1 | AGGGGAGGCTGCTGGTGAATATTAACCAAGGTCAACCCAGTTATCAGAGGAGCAAACAGGGGCTAAGTCCA |
| | | T |
| 970 | 5821_Human_quadMut_1A_35G_52A_62T_n1 | AGGGGAGGCTGCTGGTGAATATTAACCAAGGTCAGCCCAGTTATCGGAGGAACAAACAGGGTCTAAGTCCA |
| | | C |
| 971 | 5823_Human_quadMut_1T_35T_62T_72G_n1 | TGGGGAGGCTGCTGGTGAATATTAACCAAGGTCATCCCAGTTATCGGAGGAGCAAACAGGGTCTAAGTCCA |
| | | G |
| 972 | 5824_Human_quadMut_1A_37A_45G_46A_n1 | AGGGGAGGCTGCTGGTGAATATTAACCAAGGTCACCACAGTTATGAGAGGAGCAAACAGGGGCTAAGTCCA |
| | | C |
| 973 | 5827_Human_quadMut_1A_37T_45G_62C_n1 | AGGGGAGGCTGCTGGTGAATATTAACCAAGGTCACCTCAGTTATGGGAGGAGCAAACAGGGCCTAAGTCCA |
| | | C |
| 974 | 5828_Human_quadMut_1T_37T_45G_72A_n1 | TGGGGAGGCTGCTGGTGAATATTAACCAAGGTCACCTCAGTTATGGGAGGAGCAAACAGGGGCTAAGTCCA |
| | | A |
| 975 | 5829_Human_quadMut_1A_37T_46A_52T_n1 | AGGGGAGGCTGCTGGTGAATATTAACCAAGGTCACCTCAGTTATCAGAGGATCAAACAGGGGCTAAGTCCA |
| | | C |
| 976 | 5832_Human_quadMut_1A_37A_48T_52T_n1 | AGGGGAGGCTGCTGGTGAATATTAACCAAGGTCACCACAGTTATCGGTGGATCAAACAGGGGCTAAGTCCA |
| | | C |
| 977 | 5839_Human_quadMut_1A_45G_46T_52T_n1 | AGGGGAGGCTGCTGGTGAATATTAACCAAGGTCACCCCAGTTATGTGAGGATCAAACAGGGGCTAAGTCCA |
| | | C |
| 978 | 5840_Human_quadMut_1T_45A_46T_62C_n1 | TGGGGAGGCTGCTGGTGAATATTAACCAAGGTCACCCCAGTTATATGAGGAGCAAACAGGGCCTAAGTCCA |
| | | C |
| 979 | 5848_Human_quadMut_1T_46T_48G_52C_n1 | TGGGGAGGCTGCTGGTGAATATTAACCAAGGTCACCCCAGTTATCTGGGGACCAAACAGGGGCTAAGTCCA |
| | | C |
| 980 | 5852_Human_quadMut_1T_46T_52T_72T_n1 | TGGGGAGGCTGCTGGTGAATATTAACCAAGGTCACCCCAGTTATCTGAGGATCAAACAGGGGCTAAGTCCA |
| | | T |
| 981 | 5855_Human_quadMut_1A_48G_52T_72T_n1 | AGGGGAGGCTGCTGGTGAATATTAACCAAGGTCACCCCAGTTATCGGGGGATCAAACAGGGGCTAAGTCCA |
| | | T |
| 982 | 5856_Human_quadMut_1T_48T_62C_72A_n1 | TGGGGAGGCTGCTGGTGAATATTAACCAAGGTCACCCCAGTTATCGGTGGAGCAAACAGGGCCTAAGTCCA |
| | | A |
| 983 | 5866_Human_quadMut_3T_8A_35T_46A_n1 | GGTGGAGACTGCTGGTGAATATTAACCAAGGTCATCCCAGTTATCAGAGGAGCAAACAGGGGCTAAGTCCA |
| | | C |
| 984 | 5867_Human_quadMut_3A_8A_35T_48T_n1 | GGAGGAGACTGCTGGTGAATATTAACCAAGGTCATCCCAGTTATCGGTGGAGCAAACAGGGGCTAAGTCCA |
| | | C |
| 985 | 5868_Human_quadMut_3A_8A_35T_52T_n1 | GGAGGAGACTGCTGGTGAATATTAACCAAGGTCATCCCAGTTATCGGAGGATCAAACAGGGGCTAAGTCCA |
| | | C |
| 986 | 5869_Human_quadMut_3A_8A_35A_62T_n1 | GGAGGAGACTGCTGGTGAATATTAACCAAGGTCAACCCAGTTATCGGAGGAGCAAACAGGGTCTAAGTCCA |
| | | C |
| 987 | 5880_Human_quadMut_3A_8T_45G_72A_n1 | GGAGGAGTCTGCTGGTGAATATTAACCAAGGTCACCCCAGTTATGGGAGGAGCAAACAGGGGCTAAGTCCA |
| | | A |
| 988 | 5882_Human_quadMut_3A_8A_46A_52A_n1 | GGAGGAGACTGCTGGTGAATATTAACCAAGGTCACCCCAGTTATCAGAGGAACAAACAGGGGCTAAGTCCA |
| | | C |
| 990 | 5883_Human_quadMut_3T_8T_46A_62T_n1 | GGTGGAGTCTGCTGGTGAATATTAACCAAGGTCACCCCAGTTATCAGAGGAGCAAACAGGGTCTAAGTCCA |
| | | C |
| 991 | 5884_Human_quadMut_3T_8C_46A_72A_n1 | GGTGGAGCCTGCTGGTGAATATTAACCAAGGTCACCCCAGTTATCAGAGGAGCAAACAGGGGCTAAGTCCA |
| | | A |
| 992 | 5886_Human_quadMut_3T_8T_48G_62T_n1 | GGTGGAGTCTGCTGGTGAATATTAACCAAGGTCACCCCAGTTATCGGGGGAGCAAACAGGGTCTAAGTCCA |
| | | C |
| 993 | 5889_Human_quadMut_3T_8A_52T_72G_n1 | GGTGGAGACTGCTGGTGAATATTAACCAAGGTCACCCCAGTTATCGGAGGATCAAACAGGGGCTAAGTCCA |
| | | G |
| 994 | 5897_Human_quadMut_3A_14A_37T_48T_n1 | GGAGGAGGCTGCTAGTGAATATTAACCAAGGTCACCTCAGTTATCGGTGGAGCAAACAGGGGCTAAGTCCA |
| | | C |
| 995 | 5899_Human_quadMut_3T_14A_37T_62T_n1 | GGTGGAGGCTGCTAGTGAATATTAACCAAGGTCACCTCAGTTATCGGAGGAGCAAACAGGGTCTAAGTCCA |
| | | C |
| 996 | 5905_Human_quadMut_3A_14T_45T_72A_n1 | GGAGGAGGCTGCTTGTGAATATTAACCAAGGTCACCCCAGTTATTGGAGGAGCAAACAGGGGCTAAGTCCA |
| | | A |
| 997 | 5907_Human_quadMut_3T_14A_46T_52T_n1 | GGTGGAGGCTGCTAGTGAATATTAACCAAGGTCACCCCAGTTATCTGAGGATCAAACAGGGGCTAAGTCCA |
| | | C |
| 998 | 5908_Human_quadMut_3T_14A_46A_62T_n1 | GGTGGAGGCTGCTAGTGAATATTAACCAAGGTCACCCCAGTTATCAGAGGAGCAAACAGGGTCTAAGTCCA |
| | | C |
| 999 | 5911_Human_quadMut_3T_14T_48T_62C_n1 | GGTGGAGGCTGCTTGTGAATATTAACCAAGGTCACCCCAGTTATCGGTGGAGCAAACAGGGCCTAAGTCCA |
| | | C |
| 1000 | 5914_Human_quadMut_3T_14A_52A_72A_n1 | GGTGGAGGCTGCTAGTGAATATTAACCAAGGTCACCCCAGTTATCGGAGGAACAAACAGGGGCTAAGTCCA |
| | | A |
| 1001 | 5916_Human_quadMut_3A_35A_37T_45G_n1 | GGAGGAGGCTGCTGGTGAATATTAACCAAGGTCAACTCAGTTATGGGAGGAGCAAACAGGGGCTAAGTCCA |
| | | C |
| 1002 | 5917_Human_quadMut_3T_35A_37A_46A_n1 | GGTGGAGGCTGCTGGTGAATATTAACCAAGGTCAACACAGTTATCAGAGGAGCAAACAGGGGCTAAGTCCA |
| | | C |
| 1003 | 5919_Human_quadMut_3A_35T_37A_52T_n1 | GGAGGAGGCTGCTGGTGAATATTAACCAAGGTCATCACAGTTATCGGAGGATCAAACAGGGGCTAAGTCCA |
| | | C |
| 1004 | 5921_Human_quadMut_3T_35G_45T_46T_n1 | GGTGGAGGCTGCTGGTGAATATTAACCAAGGTCAGCCCAGTTATTTGAGGAGCAAACAGGGGCTAAGTCCA |
| | | C |
| 1005 | 5926_Human_quadMut_3T_35T_46A_52C_n1 | GGTGGAGGCTGCTGGTGAATATTAACCAAGGTCATCCCAGTTATCAGAGGACCAAACAGGGGCTAAGTCCA |
| | | C |
| 1006 | 5927_Human_quadMut_3T_35A_46T_62T_n1 | GGTGGAGGCTGCTGGTGAATATTAACCAAGGTCAACCCAGTTATCTGAGGAGCAAACAGGGTCTAAGTCCA |
| | | C |
| 1007 | 5929_Human_quadMut_3A_35T_48G_52T_n1 | GGAGGAGGCTGCTGGTGAATATTAACCAAGGTCATCCCAGTTATCGGGGGATCAAACAGGGGCTAAGTCCA |
| | | C |
| 1008 | 5930_Human_quadMut_3T_35T_48T_62C_n1 | GGTGGAGGCTGCTGGTGAATATTAACCAAGGTCATCCCAGTTATCGGTGGAGCAAACAGGGCCTAAGTCCA |
| | | C |
| 1009 | 5932_Human_quadMut_3T_35G_52T_72G_n1 | GGTGGAGGCTGCTGGTGAATATTAACCAAGGTCAGCCCAGTTATCGGAGGATCAAACAGGGGCTAAGTCCA |
| | | G |
| 1010 | 5933_Human_quadMut_3T_35T_62A_72T_n1 | GGTGGAGGCTGCTGGTGAATATTAACCAAGGTCATCCCAGTTATCGGAGGAGCAAACAGGGACTAAGTCCA |
| | | T |
| 1011 | 5934_Human_quadMut_3T_37A_45G_46T_n1 | GGTGGAGGCTGCTGGTGAATATTAACCAAGGTCACCACAGTTATGTGAGGAGCAAACAGGGGCTAAGTCCA |
| | | C |
| 1012 | 5936_Human_quadMut_3T_37A_45G_52A_n1 | GGTGGAGGCTGCTGGTGAATATTAACCAAGGTCACCACAGTTATGGGAGGAACAAACAGGGGCTAAGTCCA |
| | | C |
| 1013 | 5941_Human_quadMut_3A_37T_48T_72T_n1 | GGAGGAGGCTGCTGGTGAATATTAACCAAGGTCACCTCAGTTATCGGTGGAGCAAACAGGGGCTAAGTCCA |
| | | T |
| 1014 | 5944_Human_quadMut_3A_37A_62T_72A_n1 | GGAGGAGGCTGCTGGTGAATATTAACCAAGGTCACCACAGTTATCGGAGGAGCAAACAGGGTCTAAGTCCA |
| | | A |
| 1015 | 5948_Human_quadMut_3A_45A_46A_72A_n1 | GGAGGAGGCTGCTGGTGAATATTAACCAAGGTCACCCCAGTTATAAGAGGAGCAAACAGGGGCTAAGTCCA |
| | | A |
| 1016 | 5950_Human_quadMut_3T_45A_48T_62T_n1 | GGTGGAGGCTGCTGGTGAATATTAACCAAGGTCACCCCAGTTATAGGTGGAGCAAACAGGGTCTAAGTCCA |
| | | C |
| 1017 | 5956_Human_quadMut_3T_46A_48T_72A_n1 | GGTGGAGGCTGCTGGTGAATATTAACCAAGGTCACCCCAGTTATCAGTGGAGCAAACAGGGGCTAAGTCCA |
| | | A |
| 1018 | 5962_Human_quadMut_3T_52T_62T_72T_n1 | GGTGGAGGCTGCTGGTGAATATTAACCAAGGTCACCCCAGTTATCGGAGGATCAAACAGGGTCTAAGTCCA |
| | | T |
| 1019 | 5963_Human_quadMut_8T_14A_35G_37T_n1 | GGGGGAGTCTGCTAGTGAATATTAACCAAGGTCAGCTCAGTTATCGGAGGAGCAAACAGGGGCTAAGTCCA |
| | | C |
| 1020 | 5968_Human_quadMut_8T_14T_35A_62T_n1 | GGGGGAGTCTGCTTGTGAATATTAACCAAGGTCAACCCAGTTATCGGAGGAGCAAACAGGGTCTAAGTCCA |
| | | C |
| 1021 | 5971_Human_quadMut_8T_14A_37A_46A_n1 | GGGGGAGTCTGCTAGTGAATATTAACCAAGGTCACCACAGTTATCAGAGGAGCAAACAGGGGCTAAGTCCA |
| | | C |
| 1022 | 5984_Human_quadMut_8A_14A_48G_52T_n1 | GGGGGAGACTGCTAGTGAATATTAACCAAGGTCACCCCAGTTATCGGGGGATCAAACAGGGGCTAAGTCCA |
| | | C |
| 1023 | 5985_Human_quadMut_8T_14T_48G_62A_n1 | GGGGGAGTCTGCTTGTGAATATTAACCAAGGTCACCCCAGTTATCGGGGGAGCAAACAGGGACTAAGTCCA |
| | | C |
| 1024 | 5990_Human_quadMut_8A_35T_37T_45G_n1 | GGGGGAGACTGCTGGTGAATATTAACCAAGGTCATCTCAGTTATGGGAGGAGCAAACAGGGGCTAAGTCCA |
| | | C |
| 1025 | 5991_Human_quadMut_8A_35G_37T_46T_n1 | GGGGGAGACTGCTGGTGAATATTAACCAAGGTCAGCTCAGTTATCTGAGGAGCAAACAGGGGCTAAGTCCA |
| | | C |
| 1026 | 5993_Human_quadMut_8C_35A_37T_52T_n1 | GGGGGAGCCTGCTGGTGAATATTAACCAAGGTCAACTCAGTTATCGGAGGATCAAACAGGGGCTAAGTCCA |
| | | C |
| 1027 | 5995_Human_quadMut_8A_35G_45A_46A_n1 | GGGGGAGACTGCTGGTGAATATTAACCAAGGTCAGCCCAGTTATAAGAGGAGCAAACAGGGGCTAAGTCCA |
| | | C |
| 1028 | 5996_Human_quadMut_8C_35A_45T_48T_n1 | GGGGGAGCCTGCTGGTGAATATTAACCAAGGTCAACCCAGTTATTGGTGGAGCAAACAGGGGCTAAGTCCA |
| | | C |

TABLE 11
Single or adjacent di-nucleotide substitution variants of Chinese
Tree Shrew SERPINA1 enhancer with higher luciferase expression
than original sequence SEQ ID NO: 122

| SEQ | | |
|---|---|---|
| ID | Chinese Tree Shrew | |
| NO: | SerpEnh variant | Sequence |
| 1029 | 2290_ChineseTreeShrewMod_ | GAAGGCTGTTGGTGAATATTAACCAAGGTC |
| | monoMut_G2A_n1 | ACCTCAGTTATCGGAGGAGCAAACAAGGGC |
| | | TAAGTCCAC |
| 1030 | 2293_ChineseTreeShrewMod_ | GGCGGCTGTTGGTGAATATTAACCAAGGTC |
| | monoMut_A3C_n1 | ACCTCAGTTATCGGAGGAGCAAACAAGGGC |
| | | TAAGTCCAC |
| 1031 | 2295_ChineseTreeShrewMod_ | GGTGGCTGTTGGTGAATATTAACCAAGGTC |
| | monoMut_A3T_n1 | ACCTCAGTTATCGGAGGAGCAAACAAGGGC |
| | | TAAGTCCAC |
| 1032 | 2296_ChineseTreeShrewMod_ | GGAAGCTGTTGGTGAATATTAACCAAGGTC |
| | monoMut_G4A_n1 | ACCTCAGTTATCGGAGGAGCAAACAAGGGC |
| | | TAAGTCCAC |
| 1033 | 2298_ChineseTreeShrewMod_ | GGATGCTGTTGGTGAATATTAACCAAGGTC |
| | monoMut_G4T_n1 | ACCTCAGTTATCGGAGGAGCAAACAAGGGC |
| | | TAAGTCCAC |
| 1034 | 2302_ChineseTreeShrewMod_ | GGAGGATGTTGGTGAATATTAACCAAGGTC |
| | monoMut_C6A_n1 | ACCTCAGTTATCGGAGGAGCAAACAAGGGC |
| | | TAAGTCCAC |
| 1035 | 2305_ChineseTreeShrewMod_ | GGAGGCAGTTGGTGAATATTAACCAAGGTC |
| | monoMut_T7A_n1 | ACCTCAGTTATCGGAGGAGCAAACAAGGGC |
| | | TAAGTCCAC |
| 1036 | 2309_ChineseTreeShrewMod_ | GGAGGCTCTTGGTGAATATTAACCAAGGTC |
| | monoMut_G8C_n1 | ACCTCAGTTATCGGAGGAGCAAACAAGGGC |
| | | TAAGTCCAC |
| 1037 | 2310_ChineseTreeShrewMod_ | GGAGGCTTTTGGTGAATATTAACCAAGGTC |
| | monoMut_G8T_n1 | ACCTCAGTTATCGGAGGAGCAAACAAGGGC |
| | | TAAGTCCAC |
| 1038 | 2314_ChineseTreeShrewMod_ | GGAGGCTGTAGGTGAATATTAACCAAGGTC |
| | monoMut_T10A_n1 | ACCTCAGTTATCGGAGGAGCAAACAAGGGC |
| | | TAAGTCCAC |
| 1039 | 2316_ChineseTreeShrewMod_ | GGAGGCTGTGGGTGAATATTAACCAAGGTC |
| | monoMut_T10G_n1 | ACCTCAGTTATCGGAGGAGCAAACAAGGGC |
| | | TAAGTCCAC |
| 1040 | 2317_ChineseTreeShrewMod_ | GGAGGCTGTTAGTGAATATTAACCAAGGTC |
| | monoMut_G11A_n1 | ACCTCAGTTATCGGAGGAGCAAACAAGGGC |
| | | TAAGTCCAC |
| 1041 | 2319_ChineseTreeShrewMod_ | GGAGGCTGTTTGTGAATATTAACCAAGGTC |
| | monoMut_G11T_n1 | ACCTCAGTTATCGGAGGAGCAAACAAGGGC |
| | | TAAGTCCAC |
| 1042 | 2321_ChineseTreeShrewMod_ | GGAGGCTGTTGCTGAATATTAACCAAGGTC |
| | monoMut_G12C_n1 | ACCTCAGTTATCGGAGGAGCAAACAAGGGC |
| | | TAAGTCCAC |
| 1043 | 2325_ChineseTreeShrewMod_ | GGAGGCTGTTGGGGAATATTAACCAAGGTC |
| | monoMut_T13G_n1 | ACCTCAGTTATCGGAGGAGCAAACAAGGGC |
| | | TAAGTCCAC |
| 1044 | 2326_ChineseTreeShrewMod_ | GGAGGCTGTTGGTAAATATTAACCAAGGTC |
| | monoMut_G14A_n1 | ACCTCAGTTATCGGAGGAGCAAACAAGGGC |
| | | TAAGTCCAC |
| 1045 | 2327_ChineseTreeShrewMod_ | GGAGGCTGTTGGTCAATATTAACCAAGGTC |
| | monoMut_G14C_n1 | ACCTCAGTTATCGGAGGAGCAAACAAGGGC |
| | | TAAGTCCAC |
| 1046 | 2328_ChineseTreeShrewMod_ | GGAGGCTGTTGGTTAATATTAACCAAGGTC |
| | monoMut_G14T_n1 | ACCTCAGTTATCGGAGGAGCAAACAAGGGC |
| | | TAAGTCCAC |
| 1047 | 2331_ChineseTreeShrewMod_ | GGAGGCTGTTGGTGTATATTAACCAAGGTC |
| | monoMut_A15T_n1 | ACCTCAGTTATCGGAGGAGCAAACAAGGGC |
| | | TAAGTCCAC |
| 1048 | 2332_ChineseTreeShrewMod_ | GGAGGCTGTTGGTGACTATTAACCAAGGTC |
| | monoMut_A16C_n1 | ACCTCAGTTATCGGAGGAGCAAACAAGGGC |
| | | TAAGTCCAC |
| 1049 | 2334_ChineseTreeShrewMod_ | GGAGGCTGTTGGTGATTATTAACCAAGGTC |
| | monoMut_A16T_n1 | ACCTCAGTTATCGGAGGAGCAAACAAGGGC |
| | | TAAGTCCAC |
| 1050 | 2336_ChineseTreeShrewMod_ | GGAGGCTGTTGGTGAACATTAACCAAGGTC |
| | monoMut_T17C_n1 | ACCTCAGTTATCGGAGGAGCAAACAAGGGC |
| | | TAAGTCCAC |
| 1051 | 2337_ChineseTreeShrewMod_ | GGAGGCTGTTGGTGAAGATTAACCAAGGTC |
| | monoMut_T17G_n1 | ACCTCAGTTATCGGAGGAGCAAACAAGGGC |
| | | TAAGTCCAC |
| 1052 | 2344_ChineseTreeShrewMod_ | GGAGGCTGTTGGTGAATATAAACCAAGGTC |
| | monoMut_T20A_n1 | ACCTCAGTTATCGGAGGAGCAAACAAGGGC |
| | | TAAGTCCAC |
| 1053 | 2345_ChineseTreeShrewMod_ | GGAGGCTGTTGGTGAATATCAACCAAGGTC |
| | monoMut_T20C_n1 | ACCTCAGTTATCGGAGGAGCAAACAAGGGC |
| | | TAAGTCCAC |
| 1054 | 2346_ChineseTreeShrewMod_ | GGAGGCTGTTGGTGAATATGAACCAAGGTC |
| | monoMut_T20G_n1 | ACCTCAGTTATCGGAGGAGCAAACAAGGGC |
| | | TAAGTCCAC |
| 1055 | 2351_ChineseTreeShrewMod_ | GGAGGCTGTTGGTGAATATTAGCCAAGGTC |
| | monoMut_A22G_n1 | ACCTCAGTTATCGGAGGAGCAAACAAGGGC |
| | | TAAGTCCAC |
| 1056 | 2352_ChineseTreeShrewMod_ | GGAGGCTGTTGGTGAATATTATCCAAGGTC |
| | monoMut_A22T_n1 | ACCTCAGTTATCGGAGGAGCAAACAAGGGC |
| | | TAAGTCCAC |
| 1057 | 2360_ChineseTreeShrewMod_ | GGAGGCTGTTGGTGAATATTAACCGAGGTC |
| | monoMut_A25G_n1 | ACCTCAGTTATCGGAGGAGCAAACAAGGGC |
| | | TAAGTCCAC |
| 1058 | 2363_ChineseTreeShrewMod_ | GGAGGCTGTTGGTGAATATTAACCAGGGTC |
| | monoMut_A26G_n1 | ACCTCAGTTATCGGAGGAGCAAACAAGGGC |
| | | TAAGTCCAC |
| 1059 | 2365_ChineseTreeShrewMod_ | GGAGGCTGTTGGTGAATATTAACCAAAGTC |
| | monoMut_G27A_n1 | ACCTCAGTTATCGGAGGAGCAAACAAGGGC |
| | | TAAGTCCAC |
| 1060 | 2369_ChineseTreeShrewMod_ | GGAGGCTGTTGGTGAATATTAACCAAGCTC |
| | monoMut_G28C_n1 | ACCTCAGTTATCGGAGGAGCAAACAAGGGC |
| | | TAAGTCCAC |
| 1061 | 2372_ChineseTreeShrewMod_ | GGAGGCTGTTGGTGAATATTAACCAAGGCC |
| | monoMut_T29C_n1 | ACCTCAGTTATCGGAGGAGCAAACAAGGGC |
| | | TAAGTCCAC |
| 1062 | 2373_ChineseTreeShrewMod_ | GGAGGCTGTTGGTGAATATTAACCAAGGGC |
| | monoMut_T29G_n1 | ACCTCAGTTATCGGAGGAGCAAACAAGGGC |
| | | TAAGTCCAC |
| 1063 | 2375_ChineseTreeShrewMod_ | GGAGGCTGTTGGTGAATATTAACCAAGGTG |
| | monoMut_C30G_n1 | ACCTCAGTTATCGGAGGAGCAAACAAGGGC |
| | | TAAGTCCAC |
| 1064 | 2378_ChineseTreeShrewMod_ | GGAGGCTGTTGGTGAATATTAACCAAGGTC |
| | monoMut_A31G_n1 | GCCTCAGTTATCGGAGGAGCAAACAAGGGC |
| | | TAAGTCCAC |
| 1065 | 2379_ChineseTreeShrewMod_ | GGAGGCTGTTGGTGAATATTAACCAAGGTC |
| | monoMut_A31T_n1 | TCCTCAGTTATCGGAGGAGCAAACAAGGGC |
| | | TAAGTCCAC |
| 1066 | 2380_ChineseTreeShrewMod_ | GGAGGCTGTTGGTGAATATTAACCAAGGTC |
| | monoMut_C32A_n1 | AACTCAGTTATCGGAGGAGCAAACAAGGGC |
| | | TAAGTCCAC |
| 1067 | 2381_ChineseTreeShrewMod_ | GGAGGCTGTTGGTGAATATTAACCAAGGTC |
| | monoMut_C32G_n1 | AGCTCAGTTATCGGAGGAGCAAACAAGGGC |
| | | TAAGTCCAC |
| 1068 | 2382_ChineseTreeShrewMod_ | GGAGGCTGTTGGTGAATATTAACCAAGGTC |
| | monoMut_C32T_n1 | ATCTCAGTTATCGGAGGAGCAAACAAGGGC |
| | | TAAGTCCAC |
| 1069 | 2383_ChineseTreeShrewMod_ | GGAGGCTGTTGGTGAATATTAACCAAGGTC |
| | monoMut_C33A_n1 | ACATCAGTTATCGGAGGAGCAAACAAGGGC |
| | | TAAGTCCAC |
| 1070 | 2385_ChineseTreeShrewMod_ | GGAGGCTGTTGGTGAATATTAACCAAGGTC |
| | monoMut_C33T_n1 | ACTTCAGTTATCGGAGGAGCAAACAAGGGC |
| | | TAAGTCCAC |
| 1071 | 2389_ChineseTreeShrewMod_ | GGAGGCTGTTGGTGAATATTAACCAAGGTC |
| | monoMut_C35A_n1 | ACCTAAGTTATCGGAGGAGCAAACAAGGGC |
| | | TAAGTCCAC |
| 1072 | 2390_ChineseTreeShrewMod_ | GGAGGCTGTTGGTGAATATTAACCAAGGTC |
| | monoMut_C35G_n1 | ACCTGAGTTATCGGAGGAGCAAACAAGGGC |
| | | TAAGTCCAC |
| 1073 | 2392_ChineseTreeShrewMod_ | GGAGGCTGTTGGTGAATATTAACCAAGGTC |
| | monoMut_A36C_n1 | ACCTCCGTTATCGGAGGAGCAAACAAGGGC |
| | | TAAGTCCAC |
| 1072 | 2393_ChineseTreeShrewMod_ | GGAGGCTGTTGGTGAATATTAACCAAGGTC |
| | monoMut_A36G_n1 | ACCTCGGTTATCGGAGGAGCAAACAAGGGC |
| | | TAAGTCCAC |
| 1075 | 2394_ChineseTreeShrewMod_ | GGAGGCTGTTGGTGAATATTAACCAAGGTC |
| | monoMut_A36T_n1 | ACCTCTGTTATCGGAGGAGCAAACAAGGGC |
| | | TAAGTCCAC |
| 1076 | 2396_ChineseTreeShrewMod_ | GGAGGCTGTTGGTGAATATTAACCAAGGTC |
| | monoMut_G37C_n1 | ACCTCACTTATCGGAGGAGCAAACAAGGGC |
| | | TAAGTCCAC |
| 1077 | 2398_ChineseTreeShrewMod_ | GGAGGCTGTTGGTGAATATTAACCAAGGTC |
| | monoMut_T38A_n1 | ACCTCAGATATCGGAGGAGCAAACAAGGGC |
| | | TAAGTCCAC |
| 1078 | 2399_ChineseTreeShrewMod_ | GGAGGCTGTTGGTGAATATTAACCAAGGTC |
| | monoMut_T38C_n1 | ACCTCAGCTATCGGAGGAGCAAACAAGGGC |
| | | TAAGTCCAC |
| 1079 | 2400_ChineseTreeShrewMod_ | GGAGGCTGTTGGTGAATATTAACCAAGGTC |
| | monoMut_T38G_n1 | ACCTCAGGTATCGGAGGAGCAAACAAGGGC |
| | | TAAGTCCAC |
| 1080 | 2402_ChineseTreeShrewMod_ | GGAGGCTGTTGGTGAATATTAACCAAGGTC |
| | monoMut_T39C_n1 | ACCTCAGTCATCGGAGGAGCAAACAAGGGC |
| | | TAAGTCCAC |
| 1081 | 2403_ChineseTreeShrewMod_ | GGAGGCTGTTGGTGAATATTAACCAAGGTC |
| | monoMut_T39G_n1 | ACCTCAGTGATCGGAGGAGCAAACAAGGGC |
| | | TAAGTCCAC |
| 1082 | 2405_ChineseTreeShrewMod_ | GGAGGCTGTTGGTGAATATTAACCAAGGTC |
| | monoMut_A40G_n1 | ACCTCAGTTGTCGGAGGAGCAAACAAGGGC |
| | | TAAGTCCAC |
| 1083 | 2407_ChineseTreeShrewMod_ | GGAGGCTGTTGGTGAATATTAACCAAGGTC |
| | monoMut_T41A_n1 | ACCTCAGTTAACGGAGGAGCAAACAAGGGC |
| | | TAAGTCCAC |
| 1084 | 2409_ChineseTreeShrewMod_ | GGAGGCTGTTGGTGAATATTAACCAAGGTC |
| | monoMut_T41G_n1 | ACCTCAGTTAGCGGAGGAGCAAACAAGGGC |
| | | TAAGTCCAC |
| 1085 | 2411_ChineseTreeShrewMod_ | GGAGGCTGTTGGTGAATATTAACCAAGGTC |
| | monoMut_C42G_n1 | ACCTCAGTTATGGGAGGAGCAAACAAGGGC |
| | | TAAGTCCAC |
| 1086 | 2413_ChineseTreeShrewMod_ | GGAGGCTGTTGGTGAATATTAACCAAGGTC |
| | monoMut_G43A_n1 | ACCTCAGTTATCAGAGGAGCAAACAAGGGC |
| | | TAAGTCCAC |
| 1087 | 2414_ChineseTreeShrewMod_ | GGAGGCTGTTGGTGAATATTAACCAAGGTC |
| | monoMut_G43C_n1 | ACCTCAGTTATCCGAGGAGCAAACAAGGGC |
| | | TAAGTCCAC |
| 1088 | 2416_ChineseTreeShrewMod_ | GGAGGCTGTTGGTGAATATTAACCAAGGTC |
| | monoMut_G44A_n1 | ACCTCAGTTATCGAAGGAGCAAACAAGGGC |
| | | TAAGTCCAC |
| 1089 | 2417_ChineseTreeShrewMod_ | GGAGGCTGTTGGTGAATATTAACCAAGGTC |
| | monoMut_G44C_n1 | ACCTCAGTTATCGCAGGAGCAAACAAGGGC |
| | | TAAGTCCAC |
| 1090 | 2419_ChineseTreeShrewMod_ | GGAGGCTGTTGGTGAATATTAACCAAGGTC |
| | monoMut_A45C_n1 | ACCTCAGTTATCGGCGGAGCAAACAAGGGC |
| | | TAAGTCCAC |
| 1091 | 2422_ChineseTreeShrewMod_ | GGAGGCTGTTGGTGAATATTAACCAAGGTC |
| | monoMut_G46A_n1 | ACCTCAGTTATCGGAAGAGCAAACAAGGGC |
| | | TAAGTCCAC |
| 1092 | 2423_ChineseTreeShrewMod_ | GGAGGCTGTTGGTGAATATTAACCAAGGTC |
| | monoMut_G46C_n1 | ACCTCAGTTATCGGACGAGCAAACAAGGGC |
| | | TAAGTCCAC |
| 1093 | 2424_ChineseTreeShrewMod_ | GGAGGCTGTTGGTGAATATTAACCAAGGTC |
| | monoMut_G46T_n1 | ACCTCAGTTATCGGATGAGCAAACAAGGGC |
| | | TAAGTCCAC |
| 1094 | 2425_ChineseTreeShrewMod_ | GGAGGCTGTTGGTGAATATTAACCAAGGTC |
| | monoMut_G47A_n1 | ACCTCAGTTATCGGAGAAGCAAACAAGGGC |
| | | TAAGTCCAC |
| 1095 | 2426_ChineseTreeShrewMod_ | GGAGGCTGTTGGTGAATATTAACCAAGGTC |
| | monoMut_G47C_n1 | ACCTCAGTTATCGGAGCAGCAAACAAGGGC |
| | | TAAGTCCAC |
| 1096 | 2428_ChineseTreeShrewMod_ | GGAGGCTGTTGGTGAATATTAACCAAGGTC |
| | monoMut_A48C_n1 | ACCTCAGTTATCGGAGGCGCAAACAAGGGC |
| | | TAAGTCCAC |
| 1097 | 2429_ChineseTreeShrewMod_ | GGAGGCTGTTGGTGAATATTAACCAAGGTC |
| | monoMut_A48G_n1 | ACCTCAGTTATCGGAGGGGCAAACAAGGGC |
| | | TAAGTCCAC |
| 1098 | 2430_ChineseTreeShrewMod_ | GGAGGCTGTTGGTGAATATTAACCAAGGTC |
| | monoMut_A48T_n1 | ACCTCAGTTATCGGAGGTGCAAACAAGGGC |
| | | TAAGTCCAC |
| 1099 | 2431_ChineseTreeShrewMod_ | GGAGGCTGTTGGTGAATATTAACCAAGGTC |
| | monoMut_G49A_n1 | ACCTCAGTTATCGGAGGAACAAACAAGGGC |
| | | TAAGTCCAC |
| 1100 | 2433_ChineseTreeShrewMod_ | GGAGGCTGTTGGTGAATATTAACCAAGGTC |
| | monoMut_G49T_n1 | ACCTCAGTTATCGGAGGATCAAACAAGGGC |
| | | TAAGTCCAC |
| 1101 | 2436_ChineseTreeShrewMod_ | GGAGGCTGTTGGTGAATATTAACCAAGGTC |
| | monoMut_C50T_n1 | ACCTCAGTTATCGGAGGAGTAAACAAGGGC |
| | | TAAGTCCAC |
| 1102 | 2437_ChineseTreeShrewMod_ | GGAGGCTGTTGGTGAATATTAACCAAGGTC |
| | monoMut_A51C_n1 | ACCTCAGTTATCGGAGGAGCCAACAAGGGC |
| | | TAAGTCCAC |
| 1103 | 2448_ChineseTreeShrewMod_ | GGAGGCTGTTGGTGAATATTAACCAAGGTC |
| | monoMut_C54T_n1 | ACCTCAGTTATCGGAGGAGCAAATAAGGGC |
| | | TAAGTCCAC |
| 1104 | 2450_ChineseTreeShrewMod_ | GGAGGCTGTTGGTGAATATTAACCAAGGTC |
| | monoMut_A55G_n1 | ACCTCAGTTATCGGAGGAGCAAACGAGGGC |
| | | TAAGTCCAC |
| 1105 | 2453_ChineseTreeShrewMod_ | GGAGGCTGTTGGTGAATATTAACCAAGGTC |
| | monoMut_A56G_n1 | ACCTCAGTTATCGGAGGAGCAAACAGGGGC |
| | | TAAGTCCAC |
| 1106 | 2454_ChineseTreeShrewMod_ | GGAGGCTGTTGGTGAATATTAACCAAGGTC |
| | monoMut_A56T_n1 | ACCTCAGTTATCGGAGGAGCAAACATGGGC |
| | | TAAGTCCAC |
| 1107 | 2457_ChineseTreeShrewMod_ | GGAGGCTGTTGGTGAATATTAACCAAGGTC |
| | monoMut_G57T_n1 | ACCTCAGTTATCGGAGGAGCAAACAATGGC |
| | | TAAGTCCAC |
| 1108 | 2459_ChineseTreeShrewMod_ | GGAGGCTGTTGGTGAATATTAACCAAGGTC |
| | monoMut_G58C_n1 | ACCTCAGTTATCGGAGGAGCAAACAAGCGC |
| | | TAAGTCCAC |
| 1109 | 2460_ChineseTreeShrewMod_ | GGAGGCTGTTGGTGAATATTAACCAAGGTC |
| | monoMut_G58T_n1 | ACCTCAGTTATCGGAGGAGCAAACAAGTGC |
| | | TAAGTCCAC |
| 1110 | 2461_ChineseTreeShrewMod_ | GGAGGCTGTTGGTGAATATTAACCAAGGTC |
| | monoMut_G59A_n1 | ACCTCAGTTATCGGAGGAGCAAACAAGGAC |
| | | TAAGTCCAC |
| 1111 | 2462_ChineseTreeShrewMod_ | GGAGGCTGTTGGTGAATATTAACCAAGGTC |
| | monoMut_G59C_n1 | ACCTCAGTTATCGGAGGAGCAAACAAGGCC |
| | | TAAGTCCAC |
| 1112 | 2463_ChineseTreeShrewMod_ | GGAGGCTGTTGGTGAATATTAACCAAGGTC |
| | monoMut_G59T_n1 | ACCTCAGTTATCGGAGGAGCAAACAAGGTC |
| | | TAAGTCCAC |
| 1113 | 2467_ChineseTreeShrewMod_ | GGAGGCTGTTGGTGAATATTAACCAAGGTC |
| | monoMut_T61A_n1 | ACCTCAGTTATCGGAGGAGCAAACAAGGGC |
| | | AAAGTCCAC |
| 1114 | 2469_ChineseTreeShrewMod_ | GGAGGCTGTTGGTGAATATTAACCAAGGTC |
| | monoMut_T61G_n1 | ACCTCAGTTATCGGAGGAGCAAACAAGGGC |
| | | GAAGTCCAC |
| 1115 | 2482_ChineseTreeShrewMod_ | GGAGGCTGTTGGTGAATATTAACCAAGGTC |
| | monoMut_C66A_n1 | ACCTCAGTTATCGGAGGAGCAAACAAGGGC |
| | | TAAGTACAC |
| 1116 | 2484_ChineseTreeShrewMod_ | GGAGGCTGTTGGTGAATATTAACCAAGGTC |
| | monoMut_C66T_n1 | ACCTCAGTTATCGGAGGAGCAAACAAGGGC |
| | | TAAGTTCAC |
| 1117 | 2491_ChineseTreeShrewMod_ | GGAGGCTGTTGGTGAATATTAACCAAGGTC |
| | monoMut_C69A_n1 | ACCTCAGTTATCGGAGGAGCAAACAAGGGC |
| | | TAAGTCCAA |
| 1118 | 2498_ChineseTreeShrewMod_ | CCAGGCTGTTGGTGAATATTAACCAAGGTC |
| | diMut_GG1CC_n1 | ACCTCAGTTATCGGAGGAGCAAACAAGGGC |
| | | TAAGTCCAC |
| 1119 | 2499_ChineseTreeShrewMod_ | CTAGGCTGTTGGTGAATATTAACCAAGGTC |
| | diMut_GG1CT_n1 | ACCTCAGTTATCGGAGGAGCAAACAAGGGC |
| | | TAAGTCCAC |
| 1110 | 2503_ChineseTreeShrewMod_ | GACGGCTGTTGGTGAATATTAACCAAGGTC |
| | diMut_GA2AC_n1 | ACCTCAGTTATCGGAGGAGCAAACAAGGGC |
| | | TAAGTCCAC |
| 1121 | 2504_ChineseTreeShrewMod_ | GAGGGCTGTTGGTGAATATTAACCAAGGTC |
| | diMut_GA2AG_n1 | ACCTCAGTTATCGGAGGAGCAAACAAGGGC |
| | | TAAGTCCAC |
| 1122 | 2505_ChineseTreeShrewMod_ | GATGGCTGTTGGTGAATATTAACCAAGGTC |
| | diMut_GA2AT_n1 | ACCTCAGTTATCGGAGGAGCAAACAAGGGC |
| | | TAAGTCCAC |
| 1123 | 2507_ChineseTreeShrewMod_ | GCGGGCTGTTGGTGAATATTAACCAAGGTC |
| | diMut_GA2CG_n1 | ACCTCAGTTATCGGAGGAGCAAACAAGGGC |
| | | TAAGTCCAC |
| 1124 | 2508_ChineseTreeShrewMod_ | GCTGGCTGTTGGTGAATATTAACCAAGGTC |
| | diMut_GA2CT_n1 | ACCTCAGTTATCGGAGGAGCAAACAAGGGC |
| | | TAAGTCCAC |
| 1125 | 2510_ChineseTreeShrewMod_ | GTGGGCTGTTGGTGAATATTAACCAAGGTC |
| | diMut_GA2TG_n1 | ACCTCAGTTATCGGAGGAGCAAACAAGGGC |
| | | TAAGTCCAC |
| 1126 | 2511_ChineseTreeShrewMod_ | GTTGGCTGTTGGTGAATATTAACCAAGGTC |
| | diMut_GA2TT_n1 | ACCTCAGTTATCGGAGGAGCAAACAAGGGC |
| | | TAAGTCCAC |
| 1127 | 2512_ChineseTreeShrewMod_ | GGCAGCTGTTGGTGAATATTAACCAAGGTC |
| | diMut_AG3CA_n1 | ACCTCAGTTATCGGAGGAGCAAACAAGGGC |
| | | TAAGTCCAC |
| 1128 | 2514_ChineseTreeShrewMod_ | GGCTGCTGTTGGTGAATATTAACCAAGGTC |
| | diMut_AG3CT_n1 | ACCTCAGTTATCGGAGGAGCAAACAAGGGC |
| | | TAAGTCCAC |
| 1129 | 2516_ChineseTreeShrewMod_ | GGGCGCTGTTGGTGAATATTAACCAAGGTC |
| | diMut_AG3GC_n1 | ACCTCAGTTATCGGAGGAGCAAACAAGGGC |
| | | TAAGTCCAC |
| 1130 | 2517_ChineseTreeShrewMod_ | GGGTGCTGTTGGTGAATATTAACCAAGGTC |
| | diMut_AG3GT_n1 | ACCTCAGTTATCGGAGGAGCAAACAAGGGC |
| | | TAAGTCCAC |
| 1131 | 2519_ChineseTreeShrewMod_ | GGTCGCTGTTGGTGAATATTAACCAAGGTC |
| | diMut_AG3TC_n1 | ACCTCAGTTATCGGAGGAGCAAACAAGGGC |
| | | TAAGTCCAC |
| 1132 | 2520_ChineseTreeShrewMod_ | GGTTGCTGTTGGTGAATATTAACCAAGGTC |
| | diMut_AG3TT_n1 | ACCTCAGTTATCGGAGGAGCAAACAAGGGC |
| | | TAAGTCCAC |
| 1133 | 2521_ChineseTreeShrewMod_ | GGAAACTGTTGGTGAATATTAACCAAGGTC |
| | diMut_GG4AA_n1 | ACCTCAGTTATCGGAGGAGCAAACAAGGGC |
| | | TAAGTCCAC |
| 1134 | 2523_ChineseTreeShrewMod_ | GGAATCTGTTGGTGAATATTAACCAAGGTC |
| | diMut_GG4AT_n1 | ACCTCAGTTATCGGAGGAGCAAACAAGGGC |
| | | TAAGTCCAC |
| 1135 | 2524_ChineseTreeShrewMod_ | GGACACTGTTGGTGAATATTAACCAAGGTC |
| | diMut_GG4CA_n1 | ACCTCAGTTATCGGAGGAGCAAACAAGGGC |
| | | TAAGTCCAC |
| 1136 | 2525_ChineseTreeShrewMod_ | GGACCCTGTTGGTGAATATTAACCAAGGTC |
| | diMut_GG4CC_n1 | ACCTCAGTTATCGGAGGAGCAAACAAGGGC |
| | | TAAGTCCAC |
| 1137 | 2526_ChineseTreeShrewMod_ | GGACTCTGTTGGTGAATATTAACCAAGGTC |
| | diMut_GG4CT_n1 | ACCTCAGTTATCGGAGGAGCAAACAAGGGC |
| | | TAAGTCCAC |
| 1138 | 2527_ChineseTreeShrewMod_ | GGATACTGTTGGTGAATATTAACCAAGGTC |
| | diMut_GG4TA_n1 | ACCTCAGTTATCGGAGGAGCAAACAAGGGC |
| | | TAAGTCCAC |
| 1139 | 2529_ChineseTreeShrewMod_ | GGATTCTGTTGGTGAATATTAACCAAGGTC |
| | diMut_GG4TT_n1 | ACCTCAGTTATCGGAGGAGCAAACAAGGGC |
| | | TAAGTCCAC |
| 1140 | 2530_ChineseTreeShrewMod_ | GGAGAATGTTGGTGAATATTAACCAAGGTC |
| | diMut_GC5AA_n1 | ACCTCAGTTATCGGAGGAGCAAACAAGGGC |
| | | TAAGTCCAC |
| 1141 | 2531_ChineseTreeShrewMod_ | GGAGAGTGTTGGTGAATATTAACCAAGGTC |
| | diMut_GC5AG_n1 | ACCTCAGTTATCGGAGGAGCAAACAAGGGC |
| | | TAAGTCCAC |
| 1142 | 2533_ChineseTreeShrewMod_ | GGAGCATGTTGGTGAATATTAACCAAGGTC |
| | diMut_GC5CA_n1 | ACCTCAGTTATCGGAGGAGCAAACAAGGGC |
| | | TAAGTCCAC |
| 1143 | 2534_ChineseTreeShrewMod_ | GGAGCGTGTTGGTGAATATTAACCAAGGTC |
| | diMut_GC5CG_n1 | ACCTCAGTTATCGGAGGAGCAAACAAGGGC |
| | | TAAGTCCAC |
| 1144 | 2536_ChineseTreeShrewMod_ | GGAGTATGTTGGTGAATATTAACCAAGGTC |
| | diMut_GC5TA_n1 | ACCTCAGTTATCGGAGGAGCAAACAAGGGC |
| | | TAAGTCCAC |
| 1145 | 2538_ChineseTreeShrewMod_ | GGAGTTTGTTGGTGAATATTAACCAAGGTC |
| | diMut_GC5TT_n1 | ACCTCAGTTATCGGAGGAGCAAACAAGGGC |
| | | TAAGTCCAC |
| 1146 | 2540_ChineseTreeShrewMod_ | GGAGGACGTTGGTGAATATTAACCAAGGTC |
| | diMut_CT6AC_n1 | ACCTCAGTTATCGGAGGAGCAAACAAGGGC |
| | | TAAGTCCAC |
| 1147 | 2541_ChineseTreeShrewMod_ | GGAGGAGGTTGGTGAATATTAACCAAGGTC |
| | diMut_CT6AG_n1 | ACCTCAGTTATCGGAGGAGCAAACAAGGGC |
| | | TAAGTCCAC |
| 1148 | 2544_ChineseTreeShrewMod_ | GGAGGGGGTTGGTGAATATTAACCAAGGTC |
| | diMut_CT6GG_n1 | ACCTCAGTTATCGGAGGAGCAAACAAGGGC |
| | | TAAGTCCAC |
| 1149 | 2547_ChineseTreeShrewMod_ | GGAGGTGGTTGGTGAATATTAACCAAGGTC |
| | diMut_CT6TG_n1 | ACCTCAGTTATCGGAGGAGCAAACAAGGGC |
| | | TAAGTCCAC |
| 1150 | 2548_ChineseTreeShrewMod_ | GGAGGCAATTGGTGAATATTAACCAAGGTC |
| | diMut_TG7AA_n1 | ACCTCAGTTATCGGAGGAGCAAACAAGGGC |
| | | TAAGTCCAC |
| 1151 | 2550_ChineseTreeShrewMod_ | GGAGGCATTTGGTGAATATTAACCAAGGTC |
| | diMut_TG7AT_n1 | ACCTCAGTTATCGGAGGAGCAAACAAGGGC |
| | | TAAGTCCAC |
| 1152 | 2552_ChineseTreeShrewMod_ | GGAGGCCCTTGGTGAATATTAACCAAGGTC |
| | diMut_TG7CC_n1 | ACCTCAGTTATCGGAGGAGCAAACAAGGGC |
| | | TAAGTCCAC |
| 1153 | 2553_ChineseTreeShrewMod_ | GGAGGCCTTTGGTGAATATTAACCAAGGTC |
| | diMut_TG7CT_n1 | ACCTCAGTTATCGGAGGAGCAAACAAGGGC |
| | | TAAGTCCAC |
| 1154 | 2554_ChineseTreeShrewMod_ | GGAGGCGATTGGTGAATATTAACCAAGGTC |
| | diMut_TG7GA_n1 | ACCTCAGTTATCGGAGGAGCAAACAAGGGC |
| | | TAAGTCCAC |
| 1155 | 2555_ChineseTreeShrewMod_ | GGAGGCGCTTGGTGAATATTAACCAAGGTC |
| | diMut_TG7GC_n1 | ACCTCAGTTATCGGAGGAGCAAACAAGGGC |
| | | TAAGTCCAC |
| 1156 | 2556_ChineseTreeShrewMod_ | GGAGGCGTTTGGTGAATATTAACCAAGGTC |
| | diMut_TG7GT_n1 | ACCTCAGTTATCGGAGGAGCAAACAAGGGC |
| | | TAAGTCCAC |
| 1157 | 2557_ChineseTreeShrewMod_ | GGAGGCTAATGGTGAATATTAACCAAGGTC |
| | diMut_GT8AA_n1 | ACCTCAGTTATCGGAGGAGCAAACAAGGGC |
| | | TAAGTCCAC |
| 1158 | 2558_ChineseTreeShrewMod_ | GGAGGCTACTGGTGAATATTAACCAAGGTC |
| | diMut_GT8AC_n1 | ACCTCAGTTATCGGAGGAGCAAACAAGGGC |
| | | TAAGTCCAC |
| 1159 | 2559_ChineseTreeShrewMod_ | GGAGGCTAGTGGTGAATATTAACCAAGGTC |
| | diMut_GT8AG_n1 | ACCTCAGTTATCGGAGGAGCAAACAAGGGC |
| | | TAAGTCCAC |
| 1160 | 2560_ChineseTreeShrewMod_ | GGAGGCTCATGGTGAATATTAACCAAGGTC |
| | diMut_GT8CA_n1 | ACCTCAGTTATCGGAGGAGCAAACAAGGGC |
| | | TAAGTCCAC |
| 1161 | 2561_ChineseTreeShrewMod_ | GGAGGCTCCTGGTGAATATTAACCAAGGTC |
| | diMut_GT8CC_n1 | ACCTCAGTTATCGGAGGAGCAAACAAGGGC |
| | | TAAGTCCAC |
| 1162 | 2563_ChineseTreeShrewMod_ | GGAGGCTTATGGTGAATATTAACCAAGGTC |
| | diMut_GT8TA_n1 | ACCTCAGTTATCGGAGGAGCAAACAAGGGC |
| | | TAAGTCCAC |
| 1163 | 2564_ChineseTreeShrewMod_ | GGAGGCTTCTGGTGAATATTAACCAAGGTC |
| | diMut_GT8TC_n1 | ACCTCAGTTATCGGAGGAGCAAACAAGGGC |
| | | TAAGTCCAC |
| 1164 | 2566_ChineseTreeShrewMod_ | GGAGGCTGAAGGTGAATATTAACCAAGGTC |
| | diMut_TT9AA_n1 | ACCTCAGTTATCGGAGGAGCAAACAAGGGC |
| | | TAAGTCCAC |
| 1165 | 2567_ChineseTreeShrewMod_ | GGAGGCTGACGGTGAATATTAACCAAGGTC |
| | diMut_TT9AC_n1 | ACCTCAGTTATCGGAGGAGCAAACAAGGGC |
| | | TAAGTCCAC |
| 1166 | 2568_ChineseTreeShrewMod_ | GGAGGCTGAGGGTGAATATTAACCAAGGTC |
| | diMut_TT9AG_n1 | ACCTCAGTTATCGGAGGAGCAAACAAGGGC |
| | | TAAGTCCAC |
| 1167 | 2574_ChineseTreeShrewMod_ | GGAGGCTGGGGGTGAATATTAACCAAGGTC |
| | diMut_TT9GG_n1 | ACCTCAGTTATCGGAGGAGCAAACAAGGGC |
| | | TAAGTCCAC |
| 1168 | 2575_ChineseTreeShrewMod_ | GGAGGCTGTAAGTGAATATTAACCAAGGTC |
| | diMut_TG10AA_n1 | ACCTCAGTTATCGGAGGAGCAAACAAGGGC |
| | | TAAGTCCAC |
| 1169 | 2580_ChineseTreeShrewMod_ | GGAGGCTGTCTGTGAATATTAACCAAGGTC |
| | diMut_TG10CT_n1 | ACCTCAGTTATCGGAGGAGCAAACAAGGGC |
| | | TAAGTCCAC |
| 1170 | 2582_ChineseTreeShrewMod_ | GGAGGCTGTGCGTGAATATTAACCAAGGTC |
| | diMut_TG10GC_n1 | ACCTCAGTTATCGGAGGAGCAAACAAGGGC |
| | | TAAGTCCAC |
| 1171 | 2586_ChineseTreeShrewMod_ | GGAGGCTGTTATTGAATATTAACCAAGGTC |
| | diMut_GG11AT_n1 | ACCTCAGTTATCGGAGGAGCAAACAAGGGC |
| | | TAAGTCCAC |
| 1172 | 2591_ChineseTreeShrewMod_ | GGAGGCTGTTTCTGAATATTAACCAAGGTC |
| | diMut_GG11TC_n1 | ACCTCAGTTATCGGAGGAGCAAACAAGGGC |
| | | TAAGTCCAC |
| 1173 | 2596_ChineseTreeShrewMod_ | GGAGGCTGTTGCAGAATATTAACCAAGGTC |
| | diMut_GT12CA_n1 | ACCTCAGTTATCGGAGGAGCAAACAAGGGC |
| | | TAAGTCCAC |
| 1174 | 2597_ChineseTreeShrewMod_ | GGAGGCTGTTGCCGAATATTAACCAAGGTC |
| | diMut_GT12CC_n1 | ACCTCAGTTATCGGAGGAGCAAACAAGGGC |
| | | TAAGTCCAC |
| 1175 | 2599_ChineseTreeShrewMod_ | GGAGGCTGTTGTAGAATATTAACCAAGGTC |
| | diMut_GT12TA_n1 | ACCTCAGTTATCGGAGGAGCAAACAAGGGC |
| | | TAAGTCCAC |
| 1176 | 2600_ChineseTreeShrewMod_ | GGAGGCTGTTGTCGAATATTAACCAAGGTC |
| | diMut_GT12TC_n1 | ACCTCAGTTATCGGAGGAGCAAACAAGGGC |
| | | TAAGTCCAC |
| 1177 | 2601_ChineseTreeShrewMod_ | GGAGGCTGTTGTGGAATATTAACCAAGGTC |
| | diMut_GT12TG_n1 | ACCTCAGTTATCGGAGGAGCAAACAAGGGC |
| | | TAAGTCCAC |
| 1178 | 2602_ChineseTreeShrewMod_ | GGAGGCTGTTGGAAAATATTAACCAAGGTC |
| | diMut_TG13AA_n1 | ACCTCAGTTATCGGAGGAGCAAACAAGGGC |
| | | TAAGTCCAC |
| 1179 | 2603_ChineseTreeShrewMod_ | GGAGGCTGTTGGACAATATTAACCAAGGTC |
| | diMut_TG13AC_n1 | ACCTCAGTTATCGGAGGAGCAAACAAGGGC |
| | | TAAGTCCAC |
| 1180 | 2605_ChineseTreeShrewMod_ | GGAGGCTGTTGGCAAATATTAACCAAGGTC |
| | diMut_TG13CA_n1 | ACCTCAGTTATCGGAGGAGCAAACAAGGGC |
| | | TAAGTCCAC |
| 1181 | 2606_ChineseTreeShrewMod_ | GGAGGCTGTTGGCCAATATTAACCAAGGTC |
| | diMut_TG13CC_n1 | ACCTCAGTTATCGGAGGAGCAAACAAGGGC |
| | | TAAGTCCAC |
| 1183 | 2608_ChineseTreeShrewMod_ | GGAGGCTGTTGGGAAATATTAACCAAGGTC |
| | diMut_TG13GA_n1 | ACCTCAGTTATCGGAGGAGCAAACAAGGGC |
| | | TAAGTCCAC |
| 1183 | 2609_ChineseTreeShrewMod_ | GGAGGCTGTTGGGCAATATTAACCAAGGTC |
| | diMut_TG13GC_n1 | ACCTCAGTTATCGGAGGAGCAAACAAGGGC |
| | | TAAGTCCAC |
| 1184 | 2610_ChineseTreeShrewMod_ | GGAGGCTGTTGGGTAATATTAACCAAGGTC |
| | diMut_TG13GT_n1 | ACCTCAGTTATCGGAGGAGCAAACAAGGGC |
| | | TAAGTCCAC |
| 1185 | 2611_ChineseTreeShrewMod_ | GGAGGCTGTTGGTACATATTAACCAAGGTC |
| | diMut_GA14AC_n1 | ACCTCAGTTATCGGAGGAGCAAACAAGGGC |
| | | TAAGTCCAC |
| 1186 | 2614_ChineseTreeShrewMod_ | GGAGGCTGTTGGTCCATATTAACCAAGGTC |
| | diMut_GA14CC_n1 | ACCTCAGTTATCGGAGGAGCAAACAAGGGC |
| | | TAAGTCCAC |
| 1187 | 2615_ChineseTreeShrewMod_ | GGAGGCTGTTGGTCGATATTAACCAAGGTC |
| | diMut_GA14CG_n1 | ACCTCAGTTATCGGAGGAGCAAACAAGGGC |
| | | TAAGTCCAC |
| 1188 | 2623_ChineseTreeShrewMod_ | GGAGGCTGTTGGTGGCTATTAACCAAGGTC |
| | diMut_AA15GC_n1 | ACCTCAGTTATCGGAGGAGCAAACAAGGGC |
| | | TAAGTCCAC |
| 1189 | 2626_ChineseTreeShrewMod_ | GGAGGCTGTTGGTGTCTATTAACCAAGGTC |
| | diMut_AA15TC_n1 | ACCTCAGTTATCGGAGGAGCAAACAAGGGC |
| | | TAAGTCCAC |
| 1190 | 2628_ChineseTreeShrewMod_ | GGAGGCTGTTGGTGTTTATTAACCAAGGTC |
| | diMut_AA15TT_n1 | ACCTCAGTTATCGGAGGAGCAAACAAGGGC |
| | | TAAGTCCAC |
| 1191 | 2629_ChineseTreeShrewMod_ | GGAGGCTGTTGGTGACAATTAACCAAGGTC |
| | diMut_AT16CA_n1 | ACCTCAGTTATCGGAGGAGCAAACAAGGGC |
| | | TAAGTCCAC |
| 1192 | 2633_ChineseTreeShrewMod_ | GGAGGCTGTTGGTGAGCATTAACCAAGGTC |
| | diMut_AT16GC_n1 | ACCTCAGTTATCGGAGGAGCAAACAAGGGC |
| | | TAAGTCCAC |
| 1193 | 2635_ChineseTreeShrewMod_ | GGAGGCTGTTGGTGATAATTAACCAAGGTC |
| | diMut_AT16TA_n1 | ACCTCAGTTATCGGAGGAGCAAACAAGGGC |
| | | TAAGTCCAC |
| 1194 | 2636_ChineseTreeShrewMod_ | GGAGGCTGTTGGTGATCATTAACCAAGGTC |
| | diMut_AT16TC_n1 | ACCTCAGTTATCGGAGGAGCAAACAAGGGC |
| | | TAAGTCCAC |
| 1195 | 2640_ChineseTreeShrewMod_ | GGAGGCTGTTGGTGAAATTTAACCAAGGTC |
| | diMut_TA17AT_n1 | ACCTCAGTTATCGGAGGAGCAAACAAGGGC |
| | | TAAGTCCAC |
| 1196 | 2643_ChineseTreeShrewMod_ | GGAGGCTGTTGGTGAACTTTAACCAAGGTC |
| | diMut_TA17CT_n1 | ACCTCAGTTATCGGAGGAGCAAACAAGGGC |
| | | TAAGTCCAC |
| 1197 | 2644_ChineseTreeShrewMod_ | GGAGGCTGTTGGTGAAGCTTAACCAAGGTC |
| | diMut_TA17GC_n1 | ACCTCAGTTATCGGAGGAGCAAACAAGGGC |
| | | TAAGTCCAC |
| 1198 | 2646_ChineseTreeShrewMod_ | GGAGGCTGTTGGTGAAGTTTAACCAAGGTC |
| | diMut_TA17GT_n1 | ACCTCAGTTATCGGAGGAGCAAACAAGGGC |
| | | TAAGTCCAC |
| 1199 | 2647_ChineseTreeShrewMod_ | GGAGGCTGTTGGTGAATCATAACCAAGGTC |
| | diMut_AT18CA_n1 | ACCTCAGTTATCGGAGGAGCAAACAAGGGC |
| | | TAAGTCCAC |
| 1200 | 2651_ChineseTreeShrewMod_ | GGAGGCTGTTGGTGAATGCTAACCAAGGTC |
| | diMut_AT18GC_n1 | ACCTCAGTTATCGGAGGAGCAAACAAGGGC |
| | | TAAGTCCAC |
| 1201 | 2653_ChineseTreeShrewMod_ | GGAGGCTGTTGGTGAATTATAACCAAGGTC |
| | diMut_AT18TA_n1 | ACCTCAGTTATCGGAGGAGCAAACAAGGGC |
| | | TAAGTCCAC |
| 1202 | 2654_ChineseTreeShrewMod_ | GGAGGCTGTTGGTGAATTCTAACCAAGGTC |
| | diMut_AT18TC_n1 | ACCTCAGTTATCGGAGGAGCAAACAAGGGC |
| | | TAAGTCCAC |
| 1203 | 2664_ChineseTreeShrewMod_ | GGAGGCTGTTGGTGAATAGGAACCAAGGTC |
| | diMut_TT19GG_n1 | ACCTCAGTTATCGGAGGAGCAAACAAGGGC |
| | | TAAGTCCAC |
| 1204 | 2672_ChineseTreeShrewMod_ | GGAGGCTGTTGGTGAATATGGACCAAGGTC |
| | diMut_TA20GG_n1 | ACCTCAGTTATCGGAGGAGCAAACAAGGGC |
| | | TAAGTCCAC |
| 1205 | 2678_ChineseTreeShrewMod_ | GGAGGCTGTTGGTGAATATTGGCCAAGGTC |
| | diMut_AA21GG_n1 | ACCTCAGTTATCGGAGGAGCAAACAAGGGC |
| | | TAAGTCCAC |
| 1206 | 2679_ChineseTreeShrewMod_ | GGAGGCTGTTGGTGAATATTGTCCAAGGTC |
| | diMut_AA21GT_n1 | ACCTCAGTTATCGGAGGAGCAAACAAGGGC |
| | | TAAGTCCAC |
| 1207 | 2685_ChineseTreeShrewMod_ | GGAGGCTGTTGGTGAATATTACTCAAGGTC |
| | diMut_AC22CT_n1 | ACCTCAGTTATCGGAGGAGCAAACAAGGGC |
| | | TAAGTCCAC |
| 1208 | 2707_ChineseTreeShrewMod_ | GGAGGCTGTTGGTGAATATTAACTCAGGTC |
| | diMut_CA24TC_n1 | ACCTCAGTTATCGGAGGAGCAAACAAGGGC |
| | | TAAGTCCAC |
| 1209 | 2714_ChineseTreeShrewMod_ | GGAGGCTGTTGGTGAATATTAACCGGGGTC |
| | diMut_AA25GG_n1 | ACCTCAGTTATCGGAGGAGCAAACAAGGGC |
| | | TAAGTCCAC |
| 1210 | 2717_ChineseTreeShrewMod_ | GGAGGCTGTTGGTGAATATTAACCTGGGTC |
| | diMut_AA25TG_n1 | ACCTCAGTTATCGGAGGAGCAAACAAGGGC |
| | | TAAGTCCAC |
| 1211 | 2728_ChineseTreeShrewMod_ | GGAGGCTGTTGGTGAATATTAACCAAAATC |
| | diMut_GG27AA_n1 | ACCTCAGTTATCGGAGGAGCAAACAAGGGC |
| | | TAAGTCCAC |
| 1212 | 2734_ChineseTreeShrewMod_ | GGAGGCTGTTGGTGAATATTAACCAATATC |
| | diMut_GG27TA_n1 | ACCTCAGTTATCGGAGGAGCAAACAAGGGC |
| | | TAAGTCCAC |
| 1213 | 2743_ChineseTreeShrewMod_ | GGAGGCTGTTGGTGAATATTAACCAAGTAC |
| | diMut_GT28TA_n1 | ACCTCAGTTATCGGAGGAGCAAACAAGGGC |
| | | TAAGTCCAC |
| 1214 | 2748_ChineseTreeShrewMod_ | GGAGGCTGTTGGTGAATATTAACCAAGGAT |
| | diMut_TC29AT_n1 | ACCTCAGTTATCGGAGGAGCAAACAAGGGC |
| | | TAAGTCCAC |
| 1215 | 2750_ChineseTreeShrewMod_ | GGAGGCTGTTGGTGAATATTAACCAAGGCG |
| | diMut_TC29CG_n1 | ACCTCAGTTATCGGAGGAGCAAACAAGGGC |
| | | TAAGTCCAC |
| 1216 | 2753_ChineseTreeShrewMod_ | GGAGGCTGTTGGTGAATATTAACCAAGGGG |
| | diMut_TC29GG_n1 | ACCTCAGTTATCGGAGGAGCAAACAAGGGC |
| | | TAAGTCCAC |
| 1217 | 2754_ChineseTreeShrewMod_ | GGAGGCTGTTGGTGAATATTAACCAAGGGT |
| | diMut_TC29GT_n1 | ACCTCAGTTATCGGAGGAGCAAACAAGGGC |
| | | TAAGTCCAC |
| 1218 | 2756_ChineseTreeShrewMod_ | GGAGGCTGTTGGTGAATATTAACCAAGGTA |
| | diMut_CA30AG_n1 | GCCTCAGTTATCGGAGGAGCAAACAAGGGC |
| | | TAAGTCCAC |
| 1219 | 2758_ChineseTreeShrewMod_ | GGAGGCTGTTGGTGAATATTAACCAAGGTG |
| | diMut_CA30GC_n1 | CCCTCAGTTATCGGAGGAGCAAACAAGGGC |
| | | TAAGTCCAC |
| 1220 | 2759_ChineseTreeShrewMod_ | GGAGGCTGTTGGTGAATATTAACCAAGGTG |
| | diMut_CA30GG_n1 | GCCTCAGTTATCGGAGGAGCAAACAAGGGC |
| | | TAAGTCCAC |
| 1221 | 2763_ChineseTreeShrewMod_ | GGAGGCTGTTGGTGAATATTAACCAAGGTT |
| | diMut_CA30TT_n1 | TCCTCAGTTATCGGAGGAGCAAACAAGGGC |
| | | TAAGTCCAC |
| 1222 | 2768_ChineseTreeShrewMod_ | GGAGGCTGTTGGTGAATATTAACCAAGGTC |
| | diMut_AC31GG_n1 | GGCTCAGTTATCGGAGGAGCAAACAAGGGC |
| | | TAAGTCCAC |
| 1223 | 2769_ChineseTreeShrewMod_ | GGAGGCTGTTGGTGAATATTAACCAAGGTC |
| | diMut_AC31GT_n1 | GTCTCAGTTATCGGAGGAGCAAACAAGGGC |
| | | TAAGTCCAC |
| 1224 | 2770_ChineseTreeShrewMod_ | GGAGGCTGTTGGTGAATATTAACCAAGGTC |
| | diMut_AC31TA_n1 | TACTCAGTTATCGGAGGAGCAAACAAGGGC |
| | | TAAGTCCAC |
| 1225 | 2771_ChineseTreeShrewMod_ | GGAGGCTGTTGGTGAATATTAACCAAGGTC |
| | diMut_AC31TG_n1 | TGCTCAGTTATCGGAGGAGCAAACAAGGGC |
| | | TAAGTCCAC |
| 1226 | 2773_ChineseTreeShrewMod_ | GGAGGCTGTTGGTGAATATTAACCAAGGTC |
| | diMut_CC32AA_n1 | AAATCAGTTATCGGAGGAGCAAACAAGGGC |
| | | TAAGTCCAC |
| 1227 | 2774_ChineseTreeShrewMod_ | GGAGGCTGTTGGTGAATATTAACCAAGGTC |
| | diMut_CC32AG_n1 | AAGTCAGTTATCGGAGGAGCAAACAAGGGC |
| | | TAAGTCCAC |
| 1228 | 2778_ChineseTreeShrewMod_ | GGAGGCTGTTGGTGAATATTAACCAAGGIC |
| | diMut_CC32GT_n1 | AGTTCAGITATCGGAGGAGCAAACAAGGGC |
| | | TAAGTCCAC |
| 1229 | 2779_ChineseTreeShrewMod_ | GGAGGCTGTTGGTGAATATTAACCAAGGTC |
| | diMut_CC32TA_n1 | ATATCAGTTATCGGAGGAGCAAACAAGGGC |
| | | TAAGTCCAC |
| 1230 | 2780_ChineseTreeShrewMod_ | GGAGGCTGTTGGTGAATATTAACCAAGGTC |
| | diMut_CC32TG_n1 | ATGTCAGTTATCGGAGGAGCAAACAAGGGC |
| | | TAAGTCCAC |
| 1231 | 2781_ChineseTreeShrewMod_ | GGAGGCTGTTGGTGAATATTAACCAAGGTC |
| | diMut_CC32TT_n1 | ATTTCAGTTATCGGAGGAGCAAACAAGGGC |
| | | TAAGTCCAC |
| 1232 | 2782_ChineseTreeShrewMod_ | GGAGGCTGTTGGTGAATATTAACCAAGGTC |
| | diMut_CT33AA_n1 | ACAACAGTTATCGGAGGAGCAAACAAGGGC |
| | | TAAGTCCAC |
| 1233 | 2783_ChineseTreeShrewMod_ | GGAGGCTGTTGGTGAATATTAACCAAGGTC |
| | diMut_CT33AC_n1 | ACACCAGTTATCGGAGGAGCAAACAAGGGC |
| | | TAAGTCCAC |
| 1234 | 2784_ChineseTreeShrewMod_ | GGAGGCTGTTGGTGAATATTAACCAAGGTC |
| | diMut_CT33AG_n1 | ACAGCAGTTATCGGAGGAGCAAACAAGGGC |
| | | TAAGTCCAC |
| 1235 | 2785_ChineseTreeShrewMod_ | GGAGGCTGTTGGTGAATATTAACCAAGGTC |
| | diMut_CT33GA_n1 | ACGACAGTTATCGGAGGAGCAAACAAGGGC |
| | | TAAGTCCAC |
| 1236 | 2786_ChineseTreeShrewMod_ | GGAGGCTGTTGGTGAATATTAACCAAGGTC |
| | diMut_CT33GC_n1 | ACGCCAGTTATCGGAGGAGCAAACAAGGGC |
| | | TAAGTCCAC |
| 1237 | 2789_ChineseTreeShrewMod_ | GGAGGCTGTTGGTGAATATTAACCAAGGTC |
| | diMut_CT33TC_n1 | ACTCCAGTTATCGGAGGAGCAAACAAGGGC |
| | | TAAGTCCAC |
| 1238 | 2791_ChineseTreeShrewMod_ | GGAGGCTGTTGGTGAATATTAACCAAGGTC |
| | diMut_TC34AA_n1 | ACCAAAGTTATCGGAGGAGCAAACAAGGGC |
| | | TAAGTCCAC |
| 1239 | 2792_ChineseTreeShrewMod_ | GGAGGCTGTTGGTGAATATTAACCAAGGTC |
| | diMut_TC34AG_n1 | ACCAGAGTTATCGGAGGAGCAAACAAGGGC |
| | | TAAGTCCAC |
| 1240 | 2793_ChineseTreeShrewMod_ | GGAGGCTGTTGGTGAATATTAACCAAGGTC |
| | diMut_TC34AT_n1 | ACCATAGTTATCGGAGGAGCAAACAAGGGC |
| | | TAAGTCCAC |
| 1241 | 2798_ChineseTreeShrewMod_ | GGAGGCTGTTGGTGAATATTAACCAAGGTC |
| | diMut_TC34GG_n1 | ACCGGAGTTATCGGAGGAGCAAACAAGGGC |
| | | TAAGTCCAC |
| 1242 | 2799_ChineseTreeShrewMod_ | GGAGGCTGTTGGTGAATATTAACCAAGGTC |
| | diMut_TC34GT_n1 | ACCGTAGTTATCGGAGGAGCAAACAAGGGC |
| | | TAAGTCCAC |
| 1243 | 2800_ChineseTreeShrewMod_ | GGAGGCTGTTGGTGAATATTAACCAAGGTC |
| | diMut_CA35AC_n1 | ACCTACGTTATCGGAGGAGCAAACAAGGGC |
| | | TAAGTCCAC |
| 1244 | 2801_ChineseTreeShrewMod_ | GGAGGCTGTTGGTGAATATTAACCAAGGTC |
| | diMut_CA35AG_n1 | ACCTAGGTTATCGGAGGAGCAAACAAGGGC |
| | | TAAGTCCAC |
| 1245 | 2802_ChineseTreeShrewMod_ | GGAGGCTGTTGGTGAATATTAACCAAGGTC |
| | diMut_CA35AT_n1 | ACCTATGTTATCGGAGGAGCAAACAAGGGC |
| | | TAAGTCCAC |
| 1246 | 2803_ChineseTreeShrewMod_ | GGAGGCTGTTGGTGAATATTAACCAAGGTC |
| | diMut_CA35GC_n1 | ACCTGCGTTATCGGAGGAGCAAACAAGGGC |
| | | TAAGTCCAC |
| 1247 | 2804_ChineseTreeShrewMod_ | GGAGGCTGTTGGTGAATATTAACCAAGGTC |
| | diMut_CA35GG_n1 | ACCTGGGTTATCGGAGGAGCAAACAAGGGC |
| | | TAAGTCCAC |
| 1248 | 2808_ChineseTreeShrewMod_ | GGAGGCTGTTGGTGAATATTAACCAAGGTC |
| | diMut_CA35TT_n1 | ACCTTTGTTATCGGAGGAGCAAACAAGGGC |
| | | TAAGTCCAC |
| 1249 | 2810_ChineseTreeShrewMod_ | GGAGGCTGTTGGTGAATATTAACCAAGGTC |
| | diMut_AG36CC_n1 | ACCTCCCTTATCGGAGGAGCAAACAAGGGC |
| | | TAAGTCCAC |
| 1250 | 2811_ChineseTreeShrewMod_ | GGAGGCTGTTGGTGAATATTAACCAAGGTC |
| | diMut_AG36CT_n1 | ACCTCCTTTATCGGAGGAGCAAACAAGGGC |
| | | TAAGTCCAC |
| 1251 | 2813_ChineseTreeShrewMod_ | GGAGGCTGTTGGTGAATATTAACCAAGGTC |
| | diMut_AG36GC_n1 | ACCTCGCTTATCGGAGGAGCAAACAAGGGC |
| | | TAAGTCCAC |
| 1252 | 2814_ChineseTreeShrewMod_ | GGAGGCTGTTGGTGAATATTAACCAAGGTC |
| | diMut_AG36GT_n1 | ACCTCGTTTATCGGAGGAGCAAACAAGGGC |
| | | TAAGTCCAC |
| 1253 | 2815_ChineseTreeShrewMod_ | GGAGGCTGTTGGTGAATATTAACCAAGGTC |
| | diMut_AG36TA_n1 | ACCTCTATTATCGGAGGAGCAAACAAGGGC |
| | | TAAGTCCAC |
| 1254 | 2816_ChineseTreeShrewMod_ | GGAGGCTGTTGGTGAATATTAACCAAGGTC |
| | diMut_AG36TC_n1 | ACCTCTCTTATCGGAGGAGCAAACAAGGGC |
| | | TAAGTCCAC |
| 1255 | 2820_ChineseTreeShrewMod_ | GGAGGCTGTTGGTGAATATTAACCAAGGTC |
| | diMut_GT37AG_n1 | ACCTCAAGTATCGGAGGAGCAAACAAGGGC |
| | | TAAGTCCAC |
| 1256 | 2821_ChineseTreeShrewMod_ | GGAGGCTGTTGGTGAATATTAACCAAGGTC |
| | diMut_GT37CA_n1 | ACCTCACATATCGGAGGAGCAAACAAGGGC |
| | | TAAGTCCAC |
| 1257 | 2825_ChineseTreeShrewMod_ | GGAGGCTGTTGGTGAATATTAACCAAGGTC |
| | diMut_GT37TC_n1 | ACCTCATCTATCGGAGGAGCAAACAAGGGC |
| | | TAAGTCCAC |
| 1258 | 2831_ChineseTreeShrewMod_ | GGAGGCTGTTGGTGAATATTAACCAAGGTC |
| | diMut_TT38CC_n1 | ACCTCAGCCATCGGAGGAGCAAACAAGGGC |
| | | TAAGTCCAC |
| 1259 | 2832_ChineseTreeShrewMod_ | GGAGGCTGTTGGTGAATATTAACCAAGGTC |
| | diMut_TT38CG_n1 | ACCTCAGCGATCGGAGGAGCAAACAAGGGC |
| | | TAAGTCCAC |
| 1260 | 2833_ChineseTreeShrewMod_ | GGAGGCTGTTGGTGAATATTAACCAAGGTC |
| | diMut_TT38GA_n1 | ACCTCAGGAATCGGAGGAGCAAACAAGGGC |
| | | TAAGTCCAC |
| 1261 | 2834_ChineseTreeShrewMod_ | GGAGGCTGTTGGTGAATATTAACCAAGGTC |
| | diMut_TT38GC_n1 | ACCTCAGGCATCGGAGGAGCAAACAAGGGC |
| | | TAAGTCCAC |
| 1262 | 2836_ChineseTreeShrewMod_ | GGAGGCTGTTGGTGAATATTAACCAAGGTC |
| | diMut_TA39AC_n1 | ACCTCAGTACTCGGAGGAGCAAACAAGGGC |
| | | TAAGTCCAC |
| 1263 | 2837_ChineseTreeShrewMod_ | GGAGGCTGTTGGTGAATATTAACCAAGGTC |
| | diMut_TA39AG_n1 | ACCTCAGTAGTCGGAGGAGCAAACAAGGGC |
| | | TAAGTCCAC |
| 1264 | 2838_ChineseTreeShrewMod_ | GGAGGCTGTTGGTGAATATTAACCAAGGTC |
| | diMut_TA39AT_n1 | ACCTCAGTATTCGGAGGAGCAAACAAGGGC |
| | | TAAGTCCAC |
| 1265 | 2843_ChineseTreeShrewMod_ | GGAGGCTGTTGGTGAATATTAACCAAGGTC |
| | diMut_TA39GG_n1 | ACCTCAGTGGTCGGAGGAGCAAACAAGGGC |
| | | TAAGTCCAC |
| 1266 | 2846_ChineseTreeShrewMod_ | GGAGGCTGTTGGTGAATATTAACCAAGGTC |
| | diMut_AT40CC_n1 | ACCTCAGTTCCCGGAGGAGCAAACAAGGGC |
| | | TAAGTCCAC |
| 1267 | 2847_ChineseTreeShrewMod_ | GGAGGCTGTTGGTGAATATTAACCAAGGTC |
| | diMut_AT40CG_n1 | ACCTCAGTTCGCGGAGGAGCAAACAAGGGC |
| | | TAAGTCCAC |
| 1268 | 2848_ChineseTreeShrewMod_ | GGAGGCTGTTGGTGAATATTAACCAAGGTC |
| | diMut_AT40GA_n1 | ACCTCAGTTGACGGAGGAGCAAACAAGGGC |
| | | TAAGTCCAC |
| 1269 | 2849_ChineseTreeShrewMod_ | GGAGGCTGTTGGTGAATATTAACCAAGGTC |
| | diMut_AT40GC_n1 | ACCTCAGTTGCCGGAGGAGCAAACAAGGGC |
| | | TAAGTCCAC |
| 1270 | 2851_ChineseTreeShrewMod_ | GGAGGCTGTTGGTGAATATTAACCAAGGTC |
| | diMut_AT40TA_n1 | ACCTCAGTTTACGGAGGAGCAAACAAGGGC |
| | | TAAGTCCAC |
| 1271 | 2852_ChineseTreeShrewMod_ | GGAGGCTGTTGGTGAATATTAACCAAGGTC |
| | diMut_AT40TC_n1 | ACCTCAGTTTCCGGAGGAGCAAACAAGGGC |
| | | TAAGTCCAC |
| 1272 | 2855_ChineseTreeShrewMod_ | GGAGGCTGTTGGTGAATATTAACCAAGGTC |
| | diMut_TC41AG_n1 | ACCTCAGTTAAGGGAGGAGCAAACAAGGGC |
| | | TAAGTCCAC |
| 1273 | 2858_ChineseTreeShrewMod_ | GGAGGCTGTTGGTGAATATTAACCAAGGTC |
| | diMut_TC41CG_n1 | ACCTCAGTTACGGGAGGAGCAAACAAGGGC |
| | | TAAGTCCAC |
| 1274 | 2862_ChineseTreeShrewMod_ | GGAGGCTGTTGGTGAATATTAACCAAGGTC |
| | diMut_TC41GT_n1 | ACCTCAGTTAGTGGAGGAGCAAACAAGGGC |
| | | TAAGTCCAC |
| 1275 | 2864_ChineseTreeShrewMod_ | GGAGGCTGTTGGTGAATATTAACCAAGGTC |
| | diMut_CG42AC_n1 | ACCTCAGTTATACGAGGAGCAAACAAGGGC |
| | | TAAGTCCAC |
| 1276 | 2866_ChineseTreeShrewMod_ | GGAGGCTGTTGGTGAATATTAACCAAGGTC |
| | diMut_CG42GA_n1 | ACCTCAGTTATGAGAGGAGCAAACAAGGGC |
| | | TAAGTCCAC |
| 1277 | 2867_ChineseTreeShrewMod_ | GGAGGCTGTTGGTGAATATTAACCAAGGTC |
| | diMut_CG42GC_n1 | ACCTCAGTTATGCGAGGAGCAAACAAGGGC |
| | | TAAGTCCAC |
| 1278 | 2868_ChineseTreeShrewMod_ | GGAGGCTGTTGGTGAATATTAACCAAGGTC |
| | diMut_CG42GT_n1 | ACCTCAGTTATGTGAGGAGCAAACAAGGGC |
| | | TAAGTCCAC |
| 1279 | 2869_ChineseTreeShrewMod_ | GGAGGCTGTTGGTGAATATTAACCAAGGTC |
| | diMut_CG42TA_n1 | ACCTCAGTTATTAGAGGAGCAAACAAGGGC |
| | | TAAGTCCAC |
| 1280 | 2870_ChineseTreeShrewMod_ | GGAGGCTGTTGGTGAATATTAACCAAGGTC |
| | diMut_CG42TC_n1 | ACCTCAGTTATTCGAGGAGCAAACAAGGGC |
| | | TAAGTCCAC |
| 1281 | 2872_ChineseTreeShrewMod_ | GGAGGCTGTTGGTGAATATTAACCAAGGTC |
| | diMut_GG43AA_n1 | ACCTCAGTTATCAAAGGAGCAAACAAGGGC |
| | | TAAGTCCAC |
| 1282 | 2873_ChineseTreeShrewMod_ | GGAGGCTGTTGGTGAATATTAACCAAGGTC |
| | diMut_GG43AC_n1 | ACCTCAGTTATCACAGGAGCAAACAAGGGC |
| | | TAAGTCCAC |
| 1283 | 2874_ChineseTreeShrewMod_ | GGAGGCTGTTGGTGAATATTAACCAAGGTC |
| | diMut_GG43AT_n1 | ACCTCAGTTATCATAGGAGCAAACAAGGGC |
| | | TAAGTCCAC |
| 1284 | 2879_ChineseTreeShrewMod_ | GGAGGCTGTTGGTGAATATTAACCAAGGTC |
| | diMut_GG43TC_n1 | ACCTCAGTTATCTCAGGAGCAAACAAGGGC |
| | | TAAGTCCAC |
| 1285 | 2880_ChineseTreeShrewMod_ | GGAGGCTGTTGGTGAATATTAACCAAGGTC |
| | diMut_GG43TT_n1 | ACCTCAGTTATCTTAGGAGCAAACAAGGGC |
| | | TAAGTCCAC |
| 1286 | 2882_ChineseTreeShrewMod_ | GGAGGCTGTTGGTGAATATTAACCAAGGTC |
| | diMut_GA44AG_n1 | ACCTCAGTTATCGAGGGAGCAAACAAGGGC |
| | | TAAGTCCAC |
| 1287 | 2884_ChineseTreeShrewMod_ | GGAGGCTGTTGGTGAATATTAACCAAGGTC |
| | diMut_GA44CC_n1 | ACCTCAGTTATCGCCGGAGCAAACAAGGGC |
| | | TAAGTCCAC |
| 1288 | 2886_ChineseTreeShrewMod_ | GGAGGCTGTTGGTGAATATTAACCAAGGTC |
| | diMut_GA44CT_n1 | ACCTCAGTTATCGCTGGAGCAAACAAGGGC |
| | | TAAGTCCAC |
| 1289 | 2890_ChineseTreeShrewMod_ | GGAGGCTGTTGGTGAATATTAACCAAGGTC |
| | diMut_AG45CA_n1 | ACCTCAGTTATCGGCAGAGCAAACAAGGGC |
| | | TAAGTCCAC |
| 1290 | 2891_ChineseTreeShrewMod_ | GGAGGCTGTTGGTGAATATTAACCAAGGTC |
| | diMut_AG45CC_n1 | ACCTCAGTTATCGGCCGAGCAAACAAGGGC |
| | | TAAGTCCAC |
| 1291 | 2892_ChineseTreeShrewMod_ | GGAGGCTGTTGGTGAATATTAACCAAGGTC |
| | diMut_AG45CT_n1 | ACCTCAGTTATCGGCTGAGCAAACAAGGGC |
| | | TAAGTCCAC |
| 1292 | 2893_ChineseTreeShrewMod_ | GGAGGCTGTTGGTGAATATTAACCAAGGTC |
| | diMut_AG45GA_n1 | ACCTCAGTTATCGGGAGAGCAAACAAGGGC |
| | | TAAGTCCAC |
| 1293 | 2894_ChineseTreeShrewMod_ | GGAGGCTGTTGGTGAATATTAACCAAGGTC |
| | diMut_AG45GC_n1 | ACCTCAGTTATCGGGCGAGCAAACAAGGGC |
| | | TAAGTCCAC |
| 1294 | 2895_ChineseTreeShrewMod_ | GGAGGCTGTTGGTGAATATTAACCAAGGTC |
| | diMut_AG45GT_n1 | ACCTCAGTTATCGGGTGAGCAAACAAGGGC |
| | | TAAGTCCAC |
| 1295 | 2896_ChineseTreeShrewMod_ | GGAGGCTGTTGGTGAATATTAACCAAGGTC |
| | diMut_AG45TA_n1 | ACCTCAGTTATCGGTAGAGCAAACAAGGGC |
| | | TAAGTCCAC |
| 1296 | 2897_ChineseTreeShrewMod_ | GGAGGCTGTTGGTGAATATTAACCAAGGTC |
| | diMut_AG45TC_n1 | ACCTCAGTTATCGGTCGAGCAAACAAGGGC |
| | | TAAGTCCAC |
| 1297 | 2898_ChineseTreeShrewMod_ | GGAGGCTGTTGGTGAATATTAACCAAGGTC |
| | diMut_AG45TT_n1 | ACCTCAGTTATCGGTTGAGCAAACAAGGGC |
| | | TAAGTCCAC |
| 1298 | 2899_ChineseTreeShrewMod_ | GGAGGCTGTTGGTGAATATTAACCAAGGTC |
| | diMut_GG46AA_n1 | ACCTCAGTTATCGGAAAAGCAAACAAGGGC |
| | | TAAGTCCAC |
| 1299 | 2900_ChineseTreeShrewMod_ | GGAGGCTGTTGGTGAATATTAACCAAGGTC |
| | diMut_GG46AC_n1 | ACCTCAGTTATCGGAACAGCAAACAAGGGC |
| | | TAAGTCCAC |
| 1300 | 2901_ChineseTreeShrewMod_ | GGAGGCTGTTGGTGAATATTAACCAAGGTC |
| | diMut_GG46AT_n1 | ACCTCAGTTATCGGAATAGCAAACAAGGGC |
| | | TAAGTCCAC |
| 1301 | 2902_ChineseTreeShrewMod_ | GGAGGCTGTTGGTGAATATTAACCAAGGTC |
| | diMut_GG46CA_n1 | ACCTCAGTTATCGGACAAGCAAACAAGGGC |
| | | TAAGTCCAC |
| 1302 | 2903_ChineseTreeShrewMod_ | GGAGGCTGTTGGTGAATATTAACCAAGGTC |
| | diMut_GG46CC_n1 | ACCTCAGTTATCGGACCAGCAAACAAGGGC |
| | | TAAGTCCAC |
| 1303 | 2905_ChineseTreeShrewMod_ | GGAGGCTGTTGGTGAATATTAACCAAGGTC |
| | diMut_GG46TA_n1 | ACCTCAGTTATCGGATAAGCAAACAAGGGC |
| | | TAAGTCCAC |
| 1304 | 2906_ChineseTreeShrewMod_ | GGAGGCTGTTGGTGAATATTAACCAAGGTC |
| | diMut_GG46TC_n1 | ACCTCAGTTATCGGATCAGCAAACAAGGGC |
| | | TAAGTCCAC |
| 1305 | 2908_ChineseTreeShrewMod_ | GGAGGCTGTTGGTGAATATTAACCAAGGTC |
| | diMut_GA47AC_n1 | ACCTCAGTTATCGGAGACGCAAACAAGGGC |
| | | TAAGTCCAC |
| 1306 | 2909_ChineseTreeShrewMod_ | GGAGGCTGTTGGTGAATATTAACCAAGGTC |
| | diMut_GA47AG_n1 | ACCTCAGTTATCGGAGAGGCAAACAAGGGC |
| | | TAAGTCCAC |
| 1307 | 2911_ChineseTreeShrewMod_ | GGAGGCTGTTGGTGAATATTAACCAAGGTC |
| | diMut_GA47CC_n1 | ACCTCAGTTATCGGAGCCGCAAACAAGGGC |
| | | TAAGTCCAC |
| 1308 | 2912_ChineseTreeShrewMod_ | GGAGGCTGTTGGTGAATATTAACCAAGGTC |
| | diMut_GA47CG_n1 | ACCTCAGTTATCGGAGCGGCAAACAAGGGC |
| | | TAAGTCCAC |
| 1309 | 2915_ChineseTreeShrewMod_ | GGAGGCTGTTGGTGAATATTAACCAAGGTC |
| | diMut_GA47TG_n1 | ACCTCAGTTATCGGAGTGGCAAACAAGGGC |
| | | TAAGTCCAC |
| 1310 | 2917_ChineseTreeShrewMod_ | GGAGGCTGTTGGTGAATATTAACCAAGGTC |
| | diMut_AG48CA_n1 | ACCTCAGTTATCGGAGGCACAAACAAGGGC |
| | | TAAGTCCAC |
| 1311 | 2919_ChineseTreeShrewMod_ | GGAGGCTGTTGGTGAATATTAACCAAGGTC |
| | diMut_AG48CT_n1 | ACCTCAGTTATCGGAGGCTCAAACAAGGGC |
| | | TAAGTCCAC |
| 1312 | 2921_ChineseTreeShrewMod_ | GGAGGCTGTTGGTGAATATTAACCAAGGTC |
| | diMut_AG48GC_n1 | ACCTCAGTTATCGGAGGGCCAAACAAGGGC |
| | | TAAGTCCAC |
| 1313 | 2922_ChineseTreeShrewMod_ | GGAGGCTGTTGGTGAATATTAACCAAGGTC |
| | diMut_AG48GT_n1 | ACCTCAGTTATCGGAGGGTCAAACAAGGGC |
| | | TAAGTCCAC |
| 1314 | 2923_ChineseTreeShrewMod_ | GGAGGCTGTTGGTGAATATTAACCAAGGTC |
| | diMut_AG48TA_n1 | ACCTCAGTTATCGGAGGTACAAACAAGGGC |
| | | TAAGTCCAC |
| 1315 | 2924_ChineseTreeShrewMod_ | GGAGGCTGTTGGTGAATATTAACCAAGGTC |
| | diMut_AG48TC_n1 | ACCTCAGTTATCGGAGGTCCAAACAAGGGC |
| | | TAAGTCCAC |
| 1316 | 2928_ChineseTreeShrewMod_ | GGAGGCTGTTGGTGAATATTAACCAAGGTC |
| | diMut_GC49AT_n1 | ACCTCAGTTATCGGAGGAATAAACAAGGGC |
| | | TAAGTCCAC |
| 1317 | 2931_ChineseTreeShrewMod_ | GGAGGCTGTTGGTGAATATTAACCAAGGTC |
| | diMut_GC49CT_n1 | ACCTCAGTTATCGGAGGACTAAACAAGGGC |
| | | TAAGTCCAC |
| 1318 | 2934_ChineseTreeShrewMod_ | GGAGGCTGTTGGTGAATATTAACCAAGGTC |
| | diMut_GC49TT_n1 | ACCTCAGTTATCGGAGGATTAAACAAGGGC |
| | | TAAGTCCAC |
| 1319 | 2940_ChineseTreeShrewMod_ | GGAGGCTGTTGGTGAATATTAACCAAGGTC |
| | diMut_CA50GT_n1 | ACCTCAGTTATCGGAGGAGGTAACAAGGGC |
| | | TAAGTCCAC |
| 1320 | 2959_ChineseTreeShrewMod_ | GGAGGCTGTTGGTGAATATTAACCAAGGTC |
| | diMut_AA52TC_n1 | ACCTCAGTTATCGGAGGAGCATCCAAGGGC |
| | | TAAGTCCAC |
| 1321 | 2973_ChineseTreeShrewMod_ | GGAGGCTGTTGGTGAATATTAACCAAGGTC |
| | diMut_CA54AT_n1 | ACCTCAGTTATCGGAGGAGCAAAATAGGGC |
| | | TAAGTCCAC |
| 1322 | 2977_ChineseTreeShrewMod_ | GGAGGCTGTTGGTGAATATTAACCAAGGTC |
| | diMut_CA54TC_n1 | ACCTCAGTTATCGGAGGAGCAAATCAGGGC |
| | | TAAGTCCAC |
| 1323 | 2978_ChineseTreeShrewMod_ | GGAGGCTGTTGGTGAATATTAACCAAGGTC |
| | diMut_CA54TG_n1 | ACCTCAGTTATCGGAGGAGCAAATGAGGGC |
| | | TAAGTCCAC |
| 1324 | 2995_ChineseTreeShrewMod_ | GGAGGCTGTTGGTGAATATTAACCAAGGTC |
| | diMut_AG56TA_n1 | ACCTCAGTTATCGGAGGAGCAAACATAGGC |
| | | TAAGTCCAC |
| 1325 | 3013_ChineseTreeShrewMod_ | GGAGGCTGTTGGTGAATATTAACCAAGGTC |
| | diMut_GG58TA_n1 | ACCTCAGTTATCGGAGGAGCAAACAAGTAC |
| | | TAAGTCCAC |
| 1326 | 3014_ChineseTreeShrewMod_ | GGAGGCTGTTGGTGAATATTAACCAAGGTC |
| | diMut_GG58TC_n1 | ACCTCAGTTATCGGAGGAGCAAACAAGTCC |
| | | TAAGTCCAC |
| 1327 | 3015_ChineseTreeShrewMod_ | GGAGGCTGTTGGTGAATATTAACCAAGGTC |
| | diMut_GG58TT_n1 | ACCTCAGTTATCGGAGGAGCAAACAAGTTC |
| | | TAAGTCCAC |
| 1328 | 3025_ChineseTreeShrewMod_ | GGAGGCTGTTGGTGAATATTAACCAAGGTC |
| | diMut_CT60AA_n1 | ACCTCAGTTATCGGAGGAGCAAACAAGGGA |
| | | AAAGTCCAC |
| 1329 | 3028_ChineseTreeShrewMod_ | GGAGGCTGTTGGTGAATATTAACCAAGGTC |
| | diMut_CT60GA_n1 | ACCTCAGTTATCGGAGGAGCAAACAAGGGG |
| | | AAAGTCCAC |
| 1330 | 3031_ChineseTreeShrewMod_ | GGAGGCTGTTGGTGAATATTAACCAAGGTC |
| | diMut_CT60TA_n1 | ACCTCAGTTATCGGAGGAGCAAACAAGGGT |
| | | AAAGTCCAC |
| 1331 | 3036_ChineseTreeShrewMod_ | GGAGGCTGTTGGTGAATATTAACCAAGGTC |
| | diMut_TA61AT_n1 | ACCTCAGTTATCGGAGGAGCAAACAAGGGC |
| | | ATAGTCCAC |
| 1332 | 3073_ChineseTreeShrewMod_ | GGAGGCTGTTGGTGAATATTAACCAAGGTC |
| | diMut_TC65CA_n1 | ACCTCAGTTATCGGAGGAGCAAACAAGGGC |
| | | TAAGCACAC |
| 1333 | 3078_ChineseTreeShrewMod_ | GGAGGCTGTTGGTGAATATTAACCAAGGTC |
| | diMut_TC65GT_n1 | ACCTCAGTTATCGGAGGAGCAAACAAGGGC |
| | | TAAGGTCAC |
| 1334 | 3079_ChineseTreeShrewMod_ | GGAGGCTGTTGGTGAATATTAACCAAGGTC |
| | diMut_CC66AA_n1 | ACCTCAGTTATCGGAGGAGCAAACAAGGGC |
| | | TAAGTAAAC |
| 1335 | 3090_ChineseTreeShrewMod_ | GGAGGCTGTTGGTGAATATTAACCAAGGTC |
| | diMut_CA67AT_n1 | ACCTCAGTTATCGGAGGAGCAAACAAGGGC |
| | | TAAGTCATC |

TABLE 12
Single and adjacent di-nucleotide variants of BushBaby SERPINA1 enhancer with higher luciferase
expression than original sequence SEQ ID NO: 83

| SEQ ID NO: | Bushbaby SERPINA1 enhancer variant | Sequence |
|---|---|---|
| 1336 | 1256_Bushbaby_monoMut_G1A_n1 | AGGGGAAGCTACTGGTGAATATTAACCAAGGTCACCCAGTTATCAGGGAGCAAACAGGAGCTAAGTCCAT |
| 1337 | 1262_Bushbaby_monoMut_G3A_n1 | GGAGGAAGCTACTGGTGAATATTAACCAAGGTCACCCAGTTATCAGGGAGCAAACAGGAGCTAAGTCCAT |
| 1338 | 1263_Bushbaby_monoMut_G3C_n1 | GGCGGAAGCTACTGGTGAATATTAACCAAGGTCACCCAGTTATCAGGGAGCAAACAGGAGCTAAGTCCAT |
| 1339 | 1270_Bushbaby_monoMut_G5T_n1 | GGGGTAAGCTACTGGTGAATATTAACCAAGGTCACCCAGTTATCAGGGAGCAAACAGGAGCTAAGTCCAT |
| 1340 | 1273_Bushbaby_monoMut_A6T_n1 | GGGGGTAGCTACTGGTGAATATTAACCAAGGTCACCCAGTTATCAGGGAGCAAACAGGAGCTAAGTCCAT |
| 1341 | 1277_Bushbaby_monoMut_G8A_n1 | GGGGGAAACTACTGGTGAATATTAACCAAGGTCACCCAGTTATCAGGGAGCAAACAGGAGCTAAGTCCAT |
| 1342 | 1282_Bushbaby_monoMut_C9T_n1 | GGGGGAAGTTACTGGTGAATATTAACCAAGGTCACCCAGTTATCAGGGAGCAAACAGGAGCTAAGTCCAT |
| 1343 | 1284_Bushbaby_monoMut_T10C_n1 | GGGGGAAGCCACTGGTGAATATTAACCAAGGTCACCCAGTTATCAGGGAGCAAACAGGAGCTAAGTCCAT |
| 1344 | 1286_Bushbaby_monoMut_A11C_n1 | GGGGGAAGCTCCTGGTGAATATTAACCAAGGTCACCCAGTTATCAGGGAGCAAACAGGAGCTAAGTCCAT |
| 1345 | 1287_Bushbaby_monoMut_A11G_n1 | GGGGGAAGCTGCTGGTGAATATTAACCAAGGTCACCCAGTTATCAGGGAGCAAACAGGAGCTAAGTCCAT |
| 1346 | 1288_Bushbaby_monoMut_A11T_n1 | GGGGGAAGCTTCTGGTGAATATTAACCAAGGTCACCCAGTTATCAGGGAGCAAACAGGAGCTAAGTCCAT |
| 1347 | 1294_Bushbaby_monoMut_T13G_n1 | GGGGGAAGCTACGGGTGAATATTAACCAAGGTCACCCAGTTATCAGGGAGCAAACAGGAGCTAAGTCCAT |
| 1348 | 1300_Bushbaby_monoMut_G15T_n1 | GGGGGAAGCTACTGTTGAATATTAACCAAGGTCACCCAGTTATCAGGGAGCAAACAGGAGCTAAGTCCAT |
| 1349 | 1306_Bushbaby_monoMut_G17T_n1 | GGGGGAAGCTACTGGTTAATATTAACCAAGGTCACCCAGTTATCAGGGAGCAAACAGGAGCTAAGTCCAT |
| 1350 | 1310_Bushbaby_monoMut_A19C_n1 | GGGGGAAGCTACTGGTGACTATTAACCAAGGTCACCCAGTTATCAGGGAGCAAACAGGAGCTAAGTCCAT |
| 1351 | 1311_Bushbaby_monoMut_A19G_n1 | GGGGGAAGCTACTGGTGAGTATTAACCAAGGTCACCCAGTTATCAGGGAGCAAACAGGAGCTAAGTCCAT |
| 1352 | 1324_Bushbaby_monoMut_T23G_n1 | GGGGGAAGCTACTGGTGAATATGAACCAAGGTCACCCAGTTATCAGGGAGCAAACAGGAGCTAAGTCCAT |
| 1353 | 1330_Bushbaby_monoMut_A25T_n1 | GGGGGAAGCTACTGGTGAATATTATCCAAGGTCACCCAGTTATCAGGGAGCAAACAGGAGCTAAGTCCAT |
| 1354 | 1352_Bushbaby_monoMut_C33A_n1 | GGGGGAAGCTACTGGTGAATATTAACCAAGGTAACCCAGTTATCAGGGAGCAAACAGGAGCTAAGTCCAT |
| 1355 | 1359_Bushbaby_monoMut_C35G_n1 | GGGGGAAGCTACTGGTGAATATTAACCAAGGTCAGCCAGTTATCAGGGAGCAAACAGGAGCTAAGTCCAT |
| 1356 | 1360_Bushbaby_monoMut_C35T_n1 | GGGGGAAGCTACTGGTGAATATTAACCAAGGTCATCCAGTTATCAGGGAGCAAACAGGAGCTAAGTCCAT |
| 1357 | 1361_Bushbaby_monoMut_C36A_n1 | GGGGGAAGCTACTGGTGAATATTAACCAAGGTCACACAGTTATCAGGGAGCAAACAGGAGCTAAGTCCAT |
| 1358 | 1362_Bushbaby_monoMut_C36G_n1 | GGGGGAAGCTACTGGTGAATATTAACCAAGGTCACGCAGTTATCAGGGAGCAAACAGGAGCTAAGTCCAT |
| 1359 | 1363_Bushbaby_monoMut_C36T_n1 | GGGGGAAGCTACTGGTGAATATTAACCAAGGTCACTCAGTTATCAGGGAGCAAACAGGAGCTAAGTCCAT |
| 1360 | 1365_Bushbaby_monoMut_C37G_n1 | GGGGGAAGCTACTGGTGAATATTAACCAAGGTCACCGAGTTATCAGGGAGCAAACAGGAGCTAAGTCCAT |
| 1361 | 1367_Bushbaby_monoMut_A38C_n1 | GGGGGAAGCTACTGGTGAATATTAACCAAGGTCACCCCGTTATCAGGGAGCAAACAGGAGCTAAGTCCAT |
| 1362 | 1368_Bushbaby_monoMut_A38G_n1 | GGGGGAAGCTACTGGTGAATATTAACCAAGGTCACCCGGTTATCAGGGAGCAAACAGGAGCTAAGTCCAT |
| 1363 | 1372_Bushbaby_monoMut_G39T_n1 | GGGGGAAGCTACTGGTGAATATTAACCAAGGTCACCCATTTATCAGGGAGCAAACAGGAGCTAAGTCCAT |
| 1364 | 1375_Bushbaby_monoMut_T40G_n1 | GGGGGAAGCTACTGGTGAATATTAACCAAGGTCACCCAGGTATCAGGGAGCAAACAGGAGCTAAGTCCAT |
| 1365 | 1380_Bushbaby_monoMut_A42G_n1 | GGGGGAAGCTACTGGTGAATATTAACCAAGGTCACCCAGTIGTCAGGGAGCAAACAGGAGCTAAGTCCAT |
| 1366 | 1383_Bushbaby_monoMut_T43C_n1 | GGGGGAAGCTACTGGTGAATATTAACCAAGGTCACCCAGTTACCAGGGAGCAAACAGGAGCTAAGTCCAT |
| 1367 | 1384_Bushbaby_monoMut_T43G_n1 | GGGGGAAGCTACTGGTGAATATTAACCAAGGTCACCCAGTTAGCAGGGAGCAAACAGGAGCTAAGTCCAT |
| 1368 | 1390_Bushbaby_monoMut_A45T_n1 | GGGGGAAGCTACTGGTGAATATTAACCAAGGTCACCCAGTTATCTGGGAGCAAACAGGAGCTAAGTCCAT |
| 1369 | 1393_Bushbaby_monoMut_G46T_n1 | GGGGGAAGCTACTGGTGAATATTAACCAAGGTCACCCAGTTATCATGGAGCAAACAGGAGCTAAGTCCAT |
| 1370 | 1394_Bushbaby_monoMut_G47A_n1 | GGGGGAAGCTACTGGTGAATATTAACCAAGGTCACCCAGTTATCAGAGAGCAAACAGGAGCTAAGTCCAT |
| 1371 | 1396_Bushbaby_monoMut_G47T_n1 | GGGGGAAGCTACTGGTGAATATTAACCAAGGTCACCCAGTTATCAGTGAGCAAACAGGAGCTAAGTCCAT |
| 1372 | 1397_Bushbaby_monoMut_G48A_n1 | GGGGGAAGCTACTGGTGAATATTAACCAAGGTCACCCAGTTATCAGGAAGCAAACAGGAGCTAAGTCCAT |
| 1373 | 1402_Bushbaby_monoMut_A49T_n1 | GGGGGAAGCTACTGGTGAATATTAACCAAGGTCACCCAGTTATCAGGGTGCAAACAGGAGCTAAGTCCAT |
| 1374 | 1405_Bushbaby_monoMut_G50T_n1 | GGGGGAAGCTACTGGTGAATATTAACCAAGGICACCCAGTTATCAGGGATCAAACAGGAGCTAAGTCCAT |
| 1375 | 1411_Bushbaby_monoMut_A52T_n1 | GGGGGAAGCTACTGGTGAATATTAACCAAGGTCACCCAGTTATCAGGGAGCTAACAGGAGCTAAGTCCAT |
| 1376 | 1413_Bushbaby_monoMut_A53G_n1 | GGGGGAAGCTACTGGTGAATATTAACCAAGGTCACCCAGTTATCAGGGAGCAGACAGGAGCTAAGTCCAT |
| 1377 | 1424_Bushbaby_monoMut_G57A_n1 | GGGGGAAGCTACTGGTGAATATTAACCAAGGTCACCCAGTTATCAGGGAGCAAACAAGAGCTAAGTCCAT |
| 1378 | 1431_Bushbaby_monoMut_A59G_n1 | GGGGGAAGCTACTGGTGAATATTAACCAAGGTCACCCAGTTATCAGGGAGCAAACAGGGGCTAAGTCCAT |
| 1379 | 1432_Bushbaby_monoMut_A59T_n1 | GGGGGAAGCTACTGGTGAATATTAACCAAGGTCACCCAGTTATCAGGGAGCAAACAGGTGCTAAGTCCAT |
| 1380 | 1433_Bushbaby_monoMut_G60A_n1 | GGGGGAAGCTACTGGTGAATATTAACCAAGGTCACCCAGTTATCAGGGAGCAAACAGGAACTAAGTCCAT |
| 1381 | 1439_Bushbaby_monoMut_T62A_n1 | GGGGGAAGCTACTGGTGAATATTAACCAAGGTCACCCAGTTATCAGGGAGCAAACAGGAGCAAAGTCCAT |
| 1382 | 1441_Bushbaby_monoMut_T62G_n1 | GGGGGAAGCTACTGGTGAATATTAACCAAGGTCACCCAGTTATCAGGGAGCAAACAGGAGCGAAGTCCAT |
| 1383 | 1443_Bushbaby_monoMut_A63G_n1 | GGGGGAAGCTACTGGTGAATATTAACCAAGGTCACCCAGTTATCAGGGAGCAAACAGGAGCTGAGTCCAT |
| 1384 | 1457_Bushbaby_monoMut_C68A_n1 | GGGGGAAGCTACTGGTGAATATTAACCAAGGTCACCCAGTTATCAGGGAGCAAACAGGAGCTAAGTCAAT |
| 1385 | 1463_Bushbaby_monoMut_T70A_n1 | GGGGGAAGCTACTGGTGAATATTAACCAAGGTCACCCAGTTATCAGGGAGCAAACAGGAGCTAAGTCCAA |
| 1386 | 1464_Bushbaby_monoMut_T70C_n1 | GGGGGAAGCTACTGGTGAATATTAACCAAGGTCACCCAGTTATCAGGGAGCAAACAGGAGCTAAGTCCAC |
| 1387 | 1467_Bushbaby_diMut_GG1AC_n1 | ACGGGAAGCTACTGGTGAATATTAACCAAGGTCACCCAGTTATCAGGGAGCAAACAGGAGCTAAGTCCAT |
| 1388 | 1470_Bushbaby_diMut_GG1CC_n1 | CCGGGAAGCTACTGGTGAATATTAACCAAGGTCACCCAGTTATCAGGGAGCAAACAGGAGCTAAGTCCAT |
| 1389 | 1476_Bushbaby_diMut_GG2AC_n1 | GACGGAAGCTACTGGTGAATATTAACCAAGGTCACCCAGTTATCAGGGAGCAAACAGGAGCTAAGTCCAT |
| 1390 | 1478_Bushbaby_diMut_GG2CA_n1 | GCAGGAAGCTACTGGTGAATATTAACCAAGGTCACCCAGTTATCAGGGAGCAAACAGGAGCTAAGTCCAT |
| 1391 | 1479_Bushbaby_diMut_GG2CC_n1 | GCCGGAAGCTACTGGTGAATATTAACCAAGGTCACCCAGTTATCAGGGAGCAAACAGGAGCTAAGTCCAT |
| 1392 | 1482_Bushbaby_diMut_GG2TC_n1 | GTCGGAAGCTACTGGTGAATATTAACCAAGGTCACCCAGTTATCAGGGAGCAAACAGGAGCTAAGTCCAT |
| 1393 | 1484_Bushbaby_diMut_GG3AA_n1 | GGAAGAAGCTACTGGTGAATATTAACCAAGGTCACCCAGTTATCAGGGAGCAAACAGGAGCTAAGTCCAT |
| 1394 | 1495_Bushbaby_diMut_GG4AT_n1 | GGGATAAGCTACTGGTGAATATTAACCAAGGTCACCCAGTTATCAGGGAGCAAACAGGAGCTAAGTCCAT |
| 1395 | 1497_Bushbaby_diMut_GG4CC_n1 | GGGCCAAGCTACTGGTGAATATTAACCAAGGTCACCCAGTTATCAGGGAGCAAACAGGAGCTAAGTCCAT |
| 1396 | 1506_Bushbaby_diMut_GA5CG_n1 | GGGGCGAGCTACTGGTGAATATTAACCAAGGTCACCCAGTTATCAGGGAGCAAACAGGAGCTAAGTCCAT |
| 1397 | 1507_Bushbaby_diMut_GA5CT_n1 | GGGGCTAGCTACTGGTGAATATTAACCAAGGTCACCCAGTTATCAGGGAGCAAACAGGAGCTAAGTCCAT |
| 1398 | 1515_Bushbaby_diMut_AA6GG_n1 | GGGGGGGGCTACTGGTGAATATTAACCAAGGTCACCCAGTTATCAGGGAGCAAACAGGAGCTAAGTCCAT |
| 1399 | 1516_Bushbaby_diMut_AA6GT_n1 | GGGGGGTGCTACTGGTGAATATTAACCAAGGTCACCCAGTTATCAGGGAGCAAACAGGAGCTAAGTCCAT |
| 1400 | 1519_Bushbaby_diMut_AA6TT_n1 | GGGGGTTGCTACTGGTGAATATTAACCAAGGTCACCCAGTTATCAGGGAGCAAACAGGAGCTAAGTCCAT |
| 1401 | 1521_Bushbaby_diMut_AG7CC_n1 | GGGGGACCCTACTGGTGAATATTAACCAAGGTCACCCAGTTATCAGGGAGCAAACAGGAGCTAAGTCCAT |
| 1402 | 1523_Bushbaby_diMut_AG7GA_n1 | GGGGGAGACTACTGGTGAATATTAACCAAGGTCACCCAGTTATCAGGGAGCAAACAGGAGCTAAGTCCAT |
| 1403 | 1525_Bushbaby_diMut_AG7GT_n1 | GGGGGAGTCTACTGGTGAATATTAACCAAGGTCACCCAGTTATCAGGGAGCAAACAGGAGCTAAGTCCAT |
| 1404 | 1529_Bushbaby_diMut_GC8AA_n1 | GGGGGAAAATACTGGTGAATATTAACCAAGGTCACCCAGTTATCAGGGAGCAAACAGGAGCTAAGTCCAT |
| 1405 | 1531_Bushbaby_diMut_GC8AT_n1 | GGGGGAAATTACTGGTGAATATTAACCAAGGTCACCCAGTTATCAGGGAGCAAACAGGAGCTAAGTCCAT |
| 1406 | 1533_Bushbaby_diMut_GC8CG_n1 | GGGGGAACGTACTGGTGAATATTAACCAAGGTCACCCAGTTATCAGGGAGCAAACAGGAGCTAAGTCCAT |
| 1407 | 1535_Bushbaby_diMut_GC8TA_n1 | GGGGGAATATACTGGTGAATATTAACCAAGGTCACCCAGTTATCAGGGAGCAAACAGGAGCTAAGTCCAT |
| 1408 | 1536_Bushbaby_diMut_GC8TG_n1 | GGGGGAATGTACTGGTGAATATTAACCAAGGTCACCCAGTTATCAGGGAGCAAACAGGAGCTAAGTCCAT |
| 1409 | 1537_Bushbaby_diMut_GC8TT_n1 | GGGGGAATTTACTGGTGAATATTAACCAAGGTCACCCAGTTATCAGGGAGCAAACAGGAGCTAAGTCCAT |
| 1410 | 1539_Bushbaby_diMut_CT9AC_n1 | GGGGGAAGACACTGGTGAATATTAACCAAGGTCACCCAGTTATCAGGGAGCAAACAGGAGCTAAGTCCAT |
| 1411 | 1540_Bushbaby_diMut_CT9AG_n1 | GGGGGAAGAGACTGGTGAATATTAACCAAGGTCACCCAGTTATCAGGGAGCAAACAGGAGCTAAGTCCAT |
| 1412 | 1541_Bushbaby_diMut_CT9GA_n1 | GGGGGAAGGAACTGGTGAATATTAACCAAGGTCACCCAGTTATCAGGGAGCAAACAGGAGCTAAGTCCAT |
| 1413 | 1542_Bushbaby_diMut_CT9GC_n1 | GGGGGAAGGCACTGGTGAATATTAACCAAGGTCACCCAGTTATCAGGGAGCAAACAGGAGCTAAGTCCAT |
| 1414 | 1543_Bushbaby_diMut_CT9GG_n1 | GGGGGAAGGGACTGGTGAATATTAACCAAGGTCACCCAGTTATCAGGGAGCAAACAGGAGCTAAGTCCAT |
| 1415 | 1544_Bushbaby_diMut_CT9TA_n1 | GGGGGAAGTAACTGGTGAATATTAACCAAGGTCACCCAGTTATCAGGGAGCAAACAGGAGCTAAGTCCAT |
| 1416 | 1545_Bushbaby_diMut_CT9TC_n1 | GGGGGAAGTCACTGGTGAATATTAACCAAGGTCACCCAGTTATCAGGGAGCAAACAGGAGCTAAGTCCAT |
| 1417 | 1546_Bushbaby_diMut_CT9TG_n1 | GGGGGAAGTGACTGGTGAATATTAACCAAGGTCACCCAGTTATCAGGGAGCAAACAGGAGCTAAGTCCAT |
| 1418 | 1550_Bushbaby_diMut_TA10CC_n1 | GGGGGAAGCCCCTGGTGAATATTAACCAAGGTCACCCAGTTATCAGGGAGCAAACAGGAGCTAAGTCCAT |
| 1419 | 1551_Bushbaby_diMut_TA10CG_n1 | GGGGGAAGCCGCTGGTGAATATTAACCAAGGTCACCCAGTTATCAGGGAGCAAACAGGAGCTAAGTCCAT |
| 1420 | 1553_Bushbaby_diMut_TA10GC_n1 | GGGGGAAGCGCCTGGTGAATATTAACCAAGGTCACCCAGTTATCAGGGAGCAAACAGGAGCTAAGTCCAT |
| 1421 | 1555_Bushbaby_diMut_TA10GT_n1 | GGGGGAAGCGTCTGGTGAATATTAACCAAGGTCACCCAGTTATCAGGGAGCAAACAGGAGCTAAGTCCAT |
| 1422 | 1563_Bushbaby_diMut_AC11TG_n1 | GGGGGAAGCTTGTGGTGAATATTAACCAAGGTCACCCAGTTATCAGGGAGCAAACAGGAGCTAAGTCCAT |
| 1423 | 1564_Bushbaby_diMut_AC11TT_n1 | GGGGGAAGCTTTTGGTGAATATTAACCAAGGTCACCCAGTTATCAGGGAGCAAACAGGAGCTAAGTCCAT |
| 1424 | 1567_Bushbaby_diMut_CT12AG_n1 | GGGGGAAGCTAAGGGTGAATATTAACCAAGGTCACCCAGTTATCAGGGAGCAAACAGGAGCTAAGTCCAT |
| 1425 | 1573_Bushbaby_diMut_CT12TG_n1 | GGGGGAAGCTATGGGTGAATATTAACCAAGGTCACCCAGTTATCAGGGAGCAAACAGGAGCTAAGTCCAT |
| 1426 | 1574_Bushbaby_diMut_TG13AA_n1 | GGGGGAAGCTACAAGTGAATATTAACCAAGGTCACCCAGTTATCAGGGAGCAAACAGGAGCTAAGTCCAT |
| 1427 | 1585_Bushbaby_diMut_GG14AT_n1 | GGGGGAAGCTACTATTGAATATTAACCAAGGTCACCCAGTTATCAGGGAGCAAACAGGAGCTAAGTCCAT |
| 1428 | 1593_Bushbaby_diMut_GT15AC_n1 | GGGGGAAGCTACTGACGAATATTAACCAAGGTCACCCAGTTATCAGGGAGCAAACAGGAGCTAAGTCCAT |
| 1429 | 1598_Bushbaby_diMut_GT15TA_n1 | GGGGGAAGCTACTGTAGAATATTAACCAAGGTCACCCAGTTATCAGGGAGCAAACAGGAGCTAAGTCCAT |
| 1430 | 1600_Bushbaby_diMut_GT15TG_n1 | GGGGGAAGCTACTGTGGAATATTAACCAAGGTCACCCAGTTATCAGGGAGCAAACAGGAGCTAAGTCCAT |
| 1431 | 1601_Bushbaby_diMut_TG16AA_n1 | GGGGGAAGCTACTGGAAAATATTAACCAAGGTCACCCAGTTATCAGGGAGCAAACAGGAGCTAAGTCCAT |
| 1432 | 1617_Bushbaby_diMut_GA17TG_n1 | GGGGGAAGCTACTGGTTGATATTAACCAAGGTCACCCAGTTATCAGGGAGCAAACAGGAGCTAAGTCCAT |
| 1433 | 1622_Bushbaby_diMut_AA18GC_n1 | GGGGGAAGCTACTGGTGGCTATTAACCAAGGTCACCCAGTTATCAGGGAGCAAACAGGAGCTAAGTCCAT |
| 1434 | 1629_Bushbaby_diMut_AT19CC_n1 | GGGGGAAGCTACTGGTGACCATTAACCAAGGTCACCCAGTTATCAGGGAGCAAACAGGAGCTAAGTCCAT |
| 1435 | 1642_Bushbaby_diMut_TA20CT_n1 | GGGGGAAGCTACTGGTGAACTTTAACCAAGGTCACCCAGTTATCAGGGAGCAAACAGGAGCTAAGTCCAT |
| 1436 | 1645_Bushbaby_diMut_TA20GT_n1 | GGGGGAAGCTACTGGTGAAGTTTAACCAAGGTCACCCAGTTATCAGGGAGCAAACAGGAGCTAAGTCCAT |
| 1437 | 1647_Bushbaby_diMut_AT21CC_n1 | GGGGGAAGCTACTGGTGAATCCTAACCAAGGTCACCCAGTTATCAGGGAGCAAACAGGAGCTAAGTCCAT |
| 1438 | 1648_Bushbaby_diMut_AT21CG_n1 | GGGGGAAGCTACTGGTGAATCGTAACCAAGGTCACCCAGTTATCAGGGAGCAAACAGGAGCTAAGTCCAT |
| 1439 | 1652_Bushbaby_diMut_AT21TA_n1 | GGGGGAAGCTACTGGTGAATTATAACCAAGGTCACCCAGTTATCAGGGAGCAAACAGGAGCTAAGTCCAT |
| 1440 | 1653_Bushbaby_diMut_AT21TC_n1 | GGGGGAAGCTACTGGTGAATTCTAACCAAGGTCACCCAGTTATCAGGGAGCAAACAGGAGCTAAGTCCAT |
| 1441 | 1669_Bushbaby_diMut_TA23CT_n1 | GGGGGAAGCTACTGGTGAATATCTACCAAGGTCACCCAGTTATCAGGGAGCAAACAGGAGCTAAGTCCAT |
| 1442 | 1673_Bushbaby_diMut_AA24CC_n1 | GGGGGAAGCTACTGGTGAATATTCCCCAAGGTCACCCAGTTATCAGGGAGCAAACAGGAGCTAAGTCCAT |
| 1443 | 1681_Bushbaby_diMut_AA24TT_n1 | GGGGGAAGCTACTGGTGAATATTTTCCAAGGTCACCCAGTTATCAGGGAGCAAACAGGAGCTAAGTCCAT |
| 1444 | 1684_Bushbaby_diMut_AC25CT_n1 | GGGGGAAGCTACTGGTGAATATTACTCAAGGTCACCCAGTTATCAGGGAGCAAACAGGAGCTAAGTCCAT |
| 1445 | 1686_Bushbaby_diMut_AC25GG_n1 | GGGGGAAGCTACTGGTGAATATTAGGCAAGGTCACCCAGTTATCAGGGAGCAAACAGGAGCTAAGTCCAT |
| 1446 | 1690_Bushbaby_diMut_AC25TT_n1 | GGGGGAAGCTACTGGTGAATATTATTCAAGGTCACCCAGTTATCAGGGAGCAAACAGGAGCTAAGTCCAT |
| 1447 | 1716_Bushbaby_diMut_AA28TG_n1 | GGGGGAAGCTACTGGTGAATATTAACCTGGGTCACCCAGTTATCAGGGAGCAAACAGGAGCTAAGTCCAT |
| 1448 | 1727_Bushbaby_diMut_GG30AA_n1 | GGGGGAAGCTACTGGTGAATATTAACCAAAATCACCCAGTTATCAGGGAGCAAACAGGAGCTAAGTCCAT |
| 1449 | 1747_Bushbaby_diMut_TC32AT_n1 | GGGGGAAGCTACTGGTGAATATTAACCAAGGATACCCAGTTATCAGGGAGCAAACAGGAGCTAAGTCCAT |
| 1450 | 1758_Bushbaby_diMut_CA33GG_n1 | GGGGGAAGCTACTGGTGAATATTAACCAAGGTGGCCCAGTTATCAGGGAGCAAACAGGAGCTAAGTCCAT |
| 1451 | 1761_Bushbaby_diMut_CA33TG_n1 | GGGGGAAGCTACTGGTGAATATTAACCAAGGTTGCCCAGTTATCAGGGAGCAAACAGGAGCTAAGTCCAT |
| 1452 | 1768_Bushbaby_diMut_AC34GT_n1 | GGGGGAAGCTACTGGTGAATATTAACCAAGGTCGTCCAGTTATCAGGGAGCAAACAGGAGCTAAGTCCAT |
| 1453 | 1772_Bushbaby_diMut_CC35AA_n1 | GGGGGAAGCTACTGGTGAATATTAACCAAGGTCAAACAGTTATCAGGGAGCAAACAGGAGCTAAGTCCAT |
| 1454 | 1780_Bushbaby_diMut_CC35TT_n1 | GGGGGAAGCTACTGGTGAATATTAACCAAGGTCATTCAGTTATCAGGGAGCAAACAGGAGCTAAGTCCAT |
| 1455 | 1785_Bushbaby_diMut_CC36GG_n1 | GGGGGAAGCTACTGGTGAATATTAACCAAGGTCACGGAGTTATCAGGGAGCAAACAGGAGCTAAGTCCAT |
| 1456 | 1786_Bushbaby_diMut_CC36GT_n1 | GGGGGAAGCTACTGGTGAATATTAACCAAGGTCACGTAGTTATCAGGGAGCAAACAGGAGCTAAGTCCAT |
| 1457 | 1787_Bushbaby_diMut_CC36TA_n1 | GGGGGAAGCTACTGGTGAATATTAACCAAGGTCACTAAGTTATCAGGGAGCAAACAGGAGCTAAGTCCAT |
| 1458 | 1792_Bushbaby_diMut_CA37AT_n1 | GGGGGAAGCTACTGGTGAATATTAACCAAGGTCACCATGTTATCAGGGAGCAAACAGGAGCTAAGTCCAT |
| 1459 | 1798_Bushbaby_diMut_CA37TT_n1 | GGGGGAAGCTACTGGTGAATATTAACCAAGGTCACCTTGTTATCAGGGAGCAAACAGGAGCTAAGTCCAT |
| 1460 | 1800_Bushbaby_diMut_AG38CC_n1 | GGGGGAAGCTACTGGTGAATATTAACCAAGGTCACCCCCTTATCAGGGAGCAAACAGGAGCTAAGTCCAT |
| 1461 | 1802_Bushbaby_diMut_AG38GA_n1 | GGGGGAAGCTACTGGTGAATATTAACCAAGGTCACCCGATTATCAGGGAGCAAACAGGAGCTAAGTCCAT |
| 1462 | 1807_Bushbaby_diMut_AG38TT_n1 | GGGGGAAGCTACTGGTGAATATTAACCAAGGTCACCCTTTTATCAGGGAGCAAACAGGAGCTAAGTCCAT |
| 1463 | 1809_Bushbaby_diMut_GT39AC_n1 | GGGGGAAGCTACTGGTGAATATTAACCAAGGTCACCCAACTATCAGGGAGCAAACAGGAGCTAAGTCCAT |
| 1464 | 1810_Bushbaby_diMut_GT39AG_n1 | GGGGGAAGCTACTGGTGAATATTAACCAAGGTCACCCAAGTATCAGGGAGCAAACAGGAGCTAAGTCCAT |
| 1465 | 1813_Bushbaby_diMut_GT39CG_n1 | GGGGGAAGCTACTGGTGAATATTAACCAAGGTCACCCACGTATCAGGGAGCAAACAGGAGCTAAGTCCAT |
| 1466 | 1816_Bushbaby_diMut_GT39TG_n1 | GGGGGAAGCTACTGGTGAATATTAACCAAGGTCACCCATGTATCAGGGAGCAAACAGGAGCTAAGTCCAT |
| 1467 | 1818_Bushbaby_diMut_TT40AC_n1 | GGGGGAAGCTACTGGTGAATATTAACCAAGGTCACCCAGACATCAGGGAGCAAACAGGAGCTAAGTCCAT |
| 1468 | 1822_Bushbaby_diMut_TT40CG_n1 | GGGGGAAGCTACTGGTGAATATTAACCAAGGTCACCCAGCGATCAGGGAGCAAACAGGAGCTAAGTCCAT |
| 1469 | 1833_Bushbaby_diMut_TA41GG_n1 | GGGGGAAGCTACTGGTGAATATTAACCAAGGTCACCCAGTGGTCAGGGAGCAAACAGGAGCTAAGTCCAT |
| 1470 | 1835_Bushbaby_diMut_AT42CA_n1 | GGGGGAAGCTACTGGTGAATATTAACCAAGGTCACCCAGTTCACAGGGAGCAAACAGGAGCTAAGTCCAT |
| 1471 | 1837_Bushbaby_diMut_AT42CG_n1 | GGGGGAAGCTACTGGTGAATATTAACCAAGGTCACCCAGTTCGCAGGGAGCAAACAGGAGCTAAGTCCAT |
| 1472 | 1839_Bushbaby_diMut_AT42GC_n1 | GGGGGAAGCTACTGGTGAATATTAACCAAGGTCACCCAGTTGCCAGGGAGCAAACAGGAGCTAAGTCCAT |
| 1473 | 1849_Bushbaby_diMut_TC43CT_n1 | GGGGGAAGCTACTGGTGAATATTAACCAAGGTCACCCAGTTACTAGGGAGCAAACAGGAGCTAAGTCCAT |
| 1474 | 1851_Bushbaby_diMut_TC43GG_n1 | GGGGGAAGCTACTGGTGAATATTAACCAAGGTCACCCAGTTAGGAGGGAGCAAACAGGAGCTAAGTCCAT |
| 1475 | 1856_Bushbaby_diMut_CA44GC_n1 | GGGGGAAGCTACTGGTGAATATTAACCAAGGTCACCCAGTTATGCGGGAGCAAACAGGAGCTAAGTCCAT |
| 1476 | 1871_Bushbaby_diMut_GG46AA_n1 | GGGGGAAGCTACTGGTGAATATTAACCAAGGTCACCCAGTTATCAAAGAGCAAACAGGAGCTAAGTCCAT |
| 1477 | 1873_Bushbaby_diMut_GG46AT_n1 | GGGGGAAGCTACTGGTGAATATTAACCAAGGTCACCCAGTTATCAATGAGCAAACAGGAGCTAAGTCCAT |
| 1478 | 1874_Bushbaby_diMut_GG46CA_n1 | GGGGGAAGCTACTGGTGAATATTAACCAAGGTCACCCAGTTATCACAGAGCAAACAGGAGCTAAGTCCAT |
| 1479 | 1875_Bushbaby_diMut_GG46CC_n1 | GGGGGAAGCTACTGGTGAATATTAACCAAGGTCACCCAGTTATCACCGAGCAAACAGGAGCTAAGTCCAT |
| 1480 | 1877_Bushbaby_diMut_GG46TA_n1 | GGGGGAAGCTACTGGTGAATATTAACCAAGGTCACCCAGTTATCATAGAGCAAACAGGAGCTAAGTCCAT |
| 1481 | 1879_Bushbaby_diMut_GG46TT_n1 | GGGGGAAGCTACTGGTGAATATTAACCAAGGTCACCCAGTTATCATTGAGCAAACAGGAGCTAAGTCCAT |
| 1482 | 1880_Bushbaby_diMut_GG47AA_n1 | GGGGGAAGCTACTGGTGAATATTAACCAAGGTCACCCAGTTATCAGAAAGCAAACAGGAGCTAAGTCCAT |
| 1483 | 1881_Bushbaby_diMut_GG47AC_n1 | GGGGGAAGCTACTGGTGAATATTAACCAAGGTCACCCAGTTATCAGACAGCAAACAGGAGCTAAGTCCAT |
| 1484 | 1882_Bushbaby_diMut_GG47AT_n1 | GGGGGAAGCTACTGGTGAATATTAACCAAGGTCACCCAGTTATCAGATAGCAAACAGGAGCTAAGTCCAT |
| 1485 | 1883_Bushbaby_diMut_GG47CA_n1 | GGGGGAAGCTACTGGTGAATATTAACCAAGGTCACCCAGTTATCAGCAAGCAAACAGGAGCTAAGTCCAT |
| 1486 | 1884_Bushbaby_diMut_GG47CC_n1 | GGGGGAAGCTACTGGTGAATATTAACCAAGGTCACCCAGTTATCAGCCAGCAAACAGGAGCTAAGTCCAT |
| 1487 | 1888_Bushbaby_diMut_GG47TT_n1 | GGGGGAAGCTACTGGTGAATATTAACCAAGGTCACCCAGTTATCAGTTAGCAAACAGGAGCTAAGTCCAT |
| 1488 | 1890_Bushbaby_diMut_GA48AG_n1 | GGGGGAAGCTACTGGTGAATATTAACCAAGGTCACCCAGTTATCAGGAGGCAAACAGGAGCTAAGTCCAT |
| 1489 | 1909_Bushbaby_diMut_GC50AT_n1 | GGGGGAAGCTACTGGTGAATATTAACCAAGGTCACCCAGTTATCAGGGAATAAACAGGAGCTAAGTCCAT |
| 1490 | 1918_Bushbaby_diMut_CA51AT_n1 | GGGGGAAGCTACTGGTGAATATTAACCAAGGTCACCCAGTTATCAGGGAGATAACAGGAGCTAAGTCCAT |
| 1491 | 1953_Bushbaby_diMut_CA55AG_n1 | GGGGGAAGCTACTGGTGAATATTAACCAAGGTCACCCAGTTATCAGGGAGCAAAAGGGAGCTAAGTCCAT |
| 1492 | 1959_Bushbaby_diMut_CA55TG_n1 | GGGGGAAGCTACTGGTGAATATTAACCAAGGTCACCCAGTTATCAGGGAGCAAATGGGAGCTAAGTCCAT |
| 1493 | 1988_Bushbaby_diMut_AG59CA_n1 | GGGGGAAGCTACTGGTGAATATTAACCAAGGTCACCCAGTTATCAGGGAGCAAACAGGCACTAAGTCCAT |
| 1494 | 1992_Bushbaby_diMut_AG59GC_n1 | GGGGGAAGCTACTGGTGAATATTAACCAAGGTCACCCAGTTATCAGGGAGCAAACAGGGCCTAAGTCCAT |
| 1495 | 1993_Bushbaby_diMut_AG59GT_n1 | GGGGGAAGCTACTGGTGAATATTAACCAAGGTCACCCAGTTATCAGGGAGCAAACAGGGTCTAAGTCCAT |
| 1496 | 1994_Bushbaby_diMut_AG59TA_n1 | GGGGGAAGCTACTGGTGAATATTAACCAAGGTCACCCAGTTATCAGGGAGCAAACAGGTACTAAGTCCAT |
| 1497 | 1995_Bushbaby_diMut_AG59TC_n1 | GGGGGAAGCTACTGGTGAATATTAACCAAGGTCACCCAGTTATCAGGGAGCAAACAGGTCCTAAGTCCAT |
| 1498 | 2004_Bushbaby_diMut_GC60TG_n1 | GGGGGAAGCTACTGGTGAATATTAACCAAGGTCACCCAGTTATCAGGGAGCAAACAGGATGTAAGTCCAT |
| 1499 | 2012_Bushbaby_diMut_CT61TA_n1 | GGGGGAAGCTACTGGTGAATATTAACCAAGGTCACCCAGTTATCAGGGAGCAAACAGGAGTAAAGTCCAT |
| 1500 | 2016_Bushbaby_diMut_TA62AG_n1 | GGGGGAAGCTACTGGTGAATATTAACCAAGGTCACCCAGTTATCAGGGAGCAAACAGGAGCAGAGTCCAT |
| 1501 | 2017_Bushbaby_diMut_TA62AT_n1 | GGGGGAAGCTACTGGTGAATATTAACCAAGGTCACCCAGTTATCAGGGAGCAAACAGGAGCATAGTCCAT |
| 1502 | 2022_Bushbaby_diMut_TA62GG_n1 | GGGGGAAGCTACTGGTGAATATTAACCAAGGTCACCCAGTTATCAGGGAGCAAACAGGAGCGGAGTCCAT |
| 1503 | 2053_Bushbaby_diMut_TC66AT_n1 | GGGGGAAGCTACTGGTGAATATTAACCAAGGTCACCCAGTTATCAGGGAGCAAACAGGAGCTAAGATCAT |
| 1504 | 2056_Bushbaby_diMut_TC66CT_n1 | GGGGGAAGCTACTGGTGAATATTAACCAAGGTCACCCAGTTATCAGGGAGCAAACAGGAGCTAAGCTCAT |
| 1505 | 2059_Bushbaby_diMut_TC66GT_n1 | GGGGGAAGCTACTGGTGAATATTAACCAAGGTCACCCAGTTATCAGGGAGCAAACAGGAGCTAAGGTCAT |
| 1506 | 2060_Bushbaby_diMut_CC67AA_n1 | GGGGGAAGCTACTGGTGAATATTAACCAAGGTCACCCAGTTATCAGGGAGCAAACAGGAGCTAAGTAAAT |

CTAAG -> CAAAG single nucleotide substitution variant had the highest expression.

TABLE 13
Single and adjacent di-nucleotide substitution variants of human SERPINA1 enhancer with HNF4 and FOXA transcription factor consensus sites
with higher luciferase expression than original sequence SEQ ID NO: 85

| SEQ ID NO: | HNF4_FOXA SERPINA1 enhancer variant | Sequence |
|---|---|---|
| 1507 | 3310_HNF4_FOXA_monoMut_G2C_n1 | GCGGGAGGCTGCTGGTAAACATTAACCAAGGTCACCCCAGTTATCAGAGGAGCAAACAGGGGCAAAGTCCAC |
| 1508 | 3312_HNF4_FOXA_monoMut_G3A_n1 | GGAGGAGGCTGCTGGTAAACATTAACCAAGGTCACCCCAGTTATCAGAGGAGCAAACAGGGGCAAAGTCCAC |
| 1509 | 3315_HNF4_FOXA_monoMut_G4A_n1 | GGGAGAGGCTGCTGGTAAACATTAACCAAGGTCACCCCAGTTATCAGAGGAGCAAACAGGGGCAAAGTCCAC |
| 1510 | 3316_HNF4_FOXA_monoMut_G4C_n1 | GGGCGAGGCTGCTGGTAAACATTAACCAAGGTCACCCCAGTTATCAGAGGAGCAAACAGGGGCAAAGTCCAC |
| 1511 | 3333_HNF4_FOXA_monoMut_T10A_n1 | GGGGGAGGCAGCTGGTAAACATTAACCAAGGTCACCCCAGTTATCAGAGGAGCAAACAGGGGCAAAGTCCAC |
| 1512 | 3334_HNF4_FOXA_monoMut_T10C_n1 | GGGGGAGGCCGCTGGTAAACATTAACCAAGGTCACCCCAGTTATCAGAGGAGCAAACAGGGGCAAAGTCCAC |
| 1513 | 3341_HNF4_FOXA_monoMut_C12T_n1 | GGGGGAGGCTGTTGGTAAACATTAACCAAGGTCACCCCAGTTATCAGAGGAGCAAACAGGGGCAAAGTCCAC |
| 1514 | 3342_HNF4_FOXA_monoMut_T13A_n1 | GGGGGAGGCTGCAGGTAAACATTAACCAAGGTCACCCCAGTTATCAGAGGAGCAAACAGGGGCAAAGTCCAC |
| 1515 | 3343_HNF4_FOXA_monoMut_T13C_n1 | GGGGGAGGCTGCCGGTAAACATTAACCAAGGTCACCCCAGTTATCAGAGGAGCAAACAGGGGCAAAGTCCAC |
| 1516 | 3345_HNF4_FOXA_monoMut_G14A_n1 | GGGGGAGGCTGCTAGTAAACATTAACCAAGGTCACCCCAGTTATCAGAGGAGCAAACAGGGGCAAAGTCCAC |
| 1517 | 3362_HNF4_FOXA_monoMut_A19T_n1 | GGGGGAGGCTGCTGGTAATCATTAACCAAGGTCACCCCAGTTATCAGAGGAGCAAACAGGGGCAAAGTCCAC |
| 1518 | 3369_HNF4_FOXA_monoMut_T22A_n1 | GGGGGAGGCTGCTGGTAAACAATAACCAAGGTCACCCCAGTTATCAGAGGAGCAAACAGGGGCAAAGTCCAC |
| 1519 | 3373_HNF4_FOXA_monoMut_T23C_n1 | GGGGGAGGCTGCTGGTAAACATCAACCAAGGTCACCCCAGTTATCAGAGGAGCAAACAGGGGCAAAGTCCAC |
| 1520 | 3375_HNF4_FOXA_monoMut_A24C_n1 | GGGGGAGGCTGCTGGTAAACATTCACCAAGGTCACCCCAGTTATCAGAGGAGCAAACAGGGGCAAAGTCCAC |
| 1521 | 3376_HNF4_FOXA_monoMut_A24G_n1 | GGGGGAGGCTGCTGGTAAACATTGACCAAGGTCACCCCAGTTATCAGAGGAGCAAACAGGGGCAAAGTCCAC |
| 1522 | 3379_HNF4_FOXA_monoMut_A25G_n1 | GGGGGAGGCTGCTGGTAAACATTAGCCAAGGTCACCCCAGTTATCAGAGGAGCAAACAGGGGCAAAGTCCAC |
| 1523 | 3383_HNF4_FOXA_monoMut_C26T_n1 | GGGGGAGGCTGCTGGTAAACATTAATCAAGGTCACCCCAGTTATCAGAGGAGCAAACAGGGGCAAAGTCCAC |
| 1524 | 3386_HNF4_FOXA_monoMut_C27T_n1 | GGGGGAGGCTGCTGGTAAACATTAACTAAGGTCACCCCAGTTATCAGAGGAGCAAACAGGGGCAAAGTCCAC |
| 1525 | 3408_HNF4_FOXA_monoMut_C35A_n1 | GGGGGAGGCTGCTGGTAAACATTAACCAAGGTCAACCCAGTTATCAGAGGAGCAAACAGGGGCAAAGTCCAC |
| 1526 | 3409_HNF4_FOXA_monoMut_C35G_n1 | GGGGGAGGCTGCTGGTAAACATTAACCAAGGTCAGCCCAGTTATCAGAGGAGCAAACAGGGGCAAAGTCCAC |
| 1527 | 3410_HNF4_FOXA_monoMut_C35T_n1 | GGGGGAGGCTGCTGGTAAACATTAACCAAGGTCATCCCAGTTATCAGAGGAGCAAACAGGGGCAAAGTCCAC |
| 1528 | 3413_HNF4_FOXA_monoMut_C36T_n1 | GGGGGAGGCTGCTGGTAAACATTAACCAAGGTCACTCCAGTTATCAGAGGAGCAAACAGGGGCAAAGTCCAC |
| 1529 | 3415_HNF4_FOXA_monoMut_C37G_n1 | GGGGGAGGCTGCTGGTAAACATTAACCAAGGTCACCGCAGTTATCAGAGGAGCAAACAGGGGCAAAGTCCAC |
| 1530 | 3416_HNF4_FOXA_monoMut_C37T_n1 | GGGGGAGGCTGCTGGTAAACATTAACCAAGGTCACCTCAGTTATCAGAGGAGCAAACAGGGGCAAAGTCCAC |
| 1531 | 3418_HNF4_FOXA_monoMut_C38G_n1 | GGGGGAGGCTGCTGGTAAACATTAACCAAGGTCACCCGAGTTATCAGAGGAGCAAACAGGGGCAAAGTCCAC |
| 1532 | 3420_HNF4_FOXA_monoMut_A39C_n1 | GGGGGAGGCTGCTGGTAAACATTAACCAAGGTCACCCCCGTTATCAGAGGAGCAAACAGGGGCAAAGTCCAC |
| 1533 | 3421_HNF4_FOXA_monoMut_A39G_n1 | GGGGGAGGCTGCTGGTAAACATTAACCAAGGTCACCCCGGTTATCAGAGGAGCAAACAGGGGCAAAGTCCAC |
| 1534 | 3422_HNF4_FOXA_monoMut_A39T_n1 | GGGGGAGGCTGCTGGTAAACATTAACCAAGGTCACCCCTGTTATCAGAGGAGCAAACAGGGGCAAAGTCCAC |
| 1535 | 3423_HNF4_FOXA_monoMut_G40A_n1 | GGGGGAGGCTGCTGGTAAACATTAACCAAGGTCACCCCAATTATCAGAGGAGCAAACAGGGGCAAAGTCCAC |
| 1536 | 3427_HNF4_FOXA_monoMut_T41C_n1 | GGGGGAGGCTGCTGGTAAACATTAACCAAGGTCACCCCAGCTATCAGAGGAGCAAACAGGGGCAAAGTCCAC |
| 1537 | 3440_HNF4_FOXA_monoMut_C45T_n1 | GGGGGAGGCTGCTGGTAAACATTAACCAAGGTCACCCCAGTTATTAGAGGAGCAAACAGGGGCAAAGTCCAC |
| 1538 | 3445_HNF4_FOXA_monoMut_G47C_n1 | GGGGGAGGCTGCTGGTAAACATTAACCAAGGTCACCCCAGTTATCACAGGAGCAAACAGGGGCAAAGTCCAC |
| 1539 | 3446_HNF4_FOXA_monoMut_G47T_n1 | GGGGGAGGCTGCTGGTAAACATTAACCAAGGTCACCCCAGTTATCATAGGAGCAAACAGGGGCAAAGTCCAC |
| 1540 | 3447_HNF4_FOXA_monoMut_A48C_n1 | GGGGGAGGCTGCTGGTAAACATTAACCAAGGTCACCCCAGTTATCAGCGGAGCAAACAGGGGCAAAGTCCAC |
| 1541 | 3453_HNF4_FOXA_monoMut_G50A_n1 | GGGGGAGGCTGCTGGTAAACATTAACCAAGGTCACCCCAGTTATCAGAGAAGCAAACAGGGGCAAAGTCCAC |
| 1542 | 3456_HNF4_FOXA_monoMut_A51C_n1 | GGGGGAGGCTGCTGGTAAACATTAACCAAGGTCACCCCAGTTATCAGAGGCGCAAACAGGGGCAAAGTCCAC |
| 1543 | 3459_HNF4_FOXA_monoMut_G52A_n1 | GGGGGAGGCTGCTGGTAAACATTAACCAAGGTCACCCCAGTTATCAGAGGAACAAACAGGGGCAAAGTCCAC |
| 1544 | 3460_HNF4_FOXA_monoMut_G52C_n1 | GGGGGAGGCTGCTGGTAAACATTAACCAAGGTCACCCCAGTTATCAGAGGACCAAACAGGGGCAAAGTCCAC |
| 1545 | 3461_HNF4_FOXA_monoMut_G52T_n1 | GGGGGAGGCTGCTGGTAAACATTAACCAAGGTCACCCCAGTTATCAGAGGATCAAACAGGGGCAAAGTCCAC |
| 1546 | 3464_HNF4_FOXA_monoMut_C53T_n1 | GGGGGAGGCTGCTGGTAAACATTAACCAAGGTCACCCCAGTTATCAGAGGAGTAAACAGGGGCAAAGTCCAC |
| 1547 | 3467_HNF4_FOXA_monoMut_A54T_n1 | GGGGGAGGCTGCTGGTAAACATTAACCAAGGTCACCCCAGTTATCAGAGGAGCTAACAGGGGCAAAGTCCAC |
| 1548 | 3478_HNF4_FOXA_monoMut_A58G_n1 | GGGGGAGGCTGCTGGTAAACATTAACCAAGGTCACCCCAGTTATCAGAGGAGCAAACGGGGGCAAAGTCCAC |
| 1549 | 3488_HNF4_FOXA_monoMut_G61T_n1 | GGGGGAGGCTGCTGGTAAACATTAACCAAGGTCACCCCAGTTATCAGAGGAGCAAACAGGTGCAAAGTCCAC |
| 1550 | 3512_HNF4_FOXA_monoMut_C69T_n1 | GGGGGAGGCTGCTGGTAAACATTAACCAAGGTCACCCCAGTTATCAGAGGAGCAAACAGGGGCAAAGTTCAC |
| 1551 | 3519_HNF4_FOXA_monoMut_C72A_n1 | GGGGGAGGCTGCTGGTAAACATTAACCAAGGTCACCCCAGTTATCAGAGGAGCAAACAGGGGCAAAGTCCAA |
| 1552 | 3521_HNF4_FOXA_monoMut_C72T_n1 | GGGGGAGGCTGCTGGTAAACATTAACCAAGGTCACCCCAGTTATCAGAGGAGCAAACAGGGGCAAAGTCCAT |
| 1553 | 3523_HNF4_FOXA_diMut_GG1AC_n1 | ACGGGAGGCTGCTGGTAAACATTAACCAAGGTCACCCCAGTTATCAGAGGAGCAAACAGGGGCAAAGTCCAC |
| 1554 | 3526_HNF4_FOXA_diMut_GG1CC_n1 | CCGGGAGGCTGCTGGTAAACATTAACCAAGGTCACCCCAGTTATCAGAGGAGCAAACAGGGGCAAAGTCCAC |
| 1555 | 3532_HNF4_FOXA_diMut_GG2AC_n1 | GACGGAGGCTGCTGGTAAACATTAACCAAGGTCACCCCAGTTATCAGAGGAGCAAACAGGGGCAAAGTCCAC |
| 1556 | 3535_HNF4_FOXA_diMut_GG2CC_n1 | GCCGGAGGCTGCTGGTAAACATTAACCAAGGTCACCCCAGTTATCAGAGGAGCAAACAGGGGCAAAGTCCAC |
| 1557 | 3539_HNF4_FOXA_diMut_GG2TT_n1 | GTTGGAGGCTGCTGGTAAACATTAACCAAGGTCACCCCAGTTATCAGAGGAGCAAACAGGGGCAAAGTCCAC |
| 1558 | 3540_HNF4_FOXA_diMut_GG3AA_n1 | GGAAGAGGCTGCTGGTAAACATTAACCAAGGTCACCCCAGTTATCAGAGGAGCAAACAGGGGCAAAGTCCAC |
| 1559 | 3542_HNF4_FOXA_diMut_GG3AT_n1 | GGATGAGGCTGCTGGTAAACATTAACCAAGGTCACCCCAGTTATCAGAGGAGCAAACAGGGGCAAAGTCCAC |
| 1560 | 3545_HNF4_FOXA_diMut_GG3CT_n1 | GGCTGAGGCTGCTGGTAAACATTAACCAAGGTCACCCCAGTTATCAGAGGAGCAAACAGGGGCAAAGTCCAC |
| 1561 | 3546_HNF4_FOXA_diMut_GG3TA_n1 | GGTAGAGGCTGCTGGTAAACATTAACCAAGGTCACCCCAGTTATCAGAGGAGCAAACAGGGGCAAAGTCCAC |
| 1562 | 3550_HNF4_FOXA_diMut_GG4AC_n1 | GGGACAGGCTGCTGGTAAACATTAACCAAGGTCACCCCAGTTATCAGAGGAGCAAACAGGGGCAAAGTCCAC |
| 1563 | 3552_HNF4_FOXA_diMut_GG4CA_n1 | GGGCAAGGCTGCTGGTAAACATTAACCAAGGTCACCCCAGTTATCAGAGGAGCAAACAGGGGCAAAGTCCAC |
| 1564 | 3560_HNF4_FOXA_diMut_GA5AT_n1 | GGGGATGGCTGCTGGTAAACATTAACCAAGGTCACCCCAGTTATCAGAGGAGCAAACAGGGGCAAAGTCCAC |
| 1565 | 3561_HNF4_FOXA_diMut_GA5CC_n1 | GGGGCCGGCTGCTGGTAAACATTAACCAAGGTCACCCCAGTTATCAGAGGAGCAAACAGGGGCAAAGTCCAC |
| 1566 | 3562_HNF4_FOXA_diMut_GA5CG_n1 | GGGGGGGGCTGCTGGTAAACATTAACCAAGGTCACCCCAGTTATCAGAGGAGCAAACAGGGGCAAAGTCCAC |
| 1567 | 3568_HNF4_FOXA_diMut_AG6CC_n1 | GGGGGCCGCTGCTGGTAAACATTAACCAAGGTCACCCCAGTTATCAGAGGAGCAAACAGGGGCAAAGTCCAC |
| 1568 | 3575_HNF4_FOXA_diMut_AG6TT_n1 | GGGGGTTGCTGCTGGTAAACATTAACCAAGGTCACCCCAGTTATCAGAGGAGCAAACAGGGGCAAAGTCCAC |
| 1569 | 3577_HNF4_FOXA_diMut_GG7AC_n1 | GGGGGAACCTGCTGGTAAACATTAACCAAGGTCACCCCAGTTATCAGAGGAGCAAACAGGGGCAAAGTCCAC |
| 1570 | 3584_HNF4_FOXA_diMut_GG7TT_n1 | GGGGGATTCTGCTGGTAAACATTAACCAAGGTCACCCCAGTTATCAGAGGAGCAAACAGGGGCAAAGTCCAC |
| 1571 | 3585_HNF4_FOXA_diMut_GC8AA_n1 | GGGGGAGAATGCTGGTAAACATTAACCAAGGTCACCCCAGTTATCAGAGGAGCAAACAGGGGCAAAGTCCAC |
| 1572 | 3588_HNF4_FOXA_diMut_GC8CA_n1 | GGGGGAGCATGCTGGTAAACATTAACCAAGGTCACCCCAGTTATCAGAGGAGCAAACAGGGGCAAAGTCCAC |
| 1573 | 3589_HNF4_FOXA_diMut_GC8CG_n1 | GGGGGAGCGTGCTGGTAAACATTAACCAAGGTCACCCCAGTTATCAGAGGAGCAAACAGGGGCAAAGTCCAC |
| 1574 | 3591_HNF4_FOXA_diMut_GC8TA_n1 | GGGGGAGTATGCTGGTAAACATTAACCAAGGTCACCCCAGTTATCAGAGGAGCAAACAGGGGCAAAGTCCAC |
| 1575 | 3595_HNF4_FOXA_diMut_CT9AC_n1 | GGGGGAGGACGCTGGTAAACATTAACCAAGGTCACCCCAGTTATCAGAGGAGCAAACAGGGGCAAAGTCCAC |
| 1576 | 3598_HNF4_FOXA_diMut_CT9GC_n1 | GGGGGAGGGCGCTGGTAAACATTAACCAAGGTCACCCCAGTTATCAGAGGAGCAAACAGGGGCAAAGTCCAC |
| 1577 | 3600_HNF4_FOXA_diMut_CT9TA_n1 | GGGGGAGGTAGCTGGTAAACATTAACCAAGGTCACCCCAGTTATCAGAGGAGCAAACAGGGGCAAAGTCCAC |
| 1578 | 3601_HNF4_FOXA_diMut_CT9TC_n1 | GGGGGAGGTCGCTGGTAAACATTAACCAAGGTCACCCCAGTTATCAGAGGAGCAAACAGGGGCAAAGTCCAC |
| 1579 | 3602_HNF4_FOXA_diMut_CT9TG_n1 | GGGGGAGGTGGCTGGTAAACATTAACCAAGGTCACCCCAGTTATCAGAGGAGCAAACAGGGGCAAAGTCCAC |
| 1580 | 3605_HNF4_FOXA_diMut_TG10AT_n1 | GGGGGAGGCATCTGGTAAACATTAACCAAGGTCACCCCAGTTATCAGAGGAGCAAACAGGGGCAAAGTCCAC |
| 1581 | 3608_HNF4_FOXA_diMut_TG10CT_n1 | GGGGGAGGCCTCTGGTAAACATTAACCAAGGTCACCCCAGTTATCAGAGGAGCAAACAGGGGCAAAGTCCAC |
| 1582 | 3609_HNF4_FOXA_diMut_TG10GA_n1 | GGGGGAGGCGACTGGTAAACATTAACCAAGGTCACCCCAGTTATCAGAGGAGCAAACAGGGGCAAAGTCCAC |
| 1583 | 3613_HNF4_FOXA_diMut_GC11AG_n1 | GGGGGAGGCTAGTGGTAAACATTAACCAAGGTCACCCCAGTTATCAGAGGAGCAAACAGGGGCAAAGTCCAC |
| 1584 | 3615_HNF4_FOXA_diMut_GC11CA_n1 | GGGGGAGGCTCATGGTAAACATTAACCAAGGTCACCCCAGTTATCAGAGGAGCAAACAGGGGCAAAGTCCAC |
| 1585 | 3618_HNF4_FOXA_diMut_GC11TA_n1 | GGGGGAGGCTTATGGTAAACATTAACCAAGGTCACCCCAGTTATCAGAGGAGCAAACAGGGGCAAAGTCCAC |
| 1586 | 3621_HNF4_FOXA_diMut_CT12AA_n1 | GGGGGAGGCTGAAGGTAAACATTAACCAAGGTCACCCCAGTTATCAGAGGAGCAAACAGGGGCAAAGTCCAC |
| 1587 | 3622_HNF4_FOXA_diMut_CT12AC_n1 | GGGGGAGGCTGACGGTAAACATTAACCAAGGTCACCCCAGTTATCAGAGGAGCAAACAGGGGCAAAGTCCAC |
| 1588 | 3624_HNF4_FOXA_diMut_CT12GA_n1 | GGGGGAGGCTGGAGGTAAACATTAACCAAGGTCACCCCAGTTATCAGAGGAGCAAACAGGGGCAAAGTCCAC |
| 1589 | 3625_HNF4_FOXA_diMut_CT12GC_n1 | GGGGGAGGCTGGCGGTAAACATTAACCAAGGTCACCCCAGTTATCAGAGGAGCAAACAGGGGCAAAGTCCAC |
| 1590 | 3626_HNF4_FOXA_diMut_CT12GG_n1 | GGGGGAGGCTGGGGGTAAACATTAACCAAGGTCACCCCAGTTATCAGAGGAGCAAACAGGGGCAAAGTCCAC |
| 1591 | 3627_HNF4_FOXA_diMut_CT12TA_n1 | GGGGGAGGCTGTAGGTAAACATTAACCAAGGTCACCCCAGTTATCAGAGGAGCAAACAGGGGCAAAGTCCAC |
| 1592 | 3630_HNF4_FOXA_diMut_TG13AA_n1 | GGGGGAGGCTGCAAGTAAACATTAACCAAGGTCACCCCAGTTATCAGAGGAGCAAACAGGGGCAAAGTCCAC |
| 1593 | 3631_HNF4_FOXA_diMut_TG13AC_n1 | GGGGGAGGCTGCACGTAAACATTAACCAAGGTCACCCCAGTTATCAGAGGAGCAAACAGGGGCAAAGTCCAC |
| 1594 | 3632_HNF4_FOXA_diMut_TG13AT_n1 | GGGGGAGGCTGCATGTAAACATTAACCAAGGTCACCCCAGTTATCAGAGGAGCAAACAGGGGCAAAGTCCAC |
| 1595 | 3635_HNF4_FOXA_diMut_TG13CT_n1 | GGGGGAGGCTGCCTGTAAACATTAACCAAGGTCACCCCAGTTATCAGAGGAGCAAACAGGGGCAAAGTCCAC |
| 1596 | 3637_HNF4_FOXA_diMut_TG13GC_n1 | GGGGGAGGCTGCGCGTAAACATTAACCAAGGTCACCCCAGTTATCAGAGGAGCAAACAGGGGCAAAGTCCAC |
| 1597 | 3638_HNF4_FOXA_diMut_TG13GT_n1 | GGGGGAGGCTGCGTGTAAACATTAACCAAGGTCACCCCAGTTATCAGAGGAGCAAACAGGGGCAAAGTCCAC |
| 1598 | 3649_HNF4_FOXA_diMut_GT15AC_n1 | GGGGGAGGCTGCTGACAAACATTAACCAAGGTCACCCCAGTTATCAGAGGAGCAAACAGGGGCAAAGTCCAC |
| 1599 | 3711_HNF4_FOXA_diMut_TT22AA_n1 | GGGGGAGGCTGCTGGTAAACAAAAACCAAGGTCACCCCAGTTATCAGAGGAGCAAACAGGGGCAAAGTCCAC |
| 1600 | 3713_HNF4_FOXA_diMut_TT22AG_n1 | GGGGGAGGCTGCTGGTAAACAAGAACCAAGGTCACCCCAGTTATCAGAGGAGCAAACAGGGGCAAAGTCCAC |
| 1601 | 3726_HNF4_FOXA_diMut_TA23GC_n1 | GGGGGAGGCTGCTGGTAAACATGCACCAAGGTCACCCCAGTTATCAGAGGAGCAAACAGGGGCAAAGTCCAC |
| 1602 | 3727_HNF4_FOXA_diMut_TA23GG_n1 | GGGGGAGGCTGCTGGTAAACATGGACCAAGGTCACCCCAGTTATCAGAGGAGCAAACAGGGGCAAAGTCCAC |
| 1603 | 3729_HNF4_FOXA_diMut_AA24CC_n1 | GGGGGAGGCTGCTGGTAAACATTCCCCAAGGTCACCCCAGTTATCAGAGGAGCAAACAGGGGCAAAGTCCAC |
| 1604 | 3730_HNF4_FOXA_diMut_AA24CG_n1 | GGGGGAGGCTGCTGGTAAACATTCGCCAAGGTCACCCCAGTTATCAGAGGAGCAAACAGGGGCAAAGTCCAC |
| 1605 | 3732_HNF4_FOXA_diMut_AA24GC_n1 | GGGGGAGGCTGCTGGTAAACATTGCCCAAGGTCACCCCAGTTATCAGAGGAGCAAACAGGGGCAAAGTCCAC |
| 1606 | 3733_HNF4_FOXA_diMut_AA24GG_n1 | GGGGGAGGCTGCTGGTAAACATTGGCCAAGGTCACCCCAGTTATCAGAGGAGCAAACAGGGGCAAAGTCCAC |
| 1607 | 3734_HNF4_FOXA_diMut_AA24GT_n1 | GGGGGAGGCTGCTGGTAAACATTGTCCAAGGTCACCCCAGTTATCAGAGGAGCAAACAGGGGCAAAGTCCAC |
| 1608 | 3735_HNF4_FOXA_diMut_AA24TC_n1 | GGGGGAGGCTGCTGGTAAACATTTCCCAAGGTCACCCCAGTTATCAGAGGAGCAAACAGGGGCAAAGTCCAC |
| 1609 | 3736_HNF4_FOXA_diMut_AA24TG_n1 | GGGGGAGGCTGCTGGTAAACATTTGCCAAGGTCACCCCAGTTATCAGAGGAGCAAACAGGGGCAAAGTCCAC |
| 1610 | 3738_HNF4_FOXA_diMut_AC25CA_n1 | GGGGGAGGCTGCTGGTAAACATTACACAAGGTCACCCCAGTTATCAGAGGAGCAAACAGGGGCAAAGTCCAC |
| 1611 | 3740_HNF4_FOXA_diMut_AC25CT_n1 | GGGGGAGGCTGCTGGTAAACATTACTCAAGGTCACCCCAGTTATCAGAGGAGCAAACAGGGGCAAAGTCCAC |
| 1612 | 3743_HNF4_FOXA_diMut_AC25GT_n1 | GGGGGAGGCTGCTGGTAAACATTAGTCAAGGTCACCCCAGTTATCAGAGGAGCAAACAGGGGCAAAGTCCAC |
| 1613 | 3763_HNF4_FOXA_diMut_CA27TG_n1 | GGGGGAGGCTGCTGGTAAACATTAACTGAGGTCACCCCAGTTATCAGAGGAGCAAACAGGGGCAAAGTCCAC |
| 1614 | 3814_HNF4_FOXA_diMut_CA33GG_n1 | GGGGGAGGCTGCTGGTAAACATTAACCAAGGTGGCCCCAGTTATCAGAGGAGCAAACAGGGGCAAAGTCCAC |
| 1615 | 3823_HNF4_FOXA_diMut_AC34GG_n1 | GGGGGAGGCTGCTGGTAAACATTAACCAAGGTCGGCCCAGTTATCAGAGGAGCAAACAGGGGCAAAGTCCAC |
| 1616 | 3828_HNF4_FOXA_diMut_CC35AA_n1 | GGGGGAGGCTGCTGGTAAACATTAACCAAGGTCAAACCAGTTATCAGAGGAGCAAACAGGGGCAAAGTCCAC |
| 1617 | 3830_HNF4_FOXA_diMut_CC35AT_n1 | GGGGGAGGCTGCTGGTAAACATTAACCAAGGTCAATCCAGTTATCAGAGGAGCAAACAGGGGCAAAGTCCAC |
| 1618 | 3831_HNF4_FOXA_diMut_CC35GA_n1 | GGGGGAGGCTGCTGGTAAACATTAACCAAGGTCAGACCAGTTATCAGAGGAGCAAACAGGGGCAAAGTCCAC |
| 1619 | 3833_HNF4_FOXA_diMut_CC35GT_n1 | GGGGGAGGCTGCTGGTAAACATTAACCAAGGTCAGTCCAGTTATCAGAGGAGCAAACAGGGGCAAAGTCCAC |
| 1620 | 3834_HNF4_FOXA_diMut_CC35TA_n1 | GGGGGAGGCTGCTGGTAAACATTAACCAAGGTCATACCAGTTATCAGAGGAGCAAACAGGGGCAAAGTCCAC |
| 1621 | 3836_HNF4_FOXA_diMut_CC35TT_n1 | GGGGGAGGCTGCTGGTAAACATTAACCAAGGTCATTCCAGTTATCAGAGGAGCAAACAGGGGCAAAGTCCAC |
| 1622 | 3838_HNF4_FOXA_diMut_CC36AG_n1 | GGGGGAGGCTGCTGGTAAACATTAACCAAGGTCACAGCAGTTATCAGAGGAGCAAACAGGGGCAAAGTCCAC |
| 1623 | 3842_HNF4_FOXA_diMut_CC36GT_n1 | GGGGGAGGCTGCTGGTAAACATTAACCAAGGTCACGTCAGTTATCAGAGGAGCAAACAGGGGCAAAGTCCAC |
| 1624 | 3845_HNF4_FOXA_diMut_CC36TT_n1 | GGGGGAGGCTGCTGGTAAACATTAACCAAGGTCACTTCAGTTATCAGAGGAGCAAACAGGGGCAAAGTCCAC |
| 1625 | 3846_HNF4_FOXA_diMut_CC37AA_n1 | GGGGGAGGCTGCTGGTAAACATTAACCAAGGTCACCAAAGTTATCAGAGGAGCAAACAGGGGCAAAGTCCAC |
| 1626 | 3847_HNF4_FOXA_diMut_CC37AG_n1 | GGGGGAGGCTGCTGGTAAACATTAACCAAGGTCACCAGAGTTATCAGAGGAGCAAACAGGGGCAAAGTCCAC |
| 1627 | 3849_HNF4_FOXA_diMut_CC37GA_n1 | GGGGGAGGCTGCTGGTAAACATTAACCAAGGTCACCGAAGTTATCAGAGGAGCAAACAGGGGCAAAGTCCAC |
| 1628 | 3850_HNF4_FOXA_diMut_CC37GG_n1 | GGGGGAGGCTGCTGGTAAACATTAACCAAGGTCACCGGAGTTATCAGAGGAGCAAACAGGGGCAAAGTCCAC |
| 1629 | 3852_HNF4_FOXA_diMut_CC37TA_n1 | GGGGGAGGCTGCTGGTAAACATTAACCAAGGTCACCTAAGTTATCAGAGGAGCAAACAGGGGCAAAGTCCAC |
| 1630 | 3853_HNF4_FOXA_diMut_CC37TG_n1 | GGGGGAGGCTGCTGGTAAACATTAACCAAGGTCACCTGAGTTATCAGAGGAGCAAACAGGGGCAAAGTCCAC |
| 1631 | 3855_HNF4_FOXA_diMut_CA38AC_n1 | GGGGGAGGCTGCTGGTAAACATTAACCAAGGTCACCCACGTTATCAGAGGAGCAAACAGGGGCAAAGTCCAC |
| 1632 | 3856_HNF4_FOXA_diMut_CA38AG_n1 | GGGGGAGGCTGCTGGTAAACATTAACCAAGGTCACCCAGGTTATCAGAGGAGCAAACAGGGGCAAAGTCCAC |
| 1633 | 3857_HNF4_FOXA_diMut_CA38AT_n1 | GGGGGAGGCTGCTGGTAAACATTAACCAAGGTCACCCATGTTATCAGAGGAGCAAACAGGGGCAAAGTCCAC |
| 1634 | 3858_HNF4_FOXA_diMut_CA38GC_n1 | GGGGGAGGCTGCTGGTAAACATTAACCAAGGTCACCCGCGTTATCAGAGGAGCAAACAGGGGCAAAGTCCAC |
| 1635 | 3860_HNF4_FOXA_diMut_CA38GT_n1 | GGGGGAGGCTGCTGGTAAACATTAACCAAGGTCACCCGTGTTATCAGAGGAGCAAACAGGGGCAAAGTCCAC |
| 1636 | 3864_HNF4_FOXA_diMut_AG39CA_n1 | GGGGGAGGCTGCTGGTAAACATTAACCAAGGTCACCCCCATTATCAGAGGAGCAAACAGGGGCAAAGTCCAC |
| 1637 | 3868_HNF4_FOXA_diMut_AG39GC_n1 | GGGGGAGGCTGCTGGTAAACATTAACCAAGGTCACCCCGCTTATCAGAGGAGCAAACAGGGGCAAAGTCCAC |
| 1638 | 3870_HNF4_FOXA_diMut_AG39TA_n1 | GGGGGAGGCTGCTGGTAAACATTAACCAAGGTCACCCCTATTATCAGAGGAGCAAACAGGGGCAAAGTCCAC |
| 1639 | 3871_HNF4_FOXA_diMut_AG39TC_n1 | GGGGGAGGCTGCTGGTAAACATTAACCAAGGTCACCCCTCTTATCAGAGGAGCAAACAGGGGCAAAGTCCAC |
| 1640 | 3872_HNF4_FOXA_diMut_AG39TT_n1 | GGGGGAGGCTGCTGGTAAACATTAACCAAGGTCACCCCTTTTATCAGAGGAGCAAACAGGGGCAAAGTCCAC |
| 1641 | 3880_HNF4_FOXA_diMut_GT40TC_n1 | GGGGGAGGCTGCTGGTAAACATTAACCAAGGTCACCCCATCTATCAGAGGAGCAAACAGGGGCAAAGTCCAC |
| 1642 | 3881_HNF4_FOXA_diMut_GT40TG_n1 | GGGGGAGGCTGCTGGTAAACATTAACCAAGGTCACCCCATGTATCAGAGGAGCAAACAGGGGCAAAGTCCAC |
| 1643 | 3883_HNF4_FOXA_diMut_TT41AC_n1 | GGGGGAGGCTGCTGGTAAACATTAACCAAGGTCACCCCAGACATCAGAGGAGCAAACAGGGGCAAAGTCCAC |
| 1644 | 3884_HNF4_FOXA_diMut_TT41AG_n1 | GGGGGAGGCTGCTGGTAAACATTAACCAAGGTCACCCCAGAGATCAGAGGAGCAAACAGGGGCAAAGTCCAC |
| 1645 | 3887_HNF4_FOXA_diMut_TT41CG_n1 | GGGGGAGGCTGCTGGTAAACATTAACCAAGGTCACCCCAGCGATCAGAGGAGCAAACAGGGGCAAAGTCCAC |
| 1646 | 3889_HNF4_FOXA_diMut_TT41GC_n1 | GGGGGAGGCTGCTGGTAAACATTAACCAAGGTCACCCCAGGCATCAGAGGAGCAAACAGGGGCAAAGTCCAC |
| 1647 | 3890_HNF4_FOXA_diMut_TT41GG_n1 | GGGGGAGGCTGCTGGTAAACATTAACCAAGGTCACCCCAGGGATCAGAGGAGCAAACAGGGGCAAAGTCCAC |
| 1648 | 3897_HNF4_FOXA_diMut_TA42GC_n1 | GGGGGAGGCTGCTGGTAAACATTAACCAAGGTCACCCCAGTGCTCAGAGGAGCAAACAGGGGCAAAGTCCAC |
| 1649 | 3904_HNF4_FOXA_diMut_AT43GC_n1 | GGGGGAGGCTGCTGGTAAACATTAACCAAGGTCACCCCAGTTGCCAGAGGAGCAAACAGGGGCAAAGTCCAC |
| 1650 | 3910_HNF4_FOXA_diMut_TC44AG_n1 | GGGGGAGGCTGCTGGTAAACATTAACCAAGGTCACCCCAGTTAAGAGAGGAGCAAACAGGGGCAAAGTCCAC |
| 1651 | 3913_HNF4_FOXA_diMut_TC44CG_n1 | GGGGGAGGCTGCTGGTAAACATTAACCAAGGTCACCCCAGTTACGAGAGGAGCAAACAGGGGCAAAGTCCAC |
| 1652 | 3917_HNF4_FOXA_diMut_TC44GT_n1 | GGGGGAGGCTGCTGGTAAACATTAACCAAGGTCACCCCAGTTAGTAGAGGAGCAAACAGGGGCAAAGTCCAC |
| 1653 | 3934_HNF4_FOXA_diMut_AG46TC_n1 | GGGGGAGGCTGCTGGTAAACATTAACCAAGGTCACCCCAGTTATCTCAGGAGCAAACAGGGGCAAAGTCCAC |
| 1654 | 3936_HNF4_FOXA_diMut_GA47AC_n1 | GGGGGAGGCTGCTGGTAAACATTAACCAAGGTCACCCCAGTTATCAACGGAGCAAACAGGGGCAAAGTCCAC |
| 1655 | 3938_HNF4_FOXA_diMut_GA47AT_n1 | GGGGGAGGCTGCTGGTAAACATTAACCAAGGTCACCCCAGTTATCAATGGAGCAAACAGGGGCAAAGTCCAC |
| 1656 | 3940_HNF4_FOXA_diMut_GA47CG_n1 | GGGGGAGGCTGCTGGTAAACATTAACCAAGGTCACCCCAGTTATCACGGGAGCAAACAGGGGCAAAGTCCAC |
| 1657 | 3941_HNF4_FOXA_diMut_GA47CT_n1 | GGGGGAGGCTGCTGGTAAACATTAACCAAGGTCACCCCAGTTATCACTGGAGCAAACAGGGGCAAAGTCCAC |
| 1658 | 3945_HNF4_FOXA_diMut_AG48CA_n1 | GGGGGAGGCTGCTGGTAAACATTAACCAAGGTCACCCCAGTTATCAGCAGAGCAAACAGGGGCAAAGTCCAC |
| 1659 | 3947_HNF4_FOXA_diMut_AG48CT_n1 | GGGGGAGGCTGCTGGTAAACATTAACCAAGGTCACCCCAGTTATCAGCTGAGCAAACAGGGGCAAAGTCCAC |
| 1660 | 3948_HNF4_FOXA_diMut_AG48GA_n1 | GGGGGAGGCTGCTGGTAAACATTAACCAAGGTCACCCCAGTTATCAGGAGAGCAAACAGGGGCAAAGTCCAC |
| 1661 | 3949_HNF4_FOXA_diMut_AG48GC_n1 | GGGGGAGGCTGCTGGTAAACATTAACCAAGGTCACCCCAGTTATCAGGCGAGCAAACAGGGGCAAAGTCCAC |
| 1662 | 3950_HNF4_FOXA_diMut_AG48GT_n1 | GGGGGAGGCTGCTGGTAAACATTAACCAAGGTCACCCCAGTTATCAGGTGAGCAAACAGGGGCAAAGTCCAC |
| 1663 | 3951_HNF4_FOXA_diMut_AG48TA_n1 | GGGGGAGGCTGCTGGTAAACATTAACCAAGGTCACCCCAGTTATCAGTAGAGCAAACAGGGGCAAAGTCCAC |
| 1664 | 3952_HNF4_FOXA_diMut_AG48TC_n1 | GGGGGAGGCTGCTGGTAAACATTAACCAAGGTCACCCCAGTTATCAGTCGAGCAAACAGGGGCAAAGTCCAC |
| 1665 | 3953_HNF4_FOXA_diMut_AG48TT_n1 | GGGGGAGGCTGCTGGTAAACATTAACCAAGGTCACCCCAGTTATCAGTTGAGCAAACAGGGGCAAAGTCCAC |
| 1666 | 3954_HNF4_FOXA_diMut_GG49AA_n1 | GGGGGAGGCTGCTGGTAAACATTAACCAAGGTCACCCCAGTTATCAGAAAAGCAAACAGGGGCAAAGTCCAC |
| 1667 | 3955_HNF4_FOXA_diMut_GG49AC_n1 | GGGGGAGGCTGCTGGTAAACATTAACCAAGGTCACCCCAGTTATCAGAACAGCAAACAGGGGCAAAGTCCAC |
| 1668 | 3956_HNF4_FOXA_diMut_GG49AT_n1 | GGGGGAGGCTGCTGGTAAACATTAACCAAGGTCACCCCAGTTATCAGAATAGCAAACAGGGGCAAAGTCCAC |
| 1669 | 3957_HNF4_FOXA_diMut_GG49CA_n1 | GGGGGAGGCTGCTGGTAAACATTAACCAAGGTCACCCCAGTTATCAGACAAGCAAACAGGGGCAAAGTCCAC |
| 1670 | 3959_HNF4_FOXA_diMut_GG49CT_n1 | GGGGGAGGCTGCTGGTAAACATTAACCAAGGTCACCCCAGTTATCAGACTAGCAAACAGGGGCAAAGTCCAC |
| 1671 | 3960_HNF4_FOXA_diMut_GG49TA_n1 | GGGGGAGGCTGCTGGTAAACATTAACCAAGGTCACCCCAGTTATCAGATAAGCAAACAGGGGCAAAGTCCAC |
| 1672 | 3967_HNF4_FOXA_diMut_GA50CG_n1 | GGGGGAGGCTGCTGGTAAACATTAACCAAGGTCACCCCAGTTATCAGAGCGGCAAACAGGGGCAAAGTCCAC |
| 1673 | 3968_HNF4_FOXA_diMut_GA50CT_n1 | GGGGGAGGCTGCTGGTAAACATTAACCAAGGTCACCCCAGTTATCAGAGCTGCAAACAGGGGCAAAGTCCAC |
| 1674 | 3969_HNF4_FOXA_diMut_GA50TC_n1 | GGGGGAGGCTGCTGGTAAACATTAACCAAGGTCACCCCAGTTATCAGAGTCGCAAACAGGGGCAAAGTCCAC |
| 1675 | 3972_HNF4_FOXA_diMut_AG51CA_n1 | GGGGGAGGCTGCTGGTAAACATTAACCAAGGTCACCCCAGTTATCAGAGGCACAAACAGGGGCAAAGTCCAC |
| 1676 | 3973_HNF4_FOXA_diMut_AG51CC_n1 | GGGGGAGGCTGCTGGTAAACATTAACCAAGGTCACCCCAGTTATCAGAGGCCCAAACAGGGGCAAAGTCCAC |
| 1677 | 3975_HNF4_FOXA_diMut_AG51GA_n1 | GGGGGAGGCTGCTGGTAAACATTAACCAAGGTCACCCCAGTTATCAGAGGGACAAACAGGGGCAAAGTCCAC |
| 1678 | 3979_HNF4_FOXA_diMut_AG51TC_n1 | GGGGGAGGCTGCTGGTAAACATTAACCAAGGTCACCCCAGTTATCAGAGGTCCAAACAGGGGCAAAGTCCAC |
| 1679 | 3982_HNF4_FOXA_diMut_GC52AG_n1 | GGGGGAGGCTGCTGGTAAACATTAACCAAGGTCACCCCAGTTATCAGAGGAAGAAACAGGGGCAAAGTCCAC |
| 1680 | 3989_HNF4_FOXA_diMut_GC52TT_n1 | GGGGGAGGCTGCTGGTAAACATTAACCAAGGTCACCCCAGTTATCAGAGGATTAAACAGGGGCAAAGTCCAC |
| 1682 | 4000_HNF4_FOXA_diMut_AA54CG_n1 | GGGGGAGGCTGCTGGTAAACATTAACCAAGGTCACCCCAGTTATCAGAGGAGCCGACAGGGGCAAAGTCCAC |
| 1682 | 4007_HNF4_FOXA_diMut_AA54TT_n1 | GGGGGAGGCTGCTGGTAAACATTAACCAAGGTCACCCCAGTTATCAGAGGAGCTTACAGGGGCAAAGTCCAC |
| 1683 | 4012_HNF4_FOXA_diMut_AA55GG_n1 | GGGGGAGGCTGCTGGTAAACATTAACCAAGGTCACCCCAGTTATCAGAGGAGCAGGCAGGGGCAAAGTCCAC |
| 1684 | 4033_HNF4_FOXA_diMut_CA57TG_n1 | GGGGGAGGCTGCTGGTAAACATTAACCAAGGTCACCCCAGTTATCAGAGGAGCAAATGGGGGCAAAGTCCAC |
| 1685 | 4038_HNF4_FOXA_diMut_AG58GA_n1 | GGGGGAGGCTGCTGGTAAACATTAACCAAGGTCACCCCAGTTATCAGAGGAGCAAACGAGGGCAAAGTCCAC |
| 1686 | 4068_HNF4_FOXA_diMut_GG61TA_n1 | GGGGGAGGCTGCTGGTAAACATTAACCAAGGTCACCCCAGTTATCAGAGGAGCAAACAGGTACAAAGTCCAC |
| 1687 | 4069_HNF4_FOXA_diMut_GG61TC_n1 | GGGGGAGGCTGCTGGTAAACATTAACCAAGGTCACCCCAGTTATCAGAGGAGCAAACAGGTCCAAAGTCCAC |
| 1688 | 4070_HNF4_FOXA_diMut_GG61TT_n1 | GGGGGAGGCTGCTGGTAAACATTAACCAAGGTCACCCCAGTTATCAGAGGAGCAAACAGGTTCAAAGTCCAC |
| 1689 | 4155_HNF4_FOXA_diMut_AC71GA_n1 | GGGGGAGGCTGCTGGTAAACATTAACCAAGGTCACCCCAGTTATCAGAGGAGCAAACAGGGGCAAAGTCCGA |

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A liver-specific nucleic acid regulatory element comprising a nucleic acid sequence having at least 93% identity to a sequence selected from the group consisting of: SEQ ID NOs: 1-80, 138, and 139.

2. The liver-specific nucleic acid regulatory element of claim 1, wherein the nucleic acid sequence has at least 94%, 95%, 96%, 97%,98% or 99% identity to a sequence selected from the group consisting of: SEQ ID NOs: 1-80, 138, and 139.

3-7. (canceled)

8. The liver-specific nucleic acid regulatory element of claim 3, wherein the nucleic acid sequence consists of a sequence selected from the group consisting of: SEQ ID NOs: 1-80, 138, and 139.

9.-21. (canceled)

22. A liver-specific nucleic acid regulatory element comprising a nucleic acid sequence selected from the group consisting of the sequences set forth in Table 10, Table 11, Table 12, and Table 13.

23. The liver-specific nucleic acid regulatory element of claim 22, wherein:
the element comprises at least two nucleic acid sequences selected from the group consisting of the sequences set forth in Table 10, Table 11, Table 12, and Table 13,
the element comprises three (3) nucleic acid sequences selected from the group consisting of the sequences set forth in Table 10, Table 11, Table 12, and Table 13, optionally wherein the three sequences are identical;
the element consists essentially of two (2) to ten (10) nucleic acid sequences selected from the group consisting of the sequences set forth in Table 10, Table 11, Table 12, and Table 13:
the element comprises a spacer placed between the nucleic acid sequences selected from the group consisting of the sequences set forth in Table 10, Table 11, Table 12, and Table 13: or the element comprises a nucleic acid sequence at least 95%, 96%, 97%, 98% or 99% identical to a nucleic acid sequence selected from the group consisting of:
(SEQ ID NO: 223)
GGGGGAGGCTGCTGGTGAATATTAACCAAGGTCACCCCAGTTATCGGAGG
AGCAAACAGGGGCAAAGTCCAC,
(SEQ ID NO: 1381)
GGGGGAAGCTACTGGTGAATATTAACCAAGGTCACCCAGTTATCAGGGAG
CAAACAGGAGCAAAGTCCAT,
(SEQ ID NO: 1073)
GGAGGCTGTTGGTGAATATTAACCAAGGTCACCTCCGTTATCGGAGGAGC
AAACAAGGGCTAAGTCCAC,
and
(SEQ ID NO: 1113)
GGAGGCTGTTGGTGAATATTAACCAAGGTCACCTCAGTTATCGGAGGAGC
AAACAAGGGCAAAGTCCAC.

24.-30. (canceled)

31. A liver-specific expression cassette comprising the liver-specific nucleic acid regulatory element of claim 1, and a liver-specific promoter operably linked to a transgene.

32. (canceled)

33. A liver-specific expression cassette comprising at least three repeats of a liver-specific nucleic acid regulatory element and a liver-specific promoter operably linked to a transgene,
wherein the liver-specific nucleic acid regulatory element comprises a nucleic acid sequence having at least 95% identity to any one of SEQ ID NOs: 81-137, and
wherein two or more nucleotides separate each liver-specific nucleic acid regulatory element.

34. The liver-specific expression cassette of claim 33, wherein:
between 2 and 30 nucleotides separate each regulatory element;
5 nucleotides separate each regulatory element;
11 nucleotides separate each regulatory element; or
30 nucleotides separate each regulatory element.

35.-37. (canceled)

38. The liver-specific expression cassette of claim 33, wherein the liver-specific expression cassette comprises two, three, four, or five repeats of the liver-specific nucleic acid regulatory element or wherein the liver-specific expression cassette comprises six, seven, eight, nine or ten repeats of the liver-specific nucleic acid regulatory element.

39.-41. (canceled)

42. The liver-specific expression cassette of claim 31, wherein the liver-specific promoter is selected from the group consisting of: a transthyretin (TTR) promoter, a minimal TTR promotor (TTRm), an AAT promoter, an albumin (ALB) promotor or minimal promoter, an apolipoprotein A1 (APOA1) promoter or minimal promoter, a complement factor B (CFB) promoter, a ketohexokinase (KHK) promoter, a hemopexin (HPX) promoter or minimal promoter, a nicotinamide N-methyltransferase (NNMT) promoter or minimal promoter, a carboxylesterase 1 (CES1) promoter or minimal promoter, a protein C (PROC) promoter or minimal promoter, an apolipoprotein C3 (APOC3) promoter or minimal promoter, a mannan-binding lectin serine protease 2 (MASP2) promoter or minimal promoter, a hepcidin antimicrobial peptide (HAMP) promoter or minimal promoter, and a serpin peptidase inhibitor, clade C (antithrombin), member 1 (SERPINC1) promoter or minimal promoter.

43. The liver-specific expression cassette of claim 42, wherein the promoter comprises a sequence selected from the group consisting of: SEQ ID NOs 210-216 and 217.

44. (canceled)

45. The liver-specific expression cassette of claim 31, wherein the transgene encodes a liver-specific therapeutic protein.

46. The liver-specific expression cassette of claim 45, wherein the liver-specific therapeutic protein is coagulation factor VIII (FVIII).

47. (canceled)

48. (canceled)

49. A vector comprising the liver-specific nucleic acid regulatory element of claim 1.

50. The vector of claim 49, wherein:
the vector is a viral vector or a non-viral vector,
the vector is a plasmid; or
the vector is a closed-ended DNA (ceDNA) vector.

51. (canceled)

52. (canceled)

53. A pharmaceutical composition comprising the vector of claim 49, and a pharmaceutically acceptable excipient.

54. A method of treating a liver-specific disease or disorder comprising transduction or transfection of the vector according to claim 49, into a subject.

55. The method of claim 54, wherein the subject is a human subject suffering from a genetic disorder.

56. (canceled)

57. (canceled)

58. A method of increasing expression capacity of a liver-specific enhancer element comprising the nucleic acid sequence CTAAG, comprising introducing a single nucleotide substitution (T to A) mutation such that the substitution results in the nucleic acid sequence comprising CAAAG.

59. A liver-specific enhancer element comprising a nucleic acid sequence selected from:
CAAAG; CAAAGT; CAAAGTC; GCAAAGT; GCAAAG;
or
GCAAAGTC.

60. The liver-specific expression cassette of claim 31, which comprises at least two liver-specific nucleic acid regulatory elements or which comprises at least three liver-specific nucleic acid regulatory elements.

61. The liver-specific expression cassette of claim 60, wherein two or more nucleotides separate each of the liver-specific nucleic acid regulatory elements.
